(19) United States
(12) Patent Application Publication
(10) Pub. No.: US 2026/0325685 A1
(43) Pub. Date: Oct. 22, 2026

(54) Radiopharmaceutical Compositions Targeting Carbonic Anhydrase IX and Uses Thereof

(71) Applicant: RayzeBio, Inc., San Diego, CA (US)

(72) Inventors: Andrew R. Hudson, San Diego, CA (US); Nicholas D. Smith, San Diego, CA (US); Joe Anh Tran, San Marcos, CA (US); Yalda Bravo, San Diego, CA (US); Andiliy G. Lai, San Diego, CA (US)

(73) Assignee: RayzeBio, Inc., San Diego, CA (US)

(21) Appl. No.: 19/478,383

(22) PCT Filed: May 1, 2024
(86) PCT No.: PCT/US2024/027301

§ 371 Date: Oct. 24, 2025

Related U.S. Application Data
(60) Provisional application No. 63/499,666, filed on May 2, 2023.

(51) Int. Cl. A61K 51/04

(52) U.S. Cl. CPC A61K 51/0497; A61K 51/0453; A61K 51/0455; A61K 51/0459; A61K 51/0463; A61K 2121/00; A61K 2123/00

(57) ABSTRACT

Described herein are conjugates and compositions targeting carbonic anhydrase IX and uses thereof. The subject conjugates and compositions are useful for the treatment of cancer.

### CROSS-REFERENCE

This application claims the benefit of U.S. Provisional Application Ser. No. 63/499,666 filed May 2, 2023 which is hereby incorporated by reference in its entirety.

### BACKGROUND

The expression of distinct proteins on the surface of tumor cells offers the opportunity to diagnose and characterize disease by probing the phenotypic identity and biochemical composition and activity of the tumor. Radioactive molecules that selectively bind to specific tumor cell surface proteins allow the use of noninvasive imaging techniques, such as molecular imaging or nuclear medicine, for detecting the presence and quantity of tumor associated proteins, thereby providing vital information related to the diagnosis and extent of disease, prognosis, and therapeutic management options. In addition, as radiopharmaceuticals can be prepared that are not only capable of imaging disease but also delivering a therapeutic radionuclide to the diseased tissue, therapy, in particular cancer therapy, can be realized. The selective expression of CA-IX on tumors in response to hypoxia makes it an attractive target to exploit for noninvasive imaging as well as targeted radiotherapy.

In order to grow beyond more than a few millimeters in diameter, tumor micrometastasis need to obtain a supply of oxygen to sustain the high metabolic rate characteristic of rapid growth, and do so by inducing the formation of new blood vessels. The distance that tumor cells reside from blood vessels is inversely proportional to the oxygen pressure of the tumor. Even when angiogenesis occurs and a blood supply is established, the less vascular interior region of the growing tumor mass remains hypoxic and eventually undergoes necrosis.

Hypoxia is associated with a poor response to radiation therapy, and leads to tumor resistance. Since oxygen is necessary for the cytotoxic actions of free radicals generated by radiation, higher, often incompatible, levels of radiation are required to promote damage to the tumor. Therefore, there is a need for non-invasive techniques to stratify patients based on cancer hypoxia who are not expected to respond to radiation therapy because of low oxygen, and who may be candidates for alternative hypoxia-activated chemotherapies that are becoming available. As hypoxia constitutes a major difference between the tumor and normal tissues, it can be exploited for the development of tumor specific probes.

Hypoxia is a potent stimulus for the expression of specific genes, several which function to trigger vasculogenesis and therefore supply oxygen to the tumor, increase metabolism to increase the oxygen extraction factor, and promote a favorable environment for tumor growth. The activation of hypoxia inducible genes is in part mediated by a transcription factor, HIF-1α. Under normoxic conditions, HIF-1α is hydroxylated on proline residues that reside in the oxygen induced degradation domain of the protein by proline hydroxylase. Hydroxyproline facilitates binding of Von-Hippel-Lindau Factor (VHL), a tumor suppressor that, when bound, promotes the ubiquitination and degradation of HIF-1α. During hypoxia, proline hydroxylase is inhibited, and VHL no longer binds HIF-1α; the now stabilized HIF-1α translocates to the nucleus and associates with HIF-1α. This heterodimeric transcription factor then binds to HIF-1 responsive DNA sequences in the promoter region of target genes including the carbonic anhydrase isoform CA-IX, as well as VEGF, erythropoietin, and glucose transporters.

Carbonic anhydrases are a family of enzymes comprised of 16 isozymes that catalyze the reaction: CO₂+H₂O↔HCO₃⁻+H⁺, and therefore play an important role in pH regulation. Specific isozymes are found either in the cytosol, anchored to the membrane, within the mitochondria, or secreted from the cell. The well-studied constitutively expressed isozyme, carbonic anhydrase II, is found in the cytosol of most cell types, and is the primary isoform responsible for the regulation of intracellular pH.

CA-IX is a membrane-anchored isoform of the enzyme with its catalytic domain in the extracellular space. It has a limited tissue distribution and is found at low levels primarily in the gastrointestinal tract. The expression of CA-IX is under the control of HIF-1α, and this isozyme is highly expressed in tumors cells exposed to hypoxia both in vitro and in vivo. Increased CA-IX expression has been detected in carcinomas of the cervix, ovary, kidney, esophagus, lung, breast, and brain. CA-IX has been reported to promote extracellular acidification. The low extracellular pH as a result of the activity of CA-IX leads to tumorigenic transformation, chromosomal rearrangements, extracellular matrix breakdown, migration and invasion, induction of growth factors, protease activation, and chemoresistance.

Currently, many small molecule inhibitors for CA-IX show undesired side effects due to inhibition of other CA isozymes present in the target organ. There is a need for a CA-IX selective inhibitor to potentially reduce undesired side effects that may arise from inhibition of other, including non-membrane-associated, CA isozymes.

### SUMMARY

Disclosed herein is a conjugate of Formula (A), or a pharmaceutically acceptable salt or stereoisomer thereof:

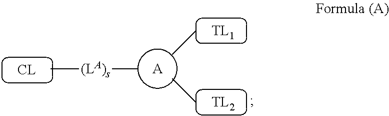

wherein:
   TL₁ represents a 1^{st} targeting moiety that binds to carbonic anhydrase IX;
   TL₂ represents a 2^{nd} targeting moiety that binds to carbonic anhydrase IX;
   L^{A} represents a linker;
   s is 0 or 1;
   Ring A is an optionally substituted cycloalkyl, optionally substituted heterocycloalkyl, optionally substituted aryl, or optionally substituted heteroaryl; and
   CL represents a metal chelator.

Also disclosed herein is a conjugate of Formula (A′), or a pharmaceutically acceptable salt or stereoisomer thereof:

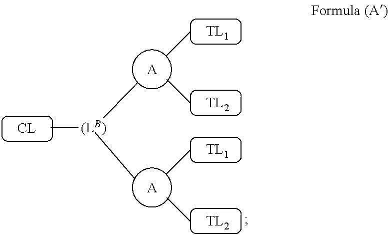

wherein:
   each TL₁ independently represents a 1^{st} targeting moiety that binds to carbonic anhydrase IX;
   each TL₂ independently represents a 2^{nd} targeting moiety that binds to carbonic anhydrase IX;
   L^{B} represents a linker; and
   each Ring A is independently an optionally substituted cycloalkyl, optionally substituted heterocycloalkyl, optionally substituted aryl, or optionally substituted heteroaryl; and
   CL represents a metal chelator.

Also disclosed herein is a conjugate of Formula (B), or a pharmaceutically acceptable salt or stereoisomer thereof:

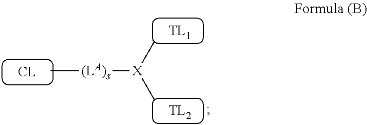

wherein:
   L^{A} represents a linker;
   s is 0 or 1;
   CL represents a metal chelator;
X is N, optionally substituted CH, optionally substituted cycloalkyl, optionally substituted heterocycloalkyl, optionally substituted aryl, or optionally substituted heteroaryl;
TL₁ is of Formula (IB):

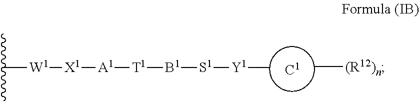

and
TL₂ is of Formula (IIB):

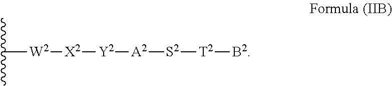

Also provided herein is a conjugate of Formula (C), or a pharmaceutically acceptable salt or stereoisomer thereof:

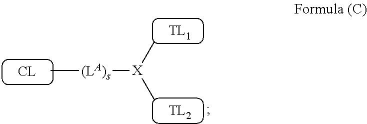

wherein:
   L^{A} represents a linker;
   s is 0 or 1;
   CL represents a metal chelator;
X is N, optionally substituted CH, optionally substituted cycloalkyl, optionally substituted heterocycloalkyl, optionally substituted aryl, or optionally substituted heteroaryl;
TL₁ is of Formula (IC):

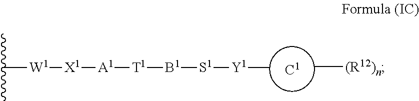

and
TL₂ is of Formula (IIC):

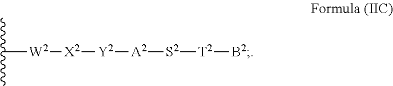

Also disclosed herein is a pharmaceutical composition comprising a radiopharmaceutical conjugate disclosed herein, and a pharmaceutically acceptable excipient or carrier.

In some embodiments, the pharmaceutical composition is formulated for intravenous administration to a subject.

Also disclosed herein is a method of diagnosing or imaging a disease or disorder in a subject in need thereof, the method comprising administering to the subject a radiopharmaceutical conjugate disclosed herein, or a pharmaceutical composition disclosed herein.

Also disclosed herein is a method of treating a disease or disorder in a subject in need thereof, the method comprising administering to the subject a radiopharmaceutical conjugate disclosed herein, or a pharmaceutical composition disclosed herein.

In some embodiments, the disease or disorder is a cancer.

In some embodiments, the cancer is a solid tumor cancer.

In some embodiments, the cancer is hypoxic.

In some embodiments, the cancer is clear cell renal cell carcinoma (ccRCC), endometrial cancer, basal cell carcinoma, liver cancer, colon cancer, gastric cancer, cervical squamous carcinoma, oral squamous cell carcinoma, pancreatic cancer, breast cancer, ovarian cancer, glioblastoma, head and neck cancer, or lung cancer.

In some embodiments, the cancer is cell renal cell carcinoma (ccRCC).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawing (also “figure” and “FIG.” herein), of which:

FIG. 1 to FIG. 19 illustrate the structures of representative metal chelators.

### DETAILED DESCRIPTION

### Definitions

In the following description, certain specific details are set forth to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “some embodiments” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:

“oxo” refers to ═O.

“Carboxyl” refers to —COOH.

“Cyano” refers to —CN.

“Alkyl” refers to a straight-chain, or branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁₋₆alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁₋₁₀alkyl. In some embodiments, the alkyl is a C₁₋₆alkyl. In some embodiments, the alkyl is a C₁₋₅alkyl. In some embodiments, the alkyl is a C₁₋₄alkyl. In some embodiments, the alkyl is a C₁₋₃alkyl. Unless stated otherwise specifically in the specification, an alkyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkyl is optionally substituted with oxo, halogen, —CN, —COOH, —COOMe, —OH, —OMe, —NH₂, or —NO₂. In some embodiments, the alkyl is optionally substituted with halogen, —CN, —OH, or —OMe. In some embodiments, the alkyl is optionally substituted with halogen.

“Alkenyl” refers to a straight-chain, or branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” or “C₂₋₆alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. Unless stated otherwise specifically in the specification, an alkenyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkenyl is optionally substituted with oxo, halogen, —CN, —COOH, —COOMe, —OH, —OMe, —NH₂, or —NO₂. In some embodiments, the alkenyl is optionally substituted with halogen, —CN, —OH, or —OMe. In some embodiments, the alkenyl is optionally substituted with halogen.

“Alkynyl” refers to a straight-chain or branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” or “C₂₋₆alkynyl”, means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. Unless stated otherwise specifically in the specification, an alkynyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkynyl is optionally substituted with oxo, halogen, —CN, —COOH, COOMe, —OH, —OMe, —NH₂, or —NO₂. In some embodiments, the alkynyl is optionally substituted with halogen, —CN, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkylene is optionally substituted with oxo, halogen, —CN, —COOH, COOMe, —OH, —OMe, —NH₂, or —NO₂. In some embodiments, the alkylene is optionally substituted with halogen, —CN, —OH, or —OMe. In some embodiments, the alkylene is optionally substituted with halogen.

“Alkoxy” refers to a radical of the formula-Oalkyl where alkyl is as defined above. Unless stated otherwise specifically in the specification, an alkoxy group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkoxy is optionally substituted with halogen, —CN, —COOH, COOMe, —OH, —OMe, —NH₂, or —NO₂. In some embodiments, the alkoxy is optionally substituted with halogen, —CN, —OH, or —OMe. In some embodiments, the alkoxy is optionally substituted with halogen.

“Aryl” refers to a radical derived from a hydrocarbon ring system comprising 6 to 30 carbon atoms and at least one aromatic ring. The aryl radical may be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl (phenyl). Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the aryl is optionally substituted with halogen, methyl, ethyl, —CN, —COOH, COOMe, —CF₃, —OH, —OMe, —NH₂, or —NO₂. In some embodiments, the aryl is optionally substituted with halogen, methyl, ethyl, —CN, —CF₃, —OH, or —OMe. In some embodiments, the aryl is optionally substituted with halogen.

“Cycloalkyl” refers to a partially or fully saturated, monocyclic, or polycyclic carbocyclic ring, which may include fused (when fused with an aryl or a heteroaryl ring, the cycloalkyl is bonded through a non-aromatic ring atom), spiro, or bridged ring systems. In some embodiments, the cycloalkyl is fully saturated. Representative cycloalkyls include, but are not limited to, cycloalkyls having from three to fifteen carbon atoms (e.g., C₃-C₁₅ fully saturated cycloalkyl or C₃-C₁₅ cycloalkenyl), from three to ten carbon atoms (e.g., C₃-C₁₀ fully saturated cycloalkyl or C₃-C₁₀ cycloalkenyl), from three to eight carbon atoms (e.g., C₃-C₈ fully saturated cycloalkyl or C₃-C₈ cycloalkenyl), from three to six carbon atoms (e.g., C₃-C₆ fully saturated cycloalkyl or C₃-C₆ cycloalkenyl), from three to five carbon atoms (e.g., C₃-C₅ fully saturated cycloalkyl or C₃-C₈ cycloalkenyl), or three to four carbon atoms (e.g., C₃-C₄ fully saturated cycloalkyl or C₃-C₄ cycloalkenyl). In some embodiments, the cycloalkyl is a 3- to 10-membered fully saturated cycloalkyl or a 3- to 10-membered cycloalkenyl. In some embodiments, the cycloalkyl is a 3- to 6-membered fully saturated cycloalkyl or a 3- to 6-membered cycloalkenyl. In some embodiments, the cycloalkyl is a 5- to 6-membered fully saturated cycloalkyl or a 5- to 6-membered cycloalkenyl. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, a cycloalkyl is optionally substituted with oxo, halogen, methyl, ethyl, —CN, —COOH, COOMe, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Halo” or “halogen” refers to bromo, chloro, fluoro or iodo. In some embodiments, halogen is fluoro or chloro. In some embodiments, halogen is fluoro.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, phosphorus, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, phosphorus, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, —CH(CH₃) OCH₃, —CH₂NHCH₃, —CH₂N(CH₃)₂, —CH₂CH₂NHCH₃, or —CH₂CH₂N(CH₃)₂. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Heterocycloalkyl” refers to a 3- to 24-membered partially or fully saturated ring radical comprising 2 to 23 carbon atoms and from one to 8 heteroatoms selected from the group consisting of nitrogen, oxygen, phosphorous, silicon, and sulfur. In some embodiments, the heterocycloalkyl is fully saturated. In some embodiments, the heterocycloalkyl comprises one to three heteroatoms selected from the group consisting of nitrogen, oxygen, and sulfur. In some embodiments, the heterocycloalkyl comprises one to three heteroatoms selected from the group consisting of nitrogen and oxygen. In some embodiments, the heterocycloalkyl comprises one to three nitrogens. In some embodiments, the heterocycloalkyl comprises one or two nitrogens. In some embodiments, the heterocycloalkyl comprises one nitrogen. In some embodiments, the heterocycloalkyl comprises one nitrogen and one oxygen. Unless stated otherwise specifically in the specification, the heterocycloalkyl radical may be a monocyclic, bicyclic, tricyclic, or tetracyclic ring system, which may include fused (when fused with an aryl or a heteroaryl ring, the heterocycloalkyl is bonded through a non-aromatic ring atom), spiro, or bridged ring systems; and the nitrogen, carbon, or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (e.g., C₂-C₁₅ fully saturated heterocycloalkyl or C₂-C₁₅ heterocycloalkenyl), from two to ten carbon atoms (e.g., C₂-C₁₀ fully saturated heterocycloalkyl or C₂-C₁₀ heterocycloalkenyl), from two to eight carbon atoms (e.g., C₂-C₈ fully saturated heterocycloalkyl or C₂-C₈ heterocycloalkenyl), from two to seven carbon atoms (e.g., C₂-C₇ fully saturated heterocycloalkyl or C₂-C₇ heterocycloalkenyl), from two to six carbon atoms (e.g., C₂-C₆ fully saturated heterocycloalkyl or C₂-C₆ heterocycloalkenyl), from two to five carbon atoms (e.g., C₂-C₅ fully saturated heterocycloalkyl or C₂-C₈ heterocycloalkenyl), or two to four carbon atoms (e.g., C₂-C₄ fully saturated heterocycloalkyl or C₂-C₄ heterocycloalkenyl). Examples of such heterocycloalkyl radicals include, but are not limited to, aziridinyl, azetidinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides, and the oligosaccharides. In some embodiments, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). In some embodiments, the heterocycloalkyl is a 3- to 8-membered heterocycloalkyl. In some embodiments, the heterocycloalkyl is a 3- to 7-membered heterocycloalkyl. In some embodiments, the heterocycloalkyl is a 3- to 6-membered heterocycloalkyl. In some embodiments, the heterocycloalkyl is a 4- to 6-membered heterocycloalkyl. In some embodiments, the heterocycloalkyl is a 5- to 6-membered heterocycloalkyl. In some embodiments, the heterocycloalkyl is a 3- to 8-membered heterocycloalkenyl. In some embodiments, the heterocycloalkyl is a 3- to 7-membered heterocycloalkenyl. In some embodiments, the heterocycloalkyl is a 3- to 6-membered heterocycloalkenyl. In some embodiments, the heterocycloalkyl is a 4- to 6-membered heterocycloalkenyl. In some embodiments, the heterocycloalkyl is a 5- to 6-membered heterocycloalkenyl. Unless stated otherwise specifically in the specification, a heterocycloalkyl may be optionally substituted as described below, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heterocycloalkyl is optionally substituted with oxo, halogen, methyl, ethyl, —CN, —COOH, COOMe, —CF₃, —OH, —OMe, —NH₂, or —NO₂. In some embodiments, the heterocycloalkyl is optionally substituted with halogen, methyl, ethyl, —CN, —CF₃, —OH, or —OMe. In some embodiments, the heterocycloalkyl is optionally substituted with halogen.

“Heteroaryl” refers to a 5- to 14-membered ring system radical comprising one to thirteen carbon atoms, one to six heteroatoms selected from the group consisting of nitrogen, oxygen, phosphorous, and sulfur, and at least one aromatic ring. In some embodiments, the heteroaryl comprises one to three heteroatoms selected from the group consisting of nitrogen, oxygen, and sulfur. In some embodiments, the heteroaryl comprises one to three heteroatoms selected from the group consisting of nitrogen and oxygen. In some embodiments, the heteroaryl comprises one to three nitrogens. In some embodiments, the heteroaryl comprises one or two nitrogens. In some embodiments, the heteroaryl comprises one nitrogen. The heteroaryl radical may be a monocyclic, bicyclic, tricyclic, or tetracyclic ring system, which may include fused (when fused with a cycloalkyl or heterocycloalkyl ring, the heteroaryl is bonded through an aromatic ring atom) or bridged ring systems; and the nitrogen, carbon, or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. In some embodiments, the heteroaryl is a 6-membered heteroaryl. In some embodiments, the heteroaryl is a 5-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heteroaryl is optionally substituted with halogen, methyl, ethyl, —CN, —COOH, COOMe, —CF₃, —OH, —OMe, —NH₂, or —NO₂. In some embodiments, the heteroaryl is optionally substituted with halogen, methyl, ethyl, —CN, —CF₃, —OH, or —OMe. In some embodiments, the heteroaryl is optionally substituted with halogen.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.). It will be understood by those skilled in the art with respect to any group containing one or more substituents that such groups are not intended to introduce any substitution or substitution patterns that are sterically impractical and/or synthetically non-feasible. Thus, any substituents described should generally be understood as having a maximum molecular weight of about 1,000 daltons, and more typically, up to about 500 daltons.

The term “one or more” when referring to an optional substituent means that the subject group is optionally substituted with one, two, three, four, or more substituents. In some embodiments, the subject group is optionally substituted with one, two, three or four substituents. In some embodiments, the subject group is optionally substituted with one, two, or three substituents. In some embodiments, the subject group is optionally substituted with one or two substituents. In some embodiments, the subject group is optionally substituted with one substituent. In some embodiments, the subject group is optionally substituted with two substituents.

An “effective amount” or “therapeutically effective amount” refers to an amount of a compound administered to a mammalian subject, either as a single dose or as part of a series of doses, which is effective to produce a desired therapeutic effect.

“Treatment” of an individual (e.g. a mammal, such as a human) or a cell is any type of intervention used in an attempt to alter the natural course of the individual or cell. In some embodiments, treatment includes administration of a pharmaceutical composition, subsequent to the initiation of a pathologic event or contact with an etiologic agent and includes stabilization of the condition (e.g., condition does not worsen) or alleviation of the condition.

### Compounds

Disclosed herein are conjugates targeting the CA-IX receptor and pharmaceutical compositions thereof. In some embodiments, the conjugate disclosed herein is selective for CA-IX over CA-XII.

The conjugates and compositions can be useful for treating cancer. The conjugates and compositions can also be useful in imaging and disease diagnosis.

In one aspect, described herein are conjugates that comprise a targeting ligand that binds to a CA-IX protein. In some embodiments, the conjugates described herein comprise a targeting ligand that binds to a CA-IX protein and a metal chelator that is configured to bind with a radionuclide. In some embodiments, the conjugates described herein comprises a radionuclide.

Disclosed herein is a conjugate of Formula (A), or a pharmaceutically acceptable salt or stereoisomer thereof:

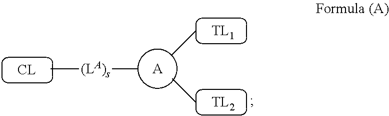

wherein:
   TL₁ represents a 1^{st} targeting moiety that binds to carbonic anhydrase IX;
   TL₂ represents a 2^{nd} targeting moiety that binds to carbonic anhydrase IX;
   L^{A} represents a linker;
   s is 0 or 1;
   Ring A is an optionally substituted cycloalkyl, optionally substituted heterocycloalkyl, optionally substituted aryl, or optionally substituted heteroaryl; and
   CL represents a metal chelator.

Disclosed herein is a conjugate of Formula (AC), or a pharmaceutically acceptable salt or stereoisomer thereof:

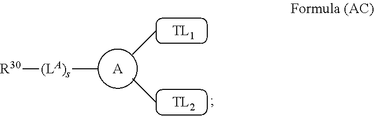

wherein:
   TL₁ represents a 1^{st} targeting moiety that binds to carbonic anhydrase IX;
   TL₂ represents a 2^{nd} targeting moiety that binds to carbonic anhydrase IX;
   L^{A} represents a linker;
   s is 0 or 1;
   Ring A is an optionally substituted cycloalkyl, optionally substituted heterocycloalkyl, optionally substituted aryl, or optionally substituted heteroaryl; and
   R³⁰ is hydrogen, halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NRbC(═O)NR^{c}R^{d}, —NRbC(═O)R^{a}, —NRbC(═O)OR^{b}, —NR^{b}S(═O)₂R^{a}, —C(═O)R^{a}, —C(═O)OR^{b}, —C(═O)NR^{c}R^{d}, C₂-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted (e.g., with one or more R); or
   R³⁰ is an albumin binding moiety or a payload moiety;
   wherein R^{a}, R^{b}, R^{c}, R^{d}, and R have their meanings as defined in Formula (IA).

In some embodiments, the conjugate compound of Formula (AC) comprises a covalently bound radioisotope.

Disclosed herein is a conjugate of Formula (A′), or a pharmaceutically acceptable salt or stereoisomer thereof:

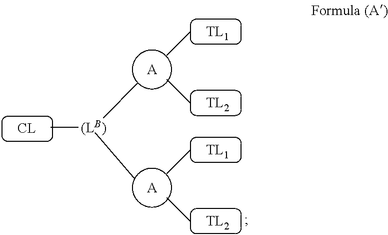

wherein:
   each TL₁ independently represents a 1^{st} targeting moiety that binds to carbonic anhydrase IX;
   each TL₂ independently represents a 2^{nd} targeting moiety that binds to carbonic anhydrase IX;
   L^{B} represents a linker; and
   each Ring A is independently an optionally substituted cycloalkyl, optionally substituted heterocycloalkyl, optionally substituted aryl, or optionally substituted heteroaryl; and
   CL represents a metal chelator.

Disclosed herein is a conjugate of Formula (AC′), or a pharmaceutically acceptable salt or stereoisomer thereof:

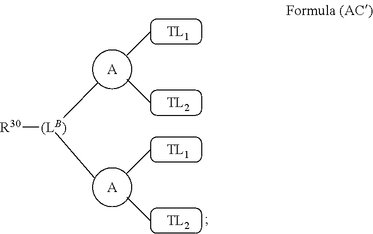

wherein:
   each TL₁ independently represents a 1^{st} targeting moiety that binds to carbonic anhydrase IX;
   each TL₂ independently represents a 2^{nd} targeting moiety that binds to carbonic anhydrase IX;
   L^{B} represents a linker;
   each Ring A is independently an optionally substituted cycloalkyl, optionally substituted heterocycloalkyl, optionally substituted aryl, or optionally substituted heteroaryl; and
   R³⁰ is hydrogen, halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NRbC(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted (e.g., with one or more R); or
   R³⁰ is an albumin binding moiety or a payload moiety;
   wherein R^{a}, R^{b}, R^{c}, R^{d}, and R have their meanings as defined in Formula (IA).

In some embodiments, the conjugate compound of Formula (AC′) comprises a covalently bound radioisotope.

In some embodiments of a compound of Formula (A′) or (AC′), each TL is the same. In some embodiments of a compound of Formula (A′) or (AC′), each TL₁ is different. In some embodiments of a compound of Formula (A′) or (AC′), each TL₂ is the same. In some embodiments of a compound of Formula (A′) or (AC′), each TL₂ is different. In some embodiments of a compound of Formula (A′) or (AC′), each Ring A is the same. In some embodiments of a compound of Formula (A′) or (AC′), each Ring A is different. In some embodiments of a compound of Formula (A′) or (AC′), each TLI is independently a targeting moiety of Formula (IA). In some embodiments of a compound of Formula (A′) or (AC′), each TL₂ is independently a targeting moiety of Formula (IIA).

In some embodiments of a compound of Formula (A), (AC), (A′) or (AC′),
Ring A is cycloalkyl, heterocycloalkyl, aryl, or heteroaryl, wherein the cycloalkyl, heterocycloalkyl, aryl, and heteroaryl is optionally substituted with one or more R; and
each R is independently halogen, —CN, —OH, —SF₅, —SH, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃minoalkyl, C₁-C₃heteroalkyl, C₃-C₆cycloalkyl, or 3- to 6-membered heterocycloalkyl; or two R on the same atom form an oxo.

In some embodiments of a compound of Formula (A), (AC), (A′) or (AC′), Ring A is heterocycloalkyl optionally substituted with one or more R.

In some embodiments of a compound of Formula (A), (AC), (A′) or (AC′), Ring A is monocyclic heterocycloalkyl optionally substituted with one or more R.

In some embodiments of a compound of Formula (A), (AC), (A′) or (AC′), Ring A is bicyclic heterocycloalkyl optionally substituted with one or more R. In some embodiments of a compound of Formula (A), (AC), (A′) or (AC′), Ring A is spirobicyclic heterocycloalkyl optionally substituted with one or more R.

In some embodiments of a compound of Formula (A), (AC), (A′) or (AC′), Ring A is 4- to 6-membered heterocycloalkyl optionally substituted with one or more R. In some embodiments of a compound of Formula (A), (AC), (A′) or (AC′), Ring A is azetidinyl, pyrrolidinyl, piperidinyl, or piperazinyl, each optionally substituted with one or more R. In some embodiments of a compound of Formula (A), (AC), (A′) or (AC′), Ring A is piperidinyl or piperazinyl, each optionally substituted with one or more R. In some embodiments of a compound of Formula (A), (AC), (A′) or (AC′), Ring A is piperidinyl optionally substituted with one or more R. In some embodiments of a compound of Formula (A), (AC), (A′) or (AC′), Ring A is piperazinyl optionally substituted with one or more R.

In some embodiments of a compound of Formula (A), (AC), (A′) or (AC′),

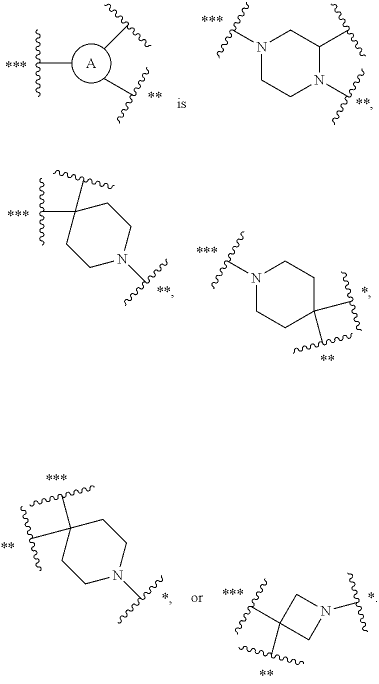

In some embodiments of a compound of Formula (A), (AC), (A′) or (AC′),

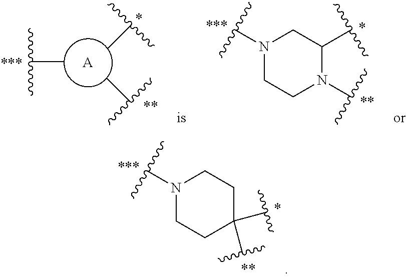

In some embodiments of a compound of Formula (A), (AC), (A′) or (AC′),

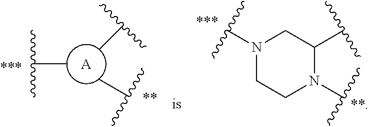

In some embodiments of a compound of Formula (A), (AC), (A′) or (AC′),

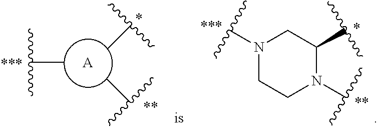

In some embodiments of a compound of Formula (A), (AC), (A′) or (AC′),

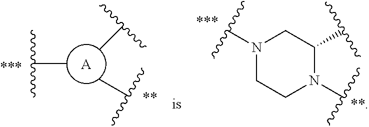

In some embodiments of a compound of Formula (A), (AC), (A′) or (AC′),

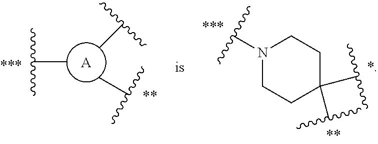

In some embodiments of Formula (A) or Formula (AC), *** represents the attachment point of ring A to linker L^{A}. In some embodiments of Formula (A), when s is 0, *** represents the attachment point of ring A to metal chelator (CL). In some embodiments of Formula (AC), when s is 0, *** represents the attachment point of ring A to R³⁰. In some embodiments of Formula (A) or Formula (AC), ** represents the attachment point of ring A to TL₁, and * represents the attachment point of ring A to TL₂. In some embodiments of Formula (A) or Formula (AC), * represents the attachment point of ring A to TL₁, and ** represents the attachment point of ring A to TL₂. In some embodiments of Formula (A′) or Formula (AC′), *** represents the attachment point of ring A to linker L^{B}. In some embodiments of Formula (A′) or Formula (AC′), ** represents the attachment point of ring A to a TL₁, and * represents the attachment point of ring A to a TL₂. In some embodiments of Formula (A′) or Formula (AC′), * represents the attachment point of ring A to a TL₁, and ** represents the attachment point of ring A to a TL₂.

In some embodiments of a compound of Formula (A), (AC), (A′) or (AC′), TL₁ is of Formula (IA):

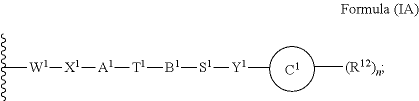

wherein:
W¹ is absent, —O—, —S—, —S(═O)—, —S(═O)₂—, —NR¹¹—, —C(═O)—, —C(═O)NR¹¹—, —NR¹¹C(═O)—, —S(═O)₂NR¹¹—, or —NR¹¹S(═O)₂;
X¹ is C₁-C₁₀alkylene optionally substituted with one or more R and wherein one, two, three, or four carbon atoms of the C₁-C₁₀alkylene are optionally replaced with —S—, —S(═O)—, —S(═O)₂—, —O—, or —NR¹¹—;
A¹ is absent, cycloalkyl, heterocycloalkyl, aryl, or heteroaryl, wherein the cycloalkyl, heterocycloalkyl, aryl, and heteroaryl is optionally substituted with one or more R;
T¹ is absent or C₁-C₃alkylene optionally substituted with one or more R and wherein one carbon atom of the C₁-C₃alkylene is optionally replaced with —C(═O)—, —S—, —S(═O)—, —S(═O)₂—, —O—, or —NR¹¹—;
B¹ is absent, C₁-C₁₀alkylene, cycloalkyl, heterocycloalkyl, aryl, or heteroaryl, wherein the alkylene, cycloalkyl, heterocycloalkyl, aryl, and heteroaryl is optionally substituted with one or more R, and wherein one, two, three, or four carbon atoms in the C₁-C₁₀alkylene are optionally replaced with —S—, —S(═O)—, —S(═O)₂—, —O—, —NR¹¹—;
S¹ is absent or C₁-C₃alkylene optionally substituted with one or more R and wherein one or two carbon atoms of the C₁-C₃alkylene are optionally replaced with —S—, —S(═O)—, —S(═O)₂—, —O—, or —NR¹¹—;
Y¹ is absent, —O—, —S—, —S(═O)—, —S(═O)₂—, —NR¹¹—, —C(═O)—, —C(═O)NR¹¹—, —NR¹¹C(═O)—, —S(═O)₂NR¹¹—, or —NR¹¹S(═O)₂;
R¹¹ is hydrogen, C₁-C₆alkyl, C₁-C₆haloalkyl, C₁-C₆hydroxyalkyl, C₁-C₆aminoalkyl, or C₁-C₆heteroalkyl;
Ring C₁ is cycloalkyl, heterocycloalkyl, aryl, or heteroaryl;
each R¹² is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NRbC(═O)NR^{c}R^{d}, —NRbC(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
n is 0, 1, 2, 3, 4, 5, or 6;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, -L-cycloalkyl, -L-heterocycloalkyl, -L-aryl, or -L-heteroaryl, wherein each alkyl, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, -L-cycloalkyl, -L-heterocycloalkyl, -L-aryl, or -L-heteroaryl, wherein each alkyl, alkenyl, alkynyl, cycloalkyl, heterocycloalkyl, aryl, and heteroaryl is independently optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, -L-cycloalkyl, -L-heterocycloalkyl, -L-aryl, or -L-heteroaryl, wherein each alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
L is absent or C₁-C₃alkylene optionally substituted with one or more R; and
each R is independently halogen, —CN, —OH, —SF₅, —SH, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃minoalkyl, C₁-C₃heteroalkyl, C₃-C₆cycloalkyl, or 3- to 6-membered heterocycloalkyl;
or two R on the same atom form an oxo;
or two R on the same or different atoms are taken together to form a cycloalkyl, heterocycloalkyl, aryl, or heteroaryl.

In some embodiments of a compound of Formula (A), (AC), (A′) or (AC′), TL₁ is of Formula (IA):

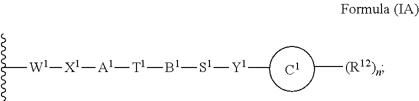

wherein:
W¹ is absent, —C(═O)—, or —C(═O)NR¹¹—;
X¹ is C₁-C₁₀alkylene;
A¹ is absent or heteroaryl optionally substitued with one or more R;
T¹ is absent;
B¹ is C₁-C₁₀alkylene or cycloalkyl;
S¹ is absent;
Y¹ is absent, —O—, —S—, —S(═O)₂—, —C(═O)NR¹¹—, or —NR¹¹C(═O)—;
R¹¹ is hydrogen;
Ring C₁ is heteroaryl;
each R¹² is independently halogen, —S(═O)₂NR^{c}R^{d}, or —NR^{c}R^{d};
n is 0, 1, 2, or 3;
R^{c} and R^{d} are each independently hydrogen, C₁-C₆alkyl, -L-cycloalkyl, or -L-aryl, wherein each alkyl, cycloalkyl, and aryl is independently optionally substituted with one or more R;
L is absent or C₁-C₃alkylene; and
each R is independently C₁-C₃alkyl or C₁-C₃alkoxy.

In some embodiments of a compound of Formula (IA), W¹ is absent, —O—, —S—, —NR¹¹—, —C(═O)—, —C(═O)NR¹¹—, or —NR¹¹C(═O)—.

In some embodiments of a compound of Formula (IA), W¹ is absent, —C(═O)—, or —C(═O)NR¹¹—.

In some embodiments of a compound of Formula (IA), W¹ is absent.

In some embodiments of a compound of Formula (IA), W¹ is —C(═O)—.

In some embodiments of a compound of Formula (IA), W¹ is —C(═O)NR¹¹—.

In some embodiments of a compound of Formula (IA), X¹ is C₁-C₆alkylene optionally substituted with one or more R and wherein one or two carbon atoms of the C₁-C₆alkylene are optionally replaced with —S—, —S(═O)—, —S(═O)₂—, —O—, or —NR¹¹—.

In some embodiments of a compound of Formula (IA), X¹ is C₁-C₆alkylene.

In some embodiments of a compound of Formula (IA), X¹ is C₁-C₄alkylene.

In some embodiments of a compound of Formula (IA), X¹ is —CH₂—.

In some embodiments of a compound of Formula (IA), X¹ is —CH₂CH₂—.

In some embodiments of a compound of Formula (IA), X¹ is —CH₂CH₂CH₂—.

In some embodiments of a compound of Formula (IA), X¹ is —CH₂CH₂CH₂CH₂—.

In some embodiments of a compound of Formula (IA),

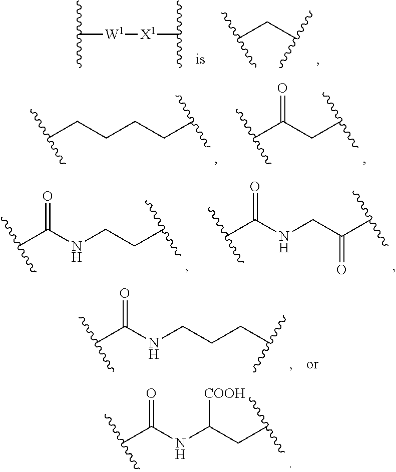

In some embodiments of a compound of Formula (IA),

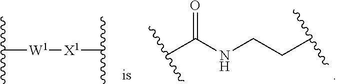

In some embodiments of a compound of Formula (IA), A¹ is absent.

In some embodiments of a compound of Formula (IA), A¹ is heteroaryl optionally substitued with one or more R.

In some embodiments of a compound of Formula (IA), A¹ is triazolyl optionally substitued with one or more R. In some embodiments of a compound of Formula (IA), A¹ is unsubstituted triazolyl.

In some embodiments of a compound of Formula (IA), A¹ is

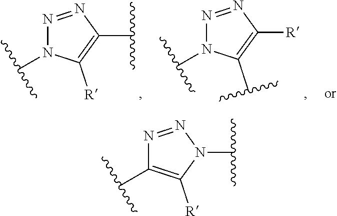

wherein R′ is hydrogen or R. In some embodiments of a compound of Formula (IA), A¹ is

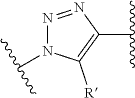

wherein R′ is hydrogen or R. In some embodiments, R′ is hydrogen.

In some embodiments of a compound of Formula (IA), A¹ is heterocycloalkyl optionally substitued with one or more R.

In some embodiments of a compound of Formula (IA), A¹ is monocyclic heterocycloalkyl optionally substitued with one or more R.

In some embodiments of a compound of Formula (IA), A¹ is bicyclic heterocycloalkyl optionally substitued with one or more R.

In some embodiments of a compound of Formula (IA), A¹ is

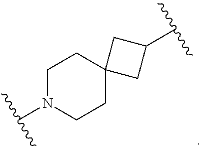 [SMILES: *C1CC2(CCN(*)CC2)C1]

In some embodiments of a compound of Formula (IA), T¹ is absent or C₁-C₃alkylene.

In some embodiments of a compound of Formula (IA), T¹ is absent.

In some embodiments of a compound of Formula (IA), B¹ is C₁-C₁₀alkylene or cycloalkyl, wherein the alkylene and cycloalkyl is optionally substitued with one or more R, and wherein one, two, three, or four carbon atoms in the C₁-C₁₀alkylene are optionally replaced with —S—, —S(═O)—, —S(═O)₂—, —O—, or —NR¹¹—.

In some embodiments of a compound of Formula (IA), B¹ is C₁-C₁₀alkylene optionally substitued with one or more R, and wherein one, two, three, or four carbon atoms in the C₁-C₁₀alkylene are optionally replaced with —S—, —S(═O)—, —S(═O)₂—, —O—, or —NR¹¹—.

In some embodiments of a compound of Formula (IA), B¹ is C₁-C₁₀alkylene optionally substitued with one or more R.

In some embodiments of a compound of Formula (IA), B¹ is C₁-C₆alkylene.

In some embodiments of a compound of Formula (IA), B¹ is —CH₂CH₂CH₂—.

In some embodiments of a compound of Formula (IA), B¹ is —CH₂CH₂CH₂CH₂—.

In some embodiments of a compound of Formula (IA), B¹ is cycloalkyl optionally substitued with one or more R.

In some embodiments of a compound of Formula (IA), B¹ is a monocyclic cycloalkyl optionally substitued with one or more R.

In some embodiments of a compound of Formula (IA), B¹ is a bicyclic cycloalkyl optionally substitued with one or more R.

In some embodiments of a compound of Formula (IA), B¹ is bicyclo[1.1.1]pentanyl, bicyclo[2.2.1]heptanyl, bicyclo[2.2.2]octanyl, or bicyclo[2.1.1]hexanyl.

In some embodiments of a compound of Formula (IA), B¹ is bicyclo[1.1.1]pentanyl.

In some embodiments of a compound of Formula (IA), S¹ is absent.

In some embodiments of a compound of Formula (IA),

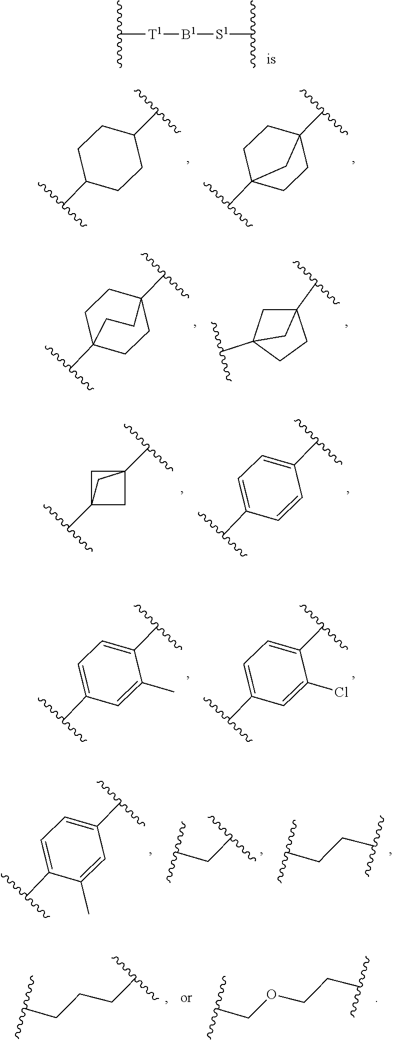

In some embodiments of a compound of Formula (IA),

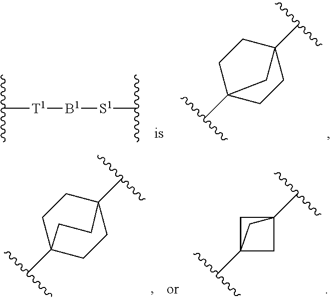

In some embodiments of a compound of Formula (IA),

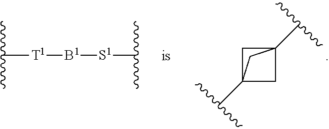

In some embodiments of a compound of Formula (IA), Y¹ is absent, —O—, —S—, —S(═O)₂—, —C(═O)NR¹¹—, or —NR¹¹C(═O)—.

In some embodiments of a compound of Formula (IA), Y¹ is —S(═O)₂—.

In some embodiments of a compound of Formula (IA), Y¹ is —C(═O)NR¹¹—.

In some embodiments of a compound of Formula (IA),

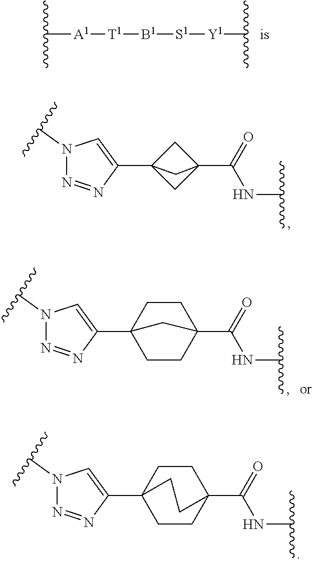

In some embodiments of a compound of Formula (IA),

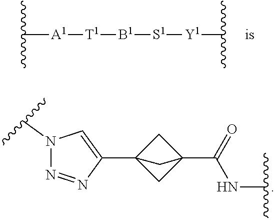

In some embodiments of a compound of Formula (IA), Ring C¹ is aryl or heteroaryl. In some embodiments of a compound of Formula (IA), Ring C¹ is phenyl or heteroaryl. In some embodiments of a compound of Formula (IA), Ring C¹ is heteroaryl. In some embodiments of a compound of Formula (IA), Ring C¹ is 5- to 12-membered heteroaryl. In some embodiments of a compound of Formula (IA), Ring C¹ is 5- to 10-membered heteroaryl. In some embodiments of a compound of Formula (IA), Ring C¹ is 5- to 6-membered heteroaryl. In some embodiments of a compound of Formula (IA), Ring C¹ is 7- to 10-membered heteroaryl. In some embodiments of a compound of Formula (IA), Ring C¹ is monocyclic heteroaryl. In some embodiments of a compound of Formula (IA), Ring C¹ thiadiazolyl or phenyl. In some embodiments of a compound of Formula (IA), Ring C¹ thiadiazolyl. In some embodiments of a compound of Formula (IA), Ring C¹ phenyl.

In some embodiments of a compound of Formula (IA), Ring C¹ is bicyclic heteroaryl.

In some embodiments of a compound of Formula (IA), Ring C¹ is benzothiazolyl or imidazothiadiazolyl.

In some embodiments of a compound of Formula (IA),

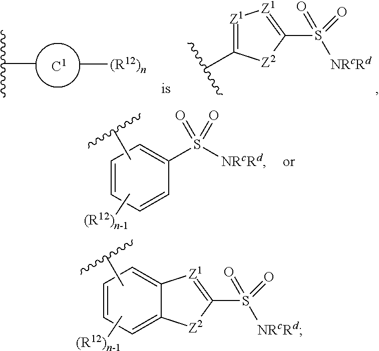

wherein each Z¹ is independently —N— or —CR¹²′—;
Z² is —O—, —S—, or —NR¹²″—;
each R¹²′ is independently hydrogen, halogen, —CN, —OH, —OR^{a}, —NR^{c}R^{d}, C₁-C₆alkyl, C₁-C₆haloalkyl, cycloalkyl, or heterocycloalkyl; and
R¹²″ is hydrogen, —S(═O)R^{a}, —S(═O)₂R^{a}, —S(═O)₂NR^{c}R^{d}, —C(═O)R^{a}, —C(═O)OR^{b}, —C(═O)NR^{c}R^{d}, C₁-C₆alkyl, C₁-C₆haloalkyl, cycloalkyl, or heterocycloalkyl.

In some embodiments, Z² is —O— or —S—. In some embodiments, Z² is —S—. In some embodiments, each Z¹ is —N—.

In some embodiments of a compound of Formula (IA),

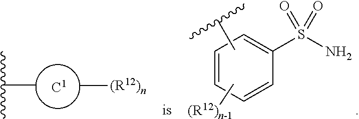

In some embodiments of a compound of Formula (IA),

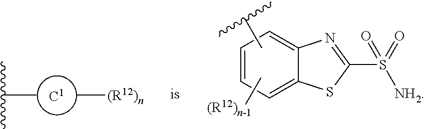

In some embodiments of a compound of Formula (IA), each R¹² is independently halogen, —CN, —OH, —OR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, or C₁-C₆haloalkyl. In some embodiments of a compound of Formula (IA), one of R¹² is —S(═O)₂NR^{c}R^{d}.

In some embodiments of a compound of Formula (IA), each R¹² is independently halogen, —CN, —OH, —OR^{a}, —S(═O)R^{a}, —S(═O)₂R^{a}, —NR^{c}R^{d}, —C(═O)R^{a}, —C(═O)OR^{b}, —C(═O)NR^{c}R^{d}, C₁-C₆alkyl, or C₁-C₆haloalkyl.

In some embodiments of a compound of Formula (IA), each R¹² is independently halogen, —S(═O)₂NR^{c}R^{d}, or —NR^{c}R^{d}.

In some embodiments of a compound of Formula (IA), each R¹² is independently halogen or —NR^{c}R^{d}.

In some embodiments of a compound of Formula (IA), n is 0, 1, 2, 3, 4, or 5. In some embodiments of a compound of Formula (IA), n is 0, 1, 2, 3, or 4. In some embodiments of a compound of Formula (IA), n is 0, 1, 2, or 3. In some embodiments of a compound of Formula (IA), n is 0, 1, or 2. In some embodiments of a compound of Formula (IA), n is 0 or 1.

In some embodiments of a compound of Formula (IA),

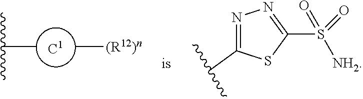

In some embodiments of a compound of Formula (IA),

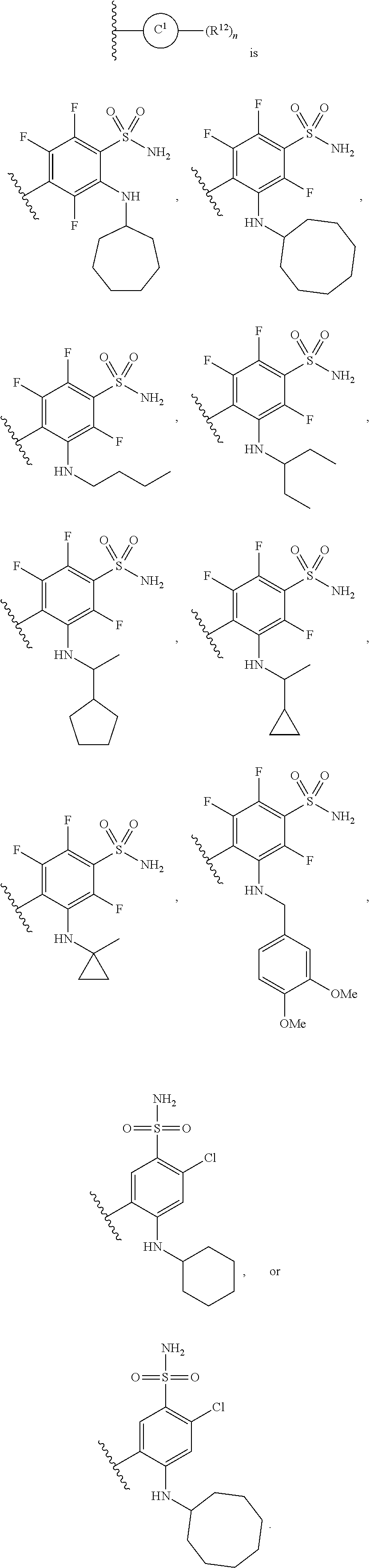

In some embodiments of a compound of Formula (IA),

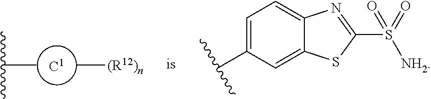

In some embodiments of a compound of Formula (IA),

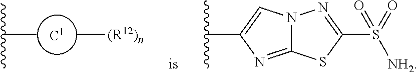

In some embodiments of a compound of Formula (IA), R¹¹ is hydrogen or C₁-C₆alkyl. In some embodiments of a compound of Formula (IIA), R¹¹ is hydrogen.

In some embodiments of a compound of Formula (IA), each R^{a} is independently C₁-C₆alkyl;

In some embodiments of a compound of Formula (IA), each R^{b} is independently hydrogen or C₁-C₆alkyl;

In some embodiments of a compound of Formula (IA), R^{c} and R^{d} are each independently hydrogen, C₁-C₆alkyl, -L-cycloalkyl, or -L-aryl, wherein each alkyl, cycloalkyl, and aryl is independently optionally substituted with one or more R.

In some embodiments of a compound of Formula (IA), L is absent or C₁-C₃alkylene. In some embodiments of a compound of Formula (IA), L is absent. In some embodiments of a compound of Formula (IA), L is C₁-C₃alkylene.

In some embodiments of a compound of Formula (IA), each R is independently C₁-C₃alkyl or C₁-C₃alkoxy.

In some embodiments of a compound of Formula (A), (AC), (A′) or (AC′), TL₁ is of Formula (IA):

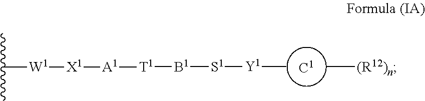

wherein

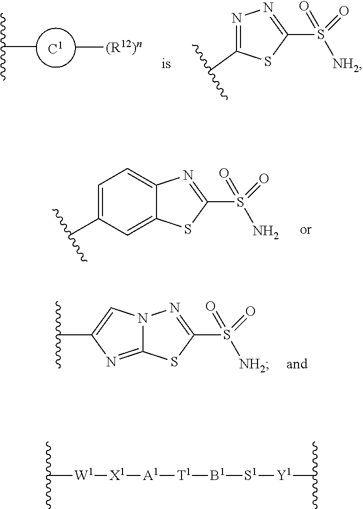

are taken together to form a linking group that comprises 3 to 30 atoms (optionally, 5-25 atoms, 8-20 atoms, 8-15 atoms, or 10-14 atoms, or 8, 9, 10, 11, 12, 13, 14, or 15 atoms) in the linear chain.

In some embodiments,

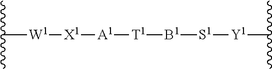

are taken together to form a linking group that comprises 3 to 30 atoms in the linear chain, which are selected from C, N, and O. In some embodiments, the linking group comprises 3 to 30 atoms in the linear chain, including carbon, 1-5 N and 0-1 O. In some embodiments, the linking group comprises 3 to 30 atoms in the linear chain selected from N and C, including 2, 3, or 4 N. In some embodiments, the linking group comprises one or more cyclic groups, and the atoms in the linear chain comprises a part of the cyclic groups. In some embodiments, both A¹ and B¹ are cyclic groups. In some embodiments, the linking group comprises one or more groups selected from —O—, —S—, —S(═O)—, —S(═O)₂—, —NR²¹—, —C(═O)—, —C(═O)NR²¹—, —NR²¹C(═O)—, —S(═O)₂NR²¹—, or —NR²¹S(═O)₂, optionally substituted C₁-C₁₀alkylene, optionally substituted C₁-C₁₀heteroalkylene, optionally substituted cycloalkyl, optionally substituted heterocycloalkyl, optionally substituted aryl, or optionally substituted heteroaryl, or a combination thereof.

In some embodiments of a compound of Formula (A), (AC), (A′) or (AC′), TL₂ is of Formula (IIA):

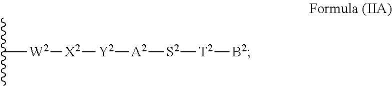

wherein:
W² is absent, —O—, —S—, —S(═O)—, —S(═O)₂—, —NR²¹—, —C(═O)—, —C(═O)NR²¹—, —NR²¹C(═O)—, —S(═O)₂NR²¹—, or —NR²¹S(═O)₂;
X² is absent or C₁-C₁₀alkylene optionally substituted with one or more R, and wherein one, two, three, or four carbon atoms of the C₁-C₁₀alkylene are optionally replaced with —S—, —S(═O)—, —S(═O)₂—, —O—, or —NR²¹—;
Y² is absent, —O—, —S—, —S(═O)—, —S(═O)₂—, —NR²¹—, —C(═O)—, —C(═O)NR²¹—, —NR²¹C(═O)—, —S(═O)₂NR²¹—, or —NR²¹S(═O)₂;
A² is absent, C₁-C₁₀alkylene, cycloalkyl, heterocycloalkyl, aryl, or heteroaryl, wherein the alkylene, cycloalkyl, heterocycloalkyl, aryl, and heteroaryl is optionally substitued with one or more R²²;
each R²² is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
S² is absent, —O—, —S—, —S(═O)—, —S(═O)₂—, —NR²¹—, —C(═O)—, —C(═O)NR²¹—, —NR²¹C(═O)—, —S(═O)₂NR²¹—, or —NR²¹S(═O)₂;
T² is absent or C₁-C₁₀alkylene optionally substituted with one or more R, and wherein one, two, three, or four carbon atoms of the C₁-C₁₀alkylene are optionally replaced with —S—, —S(═O)—, —S(═O)₂—, —O—, or —NR²¹—;
R²¹ is hydrogen, C₁-C₆alkyl, C₁-C₆haloalkyl, C₁-C₆hydroxyalkyl, C₁-C₆aminoalkyl, or C₁-C₆heteroalkyl;
(i) B² is absent;
or
(ii) B² is —N(R²³)₂;
each R²³ is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
   wherein each alkyl, alkenyl, alkynyl, cycloalkyl, heterocycloalkyl, aryl, and heteroaryl is optionally and independently substituted with one or more R;
or
(iii) B² is —CF₃;
or
(iv) B² is cycloalkyl, heterocycloalkyl, aryl, or heteroaryl; wherein the cycloalkyl, heterocycloalkyl, aryl, and heteroaryl is optionally substitued with one or more R²⁴;
each R²⁴ is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
or
(v) B² is

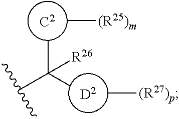

Ring C² is cycloalkyl, heterocycloalkyl, aryl, or heteroaryl;
each R²⁵ is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
m is 0, 1, 2, 3, or 4;
R²⁶ is hydrogen, C₁-C₆alkyl, C₁-C₆haloalkyl, C₁-C₆hydroxyalkyl, C₁-C₆aminoalkyl, or C₁-C₆heteroalkyl;
Ring D² is cycloalkyl, heterocycloalkyl, aryl, or heteroaryl;
each R²⁷ is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
p is 0, 1, 2, 3, or 4;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, -L-cycloalkyl, -L-heterocycloalkyl, -L-aryl, or -L-heteroaryl, wherein each alkyl, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, -L-cycloalkyl, -L-heterocycloalkyl, -L-aryl, or -L-heteroaryl, wherein each alkyl, alkenyl, alkynyl, cycloalkyl, heterocycloalkyl, aryl, and heteroaryl is independently optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, -L-cycloalkyl, -L-heterocycloalkyl, -L-aryl, or -L-heteroaryl, wherein each alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
L is absent or C₁-C₃alkylene optionally substituted with one or more R; and
each R is independently halogen, —CN, —OH, —SF₅, —SH, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, C₃-C₆cycloalkyl, or 3- to 6-membered heterocycloalkyl;
or two R on the same atom form an oxo;
or two R on the same or different atoms are taken together to form a cycloalkyl, heterocycloalkyl, aryl, or heteroaryl.

In some embodiments of a compound of Formula (A), (AC), (A′) or (AC′), TL₂ is of Formula (IIA):

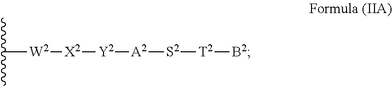

wherein:
W² is absent, —C(═O)—, or —NR²¹C(═O)—;
X² is C₁-C₁₀alkylene optionally substituted with one or more R;
Y² is absent, —NR²¹—, or —NR²¹C(═O)—;
A² is C₁-C₁₀alkylene, cycloalkyl, heterocycloalkyl, aryl, or heteroaryl, wherein the alkylene, cycloalkyl, heterocycloalkyl, aryl, and heteroaryl is optionally substitued with one or more R²²;
each R²² is independently halogen, —CN, —C(═O)OR^{b}, —C(═O)NR^{c}R^{d}, C₁-C₆alkyl, C₁-C₆haloalkyl, or C₁-C₆aminoalkyl;
S² is absent, —NR²¹—, —C(═O)—, or —NR²¹C(═O)—;
T² is absent or C₁-C₁₀alkylene;
R²¹ is hydrogen;
B² is cycloalkyl, heterocycloalkyl, aryl, or heteroaryl; wherein the cycloalkyl, heterocycloalkyl, aryl, and heteroaryl is optionally substitued with one or more R²⁴;
   each R²⁴ is independently halogen, —OH, —SF₅, C₁-C₆alkyl, C₁-C₆haloalkyl, cycloalkyl, or heterocycloalkyl;
or B² is

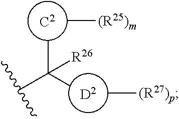

Ring C² is aryl;
each R²⁵ is independently halogen, —OH, or C₁-C₆alkyl;
m is 0, 1, or 2;
R²⁶ is hydrogen or C₁-C₆alkyl;
Ring D² is aryl;
each R²⁷ is independently halogen, —OH, or C₁-C₆alkyl;
p is 0, 1, or 2;
each R^{b} is independently hydrogen or C₁-C₆alkyl;
R^{c} and R^{d} are each independently hydrogen or C₁-C₆alkyl; and
each R is independently —C(═O)OH or —C(═O)NH₂.

In some embodiments of a compound of Formula (IIA), W² is absent, —C(═O)—, or —NR²¹C(═O)—.

In some embodiments of a compound of Formula (IIA), W² is —C(═O)—. In some embodiments of a compound of Formula (IIA), W² is absent. In some embodiments of a compound of Formula (IIA), W² is —C(═O)—. In some embodiments of a compound of Formula (IIA), W² is —NR²¹C(═O)—.

In some embodiments of a compound of Formula (IIA), X² is C₁-C₆alkylene optionally substituted with one or more R.

In some embodiments of a compound of Formula (IIA), X² is C₁-C₄alkylene optionally substituted with one or more R.

In some embodiments of a compound of Formula (IIA), X² is —CH₂—.

In some embodiments of a compound of Formula (IIA), X² is —CH₂CH[C(═O)OH]— or —CH₂CH[C(═O)NH₂]—.

In some embodiments of a compound of Formula (IIA), X² is —CH₂CH₂CH[(C—O)OH]— or —CH₂CH₂CH[C(═O)NH₂]—.

In some embodiments of a compound of Formula (IIA), X² is optionally substituted with one or more R, and wherein one or two carbon atoms of the C₁-C₁₀alkylene are replaced with —S—, —S(═O)—, —S(═O)₂—, —O—, or —NR²¹—

In some embodiments of a compound of Formula (IIA), Y² is absent, —O—, —S—, —NR²¹—, —C(═O)—, —C(═O)NR²¹—, or —NR²¹C(═O)—.

In some embodiments of a compound of Formula (IIA), Y² is absent, —NR²¹—, or —NR²¹C(═O)—.

In some embodiments of a compound of Formula (IIA), Y² is —NR²¹C(═O)—.

In some embodiments of a compound of Formula (IIA),

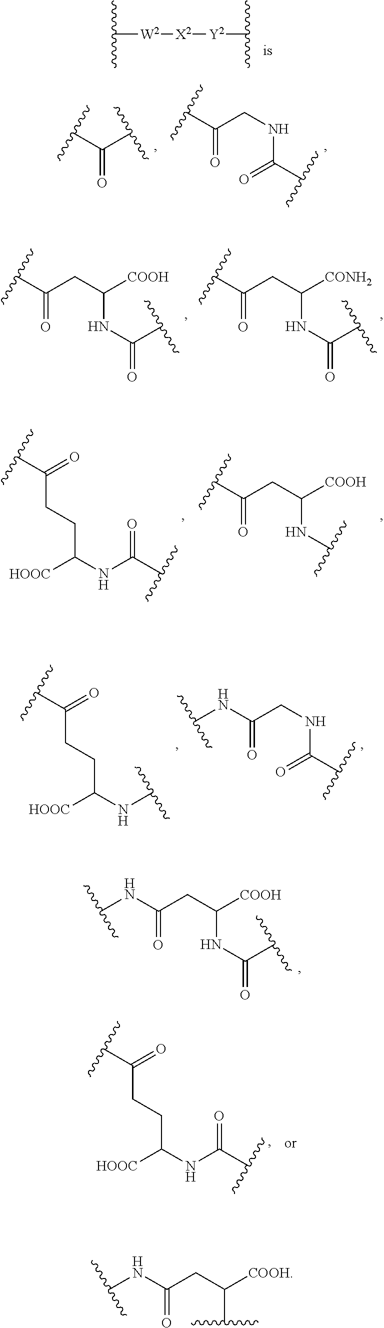

In some embodiments of a compound of Formula (IIA),

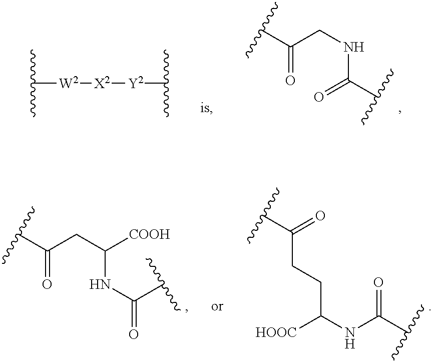

In some embodiments of a compound of Formula (IIA), A² is C₁-C₁₀alkylene, heterocycloalkyl, aryl, or heteroaryl, wherein the alkylene, heterocycloalkyl, aryl, and heteroaryl is optionally substitued with one or more R²².

In some embodiments of a compound of Formula (IIA), A² is C₁-C₆alkylene optionally substitued with one or more R²².

In some embodiments of a compound of Formula (IIA), A² is —CH₂CH[C(═O)OH]—.

In some embodiments of a compound of Formula (IIA), A² is heterocycloalkyl optionally substitued with one or more R²².

In some embodiments of a compound of Formula (IIA), A² is pyrrolidinyl, piperidinyl, piperazinyl, or morpholinyl, each optionally substitued with one or more R²².

In some embodiments of a compound of Formula (IIA), A² is piperidinyl optionally substitued with one or more R²².

In some embodiments of a compound of Formula (IIA), A² is

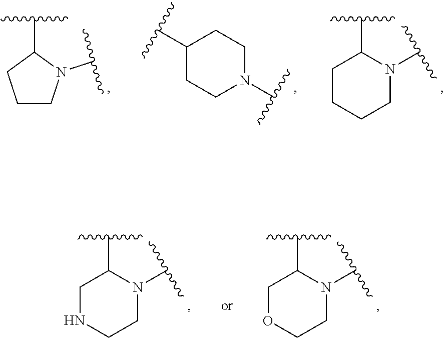 [SMILES: *C1CCCN1* ; *C1CCN(*)CC1 ; *C1CCCCN1* ; *C1CNCCN1* ; *C1COCCN1*]

each optionally substitued with one or more R²².

In some embodiments of a compound of Formula (IIA), A² is

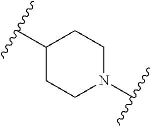 [SMILES: *C1CCN(*)CC1]

optionally substitued with one or more R²².

In some embodiments of a compound of Formula (IIA), A² is aryl or heteroaryl, wherein the aryl and heteroaryl is optionally substitued with one or more R²².

In some embodiments of a compound of Formula (IIA), A² is phenyl, pyridinyl, pyrimidinyl, or pyrazinyl, each optionally substitued with one or more R²².

In some embodiments of a compound of Formula (IIA), A² is

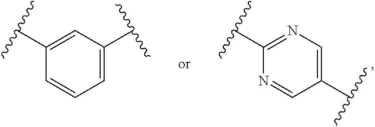 [SMILES: *c1cccc(*)c1 ; *c1cnc(*)nc1]

each optionally substitued with one or more R²².

In some embodiments of a compound of Formula (IIA), A² is

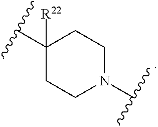

In some embodiments of a compound of Formula (IIA), each R²² is independently halogen, —CN, —OH, —OR^{a}, —NR^{c}R^{d}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl.

In some embodiments of a compound of Formula (IIA), each R²² is independently —CN, —C(═O)OR^{b}, —C(═O)NR^{c}R^{d}, C₁-C₆alkyl, or C₁-C₆aminoalkyl.

In some embodiments of a compound of Formula (IIA), each R²² is independently —C(═O)OR^{b} or —C(═O)NR^{c}R^{d}.

In some embodiments of a compound of Formula (IIA), each R²² is independently —C(═O)NR^{c}R^{d}.

In some embodiments of a compound of Formula (IIA), A² is

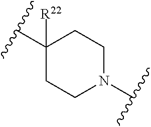

and R²² is halogen, —CN, —OH, —OR^{a}, —NR^{c}R^{d}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl.

In some embodiments of a compound of Formula (IIA), A² is

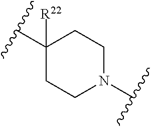

and R²² is —CN, —C(═O)OR^{b}, —C(═O)NR^{c}R^{d}, C₁-C₆alkyl, or C₁-C₆aminoalkyl.

In some embodiments of a compound of Formula (IIA), A² is

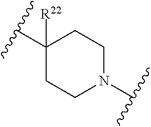

and R²² is —C(═O)OR^{b} or —C(═O)NR^{c}R^{d}.

In some embodiments of a compound of Formula (IIA), A² is

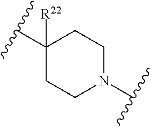

and R²² is —C(═O)NR^{c}R^{d}.

In some embodiments of a compound of Formula (IIA), A² is

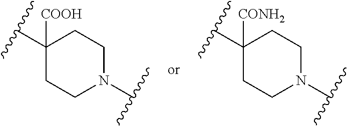 [SMILES: *N1CCC(*)(C(=O)O)CC1 ; *N1CCC(*)(C(N)=O)CC1]

In some embodiments of a compound of Formula (IIA), A² is

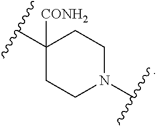 [SMILES: *N1CCC(*)(C(N)=O)CC1]

In some embodiments of a compound of Formula (IIA), S² is absent, —O—, —S—, —NR²¹—, —C(═O)—, —C(═O)NR²¹—, or —NR²¹C(═O)—.

In some embodiments of a compound of Formula (IIA), S² is absent, —NR²¹—, —C(═O)—, or —NR²¹C(═O)—.

In some embodiments of a compound of Formula (IIA), S² is —C(═O)— or —NR²¹C(═O)—.

In some embodiments of a compound of Formula (IIA), T² is absent or C₁-C₆alkylene optionally substituted with one or more R.

In some embodiments of a compound of Formula (IIA), T² is C₁-C₆alkylene optionally substituted with one or more R.

In some embodiments of a compound of Formula (IIA), T² is —CH₂CH₂—.

In some embodiments of a compound of Formula (IIA), T² is —CH₂CH₂CH₂—.

In some embodiments of a compound of Formula (IIA),

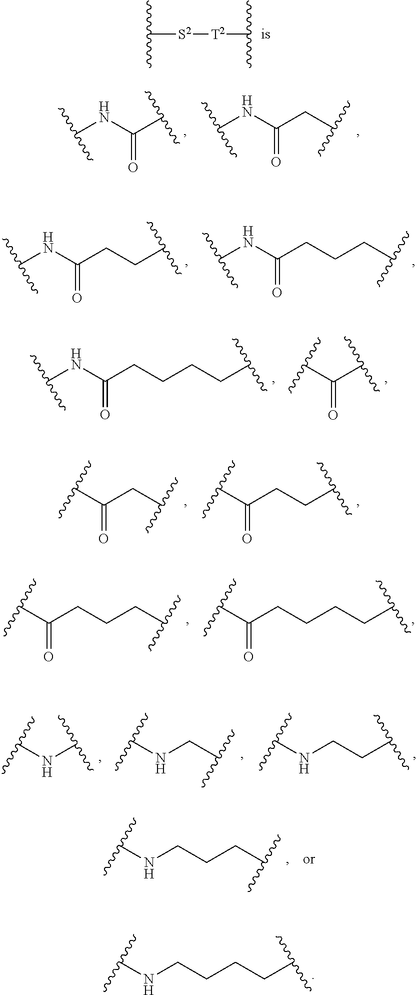

In some embodiments of a compound of Formula (IIA),

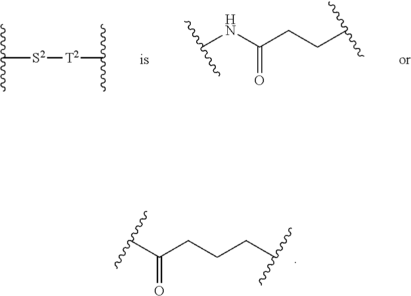

In some embodiments of a compound of Formula (IIA), B² is absent.

In some embodiments of a compound of Formula (IIA), B² is —N(R²³)₂.

In some embodiments of a compound of Formula (IIA), B² is —CF₃.

In some embodiments of a compound of Formula (IIA), B² is cycloalkyl, heterocycloalkyl, aryl, or heteroaryl; wherein the cycloalkyl, heterocycloalkyl, aryl, and heteroaryl is optionally substitued with one or more R²⁴.

In some embodiments of a compound of Formula (IIA), B² is cycloalkyl optionally substitued with one or more R²⁴.

In some embodiments of a compound of Formula (IIA), B² is monocyclic cycloalkyl optionally substitued with one or more R²⁴.

In some embodiments of a compound of Formula (IIA), B² is bicyclic cycloalkyl optionally substitued with one or more R²⁴.

In some embodiments of a compound of Formula (IIA), B² is phenyl optionally substitued with one or more R²⁴.

In some embodiments of a compound of Formula (IIA), B² is heteroaryl optionally substitued with one or more R²⁴.

In some embodiments of a compound of Formula (IIA), B² is 5- to 10-membered heteroaryl (e.g., monocyclic or bicyclic heteroaryl) optionally substitued with one or more R²⁴.

In some embodiments of a compound of Formula (IIA), B² is 5- to 6-membered heteroaryl optionally substitued with one or more R²⁴.

In some embodiments of a compound of Formula (IIA), B² is indazolyl, phenyl, pyridyl, pyridininyl, pyrazinyl, pyridazinyl, cyclopropyl, cyclohexyl, cyclobutyl, cycloheptyl, pyrazolyl, oxazolyl, triazolyl, spiroheptanyl, or bicycloheptanyl.

In some embodiments of a compound of Formula (IIA), each R²⁴ is independently halogen, —CN, —OH, —OR^{a}, —NR^{c}R^{d}, —SF₅, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; wherein each alkyl, cycloalkyl, heterocycloalkyl, aryl, and heteroaryl is optionally and independently substituted with one or more R.

In some embodiments of a compound of Formula (IIA), each R²⁴ is independently halogen, —OH, C₁-C₆alkyl, or C₁-C₆haloalkyl.

In some embodiments of a compound of Formula (IIA), B² is

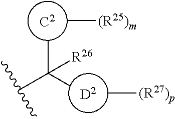

wherein:
Ring C² is cycloalkyl, heterocycloalkyl, aryl, or heteroaryl;
each R²⁵ is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
m is 0, 1, 2, 3, or 4;
R²⁶ is hydrogen, C₁-C₆alkyl, C₁-C₆haloalkyl, C₁-C₆hydroxyalkyl, C₁-C₆aminoalkyl, or C₁-C₆heteroalkyl;
Ring D² is cycloalkyl, heterocycloalkyl, aryl, or heteroaryl;
each R²⁷ is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
p is 0, 1, 2, 3, or 4.

In some embodiments of a compound of Formula (IIA), Ring C² is phenyl.

In some embodiments of a compound of Formula (IIA), each R²⁵ is independently halogen, —CN, —OH, —OR^{a}, —SF₅, —SH, —SR^{a}, —NR^{c}R^{d}, C₁-C₆alkyl, C₁-C₆haloalkyl, or C₁-C₆hydroxyalkyl. In some embodiments of a compound of Formula (IIA), each R²⁵ is independently halogen, —CN, —OH, —OR^{a}, —NR^{c}R^{d}, or C₁-C₆alkyl. In some embodiments of a compound of Formula (IA), each R²⁵ is independently —OH.

In some embodiments of a compound of Formula (IIA), m is 0 or 1. In some embodiments of a compound of Formula (IIA), m is 0, 1, or 2. In some embodiments of a compound of Formula (IIA), m is 0. In some embodiments of a compound of Formula (IIA), m is 1. In some embodiments of a compound of Formula (IIA), m is 2. In some embodiments of a compound of Formula (IIA), m is 3.

In some embodiments of a compound of Formula (IIA), Ring D₂ is phenyl.

In some embodiments of a compound of Formula (IIA), each R²⁷ is independently halogen, —CN, —OH, —OR^{a}, —SF₅, —SH, —SR^{a}, —NR^{c}R^{d}, C₁-C₆alkyl, C₁-C₆haloalkyl, or C₁-C₆hydroxyalkyl. In some embodiments of a compound of Formula (IIA), each R²⁷ is independently halogen, —CN, —OH, —OR^{a}, —NR^{c}R^{d}, or C₁-C₆alkyl. In some embodiments of a compound of Formula (IIA), each R²⁷ is independently —OH.

In some embodiments of a compound of Formula (IIA), p is 0 or 1. In some embodiments of a compound of Formula (IIA), p is 0, 1, or 2. In some embodiments of a compound of Formula (IIA), p is 0. In some embodiments of a compound of Formula (IIA), p is 1. In some embodiments of a compound of Formula (IIA), p is 2. In some embodiments of a compound of Formula (IIA), p is 3.

In some embodiments of a compound of Formula (IIA), R²⁶ is hydrogen or C₁-C₆alkyl. In some embodiments of a compound of Formula (IIA), R²⁶ is C₁-C₆alkyl.

In some embodiments of a compound of Formula (IIA), B² is

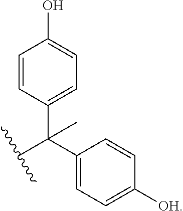 [SMILES: *C(C)(c1ccc(O)cc1)c1ccc(O)cc1]

In some embodiments of a compound of Formula (IIA), R²¹ is hydrogen or C₁-C₆alkyl. In some embodiments of a compound of Formula (IIA), R²¹ is hydrogen.

In some embodiments of a compound of Formula (IIA), each R^{a} is independently C₁-C₆alkyl;

In some embodiments of a compound of Formula (IIA), each R^{b} is independently hydrogen or C₁-C₆alkyl.

In some embodiments of a compound of Formula (IIA), R^{c} and R^{d} are each independently hydrogen or C₁-C₆alkyl.

In some embodiments of a compound of Formula (IIA), each R is independently —C(═O)OH or —C(═O)NH₂.

In some embodiments of a compound of Formula (A), (AC), (A′) or (AC′), TL₂ is of Formula (IIA):

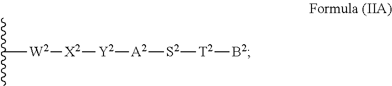

wherein
B² is

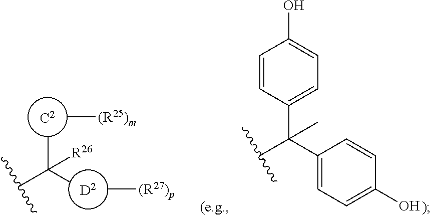

and

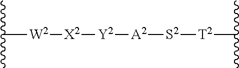

are taken together to form a linking group that comprises 3 to 30 atoms (optionally, 5-25 atoms, 8-20 atoms, 8-15 atoms, or 10-14 atoms, or 8, 9, 10, 11, 12, 13, 14, or 15 atoms) in the linear chain.

In some embodiments,

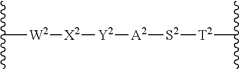

together form a linking group that comprises 3 to 30 atoms in the linear chain, which are selected from C, N, and O. In some embodiments, the linking group comprises 3 to 30 atoms in the linear chain, including carbon, 1-5 N and 0-1 O. In some embodiments, the linking group comprises 3 to 30 atoms in the linear chain selected from N and C, including 2, 3, or 4 N. In some embodiments, the linking group comprises one or more cyclic groups, and the atoms in the linear chain comprises a part of the cyclic groups. In some embodiments, A² is a cyclic group. In some embodiments, the linking group comprises one or more groups selected from —O—, —S—, —S(═O)—, —S(═O)₂—, —NR²¹—, —C(═O)—, —C(═O)NR²¹—, —NR²¹C(═O)—, —S(═O)₂NR²¹—, or —NR²¹S(═O)₂, optionally substituted C₁-C₁₀alkylene, optionally substituted C₁-C₁₀heteroalkylene, optionally substituted cycloalkyl, optionally substituted heterocycloalkyl, optionally substituted aryl, or optionally substituted heteroaryl, or a combination thereof.

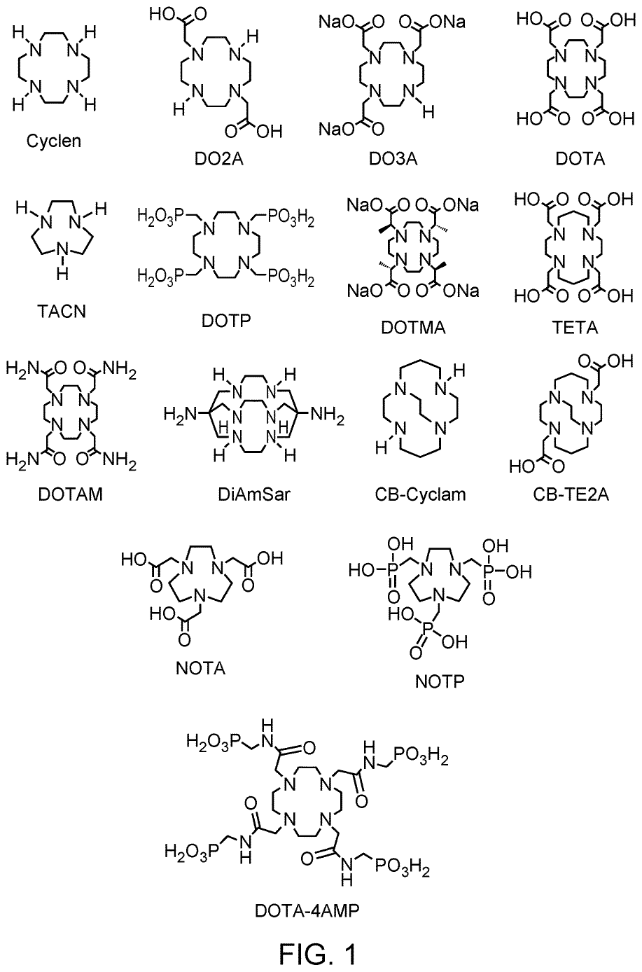

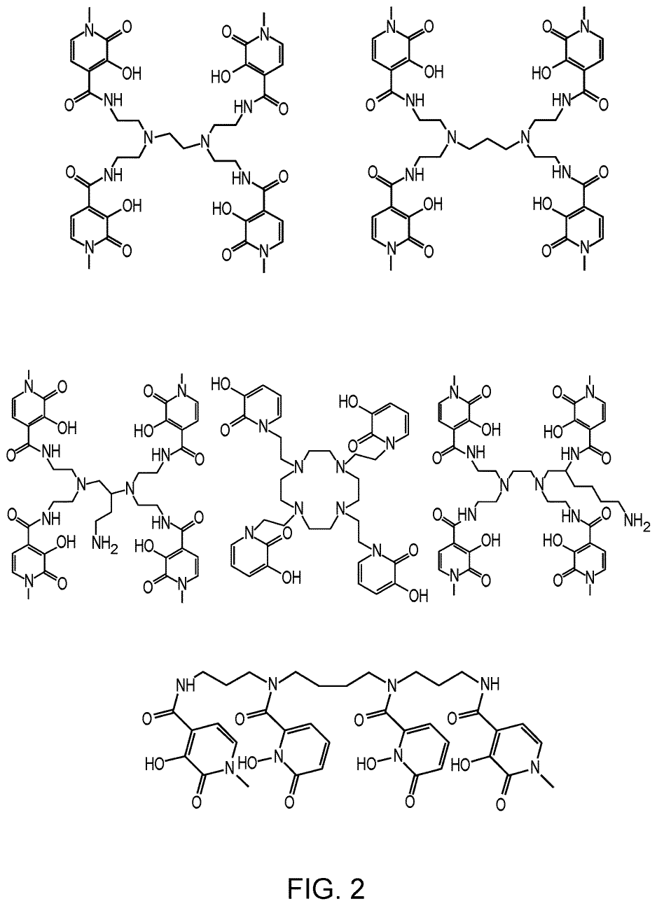

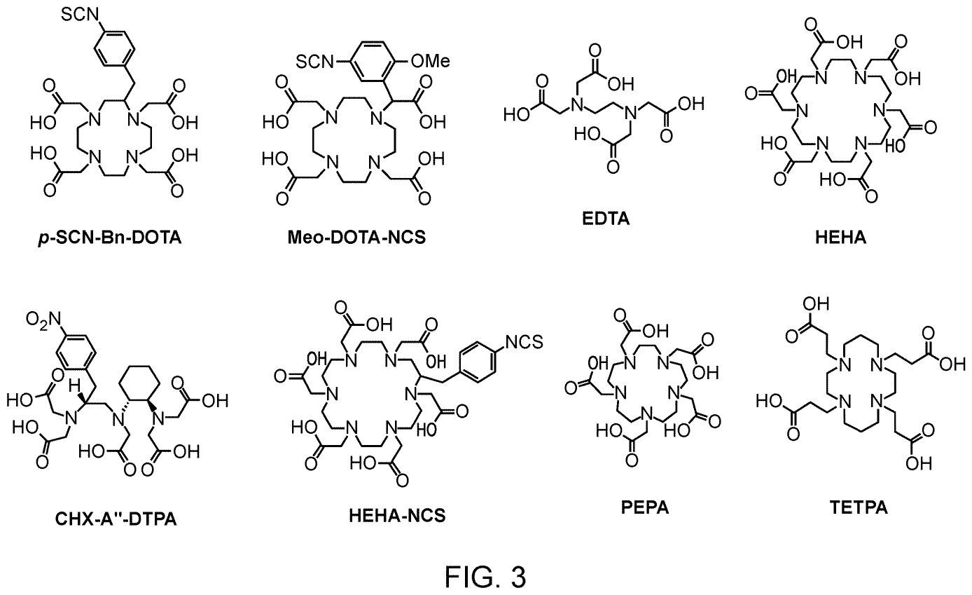

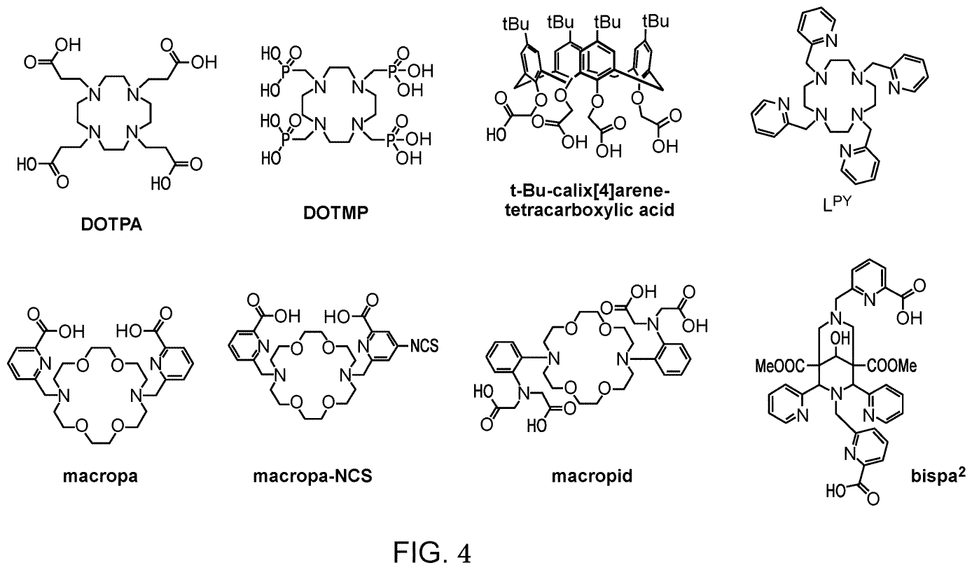

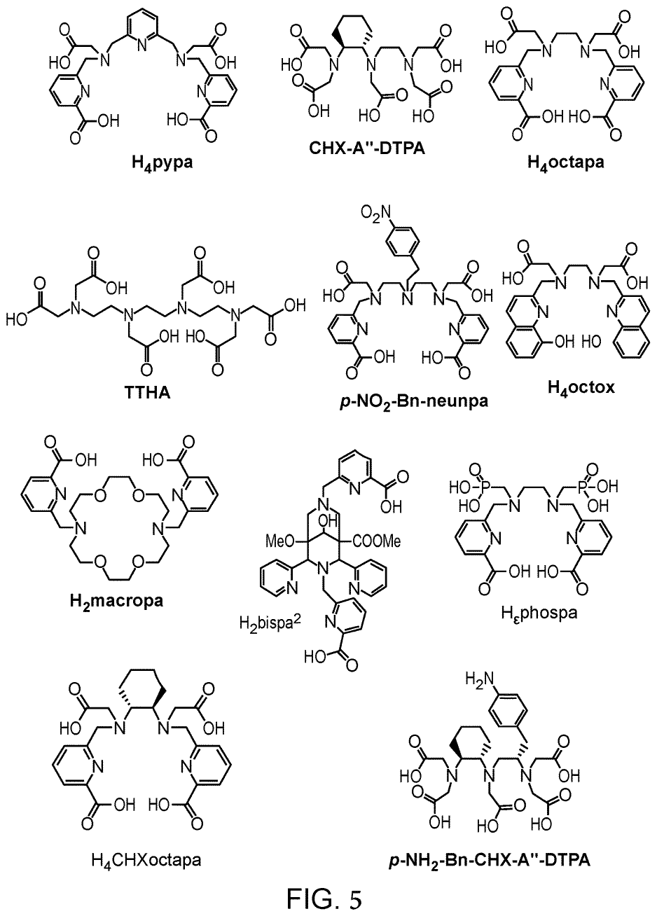

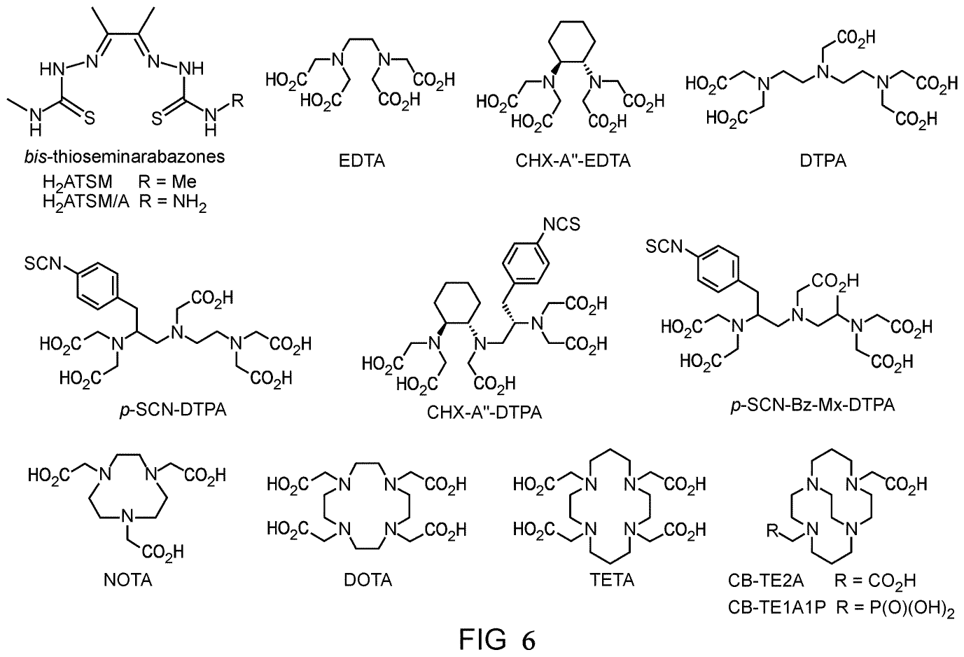

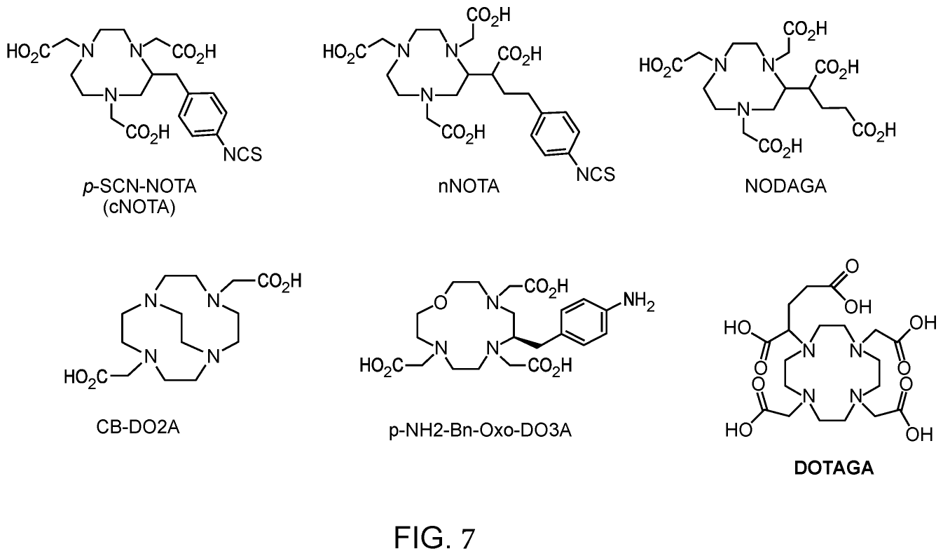

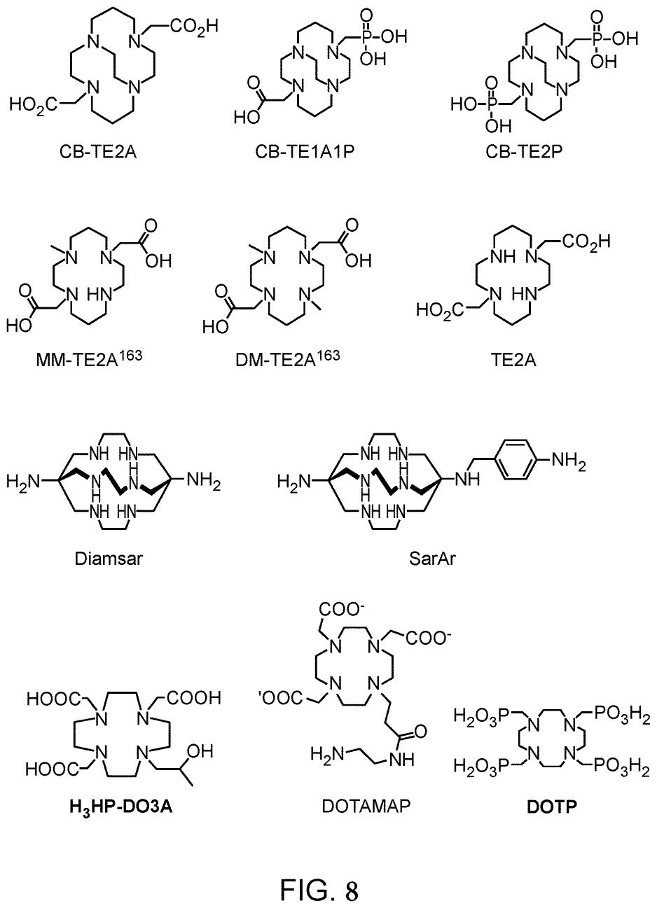

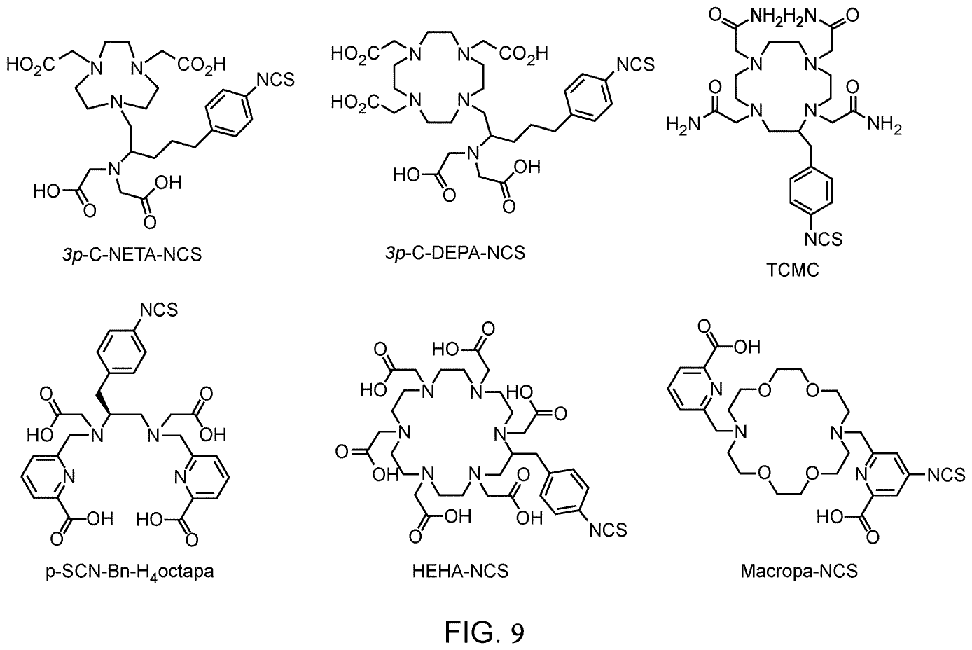

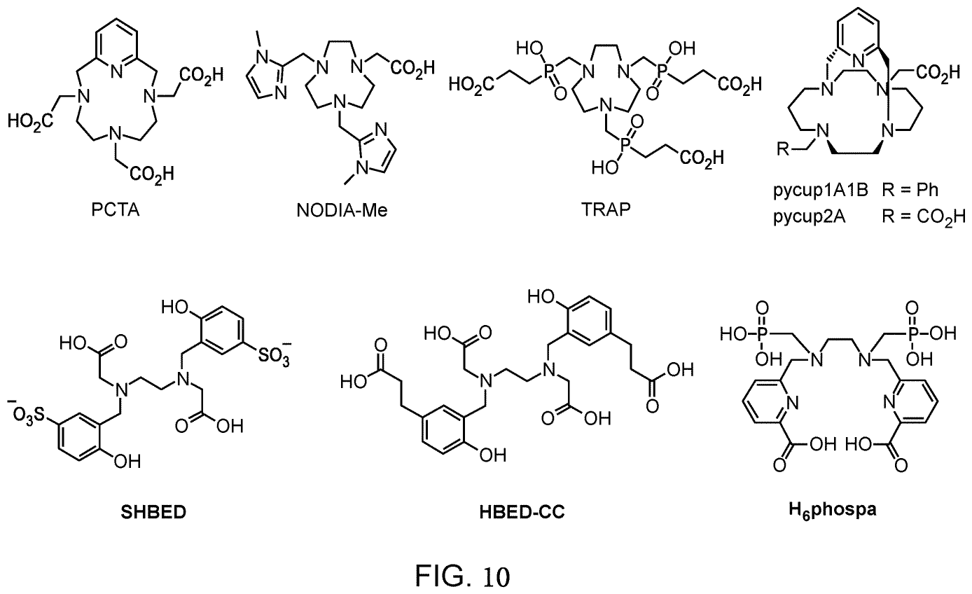

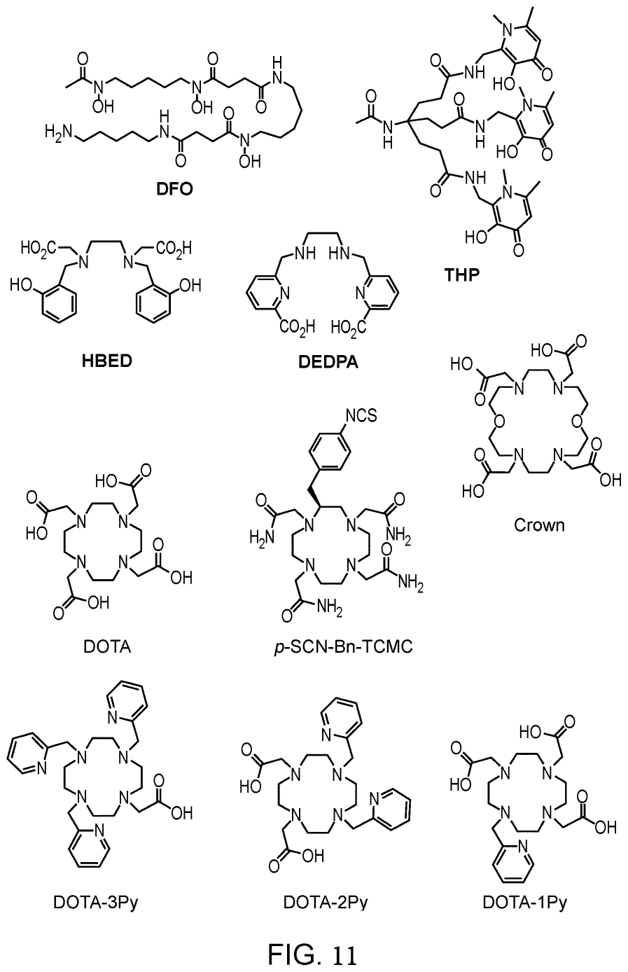

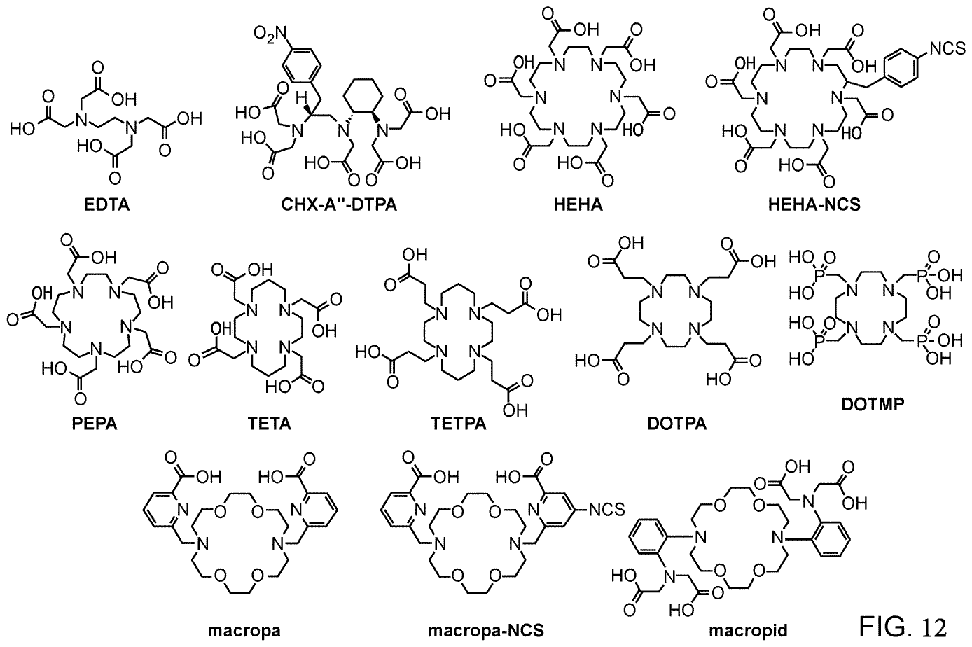

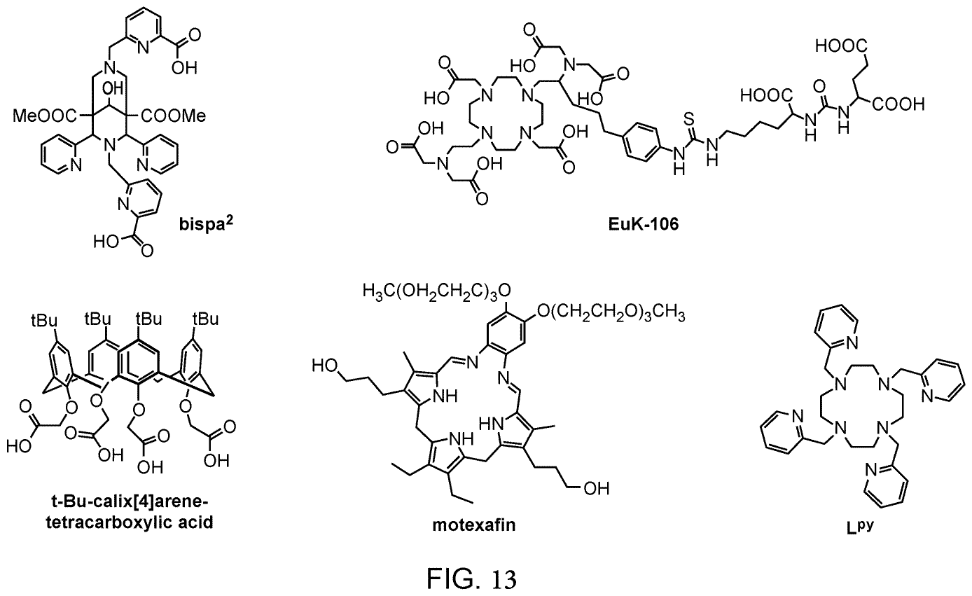

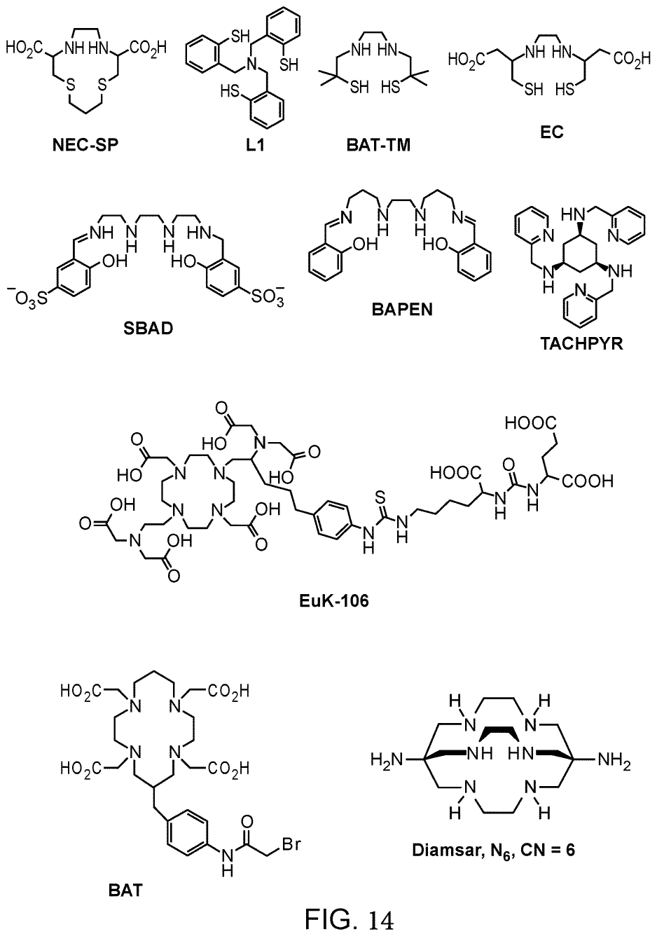

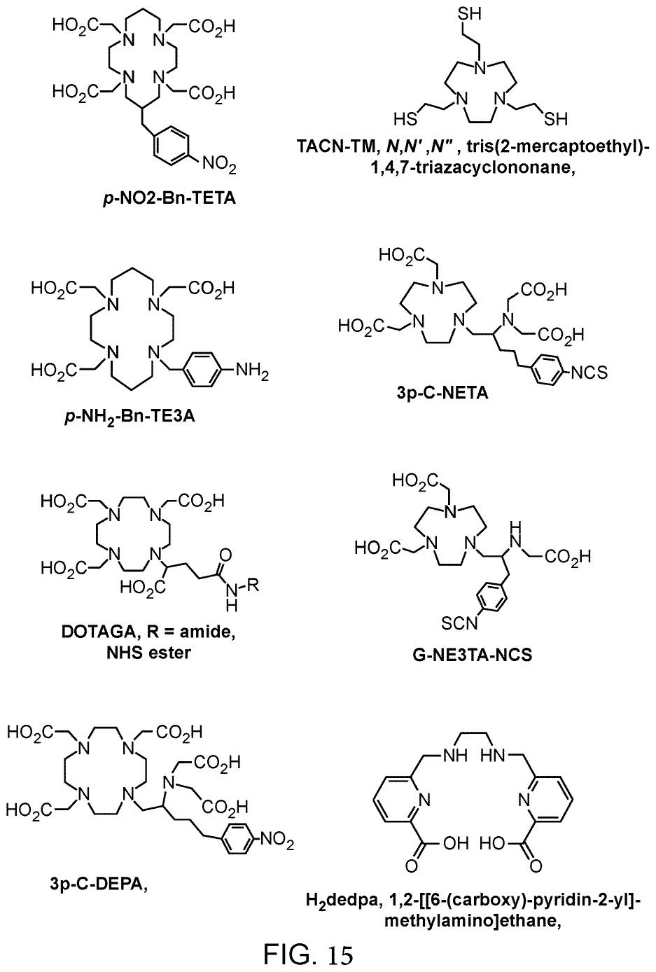

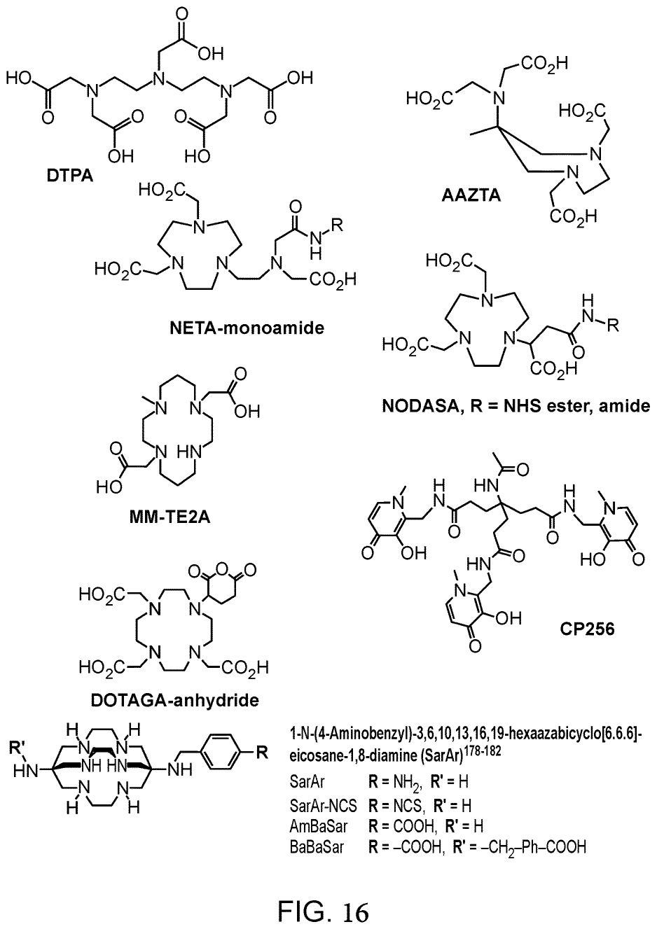

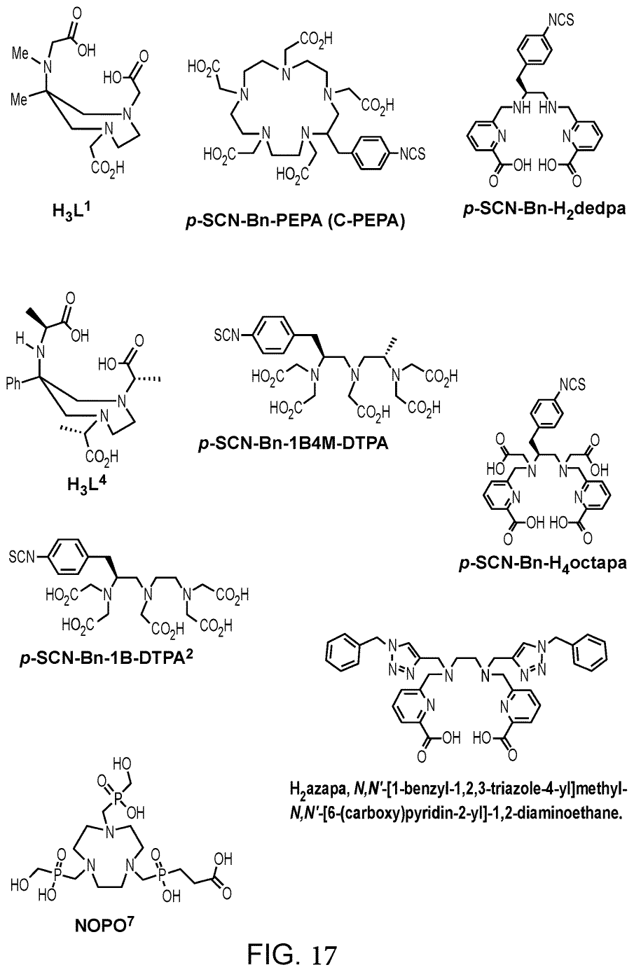

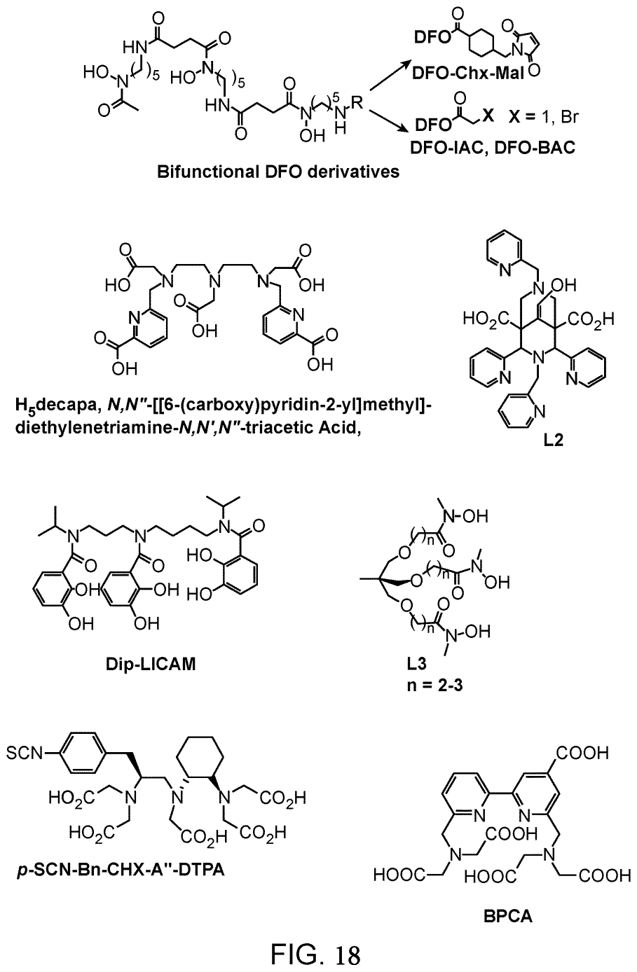

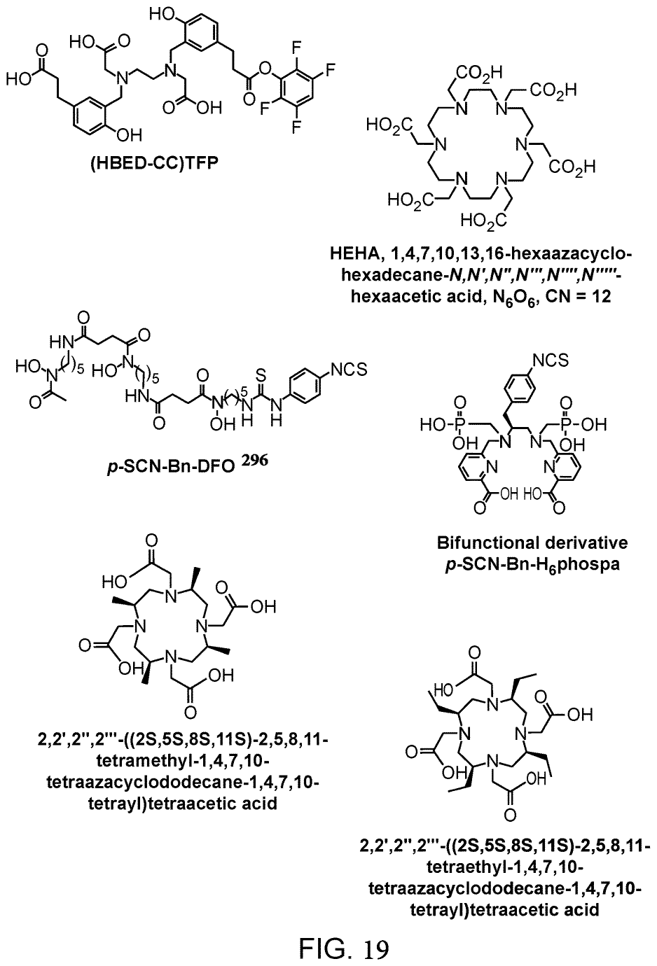

In some embodiments, disclosed herein is radiopharmaceutical conjugate comprising: (i) a radionuclide selected from Table 3 (e.g., 225 Ac, 177Lu, 64Cu, and 68Ga) and (ii) a compound of formula (A), or a salt thereof, wherein the CL is selected from a chelator of FIGS. 1-19 (e.g., DOTA, DOTA-GA); s is 0; ring A is piperidinyl or piperazinyl, each is optionally substituted;

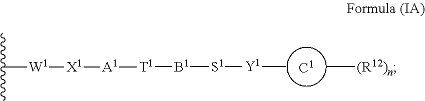

TL₁ is of Formula (IA)
wherein:
W¹ is absent, —C(═O)—, or —C(═O)NR¹¹—; X¹ is C₁-C₁₀alkylene; A¹ is absent or heteroaryl (.e.g, triazolyl) optionally substitued with one or more R; T¹ is absent; B¹ is C₁-C₁₀alkylene or cycloalkyl (e . . . g, bicyclo[1.1.1]pentanyl); S¹ is absent; Y¹ is absent, —O—, —S—, —S(═O)₂—, —C(═O)NR¹¹—, or —NR¹¹C(═O)—; R¹¹ is hydrogen; Ring C¹ is heteroaryl; each R¹² is independently halogen, —S(═O)₂NR^{c}R^{d}, or —NR^{c}R^{d}; and n is 0, 1, 2, or 3; and
TL₂ is of Formula (IIA):

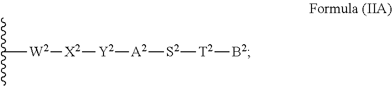

wherein:
W² is absent, —C(═O)—, or —NR²¹C(═O)—; X² is C₁-C₁₀alkylene optionally substituted with one or more R; Y² is absent, —NR²¹—, or —NR²¹C(═O)—; A² is C₁-C₁₀alkylene, cycloalkyl, heterocycloalkyl, aryl, or heteroaryl, wherein the alkylene, cycloalkyl, heterocycloalkyl, aryl, and heteroaryl is optionally substitued with one or more R²²; each R²² is independently halogen, —CN, —C(═O)OR^{b}, —C(═O)NR^{c}R^{d}, C₁-C₆alkyl, or C₁-C₆haloalkyl; S² is absent, —NR²¹—, —C(═O)—, or —NR²¹C(═O)—; T² is absent or C₁-C₁₀alkylene optionally substituted with one or more R, and wherein one, two, three, or four carbon atoms of the C₁-C₁₀alkylene are optionally replaced with —S—, —S(═O)—, —S(═O)₂—, —O—, or —NR²¹—; R²¹ is hydrogen, C₁-C₆alkyl, C₁-C₆haloalkyl, C₁-C₆hydroxyalkyl, C₁-C₆aminoalkyl, or C₁-C₆heteroalkyl; B² is

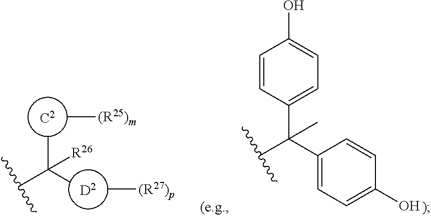

Ring C² is aryl; each R²⁵ is independently halogen, —CN, —OH, —OR^{a}, —NR^{c}R^{d}, C₁-C₆alkyl, or C₁-C₆haloalkyl; m is 0, 1, or 2; R²⁶ is hydrogen, C₁-C₆alkyl, or C₁-C₆haloalkyl; Ring D² is aryl; each R²⁷ is independently halogen, —CN, —OH, —OR^{a}, —NR^{c}R^{d}, C₁-C₆alkyl, or C₁-C₆haloalkyl; p is 0, 1, or 2.

Also disclosed herein is a conjugate of Formula (B), or a pharmaceutically acceptable salt or stereoisomer thereof:

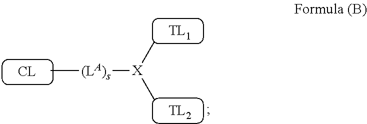

wherein:
   L^{A} represents a linker;
   s is 0 or 1;
   CL represents a metal chelator;
   X is N, optionally substituted CH, optionally substituted cycloalkyl, optionally substituted heterocycloalkyl, optionally substituted aryl, or optionally substituted heteroaryl;
   TL₁ is of Formula (IB):

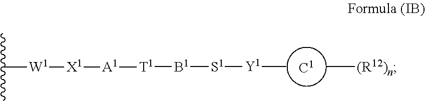

wherein:
W¹ is absent, —O—, —S—, —S(═O)—, —S(═O)₂—, —NR¹¹—, —C(═O)—, —C(═O)NR¹¹—, —NR¹¹C(═O)—, —S(═O)₂NR¹¹—, or —NR¹¹S(═O)₂;
X¹ is C₁-C₁₀alkylene optionally substituted with one or more R and wherein one, two, three, or four carbon atoms of the C₁-C₁₀alkylene are optionally replaced with —S—, —S(═O)—, —S(═O)₂—, —O—, or —NR¹¹—;
A¹ is absent, cycloalkyl, heterocycloalkyl, aryl, or heteroaryl, wherein the cycloalkyl, heterocycloalkyl, aryl, and heteroaryl is optionally substitued with one or more R;
T¹ is absent or C₁-C₃alkylene optionally substituted with one or more R and wherein one carbon atom of the C₁-C₃alkylene is optionally replaced with —C(═O)—, —S—, —S(═O)—, —S(═O)₂—, —O—, —NR¹¹—;
B¹ is cycloalkyl, heterocycloalkyl, or aryl, wherein the cycloalkyl, heterocycloalkyl, and aryl is optionally substitued with one or more R; or
B¹ is heteroaryl substituted with one or more R;
S¹ is absent or C₁-C₃alkylene optionally substituted with one or more R and wherein one or two carbon atoms of the C₁-C₃alkylene are optionally replaced with —C(═O)—, —S—, —S(═O)—, —S(═O)₂—, —O—, or —NR¹¹—;
Y¹ is absent, —O—, —S—, —S(═O)—, —S(═O)₂—, —NR¹¹—, —C(═O)—, —C(═O)NR¹¹—, —NR¹¹C(═O)—, —S(═O)₂NR¹¹—, or —NR¹¹S(═O)₂;
R¹¹ is hydrogen, C₁-C₆alkyl, C₁-C₆haloalkyl, C₁-C₆hydroxyalkyl, C₁-C₆aminoalkyl, or C₁-C₆heteroalkyl;
Ring C¹ is cycloalkyl, heterocycloalkyl, aryl, or heteroaryl;
each R¹² is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
n is 0, 1, 2, 3, 4, 5, or 6;
   TL₂ is of Formula (IIB):

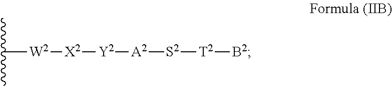

wherein:
W² is absent, —O—, —S—, —S(═O)—, —S(═O)₂—, —NR²¹—, —C(═O)—, —C(═O)NR²¹—, —NR²¹C(═O)—, —S(═O)₂NR²¹—, or —NR²¹S(═O)₂;
X² is absent or C₁-C₁₀alkylene optionally substituted with one or more R, and wherein one, two, three, or four carbon atoms of the C₁-C₁₀alkylene are optionally replaced with —S—, —S(═O)—, —S(═O)₂—, —O—, or —NR²¹—;
Y² is absent, —O—, —S—, —S(═O)—, —S(═O)₂—, —NR²¹—, —C(═O)—, —C(═O)NR²¹—, —NR²¹C(═O)—, —S(═O)₂NR²¹—, or —NR²¹S(═O)₂;
A² is absent, C₁-C₁₀alkylene, cycloalkyl, heterocycloalkyl, aryl, or heteroaryl, wherein the alkylene, cycloalkyl, heterocycloalkyl, aryl, and heteroaryl is optionally substitued with one or more R²²;
each R²² is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
S² is absent, —O—, —S—, —S(═O)—, —S(═O)₂—, —NR²¹—, —C(═O)—, —C(═O)NR²¹—, —NR²¹C(═O)—, —S(═O)₂NR²¹—, or —NR²¹S(═O)₂;
T² is absent or C₁-C₁₀alkylene optionally substituted with one or more R, and wherein one, two, three, or four carbon atoms of the C₁-C₁₀alkylene are optionally replaced with —S—, —S(═O)—, —S(═O)₂—, —O—, or —NR²¹—;
R²¹ is hydrogen, C₁-C₆alkyl, C₁-C₆haloalkyl, C₁-C₆hydroxyalkyl, C₁-C₆aminoalkyl, or C₁-C₆heteroalkyl;
(i) B² is absent;
or
(ii) B² is —N(R²³)₂;
each R²³ is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;

wherein each alkyl, alkenyl, alkynyl, cycloalkyl, heterocycloalkyl, aryl, and heteroaryl is optionally and independently substituted with one or more R;
or
(iii) B² is —CF₃;
or
(iv) B² is cycloalkyl, heterocycloalkyl, aryl, or heteroaryl; wherein the cycloalkyl, heterocycloalkyl, aryl, and heteroaryl is optionally substitued with one or more R²⁴;
each R²⁴ is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
or
(v) B² is

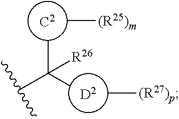

Ring C² is cycloalkyl, heterocycloalkyl, aryl, or heteroaryl;
each R²⁵ is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
m is 0, 1, 2, 3, or 4;
R²⁶ is hydrogen, C₁-C₆alkyl, C₁-C₆haloalkyl, C₁-C₆hydroxyalkyl, C₁-C₆aminoalkyl, or C₁-C₆heteroalkyl;
Ring D² is cycloalkyl, heterocycloalkyl, aryl, or heteroaryl;
each R²⁷ is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
p is 0, 1, 2, 3, or 4;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, -L-cycloalkyl, -L-heterocycloalkyl, -L-aryl, or -L-heteroaryl, wherein each alkyl, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, -L-cycloalkyl, -L-heterocycloalkyl, -L-aryl, or -L-heteroaryl, wherein each alkyl, alkenyl, alkynyl, cycloalkyl, heterocycloalkyl, aryl, and heteroaryl is independently optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, -L-cycloalkyl, -L-heterocycloalkyl, -L-aryl, or -L-heteroaryl, wherein each alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
L is absent or C₁-C₃alkylene optionally substituted with one or more R; and
each R is independently halogen, —CN, —OH, —SF₅, —SH, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, C₃-C₃cycloalkyl, or 3- to 6-membered heterocycloalkyl;
or two R on the same atom form an oxo;
or two R on the same or different atoms are taken together to form a cycloalkyl, heterocycloalkyl, aryl, or heteroaryl.

Also disclosed herein is a conjugate of Formula (B), or a pharmaceutically acceptable salt or stereoisomer thereof:

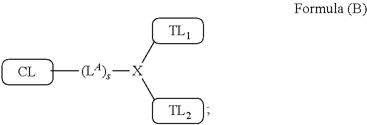

wherein:
   L^{A} represents a linker;
   s is 0 or 1;
   CL represents a metal chelator;
   X is N, optionally substituted CH, optionally substituted cycloalkyl, optionally substituted heterocycloalkyl, optionally substituted aryl, or optionally substituted heteroaryl;
   TL₁ is of Formula (IB):

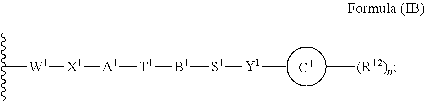

wherein:
W¹ is absent, —C(═O)—, or —C(═O)NR¹¹—;
X¹ is C₁-C₁₀alkylene;
A¹ is absent or heteroaryl optionally substitued with one or more R;
T¹ is absent;
B¹ is cycloalkyl;
R¹¹ is hydrogen;
Ring C¹ is heteroaryl;
each R¹² is independently halogen, —S(═O)₂NR^{c}R^{d}, or —NR^{c}R^{d}; and
n is 0, 1, 2, or 3;
   TL₂ is of Formula (IIB):

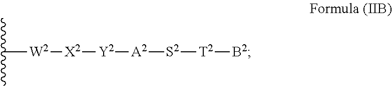

wherein:
W² is absent, —C(═O)—, or —NR²¹C(═O)—;
X² is C₁-C₁₀alkylene optionally substituted with one or more R;
Y² is absent, —NR²¹—, or —NR²¹C(═O)—;
A² is C₁-C₁₀alkylene, cycloalkyl, heterocycloalkyl, aryl, or heteroaryl, wherein the alkylene, cycloalkyl, heterocycloalkyl, aryl, and heteroaryl is optionally substitued with one or more R²²;
each R²² is independently halogen, —CN, —C(═O)OR^{b}, —C(═O)NR^{c}R^{d}, C₁-C₆alkyl, C₁-C₆haloalkyl, or C₁-C₆aminoalkyl;
S² is absent, —NR²¹—, —C(═O)—, or —NR²¹C(═O)—;
T² is absent or C₁-C₁₀alkylene;
R²¹ is hydrogen;
B² is cycloalkyl, heterocycloalkyl, aryl, or heteroaryl; wherein the cycloalkyl, heterocycloalkyl, aryl, and heteroaryl is optionally substitued with one or more R²⁴;
   each R²⁴ is independently halogen, —OH, —SF₅, C₁-C₆alkyl, C₁-C₆haloalkyl, cycloalkyl, or heterocycloalkyl;
or B² is

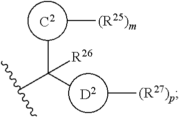

Ring C² is aryl;
each R²⁵ is independently halogen, —OH, or C₁-C₆alkyl;
m is 0, 1, or 2;
R²⁶ is hydrogen or C₁-C₆alkyl;
Ring D² is aryl;
each R²⁷ is independently halogen, —OH, or C₁-C₆alkyl;
p is 0, 1, or 2;
each R^{b} is independently hydrogen or C₁-C₆alkyl;
R^{c} and R^{d} are each independently hydrogen, C₁-C₆alkyl, -L-cycloalkyl, or -L-aryl, wherein each alkyl, cycloalkyl, and aryl is independently optionally substituted with one or more R;
L is absent or C₁-C₃alkylene; and
each R is independently C₁-C₃alkyl, C₁-C₃alkoxy, —C(═O)OH, or —C(═O)NH₂.

Disclosed herein is a conjugate of Formula (BC), or a pharmaceutically acceptable salt or stereoisomer thereof:

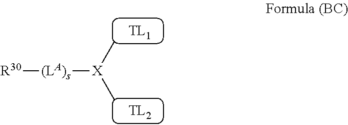

wherein:
   R³⁰ is hydrogen, halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted (e.g., with one or more R); or
   R³⁰ is an albumin binding moiety or a payload moiety;
   wherein the remaining groups have their meanings as defined in Formula (B).

In some embodiments, the conjugate compound of Formula (BC) comprises a covalently bound radioisotope.

Also disclosed herein is a conjugate of Formula (B′), or a pharmaceutically acceptable salt or stereoisomer thereof:

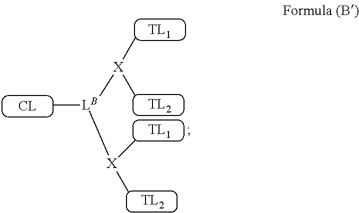

wherein:
   L^{B} represents a linker;
   CL represents a metal chelator;
   each X is independently N, optionally substituted CH, optionally substituted cycloalkyl, optionally substituted heterocycloalkyl, optionally substituted aryl, or optionally substituted heteroaryl;
   each TL₁ represents a 1^{st} targeting moiety that binds to carbonic anhydrase IX; and
   each TL₂ represents a 2^{nd} targeting moiety that binds to carbonic anhydrase IX.

Also disclosed herein is a conjugate of Formula (BC′), or a pharmaceutically acceptable salt or stereoisomer thereof:

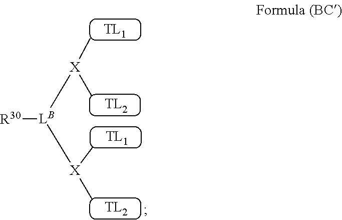

wherein:
   L^{B} represents a linker;
   each X is independently N, optionally substituted CH, optionally substituted cycloalkyl, optionally substituted heterocycloalkyl, optionally substituted aryl, or optionally substituted heteroaryl;
   each TL₁ represents a 1^{st} targeting moiety that binds to carbonic anhydrase IX;
   each TL₂ represents a 2^{nd} targeting moiety that binds to carbonic anhydrase IX;
   R³⁰ is hydrogen, halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted (e.g., with one or more R); or
   R³⁰ is an albumin binding moiety or a payload moiety;
   wherein the remaining groups have their meanings as defined in Formula (B).

In some embodiments, the conjugate compound of Formula (BC′) comprises a covalently bound radioisotope.

In some embodiments of a compound of Formula (B′) or (BC′), each TL₁ is the same. In some embodiments of a compound of Formula (B′) or (BC′), each TL₁ is different. In some embodiments of a compound of Formula (B′) or (BC′), each TL₂ is the same. In some embodiments of a compound of Formula (B′) or (BC′), each TL₂ is different. In some embodiments of a compound of Formula (B′) or (BC′), each X is the same. In some embodiments of a compound of Formula (B′) or (BC′), each X is different. In some embodiments of a compound of Formula (B′) or (BC′), each TL₁ is independently a targeting moiety of Formula (IB). In some embodiments of a compound of Formula (B′) or (BC′), each TL₂ is independently a targeting moiety of Formula (IIB).

In some embodiments of a compound of Formula (B′) or (BC′), each TL₁ is defined in Formula (B). In some embodiments of a compound of Formula (B′) or (BC′), each TL₂ is defined in Formula (B).

In some embodiments of a compound of Formula (B), (BC), (B′), (BC′) or (IB), T¹ is absent or C₁-C₃alkylene.

In some embodiments of a compound of Formula (B), (B′), (BC), (BC′), or (IB), T¹ is absent.

In some embodiments of a compound of Formula (B), (B′), (BC), (BC′), or (IB), B¹ is cycloalkyl optionally substitued with one or more R.

In some embodiments of a compound of Formula (B), (B′), (BC), (BC′), or (IB), B¹ is a monocyclic cycloalkyl optionally substitued with one or more R.

In some embodiments of a compound of Formula (B), (B′), (BC), (BC′), or (IB), B¹ is a bicyclic cycloalkyl optionally substitued with one or more R.

In some embodiments of a compound of Formula (B), (B′), (BC), (BC′), or (IB), B¹ is bicyclo[1.1.1]pentanyl, bicyclo[2.2.1]heptanyl, bicyclo[2.2.2]octanyl, or bicyclo[2.1.1]hexanyl.

In some embodiments of a compound of Formula (B), (B′), (BC), (BC′), or (IB), B¹ is bicyclo[1.1.1]pentanyl.

In some embodiments of a compound of Formula (B), (B′), (BC), (BC′), or (IB), B¹ is heterocycloalkyl, which is optionally substitued with one or more R. In some embodiments of a compound of Formula (B), (B′), (BC), (BC′), or (IB), B¹ is aryl optionally substitued with one or more R. In some embodiments of a compound of Formula (B), (B′), (BC), (BC′), or (IB), B¹ is heteroaryl substituted with one or more R.

In some embodiments of a compound of Formula (B), (B′), (BC), (BC′), or (IB), S¹ is absent.

In some embodiments of a compound of Formula (B), (B′), (BC), (BC′), or (IB),

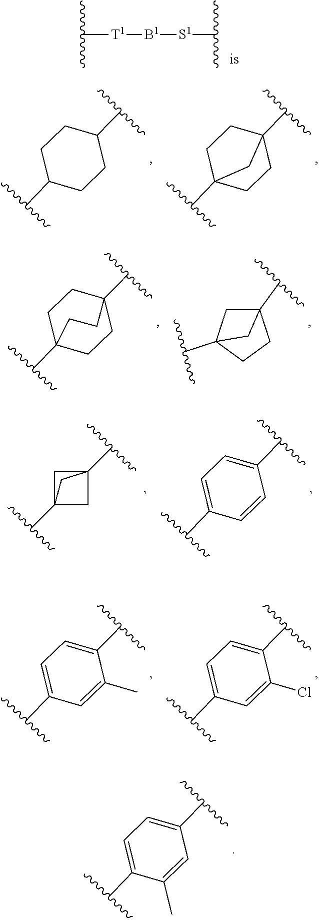

In some embodiments of a compound of Formula (B), (B′), (BC), (BC′), or (IB),

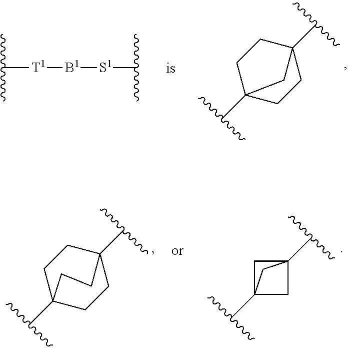

In some embodiments of a compound of Formula (B), (B′), (BC), (BC′), or (IB),

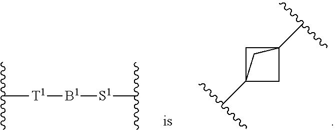

In some embodiments of a compound of Formula (B), (B′), (BC), (BC′), or (IIB), A² is C₁-C₁₀alkylene, heterocycloalkyl, aryl, or heteroaryl, wherein the alkylene, heterocycloalkyl, aryl, and heteroaryl is optionally substitued with one or more R²².

In some embodiments of a compound of Formula (B), (B′), (BC), (BC′), or (IIB), A² is C₁-C₆alkylene optionally substitued with one or more R²².

In some embodiments of a compound of Formula (B), (B′), (BC), (BC′), or (IIB), A² is —CH₂CH[C(═O)OH]—.

In some embodiments of a compound of Formula (B), (B′), (BC), (BC′), or (IIB), A² is heterocycloalkyl optionally substitued with one or more R²².

In some embodiments of a compound of Formula (B), (B′), (BC), (BC′), or (IIB), A² is pyrrolidinyl, piperidinyl, piperazinyl, or morpholinyl, each optionally substitued with one or more R²².

In some embodiments of a compound of Formula (B), (B′), (BC), (BC′), or (IIB), A² is piperidinyl optionally substitued with one or more R²².

In some embodiments of a compound of Formula (B), (B′), (BC), (BC′), or (IIB), A² is

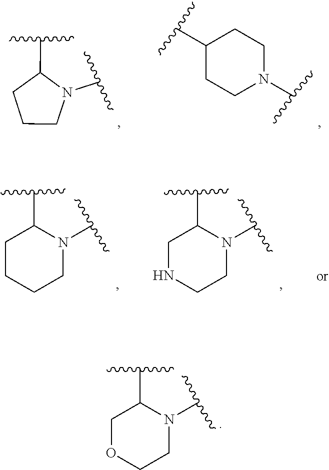 [SMILES: *C1CCCN1* ; *C1CCN(*)CC1 ; *C1CCCCN1* ; *C1CNCCN1* ; *C1COCCN1*]

each optionally substitued with one or more R²².

In some embodiments of a compound of Formula (B), (B′), (BC), (BC′), or (IIB), A² is

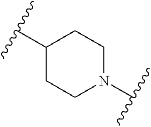 [SMILES: *C1CCN(*)CC1]

optionally substitued with one or more R²².

In some embodiments of a compound of Formula (B), (B′), (BC), (BC′), or (IIB), A² is aryl or heteroaryl, wherein the aryl and heteroaryl is optionally substitued with one or more R²².

In some embodiments of a compound of Formula (B), (B′), (BC), (BC′), or (IIB), A² is phenyl, pyridinyl, pyrimidinyl, or pyrazinyl, each optionally substitued with one or more R²².

In some embodiments of a compound of Formula (B), (B′), (BC), (BC′), or (IIB), A² is

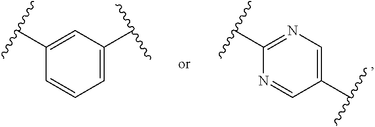 [SMILES: *c1cccc(*)c1 ; *c1cnc(*)nc1]

each optionally substitued with one or more R²².

In some embodiments of a compound of Formula (B), (B′), (BC), (BC′), or (IIB), A² is

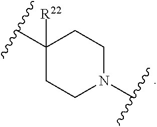

In some embodiments of a compound of Formula (B), (B′), (BC), (BC′), or (IIB), each R²² is independently halogen, —CN, —OH, —OR^{a}, —NR^{c}R^{d}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl.

In some embodiments of a compound of Formula (B), (B′), (BC), (BC′), or (IIB), each R²² is independently —CN, —C(═O)OR^{b}, —C(═O)NR^{c}R^{d}, C₁-C₆alkyl, or C₁-C₆aminoalkyl.

In some embodiments of a compound of Formula (B), (B′), (BC), (BC′), or (IIB), each R²² is independently —C(═O)OR^{b} or —C(═O)NR^{c}R^{d}.

In some embodiments of a compound of Formula (B), (B′), (BC), (BC′), or (IIB), each R²² is independently —C(═O)NR^{c}R^{d}.

In some embodiments of a compound of Formula (B), (B′), (BC), (BC′), or (IIB), A² is

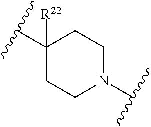

and R²² is halogen, —CN, —OH, —OR^{a}, —NR^{c}R^{d}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl.

In some embodiments of a compound of Formula (B), (B′), (BC), (BC′), or (IIB), A² is

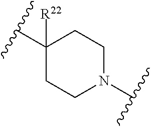

and R²² is —CN, —C(═O)OR^{b}, —C(═O)NR^{c}R^{d}, C₁-C₆alkyl, or C₁-C₆aminoalkyl.

In some embodiments of a compound of Formula (B), (B′), (BC), (BC′), or (IIB), A² is

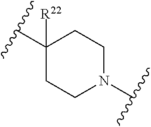

and R²² is —C(═O)OR^{b} or —C(═O)NR^{c}R^{d}.

In some embodiments of a compound of Formula (B), (B′), (BC), (BC′), or (IIB), A² is

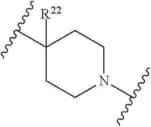

and R²² is —C(═O)NR^{c}R^{d}.

In some embodiments of a compound of Formula (B), (B′), (BC), (BC′), or (IIB), A² is

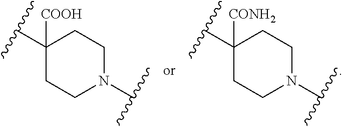 [SMILES: *N1CCC(*)(C(=O)O)CC1 ; *N1CCC(*)(C(N)=O)CC1]

In some embodiments of a compound of Formula (B), (B′), (BC), (BC′), or (IIB), A² is

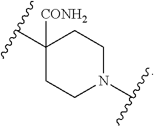 [SMILES: *N1CCC(*)(C(N)=O)CC1]

In some embodiments of a compound of Formula (B), (B′), (BC), (BC′), or (IIB), each R^{a} is independently C₁-C₆alkyl.

In some embodiments of a compound of Formula (B), (B′), (BC), (BC′), or (IIB), each R^{b} is independently hydrogen or C₁-C₆alkyl.

In some embodiments of a compound of Formula (B), (B′), (BC), (BC′), or (IIB), R^{c} and R^{d} are each independently hydrogen, C₁-C₆alkyl, -L-cycloalkyl, or -L-aryl, wherein each alkyl, cycloalkyl, and aryl is independently optionally substituted with one or more R.

In some embodiments of a compound of Formula (B), (B′), (BC), (BC′), or (IIB), L is absent or C₁-C₆alkylene.

In some embodiments of a compound of Formula (B), (B′), (BC), (BC′), or (IIB), each R is independently C₁-C₃alkyl, C₁-C₃alkoxy, —C(═O)OH, or —C(═O)NH₂.

In some embodiments of a compound of Formula (B), (B′), (BC), (BC′), (IIB), (C), (C′), (CC), (CC′), or (IC), TL₁ has a structure of

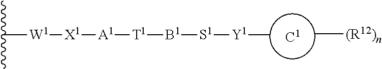

wherein

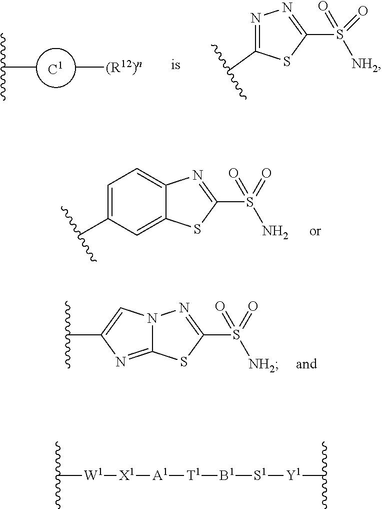

are taken together to form a linking group that comprises 3 to 30 atoms (optionally, 5-25 atoms, 8-20 atoms, 8-15 atoms, or 10-14 atoms, or 8, 9, 10, 11, 12, 13, 14, or 15 atoms) in the linear chain. In some embodiments,

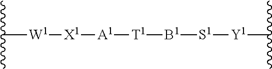

are taken together to form a linking group that comprises 3 to 30 atoms in the linear chain, which are selected from C, N, and O. In some embodiments, the linking group comprises 3 to 30 atoms in the linear chain, including carbon, 1-5 N and 0-1 O. In some embodiments, the linking group comprises 3 to 30 atoms in the linear chain selected from N and C, including 2, 3, or 4 N. In some embodiments, the linking group comprises one or more cyclic groups, and the atoms in the linear chain comprises a part of the cyclic groups. In some embodiments, both A¹ and B¹ are cyclic groups. In some embodiments, the linking group comprises one or more groups selected from —O—, —S—, —S(═O)—, —S(═O)₂—, —NR²¹—, —C(═O)—, —C(═O)NR²¹—, —NR²¹C(═O)—, —S(═O)₂NR²¹—, or —NR²¹S(═O)₂, optionally substituted C₁-C₁₀alkylene, optionally substituted C₁-C₁₀heteroalkylene, optionally substituted cycloalkyl, optionally substituted heterocycloalkyl, optionally substituted aryl, or optionally substituted heteroaryl, or a combination thereof.

In some embodiments of a compound of Formula (B), (B′), (BC), (BC′), (IIB), (C), (C′), (CC), (CC′), or (IC), TL₂ is of the structure of

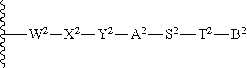

wherein
B² is

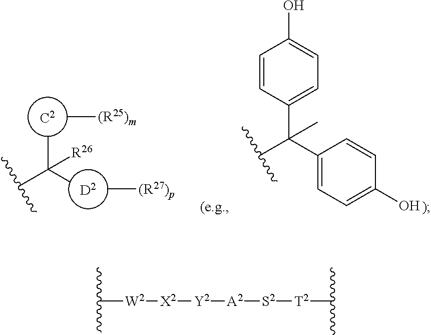

are taken together to form a linking group that comprises 3 to 30 atoms (optionally, 5-25 atoms, 8-20 atoms, 8-15 atoms, or 10-14 atoms, or 8, 9, 10, 11, 12, 13, 14, or 15 atoms) in the linear chain. In some embodiments,

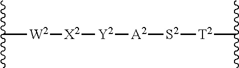

together form a linking group that comprises 3 to 30 atoms in the linear chain, which are selected from C, N, and O. In some embodiments, the linking group comprises 3 to 30 atoms in the linear chain, including carbon, 1-5 N and 0-1 O. In some embodiments, the linking group comprises 3 to 30 atoms in the linear chain selected from N and C, including 2, 3, or 4 N. In some embodiments, the linking group comprises one or more cyclic groups, and the atoms in the linear chain comprises a part of the cyclic groups. In some embodiments, A² is a cyclic group. In some embodiments, the linking group comprises one or more groups selected from —O—, —S—, —S(═O)—, —S(═O)₂—, —NR²¹—, —C(═O)—, —C(═O)NR²¹—, —NR²¹C(═O)—, —S(═O)₂NR²¹—, or —NR²¹S(═O)₂, optionally substituted C₁-C₁₀alkylene, optionally substituted C₁-C₁₀heteroalkylene, optionally substituted cycloalkyl, optionally substituted heterocycloalkyl, optionally substituted aryl, or optionally substituted heteroaryl, or a combination thereof.

Also disclosed herein is a conjugate of Formula (C), or a pharmaceutically acceptable salt or stereoisomer thereof:

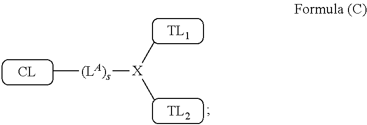

wherein:
   L^{A} represents a linker;
   s is 0 or 1;
   CL represents a metal chelator;
   X is N, optionally substituted CH, optionally substituted cycloalkyl, optionally substituted heterocycloalkyl, optionally substituted aryl, or optionally substituted heteroaryl;
   TL₁ is of Formula (IC):

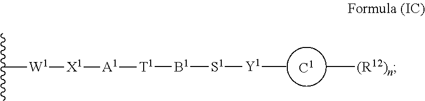

wherein:
W¹ is absent, —O—, —S—, —S(═O)—, —S(═O)₂—, —NR¹¹—, —C(═O)—, —C(═O)NR¹¹—, —NR¹¹C(═O)—, —S(═O)₂NR¹¹—, or —NR¹¹S(═O)₂;
X¹ is C₁-C₁₀alkylene optionally substituted with one or more R and wherein one, two, three, or four carbon atoms of the C₁-C₁₀alkylene are optionally replaced with —S—, —S(═O)—, —S(═O)₂—, —O—, or —NR¹¹—;
A¹ is absent, cycloalkyl, heterocycloalkyl, aryl, or heteroaryl, wherein the cycloalkyl, heterocycloalkyl, aryl, and heteroaryl is optionally substitued with one or more R;
T¹ is absent or C₁-C₃alkylene optionally substituted with one or more R and wherein one carbon atom of the C₁-C₃alkylene is optionally replaced with —C(═O)—, —S—, —S(═O)—, —S(═O)₂—, —O—, —NR¹¹—;
B¹ is absent, C₁-C₁₀alkylene, cycloalkyl, heterocycloalkyl, aryl, or heteroaryl, wherein the alkylene, cycloalkyl, heterocycloalkyl, aryl, and heteroaryl is optionally substitued with one or more R, and wherein one, two, three, or four carbon atoms in the C₁-C₁₀alkylene are optionally replaced with —S—, —S(═O)—, —S(═O)₂—, —O—, —NR¹¹—;
S¹ is absent or C₁-C₃alkylene optionally substituted with one or more R and wherein one or two carbon atoms of the C₁-C₃alkylene are optionally replaced with —S—, —S(═O)—, —S(═O)₂—, —O—, or —NR¹¹—;
Y¹ is absent, —O—, —S—, —S(═O)—, —S(═O)₂—, —NR¹¹—, —C(═O)—, —C(═O)NR¹¹—, —NR¹¹C(═O)—, —S(═O)₂NR¹¹—, or —NR¹¹S(═O)₂;
R¹¹ is hydrogen, C₁-C₆alkyl, C₁-C₆haloalkyl, C₁-C₆hydroxyalkyl, C₁-C₆aminoalkyl, or C₁-C₆heteroalkyl;
Ring C¹ is cycloalkyl, heterocycloalkyl, aryl, or heteroaryl;
each R¹² is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
n is 0, 1, 2, 3, 4, 5, or 6;
   TL₂ is of Formula (IIC):

—W²—X²—Y²-A²-S²-T²-B² Formula (IIC);
wherein:
W² is absent, —O—, —S—, —S(═O)—, —S(═O)₂—, —NR²¹—, —C(═O)—, —C(═O)NR²¹—, —NR²¹C(═O)—, —S(═O)₂NR²¹—, or —NR²¹S(═O)₂;
X² is absent or C₁-C₁₀alkylene optionally substituted with one or more R, and wherein one, two, three, or four carbon atoms of the C₁-C₁₀alkylene are optionally replaced with —S—, —S(═O)—, —S(═O)₂—, —O—, or —NR²¹—;
Y² is absent, —O—, —S—, —S(═O)—, —S(═O)₂—, —NR²¹—, —C(═O)—, —C(═O)NR²¹—, —NR²¹C(═O)—, —S(═O)₂NR²¹—, or —NR²¹S(═O)₂;
A² is cycloalkyl, heterocycloalkyl, aryl, or heteroaryl, wherein the cycloalkyl, heterocycloalkyl, aryl, and heteroaryl is optionally substitued with one or more R²²;
each R²² is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
S² is absent, —O—, —S—, —S(═O)—, —S(═O)₂—, —NR²¹—, —C(═O)—, —C(═O)NR²¹—, —NR²¹C(═O)—, —S(═O)₂NR²¹—, or —NR²¹S(═O)₂;
T² is absent or C₁-C₁₀alkylene optionally substituted with one or more R, and wherein one, two, three, or four carbon atoms of the C₁-C₁₀alkylene are optionally replaced with —S—, —S(═O)—, —S(═O)₂—, —O—, or —NR²¹—;
R²¹ is hydrogen, C₁-C₆alkyl, C₁-C₆haloalkyl, C₁-C₆hydroxyalkyl, C₁-C₆aminoalkyl, or C₁-C₆heteroalkyl;
(i) B² is absent;
or
(ii) B² is —N(R²³)₂;
each R²³ is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
or
(iii) B² is —CF₃;
or
(iv) B² is cycloalkyl, heterocycloalkyl, aryl, or heteroaryl; wherein the cycloalkyl, heterocycloalkyl, aryl, and heteroaryl is optionally substitued with one or more R²⁴;
each R²⁴ is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
or
(v) B² is

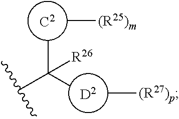

Ring C² is cycloalkyl, heterocycloalkyl, aryl, or heteroaryl;
each R²⁵ is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
m is 0, 1, 2, 3, or 4;
R²⁶ is hydrogen, C₁-C₆alkyl, C₁-C₆haloalkyl, C₁-C₆hydroxyalkyl, C₁-C₆aminoalkyl, or C₁-C₆heteroalkyl;
Ring D² is cycloalkyl, heterocycloalkyl, aryl, or heteroaryl;
each R²⁷ is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
p is 0, 1, 2, 3, or 4;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, -L-cycloalkyl, -L-heterocycloalkyl, -L-aryl, or -L-heteroaryl, wherein each alkyl, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, -L-cycloalkyl, -L-heterocycloalkyl, -L-aryl, or -L-heteroaryl, wherein each alkyl, alkenyl, alkynyl, cycloalkyl, heterocycloalkyl, aryl, and heteroaryl is independently optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, -L-cycloalkyl, -L-heterocycloalkyl, -L-aryl, or -L-heteroaryl, wherein each alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
L is absent or C₁-C₃alkylene optionally substituted with one or more R; and
each R is independently halogen, —CN, —OH, —SF₅, —SH, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, C₃-C₆cycloalkyl, or 3- to 6-membered heterocycloalkyl;
or two R on the same atom form an oxo;

or two R on the same or different atoms are taken together to form a cycloalkyl, heterocycloalkyl, aryl, or heteroaryl.

Also disclosed herein is a conjugate of Formula (C), or a pharmaceutically acceptable salt or stereoisomer thereof:

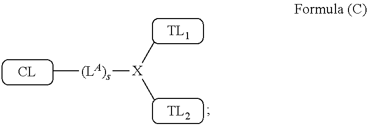

wherein:
   L^{A} represents a linker;
   s is 0 or 1;
   CL represents a metal chelator;
   X is N, optionally substituted CH, optionally substituted cycloalkyl, optionally substituted heterocycloalkyl, optionally substituted aryl, or optionally substituted heteroaryl;
   TL₁ is of Formula (IC):

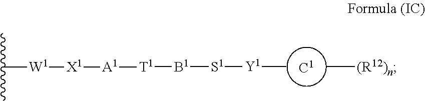

wherein:
W¹ is absent, —C(═O)—, or —C(═O)NR¹¹—;
X¹ is C₁-C₁₀alkylene;
A¹ is absent or heteroaryl optionally substitued with one or more R;
T¹ is absent;
B¹ is C₁-C₁₀alkylene or cycloalkyl;
S¹ is absent;
Y¹ is absent, —O—, —S—, —S(═O)₂—, —C(═O)NR¹¹—, or —NR¹¹C(═)—;
R¹¹ is hydrogen;
Ring C¹ is heteroaryl;
each R¹² is independently halogen, —S(═O)₂NR^{c}R^{d}, or —NR^{c}R^{d}; and
n is 0, 1, 2, or 3;
   TL₂ is of Formula (IIC):

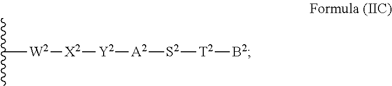

wherein:
W² is absent, —C(═O)—, or —NR²¹C(═O)—;
X² is C₁-C₁₀alkylene optionally substituted with one or more R;
Y² is absent, —NR²¹—, or —NR²¹C(═O)—;
A² is cycloalkyl, heterocycloalkyl, aryl, or heteroaryl, wherein the alkylene, cycloalkyl, heterocycloalkyl, aryl, and heteroaryl is optionally substitued with one or more R²²;
each R²² is independently halogen, —CN, —C(═O)OR^{b}, —C(═O)NR^{c}R^{d}, C₁-C₆alkyl, C₁-C₆haloalkyl, or C₁-C₆aminoalkyl;
S² is absent, —NR²¹—, —C(═O)—, or —NR²¹C(═O)—;
T² is absent or C₁-C₁₀alkylene;
R²¹ is hydrogen;
B² is cycloalkyl, heterocycloalkyl, aryl, or heteroaryl; wherein the cycloalkyl, heterocycloalkyl, aryl, and heteroaryl is optionally substitued with one or more R²⁴;
   each R²⁴ is independently halogen, —OH, —SF₅, C₁-C₆alkyl, C₁-C₆haloalkyl, cycloalkyl, or heterocycloalkyl;
or B² is

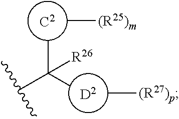

Ring C² is aryl;
each R²⁵ is independently halogen, —OH, or C₁-C₆alkyl;
m is 0, 1, or 2;
R²⁶ is hydrogen or C₁-C₆alkyl;
Ring D² is aryl;
each R²⁷ is independently halogen, —OH, or C₁-C₆alkyl;
p is 0, 1, or 2;
each R^{b} is independently hydrogen or C₁-C₆alkyl;
R^{c} and R^{d} are each independently hydrogen, C₁-C₆alkyl, -L-cycloalkyl, or -L-aryl, wherein each alkyl, cycloalkyl, and aryl is independently optionally substituted with one or more R;
L is absent or C₁-C₃alkylene; and
each R is independently C₁-C₃alkyl, C₁-C₃alkoxy, —C(═O)OH, or —C(═O)NH₂.

Disclosed herein is a conjugate of Formula (CC), or a pharmaceutically acceptable salt or stereoisomer thereof:

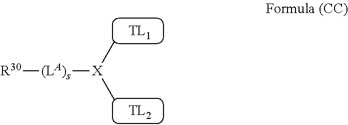

wherein:
   R³⁰ is hydrogen, halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted (e.g., with one or more R); or
   R³⁰ is an albumin binding moiety or a payload moiety;
   wherein the remaining groups have their meanings as defined in Formula (C).

In some embodiments, the conjugate compound of Formula (CC) comprises a covalently bound radioisotope.

Also disclosed herein is a conjugate of Formula (C′), or a pharmaceutically acceptable salt or stereoisomer thereof:

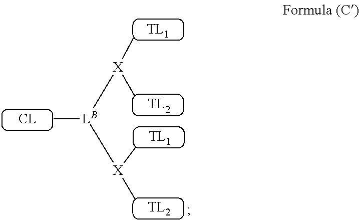

wherein:
   L^{B} represents a linker;
   CL represents a metal chelator;
   each X is independently N, optionally substituted CH, optionally substituted cycloalkyl, optionally substituted heterocycloalkyl, optionally substituted aryl, or optionally substituted heteroaryl;
   each TL₁ represents a 1^{st} targeting moiety that binds to carbonic anhydrase IX; and
each TL₂ represents a 2^{nd} targeting moiety that binds to carbonic anhydrase IX.

Also disclosed herein is a conjugate of Formula (CC′), or a pharmaceutically acceptable salt or stereoisomer thereof:

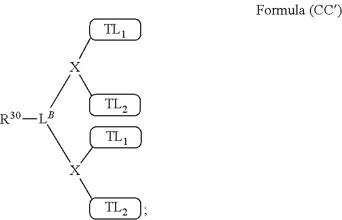

wherein:
   L^{B} represents a linker;
   each X is independently N, optionally substituted CH, optionally substituted cycloalkyl, optionally substituted heterocycloalkyl, optionally substituted aryl, or optionally substituted heteroaryl;
   each TL₁ represents a 1^{st} targeting moiety that binds to carbonic anhydrase IX;
   each TL₂ represents a 2^{nd} targeting moiety that binds to carbonic anhydrase IX; and
   R³⁰ is hydrogen, halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted (e.g., with one or more R); or
   R³⁰ is an albumin binding moiety or a payload moiety.

In some embodiments, the conjugate compound of Formula (CC′) comprises a covalently bound radioisotope.

In some embodiments of a compound of Formula (C′) or (CC′), each TL₁ is the same. In some embodiments of a compound of Formula (C′) or (CC′), each TL₁ is different. In some embodiments of a compound of Formula (C′) or (CC′), each TL₂ is the same. In some embodiments of a compound of Formula (C′) or (CC′), each TL₂ is different. In some embodiments of a compound of Formula (C′) or (CC′), each X is the same. In some embodiments of a compound of Formula (C′) or (CC′), each X is different. In some embodiments of a compound of Formula (C′) or (CC′), each TL₁ is independently a targeting moiety of Formula (IC). In some embodiments of a compound of Formula (C′) or (CC′), each TL₂ is independently a targeting moiety of Formula (IIC).

In some embodiments of a compound of Formula (C′) or (CC′), each TL₁ is defined in Formula (C). In some embodiments of a compound of Formula (C′) or (CC′), each TL₂ is defined in Formula (C).

In some embodiments of a compound of Formula (C), (C′), (CC), (CC′), or (IC), T¹ is absent or C₁-C₃alkylene.

In some embodiments of a compound of Formula (C), (C′), (CC), (CC′), or (IC), T¹ is absent.

In some embodiments of a compound of Formula (C), (C′), (CC), (CC′), or (IC), B¹ is C₁-C₁₀alkylene or cycloalkyl, wherein the alkylene and cycloalkyl is optionally substitued with one or more R, and wherein one, two, three, or four carbon atoms in the C₁-C₁₀alkylene are optionally replaced with —S—, —S(═O)—, —S(═O)₂—, —O—, or —NR¹¹—.

In some embodiments of a compound of Formula (C), (C′), (CC), (CC′), or (IC), B¹ is C₁-C₁₀alkylene optionally substitued with one or more R, and wherein one, two, three, or four carbon atoms in the C₁-C₁₀alkylene are optionally replaced with —S—, —S(═O)—, —S(═O)₂—, —O—, or —NR¹¹—.

In some embodiments of a compound of Formula (C), (C′), (CC), (CC′), or (IC), B¹ is C₁-C₁₀alkylene optionally substitued with one or more R.

In some embodiments of a compound of Formula (C), (C′), (CC), (CC′), or (IC), B¹ is C₁-C₆alkylene.

In some embodiments of a compound of Formula (C), (C′), (CC), (CC′), or (IC), B¹ is —CH₂CH₂CH₂—.

In some embodiments of a compound of Formula (C), (C′), (CC), (CC′), or (IC), B¹ is —CH₂CH₂CH₂CH₂—.

In some embodiments of a compound of Formula (C), (C′), (CC), (CC′), or (IC), B¹ is cycloalkyl optionally substitued with one or more R.

In some embodiments of a compound of Formula (C), (C′), (CC), (CC′), or (IC), B¹ is a monocyclic cycloalkyl optionally substitued with one or more R.

In some embodiments of a compound of Formula (C), (C′), (CC), (CC′), or (IC), B¹ is a bicyclic cycloalkyl optionally substitued with one or more R.

In some embodiments of a compound of Formula (C), (C′), (CC), (CC′), or (IC), B¹ is bicyclo[1.1.1]pentanyl, bicyclo[2.2.1]heptanyl, bicyclo[2.2.2]octanyl, or bicyclo[2.1.1]hexanyl.

In some embodiments of a compound of Formula (C), (C′), (CC), (CC′), or (IC), B¹ is bicyclo[1.1.1]pentanyl.

In some embodiments of a compound of Formula (C), (C′), (CC), (CC′), or (IC), S¹ is absent.

In some embodiments of a compound of Formula (C), (C′), (CC), (CC′), or (IC),

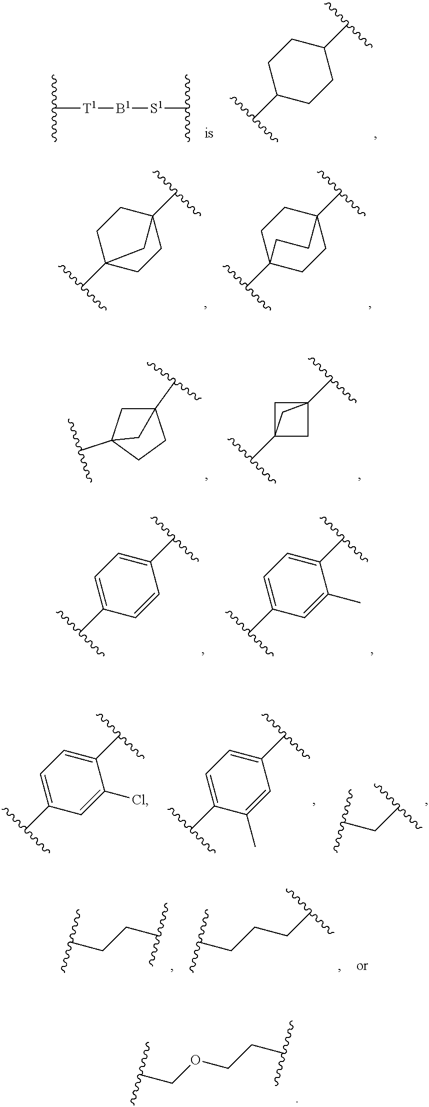

In some embodiments of a compound of Formula (C), (C′), (CC), (CC′), or (IC),

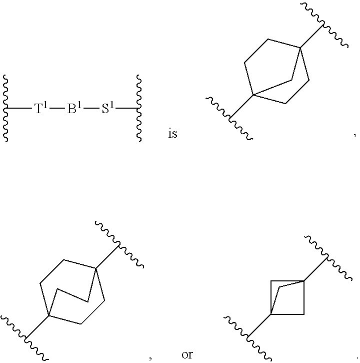

In some embodiments of a compound of Formula (C), (C′), (CC), (CC′), or (IC),

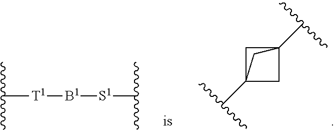

In some embodiments of a compound of Formula (C), (C′), (CC), (CC′), or (IIC), A² is heterocycloalkyl, aryl, or heteroaryl, wherein the heterocycloalkyl, aryl, and heteroaryl is optionally substitued with one or more R²².

In some embodiments of a compound of Formula (C), (C′), (CC), (CC′), or (IIC), A² is heterocycloalkyl optionally substitued with one or more R²².

In some embodiments of a compound of Formula (C), (C′), (CC), (CC′), or (IIC), A² is pyrrolidinyl, piperidinyl, piperazinyl, or morpholinyl, each optionally substitued with one or more R²².

In some embodiments of a compound of Formula (C), (C′), (CC), (CC′), or (IIC), A² is piperidinyl optionally substitued with one or more R²².

In some embodiments of a compound of Formula (C), (C′), (CC), (CC′), or (IIC), A² is

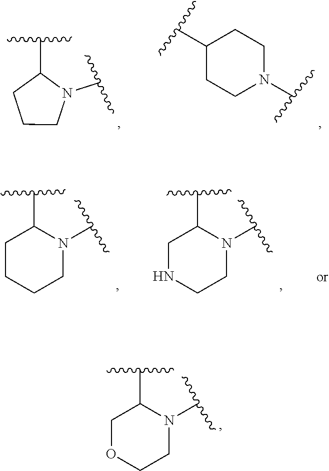 [SMILES: *C1CCCN1* ; *C1CCN(*)CC1 ; *C1CCCCN1* ; *C1CNCCN1* ; *C1COCCN1*]

each optionally substitued with one or more R²².

In some embodiments of a compound of Formula (C), (C′), (CC), (CC′), or (IIC), A² is

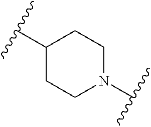 [SMILES: *C1CCN(*)CC1]

optionally substitued with one or more R²².

In some embodiments of a compound of Formula (C), (C′), (CC), (CC′), or (IIC), A² is aryl or heteroaryl, wherein the aryl and heteroaryl is optionally substitued with one or more R²².

In some embodiments of a compound of Formula (C), (C′), (CC), (CC′), or (IIC), A² is phenyl, pyridinyl, pyrimidinyl, or pyrazinyl, each optionally substitued with one or more R²².

In some embodiments of a compound of Formula (C), (C′), (CC), (CC′), or (IIC), A² is

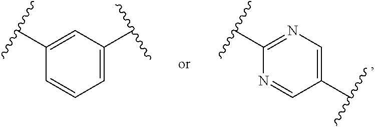 [SMILES: *c1cccc(*)c1 ; *c1cnc(*)nc1]

each optionally substitued with one or more R²².

In some embodiments of a compound of Formula (C), (C′), (CC), (CC′), or (IIC), A² is

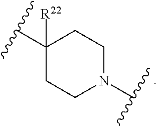

In some embodiments of a compound of Formula (C), (C′), (CC), (CC′), or (IIC), each R²² is independently halogen, —CN, —OH, —OR^{a}, —NR^{c}R^{d}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl.

In some embodiments of a compound of Formula (C), (C′), (CC), (CC′), or (IIC), each R²² is independently —CN, —C(═O)OR^{b}, —C(═O)NR^{c}R^{d}, C₁-C₆alkyl, or C₁-C₆aminoalkyl.

In some embodiments of a compound of Formula (C), (C′), (CC), (CC′), or (IIC), each R²² is independently —C(═O)OR^{b} or —C(═O)NR^{c}R^{d}.

In some embodiments of a compound of Formula (C), (C′), (CC), (CC′), or (IIC), each R²² is independently —C(═O)NR^{c}R^{d}.

In some embodiments of a compound of Formula (C), (C′), (CC), (CC′), or (IIC), A² is

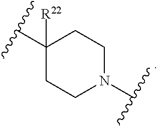

and R²² is halogen, —CN, —OH, —OR^{a}, —NR^{c}R^{d}, —C(═O)OR_{b}, —C(═O)NR^{c}R^{d}, C₁-C₆alkyl, C₁-C₆haloalkyl, C₁-C₆hydroxyalkyl, C₁-C₆aminoalkyl, or C₁-C₆heteroalkyl.

In some embodiments of a compound of Formula (C), (C′), (CC), (CC′), or (IIC), A² is

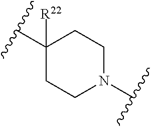

and R²² is —CN, —C(═O)OR^{b}, —C(═O)NR^{c}R^{d}, C₁-C₆alkyl, or C₁-C₆aminoalkyl.

In some embodiments of a compound of Formula (C), (C′), (CC), (CC′), or (IIC), A² is

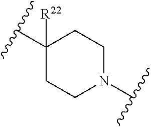

and R²² is —C(═O)OR^{b} or —C(═O)NR^{c}R^{d}.

In some embodiments of a compound of Formula (C), (C′), (CC), (CC′), or (IIC), A² is

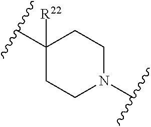

and R²² is —C(═O)NR^{c}R^{d}.

In some embodiments of a compound of Formula (C), (C′), (CC), (CC′), or (IIC), A² is

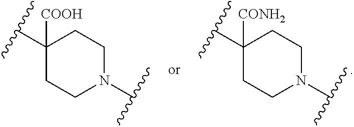 [SMILES: *N1CCC(*)(C(=O)O)CC1 ; *N1CCC(*)(C(N)=O)CC1]

In some embodiments of a compound of Formula (C), (C′), (CC), (CC′), or (IIC), A² is

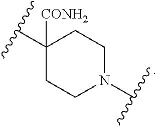 [SMILES: *N1CCC(*)(C(N)=O)CC1]

In some embodiments of a compound of Formula (C), (C′), (CC), (CC′), or (IIC), each R^{a} is independently or C₁-C₆alkyl.

In some embodiments of a compound of Formula (C), (C′), (CC), (CC′), or (IIC), each R^{b} is independently hydrogen or C₁-C₆alkyl.

In some embodiments of a compound of Formula (C), (C′), (CC), (CC′), or (IIC), R^{c} and R^{d} are each independently hydrogen, C₁-C₆alkyl, -L-cycloalkyl, or -L-aryl, wherein each alkyl, cycloalkyl, and aryl is independently optionally substituted with one or more R.

In some embodiments of a compound of Formula (C), (C′), (CC), (CC′), or (IIC), L is absent or C₁-C₃alkylene.

In some embodiments of a compound of Formula (C), (C′), (CC), (CC′), or (IIC), each R is independently C₁-C₃alkyl, C₁-C₃alkoxy, —C(═O)OH, or —C(═O)NH₂.

In some embodiments of a compound of Formula (B), (B′), (BC), (BC′), (C), (C′), (CC), or (CC′), X is CH. In some embodiments of a compound of Formula (B), (B′), (BC), (BC′), (C), (C′), (CC), or (CC′), X is CH optionally substituted with halogen or C₁-C₆alkyl. In some embodiments of a compound of Formula (B), (B′), (BC), (BC′), (C), (C′), (CC), or (CC′), X is N.

In some embodiments of a compound of Formula (B), (B′), (BC), (BC′), (C), (C′), (CC), or (CC′), X is cycloalkyl, heterocycloalkyl, aryl, or heteroaryl, wherein the cycloalkyl, heterocycloalkyl, aryl, and heteroaryl is optionally substituted with one or more R; and
each R is independently halogen, —CN, —OH, —SF₅, —SH, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, C₃-C₆cycloalkyl, or 3- to 6-membered heterocycloalkyl; or two R on the same atom form an oxo.

In some embodiments of a compound of Formula (B), (B′), (BC), (BC′), (C), (C′), (CC), or (CC′), X is optionally substituted heterocycloalkyl.

In some embodiments of a compound of Formula (B), (B′), (BC), (BC′), (C), (C′), (CC), or (CC′), X is heterocycloalkyl optionally substituted with one or more R.

In some embodiments of a compound of Formula (B), (B′), (BC), (BC′), (C), (C′), (CC), or (CC′), X is monocyclic heterocycloalkyl optionally substituted with one or more R.

In some embodiments of a compound of Formula (B), (B′), (BC), (BC′), (C), (C′), (CC), or (CC′), X is bicyclic heterocycloalkyl optionally substituted with one or more R. In some embodiments of a compound of Formula (B), (B′), (BC), (BC′), (C), (C′), (CC), or (CC′), X is spirobicyclic heterocycloalkyl optionally substituted with one or more

In some embodiments of a compound of Formula (B), (B′), (BC), (BC′), (C), (C′), (CC), or (CC′), X is 4- to 6-membered heterocycloalkyl optionally substituted with one or more R. In some embodiments of a compound of Formula (B), (B′), (BC), (BC′), (C), (C′), (CC), or (CC′), X is azetidinyl, pyrrolidinyl, piperidinyl, or piperazinyl, each optionally substituted with one or more R. In some embodiments of a compound of Formula (B), (B′), (BC), (BC′), (C), (C′), (CC), or (CC′), X is piperidinyl or piperazinyl, each optionally substituted with one or more R. In some embodiments of a compound of Formula (B), (B′), (BC), (BC′), (C), (C′), (CC), or (CC′), X is piperidinyl optionally substituted with one or more R. In some embodiments of a compound of Formula (B), (B′), (BC), (BC′), (C), (C′), (CC), or (CC′), X is piperazinyl optionally substituted with one or more R.

In some embodiments of a compound of Formula (B), (B′), (BC), (BC′), (C), (C′), (CC), or (CC′),

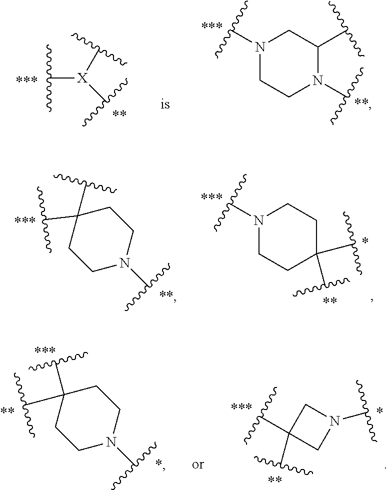

In some embodiments of a compound of Formula (B), (B′), (BC), (BC′), (C), (C′), (CC), or (CC′),

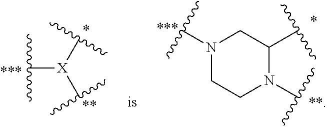

In some embodiments of a compound of Formula (B), (B′), (BC), (BC′), (C), (C′), (CC), or (CC′),

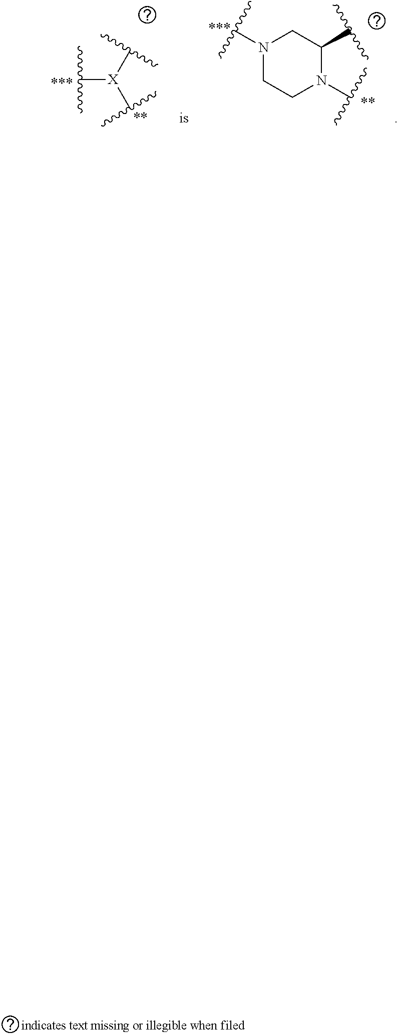

In some embodiments of a compound of Formula (B), (B′), (BC), (BC′), (C), (C′), (CC), or (CC′),

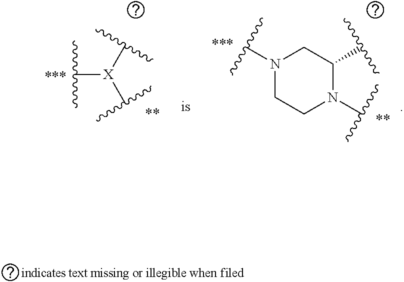

In some embodiments of a compound of Formula (B), (B′), (BC), (BC′), (C), (C′), (CC), or (CC′),

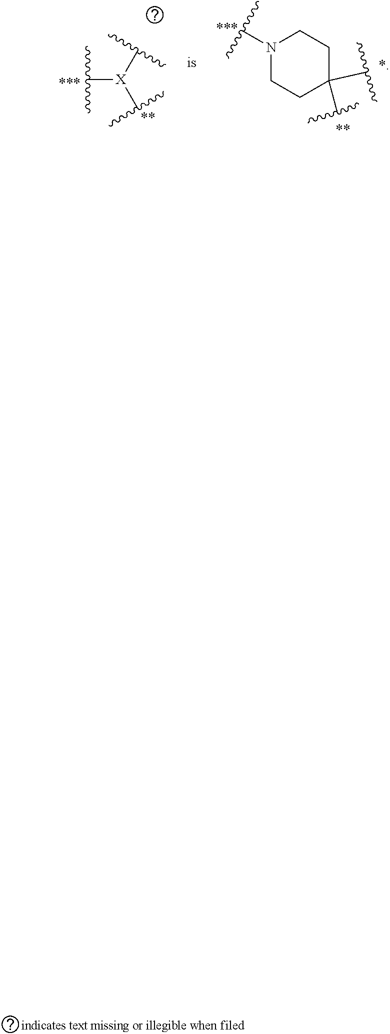

In some embodiments of a compound of Formula (B), (B′), (IB), (C), (C′), (IC), (BC), (BC′), (CC), or (CC′), W¹ is absent, —O—, —S—, —NR¹¹—, —C(═O)—, —C(═O)NR¹¹—, or —NR¹¹C(═O)—.

In some embodiments of a compound of Formula (B), (B′), (IB), (C), (C′), (IC), (BC), (BC′), (CC), or (CC′), W¹ is absent, —C(═O)—, or —C(═O)NR¹¹—.

In some embodiments of a compound of Formula (B), (B′), (IB), (C), (C′), (IC), (BC), (BC′), (CC), or (CC′), W¹ is absent.

In some embodiments of a compound of Formula (B), (B′), (IB), (C), (C′), (IC), (BC), (BC′), (CC), or (CC′), W¹ is —C(═O)—.

In some embodiments of a compound of Formula (B), (B′), (IB), (C), (C′), (IC), (BC), (BC′), (CC), or (CC′), W¹ is —C(═O)NR¹¹—.

In some embodiments of a compound of Formula (B), (B′), (IB), (C), (C′), (IC), (BC), (BC′), (CC), or (CC′), X¹ is C₁-C₆alkylene optionally substituted with one or more R and wherein one or two carbon atoms of the C₁-C₆alkylene are optionally replaced with —S—, —S(═O)—, —S(═O)₂—, —O—, or —NR¹¹—.

In some embodiments of a compound of Formula (B), (B′), (IB), (C), (C′), (IC), (BC), (BC′), (CC), or (CC′), X¹ is C₁-C₆alkylene.

In some embodiments of a compound of Formula (B), (B′), (IB), (C), (C′), (IC), (BC), (BC′), (CC), or (CC′), X¹ is C₁-C₄alkylene.

In some embodiments of a compound of Formula (B), (B′), (IB), (C), (C′), (IC), (BC), (BC′), (CC), or (CC′), X¹ is —CH₂—.

In some embodiments of a compound of Formula (B), (B′), (IB), (C), (C′), (IC), (BC), (BC′), (CC), or (CC′), X¹ is —CH₂CH₂—.

In some embodiments of a compound of Formula (B), (B′), (IB), (C), (C′), (IC), (BC), (BC′), (CC), or (CC′), X¹ is —CH₂CH₂CH₂—.

In some embodiments of a compound of Formula (B), (B′), (IB), (C), (C′), (IC), (BC), (BC′), (CC), or (CC′), X¹ is —CH₂CH₂CH₂CH₂—.

In some embodiments of a compound of Formula (B), (B′), (IB), (C), (C′), (IC), (BC), (BC′), (CC), or (CC′),

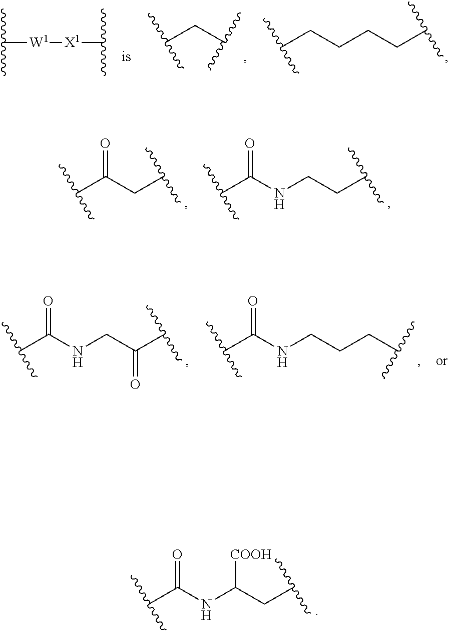

In some embodiments of a compound of Formula (B), (B′), (IB), (C), (C′), (IC), (BC), (BC′), (CC), or (CC′),

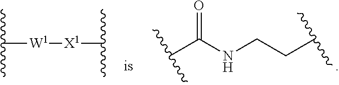

In some embodiments of a compound of Formula (B), (B′), (IB), (C), (C′), (IC), (BC), (BC′), (CC), or (CC′), A¹ is absent.

In some embodiments of a compound of Formula (B), (B′), (IB), (C), (C′), (IC), (BC), (BC′), (CC), or (CC′), A¹ is heteroaryl optionally substitued with one or more R.

In some embodiments of a compound of Formula (B), (B′), (IB), (C), (C′), (IC), (BC), (BC′), (CC), or (CC′), A¹ is triazolyl optionally substitued with one or more R.

In some embodiments of a compound of Formula (B), (B′), (IB), (C), (C′), (IC), (BC), (BC′), (CC), or (CC′), A¹ is

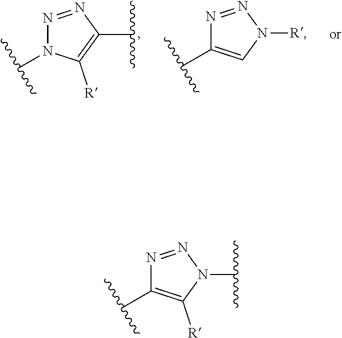

wherein R′ is hydrogen or R. In some embodiments of a compound of Formula (B), (B′), (IB), (C), (C′), (IC), (BC), (BC′), (CC), or (CC′), A¹ is

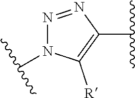

wherein R′ is hydrogen or R. In some embodiments, R′ is hydrogen.

In some embodiments of a compound of Formula (B), (B′), (IB), (C), (C′), (IC), (BC), (BC′), (CC), or (CC′), A¹ is heterocycloalkyl optionally substitued with one or more R.

In some embodiments of a compound of Formula (B), (B′), (IB), (C), (C′), (IC), (BC), (BC′), (CC), or (CC′), A¹ is monocyclic heterocycloalkyl optionally substitued with one or more R.

In some embodiments of a compound of Formula (B), (B′), (IB), (C), (C′), (IC), (BC), (BC′), (CC), or (CC′), A¹ is bicyclic heterocycloalkyl optionally substitued with one or more R.

In some embodiments of a compound of Formula (B), (B′), (IB), (C), (C′), (IC), (BC), (BC′), (CC), or (CC′), A¹ is

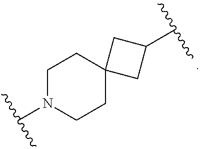 [SMILES: *C1CC2(CCN(*)CC2)C1]

In some embodiments of a compound of Formula (B), (B′), (IB), (C), (C′), (IC), (BC), (BC′), (CC), or (CC′), T¹ is absent or C₁-C₃alkylene.

In some embodiments of a compound of Formula (B), (B′), (IB), (C), (C′), (IC), (BC), (BC′), (CC), or (CC′), T¹ is absent.

In some embodiments of a compound of Formula (B), (B′), (IB), (C), (C′), (IC), (BC), (BC′), (CC), or (CC′), B¹ is cycloalkyl optionally substitued with one or more R.

In some embodiments of a compound of Formula (B), (B′), (IB), (C), (C′), (IC), (BC), (BC′), (CC), or (CC′), B¹ is a monocyclic cycloalkyl optionally substitued with one or more R.

In some embodiments of a compound of Formula (B), (B′), (IB), (C), (C′), (IC), (BC), (BC′), (CC), or (CC′), B¹ is a bicyclic cycloalkyl optionally substitued with one or more R.

In some embodiments of a compound of Formula (B), (B′), (IB), (C), (C′), (IC), (BC), (BC′), (CC), or (CC′), B¹ is bicyclo[1.1.1]pentanyl, bicyclo[2.2.1]heptanyl, bicyclo[2.2.2]octanyl, or bicyclo[2.1.1]hexanyl.

In some embodiments of a compound of Formula (B), (B′), (IB), (C), (C′), (IC), (BC), (BC′), (CC), or (CC′), B¹ is bicyclo[1.1.1]pentanyl.

In some embodiments of a compound of Formula (B), (B′), (IB), (C), (C′), (IC), (BC), (BC′), (CC), or (CC′), S¹ is absent.

In some embodiments of a compound of Formula (B), (B′), (IB), (C), (C′), (IC), (BC), (BC′), (CC), or (CC′),

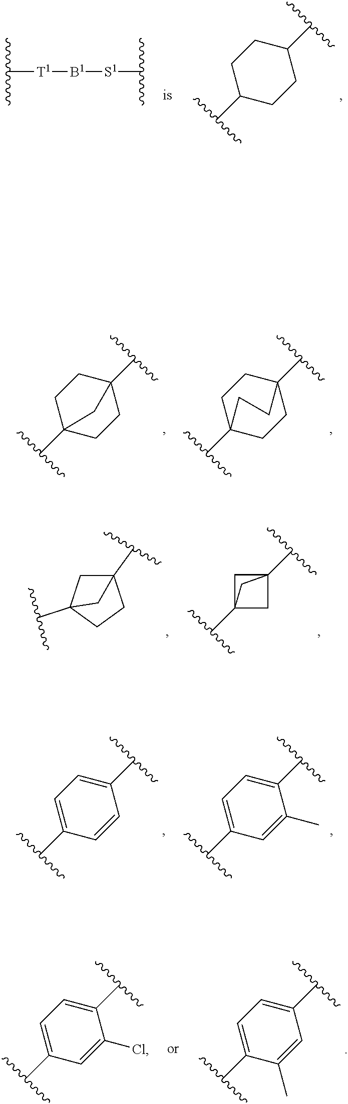

In some embodiments of a compound of Formula (B), (B′), (IB), (C), (C′), (IC), (BC), (BC′), (CC), or (CC′),

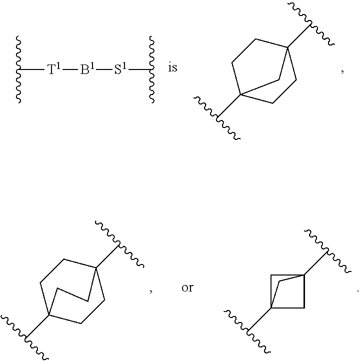

In some embodiments of a compound of Formula (B), (B′), (IB), (C), (C′), (IC), (BC), (BC′), (CC), or (CC′),

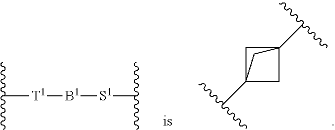

In some embodiments of a compound of Formula (B), (B′), (IB), (C), (C′), (IC), (BC), (BC′), (CC), or (CC′), Y¹ is absent, —O—, —S—, —S(═O)₂—, —C(═O)NR¹¹—, or —NR¹¹C(═O)—.

In some embodiments of a compound of Formula (B), (B′), (IB), (C), (C′), (IC), (BC), (BC′), (CC), or (CC′), Y¹ is —S(═O)₂—.

In some embodiments of a compound of Formula (B), (B′), (IB), (C), (C′), (IC), (BC), (BC′), (CC), or (CC′), Y¹ is —C(═O)NR¹¹—.

In some embodiments of a compound of Formula (B), (B′), (IB), (C), (C′), (IC), (BC), (BC′), (CC), or (CC′),

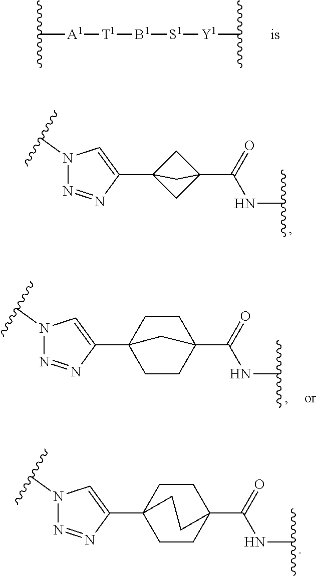

In some embodiments of a compound of Formula (B), (B′), (IB), (C), (C′), (IC), (BC), (BC′), (CC), or (CC′),

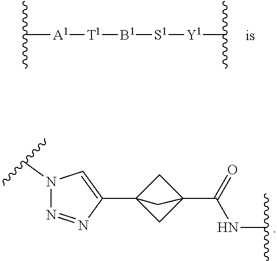

In some embodiments of a compound of Formula (B), (B′), (IB), (C), (C′), (IC), (BC), (BC′), (CC), or (CC′), Ring C¹ is aryl or heteroaryl. In some embodiments of a compound of Formula (B), (B′), (IB), (C), (C′), (IC), (BC), (BC′), (CC), or (CC′), Ring C¹ is phenyl or heteroaryl. In some embodiments of a compound of Formula (B), (B′), (IB), (C), (C′), (IC), (BC), (BC′), (CC), or (CC′), Ring C¹ is heteroaryl. In some embodiments of a compound of Formula (B), (B′), (IB), (C), (C′), (IC), (BC), (BC′), (CC), or (CC′), Ring C¹ is 5- to 12-membered heteroaryl. In some embodiments of a compound of Formula (B), (B′), (IB), (C), (C′), (IC), (BC), (BC′), (CC), or (CC′), Ring C¹ is 5- to 10-membered heteroaryl. In some embodiments of a compound of Formula (B), (B′), (IB), (C), (C′), (IC), (BC), (BC′), (CC), or (CC′), Ring C¹ is 5- to 6-membered heteroaryl. In some embodiments of a compound of Formula (B), (B′), (IB), (C), (C′), (IC), (BC), (BC′), (CC), or (CC′), Ring C¹ is 7- to 10-membered heteroaryl. In some embodiments of a compound of Formula (B), (B′), (IB), (C), (C′), (IC), (BC), (BC′), (CC), or (CC′), Ring C¹ is monocyclic heteroaryl. In some embodiments of a compound of Formula (B), (B′), (IB), (C), (C′), (IC), (BC), (BC′), (CC), or (CC′), Ring C¹ thiadiazolyl or phenyl. In some embodiments of a compound of Formula (B), (B′), (IB), (C), (C′), (IC), (BC), (BC′), (CC), or (CC′), Ring C¹ thiadiazolyl. In some embodiments of a compound of Formula (B), (B′), (IB), (C), (C′), (IC), (BC), (BC′), (CC), or (CC′), Ring C¹ phenyl.

In some embodiments of a compound of Formula (B), (B′), (IB), (C), (C′), (IC), (BC), (BC′), (CC), or (CC′), Ring C¹ is bicyclic heteroaryl.

In some embodiments of a compound of Formula (B), (B′), (IB), (C), (C′), (IC), (BC), (BC′), (CC), or (CC′), Ring C¹ is benzothiazolyl or imidazothiadiazolyl.

In some embodiments of a compound of Formula (B), (B′), (IB), (C), (C′), (IC), (BC), (BC′), (CC), or (CC′),

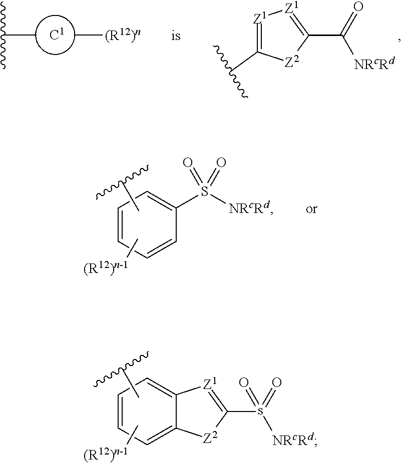

wherein each Z¹ is independently —N— or —CR¹²′—;
Z² is —O—, —S—, or —NR¹²″—;
each R¹²′ is independently hydrogen, halogen, —CN, —OH, —OR^{a}, —NR^{c}R^{d}, C₁-C₆alkyl, C₁-C₆haloalkyl, cycloalkyl, or heterocycloalkyl; and
R¹²″ is hydrogen, —S(═O)R^{a}, —S(═O)₂R^{a}, —S(═O)₂NR^{c}R^{d}, —C(═O)R^{a}, —C(═O)OR^{b}, —C(═O)NR^{c}R^{d}, C₁-C₆alkyl, C₁-C₆haloalkyl, cycloalkyl, or heterocycloalkyl.

In some embodiments, Z² is —O— or —S—. In some embodiments, Z² is —S—. In some embodiments, each Z¹ is —N—.

In some embodiments of a compound of Formula (B), (B′), (IB), (C), (C′), (IC), (BC), (BC′), (CC), or (CC′),

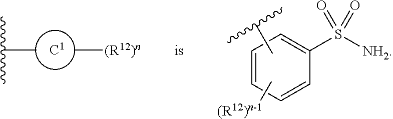

In some embodiments of a compound of Formula (B), (B′), (IB), (C), (C′), (IC), (BC), (BC′), (CC), or (CC′),

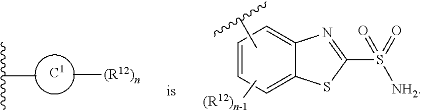

In some embodiments of a compound of Formula (B), (B′), (IB), (C), (C′), (IC), (BC), (BC′), (CC), or (CC′), each R¹² is independently halogen, —CN, —OH, —OR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, or C₁-C₆haloalkyl.

In some embodiments of a compound of Formula (B), (B′), (IB), (C), (C′), (IC), (BC), (BC′), (CC), or (CC′), each R¹² is independently halogen, —CN, —OH, —OR^{a}, —S(═O)R^{a}, —S(═O)₂R^{a}, —NR^{c}R^{d}, —C(═O)R^{a}, —C(═O)OR^{b}, —C(═O)NR^{c}R^{d}, C₁-C₆alkyl, or C₁-C₆haloalkyl.

In some embodiments of a compound of Formula (B), (B′), (IB), (C), (C′), (IC), (BC), (BC′), (CC), or (CC′), each R¹² is independently halogen, —S(═O)₂NR^{c}R^{d}, or —NR^{c}R^{d}.

In some embodiments of a compound of Formula (B), (B′), (IB), (C), (C′), (IC), (BC), (BC′), (CC), or (CC′), each R¹² is independently halogen or —NR^{c}R^{d}. In some embodiments of a compound of Formula (B), (B′), (IB), (C), (C′), (IC), (BC), (BC′), (CC), or (CC′), one of R¹² is —S(═O)₂NR^{c}R^{d}.

In some embodiments of a compound of Formula (B), (B′), (IB), (C), (C′), (IC), (BC), (BC′), (CC), or (CC′), n is 0, 1, 2, 3, 4, or 5. In some embodiments of a compound of Formula (B), (B′), (IB), (C), (C′), (IC), (BC), (BC′), (CC), or (CC′), n is 0, 1, 2, 3, or 4. In some embodiments of a compound of Formula (B), (B′), (IB), (C), (C′), (IC), (BC), (BC′), (CC), or (CC′), n is 0, 1, 2, or 3. In some embodiments of a compound of Formula (B), (B′), (IB), (C), (C′), (IC), (BC), (BC′), (CC), or (CC′), n is 0, 1, or 2. In some embodiments of a compound of Formula (B), (B′), (IB), (C), (C′), (IC), (BC), (BC′), (CC), or (CC′), n is 0 or 1.

In some embodiments of a compound of Formula (B), (B′), (IB), (C), (C′), (IC), (BC), (BC′), (CC), or (CC′),

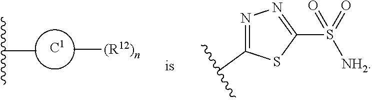

In some embodiments of a compound of Formula (B), (B′), (IB), (C), (C′), (IC), (BC), (BC′), (CC), or (CC′),

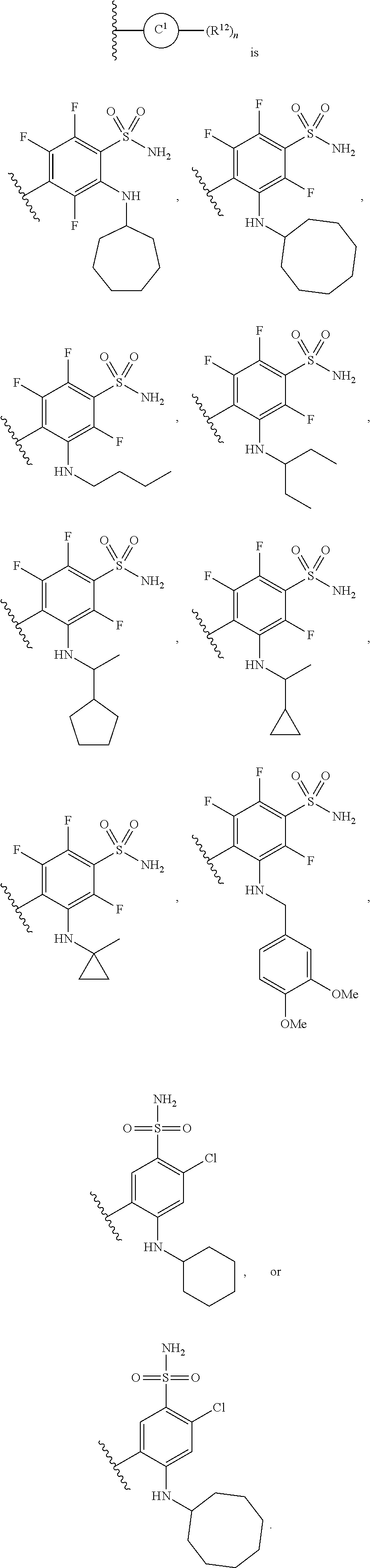

In some embodiments of a compound of Formula (B), (B′), (IB), (C), (C′), (IC), (BC), (BC′), (CC), or (CC′),

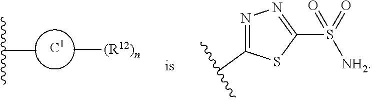

In some embodiments of a compound of Formula (B), (B′), (IB), (C), (C′), (IC), (BC), (BC′), (CC), or (CC′),

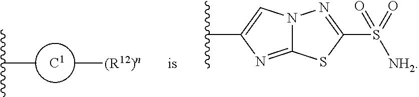

In some embodiments of a compound of Formula (B), (B′), (IIB), (C), (C′), (IIC), (BC), (BC′), (CC), or (CC′), W² is absent, —C(═O)—, or —NR²¹C(═O)—.

In some embodiments of a compound of Formula (B), (B′), (IIB), (C), (C′), (IIC), (BC), (BC′), (CC), or (CC′), W² is —C(═O)—.

In some embodiments of a compound of Formula (B), (B′), (IIB), (C), (C′), (IIC), (BC), (BC′), (CC), or (CC′), W² is absent. In some embodiments of a compound of Formula (B), (B′), (IIB), (C), (C′), (IIC), (BC), (BC′), (CC), or (CC′), W² is —C(═O)—.

In some embodiments of a compound of Formula (B), (B′), (IIB), (C), (C′), (IIC), (BC), (BC′), (CC), or (CC′), X² is C₁-C₆alkylene optionally substituted with one or more R.

In some embodiments of a compound of Formula (B), (B′), (IIB), (C), (C′), (IIC), (BC), (BC′), (CC), or (CC′), X² is C₁-C₄alkylene optionally substituted with one or more R.

In some embodiments of a compound of Formula (B), (B′), (IIB), (C), (C′), (IIC), (BC), (BC′), (CC), or (CC′), X² is —CH₂—.

In some embodiments of a compound of Formula (B), (B′), (IIB), (C), (C′), (IIC), (BC), (BC′), (CC), or (CC′), X² is —CH₂CH[C(═O)OH]— or —CH₂CH[C(═O)NH₂]—.

In some embodiments of a compound of Formula (B), (B′), (IIB), (C), (C′), (IIC), (BC), (BC′), (CC), or (CC′), X² is —CH₂CH₂CH [(C═O)OH]— or —CH₂CH₂CH [C(═O)NH₂]—.

In embodiments of a compound of Formula (B), (B′), (IIB), (C), (C′), (IIC), (BC), (BC′), (CC), or (CC′), X² is X² is optionally substituted with one or more R, and wherein one or two carbon atoms of the C₁-C₁₀alkylene are replaced with —S—, —S(═O)—, —S(═O)₂—, —O—, or —NR²¹—.

In some embodiments of a compound of Formula (B), (B′), (IIB), (C), (C′), (IIC), (BC), (BC′), (CC), or (CC′), Y² is absent, —O—, —S—, —NR²¹—, —C(═O)—, —C(═O)NR²¹—, or —NR²¹C(═O)—.

In some embodiments of a compound of Formula (B), (B′), (IIB), (C), (C′), (IIC), (BC), (BC′), (CC), or (CC′), Y² is absent, —NR²¹—, or —NR²¹C(═O)—.

In some embodiments of a compound of Formula (B), (B′), (IIB), (C), (C′), (IIC), (BC), (BC′), (CC), or (CC′), Y² is —NR²¹C(═O)—.

In some embodiments of a compound of Formula (B), (B′), (IIB), (C), (C′), (IIC), (BC), (BC′), (CC), or (CC′),

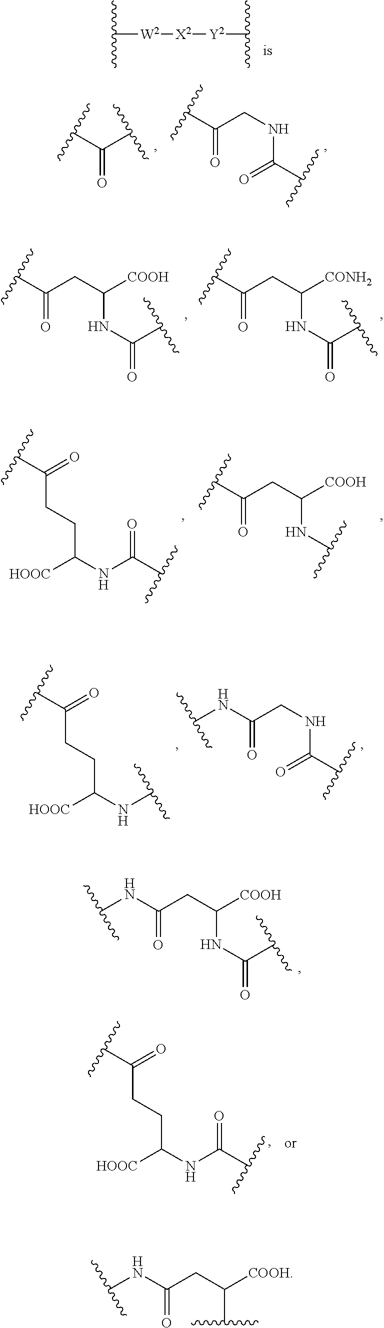

In some embodiments of a compound of Formula (B), (B′), (IIB), (C), (C′), (IIC), (BC), (BC′), (CC), or (CC′),

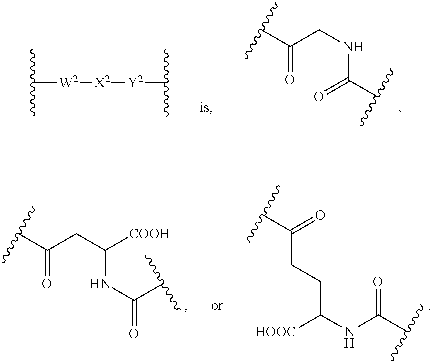

In some embodiments of a compound of Formula (B), (B′), (IIB), (C), (C′), (IIC), (BC), (BC′), (CC), or (CC′), S² is absent, —O—, —S—, —NR²¹—, —C(═O)—, —C(═O)NR²¹—, or —NR²¹C(═O)—.

In some embodiments of a compound of Formula (B), (B′), (IIB), (C), (C′), (IIC), (BC), (BC′), (CC), or (CC′), S² is absent, —NR²¹—, —C(═O)—, or —NR²¹C(═O)—.

In some embodiments of a compound of Formula (B), (B′), (IIB), (C), (C′), (IIC), (BC), (BC′), (CC), or (CC′), S² is —C(═O)— or —NR²¹C(═O)—.

In some embodiments of a compound of Formula (B), (B′), (IIB), (C), (C′), (IIC), (BC), (BC′), (CC), or (CC′), T² is absent or C₁-C₆alkylene optionally substituted with one or more R.

In some embodiments of a compound of Formula (B), (B′), (IIB), (C), (C′), (IIC), (BC), (BC′), (CC), or (CC′), T² is C₁-C₆alkylene optionally substituted with one or more R.

In some embodiments of a compound of Formula (B), (B′), (IIB), (C), (C′), (IIC), (BC), (BC′), (CC), or (CC′), T² is —CH₂CH₂—.

In some embodiments of a compound of Formula (B), (B′), (IIB), (C), (C′), (IIC), (BC), (BC′), (CC), or (CC′), T² is —CH₂CH₂CH₂—.

In some embodiments of a compound of Formula (B), (B′), (IIB), (C), (C′), (IIC), (BC), (BC′), (CC), or (CC′),

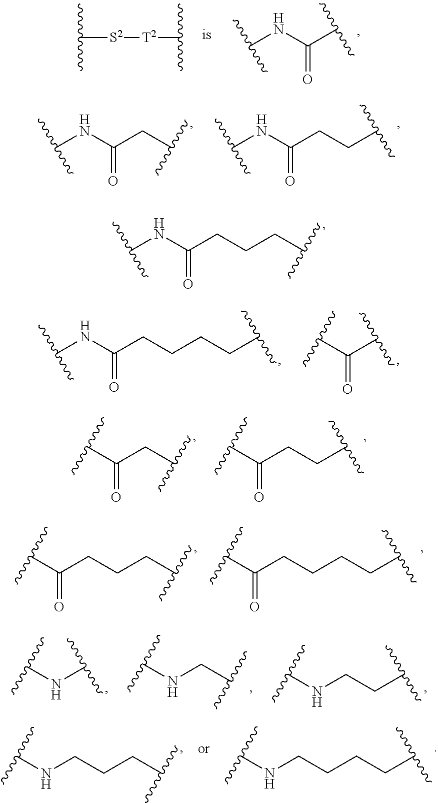

In some embodiments of a compound of Formula (B), (B′), (IIB), (C), (C′), (IIC), (BC), (BC′), (CC), or (CC′),

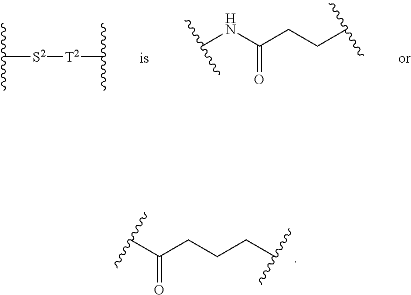

In some embodiments of a compound of Formula (B), (B′), (IIB), (C), (C′), (IIC), (BC), (BC′), (CC), or (CC′), B² is absent.

In embodiments of a compound of Formula (B), (B′), (IIB), (C), (C′), (IIC), (BC), (BC′), (CC), or (CC′), B² is —N(R²³)₂. In embodiments of a compound of Formula (B), (B′), (IIB), (C), (C′), (IIC), (BC), (BC′), (CC), or (CC′), B² is —CF₃.

In some embodiments of a compound of Formula (B), (B′), (IIB), (C), (C′), (IIC), (BC), (BC′), (CC), or (CC′), B² is cycloalkyl, heterocycloalkyl, aryl, or heteroaryl; wherein the cycloalkyl, heterocycloalkyl, aryl, and heteroaryl is optionally substitued with one or more R²⁴.

In some embodiments of a compound of Formula (B), (B′), (IIB), (C), (C′), (IIC), (BC), (BC′), (CC), or (CC′), B² is cycloalkyl optionally substitued with one or more R²⁴.

In some embodiments of a compound of Formula (B), (B′), (IIB), (C), (C′), (IIC), (BC), (BC′), (CC), or (CC′), B² is monocyclic cycloalkyl optionally substitued with one or more R²⁴.

In some embodiments of a compound of Formula (B), (B′), (IIB), (C), (C′), (IIC), (BC), (BC′), (CC), or (CC′), B² is bicyclic cycloalkyl optionally substitued with one or more R²⁴.

In some embodiments of a compound of Formula (B), (B′), (IIB), (C), (C′), (IIC), (BC), (BC′), (CC), or (CC′), B² is phenyl optionally substitued with one or more R²⁴.

In some embodiments of a compound of Formula (B), (B′), (IIB), (C), (C′), (IIC), (BC), (BC′), (CC), or (CC′), B² is heteroaryl optionally substitued with one or more R²⁴.

In some embodiments of a compound of Formula (B), (B′), (IIB), (C), (C′), (IIC), (BC), (BC′), (CC), or (CC′), B² is 5- to 10-membered heteroaryl (e.g., monocyclic or bicyclic heteroaryl) optionally substitued with one or more R²⁴.

In some embodiments of a compound of Formula (B), (B′), (IIB), (C), (C′), (IIC), (BC), (BC′), (CC), or (CC′), B² is 5- to 6-membered heteroaryl optionally substitued with one or more R²⁴.

In some embodiments of a compound of Formula (B), (B′), (IIB), (C), (C′), (IIC), (BC), (BC′), (CC), or (CC′), B² is indazolyl, phenyl, pyridyl, pyridininyl, pyrazinyl, pyridazinyl, cyclopropyl, cyclohexyl, cyclobutyl, cycloheptyl, pyrazolyl, oxazolyl, triazolyl, spiroheptanyl, or bicycloheptanyl.

In some embodiments of a compound of Formula (B), (B′), (IIB), (C), (C′), (IIC), (BC), (BC′), (CC), or (CC′), each R²⁴ is independently halogen, —CN, —OH, —OR^{a}, —NRcR⁴, —SF₅, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; wherein each alkyl, cycloalkyl, heterocycloalkyl, aryl, and heteroaryl is optionally and independently substituted with one or more R.

In some embodiments of a compound of Formula (B), (B′), (IIB), (C), (C′), (IIC), (BC), (BC′), (CC), or (CC′), each R²⁴ is independently halogen, —OH, C₁-C₆alkyl, or C₁-C₆haloalkyl.

In some embodiments of a compound of Formula (B), (B′), (IIB), (C), (C′), (IIC), (BC), (BC′), (CC), or (CC′), B² is

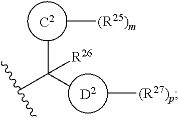

wherein:
Ring C² is cycloalkyl, heterocycloalkyl, aryl, or heteroaryl;
each R²⁵ is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
m is 0, 1, 2, 3, or 4;
R²⁶ is hydrogen, C₁-C₆alkyl, C₁-C₆haloalkyl, C₁-C₆hydroxyalkyl, C₁-C₆aminoalkyl, or C₁-C₆heteroalkyl;
Ring D² is cycloalkyl, heterocycloalkyl, aryl, or heteroaryl;
each R²⁷ is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
p is 0, 1, 2, 3, or 4.

In some embodiments of a compound of Formula (B), (B′), (IIB), (C), (C′), (IIC), (BC), (BC′), (CC), or (CC′), Ring C² is phenyl.

In some embodiments of a compound of Formula (B), (B′), (IIB), (C), (C′), (IIC), (BC), (BC′), (CC), or (CC′), each R²⁵ is independently halogen, —CN, —OH, —OR^{a}, —SF₅, —SH, —SR^{a}, —NR^{c}R^{d}, C₁-C₆alkyl, C₁-C₆haloalkyl, or C₁-C₆hydroxyalkyl. In some embodiments of a compound of Formula (B), (B′), (IIB), (C), (C′), (IIC), (BC), (BC′), (CC), or (CC′), each R²⁵ is independently halogen, —CN, —OH, —OR^{a}, —NR^{c}R^{d}, or C₁-C₆alkyl. In some embodiments of a compound of Formula (B), (B′), (IIB), (C), (C′), (IIC), (BC), (BC′), (CC), or (CC′), each R²⁵ is independently —OH.

In some embodiments of a compound of Formula (B), (B′), (IIB), (C), (C′), (IIC), (BC), (BC′), (CC), or (CC′), m is 0 or 1. In some embodiments of a compound of Formula (B), (B′), (IIB), (C), (C′), (IIC), (BC), (BC′), (CC), or (CC′), m is 0, 1, or 2. In some embodiments of a compound of Formula (B), (B′), (IIB), (C), (C′), (IC), (BC), (BC′), (CC), or (CC′), m is 0. In some embodiments of a compound of Formula (B), (B′), (IIB), (C), (C′), (IIC), (BC), (BC′), (CC), or (CC′), m is 1. In some embodiments of a compound of Formula (B), (B′), (IIB), (C), (C′), (IIC), (BC), (BC′), (CC), or (CC′), m is 2. In some embodiments of a compound of Formula (B), (B′), (IIB), (C), (C′), (IIC), (BC), (BC′), (CC), or (CC′), m is 3.

In some embodiments of a compound of Formula (B), (B′), (IIB), (C), (C′), (IIC), (BC), (BC′), (CC), or (CC′), Ring D² is phenyl.

In some embodiments of a compound of Formula (B), (B′), (IIB), (C), (C′), (IIC), (BC), (BC′), (CC), or (CC′), each R²⁷ is independently halogen, —CN, —OH, —OR^{a}, —SF₅, —SH, —SR^{a}, —NR^{c}R^{d}, C₁-C₆alkyl, C₁-C₆haloalkyl, or C₁-C₆hydroxyalkyl. In some embodiments of a compound of Formula (B), (B′), (IIB), (C), (C′), (IIC), (BC), (BC′), (CC), or (CC′), each R²⁷ is independently halogen, —CN, —OH, —OR^{a}, —NR^{c}R^{d}, or C₁-C₆alkyl. In some embodiments of a compound of Formula (B), (B′), (IIB), (C), (C′), (IIC), (BC), (BC′), (CC), or (CC′), each R²⁷ is independently —OH.

In some embodiments of a compound of Formula (B), (B′), (IIB), (C), (C′), (IIC), (BC), (BC′), (CC), or (CC′), p is 0 or 1. In some embodiments of a compound of Formula (B), (B′), (IIB), (C), (C′), (IIC), (BC), (BC′), (CC), or (CC′), p is 0, 1, or 2. In some embodiments of a compound of Formula (B), (B′), (IIB), (C), (C′), (IIC), (BC), (BC′), (CC), or (CC′), p is 0. In some embodiments of a compound of Formula (B), (B′), (IIB), (C), (C′), (IIC), (BC), (BC′), (CC), or (CC′), p is 1. In some embodiments of a compound of Formula (B), (B′), (IIB), (C), (C′), (IIC), (BC), (BC′), (CC), or (CC′), p is 2. In some embodiments of a compound of Formula (B), (B′), (IIB), (C), (C′), (IIC), (BC), (BC′), (CC), or (CC′), p is 3.

In some embodiments of a compound of Formula (B), (B′), (IIB), (C), (C′), (IIC), (BC), (BC′), (CC), or (CC′), R²⁶ is hydrogen or C₁-C₆alkyl. In some embodiments of a compound of Formula (B), (B′), (IIB), (C), (C′), (IIC), (BC), (BC′), (CC), or (CC′), R²⁶ is C₁-C₆alkyl.

In some embodiments of a compound of Formula (B), (B′), (IIB), (C), (C′), (IIC), (BC), (BC′), (CC), or (CC′), B² is

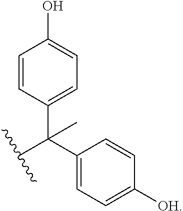 [SMILES: *C(C)(c1ccc(O)cc1)c1ccc(O)cc1]

In some embodiments of a compound of Formula (B), (B′), (IIB), (C), (C′), (IIC), (BC), (BC′), (CC), or (CC′), R¹¹ is hydrogen or C₁-C₆alkyl. In some embodiments of a compound of Formula (B), (B′), (IIB), (C), (C′), (IIC), (BC), (BC′), (CC), or (CC′), R¹¹ is hydrogen.

In some embodiments of a compound of Formula (A′), (A), (B), (B′), (C), (C′), (AC), (AC′), (BC), (BC′), (CC), or (CC′), TL₁ and TL₂ bind to different sites of the same carbonic anhydrase IX protein. In some embodiments of a compound of Formula (A′), (A), (B), (B′), (C), (C′), (AC), (AC′), (BC), (BC′), (CC), or (CC′), TL₁ is a targeting moiety that binds to a carbonic anhydrase IX protein at a first binding site. In some embodiments of a compound of Formula (A′), (A), (B), (B′), (C), (C′), (AC), (AC′), (BC), (BC′), (CC), or (CC′), TL is a targeting moiety that binds to a carbonic anhydrase IX protein at a site that is different from the binding site of TL₂. In some embodiments of a compound of Formula (A′), (A), (B), (B′), (C), (C′), (AC), (AC), (BC), (BC′), (CC), or (CC′), TL₂ is a targeting moiety that binds to carbonic anhydrase IX protein at a second binding site. In some embodiments of a compound of Formula (A′), (A), (B), (B′), (C), (C′), (AC), (AC), (BC), (BC′), (CC), or (CC′), TL₂ is a targeting moiety that binds to a carbonic anhydrase IX protein at a site that is different from the binding site of TL₁.

In some embodiments of a compound of Formula (A), (B), (C), (AC), (BC), or (CC), s is 0.

In some embodiments of a compound of Formula (A), (B), (C), (AC), (BC), or (CC), s is 1.

In some embodiments of a compound of Formula (A), (B), (C), (AC), (BC), or (CC), the linker comprises 1 to 30 intervening atoms selected from C, N, O, S, and Si between the metal chelator (CL) and the targeting ligand (TL).

In some embodiments of a compound of Formula (A), (B), (C), (AC), (BC), or (CC), the linker comprises 1 to 20 intervening atoms selected from C, N, O, S, and Si between the metal chelator (CL) and the targeting ligand (TL).

In some embodiments of a compound of Formula (A), (B), (C), (AC), (BC), or (CC), the linker comprises 1 to 10 intervening atoms selected from C, N, O, S, and Si between the metal chelator (CL) and the targeting ligand (TL).

In some embodiments of a compound of Formula (A), (B), (C), (AC), (BC), or (CC), the intervening atoms comprise C, N, or O. In some embodiments of a compound of Formula (A), (B), (C), (AC), (BC), or (CC), the intervening atoms comprise at least one N atom.

In some embodiments of a compound of Formula (A), (B), (C), (AC), (BC), or (CC), the intervening atoms comprise at least one O atom.

In some embodiments of a compound of Formula (A), (B), (C), (AC), (BC), or (CC), the linker comprises one or more amino acid residues.

In some embodiments of a compound of Formula (A), (B), (C), (AC), (BC), or (CC), the linker comprises one or more PEG moieties. In some embodiments of a compound of Formula (A), (B), (C), (AC), (BC), or (CC), the linker comprises between 2 and 8 PEG moieties. In some embodiments of a compound of Formula (A), (B), (C), (AC), (BC), or (CC), the linker comprises 2 PEG moieties. In some embodiments of a compound of Formula (A), (B), (C), (AC), (BC), or (CC), the linker comprises 8 PEG moieties

In some embodiments of a compound of Formula (A), (B), (C), (AC), (BC), or (CC), the linker L^{A} has a structure of Formula (L-1)

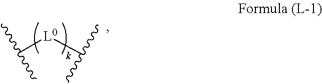

wherein:
each L⁰ is independently —O—, —NR^{L}—, —N(R^{L})₂O—, —OP(═O)(OR^{L})O—, —S—, —S(═O)—, —S(═O)₂—, —CH—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S)NR^{L}—, —CR^{L}═N—, —N═CR^{L}, —NR^{L}S(═O)₂—, —S(═O)₂NR^{L}—, —C(═O)NR^{L}S(═O)₂—, —S(═O)₂NR^{L}C(═O)—, cycloalkylene, heterocycloalkylene, arylene, heteroarylene, C₁-C₃₀alkylene, C₂-C₃₀alkenylene, C₂-C₃₀alkynylene, C₁-C₃₀heteroalkylene, —(C₁-C₃₀alkylene)-O—, —O—(C₁-C₃₀alkylene)-, —(C₁-C₃₀alkylene)-NR^{L}—, —NR^{L}—(C₁-C₃₀alkylene)-, —(C₁-C₃₀alkylene)-N(R^{L})₂⁺—, or —N(R^{L})₂⁺-(C₁-C₃₀alkylene)-; wherein each cycloalkylene, heterocycloalkylene, arylene, heteroarylene, alkylene, alkenylene, alkynylene, and heteroalkylene is optionally independently substituted with one or more R;
each R^{L} is independently hydrogen, C₁-C₄alkyl, C₁-C₄haloalkyl, C₁-C₄heteroalkyl, C₂-C₆alkenyl, C₂-C₆alkynyl, cycloalkyl, heterocycloalkyl, aryl, or heteroaryl;
k is 1-20;
each R is independently halogen, —CN, —OH, —SF₅, —SH, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, C₃-C₆cycloalkyl, or 3- to 6-membered heterocycloalkyl;
or two R on the same atom form an oxo.

In some embodiments of a compound of Formula (A), (B), (C), (AC), (BC), or (CC), the linker L^{A} has a structure of Formula (L-1)

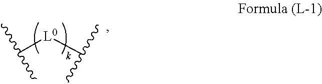

wherein:
each L⁰ is independently —O—, —NR^{L}—, —C(═O)—, —OC(═O)—, —C(═O)NR^{L}—, —NR^{L}C(═O)—, cycloalkylene, heterocycloalkylene, C₁-C₃₀alkylene, —(C₁-C₃₀alkylene)-O—, or —O—(C₁-C₃₀alkylene)-; wherein each cycloalkylene, heterocycloalkylene, and alkylene is optionally independently substituted with one or more R;
each R^{L} is independently hydrogen or C₁-C₄alkyl;
k is 1-20; and
R is independently —C(═O)OH.

In some embodiments of a compound of Formula (L-1), each L⁰ is independently —O—, —NR^{L}—, —C(═O)—, —C(═O)O—, —OC(═O)—, —C(═O)NR^{L}—, —NR^{L}C(═O)—, cycloalkylene, heterocycloalkylene, C₁-C₃₀alkylene, C₁-C₃₀heteroalkylene, —(C₁-C₃₀alkylene)-O—, —O—(C₁-C₃₀alkylene)-, —(C₁-C₃₀alkylene)-NR^{L}—, or —NR^{L}—(C₁-C₃₀alkylene)-; wherein each cycloalkylene, heterocycloalkylene, alkylene, and heteroalkylene is optionally independently substituted with one or more R.

In some embodiments of a compound of Formula (L-1), each L⁰ is independently —O—, —NR^{L}—, —C(═O)—, —C(═O)O—, —OC(═O)—, —C(═O)NR^{L}—, —NR^{L}C(═O)—, heterocycloalkylene, C₁-C₃₀alkylene, C₁-C₃₀heteroalkylene, —(C₁-C₃₀alkylene)-O—, or —O—(C₁-C₃₀alkylene)-; wherein each heterocycloalkylene, alkylene, and heteroalkylene is optionally independently substituted with one or more R.

In some embodiments of a compound of Formula (L-1), each L⁰ is independently —O—, —NR^{L}—, —C(═O)—, —OC(═O)—, —NR^{L}C(═O)—, heterocycloalkylene, C₁-C₃₀alkylene, C₁-C₃₀heteroalkylene, or —O—(C₁-C₃₀alkylene)-; wherein each heterocycloalkylene, alkylene, and heteroalkylene is optionally independently substituted with one or more R.

In some embodiments of a compound of Formula (L-1), each L⁰ is independently —O—, —NR^{L}—, —C(═O)—, —OC(═O)—, —NR^{L}C(═O)—, heterocycloalkylene, C₁-C₆alkylene, C₁-C₆heteroalkylene, or —O—(C₁-C₆alkylene)-; wherein each heterocycloalkylene, alkylene, and heteroalkylene is optionally independently substituted with one or more R.

In some embodiments of a compound of Formula (L-1), each L⁰ is independently —O—, —NR^{L}—, —C(═O)—, —OC(═O)—, —NR^{L}C(═O)—, heterocycloalkylene, C₁-C₄alkylene, C₁-C₄heteroalkylene, or —O—(C₁-C₄alkylene)-; wherein each heterocycloalkylene, alkylene, and heteroalkylene is optionally independently substituted with one or more R.

In some embodiments of a compound of Formula (L-1), each R^{L} is independently hydrogen or C₁-C₄alkyl. In some embodiments of a compound of Formula (L-1), each R^{L} is hydrogen.

In some embodiments of a compound of Formula (L-1), k is 1-15. In some embodiments of a compound of Formula (L-1), k is 1-14. In some embodiments of a compound of Formula (L-1), k is 1-13. In some embodiments of a compound of Formula (L-1), k is 1-12. In some embodiments of a compound of Formula (L-1), k is 1-11. In some embodiments of a compound of Formula (L-1), k is 1-10. In some embodiments of a compound of Formula (L-1), k is 1-9. In some embodiments of a compound of Formula (L-1), k is 1-8. In some embodiments of a compound of Formula (L-1), k is 1-7. In some embodiments of a compound of Formula (L-1), k is 1-6. In some embodiments of a compound of Formula (L-1), k is 1-5. In some embodiments of a compound of Formula (L-1), k is 1-4. In some embodiments of a compound of Formula (L-1), k is 1-3. In some embodiments of a compound of Formula (L-1), k is 1 or 2.

In some embodiments of a compound of Formula (A), (B), (C), (AC), (BC), or (CC), the linker L^{A} has a structure of Formula (L-1a),

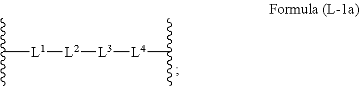

wherein:
L¹ is absent, —O—, —NR^{L}—, —N(R^{L})₂⁺—, —S—, —S(═O)—, —S(═O)₂—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}S(═O)₂—, —S(═O)₂NR^{L}—, —C(═O)NR^{L}S(═O)₂—, or —S(═O)₂NR^{L}C(═O)—;
L² is

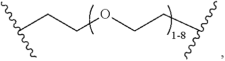

C₁-C₁₀alkylene, or heterocycloalkylene optionally substituted with one or more R:
L³ is absent or C₁-C₁₀alkylene; and
L⁴ is absent, —O—, —NR^{L}—, —N(R^{L})₂⁺—, —S—, —S(═O)—, —S(═O)₂—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}S(═O)₂—, —S(═O)₂NR^{L}—, —C(═O)NR^{L}S(═O)₂—, or —S(═O)₂NR^{L}C(═O)—.

In some embodiments of a compound of Formula (A), (B), (C), (AC), (BC), or (CC), the linker L^{A} has a structure of Formula (L-1a),

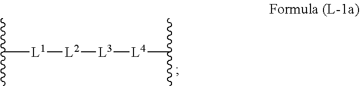

wherein:
L¹ is absent, —O—, or —NR^{L}—;
L² is

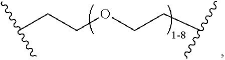

C₁-C₁₀alkylene, or heterocycloalkylene optionally substituted with one or more R:
L³ is absent or C₁-C₁₀alkylene; and
L⁴ is absent, —O—, —NR^{L}—, —C(═O)—, —C(═O)O—, —OC(═O)—, —C(═O)NR^{L}—, or —NR^{L}C(═O)—.

In some embodiments of a compound of Formula (L-1a), L¹ is absent, —O—, —NR^{L}—, —C(═O)—, —C(═O)O—, —OC(═O)—, —C(═O)NR^{L}—, or —NR^{L}C(═O)—. In some embodiments of a compound of Formula (L-1a), L¹ is absent, —O—, or —NR^{L}—. In some embodiments of a compound of Formula (L-1a), L¹ is absent or —NR^{L}—. In some embodiments of a compound of Formula (L-1a), L¹ is absent or —NH—.

In some embodiments of a compound of Formula (L-1a), L² is

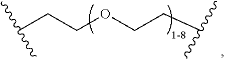

C₁-C₆alkylene, or heterocycloalkylene optionally substituted with one or more R. In some embodiments of a compound of Formula (L-1a), L² is

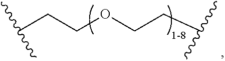

C₁-C₄alkylene, or heterocycloalkylene optionally substituted with one or more R. In some embodiments of a compound of Formula (L-1a), L² is

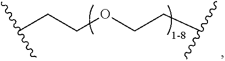

or heterocycloalkylene optionally substituted with one or more R.

In some embodiments of a compound of Formula (L-1a), L² is

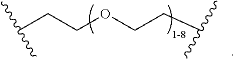

In some embodiments of a compound of Formula (L-1a), L² is

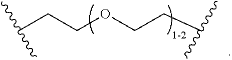

In some embodiments of a compound of Formula (L-1a), L² is

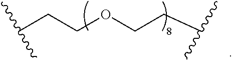

In some embodiments of a compound of Formula (L-1a), L² is

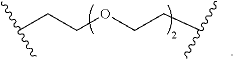

In some embodiments of a compound of Formula (L-1a), L² is C₁-C₁₀alkylene. In some embodiments of a compound of Formula (L-1a), L² is C₁-C₆alkylene. In some embodiments of a compound of Formula (L-1a), L² is C₁-C₄alkylene.

In some embodiments of a compound of Formula (L-1a), L² is heterocycloalkylene optionally substituted with one or more R. In some embodiments of a compound of Formula (L-1a), L² is monocyclic heterocycloalkylene optionally substituted with one or more R. In some embodiments of a compound of Formula (L-1a), L² is bicyclic heterocycloalkylene optionally substituted with one or more R. In some embodiments of a compound of Formula (L-1a), L² is piperidinylene, azetidinylene, or diazaspiroheptanylene; each optionally substituted with one or more R.

In some embodiments of a compound of Formula (L-1a), L³ is absent. In some embodiments of a compound of Formula (L-1a), L³ is —CH₂—.

In some embodiments of a compound of Formula (L-1a), L⁴ is absent, —O—, —NR^{L}—, —C(═O)—, —C(═O)O—, —OC(═O)—, —C(═O)NR^{L}—, or —NR^{L}C(═O)—. In some embodiments of a compound of Formula (L-1a), L⁴ is absent, —C(═O)—, —OC(═O)—, or —NR^{L}C(═O)—. In some embodiments of a compound of Formula (L-1a), L⁴ is absent, —C(═O)—, —OC(═O)—, or —NHC(═O)—.

In some embodiments of a compound of Formula (A), (B), (C), (AC), (BC), or (CC), the linker L^{A} is

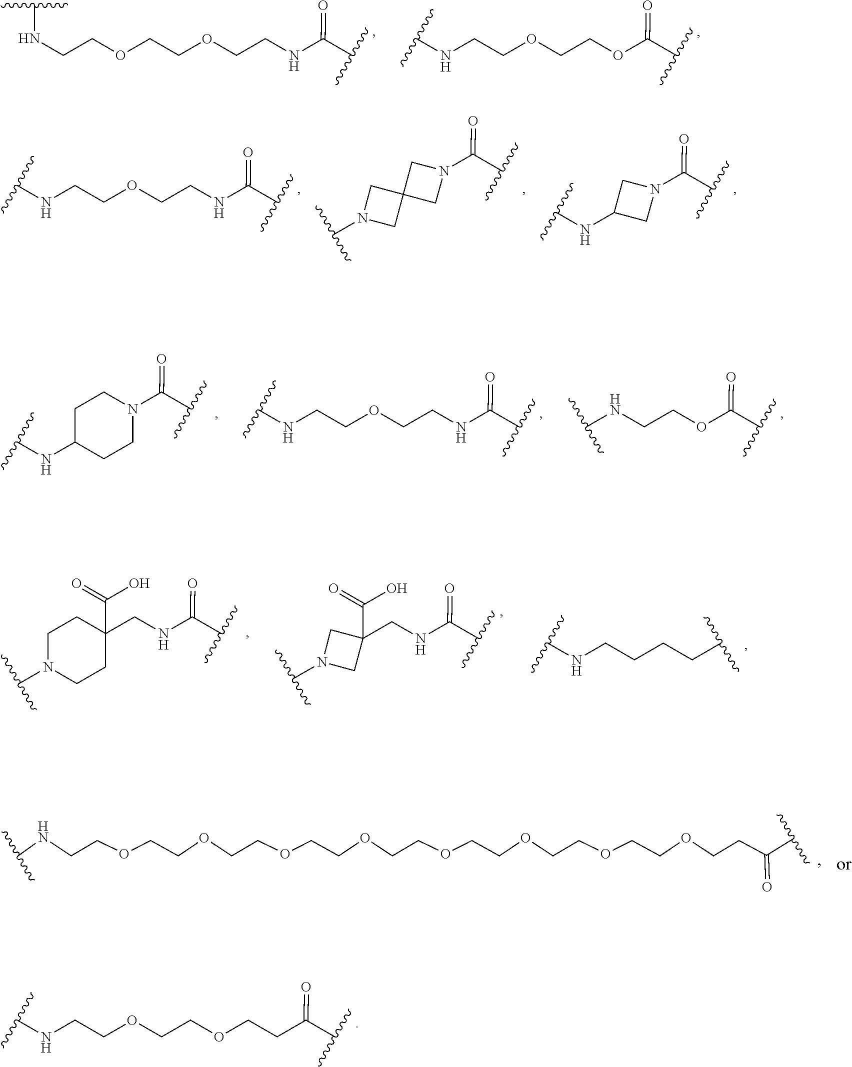

In some embodiments of a compound of Formula (A), (B), (C), (AC), (BC), or (CC), the linker L^{A} has the structure of Formula (L-2):

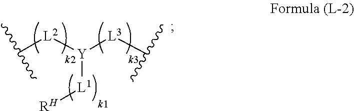

wherein:
each L¹, L², and L³ is independently —O—, —NR^{L}—, —N(R^{L})₂⁺—, —OP(═O)(OR^{L})O—, —S—, —S(═O)—, —S(═O)₂—, ═CH—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S)NR^{L}—, —CR^{L}═N—, —N═CR^{L}, —NR^{L}S(═O)₂—, —S(═O)₂NR^{L}—, —C(═O)NR^{L}S(═O)₂—, —S(═O)₂NR^{L}C(═O)—, C₃-C₁₅ cycloalkylene, C₁-C₁₂ heterocycloalkylene, arylene, heteroarylene, C₁-C₃₀alkylene, C₂-C₃₀alkenylene, C₂-C₃₀alkynylene, C₁-Csoheteroalkylene, —(C₁-C₃₀alkylene)-O—, —O—(C₁-C₃₀alkylene)-, —(C₁-C₃₀alkylene)-NR^{L}—, —NR^{L}—(C₁-C₃₀alkylene)-, —(C₁-C₃₀alkylene)-N(R^{L})₂⁺—, or —N(R^{L})₂⁺—(C₁-C₃₀ alkylene)-; wherein each cycloalkylene, heterocycloalkylene, arylene, heteroarylene, alkylene, alkenylene, alkynylene, and heteroalkylene is optionally independently substituted with one or more R;
R^{H} is a hydrophobic moiety;
Y is N or CR^{L};
each R^{L} is independently hydrogen, C₁-C₄alkyl, C₁-C₄haloalkyl, C₁-C₄heteroalkyl, C₂-C₆alkenyl, C₂-C₆alkynyl, cycloalkyl, heterocycloalkyl, aryl, or heteroaryl;
k1 is 0-20;
k2 is 0-15;
k3 is 0-15; and
each R is independently halogen, —CN, —OH, —SF₅, —SH, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, C₃-C₆cycloalkyl, or 3- to 6-membered heterocycloalkyl;
or two R on the same atom form an oxo.

In some embodiments of a compound of Formula (A), (B), (C), (AC), (BC), or (CC), the linker L^{A} has the structure of Formula (L-2):

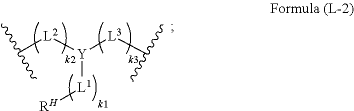

wherein:
each L¹, L², and L³ is independently —O—, —NR^{L}—, or —C(═O)—;
R^{H} is a hydrophobic moiety;
Y is N or CR^{L};
each R^{L} is independently hydrogen or C₁-C₄alkyl;
k1 is 0-20;
k2 is 0-15; and
k3 is 0-15.

In some embodiments of a compound of Formula (L-2),
each L² and L³ is independently —O—, —NR^{L}—, —N(R^{L})₂⁺—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—O—(CH₂CH₂)—O—(CH₂CH₂), or C₁-C₆alkylene optionally independently substituted with one or more R;
R^{L} is hydrogen or C₁-C₄ alkyl;
k2 is 1-5; and
k3 is 1-5.

In some embodiments of a compound of Formula (L-2),
each L¹ is independently —O—, —NR^{L}—, —N(R^{L})₂⁺—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —O—(CH₂CH₂), or C₁-C₆alkylene optionally independently substituted with one or more R;
R^{L} is hydrogen or C₁-C₄ alkyl;
k1 is 1-15; and
k2 is 1-15.

In some embodiments of a compound of Formula (L-2), each L² and L³ is independently —O—, —NR^{L}—, —N(R^{L})₂⁺—, —C(═O)—, —C(═O)O—, —OC(═O)—, —OC(═O)O—, —C(═O)NR^{L}—, —NRIC(═O)—, —OC(═O)NR^{L}—, —NR^{L}C(═O)O—, —NR^{L}C(═O)NR^{L}—, —O—(CH₂CH₂), or C₁-C₆alkylene optionally independently substituted with one or more R.

In some embodiments of a compound of Formula (L-2), each L² and L³ is independently —O—, —NR^{L}—, —C(═O)—, —C(═O)O—, —OC(═O)—, —C(═O)NR^{L}—, —NR^{L}C(═O)—, —O—(CH₂CH₂), or C₁-C₆alkylene optionally independently substituted with one or more R.

In some embodiments of a compound of Formula (L-2), each L² and L³ is independently —O—, —NR^{L}—, —C(═O)—, —O—(CH₂CH₂), or C₁-C₆alkylene optionally independently substituted with one or more R.

In some embodiments of a compound of Formula (L-2), each L² and L³ is independently —O—, —NR^{L}—, or —C(═O)—.

In some embodiments of a compound of Formula (L-2), each L² and L³ is independently —NH— or —C(═O)—.

In some embodiments of a compound of Formula (L-2), each L¹ is independently —O—, —NR^{L}—, —N(R^{L})₂⁺—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —O—(CH₂CH₂), or C₁-C₆alkylene optionally independently substituted with one or more R.

In some embodiments of a compound of Formula (L-2), each L¹ is independently —O—, —NR^{L}—, —C(═O)—, —C(═O)O—, —OC(═O)—, —C(═O)NR^{L}—, —NR^{L}C(═O)—, —O—(CH₂CH₂), or C₁-C₆alkylene optionally independently substituted with one or more R;

In some embodiments of a compound of Formula (L-2), each L¹ is independently —C(═O)NR^{L}—, —NR^{L}C(═O)—, —O—(CH₂CH₂), or C₁-C₆alkylene optionally independently substituted with one or more R;

In some embodiments of a compound of Formula (L-2), each L¹ is independently —C(═O)NH—, —NHC(═O)—, —O—(CH₂CH₂), or C₁-C₆alkylene optionally independently substituted with one or more R.

In some embodiments of a compound of Formula (L-2), R^{L} is hydrogen or C₁-C₄ alkyl. In some embodiments of a compound of Formula (L-2), R^{L} is hydrogen.

In some embodiments of a compound of Formula (L-2), k1 is 1-19. In some embodiments of a compound of Formula (L-2), k1 is 1-18. In some embodiments of a compound of Formula (L-2), k1 is 1-17. In some embodiments of a compound of Formula (L-2), k1 is 1-16. In some embodiments of a compound of Formula (L-2), k1 is 1-15. In some embodiments of a compound of Formula (L-2), k1 is 1-14. In some embodiments of a compound of Formula (L-2), k1 is 1-13. In some embodiments of a compound of Formula (L-2), k1 is 1-12. In some embodiments of a compound of Formula (L-2), k1 is 1-11. In some embodiments of a compound of Formula (L-2), k1 is 1-10. In some embodiments of a compound of Formula (L-2), k1 is 1-9. In some embodiments of a compound of Formula (L-2), k1 is 1-8. In some embodiments of a compound of Formula (L-2), k1 is 1-7. In some embodiments of a compound of Formula (L-2), k1 is 1-6. In some embodiments of a compound of Formula (L-2), k1 is 1-5. In some embodiments of a compound of Formula (L-2), k1 is 1-4. In some embodiments of a compound of Formula (L-2), k1 is 1-3. In some embodiments of a compound of Formula (L-2), k1 is 1 or 2.

In some embodiments of a compound of Formula (L-2), k2 is 1-14. In some embodiments of a compound of Formula (L-2), k2 is 1-13. In some embodiments of a compound of Formula (L-2), k2 is 1-12. In some embodiments of a compound of Formula (L-2), k2 is 1-11. In some embodiments of a compound of Formula (L-2), k2 is 1-10. In some embodiments of a compound of Formula (L-2), k2 is 1-9. In some embodiments of a compound of Formula (L-2), k2 is 1-8. In some embodiments of a compound of Formula (L-2), k2 is 1-7. In some embodiments of a compound of Formula (L-2), k2 is 1-6. In some embodiments of a compound of Formula (L-2), k2 is 1-5. In some embodiments of a compound of Formula (L-2), k2 is 1-4. In some embodiments of a compound of Formula (L-2), k2 is 1-3. In some embodiments of a compound of Formula (L-2), k2 is 1 or 2.

In some embodiments of a compound of Formula (L-2), k3 is 1-14. In some embodiments of a compound of Formula (L-2), k3 is 1-13. In some embodiments of a compound of Formula (L-2), k3 is 1-12. In some embodiments of a compound of Formula (L-2), k3 is 1-11. In some embodiments of a compound of Formula (L-2), k3 is 1-10. In some embodiments of a compound of Formula (L-2), k3 is 1-9. In some embodiments of a compound of Formula (L-2), k3 is 1-8. In some embodiments of a compound of Formula (L-2), k3 is 1-7. In some embodiments of a compound of Formula (L-2), k3 is 1-6. In some embodiments of a compound of Formula (L-2), k3 is 1-5. In some embodiments of a compound of Formula (L-2), k3 is 1-4. In some embodiments of a compound of Formula (L-2), k3 is 1-3. In some embodiments of a compound of Formula (L-2), k3 is 1 or 2.

In some embodiments of a compound of Formula (L-2),
R^{H} is a fatty acid chain, a C₆-C₁₀ alkyl chain, a sterol, an aryl, or a heteroaryl; wherein the aryl and heteroaryl is optionally substituted with one or more R; and
each R is independently halogen, —CN, —OH, —SF₅, —SH, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl) 2, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, C₃-C₆cycloalkyl, or 3- to 6-membered heterocycloalkyl.

In some embodiments of a compound of Formula (L-2), R^{H} is phenyl substituted with a halogen or a C₁-C₃ alkyl.

In some embodiments of a compound of Formula (A), (B), (C), (AC), (BC), or (CC), the linker L^{A} is

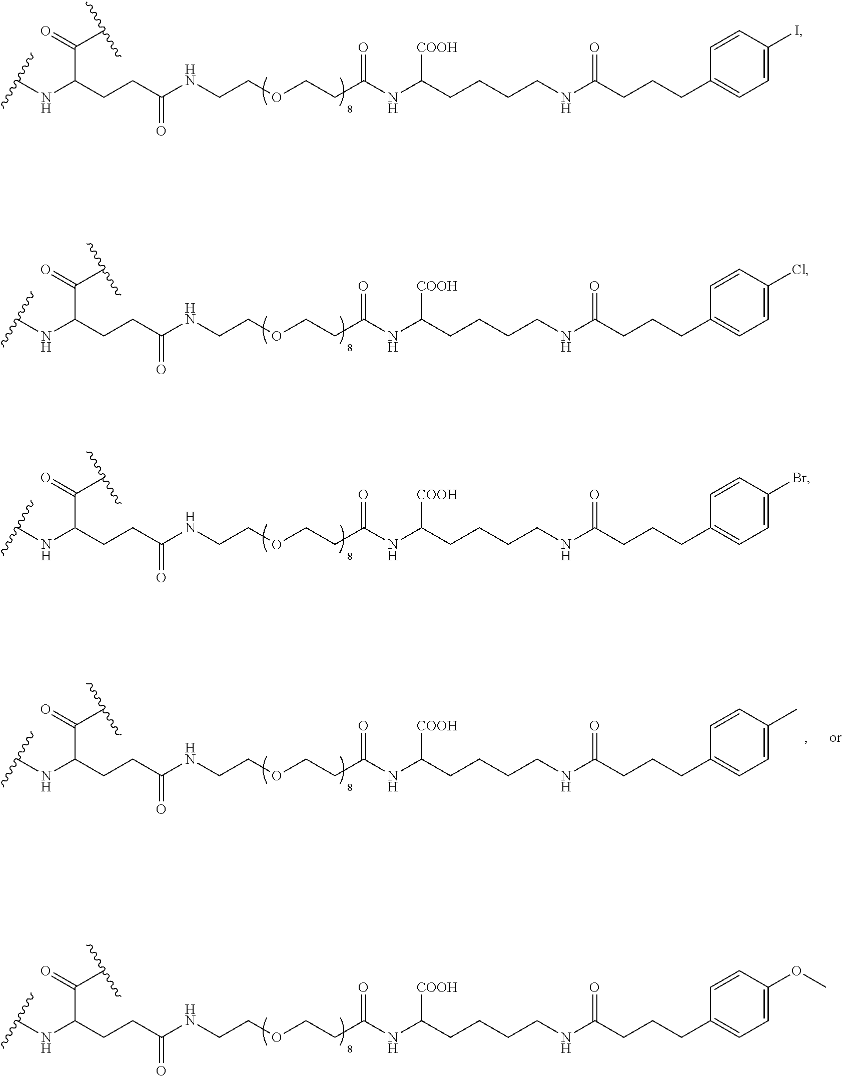

In some embodiments of a compound of Formula (A), (B), (C), (AC), (BC), or (CC), the linker L^{A} is

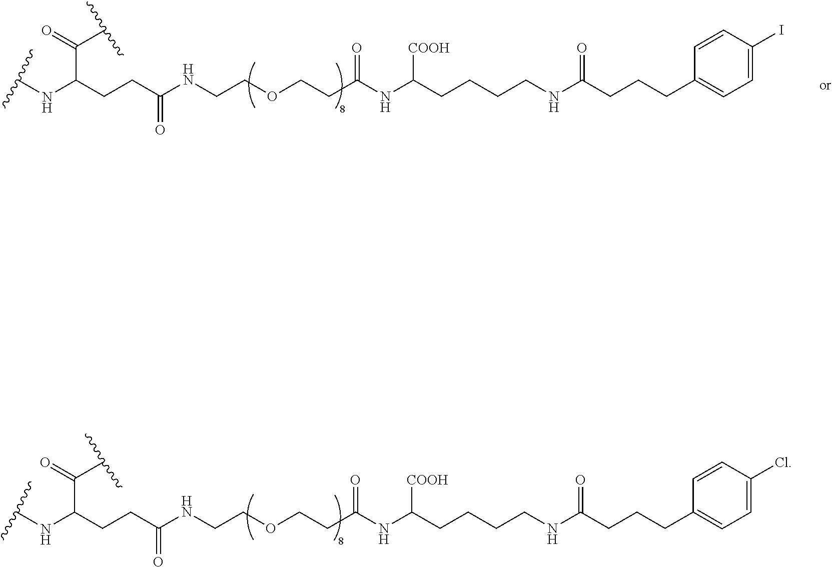

In some embodiments of a compound of Formula (A′), (B′), (C′), (AC′), (BC′), or (CC′), the linker L^{B} has the structure of Formula (L-3):

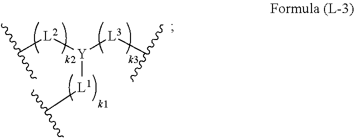

wherein:
each L¹, L², and L³ is independently —O—, —NR^{L}—, —N(R^{L})₂⁺—, —OP(═O)(OR^{L})O—, —S—, —S(═O)—, —S(═O)₂—, ═CH—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S)NR^{L}—, —CR^{L}═N—, —N═CR^{L}, —NR^{L}S(═O)₂—, —S(═O)₂NR^{L}—, —C(═O)NR^{L}S(═O)₂—, —S(═O)₂NR^{L}C(═O)—, C₃-C₁₅ cycloalkylene, C₁-C₁₂ heterocycloalkylene, arylene, heteroarylene, C₁-C₃₀alkylene, C₂-C₃₀alkenylene, C₂-C₃₀alkynylene, C₁-C₃₀heteroalkylene, —(C₁-C₃₀alkylene)-O—, —O—(C₁-C₃₀ alkylene)-, —(C₁-C₃₀alkylene)-NR^{L}—, —NR^{L}—(C₁-C₃₀alkylene)-, —(C₁-C₃₀alkylene)-N(R^{L})₂⁺—, or —N(R^{L})₂⁺—(C₁-C₃₀ alkylene)-; wherein each cycloalkylene, heterocycloalkylene, arylene, heteroarylene, alkylene, alkenylene, alkynylene, and heteroalkylene is optionally independently substituted with one or more R;
Y is N or CR^{L};
each R^{L} is independently hydrogen, C₁-C₄alkyl, C₁-C₄haloalkyl, C₁-C₄heteroalkyl, C₂-C₆alkenyl, C₂-C₆alkynyl, cycloalkyl, heterocycloalkyl, aryl, or heteroaryl;
k1 is 0-15;
k2 is 0-15;
k3 is 0-15; and
each R is independently halogen, —CN, —OH, —SF₅, —SH, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, C₃-C₆cycloalkyl, or 3- to 6-membered heterocycloalkyl;
or two R on the same atom form an oxo.

In some embodiments of a compound of Formula (A′), (B′), (C′), (AC′), (BC′), or (CC′), the linker L^{B} has the structure of Formula (L-3):

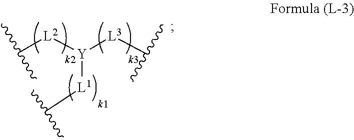

wherein:
each L¹, L², and L³ is independently —O—, —NR^{L}—, —C(═O)—, C₁-C₃₀alkylene, —(C₁-C₃₀alkylene)-O—, or —O—(C₁-C₃₀alkylene)-;
Y is N or CR^{L};
each R^{L} is independently hydrogen or C₁-C₄alkyl;
k1 is 0-15;
k2 is 0-15; and
k3 is 0-15.

In some embodiments of a compound of Formula (L-3), each L² and L³ is independently —O—, —NR^{L}—, —N(R^{L})₂⁺—, —C(═O)—, —C(═O)O—, —OC(═O)—, —OC(═O)O—, —C(═O)NR^{L}—, —NRIC(═O)—, —OC(═O)NR^{L}—, —NR^{L}C(═O)O—, —NR^{L}C(═O)NR^{L}—, —O—(CH₂CH₂), or C₁-C₆alkylene optionally independently substituted with one or more R.

In some embodiments of a compound of Formula (L-3), each L² and L³ is independently —O—, —NR^{L}—, —C(═O)—, —C(═O)O—, —OC(═O)—, —C(═O)NR^{L}—, —NR^{L}C(═O)—, —O—(CH₂CH₂), or C₁-C₆alkylene optionally independently substituted with one or more R.

In some embodiments of a compound of Formula (L-3), each L² and L³ is independently —O—, —NR^{L}—, —C(═O)—, —O—(CH₂CH₂), or C₁-C₆alkylene optionally independently substituted with one or more R.

In some embodiments of a compound of Formula (L-3), each L² and L³ is independently —C(═O)—, —O—(CH₂CH₂), or C₁-C₆alkylene optionally independently substituted with one or more R.

In some embodiments of a compound of Formula (L-3), each L² and L³ is independently —C(═O)—, —O—(CH₂CH₂), or C₁-C₆alkylene.

In some embodiments of a compound of Formula (L-3), each L² and L³ is independently —C(═O)— or C₁-C₆alkylene.

In some embodiments of a compound of Formula (L-3), each L¹ is independently —O—, —NR^{L}—, —N(R^{L})₂⁺—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —O—(CH₂CH₂), or C₁-C₆alkylene optionally independently substituted with one or more R.

In some embodiments of a compound of Formula (L-3), each L¹ is independently —O—, —NR^{L}—, —C(═O)—, —C(═O)O—, —OC(═O)—, —C(═O)NR^{L}—, —O—(CH₂CH₂), or C₁-C₆alkylene optionally independently substituted with one or more R.

In some embodiments of a compound of Formula (L-3), each L¹ is independently —O—, —NR^{L}—, —C(═O)—, —O—(CH₂CH₂), or C₁-C₆alkylene optionally independently substituted with one or more R.

In some embodiments of a compound of Formula (L-3), each L¹ is independently —NR^{L}—, —C(═O)—, or C₁-C₆alkylene optionally independently substituted with one or more R.

In some embodiments of a compound of Formula (L-3), each L¹ is independently —NR^{L}—.

In some embodiments of a compound of Formula (L-3), Y is N or CR^{L}. In some embodiments of a compound of Formula (L-3), Y is N. In some embodiments of a compound of Formula (L-3), Y is CR^{L}. In some embodiments of a compound of Formula (L-3), Y is CH.

In some embodiments of a compound of Formula (L-3), k1 is 1-15. In some embodiments of a compound of Formula (L-3), k1 is 1-14. In some embodiments of a compound of Formula (L-3), k1 is 1-13. In some embodiments of a compound of Formula (L-3), k1 is 1-12. In some embodiments of a compound of Formula (L-3), k1 is 1-11. In some embodiments of a compound of Formula (L-3), k1 is 1-10. In some embodiments of a compound of Formula (L-3), k1 is 1-9. In some embodiments of a compound of Formula (L-3), k1 is 1-8. In some embodiments of a compound of Formula (L-3), k1 is 1-7. In some embodiments of a compound of Formula (L-3), k1 is 1-6. In some embodiments of a compound of Formula (L-3), k1 is 1-5. In some embodiments of a compound of Formula (L-3), k1 is 1-4. In some embodiments of a compound of Formula (L-3), k1 is 1-3. In some embodiments of a compound of Formula (L-3), k1 is 1 or 2. In some embodiments of a compound of Formula (L-3), k1 is 0-2. In some embodiments of a compound of Formula (L-3), k1 is 0 or 1. In some embodiments of a compound of Formula (L-3), k1 is 0. In some embodiments of a compound of Formula (L-3), k1 is 1. In some embodiments of a compound of Formula (L-3), k1 is 2. In some embodiments of a compound of Formula (L-3), k1 is 3. In some embodiments of a compound of Formula (L-3), k1 is 4.

In some embodiments of a compound of Formula (L-3), k2 is 1-14. In some embodiments of a compound of Formula (L-3), k2 is 1-13. In some embodiments of a compound of Formula (L-3), k2 is 1-12. In some embodiments of a compound of Formula (L-3), k2 is 1-11. In some embodiments of a compound of Formula (L-3), k2 is 1-10. In some embodiments of a compound of Formula (L-3), k2 is 1-9. In some embodiments of a compound of Formula (L-3), k2 is 1-8. In some embodiments of a compound of Formula (L-3), k2 is 1-7. In some embodiments of a compound of Formula (L-3), k2 is 1-6. In some embodiments of a compound of Formula (L-3), k2 is 1-5. In some embodiments of a compound of Formula (L-3), k2 is 1-4. In some embodiments of a compound of Formula (L-3), k2 is 1-3. In some embodiments of a compound of Formula (L-3), k2 is 1 or 2.

In some embodiments of a compound of Formula (L-3), k3 is 1-14. In some embodiments of a compound of Formula (L-3), k3 is 1-13. In some embodiments of a compound of Formula (L-3), k3 is 1-12. In some embodiments of a compound of Formula (L-3), k3 is 1-11. In some embodiments of a compound of Formula (L-3), k3 is 1-10. In some embodiments of a compound of Formula (L-3), k3 is 1-9. In some embodiments of a compound of Formula (L-3), k3 is 1-8. In some embodiments of a compound of Formula (L-3), k3 is 1-7. In some embodiments of a compound of Formula (L-3), k3 is 1-6. In some embodiments of a compound of Formula (L-3), k3 is 1-5. In some embodiments of a compound of Formula (L-3), k3 is 1-4. In some embodiments of a compound of Formula (L-3), k3 is 1-3. In some embodiments of a compound of Formula (L-3), k3 is 1 or 2.

In some embodiments of a compound of Formula (A′), (B′), (C′), (AC′), (BC′), or (CC′), the linker L^{B} is

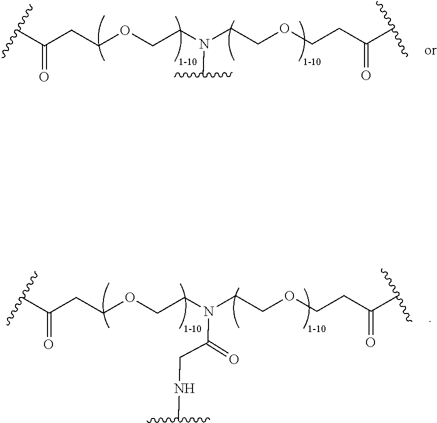

In some embodiments of a compound of Formula (A′), (B′), (C′), (AC′), (BC′), or (CC′), the linker L^{B} is

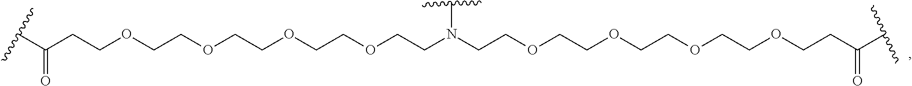 [SMILES: *C(=O)CCOCCOCCOCCOCCN(*)CCOCCOCCOCCOCCC(*)=O]

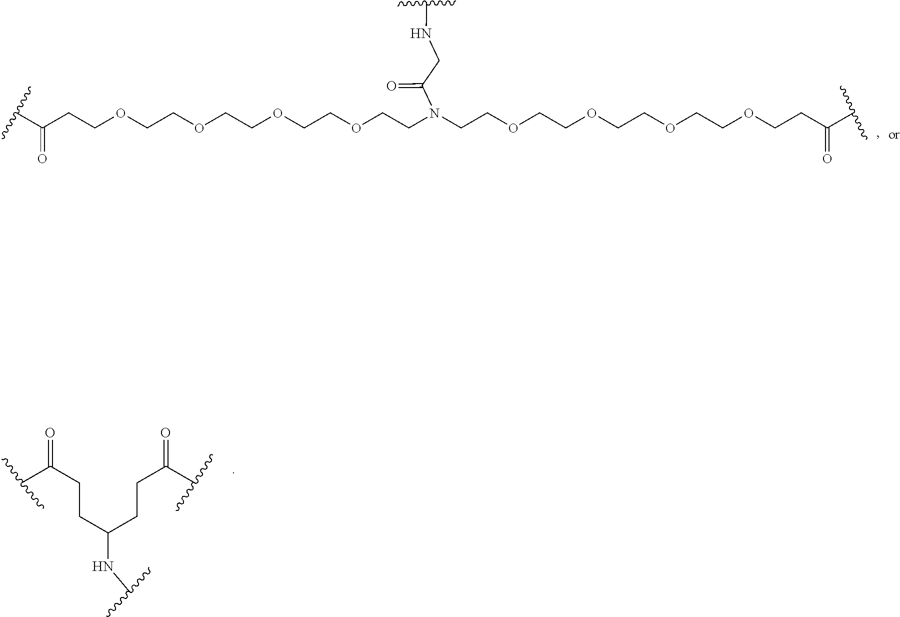 [SMILES: *NCC(=O)N(CCOCCOCCOCCOCCC(*)=O)CCOCCOCCOCCOCCC(*)=O ; *NC(CCC(*)=O)CCC(*)=O]

In some embodiments of a compound disclosed herein, each R^{a} is independently C₁-C₆alkyl, C₁-C₆haloalkyl, C₁-C₆hydroxyalkyl, C₁-C₆aminoalkyl, C₁-C₆heteroalkyl, -L-cycloalkyl, or -L-heterocycloalkyl, wherein each alkyl, cycloalkyl, and heterocycloalkyl is independently optionally substituted with one or more R. In some embodiments of a compound disclosed herein, each R^{a} is independently C₁-C₆alkyl, C₁-C₆haloalkyl, -L-cycloalkyl, or -L-heterocycloalkyl, wherein each alkyl, cycloalkyl, and heterocycloalkyl is independently optionally substituted with one or more R. In some embodiments of a compound disclosed herein, each R^{a} is independently C₁-C₆alkyl, C₁-C₆haloalkyl, C₁-C₆hydroxyalkyl, C₁-C₆aminoalkyl, or C₁-C₆heteroalkyl, wherein each alkyl is independently optionally substituted with one or more R. In some embodiments of a compound disclosed herein, each R^{a} is independently C₁-C₆alkyl or C₁-C₆haloalkyl, wherein each alkyl is independently optionally substituted with one or more R. In some embodiments of a compound disclosed herein, each R^{a} is independently C₁-C₆alkyl or C₁-C₆haloalkyl. In some embodiments of a compound disclosed herein, each R^{a} is independently C₁-C₆alkyl. In some embodiments of a compound disclosed herein, each R^{a} is independently C₁-C₆haloalkyl.

In some embodiments of a compound disclosed herein, each R^{b} is independently hydrogen, C₁-C₆alkyl, C₁-C₆haloalkyl, C₁-C₆hydroxyalkyl, C₁-C₆aminoalkyl, C₁-C₆heteroalkyl, -L-cycloalkyl, or -L-heterocycloalkyl, wherein each alkyl, cycloalkyl, and heterocycloalkyl is independently optionally substituted with one or more R. In some embodiments of a compound disclosed herein, each R^{b} is independently hydrogen, C₁-C₆alkyl, C₁-C₆haloalkyl, -L-cycloalkyl, or -L-heterocycloalkyl, wherein each alkyl, cycloalkyl, and heterocycloalkyl is independently optionally substituted with one or more R. In some embodiments of a compound disclosed herein, each R^{b} is independently hydrogen, C₁-C₆alkyl, C₁-C₆haloalkyl, C₁-C₆hydroxyalkyl, C₁-C₆aminoalkyl, or C₁-C₆heteroalkyl, wherein each alkyl is independently optionally substituted with one or more R. In some embodiments of a compound disclosed herein, each R^{b} is independently hydrogen, C₁-C₆alkyl or C₁-C₆haloalkyl, wherein each alkyl is independently optionally substituted with one or more R. In some embodiments of a compound disclosed herein, each R^{b} is independently hydrogen, C₁-C₆alkyl, or C₁-C₆haloalkyl. In some embodiments of a compound disclosed herein, each R^{b} is independently hydrogen or C₁-C₆alkyl. In some embodiments of a compound disclosed herein, each R^{b} is independently hydrogen or C₁-C₆haloalkyl. In some embodiments of a compound disclosed herein, each R^{b} is hydrogen. In some embodiments of a compound disclosed herein, each R^{b} is independently C₁-C₆alkyl. In some embodiments of a compound disclosed herein, each R^{b} is independently C₁-C₆haloalkyl.

In some embodiments of a compound disclosed herein, each R^{b} is independently hydrogen, C₁-C₆alkyl, C₁-C₆haloalkyl, C₁-C₆hydroxyalkyl, C₁-C₆aminoalkyl, C₁-C₆heteroalkyl, -L-cycloalkyl, or -L-heterocycloalkyl, wherein each alkyl, cycloalkyl, and heterocycloalkyl is independently optionally substituted with one or more R. In some embodiments of a compound disclosed herein, R^{c} and R^{d} are each independently hydrogen, C₁-C₆alkyl, C₁-C₆haloalkyl, -L-cycloalkyl, or -L-heterocycloalkyl, wherein each alkyl, cycloalkyl, and heterocycloalkyl is independently optionally substituted with one or more R. In some embodiments of a compound disclosed herein, R^{c} and R^{d} are each independently hydrogen, C₁-C₆alkyl, C₁-C₆haloalkyl, C₁-C₆hydroxyalkyl, C₁-C₆aminoalkyl, or C₁-C₆heteroalkyl, wherein each alkyl is independently optionally substituted with one or more R. In some embodiments of a compound disclosed herein, R^{c} and R^{d} are each independently hydrogen, C₁-C₆alkyl or C₁-C₆haloalkyl, wherein each alkyl is independently optionally substituted with one or more R. In some embodiments of a compound disclosed herein, R^{c} and R^{d} are each independently hydrogen, C₁-C₆alkyl, or C₁-C₆haloalkyl. In some embodiments of a compound disclosed herein, R^{c} and R^{d} are each independently hydrogen or C₁-C₆alkyl. In some embodiments of a compound disclosed herein, R^{c} and R^{d} are each independently hydrogen or C₁-C₆haloalkyl. In some embodiments of a compound disclosed herein, R^{c} and R^{d} are each hydrogen. In some embodiments of a compound disclosed herein, R^{c} and R^{d} are each independently C₁-C₆alkyl. In some embodiments of a compound disclosed herein, R^{c} and R^{d} are each independently C₁-C₆haloalkyl.

In some embodiments of a compound disclosed herein, R^{c} and R^{d} are taken together with the atom to which they are attached to form a heterocycloalkyl optionally substituted with one or more R.

In some embodiments of a compound disclosed herein, L is absent or C₁-C₃alkylene. In some embodiments of a compound disclosed herein, L is absent. In some embodiments of a compound disclosed herein, L is C₁-C₃alkylene. In some embodiments of a compound disclosed herein, L is —CH₂—. In some embodiments of a compound disclosed herein, L is —CH₂CH₂—. In some embodiments of a compound disclosed herein, L is —CH₂CH₂CH₂—.

In some embodiments of a compound disclosed herein, each R is independently halogen, —CN, —OH, —SF₅, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₆aminoalkyl, C₁-C₆heteroalkyl, C₃-C₆cycloalkyl, or 3- to 6-membered heterocycloalkyl. In some embodiments of a compound disclosed herein, each R is independently halogen, —CN, —OH, —SF₅, —NH₂, —NHC₁-C₃alkyl, —N(C₁-C₃alkyl)₂, C₁-C₃alkyl, C₁-C₆alkoxy, C₁-C₃haloalkyl, C₁-C₃haloalkoxy, C₁-C₃hydroxyalkyl, C₁-C₃aminoalkyl, C₁-C₃heteroalkyl, C₃-C₆cycloalkyl, or 3- to 6-membered heterocycloalkyl. In some embodiments of a compound disclosed herein, each R is independently halogen, —CN, —OH, —SF₅, —NH₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, C₃-C₆cycloalkyl, or 3- to 6-membered heterocycloalkyl. In some embodiments of a compound disclosed herein, each R is independently halogen, —CN, —OH, —NH₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, C₃-C₆cycloalkyl, or 3- to 6-membered heterocycloalkyl. In some embodiments of a compound disclosed herein, each R is independently halogen, —CN, —OH, —NH₂, C₁-C₃alkyl, C₁-C₃alkoxy, C₁-C₃haloalkyl, or C₁-C₃haloalkoxy. In some embodiments of a compound disclosed herein, each R is independently halogen, —CN, —OH, —NH₂, C₁-C₃alkyl, or C₁-C₃haloalkyl. In some embodiments of a compound disclosed herein, each R is independently halogen, —CN, —OH, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, or C₁-C₃haloalkyl. In some embodiments of a compound disclosed herein, each R is independently halogen, —CN, —OH, —C(═O)OH, —C(═O)OC₁-C₃alkyl, C₁-C₃alkyl, or C₁-C₃haloalkyl. In some embodiments of a compound disclosed herein, each R is independently halogen, —CN, —OH, —C(═O)OH, —C(═O)OC₁-C₃alkyl, or C₁-C₃alkyl.

In some embodiments, disclosed herein is a radiopharmaceutical conjugate comprising a covalently bound radioisotope. In some embodiments of Formula (AC), (AC′), (BC), (BC′), (CC), or (CC′), R³⁰ comprises the covalently bound radioisotope. In some embodiments of Formula (AC), (BC), or (CC), TL₁ comprises the covalently bound radioisotope. In some embodiments of Formula (AC), (BC), or (CC), TL₂ comprises the covalently bound radioisotope. In some embodiments of Formula (AC), (BC), or (CC), L^{A} comprises the covalently bound radioisotope. In some embodiments of Formula (AC′), (BC′), or (CC′), one or both of the TL₁ groups comprise the covalently bound radioisotope. In some embodiments of Formula (AC′), (BC′), or (CC′), one or both of the TL₂ groups comprise the covalently bound radioisotope. In some embodiments of Formula (AC′), (BC′), or (CC′), L^{B} comprises the covalently bound radioisotope. In some embodiments of Formula (BC), (BC′), (CC), or (CC′), X comprises the covalently bound radioisotope.

In some embodiments of Formula (AC), (AC′), (BC), (BC′), (CC), or (CC′), R³⁰ is an albumin binding moiety.

In some embodiments of Formula (AC), (AC′), (BC), (BC′), (CC), or (CC′), R³⁰ is a payload moiety. In some embodiments, the payload moiety comprises a payload and a linker connecting the payload to group L^{A} or L^{B}. In some embodiments, the payload moiety is a payload having cytotoxic, therapeutic, or diagnostic properties. In some embodiments, the payload moiety comprises a radioisotope. In some embodiments, the payload moiety comprises a covalently bound radioisotope. In some embodiments, the payload moiety comprises a chelator. In some embodiments, the payload moiety comprises a chelator and a radioisotope bound to the chelator. In some embodiments, payload is a toxin. In some embodiments, payload is a therapeutic agent. In some embodiments, the payload moiety comprises a cleavable linker and a toxin (e.g., an auristatin, such as auristatin E or a derivative thereof). Exemplary cleavable linkers can be found in, e.g., U.S. Pat. No. 9,962,454B².

In some embodiments, the toxin is a taxane, a topoisomerase inhibitor, a vinca alkaloid, or the like.

In some embodiments, the toxin can be selected from, for example, DNA minor groove binders (e.g., enediynes and lexitropsins, a CBI compound; see also U.S. Pat. No. 6,130,237), duocarmycins, taxanes (e.g., paclitaxel and docetaxel), puromycins, and vinca alkaloids. Other cytotoxic agents include, for example, CC-1065, SN-38, topotecan, morpholino-doxorubicin, rhizoxin, cyanomorpholino-doxorubicin, echinomycin, combretastatin, netropsin, epothilone A and B, estramustine, cryptophysins, cemadotin, maytansinoids, discodermolide, eleutherobin, and mitoxantrone.

In some embodiments, the toxin is an anti-tubulin agent. Examples of anti-tubulin agents include, auristatins, taxanes (e.g., Taxol® (paclitaxel), Taxotere® (docetaxel)), T67 (Tularik) and vinca alkyloids (e.g., vincristine, vinblastine, vindesine, and vinorelbine). Examples of anti-tubulin agents further include, for example, baccatin derivatives, taxane analogs (e.g., epothilone A and B), nocodazole, colchicine and colcimid, estramustine, cryptophycins, cemadotin, maytansinoids, combretastatins, discodermolide, and eleutherobin.

In some embodiments, the toxin is a maytansinoid, another group of anti-tubulin agents. For example, in some embodiments, the maytansinoid is maytansine or DM-1.

In some embodiments, the toxin is a dolastatin. In some embodiments, the toxin is of the auristatin class. In some embodiments, the toxin is MMAE. In some embodiments, the toxin is AFP.

In some embodiments of Formula (AC), (AC′), (BC), (BC′), (CC), or (CC′), R³⁰ is hydrogen, halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted (e.g., with one or more R). In some embodiments of Formula (AC), (AC′), (BC), (BC′), (CC), or (CC′), R³⁰ is hydrogen.

Any combination of the groups described above for the various variables is contemplated herein. Throughout the specification, groups and substituents thereof are chosen by one skilled in the field to provide stable moieties and compounds.

### Metal Chelator

In one aspect, described herein are conjugates that comprise a metal chelator, e.g., as represented by CL in the formulas described herein.

In some embodiments, the metal chelator binds with a radionuclide. In some embodiments, the conjugate described herein comprises two or more independent metal chelators, e.g., 2, 3, 4, 5, or more metal chelators. In some embodiments, the conjugate described herein comprises two metal chelators, which can be the same or different. In some embodiments, the conjugate described herein comprises two or more metal chelators. In some embodiments, the conjugate comprises two radionuclides bound to the metal chelators.

In some embodiments, the metal chelator is capable of binding a radioactive atom. The binding can be direct, e.g., the metal chelator can make hydrogen bonds or electrostatic interactions with the radioactive atom. The binding can also be indirect, e.g., the metal chelator binds to a molecule that comprises a radioactive atom. In some embodiments, the metal chelator comprises, or is, a macrocycle. In some embodiments, the metal chelator comprises, or is, 2,2′,2″,2′″-(1,4,7,10-Tetraazacyclododecane-1,4,7,10-tetrayl)tetraacetic acid (DOTA) or 1,4,7-triazacyclononane-1,4,7-triacetic acid (NOTA). In some embodiments, the metal chelator comprises a macrocycle, e.g., a macrocycle comprising an oxygen atom and/or a nitrogen atom, DOTA, NOTA, one or more amines, one or more ethers, one or more carboxylic acids, EDTA, DTPA, TETA, DO3A, PCTA, or desferrioxamine.

In some embodiments, the metal chelator comprises a plurality of amines. In some embodiments, the metal chelator includes 4 or more nitrogen atoms, 4 or more carboxylic acid groups, or a combination thereof. In some embodiments, the metal chelator does not comprise S. In some embodiments, the metal chelator comprises a ring. In some embodiments, the ring comprises an oxygen atom and/or a nitrogen atom. In some embodiments, the metal chelator is a ring that includes 3 or more nitrogen atom, 3 or more carboxylic acid groups, or a combination thereof. In some embodiments, the metal chelator is polydentate.

In some embodiments, the metal chelator (or CL) described herein is DOTA, DOTA-GA, pBn-DOTA, pBn-SCN-DOTA, NH2-DOTA, NH2-DOTA-GA, p-NCS-Bn-DOTA-GA, p-NH2-Bn-oxo-DO3A, p-SCN-Bn-oxo-DO3A, NOTA, NODA-GA, NH2-NODA-GA, p-NCS-Bn-NODA-GA, p-NH2-Bn-NOTA, p-SCN-Bn-NOTA, NCS-MP-NODA, NH2-MPAA-NODA, PCTA, p-NH2-Bn-PCTA, p-SCN-Bn-PCTA, p-SCN-Bn-HEHA, H2-MACROPA-NCS, H1-MACROPA, H2-MACROPA-NH2, H4-OCTAPA, tetra-(S, S, S, S)-Me-DOTA, tetra-(S, S, S, S)-Et-DOTA, tetra-(S, S, S, S)-iBu-DOTA, or maleimide-nBu-DOTA.

In some embodiments, the metal chelator (or CL) described herein comprises a cyclic chelating agent. Exemplary cyclic chelating agents include, but are not limited to, AAZTA, BAT, BAT-TM, Crown, Cyclen, DO2A, CB-DO2A, DO3A, H3HP-DO3A, Oxo-DO3A, p-NH2-Bn-Oxo-DO3A, DOTA, DOTA-3py, DOTA-PA, DOTA-GA, DOTA-4AMP, DOTA-2py, DOTA-1py, p-SCN-Bn-DOTA, CHX-A″-EDTA, MeO-DOTA-NCS EDTA, DOTAMAP, DOTAGA, DOTAGA-anhydride, DOTMA, DOTASA, DOTAM, DOTP, CB-Cyclam, TE2A, CB-TE2A, CB-TE2P, DM-TE2A, MM-TE2A, NOTA, NOTP, HEHA, HEHA-NCS, p-SCN-Bn-HEHA, DTPA, CHX-A″-DTPA, p-NH₂-Bn-CHX-A″-DTPA, p-SCN-DTPA, p-SCN-Bz-Mx-DTPA, 1B4M-DTPA, p-SCN-Bn1B-DTPA, p-SCN-Bn-1B4M-DTPA, p-SCN-Bn-CHX-A″-DTPA, PEPA, p-SCN-Bn-PEPA, TETPA, DOTPA, DOTMP, DOTPM, t-Bu-calix[4]arene-tetracarboxylic acid, macropa, macropa-NCS, macropid, H₃L¹, H₃L⁴, H₂azapa, H₅decapa, bispa², H₄pypa, H₄octapa, H₄CHXoctapa, p-SCN-Bn-Hoctapa, p-SCN-Bn-H₄octapa, TTHA, p-NO₂-Bn-neunpa, H₄octox, H₂macropa, H₂bispa², H₄phospa, H₆phospa, p-SCN-Bn-H₆phospa, TETA, p-NO₂-Bn-TETA, TRAP, TPA, HBED, SHBED, HBED-CC, (HBED-CC) TFP, DMSA, DMPS, DHLA, lipoic acid, TGA, BAL, Bis-thioseminarabazones, p-SCN-NOTA, nNOTA, NODAGA, CB-TE1AIP, 3P-C-NETA-NCS, 3p-C-DEPA, 3P-C-DEPA-NCS, TCMC, PCTA, NODIA-Me, TACN, pycup1A¹B, pycup2A, THP, DEDPA, H₂DEDPA, p-SCN-Bn-H₂DEDPA, p-SCN-Bn-TCMC, motexafin, NTA, NOC, 3p-C-NETA, p-NH₂-Bn-TE3A, SarAr, DiAmSar, SarAr-NCS, AmBaSar, BaBaSar, TACN-TM, CP256, C-NE3TA, C-NE3TA-NCS, NODASA, NETA-monoamide, C-NETA, NOPO, BPCA, p-SCN-Bn-DFO, DFO-ChX-Mal, DFO, DFO-IAC, DFO-BAC, DiP-LICAM, EC, SBAD, BAPEN, TACHPYR, NEC-SP, L^{py}, L1, L2, L3, and EuK-106. In some embodiments, the metal chelator is DOTA, TRITA, TETA, DOTA-MA, DO3A-HP, DOTMA, DOTA-pNB, DOTP, DOTMP, DOTEP, DOTMPE, F-DOTPME, DOTPP, DOTBzP, DOTA-monoamide, p-NCS-DOTA, p-NCS-PADOTA, BAT, DO3TMP-Monoamide, p-NCS-TRITA, NOTA, or CHX-A″-DTPA. In some embodiments, the metal chelator described herein comprises an acyclic chelating agent. Exemplary acyclic chelating agents include, but are not limited to, DTA, CyEDTA, EDTMP, DTPMP, DTPA, CyDTPA, Cy2DTPA, DTPA-MA, DTPA-BA, and BOPA. In some embodiments, the metal chelator described herein comprises DOTA, DOTP, DOTMA, DOTAM, DTPA, NTA, EDTA, DO3A, DO2A, NOC, NOTA, TETA, TACN, DiAmSar, CB-Cyclam, CB-TE2A, DOTA-4AMP, or NOTP. In some embodiments, the metal chelator described herein comprises H₄pypa, H₄octox, H₄octapa, p-NO₂-Bn-neunpa, p-SCN-Bn-H₄neunpa, TTHA, ¹Bu₄pypa-C7-NHS, H₄neunpa, H₂macropa, HP-DO3A, BT-DO3A, DO3A-Nprop, DO3AP, DO2A²P, DOA3P, DOTP, DOTPMB, DOTAMAE, DOTAMAP, DO3AM^{Bu}, DOTMA, TCE-DOTA, DEPA, PCTA, p-NO₂-Bn-PCTA, p-NO₂-Bn-DOTA, symPC2APA, symPCA2PA, asymPC2APA, asymPCA2PA, TRAP, AAZTA, DATA^{m}, THP, HEHA, or HBED.

In some embodiments, the metal chelator (or CL) is DO3A. In some embodiments, the metal chelator is PEPA. In some embodiments, the metal chelator is EDTA. In some embodiments, the metal chelator is CHX-A”-DTPA. In some embodiments, the metal chelator is HEHA. In some embodiments, the metal chelator is DOTMP. In some embodiments, the metal chelator is t-Bu-calix[4]arene-tetracarboxylic acid. In some embodiments, the metal chelator is macropa. In some embodiments, the metal chelator is macropa-NCS. In some embodiments, the metal chelator is H4pypa. In some embodiments, the metal chelator is H₄octapa. In some embodiments, the metal chelator is H₄CHXoctapa. In some embodiments, the metal chelator is DOTP. In some embodiments, the metal chelator is crown.

Exemplary metal chelators are further described in WO2012/174136; US20130183235A1; US20120219495A1; Ramogidaand et al., EJNMMI radiopharm. chem. 4, 21 (2019); Thiele et al., Cancer Biotherapy and Radiopharmaceuticals 2018; Li et al., Bioconjugate Chem. 2019, 30, 5, 1539-1553; and Baranyai et al., Eur. J. Inorg. Chem. 36-56 (2020), Dai et al., Nature Communications (2018) 9:857, each of which is incorporated by reference in its entirety.

In some embodiments, the metal chelator (or CL) is DOTA. In some embodiments, the metal chelator is a chiral derivative of DOTA. In some embodiments, the metal chelator is 2,2′,2″,2′″-((2S,5S,8S,11S)-2,5,8,11-tetramethyl-1,4,7,10-tetraazacyclododecane-1,4,7,10-tetrayl)tetraacetic acid.

In some embodiments, the metal chelator (or CL) is

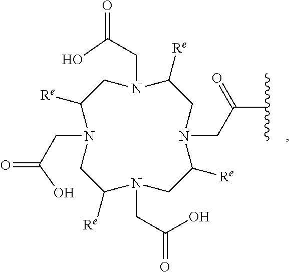

wherein each R^{c} is independently selected from hydrogen, alkyl, haloalkyl, hydroxyalkyl, aminoalkyl, heteroalkyl, alkenyl, alkynyl, cycloalkyl, heterocycloalkyl, aryl, heteroaryl, alkylcycloalkyl, alkylheterocycloalkyl, alkylaryl, alkylheteroaryl, or an amino acid side chain. In some embodiments, the metal chelator is

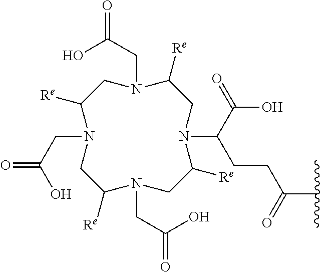

wherein each R^{e} is independently selected from hydrogen, alkyl, haloalkyl, hydroxyalkyl, aminoalkyl, heteroalkyl, alkenyl, alkynyl, cycloalkyl, heterocycloalkyl, aryl, heteroaryl, alkylcycloalkyl, alkylheterocycloalkyl, alkylaryl, alkylheteroaryl, or an amino acid side chain.

In some embodiments, the metal chelator (or CL) is

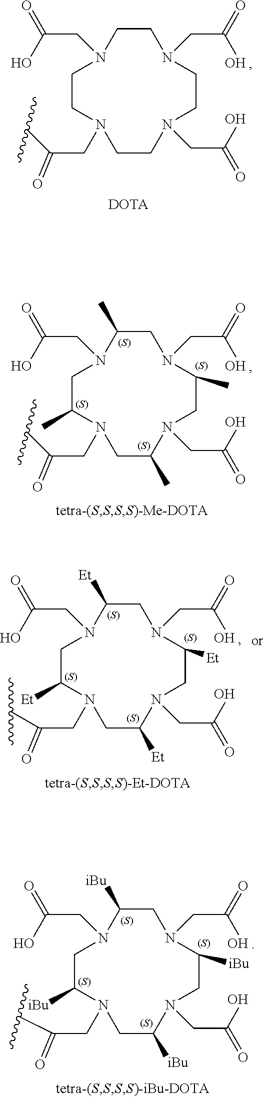

In some embodiments, the metal chelator is

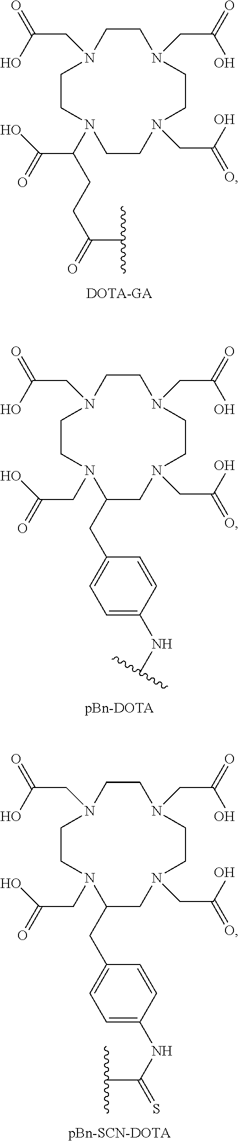 [SMILES: *C(=O)CCC(C(=O)O)N1CCN(CC(=O)O)CCN(CC(=O)O)CCN(CC(=O)O)CC1 ; *Nc1ccc(CC2CN(CC(=O)O)CCN(CC(=O)O)CCN(CC(=O)O)CCN2CC(=O)O)cc1 ; *C(=S)Nc1ccc(CC2CN(CC(=O)O)CCN(CC(=O)O)CCN(CC(=O)O)CCN2CC(=O)O)cc1]

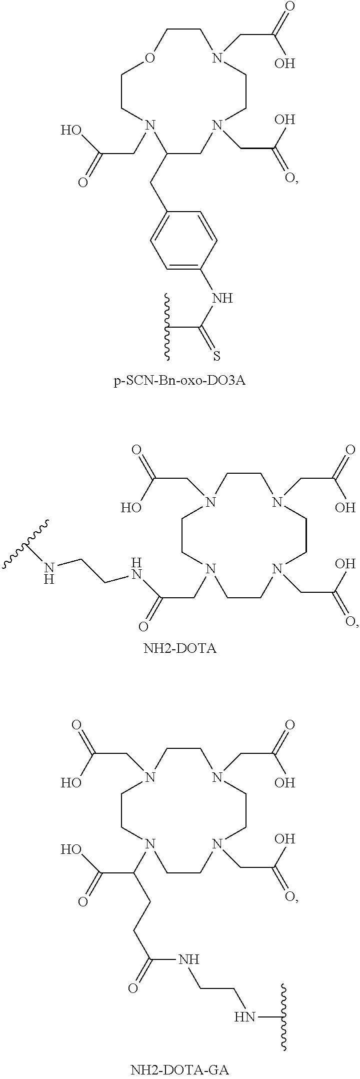

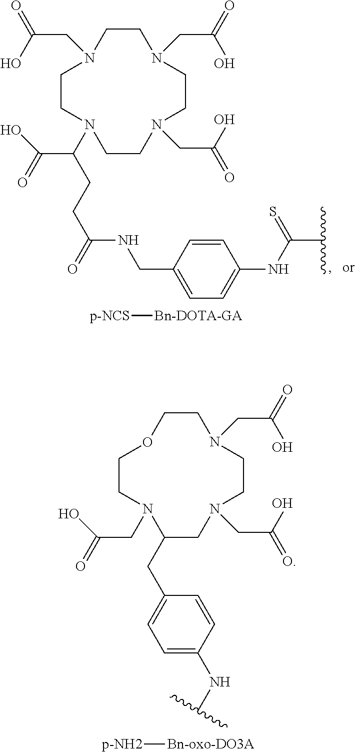

In some embodiments, the metal chelator is

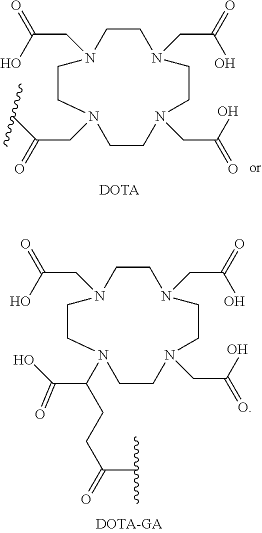

In some embodiments, the metal chelator is

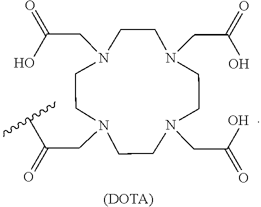

In some embodiments, the metal chelator is

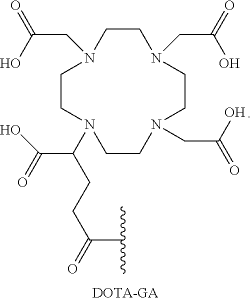 [SMILES: *C(=O)CCC(C(=O)O)N1CCN(CC(=O)O)CCN(CC(=O)O)CCN(CC(=O)O)CC1]

In some embodiments, the metal chelator is

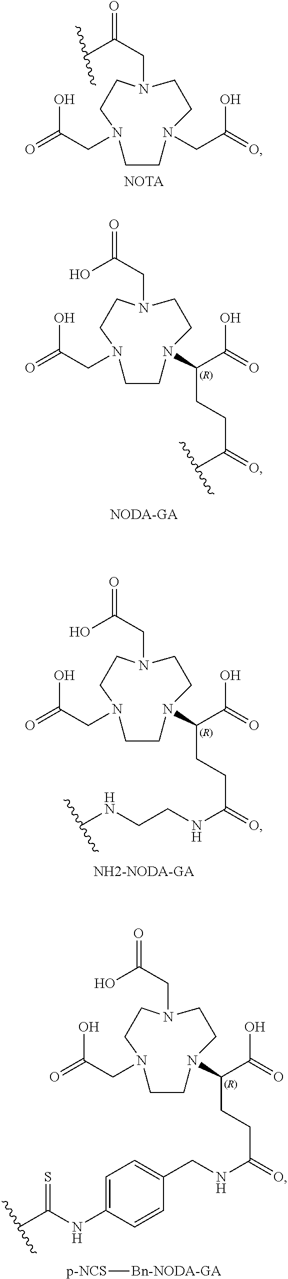

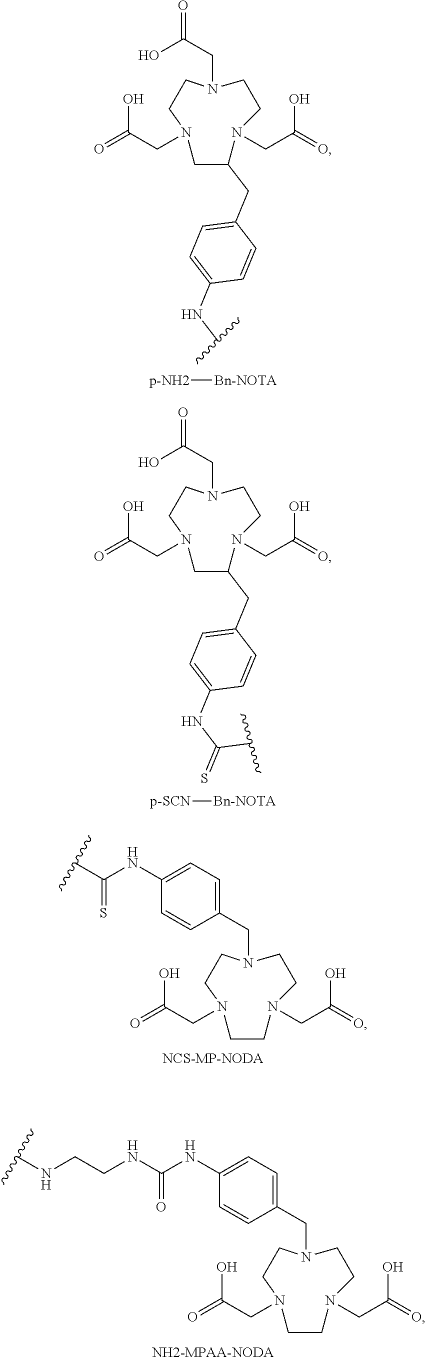

or In some embodiments, the metal chelator is

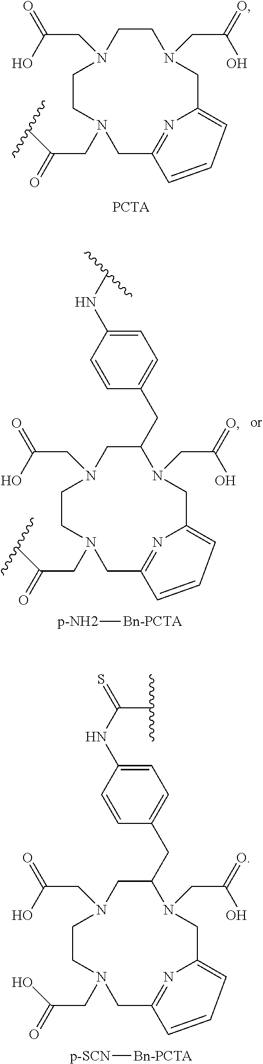

In some embodiments, the metal chelator (or CL) is

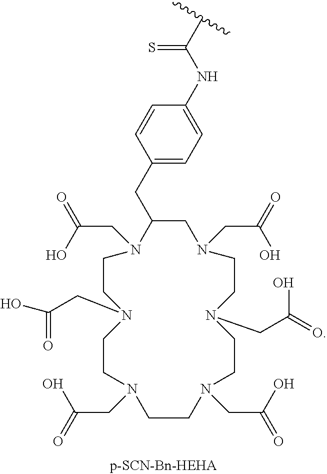 [SMILES: *C(=S)Nc1ccc(CC2CN(CC(=O)O)CCN(CC(=O)O)CCN(CC(=O)O)CCN(CC(=O)O)CCN(CC(=O)O)CCN2CC(=O)O)cc1]

In some embodiments, the metal chelator is

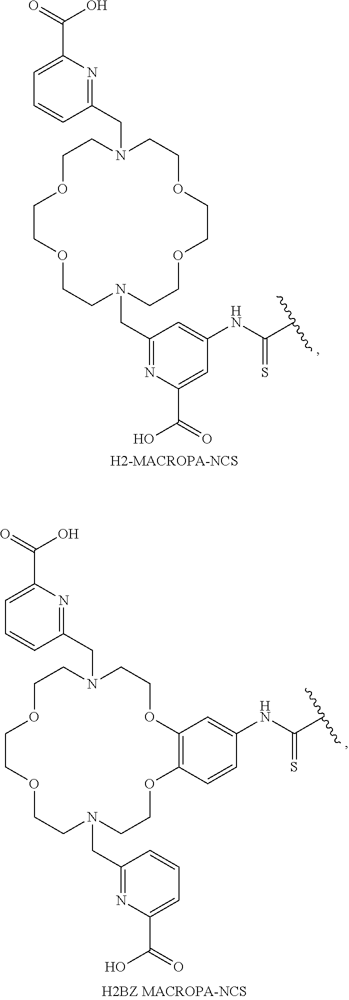

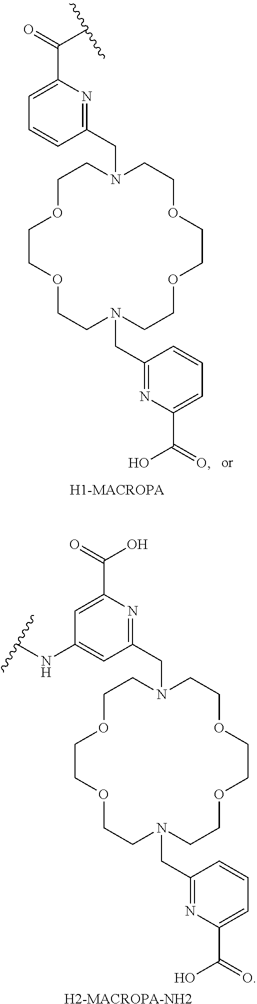

Exemplary metal chelators are also illustrated in FIG. 1 to FIG. 19.

In some embodiments the conjugate disclosed herein, or a pharmaceutically acceptable salt or stereoisomer thereof, is one of the compounds in Table 1.

TABLE 1

| Compound | Structure |
|---|---|
| 100 | 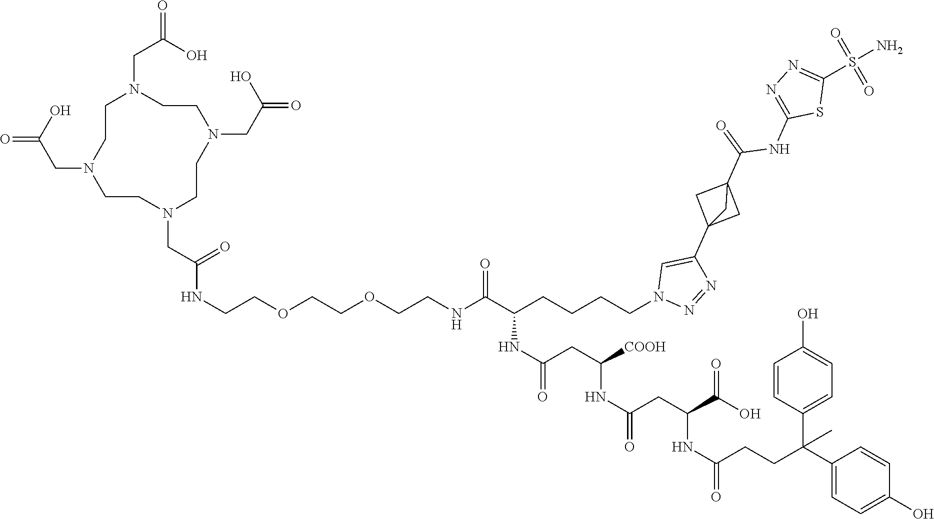 [SMILES: CC(CCC(=O)N[C@@H](CC(=O)N[C@@H](CC(=O)N[C@@H](CCCCn1cc(C23CC(C(=O)Nc4nnc(S(N)(=O)=O)s4)(C2)C3)nn1)C(=O)NCCOCCOCCNC(=O)CN1CCN(CC(=O)O)CCN(CC(=O)O)CCN(CC(=O)O)CC1)C(=O)O)C(=O)O)(c1ccc(O)cc1)c1ccc(O)cc1] |
| 100.1 | 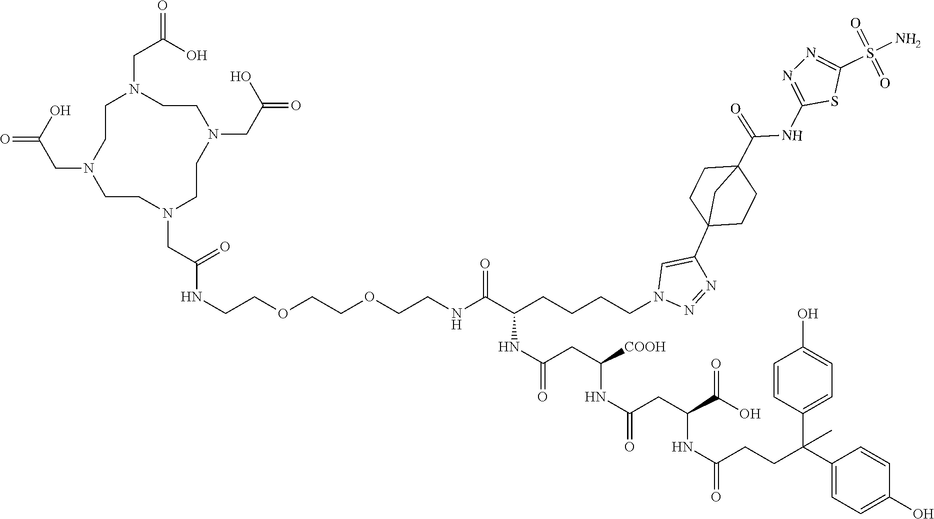 [SMILES: CC(CCC(=O)N[C@@H](CC(=O)N[C@@H](CC(=O)N[C@@H](CCCCn1cc(C23CCC(C(=O)Nc4nnc(S(N)(=O)=O)s4)(CC2)C3)nn1)C(=O)NCCOCCOCCNC(=O)CN1CCN(CC(=O)O)CCN(CC(=O)O)CCN(CC(=O)O)CC1)C(=O)O)C(=O)O)(c1ccc(O)cc1)c1ccc(O)cc1] |
| 100.2 | 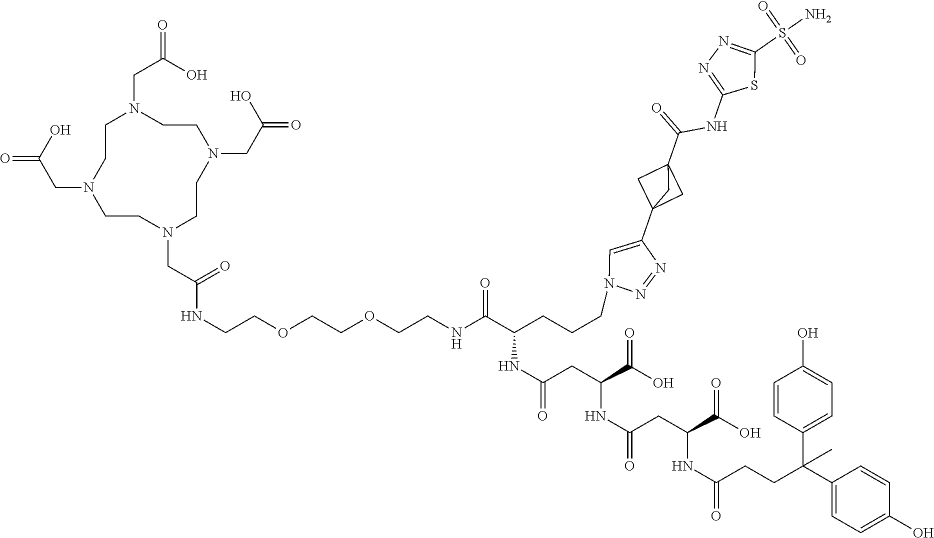 [SMILES: CC(CCC(=O)N[C@@H](CC(=O)N[C@@H](CC(=O)N[C@@H](CCCn1cc(C23CC(C(=O)Nc4nnc(S(N)(=O)=O)s4)(C2)C3)nn1)C(=O)NCCOCCOCCNC(=O)CN1CCN(CC(=O)O)CCN(CC(=O)O)CCN(CC(=O)O)CC1)C(=O)O)C(=O)O)(c1ccc(O)cc1)c1ccc(O)cc1] |
| 100.3 | 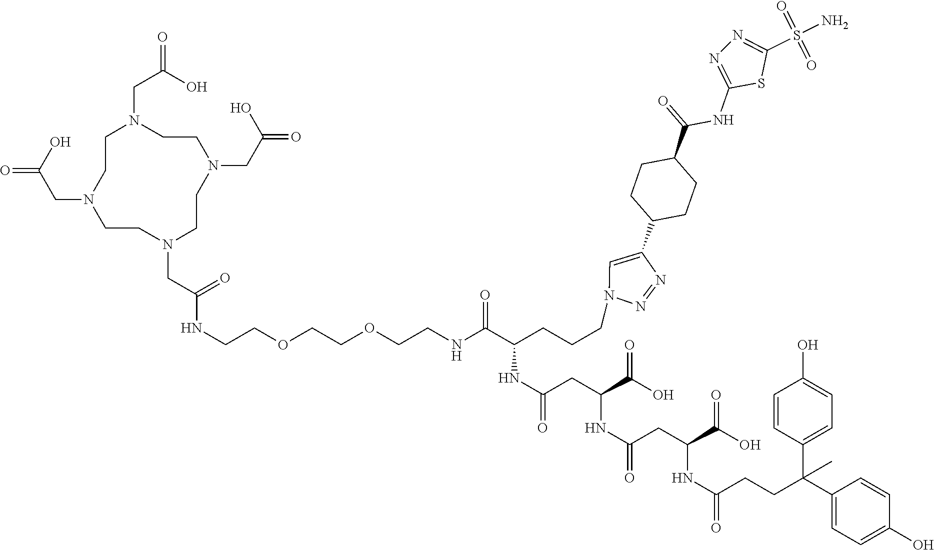 |
| 100.4 | 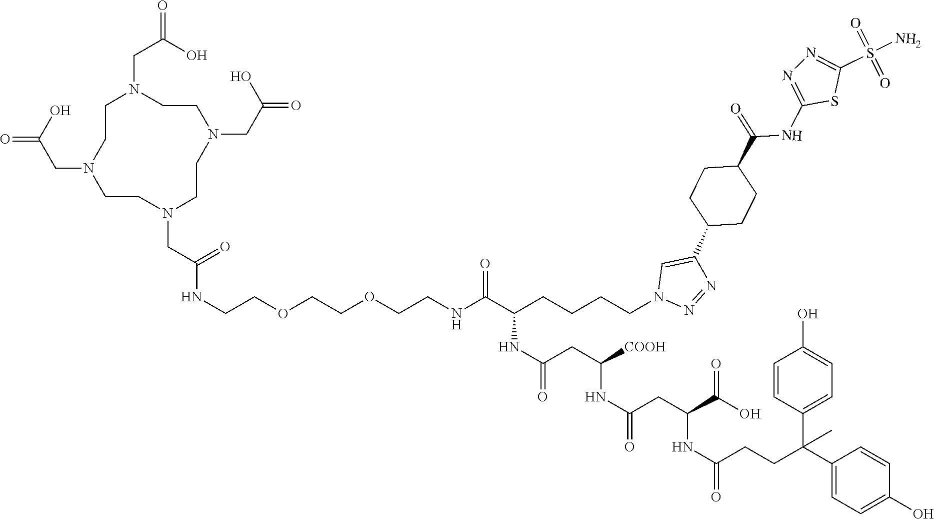 [SMILES: CC(CCC(=O)N[C@@H](CC(=O)N[C@@H](CC(=O)N[C@@H](CCCCn1cc([C@H]2CC[C@H](C(=O)Nc3nnc(S(N)(=O)=O)s3)CC2)nn1)C(=O)NCCOCCOCCNC(=O)CN1CCN(CC(=O)O)CCN(CC(=O)O)CCN(CC(=O)O)CC1)C(=O)O)C(=O)O)(c1ccc(O)cc1)c1ccc(O)cc1] |
| 100.5 | 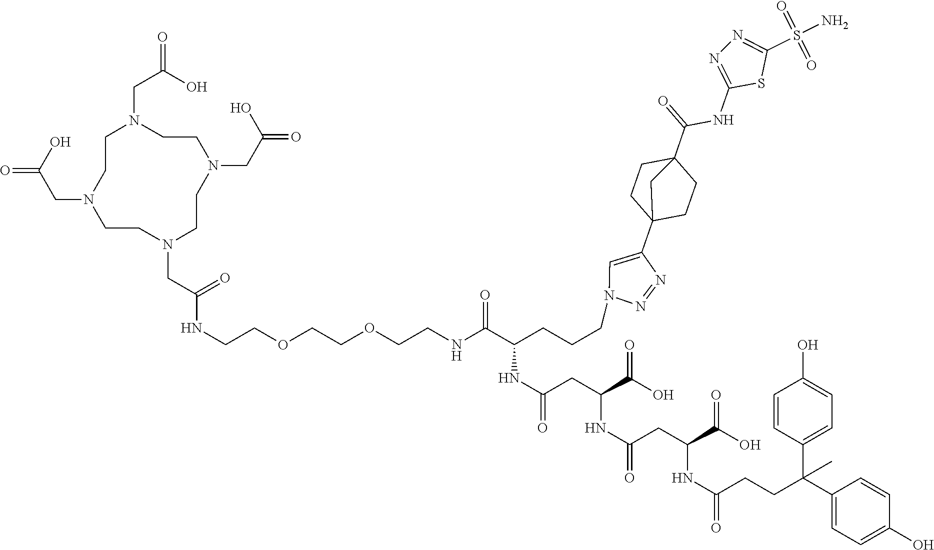 [SMILES: CC(CCC(=O)N[C@@H](CC(=O)N[C@@H](CC(=O)N[C@@H](CCCn1cc(C23CCC(C(=O)Nc4nnc(S(N)(=O)=O)s4)(CC2)C3)nn1)C(=O)NCCOCCOCCNC(=O)CN1CCN(CC(=O)O)CCN(CC(=O)O)CCN(CC(=O)O)CC1)C(=O)O)C(=O)O)(c1ccc(O)cc1)c1ccc(O)cc1] |
| 100.6 | 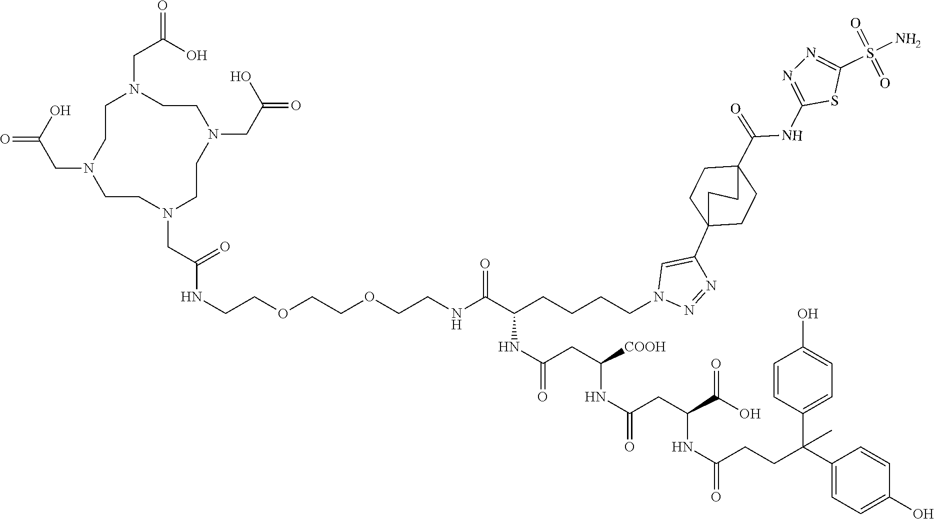 [SMILES: CC(CCC(=O)N[C@@H](CC(=O)N[C@@H](CC(=O)N[C@@H](CCCCn1cc(C23CCC(C(=O)Nc4nnc(S(N)(=O)=O)s4)(CC2)CC3)nn1)C(=O)NCCOCCOCCNC(=O)CN1CCN(CC(=O)O)CCN(CC(=O)O)CCN(CC(=O)O)CC1)C(=O)O)C(=O)O)(c1ccc(O)cc1)c1ccc(O)cc1] |
| 100.7 | 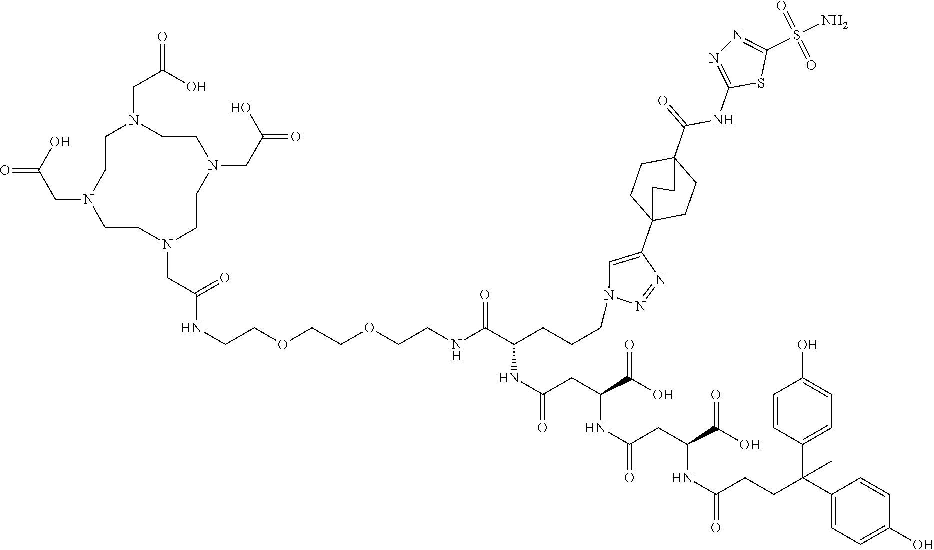 [SMILES: CC(CCC(=O)N[C@@H](CC(=O)N[C@@H](CC(=O)N[C@@H](CCCn1cc(C23CCC(C(=O)Nc4nnc(S(N)(=O)=O)s4)(CC2)CC3)nn1)C(=O)NCCOCCOCCNC(=O)CN1CCN(CC(=O)O)CCN(CC(=O)O)CCN(CC(=O)O)CC1)C(=O)O)C(=O)O)(c1ccc(O)cc1)c1ccc(O)cc1] |
| 100.8 | 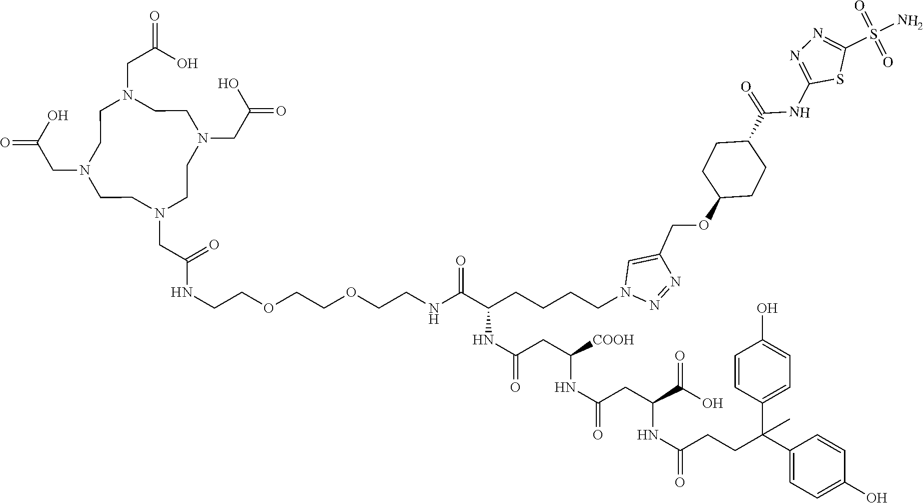 [SMILES: CC(CCC(=O)N[C@@H](CC(=O)N[C@@H](CC(=O)N[C@@H](CCCCn1cc(CO[C@H]2CC[C@H](C(=O)Nc3nnc(S(N)(=O)=O)s3)CC2)nn1)C(=O)NCCOCCOCCNC(=O)CN1CCN(CC(=O)O)CCN(CC(=O)O)CCN(CC(=O)O)CC1)C(=O)O)C(=O)O)(c1ccc(O)cc1)c1ccc(O)cc1] |
| 100.9 | 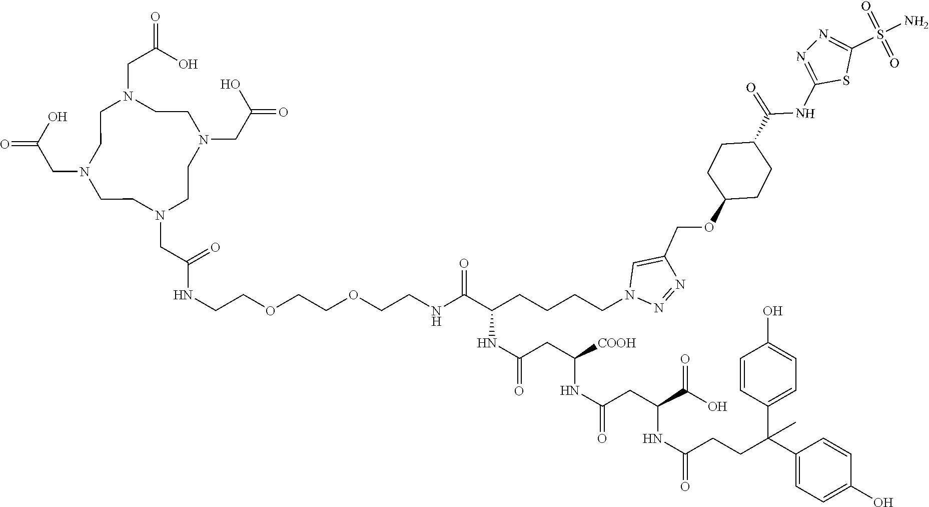 [SMILES: CC(CCC(=O)N[C@@H](CC(=O)N[C@@H](CC(=O)N[C@@H](CCCCn1cc(CO[C@H]2CC[C@H](C(=O)Nc3nnc(S(N)(=O)=O)s3)CC2)nn1)C(=O)NCCOCCOCCNC(=O)CN1CCN(CC(=O)O)CCN(CC(=O)O)CCN(CC(=O)O)CC1)C(=O)O)C(=O)O)(c1ccc(O)cc1)c1ccc(O)cc1] |
| 100.10 | 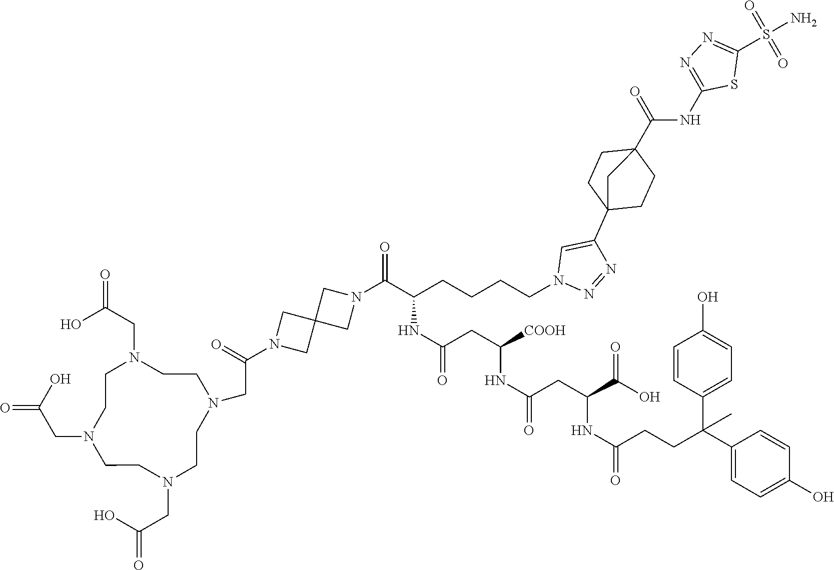 [SMILES: CC(CCC(=O)N[C@@H](CC(=O)N[C@@H](CC(=O)N[C@@H](CCCCn1cc(C23CCC(C(=O)Nc4nnc(S(N)(=O)=O)s4)(CC2)C3)nn1)C(=O)N1CC2(CN(C(=O)CN3CCN(CC(=O)O)CCN(CC(=O)O)CCN(CC(=O)O)CC3)C2)C1)C(=O)O)C(=O)O)(c1ccc(O)cc1)c1ccc(O)cc1] |
| 100.11 | 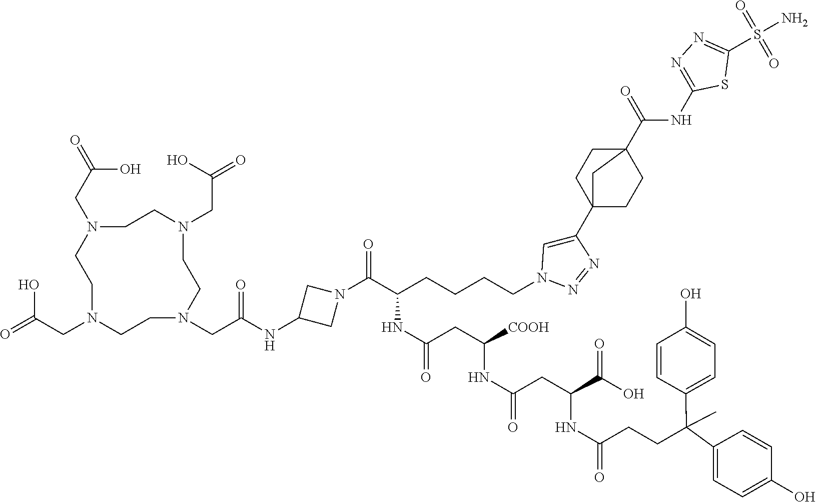 |
| 100.12 | 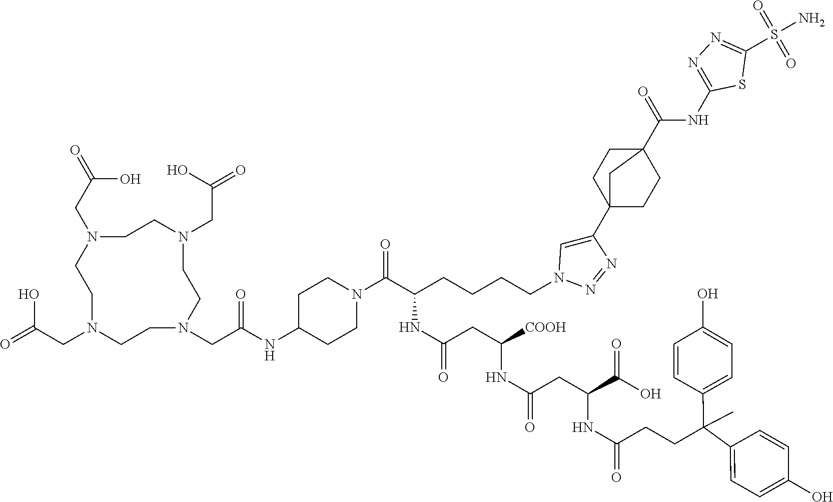 |
| 100.13 | 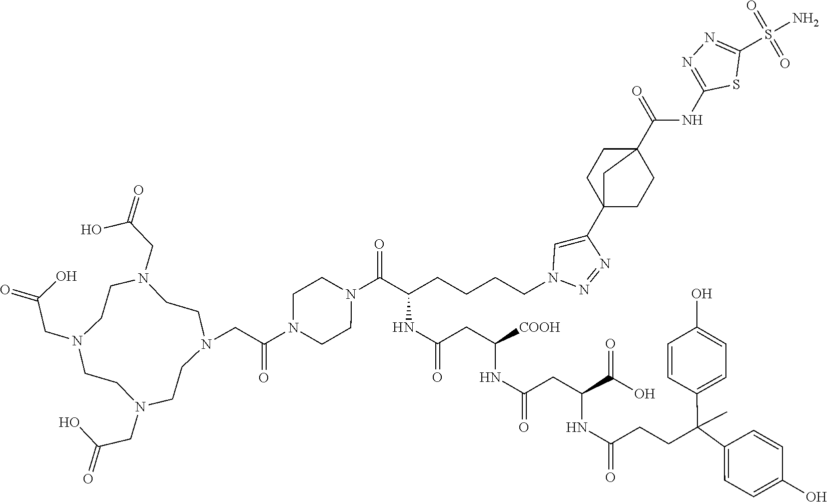 [SMILES: CC(CCC(=O)N[C@@H](CC(=O)N[C@@H](CC(=O)N[C@@H](CCCCn1cc(C23CCC(C(=O)Nc4nnc(S(N)(=O)=O)s4)(CC2)C3)nn1)C(=O)N1CCN(C(=O)CN2CCN(CC(=O)O)CCN(CC(=O)O)CCN(CC(=O)O)CC2)CC1)C(=O)O)C(=O)O)(c1ccc(O)cc1)c1ccc(O)cc1] |
| 100.14 | 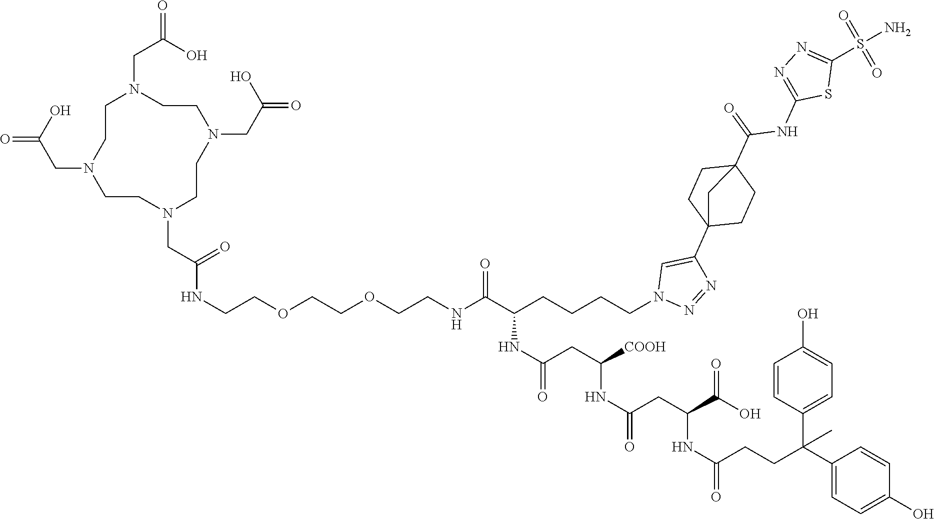 [SMILES: CC(CCC(=O)N[C@@H](CC(=O)N[C@@H](CC(=O)N[C@@H](CCCCn1cc(C23CCC(C(=O)Nc4nnc(S(N)(=O)=O)s4)(CC2)C3)nn1)C(=O)NCCOCCOCCNC(=O)CN1CCN(CC(=O)O)CCN(CC(=O)O)CCN(CC(=O)O)CC1)C(=O)O)C(=O)O)(c1ccc(O)cc1)c1ccc(O)cc1] |
| 100.15 | 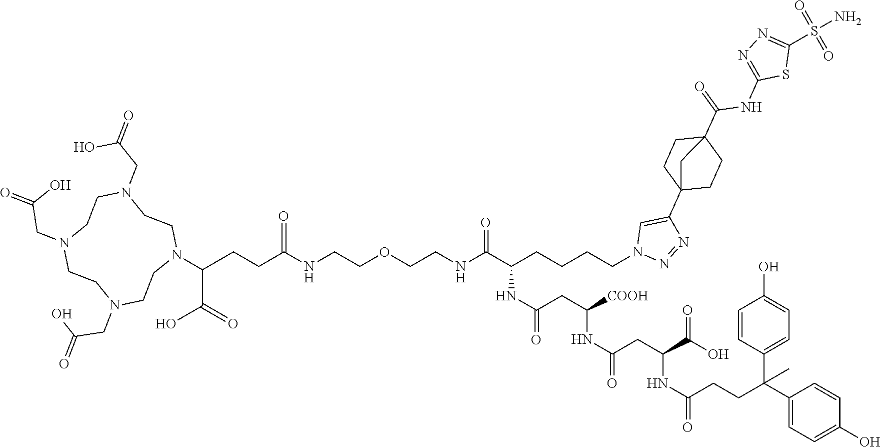 [SMILES: CC(CCC(=O)N[C@@H](CC(=O)N[C@@H](CC(=O)N[C@@H](CCCCn1cc(C23CCC(C(=O)Nc4nnc(S(N)(=O)=O)s4)(CC2)C3)nn1)C(=O)NCCOCCNC(=O)CCC(C(=O)O)N1CCN(CC(=O)O)CCN(CC(=O)O)CCN(CC(=O)O)CC1)C(=O)O)C(=O)O)(c1ccc(O)cc1)c1ccc(O)cc1] |
| 100.16 | 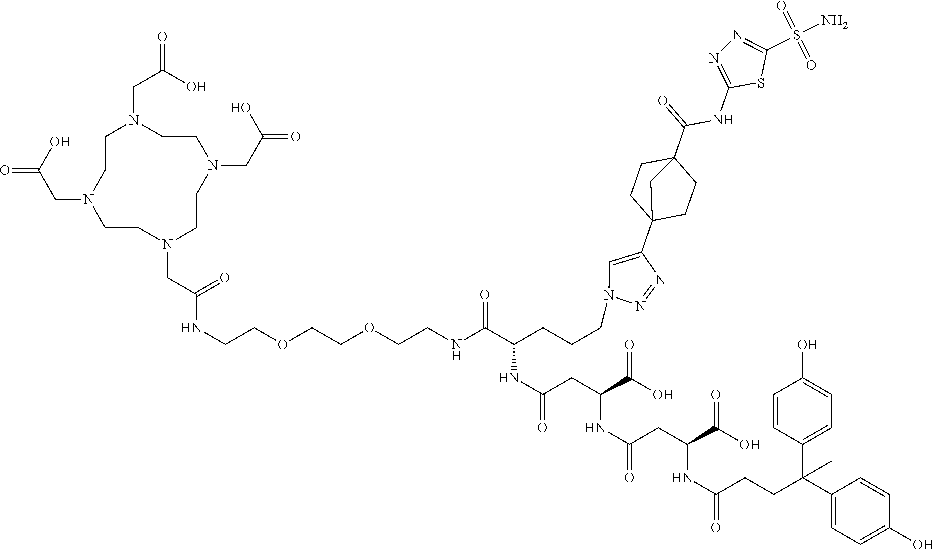 [SMILES: CC(CCC(=O)N[C@@H](CC(=O)N[C@@H](CC(=O)N[C@@H](CCCn1cc(C23CCC(C(=O)Nc4nnc(S(N)(=O)=O)s4)(CC2)C3)nn1)C(=O)NCCOCCOCCNC(=O)CN1CCN(CC(=O)O)CCN(CC(=O)O)CCN(CC(=O)O)CC1)C(=O)O)C(=O)O)(c1ccc(O)cc1)c1ccc(O)cc1] |
| 100.17 | 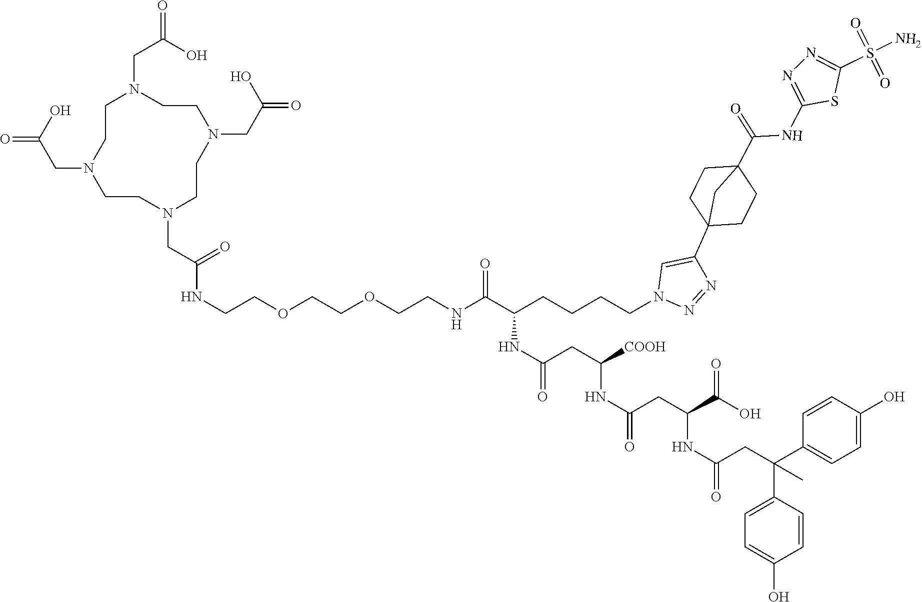 [SMILES: CC(CC(=O)N[C@@H](CC(=O)N[C@@H](CC(=O)N[C@@H](CCCCn1cc(C23CCC(C(=O)Nc4nnc(S(N)(=O)=O)s4)(CC2)C3)nn1)C(=O)NCCOCCOCCNC(=O)CN1CCN(CC(=O)O)CCN(CC(=O)O)CCN(CC(=O)O)CC1)C(=O)O)C(=O)O)(c1ccc(O)cc1)c1ccc(O)cc1] |
| 100.18 | 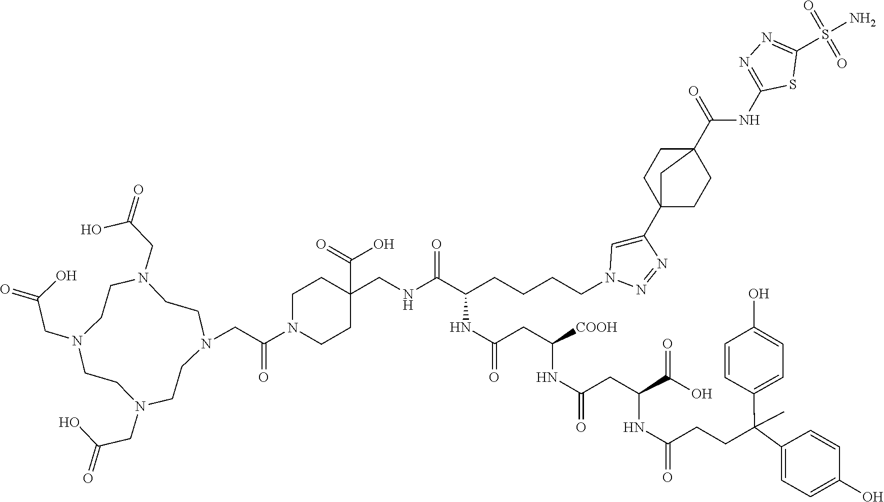 [SMILES: CC(CCC(=O)N[C@@H](CC(=O)N[C@@H](CC(=O)N[C@@H](CCCCn1cc(C23CCC(C(=O)Nc4nnc(S(N)(=O)=O)s4)(CC2)C3)nn1)C(=O)NCC1(C(=O)O)CCN(C(=O)CN2CCN(CC(=O)O)CCN(CC(=O)O)CCN(CC(=O)O)CC2)CC1)C(=O)O)C(=O)O)(c1ccc(O)cc1)c1ccc(O)cc1] |
| 100.19 | 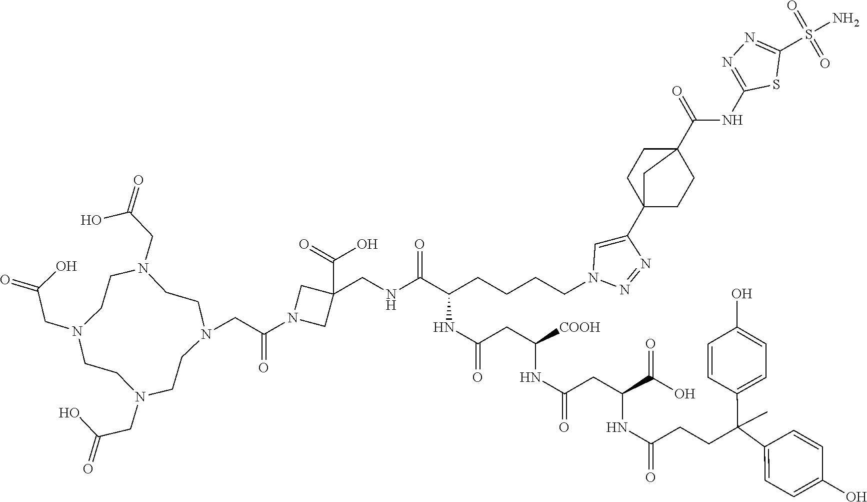 [SMILES: CC(CCC(=O)N[C@@H](CC(=O)N[C@@H](CC(=O)N[C@@H](CCCCn1cc(C23CCC(C(=O)Nc4nnc(S(N)(=O)=O)s4)(CC2)C3)nn1)C(=O)NCC1(C(=O)O)CN(C(=O)CN2CCN(CC(=O)O)CCN(CC(=O)O)CCN(CC(=O)O)CC2)C1)C(=O)O)C(=O)O)(c1ccc(O)cc1)c1ccc(O)cc1] |
| 101 | 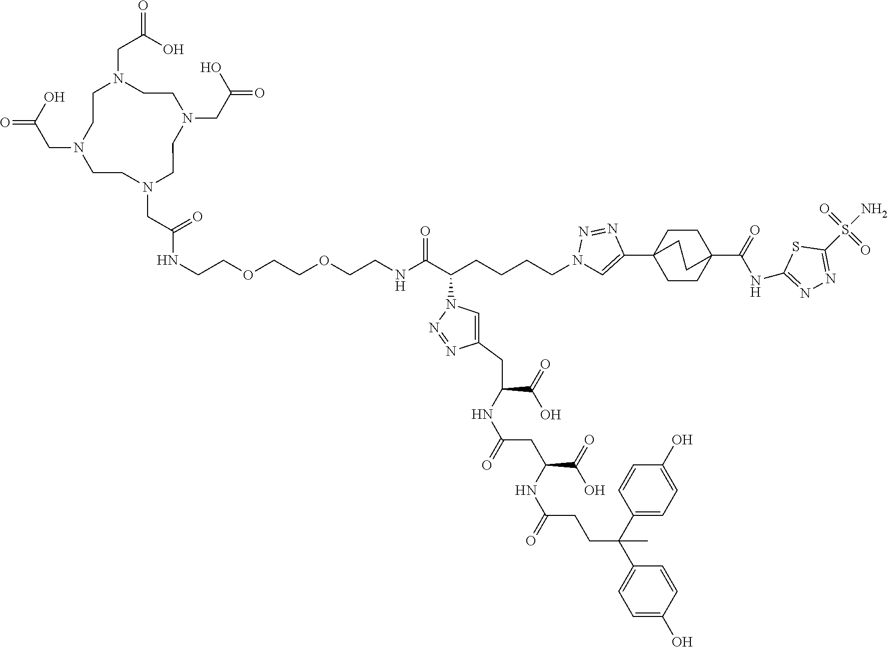 [SMILES: CC(CCC(=O)N[C@@H](CC(=O)N[C@@H](Cc1cn([C@@H](CCCCn2cc(C34CCC(C(=O)Nc5nnc(S(N)(=O)=O)s5)(CC3)CC4)nn2)C(=O)NCCOCCOCCNC(=O)CN2CCN(CC(=O)O)CCN(CC(=O)O)CCN(CC(=O)O)CC2)nn1)C(=O)O)C(=O)O)(c1ccc(O)cc1)c1ccc(O)cc1] |
| 101.1 | 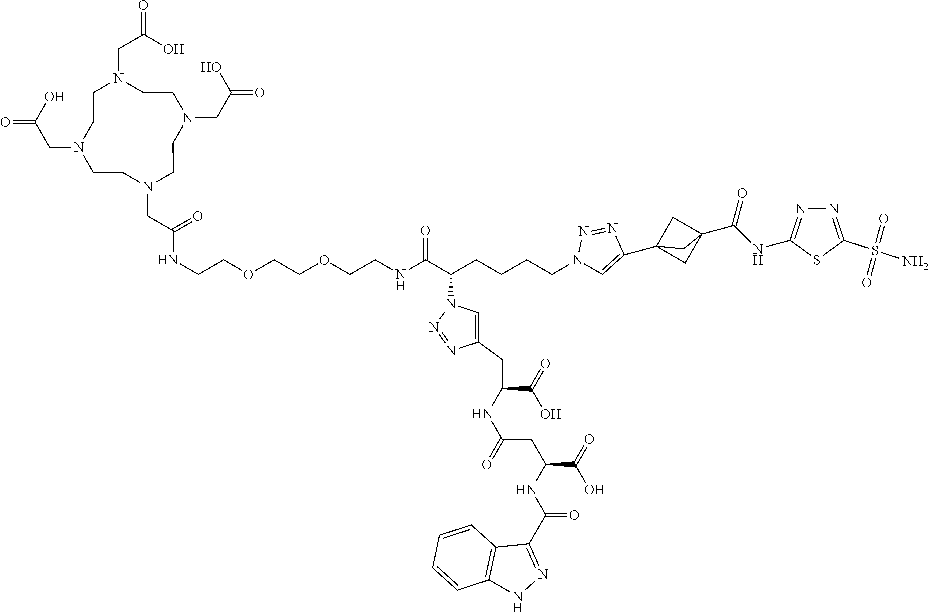 [SMILES: NS(=O)(=O)c1nnc(NC(=O)C23CC(c4cn(CCCC[C@@H](C(=O)NCCOCCOCCNC(=O)CN5CCN(CC(=O)O)CCN(CC(=O)O)CCN(CC(=O)O)CC5)n5cc(C[C@H](NC(=O)C[C@H](NC(=O)c6n[nH]c7ccccc67)C(=O)O)C(=O)O)nn5)nn4)(C2)C3)s1] |
| 101.2 | 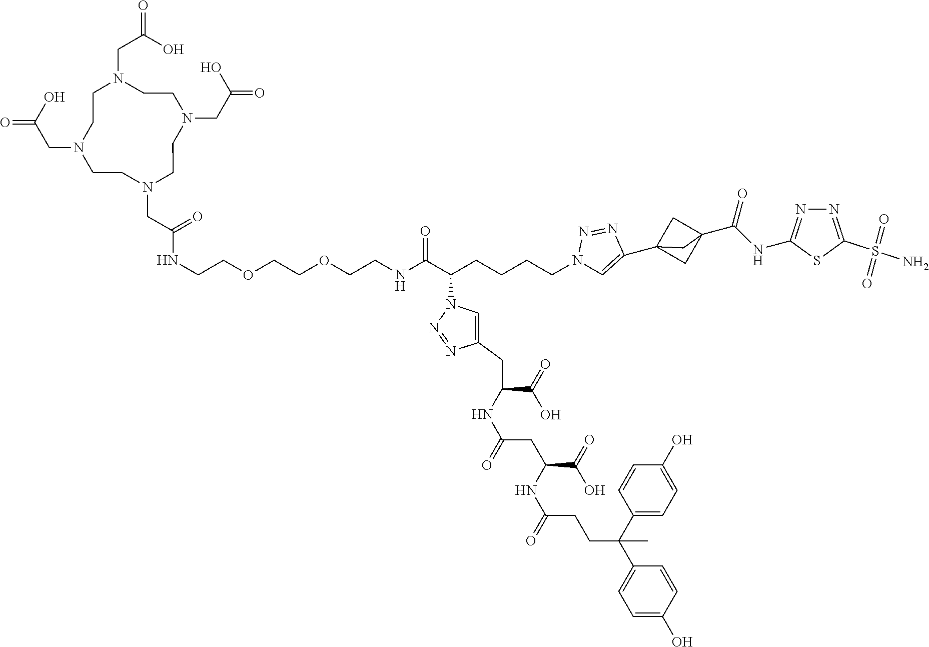 [SMILES: CC(CCC(=O)N[C@@H](CC(=O)N[C@@H](Cc1cn([C@@H](CCCCn2cc(C34CC(C(=O)Nc5nnc(S(N)(=O)=O)s5)(C3)C4)nn2)C(=O)NCCOCCOCCNC(=O)CN2CCN(CC(=O)O)CCN(CC(=O)O)CCN(CC(=O)O)CC2)nn1)C(=O)O)C(=O)O)(c1ccc(O)cc1)c1ccc(O)cc1] |
| 102 | 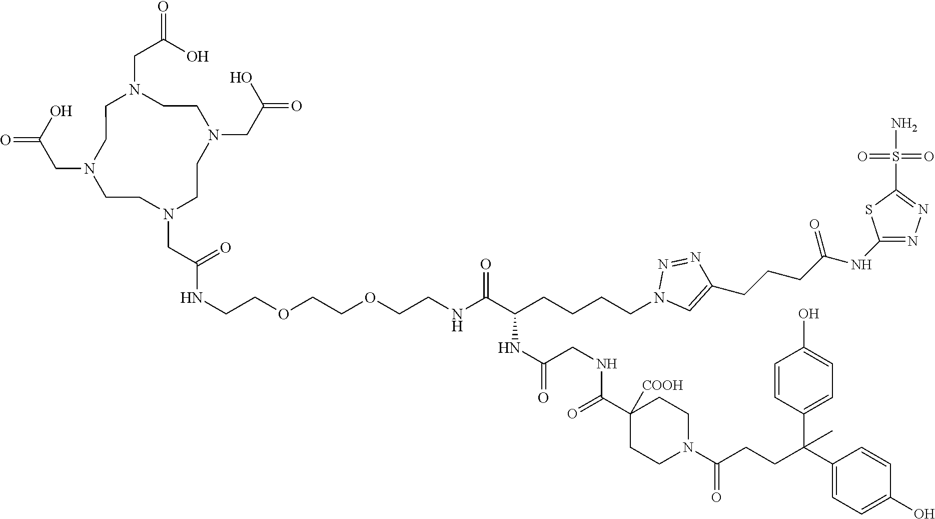 [SMILES: CC(CCC(=O)N1CCC(C(=O)O)(C(=O)NCC(=O)N[C@@H](CCCCn2cc(CCCC(=O)Nc3nnc(S(N)(=O)=O)s3)nn2)C(=O)NCCOCCOCCNC(=O)CN2CCN(CC(=O)O)CCN(CC(=O)O)CCN(CC(=O)O)CC2)CC1)(c1ccc(O)cc1)c1ccc(O)cc1] |
| 102.1 | 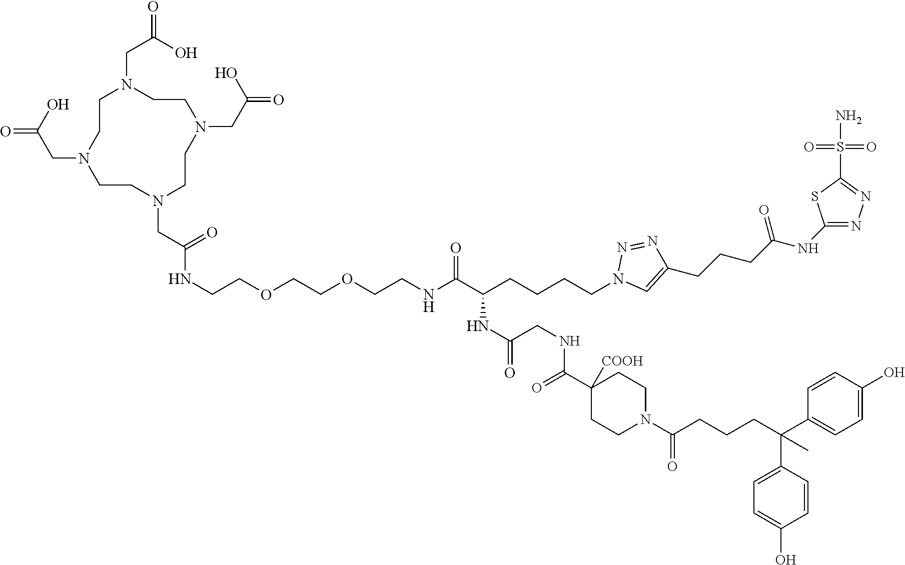 [SMILES: CC(CCCC(=O)N1CCC(C(=O)O)(C(=O)NCC(=O)N[C@@H](CCCCn2cc(CCCC(=O)Nc3nnc(S(N)(=O)=O)s3)nn2)C(=O)NCCOCCOCCNC(=O)CN2CCN(CC(=O)O)CCN(CC(=O)O)CCN(CC(=O)O)CC2)CC1)(c1ccc(O)cc1)c1ccc(O)cc1] |
| 102.2 | 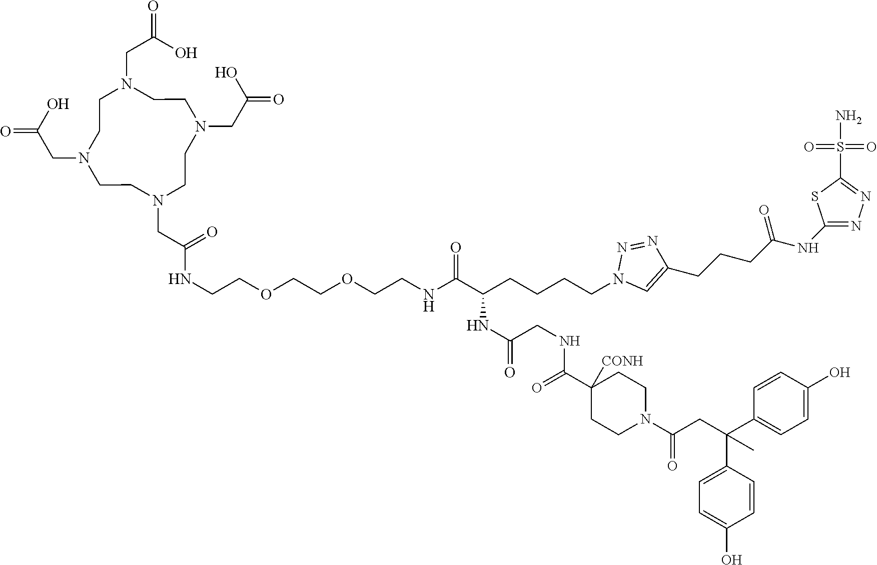 |
| 102.3 | 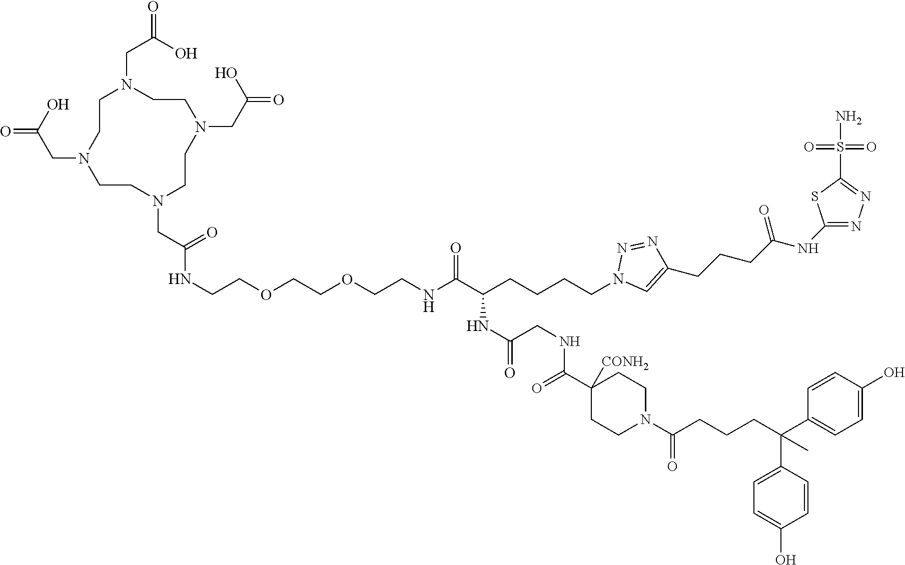 [SMILES: CC(CCCC(=O)N1CCC(C(N)=O)(C(=O)NCC(=O)N[C@@H](CCCCn2cc(CCCC(=O)Nc3nnc(S(N)(=O)=O)s3)nn2)C(=O)NCCOCCOCCNC(=O)CN2CCN(CC(=O)O)CCN(CC(=O)O)CCN(CC(=O)O)CC2)CC1)(c1ccc(O)cc1)c1ccc(O)cc1] |
| 102.4 | 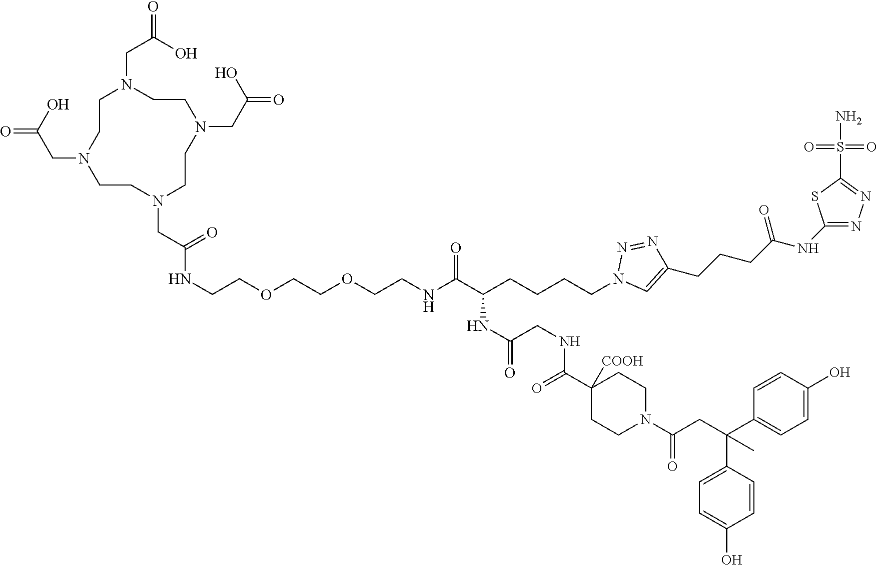 [SMILES: CC(CC(=O)N1CCC(C(=O)O)(C(=O)NCC(=O)N[C@@H](CCCCn2cc(CCCC(=O)Nc3nnc(S(N)(=O)=O)s3)nn2)C(=O)NCCOCCOCCNC(=O)CN2CCN(CC(=O)O)CCN(CC(=O)O)CCN(CC(=O)O)CC2)CC1)(c1ccc(O)cc1)c1ccc(O)cc1] |
| 102.5 | 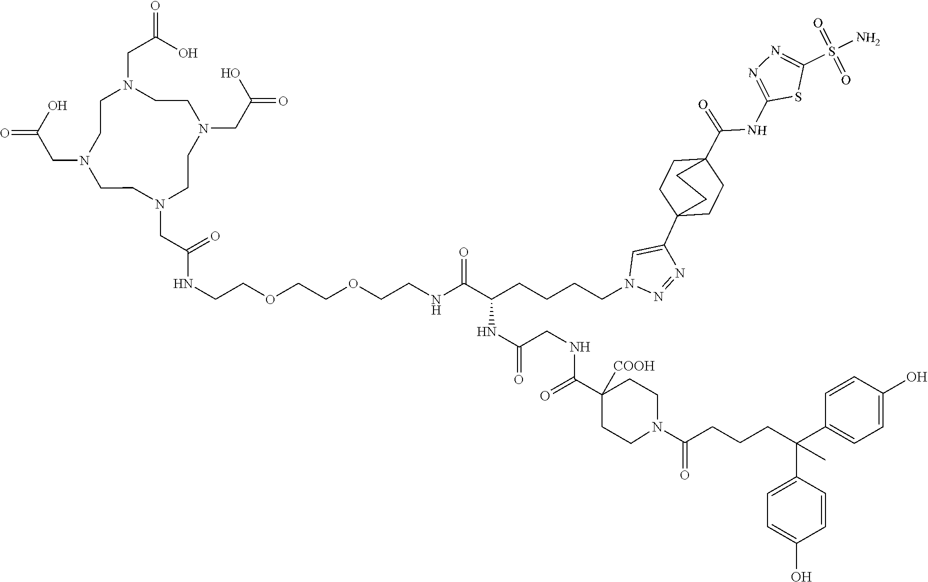 [SMILES: CC(CCCC(=O)N1CCC(C(=O)O)(C(=O)NCC(=O)N[C@@H](CCCCn2cc(C34CCC(C(=O)Nc5nnc(S(N)(=O)=O)s5)(CC3)CC4)nn2)C(=O)NCCOCCOCCNC(=O)CN2CCN(CC(=O)O)CCN(CC(=O)O)CCN(CC(=O)O)CC2)CC1)(c1ccc(O)cc1)c1ccc(O)cc1] |
| 102.6 | 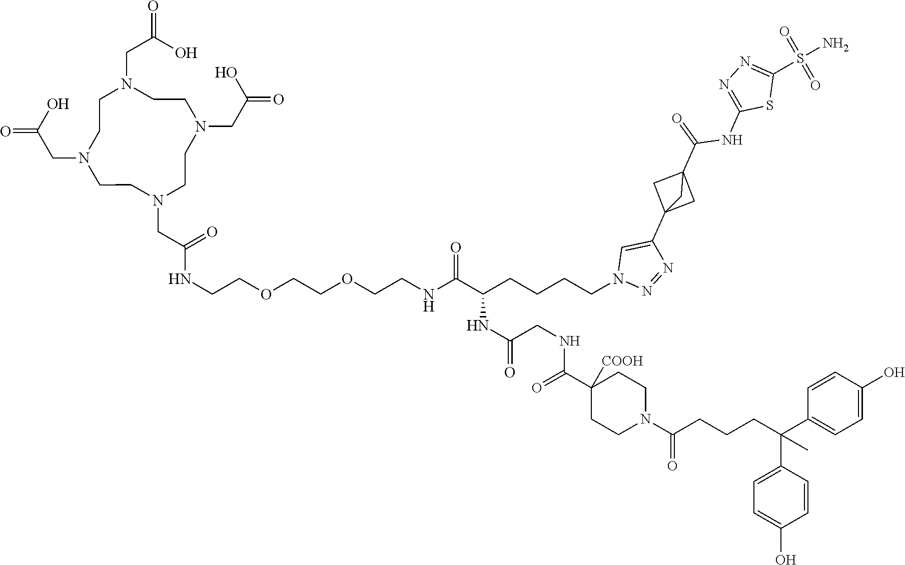 [SMILES: CC(CCCC(=O)N1CCC(C(=O)O)(C(=O)NCC(=O)N[C@@H](CCCCn2cc(C34CC(C(=O)Nc5nnc(S(N)(=O)=O)s5)(C3)C4)nn2)C(=O)NCCOCCOCCNC(=O)CN2CCN(CC(=O)O)CCN(CC(=O)O)CCN(CC(=O)O)CC2)CC1)(c1ccc(O)cc1)c1ccc(O)cc1] |
| 102.7 | 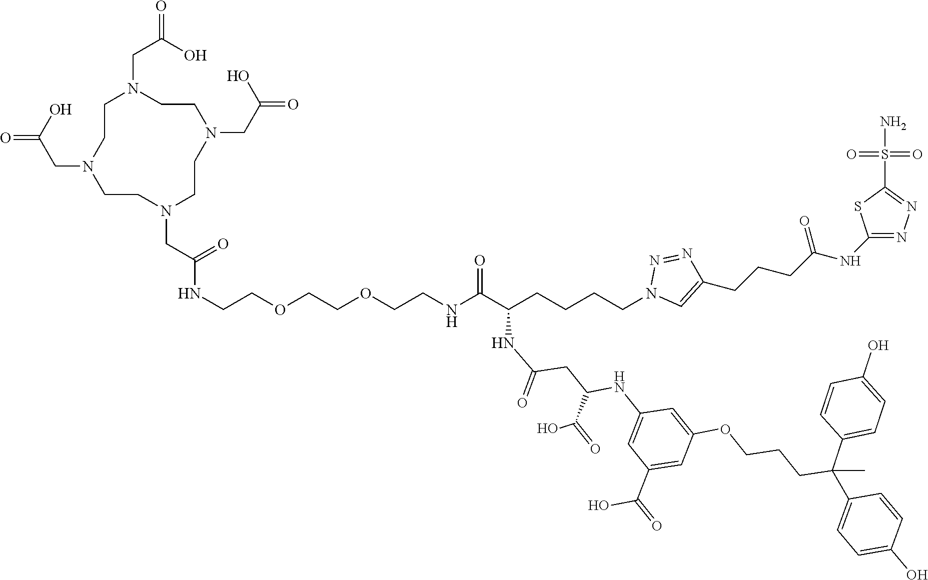 |
| 102.8 | 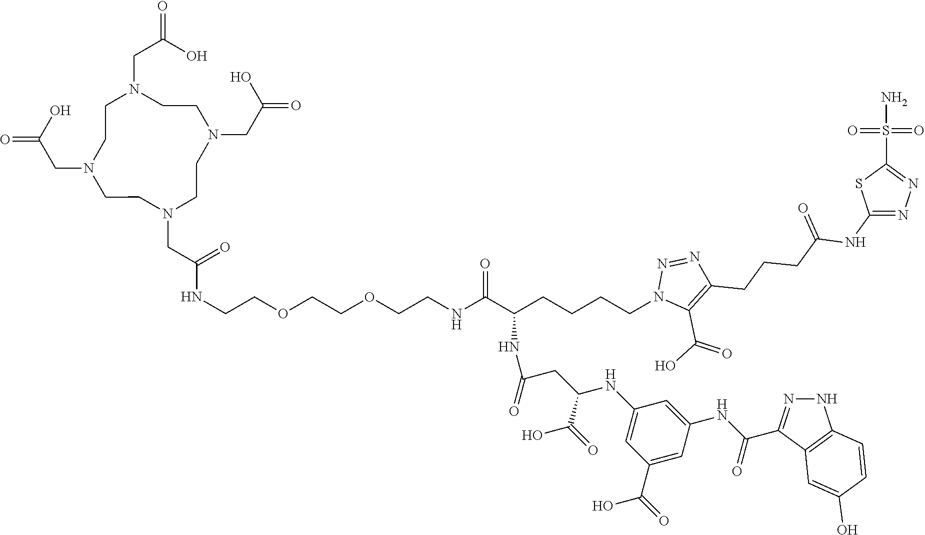 [SMILES: NS(=O)(=O)c1nnc(NC(=O)CCCc2nnn(CCCC[C@H](NC(=O)C[C@H](Nc3cc(NC(=O)c4n[nH]c5ccc(O)cc45)cc(C(=O)O)c3)C(=O)O)C(=O)NCCOCCOCCNC(=O)CN3CCN(CC(=O)O)CCN(CC(=O)O)CCN(CC(=O)O)CC3)c2C(=O)O)s1] |
| 103 | 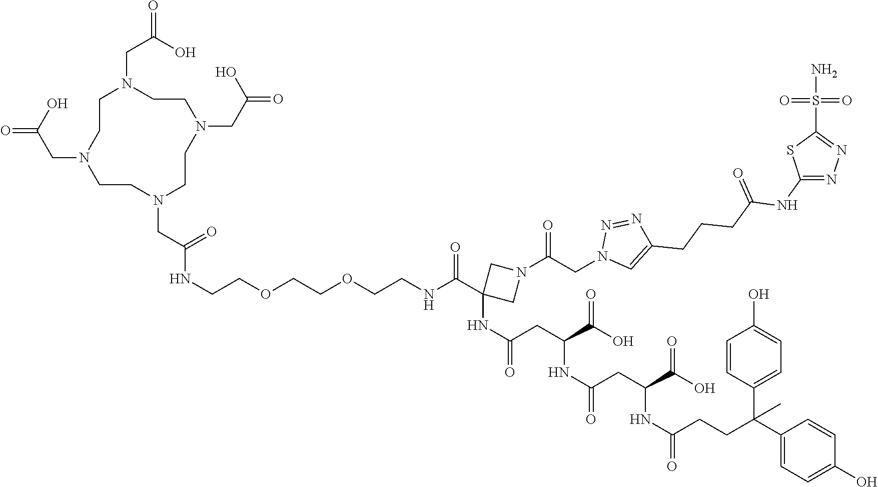 [SMILES: CC(CCC(=O)N[C@@H](CC(=O)N[C@@H](CC(=O)NC1(C(=O)NCCOCCOCCNC(=O)CN2CCN(CC(=O)O)CCN(CC(=O)O)CCN(CC(=O)O)CC2)CN(C(=O)Cn2cc(CCCC(=O)Nc3nnc(S(N)(=O)=O)s3)nn2)C1)C(=O)O)C(=O)O)(c1ccc(O)cc1)c1ccc(O)cc1] |
| 103.1 | 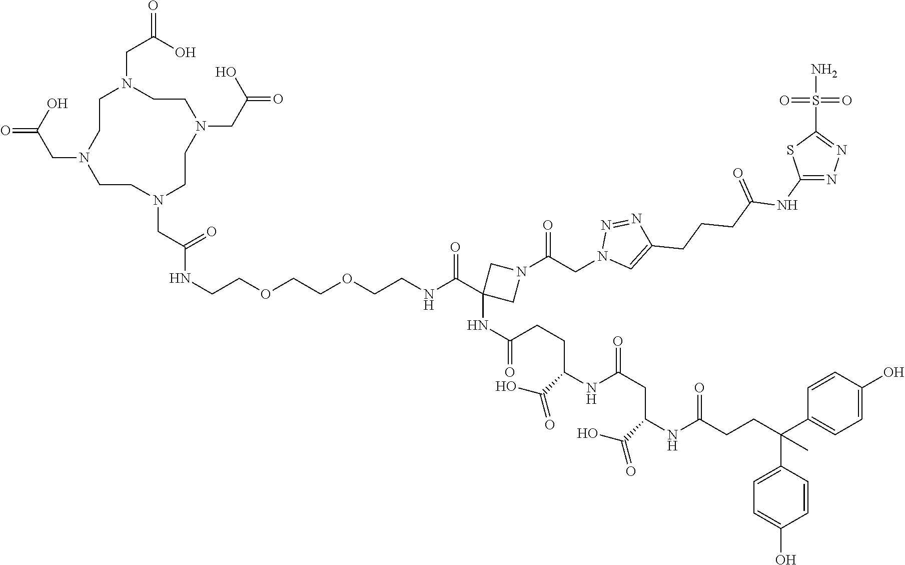 [SMILES: CC(CCC(=O)N[C@@H](CC(=O)N[C@@H](CCC(=O)NC1(C(=O)NCCOCCOCCNC(=O)CN2CCN(CC(=O)O)CCN(CC(=O)O)CCN(CC(=O)O)CC2)CN(C(=O)Cn2cc(CCCC(=O)Nc3nnc(S(N)(=O)=O)s3)nn2)C1)C(=O)O)C(=O)O)(c1ccc(O)cc1)c1ccc(O)cc1] |
| 103.2 | 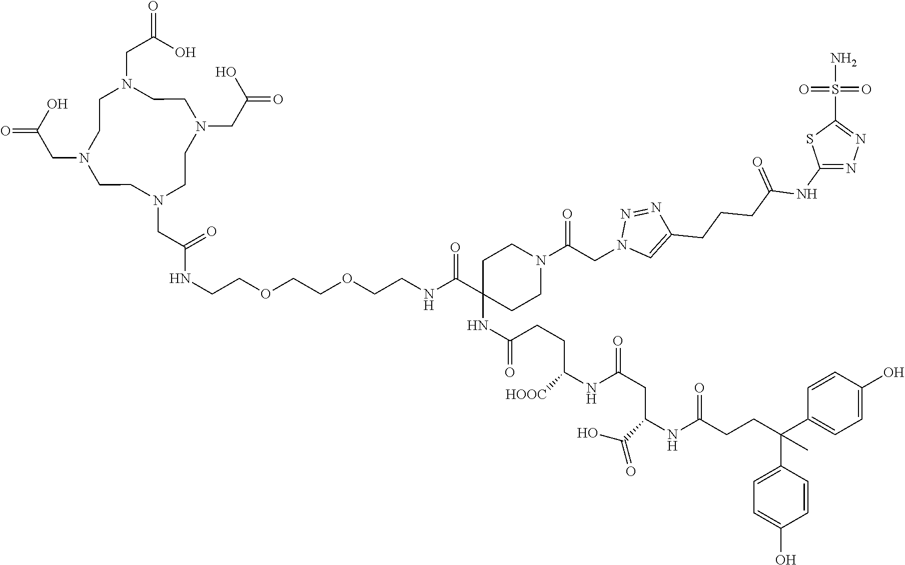 [SMILES: CC(CCC(=O)N[C@@H](CC(=O)N[C@@H](CCC(=O)NC1(C(=O)NCCOCCOCCNC(=O)CN2CCN(CC(=O)O)CCN(CC(=O)O)CCN(CC(=O)O)CC2)CCN(C(=O)Cn2cc(CCCC(=O)Nc3nnc(S(N)(=O)=O)s3)nn2)CC1)C(=O)O)C(=O)O)(c1ccc(O)cc1)c1ccc(O)cc1] |
| 103.3 | 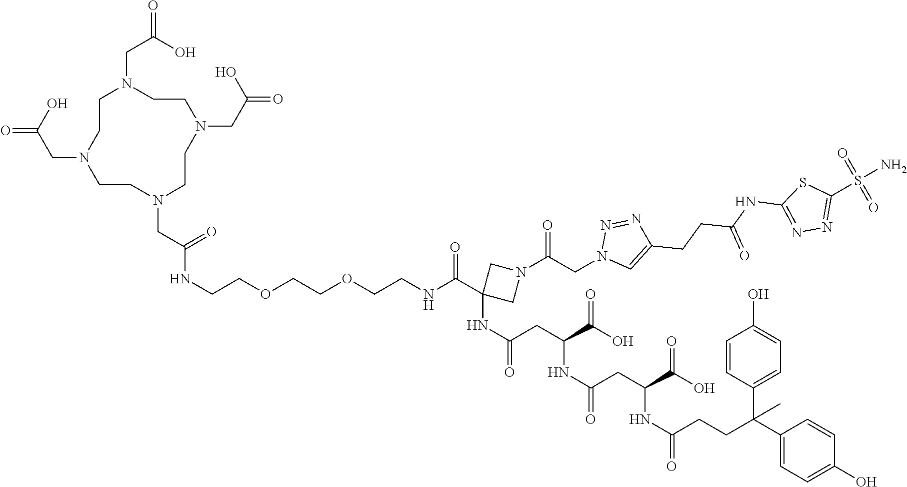 [SMILES: CC(CCC(=O)N[C@@H](CC(=O)N[C@@H](CC(=O)NC1(C(=O)NCCOCCOCCNC(=O)CN2CCN(CC(=O)O)CCN(CC(=O)O)CCN(CC(=O)O)CC2)CN(C(=O)Cn2cc(CCC(=O)Nc3nnc(S(N)(=O)=O)s3)nn2)C1)C(=O)O)C(=O)O)(c1ccc(O)cc1)c1ccc(O)cc1] |
| 103.4 | 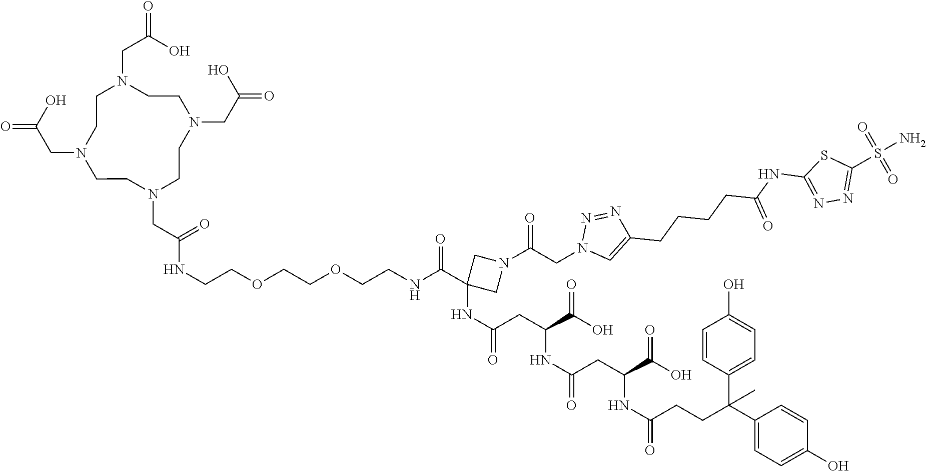 [SMILES: CC(CCC(=O)N[C@@H](CC(=O)N[C@@H](CC(=O)NC1(C(=O)NCCOCCOCCNC(=O)CN2CCN(CC(=O)O)CCN(CC(=O)O)CCN(CC(=O)O)CC2)CN(C(=O)Cn2cc(CCCCC(=O)Nc3nnc(S(N)(=O)=O)s3)nn2)C1)C(=O)O)C(=O)O)(c1ccc(O)cc1)c1ccc(O)cc1] |
| 104 | 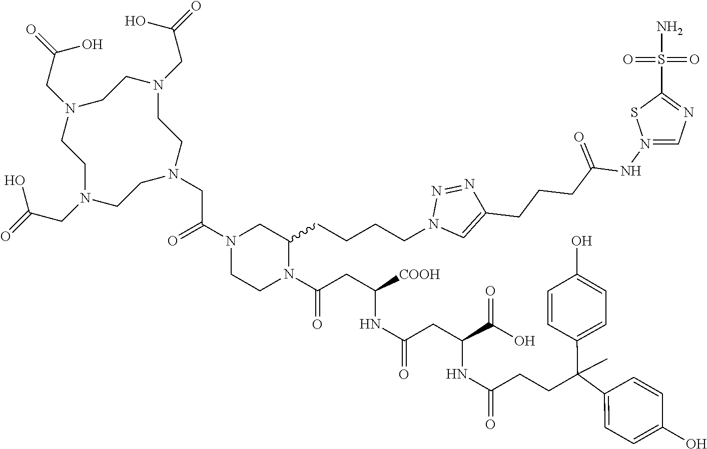 |
| 104.1 | 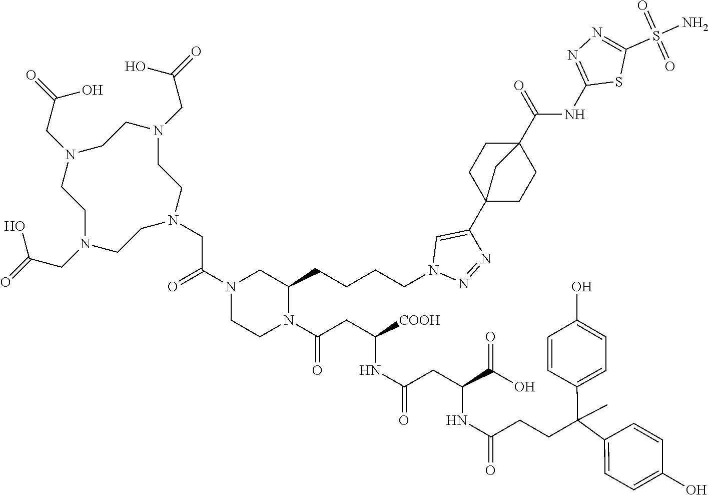 [SMILES: CC(CCC(=O)N[C@@H](CC(=O)N[C@@H](CC(=O)N1CCN(C(=O)CN2CCN(CC(=O)O)CCN(CC(=O)O)CCN(CC(=O)O)CC2)C[C@H]1CCCCn1cc(C23CCC(C(=O)Nc4nnc(S(N)(=O)=O)s4)(CC2)C3)nn1)C(=O)O)C(=O)O)(c1ccc(O)cc1)c1ccc(O)cc1] |
| 104.2 | 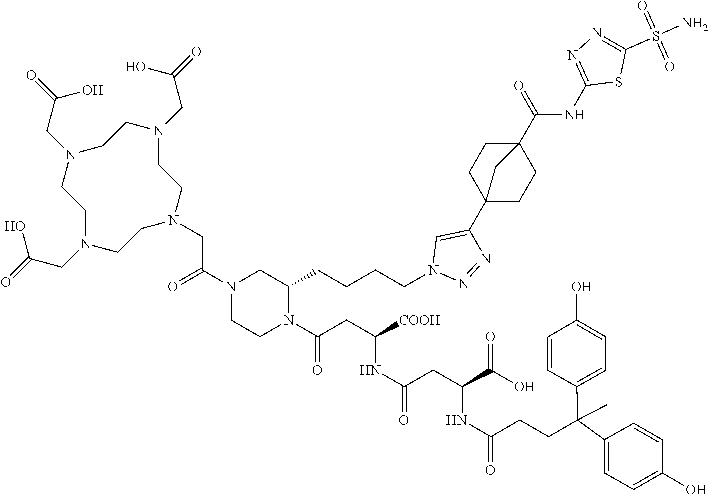 [SMILES: CC(CCC(=O)N[C@@H](CC(=O)N[C@@H](CC(=O)N1CCN(C(=O)CN2CCN(CC(=O)O)CCN(CC(=O)O)CCN(CC(=O)O)CC2)C[C@@H]1CCCCn1cc(C23CCC(C(=O)Nc4nnc(S(N)(=O)=O)s4)(CC2)C3)nn1)C(=O)O)C(=O)O)(c1ccc(O)cc1)c1ccc(O)cc1] |
| 106 | 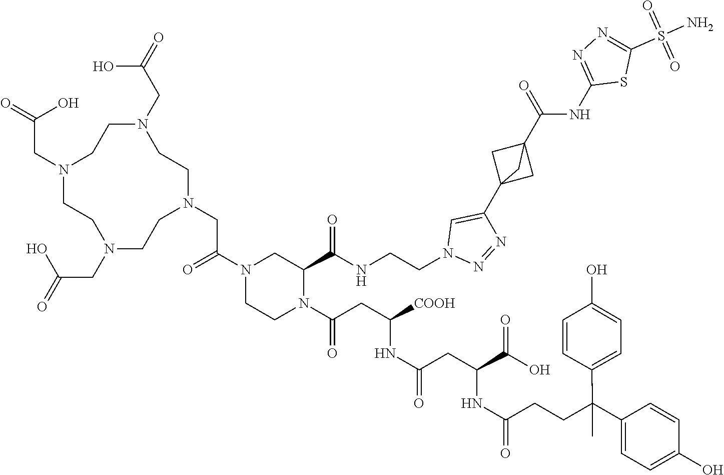 [SMILES: CC(CCC(=O)N[C@@H](CC(=O)N[C@@H](CC(=O)N1CCN(C(=O)CN2CCN(CC(=O)O)CCN(CC(=O)O)CCN(CC(=O)O)CC2)C[C@H]1C(=O)NCCn1cc(C23CC(C(=O)Nc4nnc(S(N)(=O)=O)s4)(C2)C3)nn1)C(=O)O)C(=O)O)(c1ccc(O)cc1)c1ccc(O)cc1] |
| 107 | 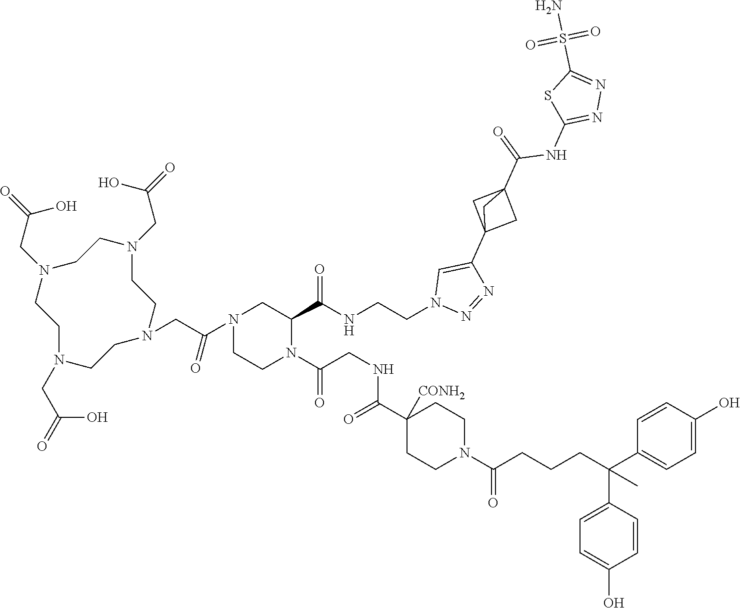 [SMILES: CC(CCCC(=O)N1CCC(C(N)=O)(C(=O)NCC(=O)N2CCN(C(=O)CN3CCN(CC(=O)O)CCN(CC(=O)O)CCN(CC(=O)O)CC3)C[C@H]2C(=O)NCCn2cc(C34CC(C(=O)Nc5nnc(S(N)(=O)=O)s5)(C3)C4)nn2)CC1)(c1ccc(O)cc1)c1ccc(O)cc1] |
| 107.1 | 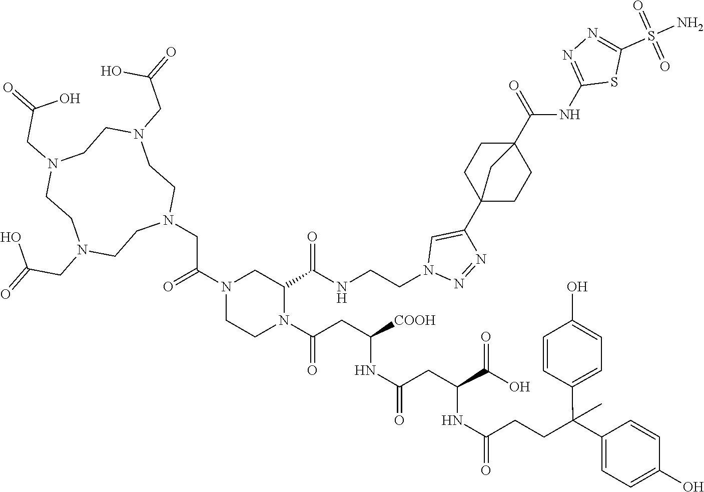 |
| 107.2 | 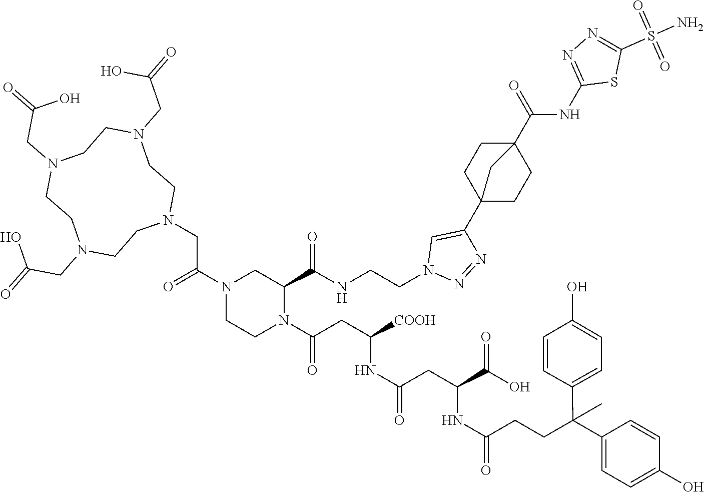 [SMILES: CC(CCC(=O)N[C@@H](CC(=O)N[C@@H](CC(=O)N1CCN(C(=O)CN2CCN(CC(=O)O)CCN(CC(=O)O)CCN(CC(=O)O)CC2)C[C@H]1C(=O)NCCn1cc(C23CCC(C(=O)Nc4nnc(S(N)(=O)=O)s4)(CC2)C3)nn1)C(=O)O)C(=O)O)(c1ccc(O)cc1)c1ccc(O)cc1] |
| 107.3 | 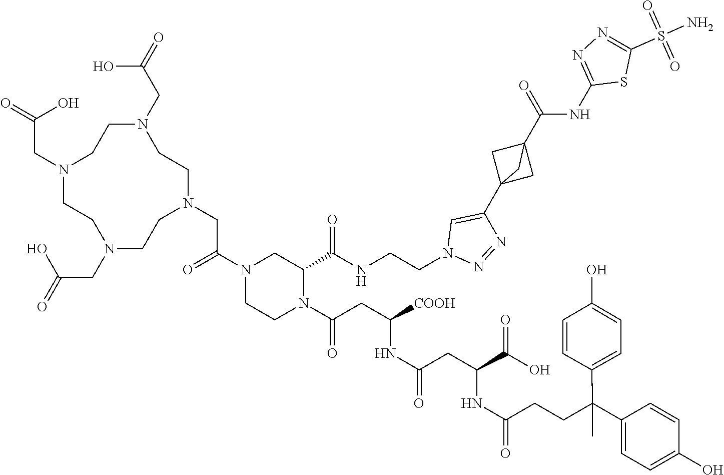 [SMILES: CC(CCC(=O)N[C@@H](CC(=O)N[C@@H](CC(=O)N1CCN(C(=O)CN2CCN(CC(=O)O)CCN(CC(=O)O)CCN(CC(=O)O)CC2)C[C@@H]1C(=O)NCCn1cc(C23CC(C(=O)Nc4nnc(S(N)(=O)=O)s4)(C2)C3)nn1)C(=O)O)C(=O)O)(c1ccc(O)cc1)c1ccc(O)cc1] |
| 107.4 | 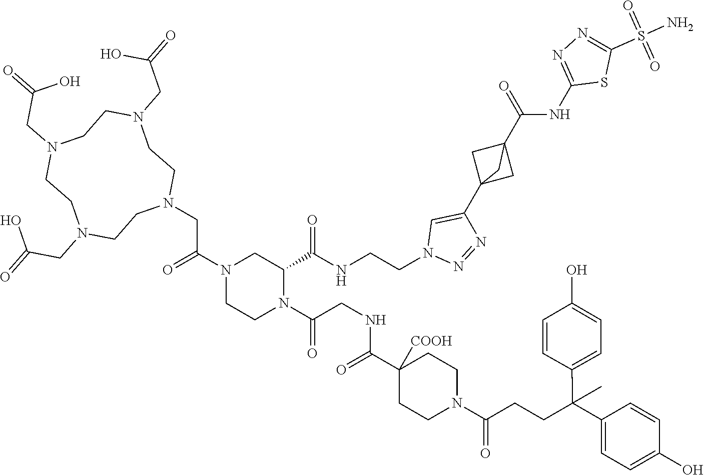 [SMILES: CC(CCC(=O)N1CCC(C(=O)O)(C(=O)NCC(=O)N2CCN(C(=O)CN3CCN(CC(=O)O)CCN(CC(=O)O)CCN(CC(=O)O)CC3)C[C@@H]2C(=O)NCCn2cc(C34CC(C(=O)Nc5nnc(S(N)(=O)=O)s5)(C3)C4)nn2)CC1)(c1ccc(O)cc1)c1ccc(O)cc1] |
| 107.5 | 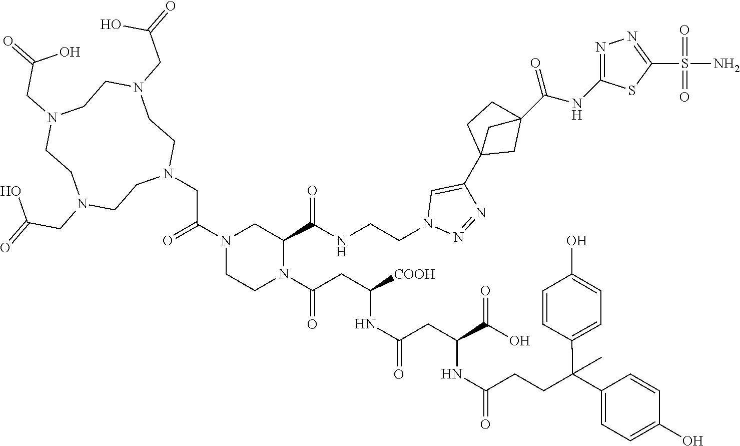 [SMILES: CC(CCC(=O)N[C@@H](CC(=O)N[C@@H](CC(=O)N1CCN(C(=O)CN2CCN(CC(=O)O)CCN(CC(=O)O)CCN(CC(=O)O)CC2)C[C@H]1C(=O)NCCn1cc(C23CCC(C(=O)Nc4nnc(S(N)(=O)=O)s4)(C2)C3)nn1)C(=O)O)C(=O)O)(c1ccc(O)cc1)c1ccc(O)cc1] |
| 107.6 | 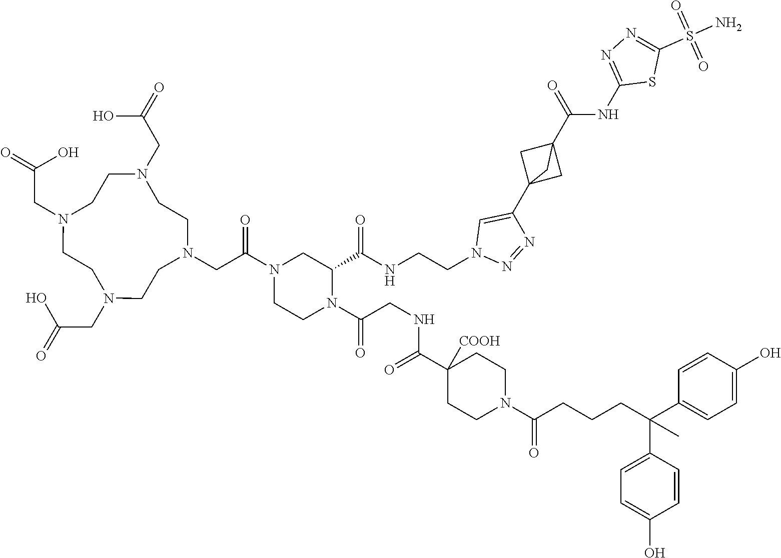 [SMILES: CC(CCCC(=O)N1CCC(C(=O)O)(C(=O)NCC(=O)N2CCN(C(=O)CN3CCN(CC(=O)O)CCN(CC(=O)O)CCN(CC(=O)O)CC3)C[C@@H]2C(=O)NCCn2cc(C34CC(C(=O)Nc5nnc(S(N)(=O)=O)s5)(C3)C4)nn2)CC1)(c1ccc(O)cc1)c1ccc(O)cc1] |
| 107.7 | 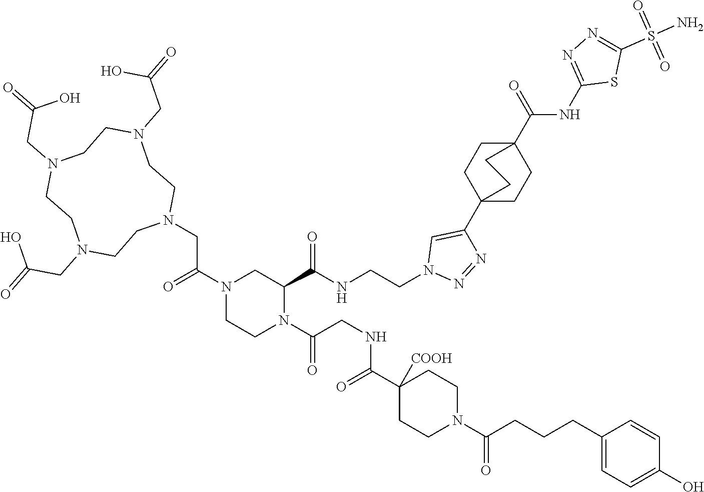 [SMILES: NS(=O)(=O)c1nnc(NC(=O)C23CCC(c4cn(CCNC(=O)[C@@H]5CN(C(=O)CN6CCN(CC(=O)O)CCN(CC(=O)O)CCN(CC(=O)O)CC6)CCN5C(=O)CNC(=O)C5(C(=O)O)CCN(C(=O)CCCc6ccc(O)cc6)CC5)nn4)(CC2)CC3)s1] |
| 107.8 | 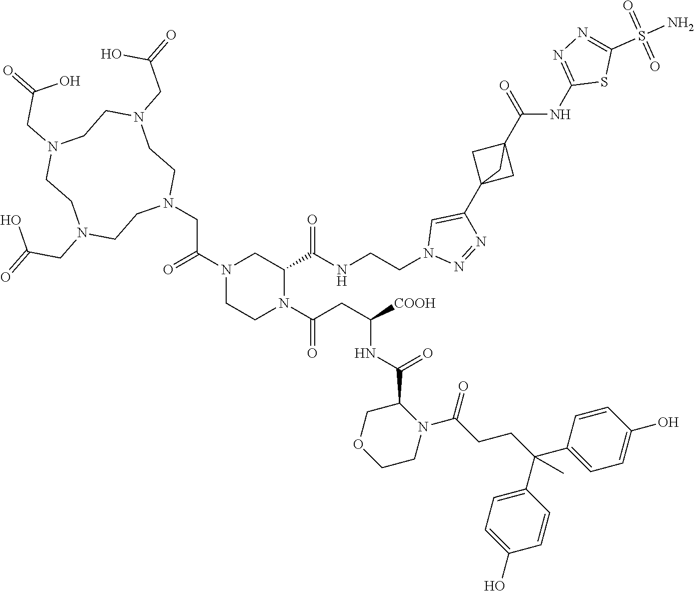 [SMILES: CC(CCC(=O)N1CCOC[C@H]1C(=O)N[C@@H](CC(=O)N1CCN(C(=O)CN2CCN(CC(=O)O)CCN(CC(=O)O)CCN(CC(=O)O)CC2)C[C@@H]1C(=O)NCCn1cc(C23CC(C(=O)Nc4nnc(S(N)(=O)=O)s4)(C2)C3)nn1)C(=O)O)(c1ccc(O)cc1)c1ccc(O)cc1] |
| 107.9 | 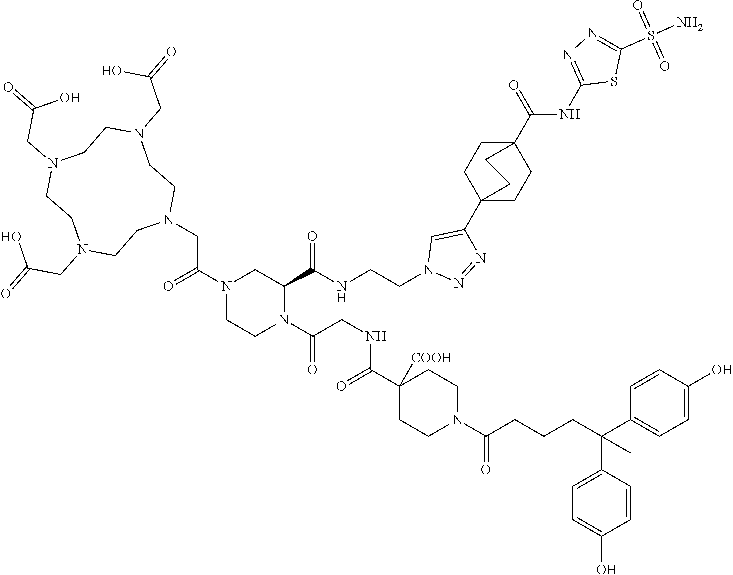 [SMILES: CC(CCCC(=O)N1CCC(C(=O)O)(C(=O)NCC(=O)N2CCN(C(=O)CN3CCN(CC(=O)O)CCN(CC(=O)O)CCN(CC(=O)O)CC3)C[C@H]2C(=O)NCCn2cc(C34CCC(C(=O)Nc5nnc(S(N)(=O)=O)s5)(CC3)CC4)nn2)CC1)(c1ccc(O)cc1)c1ccc(O)cc1] |
| 107.10 | 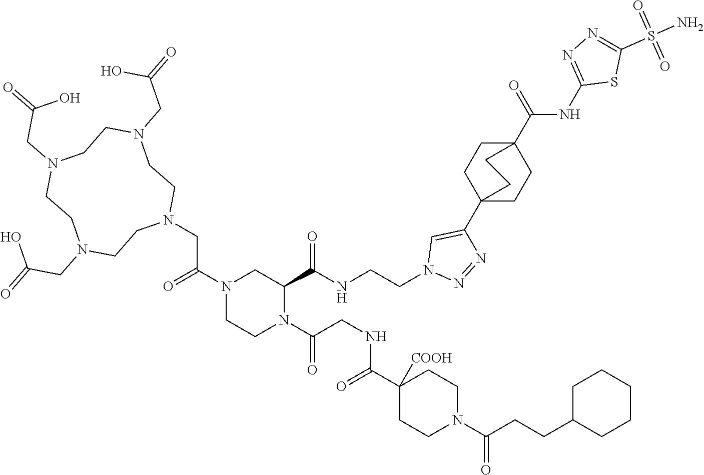 [SMILES: NS(=O)(=O)c1nnc(NC(=O)C23CCC(c4cn(CCNC(=O)[C@@H]5CN(C(=O)CN6CCN(CC(=O)O)CCN(CC(=O)O)CCN(CC(=O)O)CC6)CCN5C(=O)CNC(=O)C5(C(=O)O)CCN(C(=O)CCC6CCCCC6)CC5)nn4)(CC2)CC3)s1] |
| 107.11 | 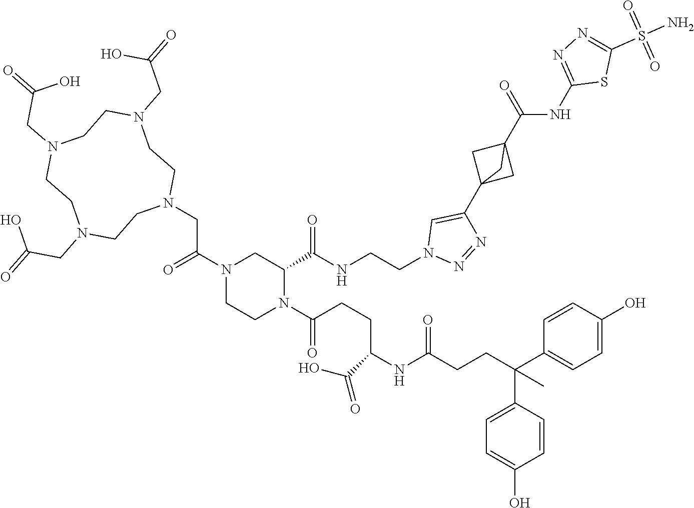 [SMILES: CC(CCC(=O)N[C@@H](CCC(=O)N1CCN(C(=O)CN2CCN(CC(=O)O)CCN(CC(=O)O)CCN(CC(=O)O)CC2)C[C@@H]1C(=O)NCCn1cc(C23CC(C(=O)Nc4nnc(S(N)(=O)=O)s4)(C2)C3)nn1)C(=O)O)(c1ccc(O)cc1)c1ccc(O)cc1] |
| 107.12 | 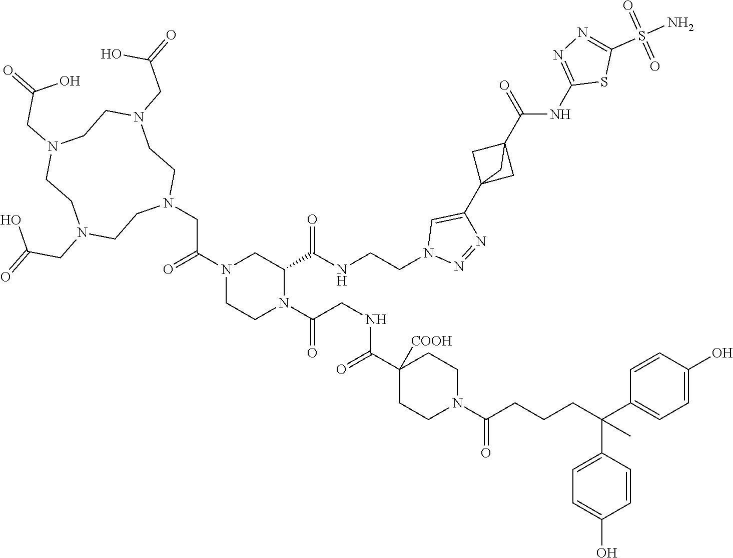 [SMILES: CC(CCCC(=O)N1CCC(C(=O)O)(C(=O)NCC(=O)N2CCN(C(=O)CN3CCN(CC(=O)O)CCN(CC(=O)O)CCN(CC(=O)O)CC3)C[C@@H]2C(=O)NCCn2cc(C34CC(C(=O)Nc5nnc(S(N)(=O)=O)s5)(C3)C4)nn2)CC1)(c1ccc(O)cc1)c1ccc(O)cc1] |
| 107.13 | 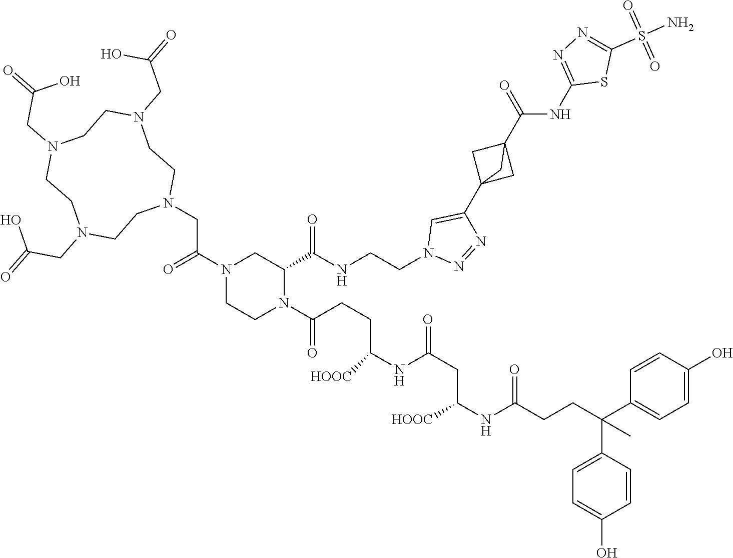 [SMILES: CC(CCC(=O)N[C@@H](CC(=O)N[C@@H](CCC(=O)N1CCN(C(=O)CN2CCN(CC(=O)O)CCN(CC(=O)O)CCN(CC(=O)O)CC2)C[C@@H]1C(=O)NCCn1cc(C23CC(C(=O)Nc4nnc(S(N)(=O)=O)s4)(C2)C3)nn1)C(=O)O)C(=O)O)(c1ccc(O)cc1)c1ccc(O)cc1] |
| 107.14 | 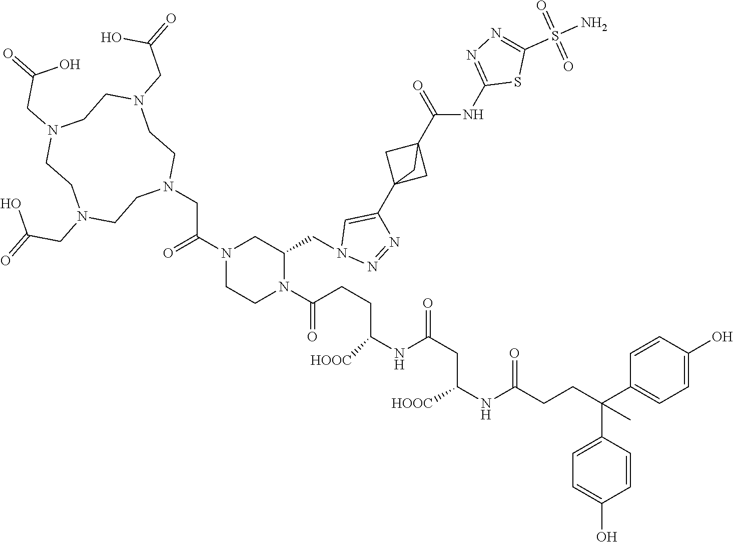 [SMILES: CC(CCC(=O)N[C@@H](CC(=O)N[C@@H](CCC(=O)N1CCN(C(=O)CN2CCN(CC(=O)O)CCN(CC(=O)O)CCN(CC(=O)O)CC2)C[C@@H]1Cn1cc(C23CC(C(=O)Nc4nnc(S(N)(=O)=O)s4)(C2)C3)nn1)C(=O)O)C(=O)O)(c1ccc(O)cc1)c1ccc(O)cc1] |
| 107.15 | 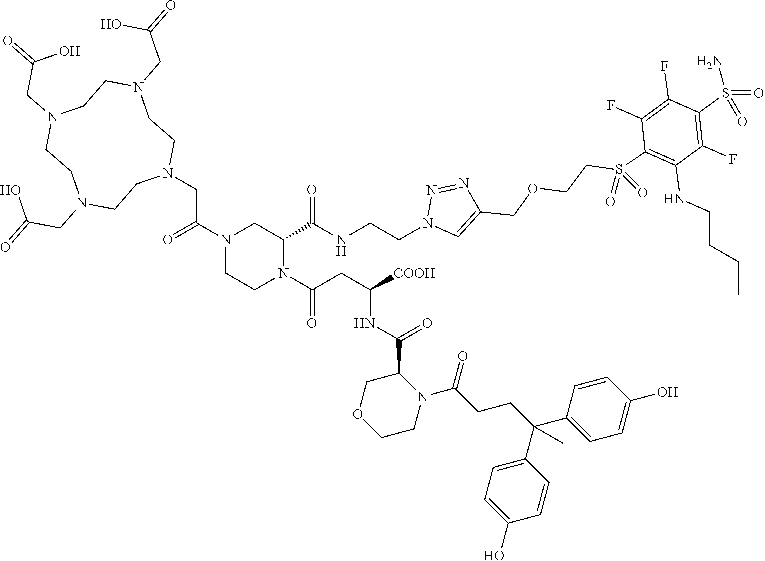 [SMILES: CCCCNc1c(F)c(S(N)(=O)=O)c(F)c(F)c1S(=O)(=O)CCOCc1cn(CCNC(=O)[C@H]2CN(C(=O)CN3CCN(CC(=O)O)CCN(CC(=O)O)CCN(CC(=O)O)CC3)CCN2C(=O)C[C@H](NC(=O)[C@@H]2COCCN2C(=O)CCC(C)(c2ccc(O)cc2)c2ccc(O)cc2)C(=O)O)nn1] |
| 107.16 | 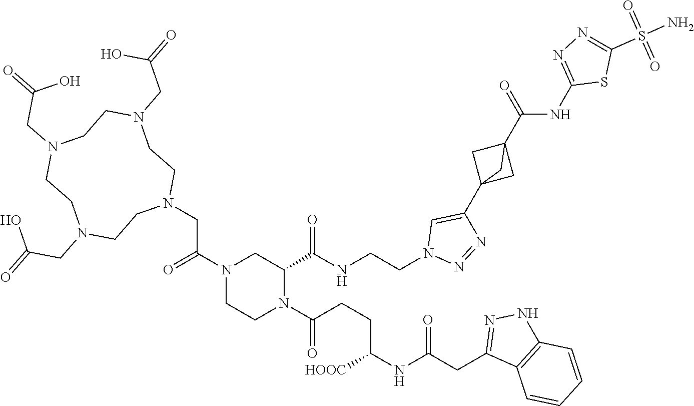 [SMILES: NS(=O)(=O)c1nnc(NC(=O)C23CC(c4cn(CCNC(=O)[C@H]5CN(C(=O)CN6CCN(CC(=O)O)CCN(CC(=O)O)CCN(CC(=O)O)CC6)CCN5C(=O)CC[C@H](NC(=O)Cc5n[nH]c6ccccc56)C(=O)O)nn4)(C2)C3)s1] |
| 107.17 | 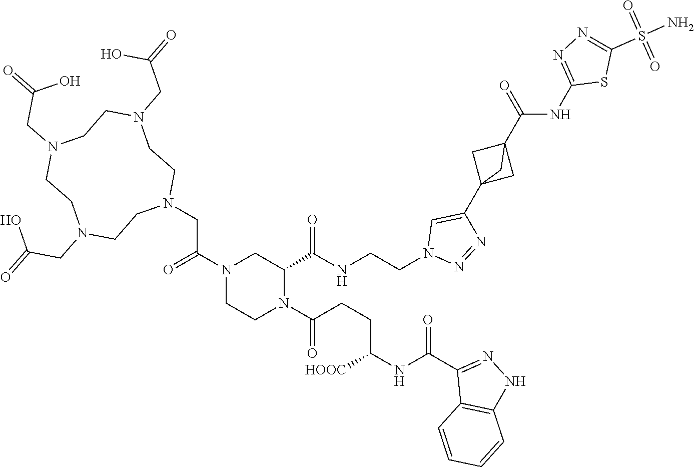 [SMILES: NS(=O)(=O)c1nnc(NC(=O)C23CC(c4cn(CCNC(=O)[C@H]5CN(C(=O)CN6CCN(CC(=O)O)CCN(CC(=O)O)CCN(CC(=O)O)CC6)CCN5C(=O)CC[C@H](NC(=O)c5n[nH]c6ccccc56)C(=O)O)nn4)(C2)C3)s1] |
| 107.18 | 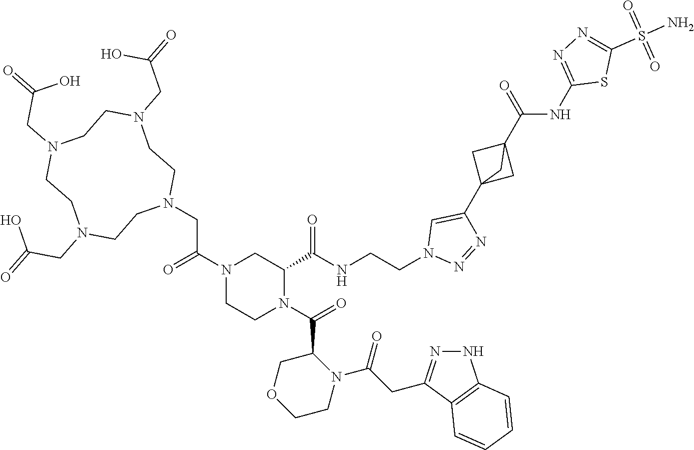 [SMILES: NS(=O)(=O)c1nnc(NC(=O)C23CC(c4cn(CCNC(=O)[C@H]5CN(C(=O)CN6CCN(CC(=O)O)CCN(CC(=O)O)CCN(CC(=O)O)CC6)CCN5C(=O)[C@@H]5COCCN5C(=O)Cc5n[nH]c6ccccc56)nn4)(C2)C3)s1] |
| 107.19 | 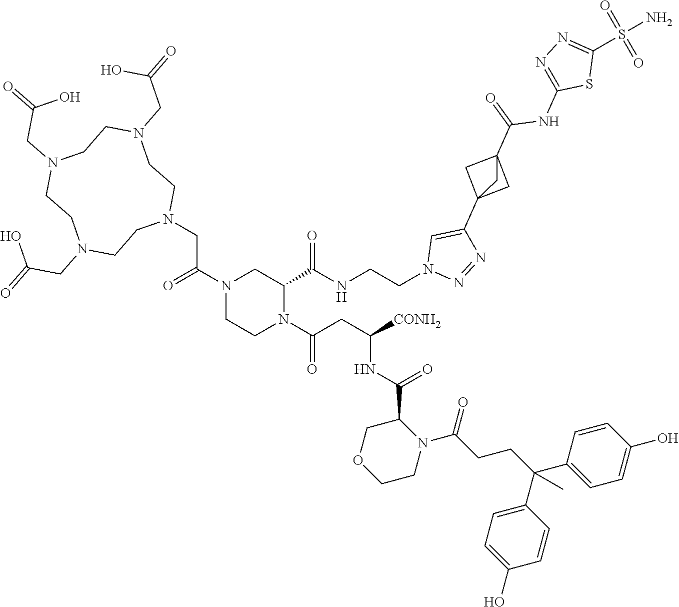 [SMILES: CC(CCC(=O)N1CCOC[C@H]1C(=O)N[C@@H](CC(=O)N1CCN(C(=O)CN2CCN(CC(=O)O)CCN(CC(=O)O)CCN(CC(=O)O)CC2)C[C@@H]1C(=O)NCCn1cc(C23CC(C(=O)Nc4nnc(S(N)(=O)=O)s4)(C2)C3)nn1)C(N)=O)(c1ccc(O)cc1)c1ccc(O)cc1] |
| 107.20 | 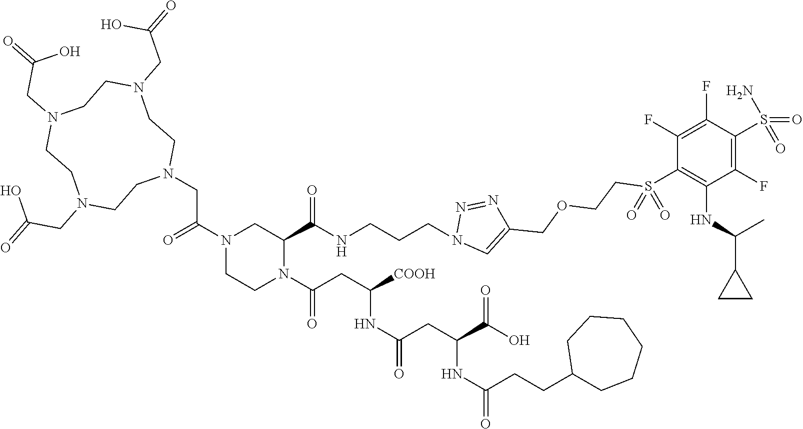 [SMILES: C[C@H](Nc1c(F)c(S(N)(=O)=O)c(F)c(F)c1S(=O)(=O)CCOCc1cn(CCCNC(=O)[C@@H]2CN(C(=O)CN3CCN(CC(=O)O)CCN(CC(=O)O)CCN(CC(=O)O)CC3)CCN2C(=O)C[C@H](NC(=O)C[C@H](NC(=O)CCC2CCCCCC2)C(=O)O)C(=O)O)nn1)C1CC1] |
| 107.21 | 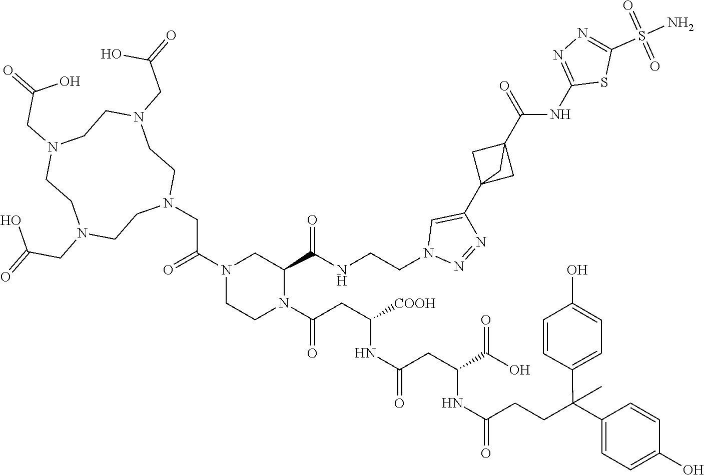 [SMILES: CC(CCC(=O)N[C@H](CC(=O)N[C@H](CC(=O)N1CCN(C(=O)CN2CCN(CC(=O)O)CCN(CC(=O)O)CCN(CC(=O)O)CC2)C[C@H]1C(=O)NCCn1cc(C23CC(C(=O)Nc4nnc(S(N)(=O)=O)s4)(C2)C3)nn1)C(=O)O)C(=O)O)(c1ccc(O)cc1)c1ccc(O)cc1] |
| 107.22 | 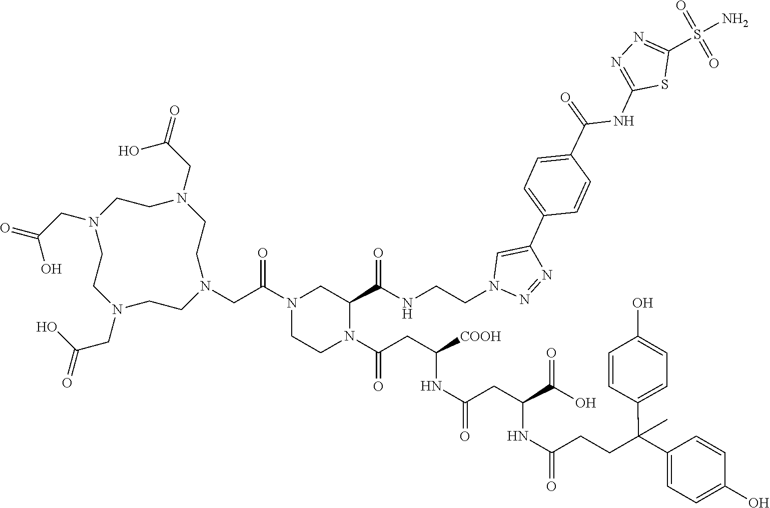 [SMILES: CC(CCC(=O)N[C@@H](CC(=O)N[C@@H](CC(=O)N1CCN(C(=O)CN2CCN(CC(=O)O)CCN(CC(=O)O)CCN(CC(=O)O)CC2)C[C@H]1C(=O)NCCn1cc(-c2ccc(C(=O)Nc3nnc(S(N)(=O)=O)s3)cc2)nn1)C(=O)O)C(=O)O)(c1ccc(O)cc1)c1ccc(O)cc1] |
| 107.23 | 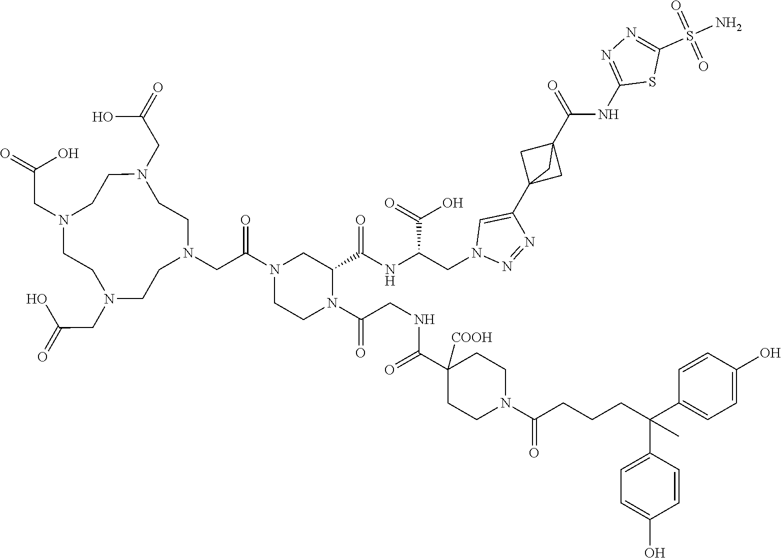 [SMILES: CC(CCCC(=O)N1CCC(C(=O)O)(C(=O)NCC(=O)N2CCN(C(=O)CN3CCN(CC(=O)O)CCN(CC(=O)O)CCN(CC(=O)O)CC3)C[C@@H]2C(=O)N[C@@H](Cn2cc(C34CC(C(=O)Nc5nnc(S(N)(=O)=O)s5)(C3)C4)nn2)C(=O)O)CC1)(c1ccc(O)cc1)c1ccc(O)cc1] |
| 107.24 | 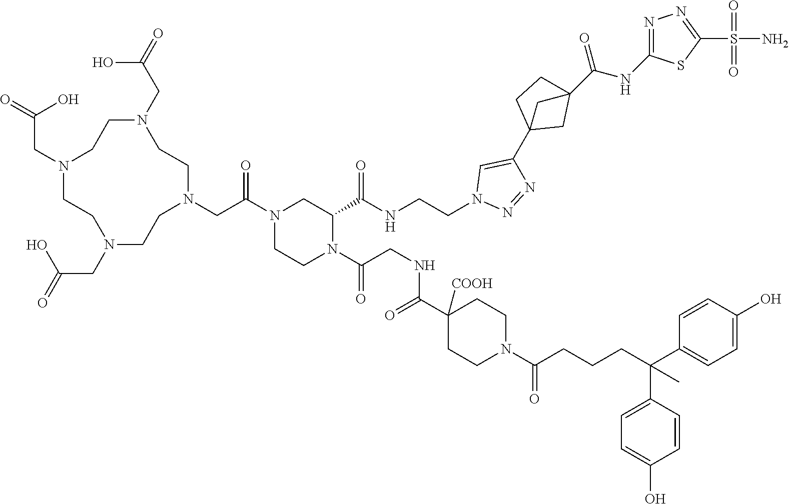 [SMILES: CC(CCCC(=O)N1CCC(C(=O)O)(C(=O)NCC(=O)N2CCN(C(=O)CN3CCN(CC(=O)O)CCN(CC(=O)O)CCN(CC(=O)O)CC3)C[C@@H]2C(=O)NCCn2cc(C34CCC(C(=O)Nc5nnc(S(N)(=O)=O)s5)(C3)C4)nn2)CC1)(c1ccc(O)cc1)c1ccc(O)cc1] |
| 107.25 | 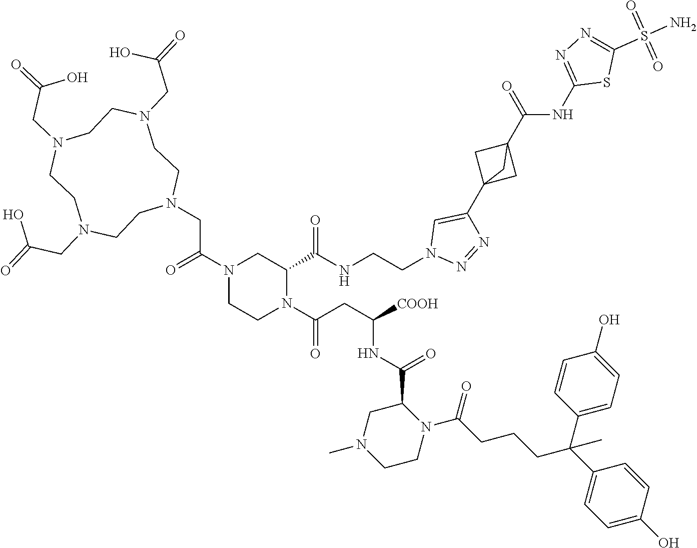 [SMILES: CN1CCN(C(=O)CCCC(C)(c2ccc(O)cc2)c2ccc(O)cc2)[C@H](C(=O)N[C@@H](CC(=O)N2CCN(C(=O)CN3CCN(CC(=O)O)CCN(CC(=O)O)CCN(CC(=O)O)CC3)C[C@@H]2C(=O)NCCn2cc(C34CC(C(=O)Nc5nnc(S(N)(=O)=O)s5)(C3)C4)nn2)C(=O)O)C1] |
| 107.26 | 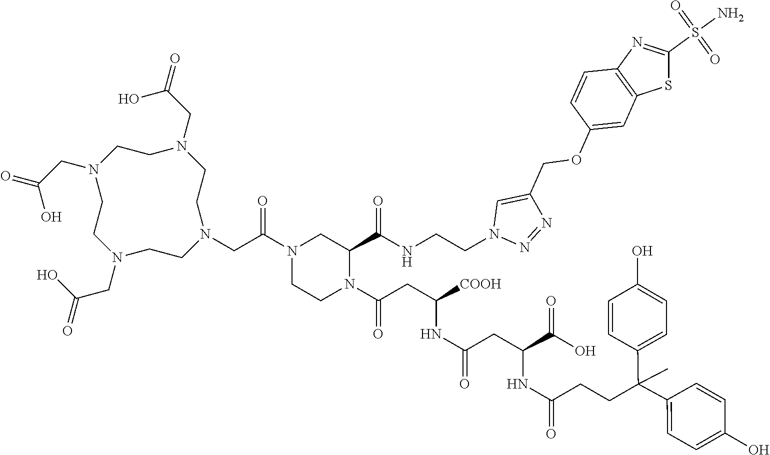 [SMILES: CC(CCC(=O)N[C@@H](CC(=O)N[C@@H](CC(=O)N1CCN(C(=O)CN2CCN(CC(=O)O)CCN(CC(=O)O)CCN(CC(=O)O)CC2)C[C@H]1C(=O)NCCn1cc(COc2ccc3nc(S(N)(=O)=O)sc3c2)nn1)C(=O)O)C(=O)O)(c1ccc(O)cc1)c1ccc(O)cc1] |
| 107.27 | 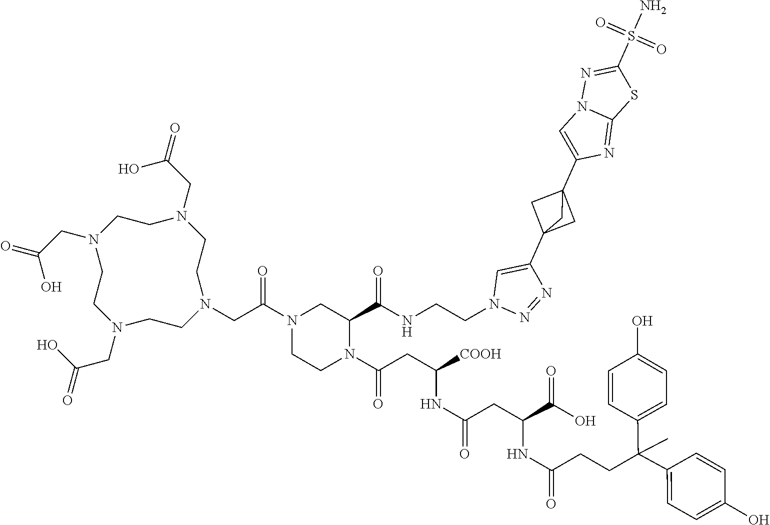 [SMILES: CC(CCC(=O)N[C@@H](CC(=O)N[C@@H](CC(=O)N1CCN(C(=O)CN2CCN(CC(=O)O)CCN(CC(=O)O)CCN(CC(=O)O)CC2)C[C@H]1C(=O)NCCn1cc(C23CC(c4cn5nc(S(N)(=O)=O)sc5n4)(C2)C3)nn1)C(=O)O)C(=O)O)(c1ccc(O)cc1)c1ccc(O)cc1] |
| 107.28 | 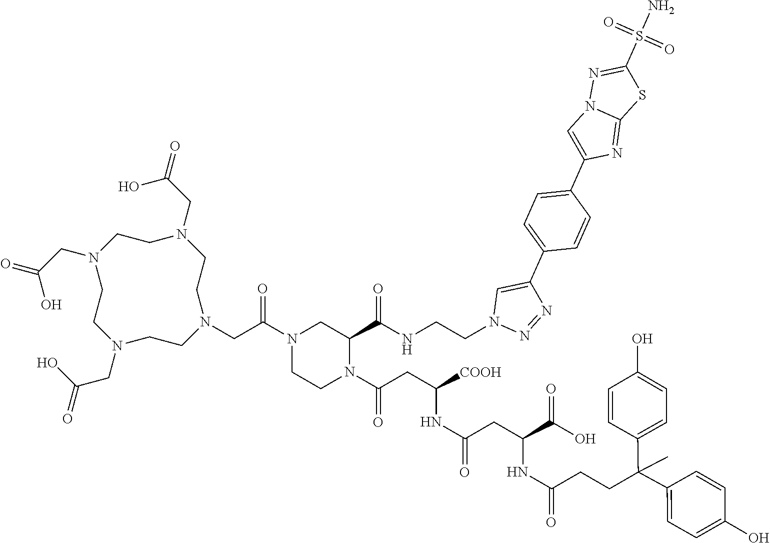 [SMILES: CC(CCC(=O)N[C@@H](CC(=O)N[C@@H](CC(=O)N1CCN(C(=O)CN2CCN(CC(=O)O)CCN(CC(=O)O)CCN(CC(=O)O)CC2)C[C@H]1C(=O)NCCn1cc(-c2ccc(-c3cn4nc(S(N)(=O)=O)sc4n3)cc2)nn1)C(=O)O)C(=O)O)(c1ccc(O)cc1)c1ccc(O)cc1] |
| 107.29 | 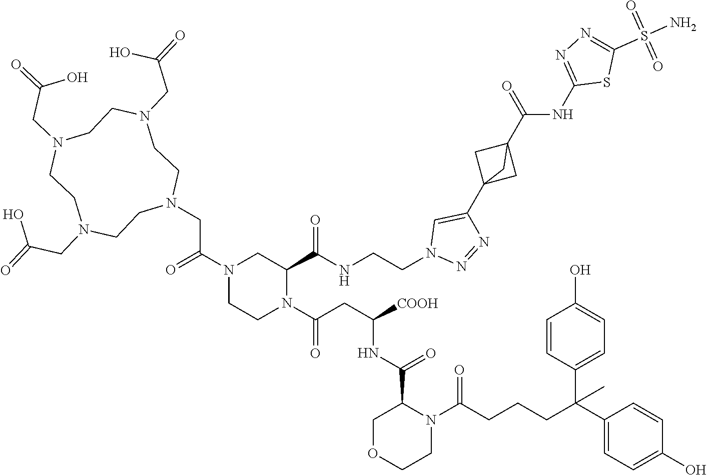 [SMILES: CC(CCCC(=O)N1CCOC[C@H]1C(=O)N[C@@H](CC(=O)N1CCN(C(=O)CN2CCN(CC(=O)O)CCN(CC(=O)O)CCN(CC(=O)O)CC2)C[C@H]1C(=O)NCCn1cc(C23CC(C(=O)Nc4nnc(S(N)(=O)=O)s4)(C2)C3)nn1)C(=O)O)(c1ccc(O)cc1)c1ccc(O)cc1] |
| 107.30 | 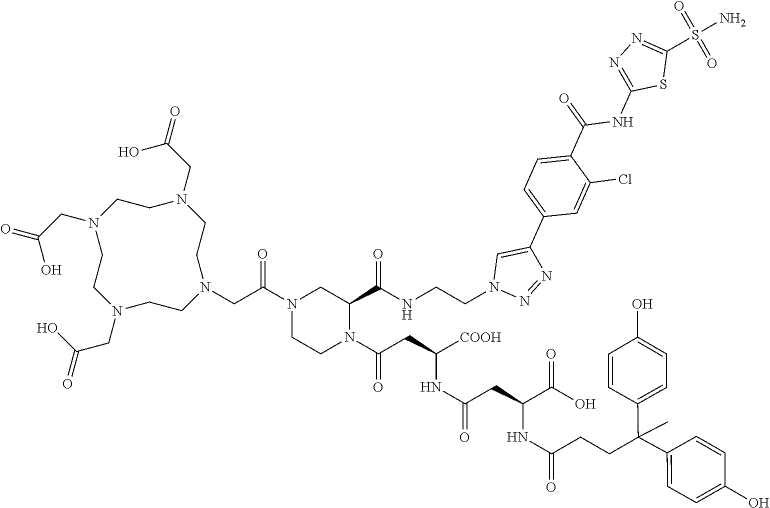 [SMILES: CC(CCC(=O)N[C@@H](CC(=O)N[C@@H](CC(=O)N1CCN(C(=O)CN2CCN(CC(=O)O)CCN(CC(=O)O)CCN(CC(=O)O)CC2)C[C@H]1C(=O)NCCn1cc(-c2ccc(C(=O)Nc3nnc(S(N)(=O)=O)s3)c(Cl)c2)nn1)C(=O)O)C(=O)O)(c1ccc(O)cc1)c1ccc(O)cc1] |
| 107.31 | 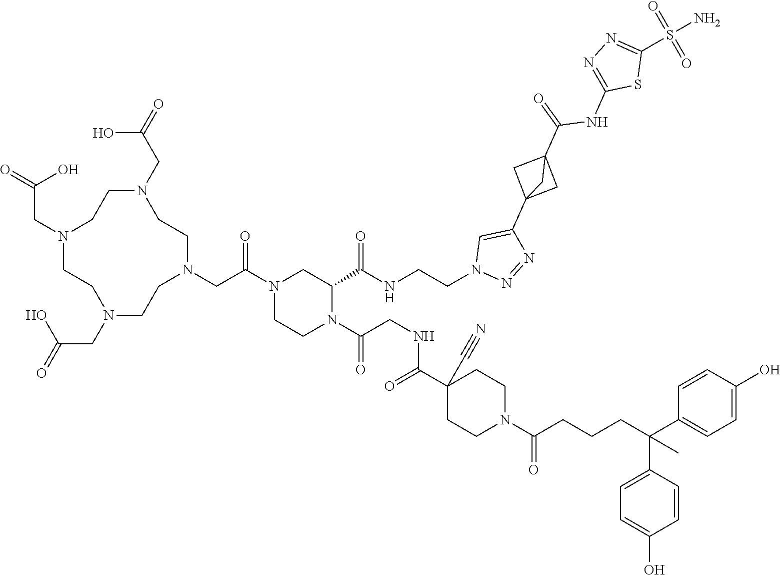 [SMILES: CC(CCCC(=O)N1CCC(C#N)(C(=O)NCC(=O)N2CCN(C(=O)CN3CCN(CC(=O)O)CCN(CC(=O)O)CCN(CC(=O)O)CC3)C[C@@H]2C(=O)NCCn2cc(C34CC(C(=O)Nc5nnc(S(N)(=O)=O)s5)(C3)C4)nn2)CC1)(c1ccc(O)cc1)c1ccc(O)cc1] |
| 107.32 | 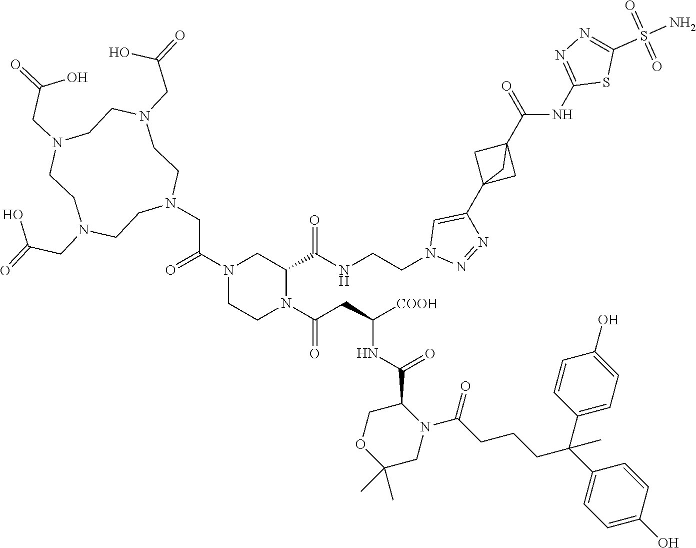 [SMILES: CC1(C)CN(C(=O)CCCC(C)(c2ccc(O)cc2)c2ccc(O)cc2)[C@H](C(=O)N[C@@H](CC(=O)N2CCN(C(=O)CN3CCN(CC(=O)O)CCN(CC(=O)O)CCN(CC(=O)O)CC3)C[C@@H]2C(=O)NCCn2cc(C34CC(C(=O)Nc5nnc(S(N)(=O)=O)s5)(C3)C4)nn2)C(=O)O)CO1] |
| 107.33 | 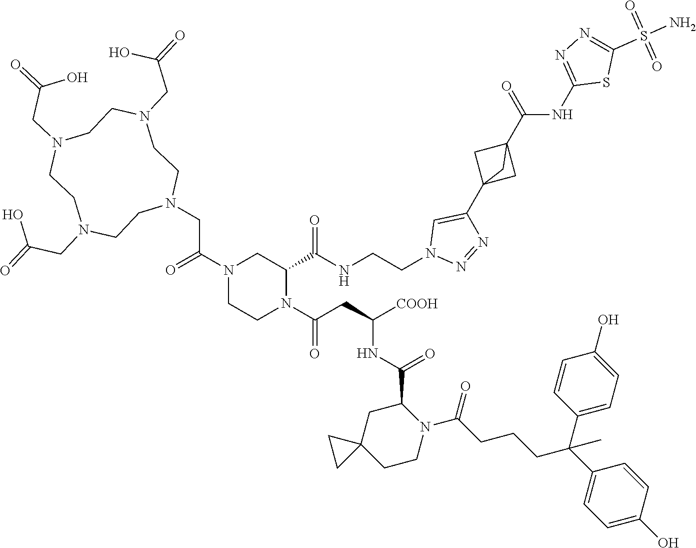 [SMILES: CC(CCCC(=O)N1CCC2(CC2)C[C@H]1C(=O)N[C@@H](CC(=O)N1CCN(C(=O)CN2CCN(CC(=O)O)CCN(CC(=O)O)CCN(CC(=O)O)CC2)C[C@@H]1C(=O)NCCn1cc(C23CC(C(=O)Nc4nnc(S(N)(=O)=O)s4)(C2)C3)nn1)C(=O)O)(c1ccc(O)cc1)c1ccc(O)cc1] |
| 107.34 | 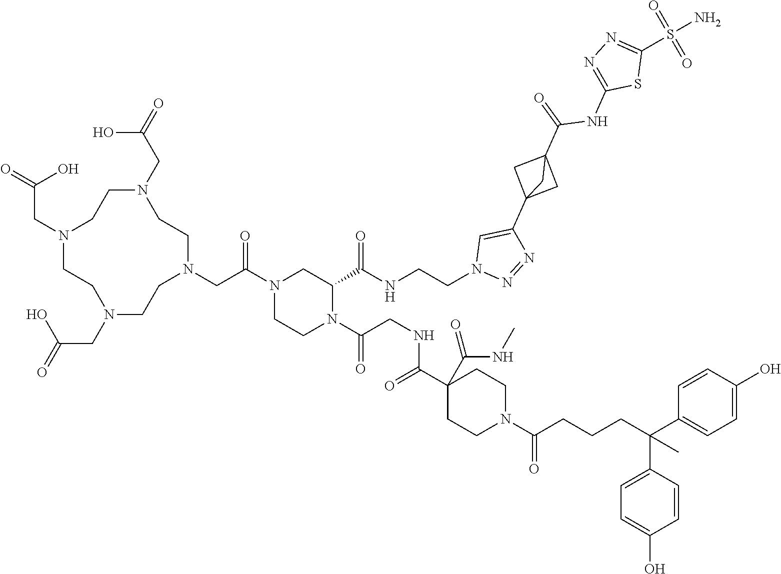 [SMILES: CNC(=O)C1(C(=O)NCC(=O)N2CCN(C(=O)CN3CCN(CC(=O)O)CCN(CC(=O)O)CCN(CC(=O)O)CC3)C[C@@H]2C(=O)NCCn2cc(C34CC(C(=O)Nc5nnc(S(N)(=O)=O)s5)(C3)C4)nn2)CCN(C(=O)CCCC(C)(c2ccc(O)cc2)c2ccc(O)cc2)CC1] |
| 107.35 | 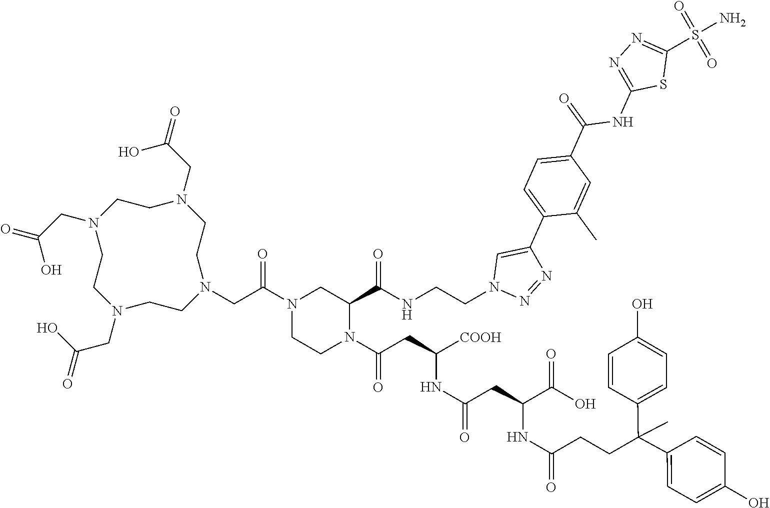 [SMILES: Cc1cc(C(=O)Nc2nnc(S(N)(=O)=O)s2)ccc1-c1cn(CCNC(=O)[C@@H]2CN(C(=O)CN3CCN(CC(=O)O)CCN(CC(=O)O)CCN(CC(=O)O)CC3)CCN2C(=O)C[C@H](NC(=O)C[C@H](NC(=O)CCC(C)(c2ccc(O)cc2)c2ccc(O)cc2)C(=O)O)C(=O)O)nn1] |
| 107.36 | 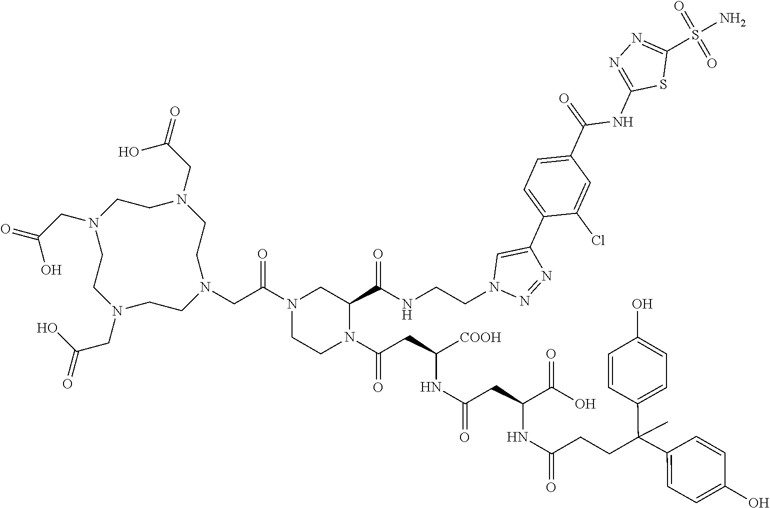 [SMILES: CC(CCC(=O)N[C@@H](CC(=O)N[C@@H](CC(=O)N1CCN(C(=O)CN2CCN(CC(=O)O)CCN(CC(=O)O)CCN(CC(=O)O)CC2)C[C@H]1C(=O)NCCn1cc(-c2ccc(C(=O)Nc3nnc(S(N)(=O)=O)s3)cc2Cl)nn1)C(=O)O)C(=O)O)(c1ccc(O)cc1)c1ccc(O)cc1] |
| 107.37 | 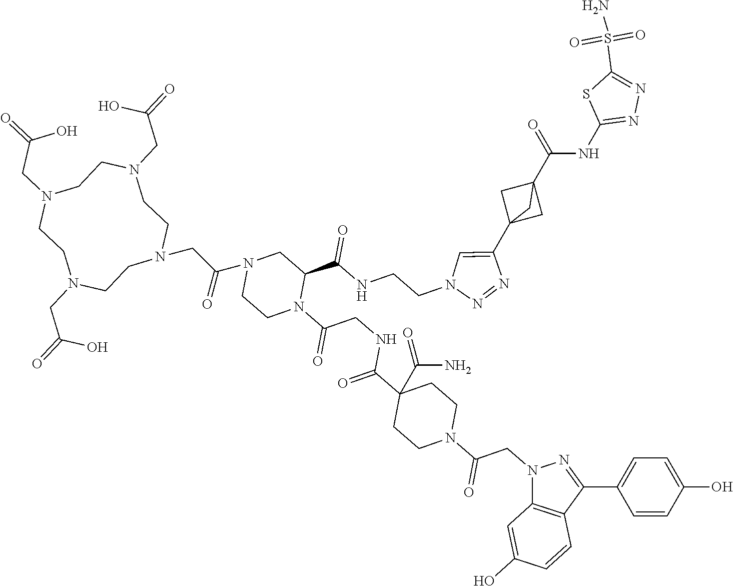 [SMILES: NC(=O)C1(C(=O)NCC(=O)N2CCN(C(=O)CN3CCN(CC(=O)O)CCN(CC(=O)O)CCN(CC(=O)O)CC3)C[C@H]2C(=O)NCCn2cc(C34CC(C(=O)Nc5nnc(S(N)(=O)=O)s5)(C3)C4)nn2)CCN(C(=O)Cn2nc(-c3ccc(O)cc3)c3ccc(O)cc32)CC1] |
| 107.38 | 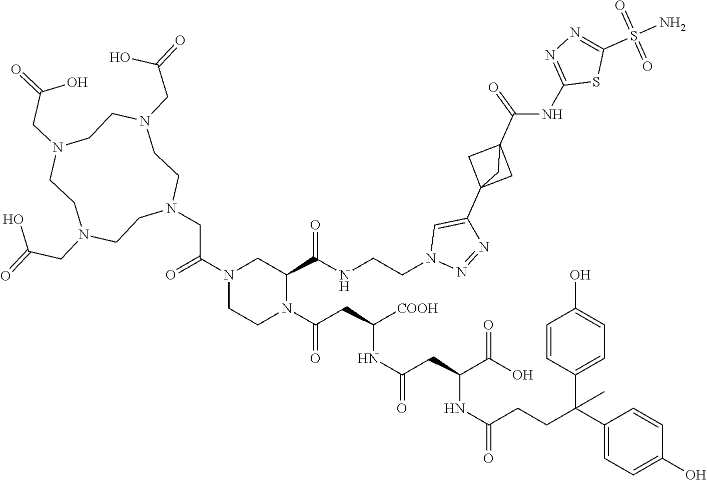 [SMILES: CC(CCC(=O)N[C@@H](CC(=O)N[C@@H](CC(=O)N1CCN(C(=O)CN2CCN(CC(=O)O)CCN(CC(=O)O)CCN(CC(=O)O)CC2)C[C@H]1C(=O)NCCn1cc(C23CC(C(=O)Nc4nnc(S(N)(=O)=O)s4)(C2)C3)nn1)C(=O)O)C(=O)O)(c1ccc(O)cc1)c1ccc(O)cc1] |
| 107.39 | 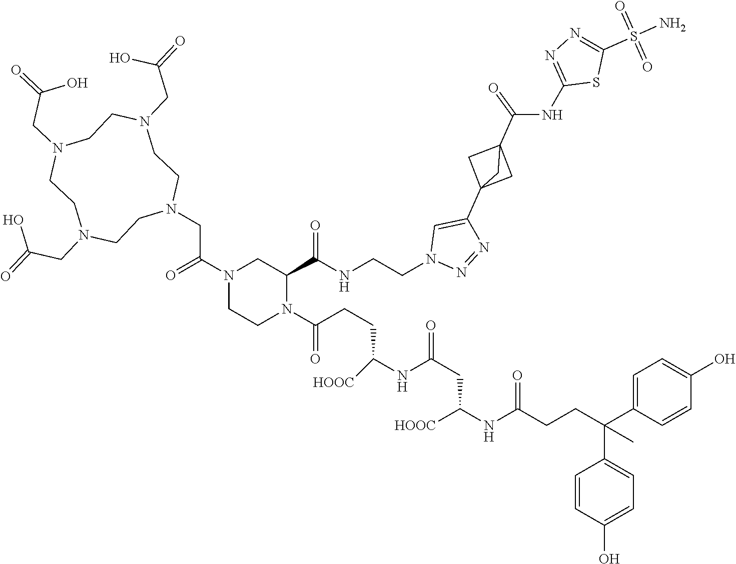 [SMILES: CC(CCC(=O)N[C@@H](CC(=O)N[C@@H](CCC(=O)N1CCN(C(=O)CN2CCN(CC(=O)O)CCN(CC(=O)O)CCN(CC(=O)O)CC2)C[C@H]1C(=O)NCCn1cc(C23CC(C(=O)Nc4nnc(S(N)(=O)=O)s4)(C2)C3)nn1)C(=O)O)C(=O)O)(c1ccc(O)cc1)c1ccc(O)cc1] |
| 107.40 | 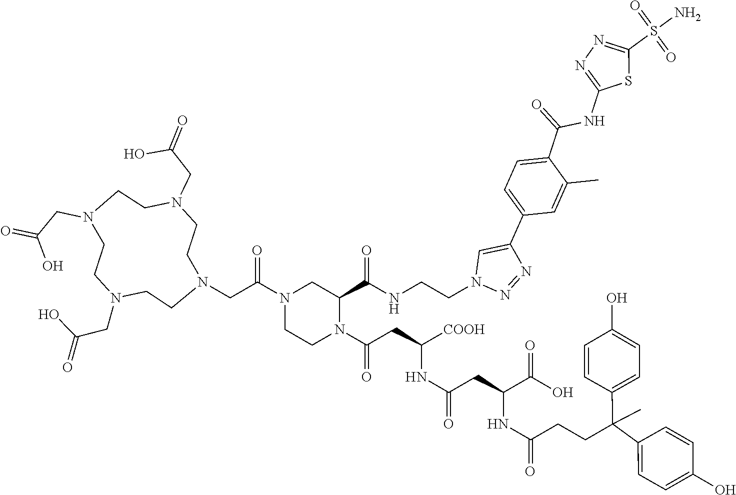 [SMILES: Cc1cc(-c2cn(CCNC(=O)[C@@H]3CN(C(=O)CN4CCN(CC(=O)O)CCN(CC(=O)O)CCN(CC(=O)O)CC4)CCN3C(=O)C[C@H](NC(=O)C[C@H](NC(=O)CCC(C)(c3ccc(O)cc3)c3ccc(O)cc3)C(=O)O)C(=O)O)nn2)ccc1C(=O)Nc1nnc(S(N)(=O)=O)s1] |
| 107.41 | 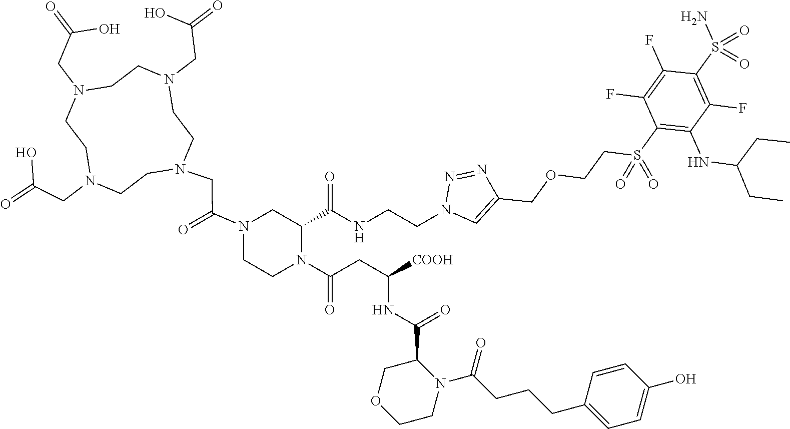 [SMILES: CCC(CC)Nc1c(F)c(S(N)(=O)=O)c(F)c(F)c1S(=O)(=O)CCOCc1cn(CCNC(=O)[C@H]2CN(C(=O)CN3CCN(CC(=O)O)CCN(CC(=O)O)CCN(CC(=O)O)CC3)CCN2C(=O)C[C@H](NC(=O)[C@@H]2COCCN2C(=O)CCCc2ccc(O)cc2)C(=O)O)nn1] |
| 107.42 | 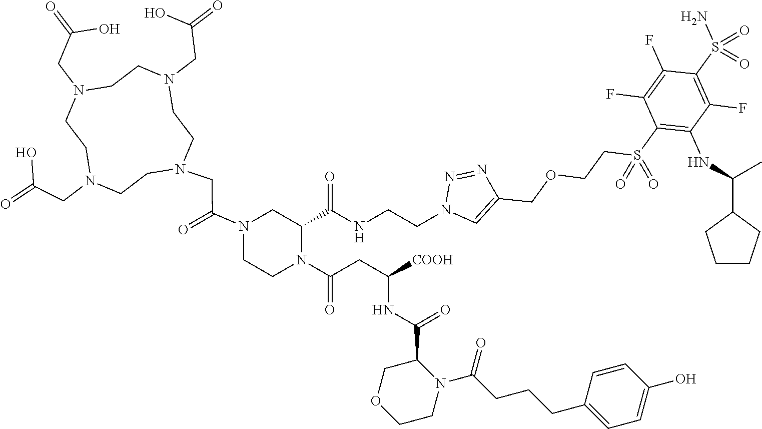 [SMILES: C[C@H](Nc1c(F)c(S(N)(=O)=O)c(F)c(F)c1S(=O)(=O)CCOCc1cn(CCNC(=O)[C@H]2CN(C(=O)CN3CCN(CC(=O)O)CCN(CC(=O)O)CCN(CC(=O)O)CC3)CCN2C(=O)C[C@H](NC(=O)[C@@H]2COCCN2C(=O)CCCc2ccc(O)cc2)C(=O)O)nn1)C1CCCC1] |
| 107.43 | 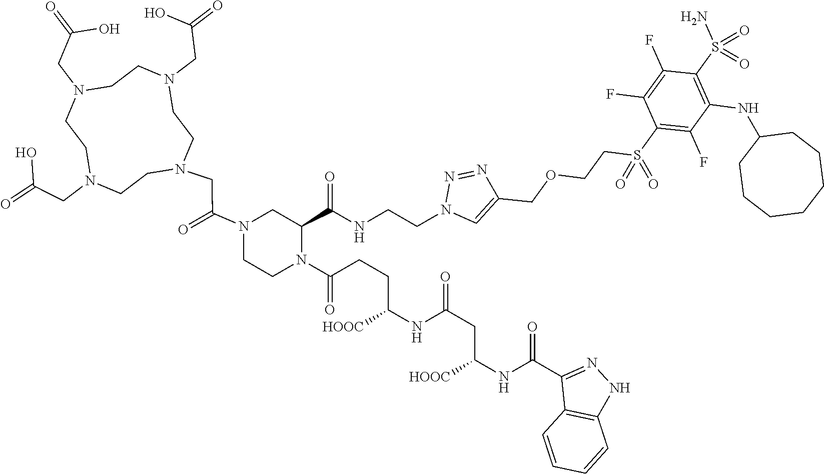 [SMILES: NS(=O)(=O)c1c(F)c(F)c(S(=O)(=O)CCOCc2cn(CCNC(=O)[C@@H]3CN(C(=O)CN4CCN(CC(=O)O)CCN(CC(=O)O)CCN(CC(=O)O)CC4)CCN3C(=O)CC[C@H](NC(=O)C[C@H](NC(=O)c3n[nH]c4ccccc34)C(=O)O)C(=O)O)nn2)c(F)c1NC1CCCCCCC1] |
| 107.44 | 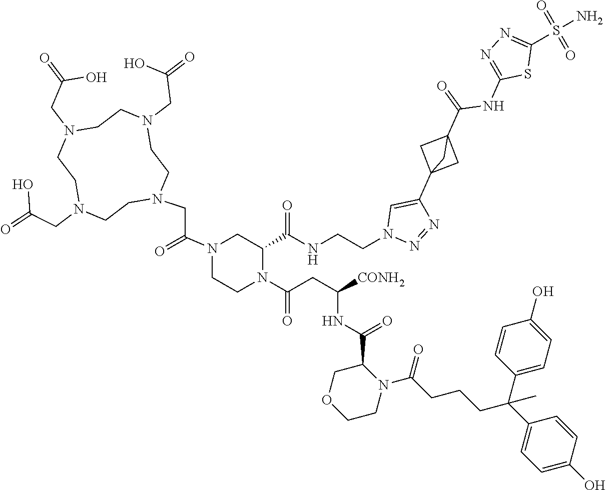 [SMILES: CC(CCCC(=O)N1CCOC[C@H]1C(=O)N[C@@H](CC(=O)N1CCN(C(=O)CN2CCN(CC(=O)O)CCN(CC(=O)O)CCN(CC(=O)O)CC2)C[C@@H]1C(=O)NCCn1cc(C23CC(C(=O)Nc4nnc(S(N)(=O)=O)s4)(C2)C3)nn1)C(N)=O)(c1ccc(O)cc1)c1ccc(O)cc1] |
| 107.45 | 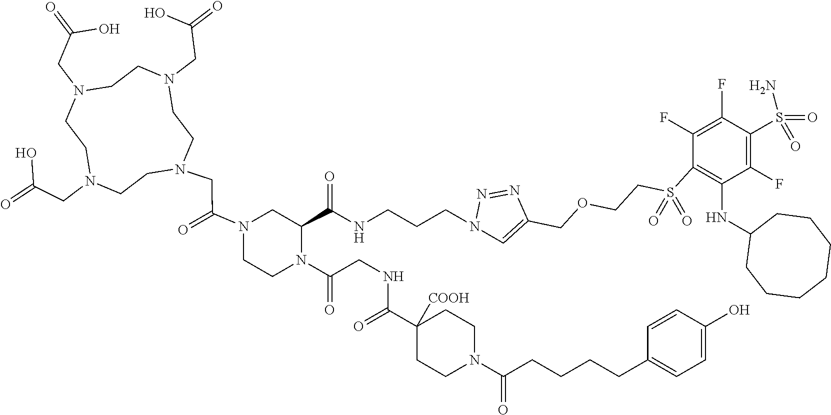 [SMILES: NS(=O)(=O)c1c(F)c(F)c(S(=O)(=O)CCOCc2cn(CCCNC(=O)[C@@H]3CN(C(=O)CN4CCN(CC(=O)O)CCN(CC(=O)O)CCN(CC(=O)O)CC4)CCN3C(=O)CNC(=O)C3(C(=O)O)CCN(C(=O)CCCCc4ccc(O)cc4)CC3)nn2)c(NC2CCCCCCC2)c1F] |
| 107.46 | 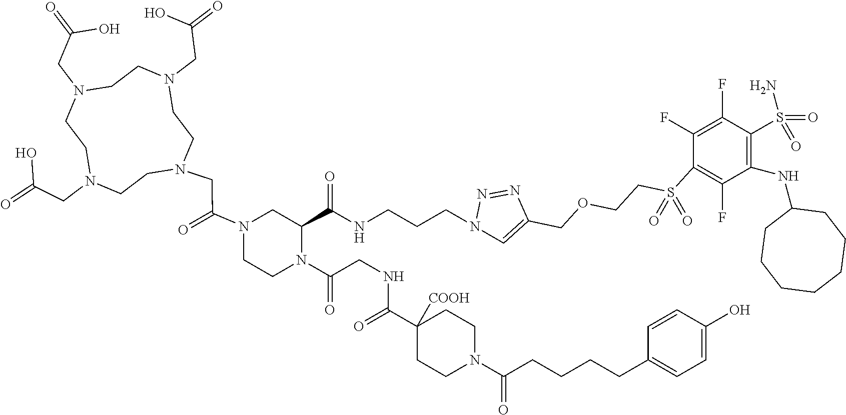 [SMILES: NS(=O)(=O)c1c(F)c(F)c(S(=O)(=O)CCOCc2cn(CCCNC(=O)[C@@H]3CN(C(=O)CN4CCN(CC(=O)O)CCN(CC(=O)O)CCN(CC(=O)O)CC4)CCN3C(=O)CNC(=O)C3(C(=O)O)CCN(C(=O)CCCCc4ccc(O)cc4)CC3)nn2)c(F)c1NC1CCCCCCC1] |
| 107.47 | 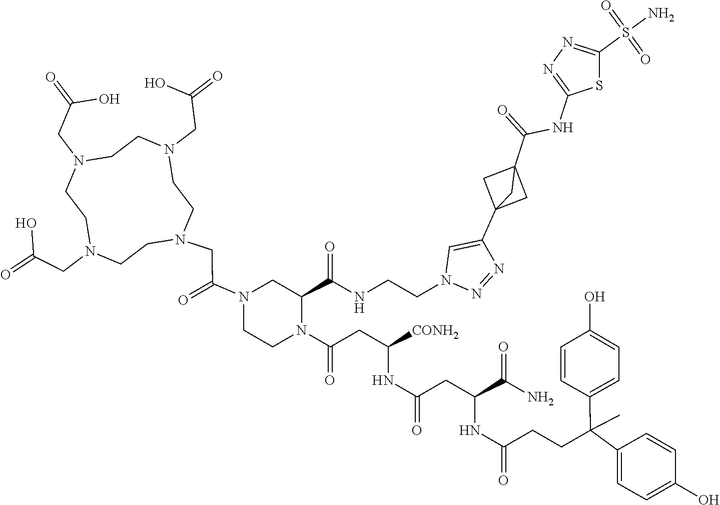 [SMILES: CC(CCC(=O)N[C@@H](CC(=O)N[C@@H](CC(=O)N1CCN(C(=O)CN2CCN(CC(=O)O)CCN(CC(=O)O)CCN(CC(=O)O)CC2)C[C@H]1C(=O)NCCn1cc(C23CC(C(=O)Nc4nnc(S(N)(=O)=O)s4)(C2)C3)nn1)C(N)=O)C(N)=O)(c1ccc(O)cc1)c1ccc(O)cc1] |
| 107.48 | 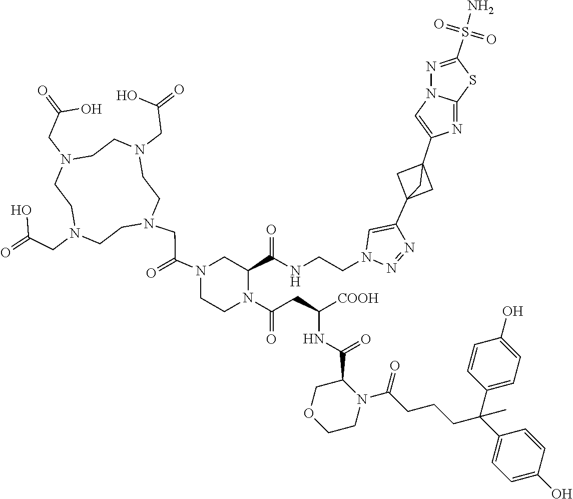 [SMILES: CC(CCCC(=O)N1CCOC[C@H]1C(=O)N[C@@H](CC(=O)N1CCN(C(=O)CN2CCN(CC(=O)O)CCN(CC(=O)O)CCN(CC(=O)O)CC2)C[C@H]1C(=O)NCCn1cc(C23CC(c4cn5nc(S(N)(=O)=O)sc5n4)(C2)C3)nn1)C(=O)O)(c1ccc(O)cc1)c1ccc(O)cc1] |
| 107.49 | 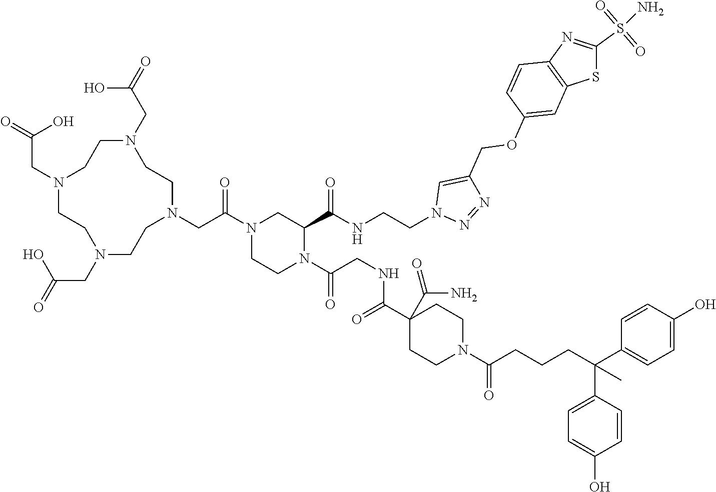 [SMILES: CC(CCCC(=O)N1CCC(C(N)=O)(C(=O)NCC(=O)N2CCN(C(=O)CN3CCN(CC(=O)O)CCN(CC(=O)O)CCN(CC(=O)O)CC3)C[C@H]2C(=O)NCCn2cc(COc3ccc4nc(S(N)(=O)=O)sc4c3)nn2)CC1)(c1ccc(O)cc1)c1ccc(O)cc1] |
| 107.50 | 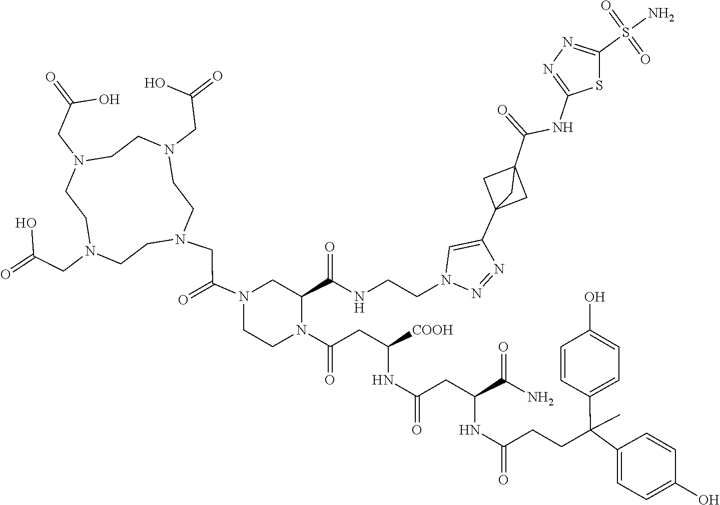 [SMILES: CC(CCC(=O)N[C@@H](CC(=O)N[C@@H](CC(=O)N1CCN(C(=O)CN2CCN(CC(=O)O)CCN(CC(=O)O)CCN(CC(=O)O)CC2)C[C@H]1C(=O)NCCn1cc(C23CC(C(=O)Nc4nnc(S(N)(=O)=O)s4)(C2)C3)nn1)C(=O)O)C(N)=O)(c1ccc(O)cc1)c1ccc(O)cc1] |
| 107.51 | 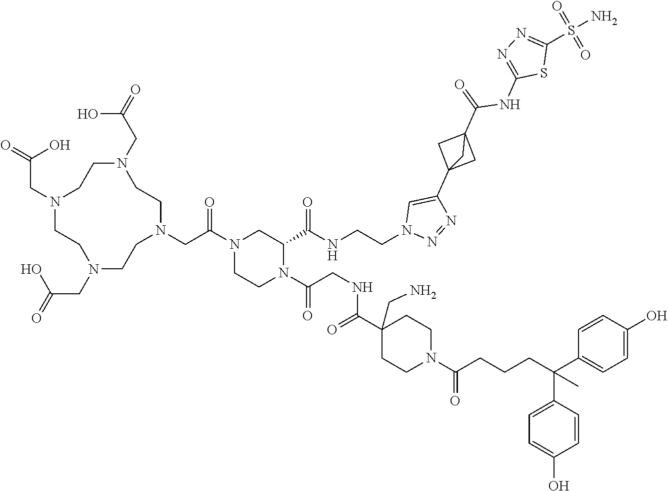 [SMILES: CC(CCCC(=O)N1CCC(CN)(C(=O)NCC(=O)N2CCN(C(=O)CN3CCN(CC(=O)O)CCN(CC(=O)O)CCN(CC(=O)O)CC3)C[C@@H]2C(=O)NCCn2cc(C34CC(C(=O)Nc5nnc(S(N)(=O)=O)s5)(C3)C4)nn2)CC1)(c1ccc(O)cc1)c1ccc(O)cc1] |
| 107.52 | 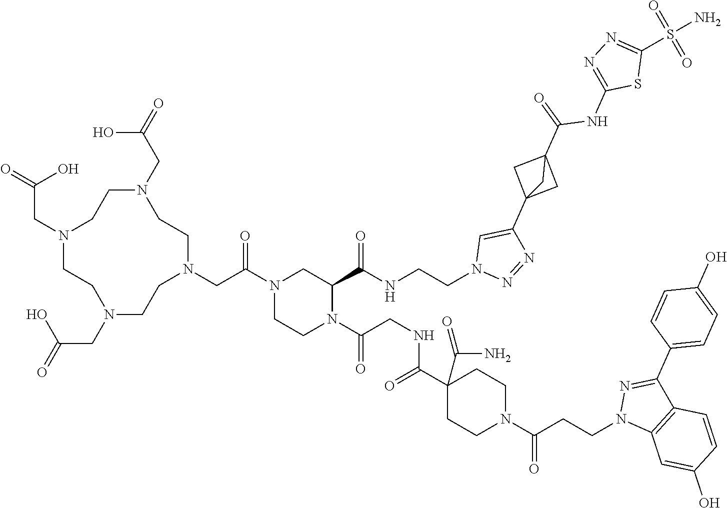 [SMILES: NC(=O)C1(C(=O)NCC(=O)N2CCN(C(=O)CN3CCN(CC(=O)O)CCN(CC(=O)O)CCN(CC(=O)O)CC3)C[C@H]2C(=O)NCCn2cc(C34CC(C(=O)Nc5nnc(S(N)(=O)=O)s5)(C3)C4)nn2)CCN(C(=O)CCn2nc(-c3ccc(O)cc3)c3ccc(O)cc32)CC1] |
| 107.53 | 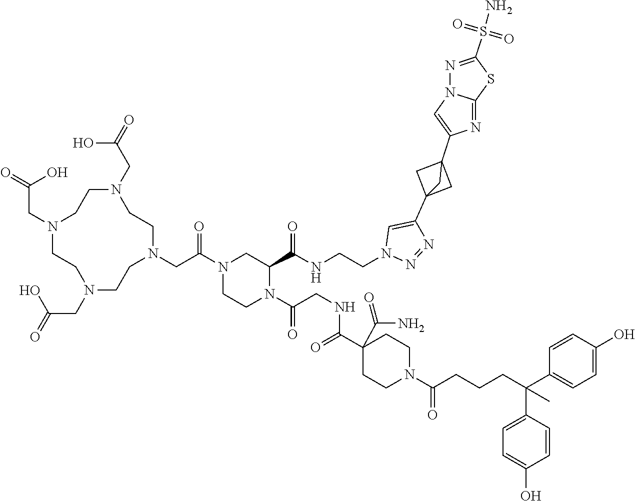 [SMILES: CC(CCCC(=O)N1CCC(C(N)=O)(C(=O)NCC(=O)N2CCN(C(=O)CN3CCN(CC(=O)O)CCN(CC(=O)O)CCN(CC(=O)O)CC3)C[C@H]2C(=O)NCCn2cc(C34CC(c5cn6nc(S(N)(=O)=O)sc6n5)(C3)C4)nn2)CC1)(c1ccc(O)cc1)c1ccc(O)cc1] |
| 107.54 | 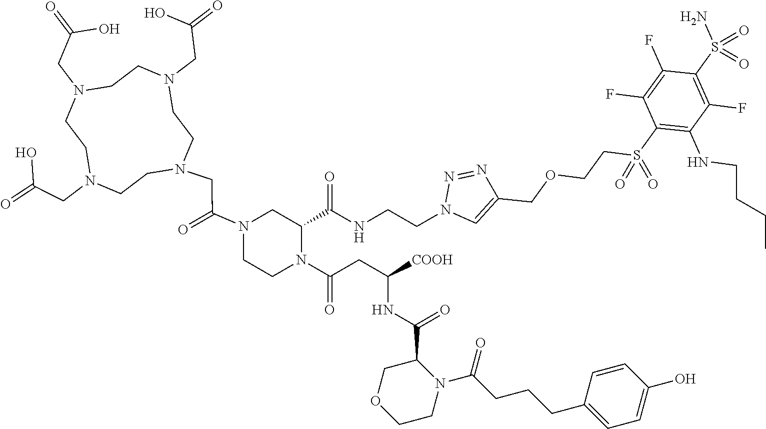 [SMILES: CCCCNc1c(F)c(S(N)(=O)=O)c(F)c(F)c1S(=O)(=O)CCOCc1cn(CCNC(=O)[C@H]2CN(C(=O)CN3CCN(CC(=O)O)CCN(CC(=O)O)CCN(CC(=O)O)CC3)CCN2C(=O)C[C@H](NC(=O)[C@@H]2COCCN2C(=O)CCCc2ccc(O)cc2)C(=O)O)nn1] |
| 107.55 | 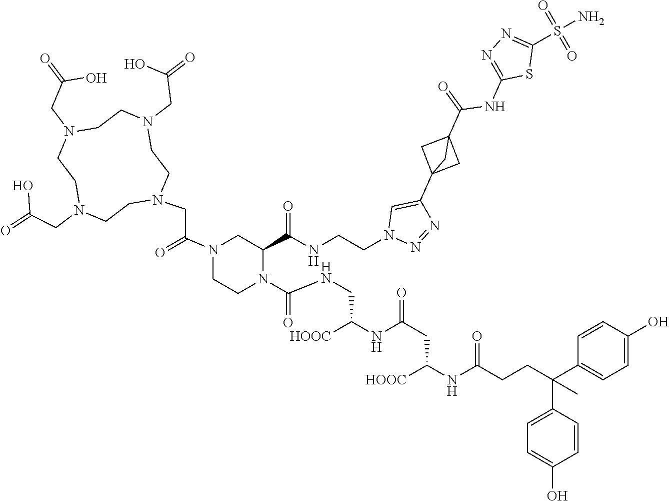 [SMILES: CC(CCC(=O)N[C@@H](CC(=O)N[C@@H](CNC(=O)N1CCN(C(=O)CN2CCN(CC(=O)O)CCN(CC(=O)O)CCN(CC(=O)O)CC2)C[C@H]1C(=O)NCCn1cc(C23CC(C(=O)Nc4nnc(S(N)(=O)=O)s4)(C2)C3)nn1)C(=O)O)C(=O)O)(c1ccc(O)cc1)c1ccc(O)cc1] |
| 107.56 | 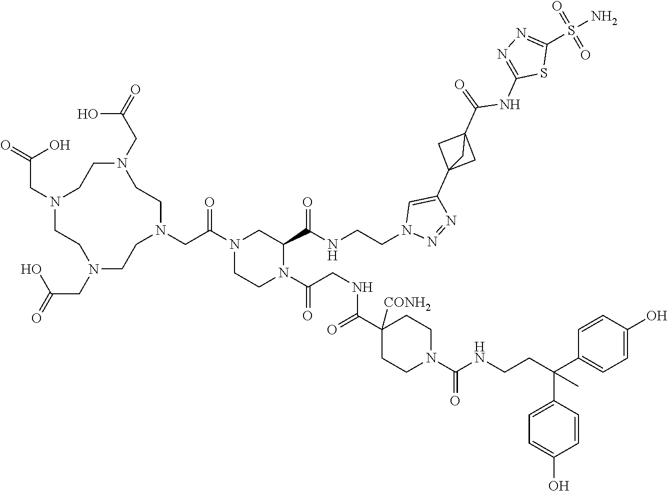 [SMILES: CC(CCNC(=O)N1CCC(C(N)=O)(C(=O)NCC(=O)N2CCN(C(=O)CN3CCN(CC(=O)O)CCN(CC(=O)O)CCN(CC(=O)O)CC3)C[C@H]2C(=O)NCCn2cc(C34CC(C(=O)Nc5nnc(S(N)(=O)=O)s5)(C3)C4)nn2)CC1)(c1ccc(O)cc1)c1ccc(O)cc1] |
| 107.57 | 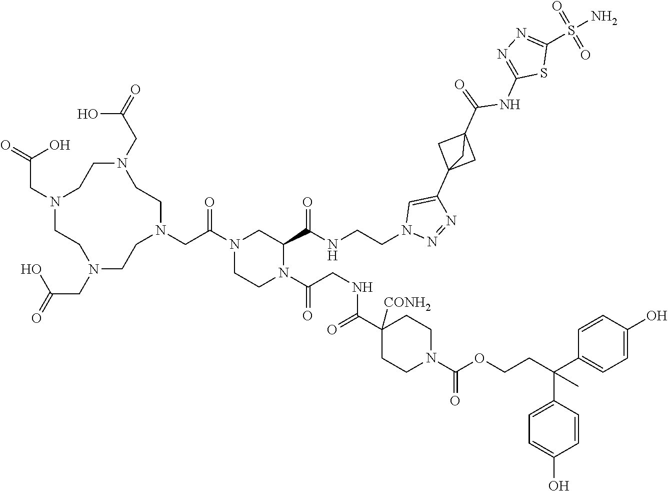 [SMILES: CC(CCOC(=O)N1CCC(C(N)=O)(C(=O)NCC(=O)N2CCN(C(=O)CN3CCN(CC(=O)O)CCN(CC(=O)O)CCN(CC(=O)O)CC3)C[C@H]2C(=O)NCCn2cc(C34CC(C(=O)Nc5nnc(S(N)(=O)=O)s5)(C3)C4)nn2)CC1)(c1ccc(O)cc1)c1ccc(O)cc1] |
| 107.58 | 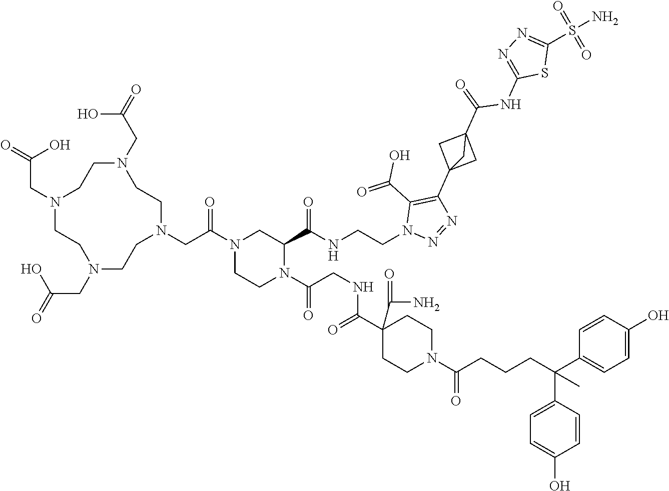 [SMILES: CC(CCCC(=O)N1CCC(C(N)=O)(C(=O)NCC(=O)N2CCN(C(=O)CN3CCN(CC(=O)O)CCN(CC(=O)O)CCN(CC(=O)O)CC3)C[C@H]2C(=O)NCCn2nnc(C34CC(C(=O)Nc5nnc(S(N)(=O)=O)s5)(C3)C4)c2C(=O)O)CC1)(c1ccc(O)cc1)c1ccc(O)cc1] |
| 107.59 | 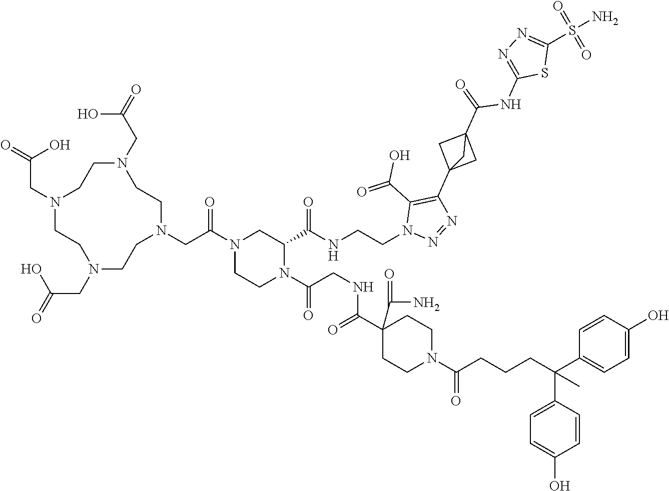 [SMILES: CC(CCCC(=O)N1CCC(C(N)=O)(C(=O)NCC(=O)N2CCN(C(=O)CN3CCN(CC(=O)O)CCN(CC(=O)O)CCN(CC(=O)O)CC3)C[C@@H]2C(=O)NCCn2nnc(C34CC(C(=O)Nc5nnc(S(N)(=O)=O)s5)(C3)C4)c2C(=O)O)CC1)(c1ccc(O)cc1)c1ccc(O)cc1] |
| 107.60 | 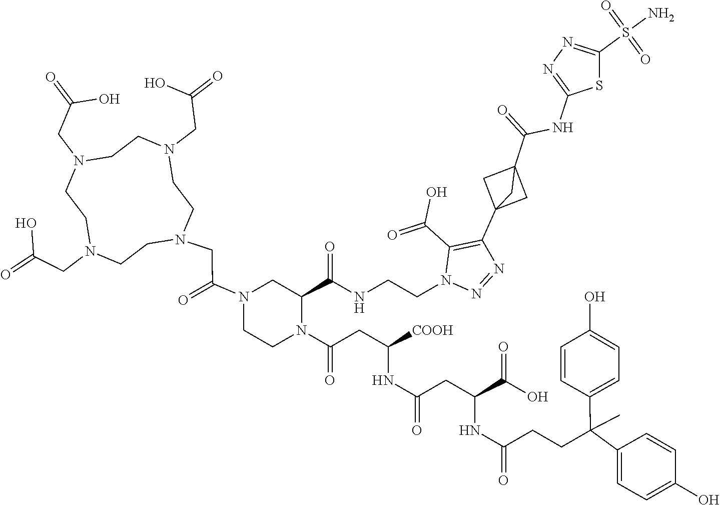 [SMILES: CC(CCC(=O)N[C@@H](CC(=O)N[C@@H](CC(=O)N1CCN(C(=O)CN2CCN(CC(=O)O)CCN(CC(=O)O)CCN(CC(=O)O)CC2)C[C@H]1C(=O)NCCn1nnc(C23CC(C(=O)Nc4nnc(S(N)(=O)=O)s4)(C2)C3)c1C(=O)O)C(=O)O)C(=O)O)(c1ccc(O)cc1)c1ccc(O)cc1] |
| 107.61 | 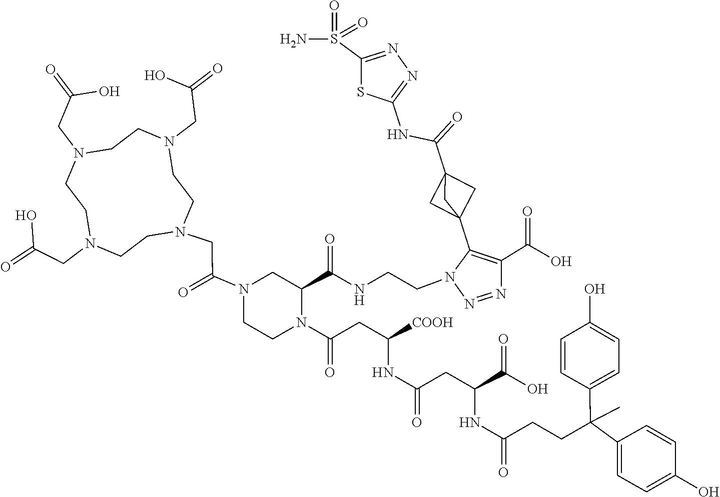 [SMILES: CC(CCC(=O)N[C@@H](CC(=O)N[C@@H](CC(=O)N1CCN(C(=O)CN2CCN(CC(=O)O)CCN(CC(=O)O)CCN(CC(=O)O)CC2)C[C@H]1C(=O)NCCn1nnc(C(=O)O)c1C12CC(C(=O)Nc3nnc(S(N)(=O)=O)s3)(C1)C2)C(=O)O)C(=O)O)(c1ccc(O)cc1)c1ccc(O)cc1] |
| 108 | 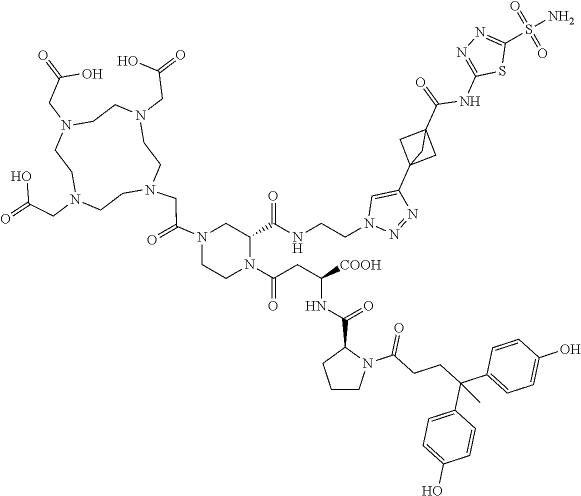 [SMILES: CC(CCC(=O)N1CCC[C@H]1C(=O)N[C@@H](CC(=O)N1CCN(C(=O)CN2CCN(CC(=O)O)CCN(CC(=O)O)CCN(CC(=O)O)CC2)C[C@@H]1C(=O)NCCn1cc(C23CC(C(=O)Nc4nnc(S(N)(=O)=O)s4)(C2)C3)nn1)C(=O)O)(c1ccc(O)cc1)c1ccc(O)cc1] |
| 108.1 | 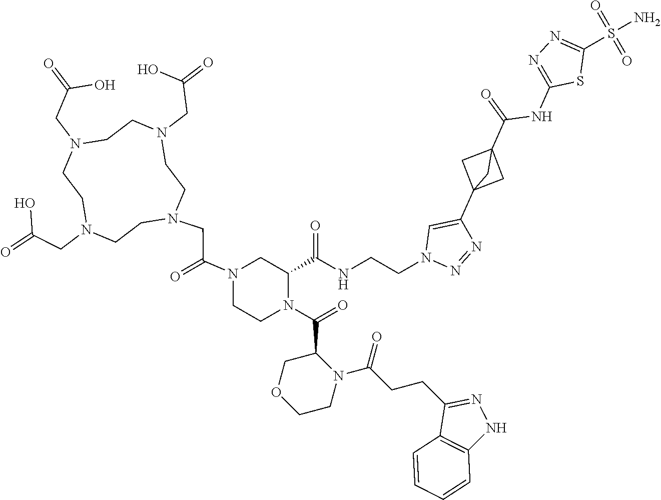 [SMILES: NS(=O)(=O)c1nnc(NC(=O)C23CC(c4cn(CCNC(=O)[C@H]5CN(C(=O)CN6CCN(CC(=O)O)CCN(CC(=O)O)CCN(CC(=O)O)CC6)CCN5C(=O)[C@@H]5COCCN5C(=O)CCc5n[nH]c6ccccc56)nn4)(C2)C3)s1] |
| 108.2 | 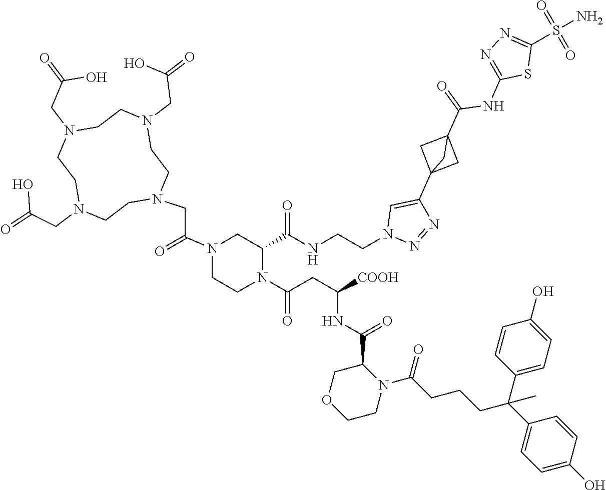 [SMILES: CC(CCCC(=O)N1CCOC[C@H]1C(=O)N[C@@H](CC(=O)N1CCN(C(=O)CN2CCN(CC(=O)O)CCN(CC(=O)O)CCN(CC(=O)O)CC2)C[C@@H]1C(=O)NCCn1cc(C23CC(C(=O)Nc4nnc(S(N)(=O)=O)s4)(C2)C3)nn1)C(=O)O)(c1ccc(O)cc1)c1ccc(O)cc1] |
| 108.3 | 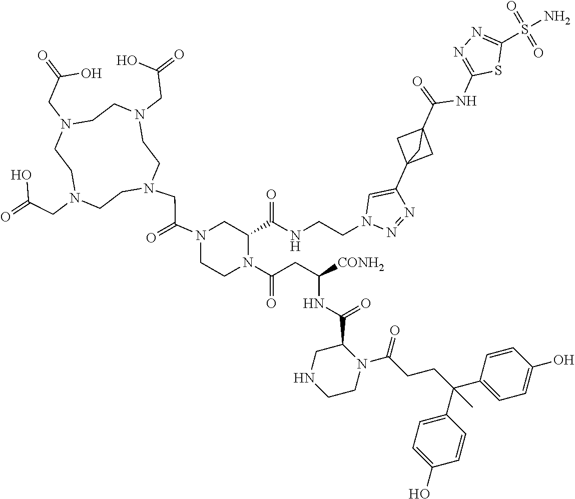 [SMILES: CC(CCC(=O)N1CCNC[C@H]1C(=O)N[C@@H](CC(=O)N1CCN(C(=O)CN2CCN(CC(=O)O)CCN(CC(=O)O)CCN(CC(=O)O)CC2)C[C@@H]1C(=O)NCCn1cc(C23CC(C(=O)Nc4nnc(S(N)(=O)=O)s4)(C2)C3)nn1)C(N)=O)(c1ccc(O)cc1)c1ccc(O)cc1] |
| 108.4 | 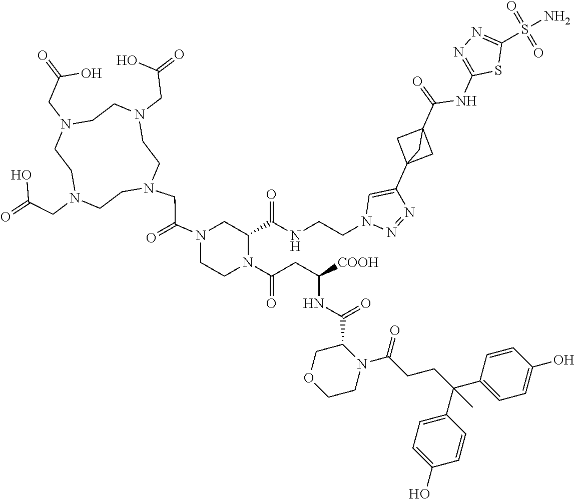 [SMILES: CC(CCC(=O)N1CCOC[C@@H]1C(=O)N[C@@H](CC(=O)N1CCN(C(=O)CN2CCN(CC(=O)O)CCN(CC(=O)O)CCN(CC(=O)O)CC2)C[C@@H]1C(=O)NCCn1cc(C23CC(C(=O)Nc4nnc(S(N)(=O)=O)s4)(C2)C3)nn1)C(=O)O)(c1ccc(O)cc1)c1ccc(O)cc1] |
| 108.5 | 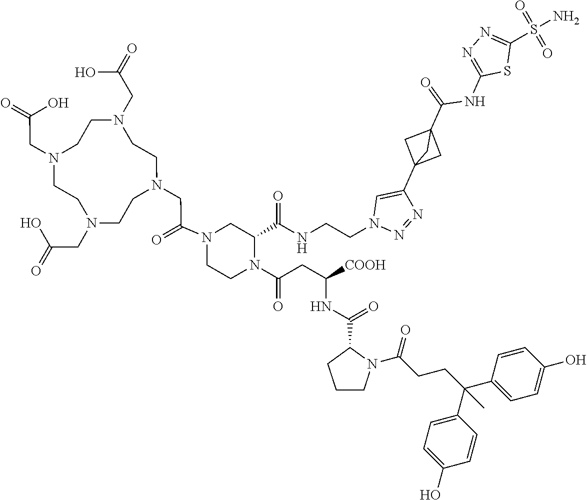 [SMILES: CC(CCC(=O)N1CCC[C@@H]1C(=O)N[C@@H](CC(=O)N1CCN(C(=O)CN2CCN(CC(=O)O)CCN(CC(=O)O)CCN(CC(=O)O)CC2)C[C@@H]1C(=O)NCCn1cc(C23CC(C(=O)Nc4nnc(S(N)(=O)=O)s4)(C2)C3)nn1)C(=O)O)(c1ccc(O)cc1)c1ccc(O)cc1] |
| 109 | 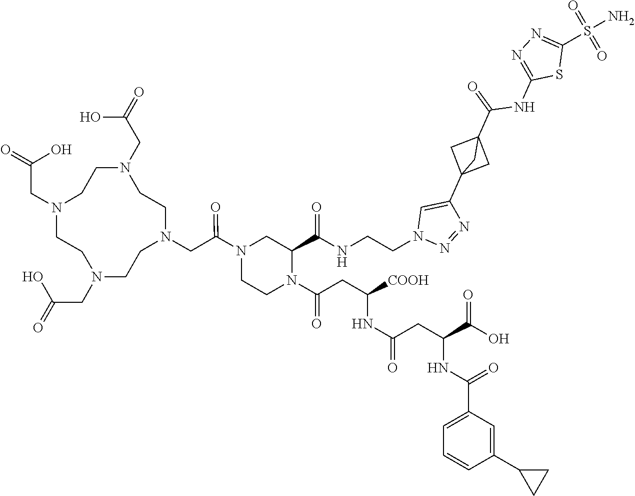 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)C[C@H](NC(=O)C[C@H](NC(=O)c5cccc(C6CC6)c5)C(=O)O)C(=O)O)nn4)(C2)C3)s1] |
| 109.1 | 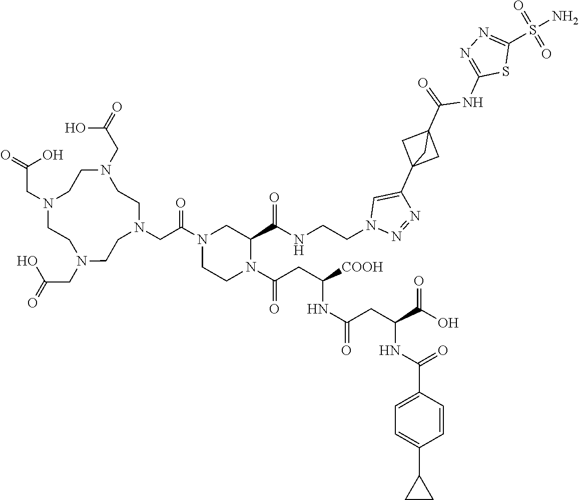 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)C[C@H](NC(=O)C[C@H](NC(=O)c5ccc(C6CC6)cc5)C(=O)O)C(=O)O)nn4)(C2)C3)s1] |
| 109.2 | 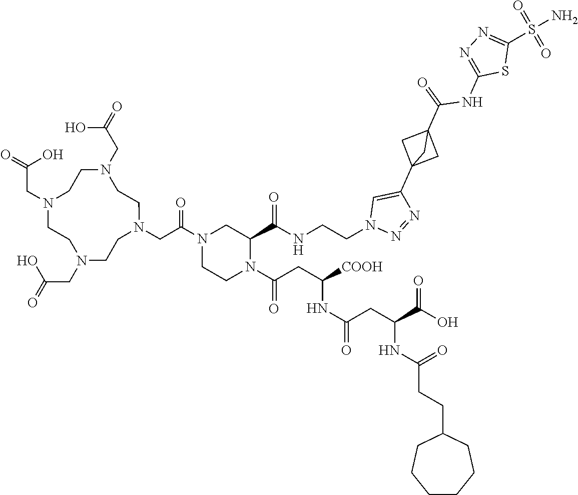 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)C[C@H](NC(=O)C[C@H](NC(=O)CCC5CCCCCC5)C(=O)O)C(=O)O)nn4)(C2)C3)s1] |
| 109.3 | 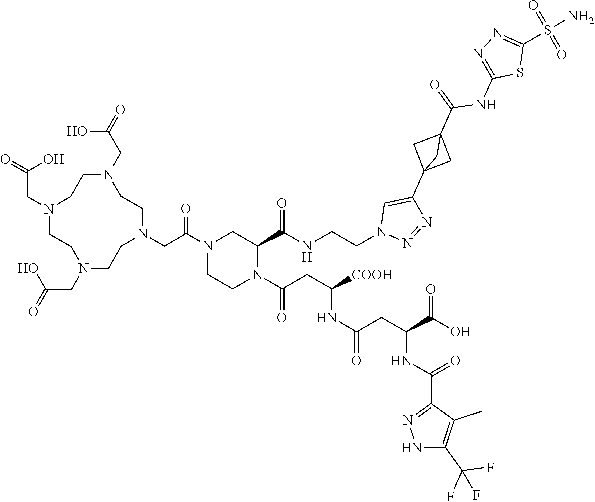 [SMILES: Cc1c(C(=O)N[C@@H](CC(=O)N[C@@H](CC(=O)N2CCN(C(=O)CN3CCN(CC(=O)O)CCN(CC(=O)O)CCN(CC(=O)O)CC3)C[C@H]2C(=O)NCCn2cc(C34CC(C(=O)Nc5nnc(S(N)(=O)=O)s5)(C3)C4)nn2)C(=O)O)C(=O)O)n[nH]c1C(F)(F)F] |
| 109.4 | 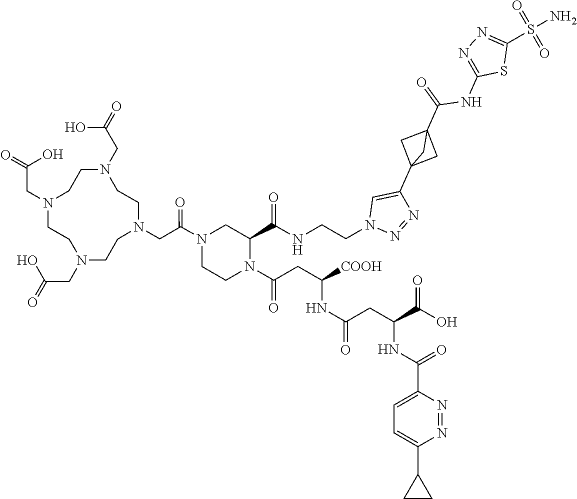 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)C[C@H](NC(=O)C[C@H](NC(=O)c5ccc(C6CC6)nn5)C(=O)O)C(=O)O)nn4)(C2)C3)s1] |
| 109.5 | 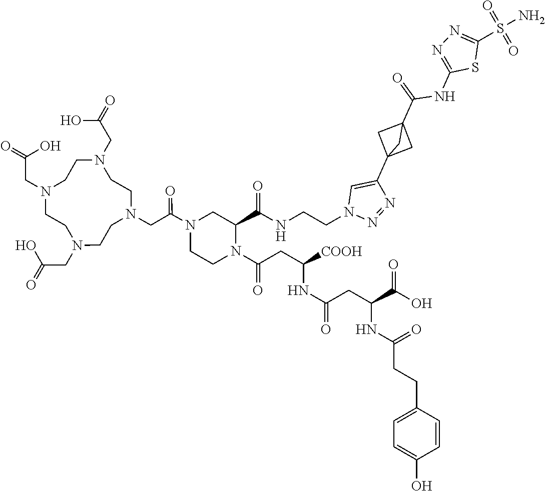 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)C[C@H](NC(=O)C[C@H](NC(=O)CCc5ccc(O)cc5)C(=O)O)C(=O)O)nn4)(C2)C3)s1] |
| 109.6 | 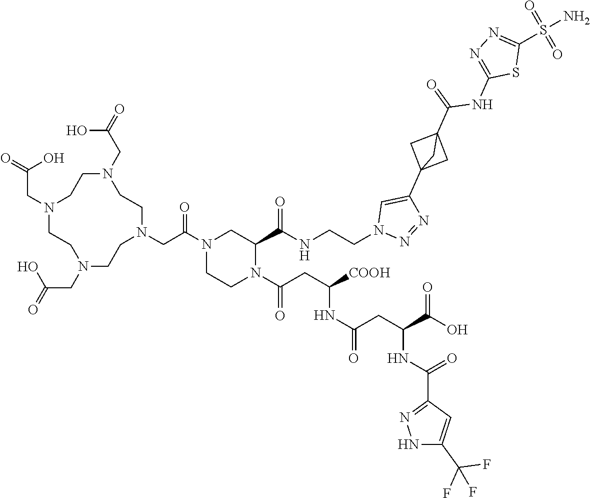 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)C[C@H](NC(=O)C[C@H](NC(=O)c5cc(C(F)(F)F)[nH]n5)C(=O)O)C(=O)O)nn4)(C2)C3)s1] |
| 109.7 | 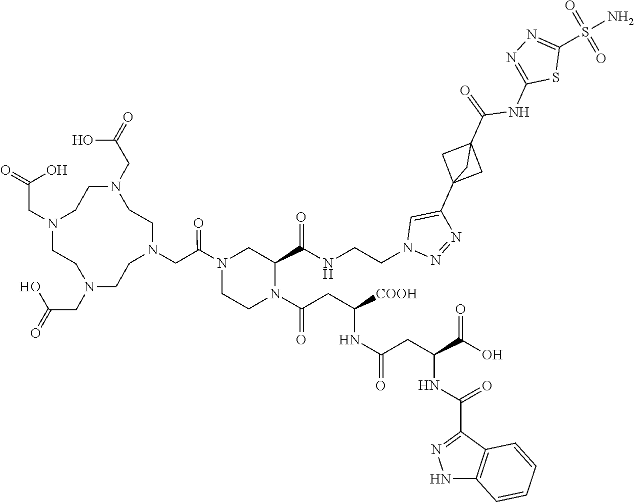 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)C[C@H](NC(=O)C[C@H](NC(=O)c5n[nH]c6ccccc56)C(=O)O)C(=O)O)nn4)(C2)C3)s1] |
| 109.8 | 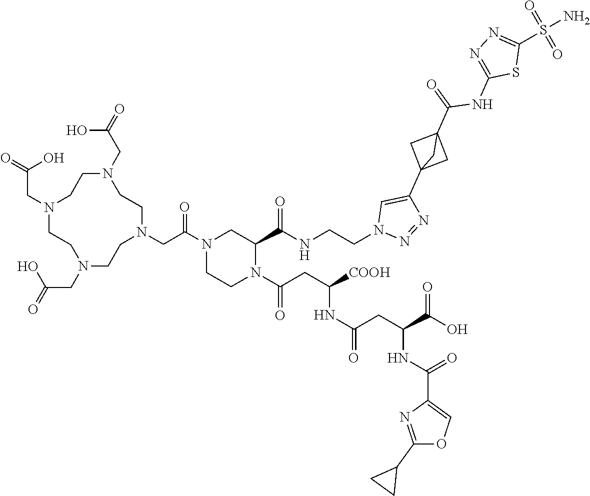 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)C[C@H](NC(=O)C[C@H](NC(=O)c5coc(C6CC6)n5)C(=O)O)C(=O)O)nn4)(C2)C3)s1] |
| 109.9 | 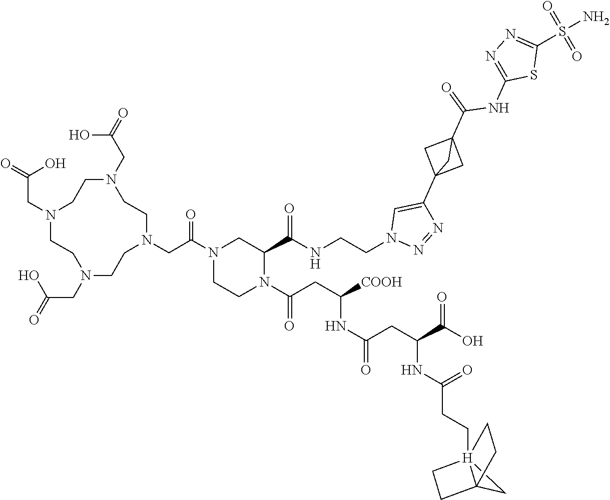 |
| 109.10 | 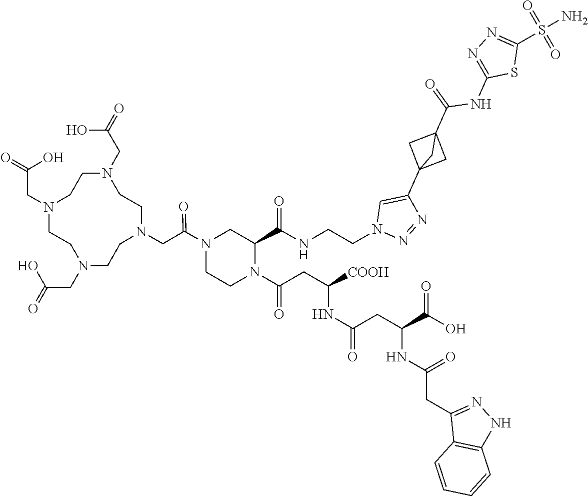 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)C[C@H](NC(=O)C[C@H](NC(=O)Cc5n[nH]c6ccccc56)C(=O)O)C(=O)O)nn4)(C2)C3)s1] |
| 109.11 | 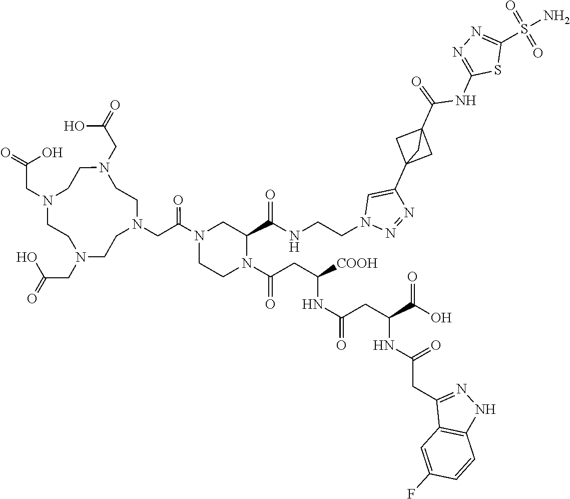 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)C[C@H](NC(=O)C[C@H](NC(=O)Cc5n[nH]c6ccc(F)cc56)C(=O)O)C(=O)O)nn4)(C2)C3)s1] |
| 109.12 | 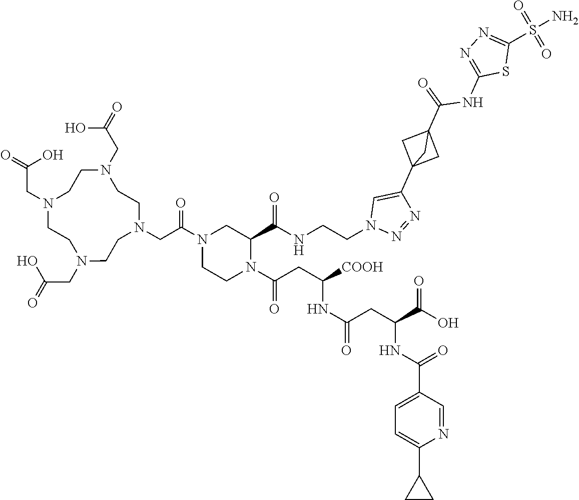 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)C[C@H](NC(=O)C[C@H](NC(=O)c5ccc(C6CC6)nc5)C(=O)O)C(=O)O)nn4)(C2)C3)s1] |
| 109.13 | 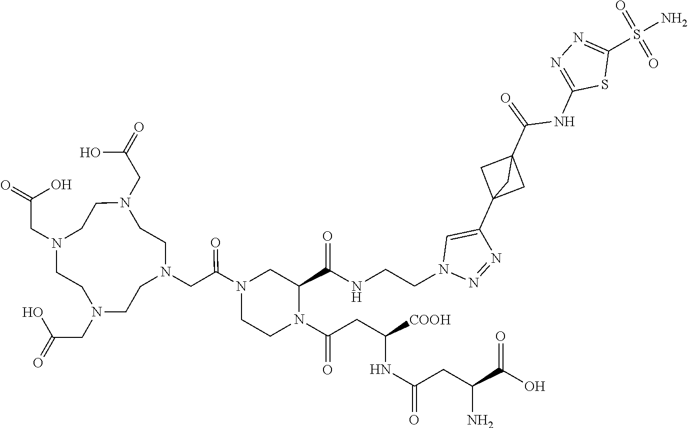 [SMILES: N[C@@H](CC(=O)N[C@@H](CC(=O)N1CCN(C(=O)CN2CCN(CC(=O)O)CCN(CC(=O)O)CCN(CC(=O)O)CC2)C[C@H]1C(=O)NCCn1cc(C23CC(C(=O)Nc4nnc(S(N)(=O)=O)s4)(C2)C3)nn1)C(=O)O)C(=O)O] |
| 109.14 | 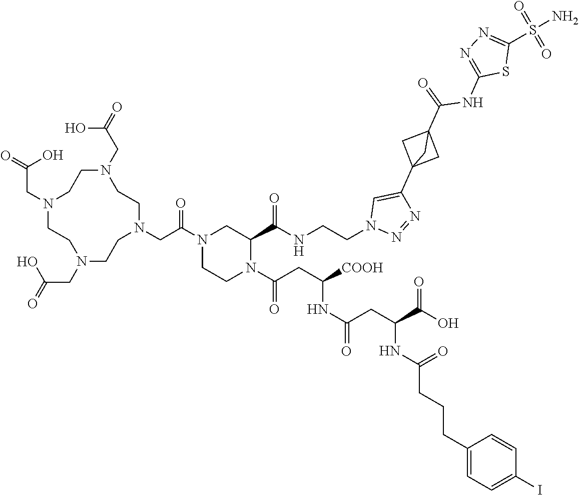 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)C[C@H](NC(=O)C[C@H](NC(=O)CCCc5ccc(I)cc5)C(=O)O)C(=O)O)nn4)(C2)C3)s1] |
| 109.15 | 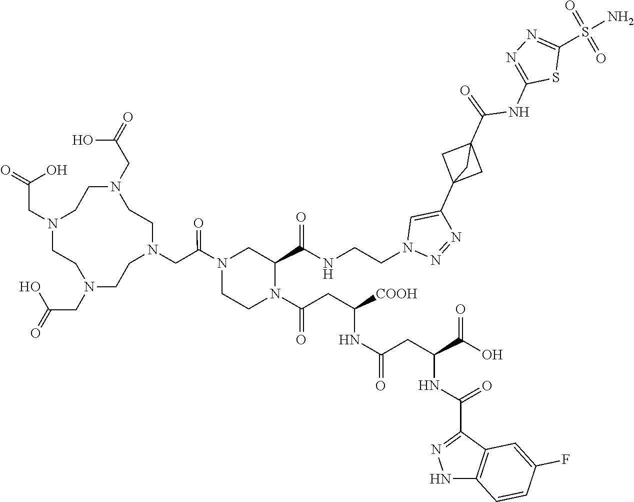 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)C[C@H](NC(=O)C[C@H](NC(=O)c5n[nH]c6ccc(F)cc56)C(=O)O)C(=O)O)nn4)(C2)C3)s1] |
| 109.16 | 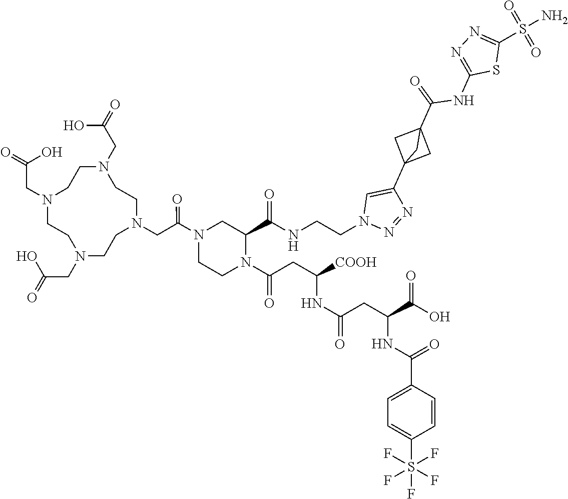 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)C[C@H](NC(=O)C[C@H](NC(=O)c5ccc(S(F)(F)(F)(F)F)cc5)C(=O)O)C(=O)O)nn4)(C2)C3)s1] |
| 109.17 | 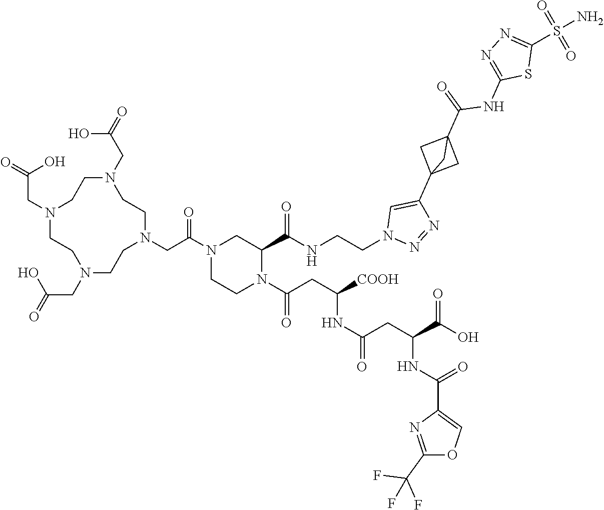 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)C[C@H](NC(=O)C[C@H](NC(=O)c5coc(C(F)(F)F)n5)C(=O)O)C(=O)O)nn4)(C2)C3)s1] |
| 109.18 | 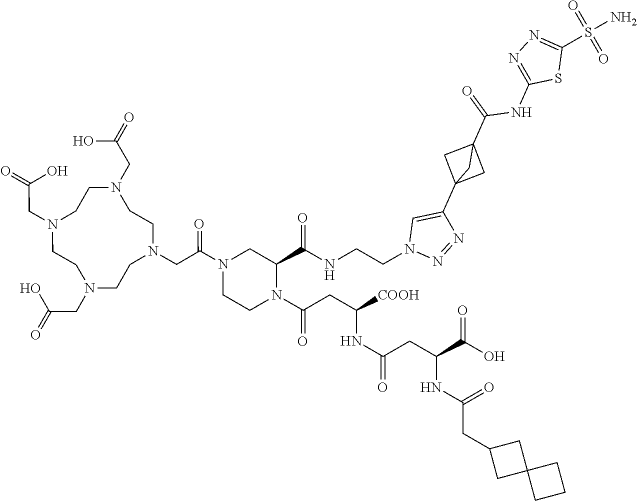 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)C[C@H](NC(=O)C[C@H](NC(=O)CC5CC6(CCC6)C5)C(=O)O)C(=O)O)nn4)(C2)C3)s1] |
| 109.19 | 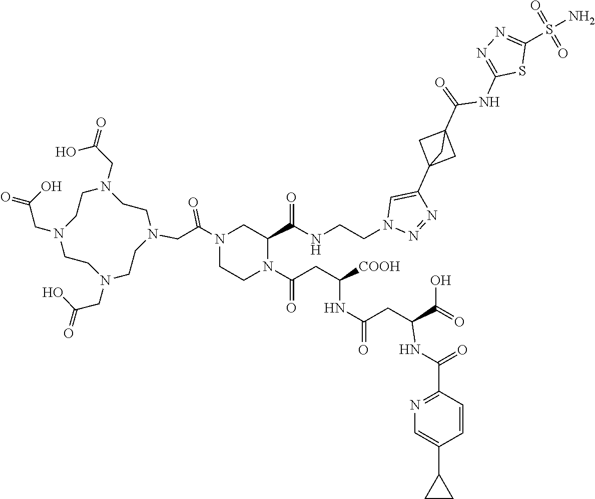 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)C[C@H](NC(=O)C[C@H](NC(=O)c5ccc(C6CC6)cn5)C(=O)O)C(=O)O)nn4)(C2)C3)s1] |
| 109.20 | 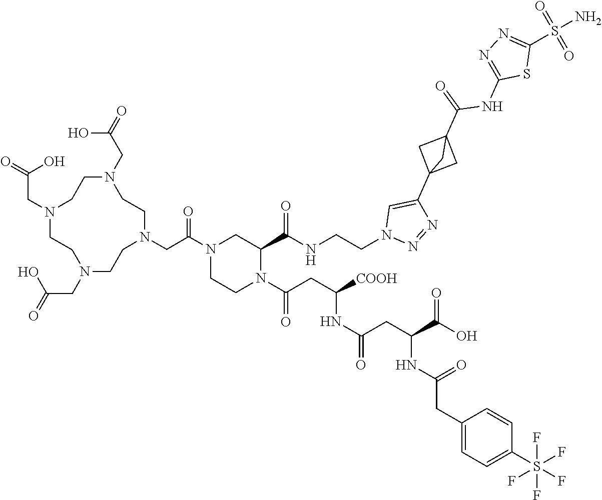 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)C[C@H](NC(=O)C[C@H](NC(=O)Cc5ccc(S(F)(F)(F)(F)F)cc5)C(=O)O)C(=O)O)nn4)(C2)C3)s1] |
| 109.21 | 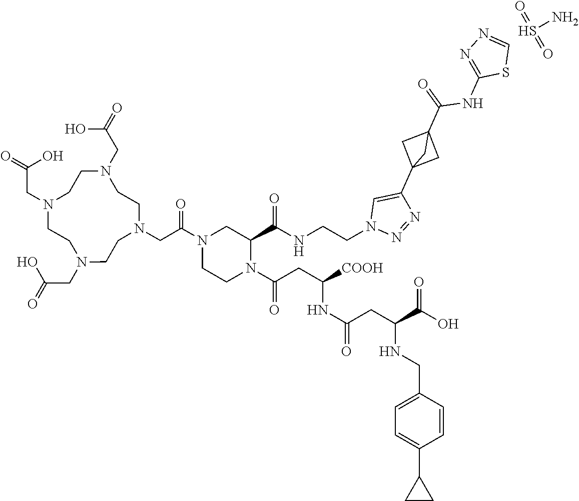 |
| 109.22 | 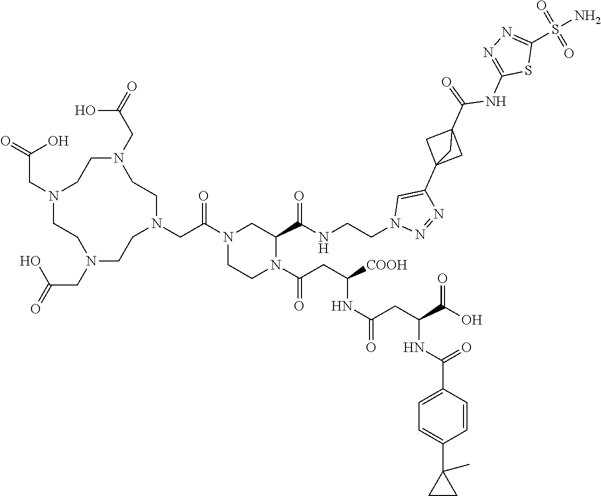 |
| 109.23 | 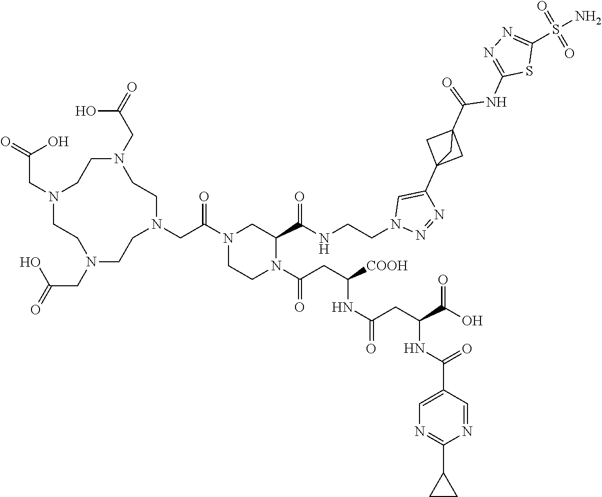 |
| 109.24 | 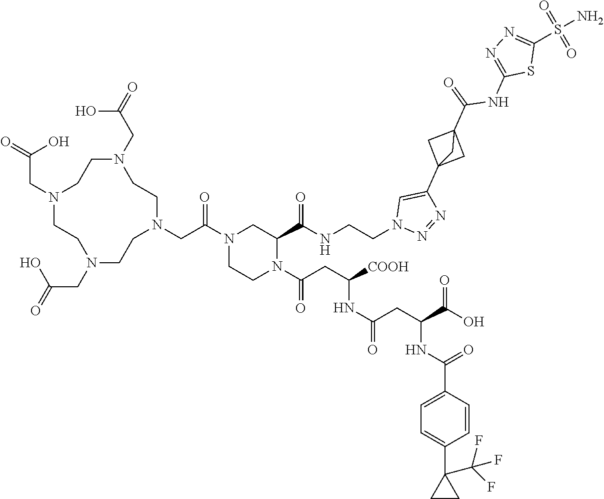 |
| 109.25 | 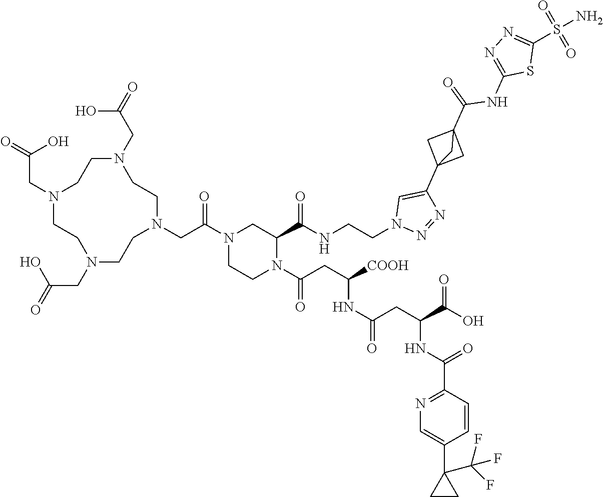 |
| 109.26 | 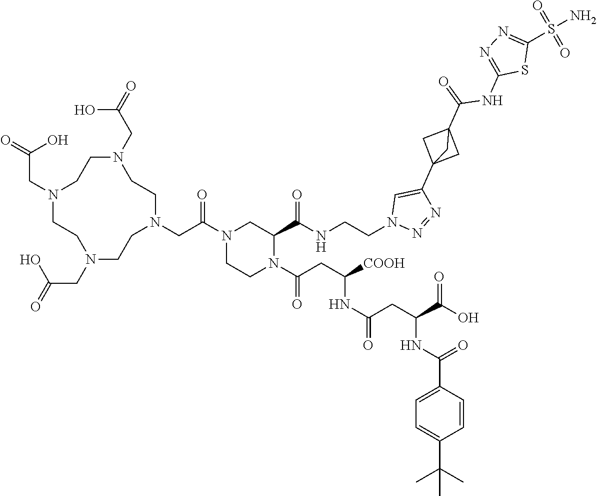 |
| 109.27 | 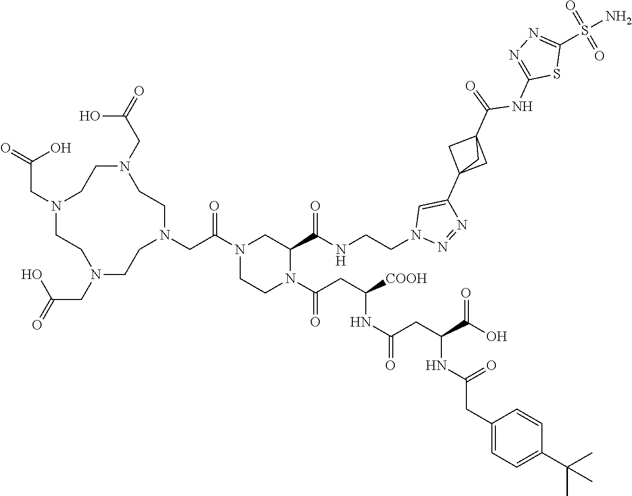 [SMILES: CC(C)(C)c1ccc(CC(=O)N[C@@H](CC(=O)N[C@@H](CC(=O)N2CCN(C(=O)CN3CCN(CC(=O)O)CCN(CC(=O)O)CCN(CC(=O)O)CC3)C[C@H]2C(=O)NCCn2cc(C34CC(C(=O)Nc5nnc(S(N)(=O)=O)s5)(C3)C4)nn2)C(=O)O)C(=O)O)cc1] |
| 109.28 | 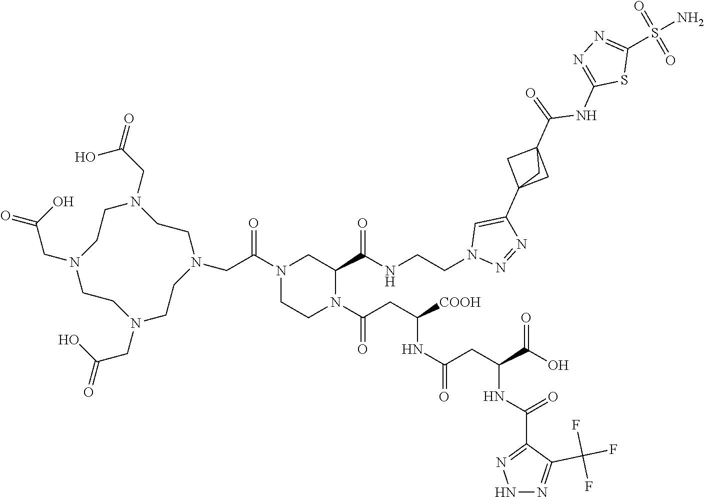 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)C[C@H](NC(=O)C[C@H](NC(=O)c5n[nH]nc5C(F)(F)F)C(=O)O)C(=O)O)nn4)(C2)C3)s1] |
| 109.29 | 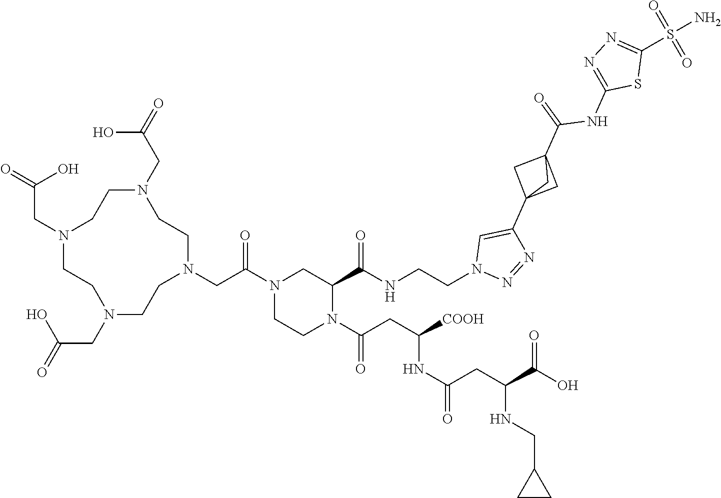 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)C[C@H](NC(=O)C[C@H](NCC5CC5)C(=O)O)C(=O)O)nn4)(C2)C3)s1] |
| 109.30 | 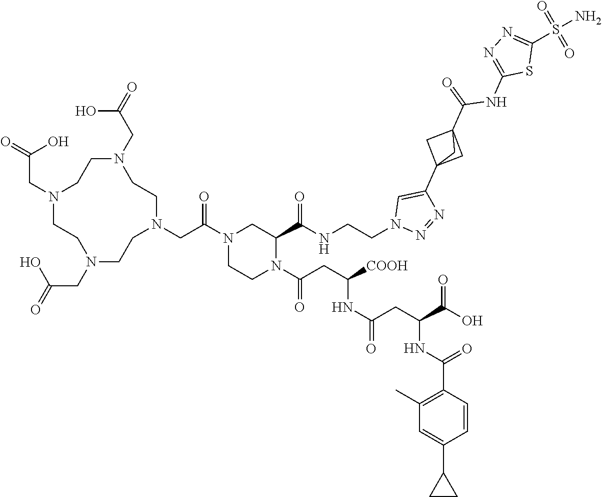 |
| 109.31 | 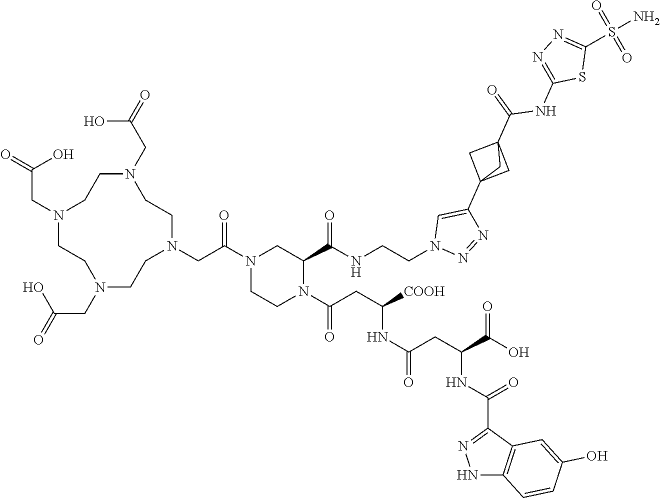 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)C[C@H](NC(=O)C[C@H](NC(=O)c5n[nH]c6ccc(O)cc56)C(=O)O)C(=O)O)nn4)(C2)C3)s1] |
| 109.32 | 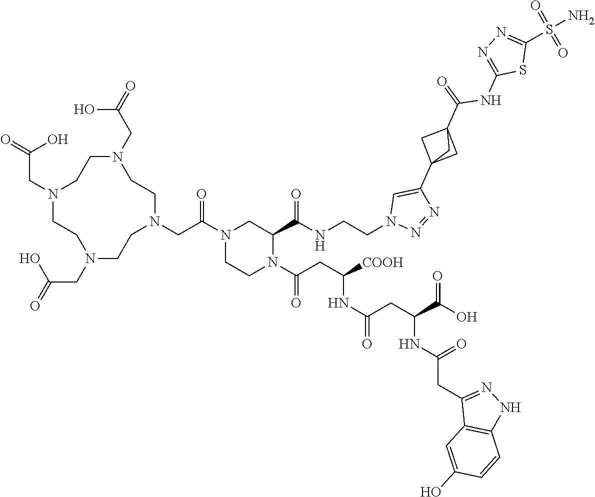 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)C[C@H](NC(=O)C[C@H](NC(=O)Cc5n[nH]c6ccc(O)cc56)C(=O)O)C(=O)O)nn4)(C2)C3)s1] |
| 109.33 | 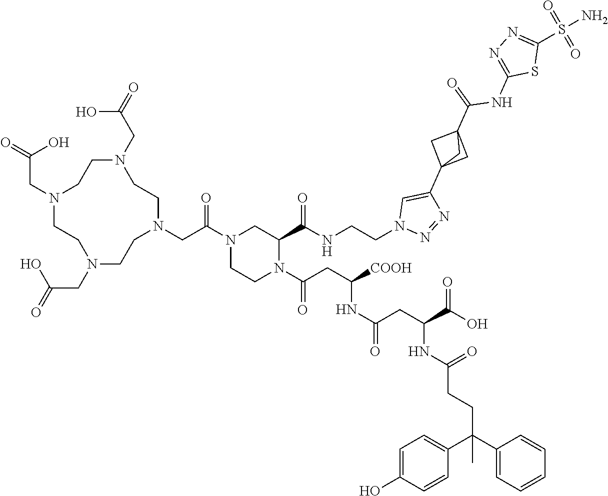 [SMILES: CC(CCC(=O)N[C@@H](CC(=O)N[C@@H](CC(=O)N1CCN(C(=O)CN2CCN(CC(=O)O)CCN(CC(=O)O)CCN(CC(=O)O)CC2)C[C@H]1C(=O)NCCn1cc(C23CC(C(=O)Nc4nnc(S(N)(=O)=O)s4)(C2)C3)nn1)C(=O)O)C(=O)O)(c1ccccc1)c1ccc(O)cc1] |
| 109.34 | 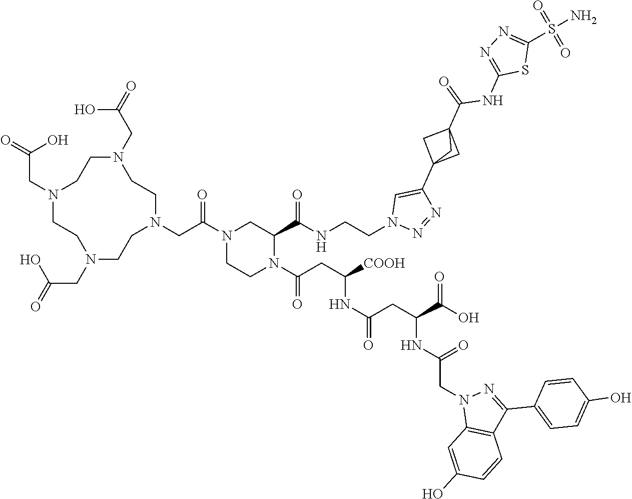 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)C[C@H](NC(=O)C[C@H](NC(=O)Cn5nc(-c6ccc(O)cc6)c6ccc(O)cc65)C(=O)O)C(=O)O)nn4)(C2)C3)s1] |
| 109.35 | 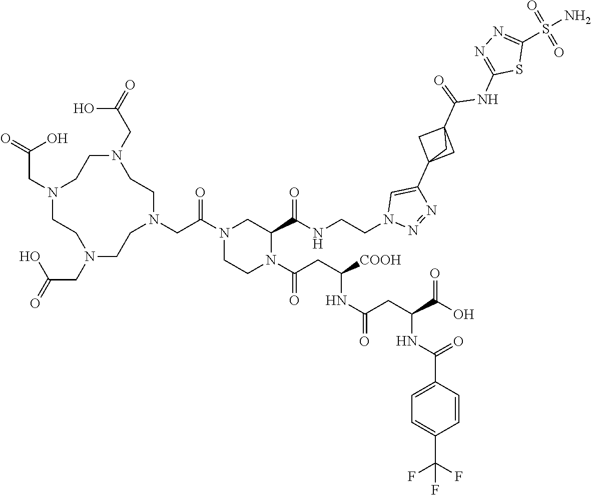 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)C[C@H](NC(=O)C[C@H](NC(=O)c5ccc(C(F)(F)F)cc5)C(=O)O)C(=O)O)nn4)(C2)C3)s1] |
| 109.36 | 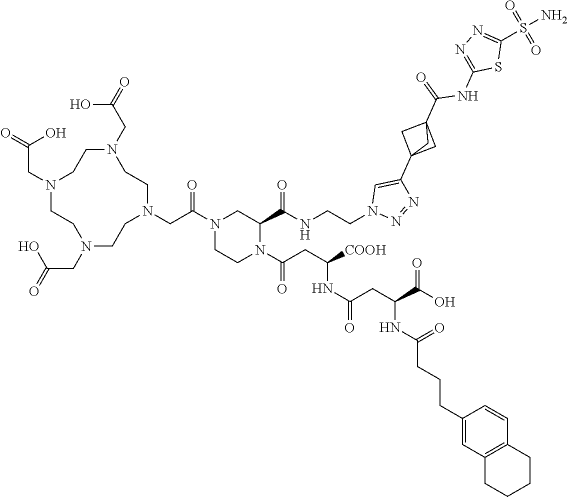 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)C[C@H](NC(=O)C[C@H](NC(=O)CCCc5ccc6c(c5)CCCC6)C(=O)O)C(=O)O)nn4)(C2)C3)s1] |
| 109.37 | 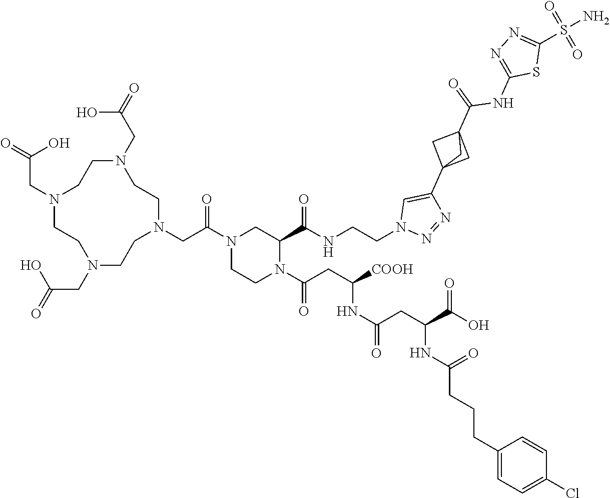 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)C[C@H](NC(=O)C[C@H](NC(=O)CCCc5ccc(Cl)cc5)C(=O)O)C(=O)O)nn4)(C2)C3)s1] |
| 109.38 | 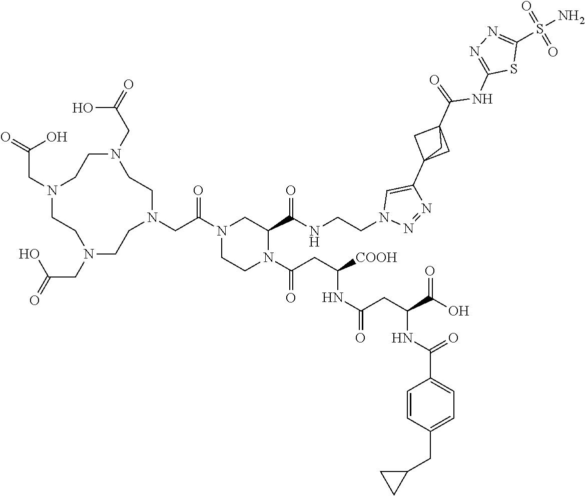 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)C[C@H](NC(=O)C[C@H](NC(=O)c5ccc(CC6CC6)cc5)C(=O)O)C(=O)O)nn4)(C2)C3)s1] |
| 109.39 | 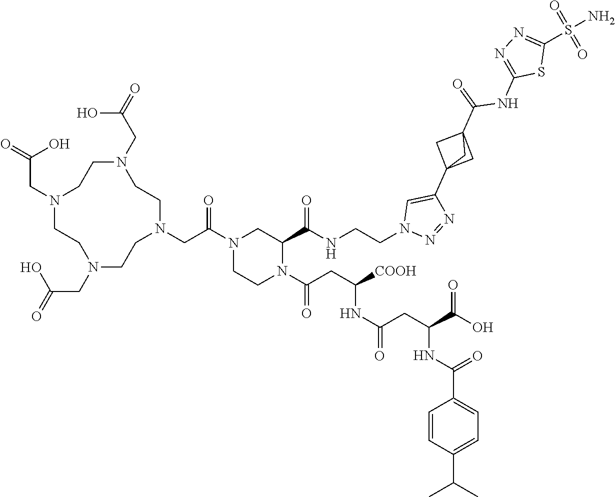 |
| 109.40 | 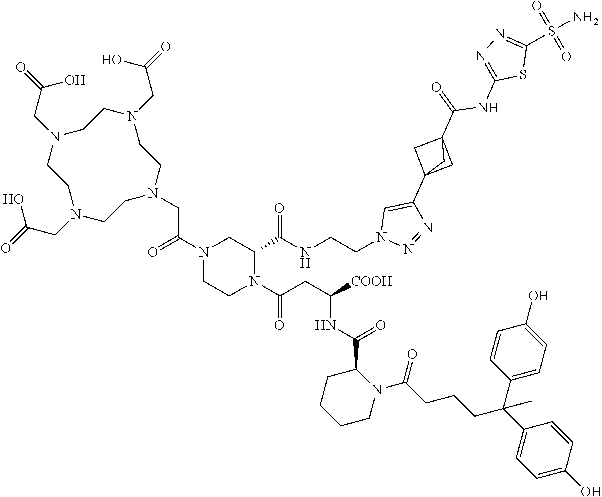 [SMILES: CC(CCCC(=O)N1CCCC[C@H]1C(=O)N[C@@H](CC(=O)N1CCN(C(=O)CN2CCN(CC(=O)O)CCN(CC(=O)O)CCN(CC(=O)O)CC2)C[C@@H]1C(=O)NCCn1cc(C23CC(C(=O)Nc4nnc(S(N)(=O)=O)s4)(C2)C3)nn1)C(=O)O)(c1ccc(O)cc1)c1ccc(O)cc1] |
| 109.41 | 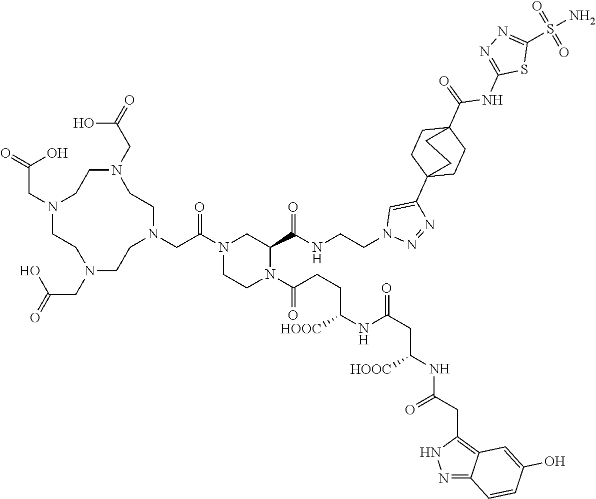 |
| 109.42 | 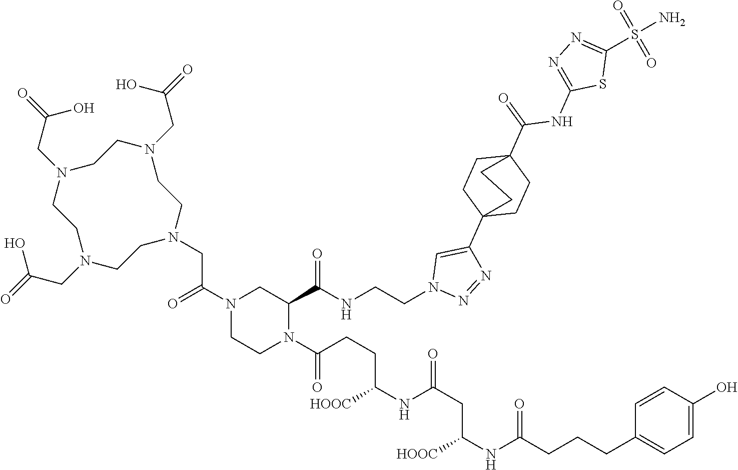 |
| 109.43 | 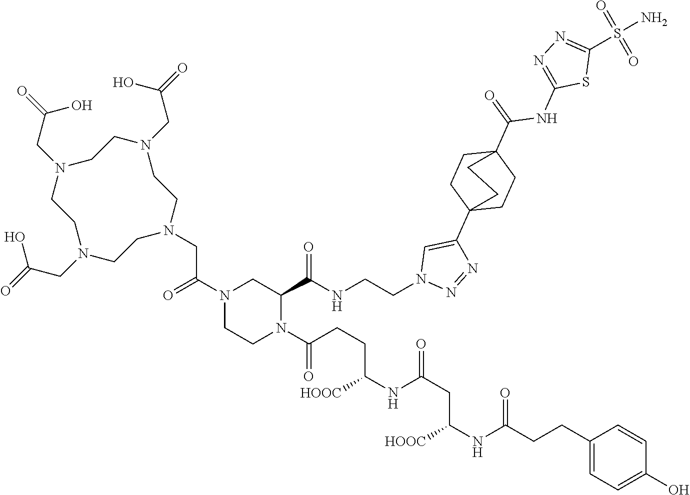 |
| 109.44 | 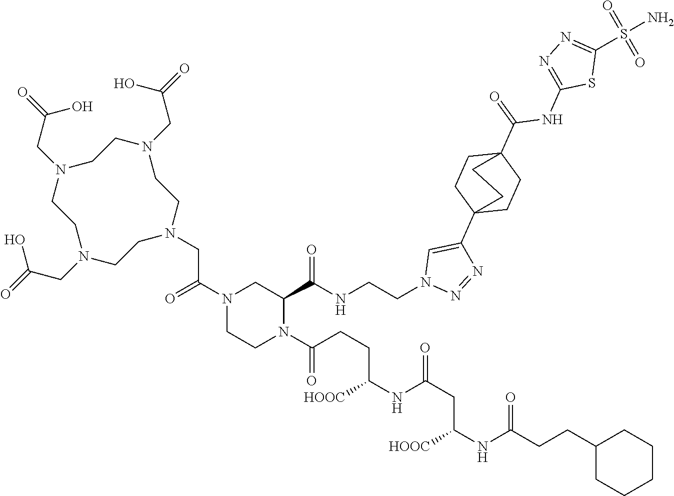 |
| 109.45 | 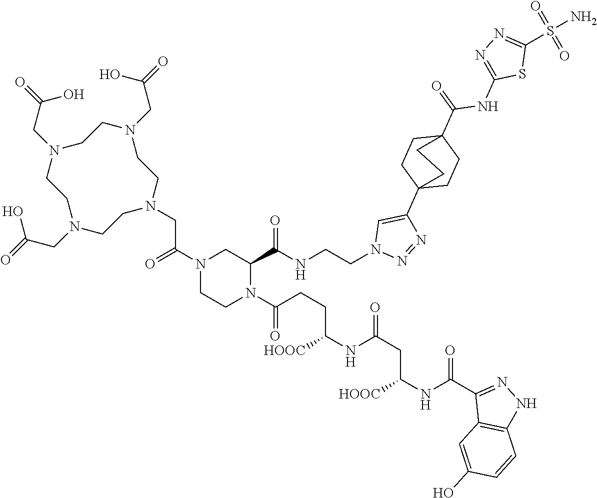 |
| 109.46 | 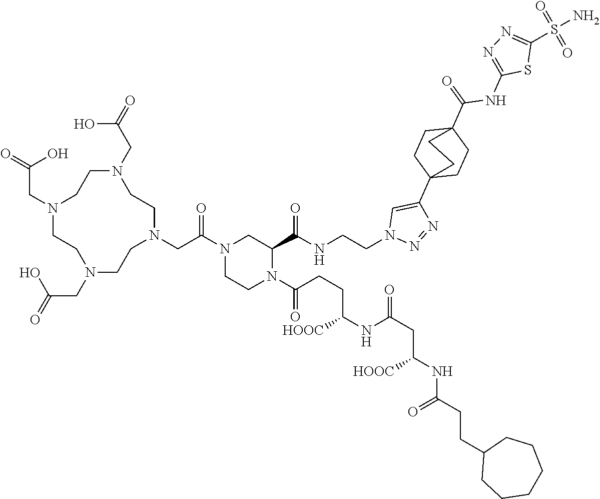 |
| 109.47 | 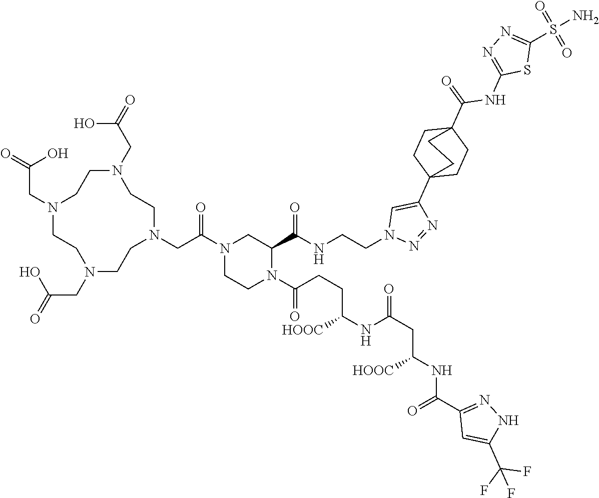 |
| 109.48 | 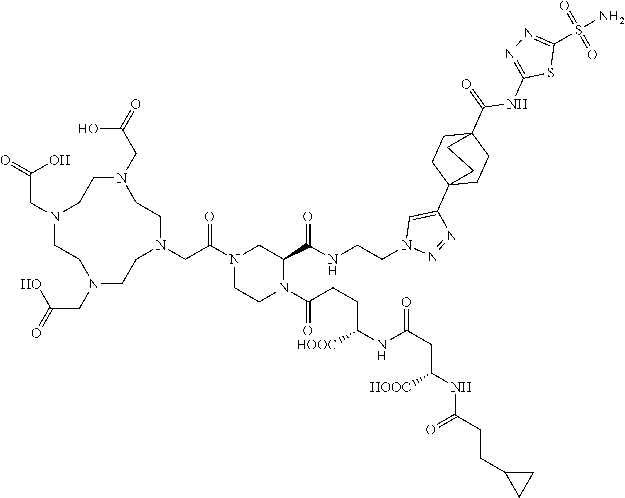 |
| 109.49 | 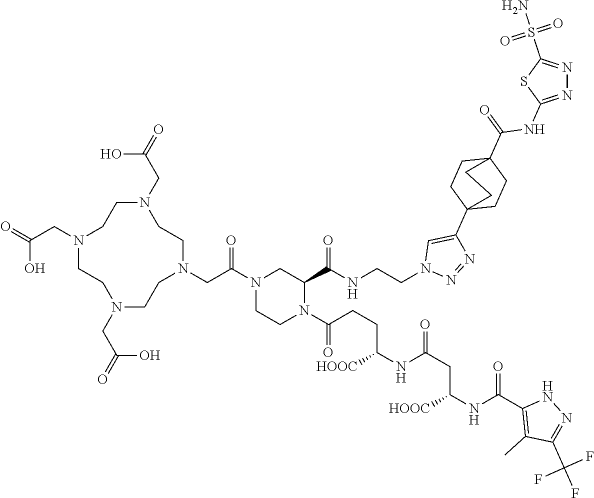 |
| 109.50 | 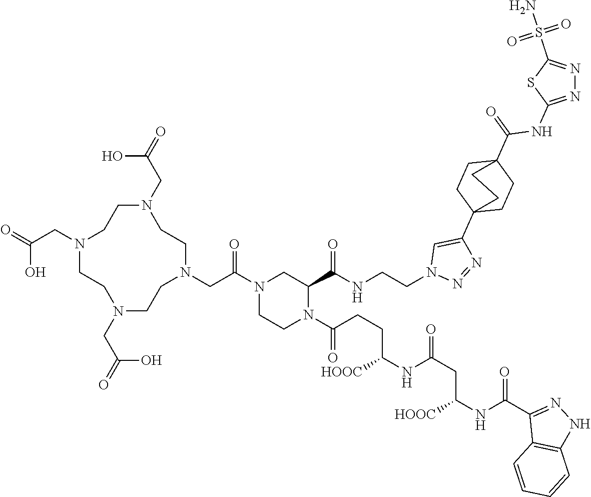 |
| 109.51 | 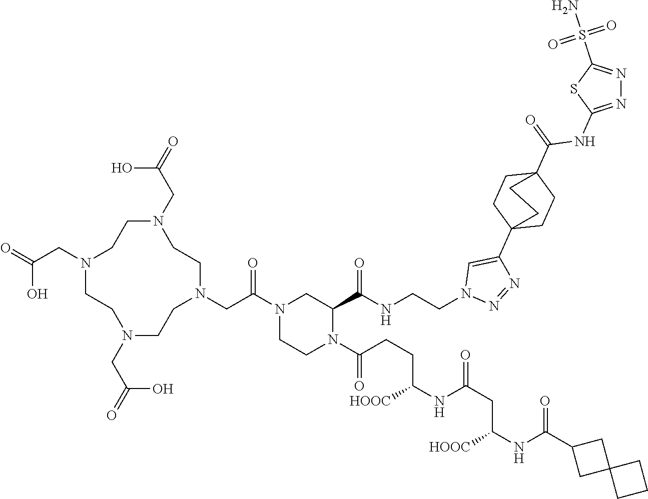 |
| 109.52 | 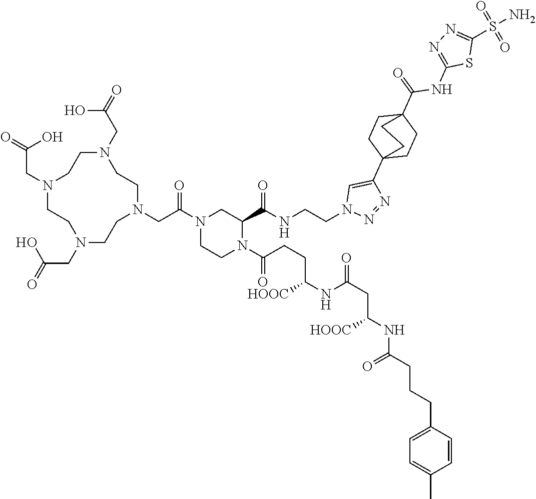 |
| 109.53 | 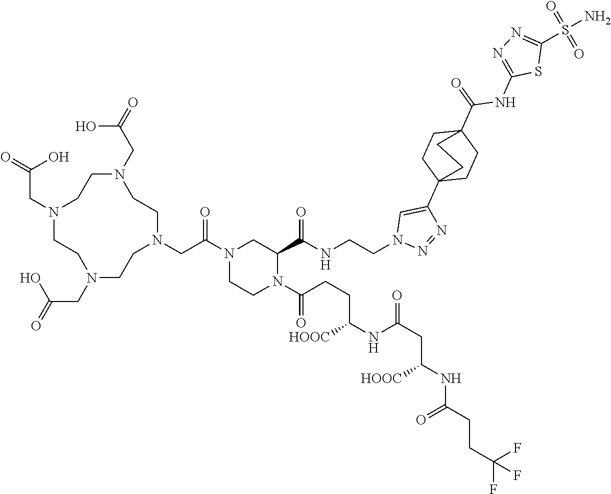 |
| 109.54 | 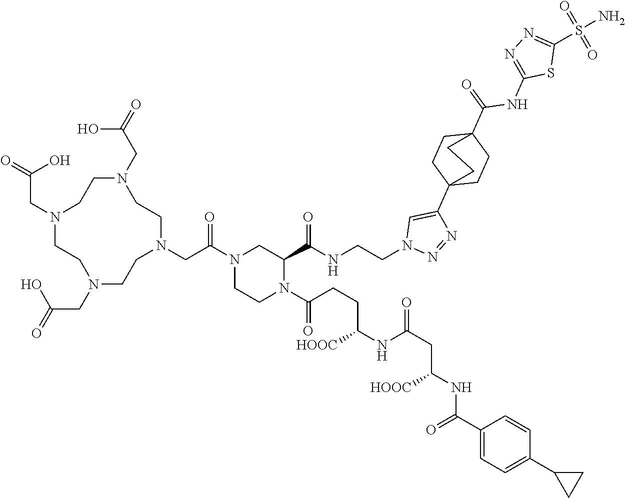 |
| 109.55 | 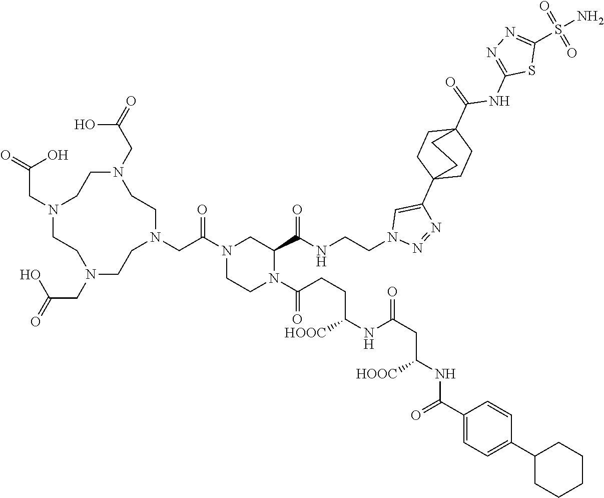 |
| 109.56 | 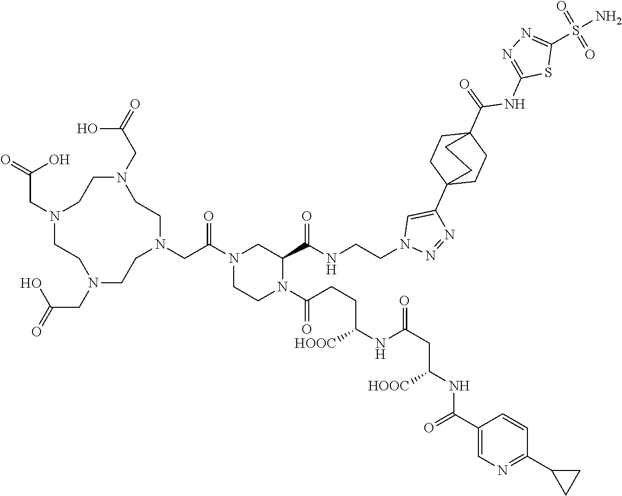 |
| 109.57 | 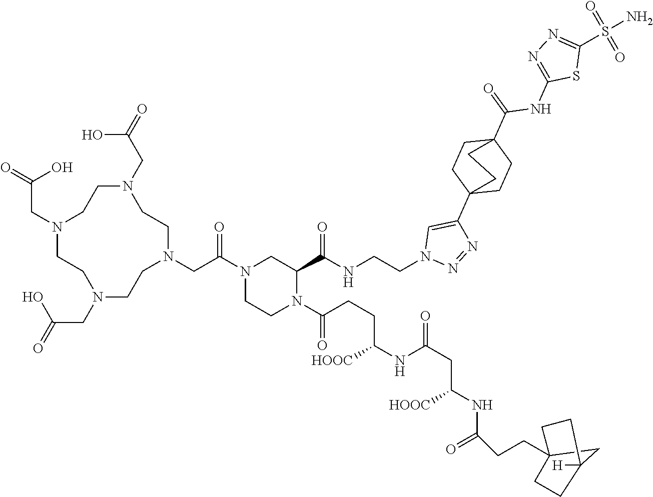 |
| 109.58 | 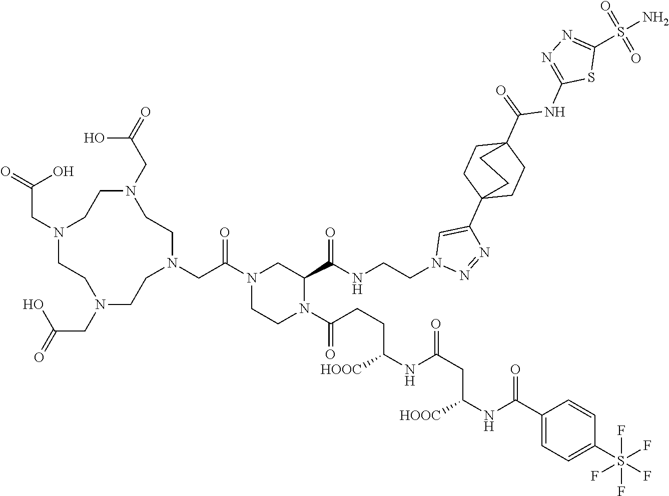 |
| 109.59 | 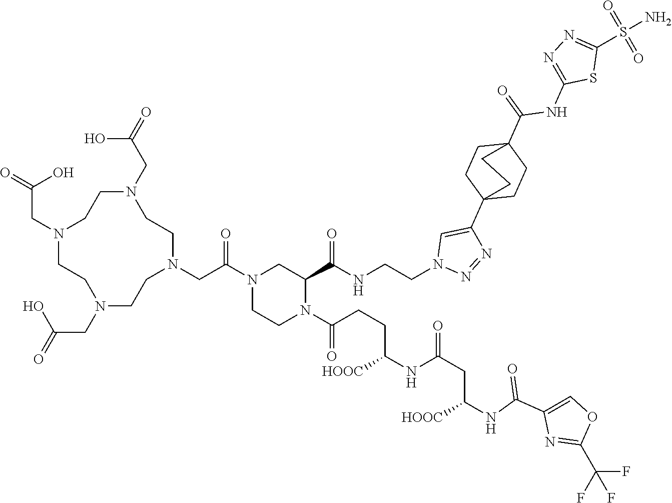 |
| 109.60 | 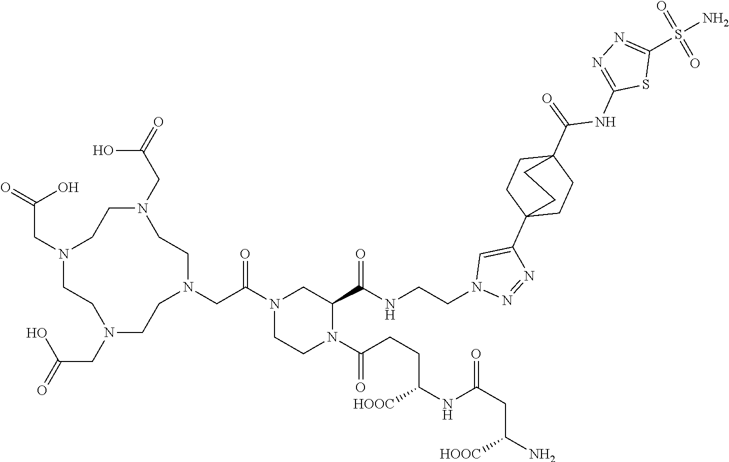 |
| 109.61 | 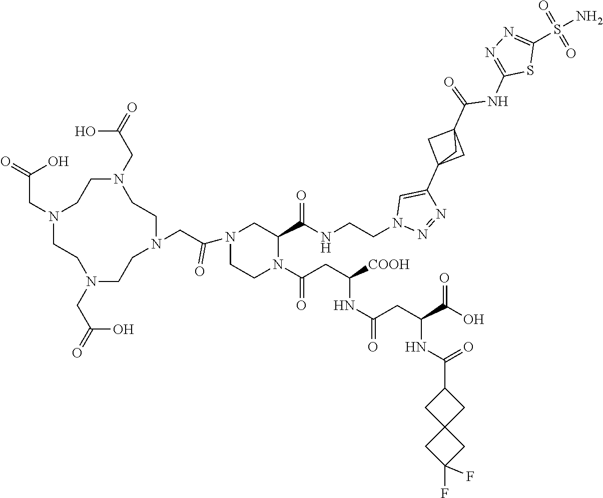 |
| 109.62 | 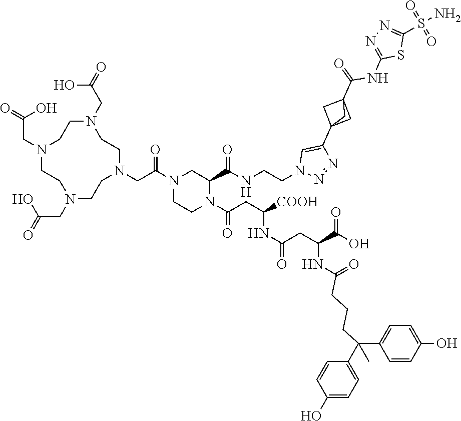 [SMILES: CC(CCCC(=O)N[C@@H](CC(=O)N[C@@H](CC(=O)N1CCN(C(=O)CN2CCN(CC(=O)O)CCN(CC(=O)O)CCN(CC(=O)O)CC2)C[C@H]1C(=O)NCCn1cc(C23CC(C(=O)Nc4nnc(S(N)(=O)=O)s4)(C2)C3)nn1)C(=O)O)C(=O)O)(c1ccc(O)cc1)c1ccc(O)cc1] |
| 109.63 | 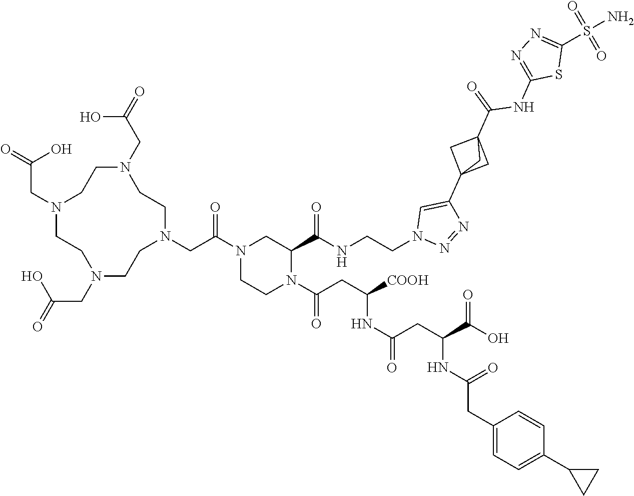 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)C[C@H](NC(=O)C[C@H](NC(=O)Cc5ccc(C6CC6)cc5)C(=O)O)C(=O)O)nn4)(C2)C3)s1] |
| 110 | 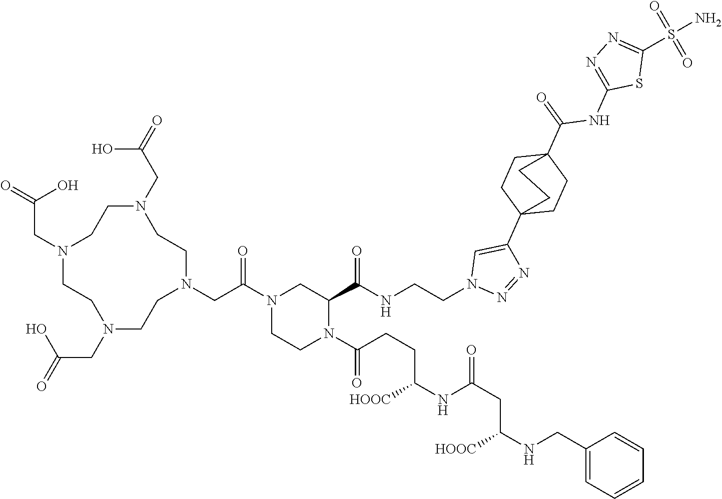 |
| 111 | 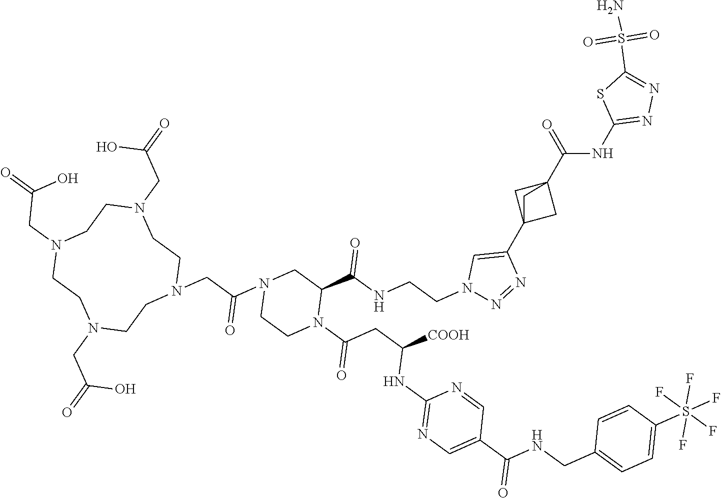 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)C[C@H](Nc5ncc(C(=O)NCc6ccc(S(F)(F)(F)(F)F)cc6)cn5)C(=O)O)nn4)(C2)C3)s1] |
| 111.1 | 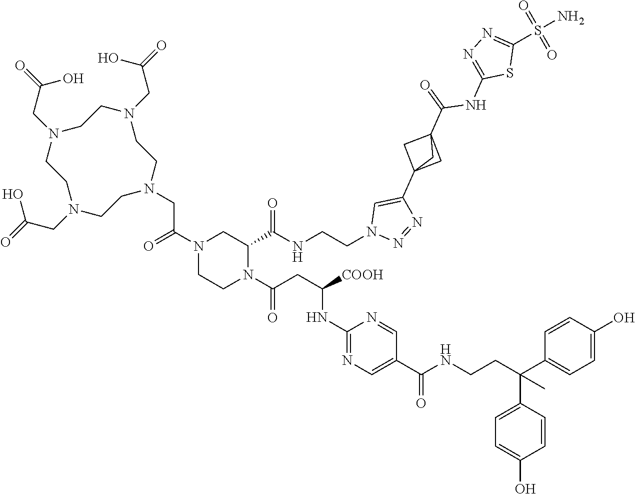 |
| 111.2 | 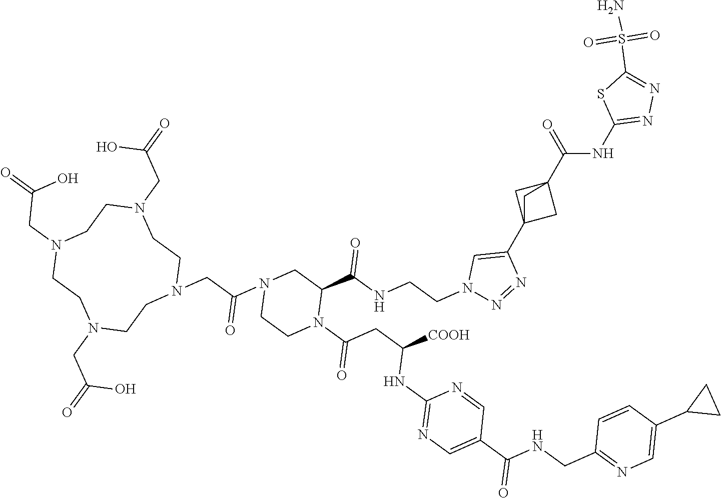 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)C[C@H](Nc5ncc(C(=O)NCc6ccc(C7CC7)cn6)cn5)C(=O)O)nn4)(C2)C3)s1] |
| 111.3 | 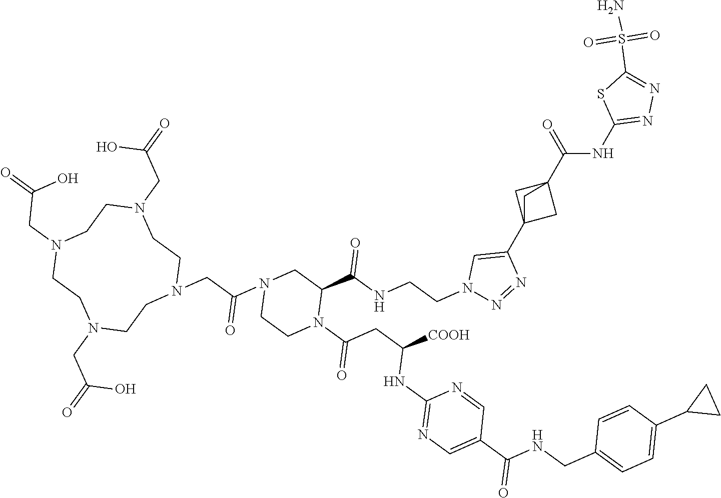 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)C[C@H](Nc5ncc(C(=O)NCc6ccc(C7CC7)cc6)cn5)C(=O)O)nn4)(C2)C3)s1] |
| 111.4 | 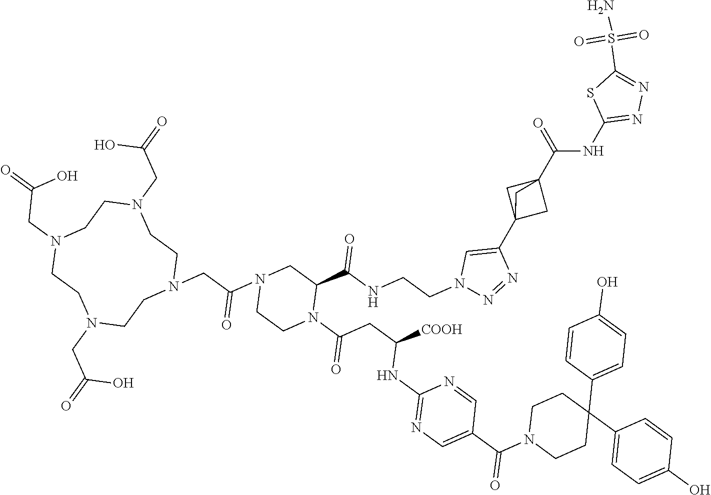 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)C[C@H](Nc5ncc(C(=O)N6CCC(c7ccc(O)cc7)(c7ccc(O)cc7)CC6)cn5)C(=O)O)nn4)(C2)C3)s1] |
| 111.5 | 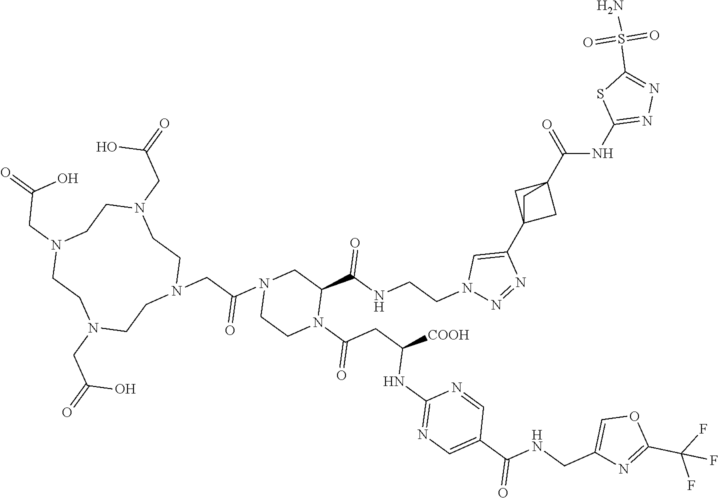 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)C[C@H](Nc5ncc(C(=O)NCc6coc(C(F)(F)F)n6)cn5)C(=O)O)nn4)(C2)C3)s1] |
| 111.6 | 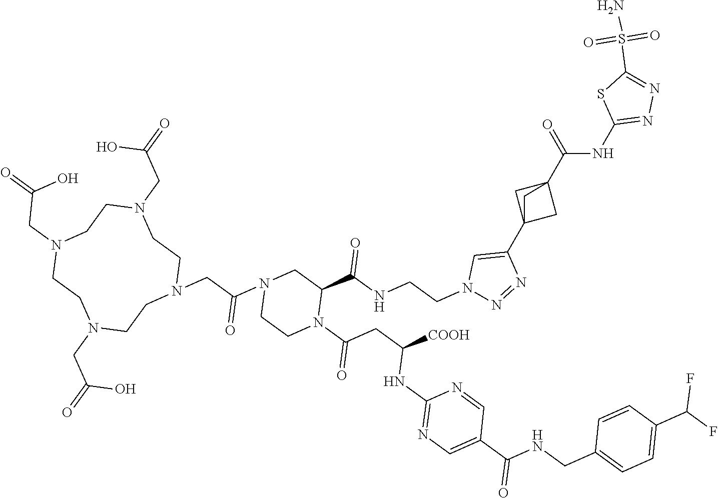 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)C[C@H](Nc5ncc(C(=O)NCc6ccc(C(F)F)cc6)cn5)C(=O)O)nn4)(C2)C3)s1] |
| 111.7 | 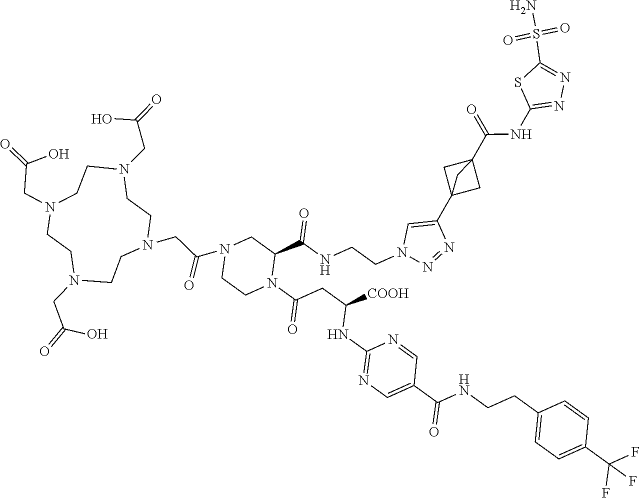 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)C[C@H](Nc5ncc(C(=O)NCCc6ccc(C(F)(F)F)cc6)cn5)C(=O)O)nn4)(C2)C3)s1] |
| 111.8 | 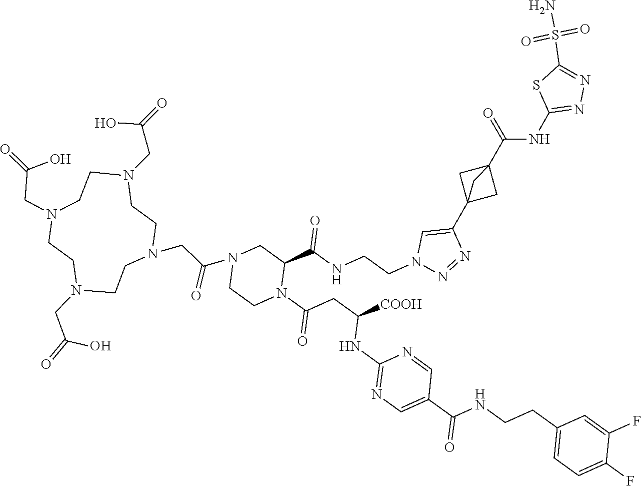 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)C[C@H](Nc5ncc(C(=O)NCCc6ccc(F)c(F)c6)cn5)C(=O)O)nn4)(C2)C3)s1] |
| 111.9 | 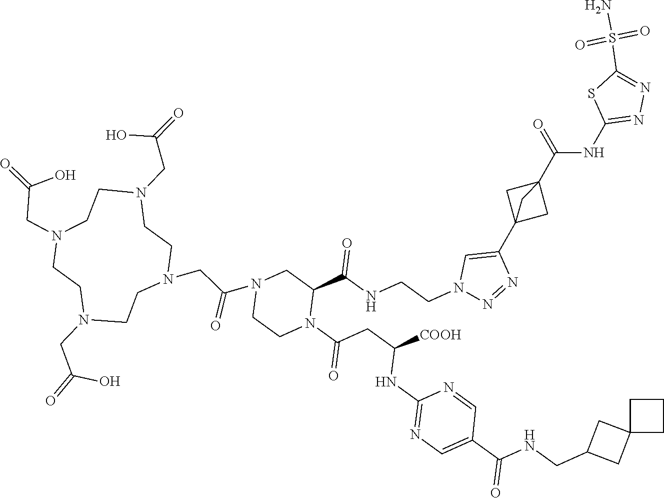 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)C[C@H](Nc5ncc(C(=O)NCC6CC7(CCC7)C6)cn5)C(=O)O)nn4)(C2)C3)s1] |
| 111.10 | 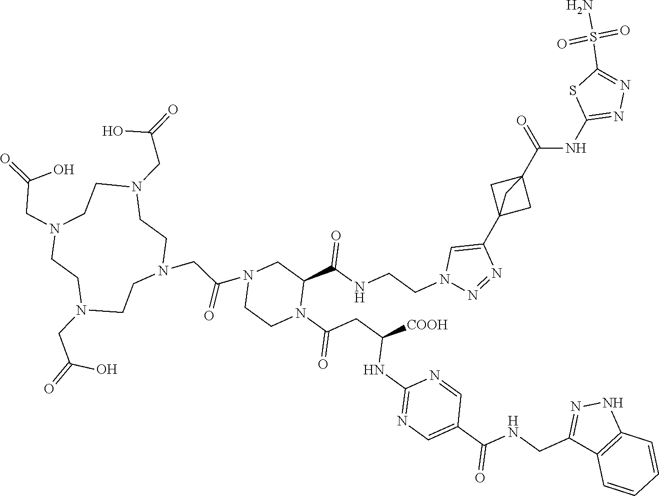 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)C[C@H](Nc5ncc(C(=O)NCc6n[nH]c7ccccc67)cn5)C(=O)O)nn4)(C2)C3)s1] |
| 111.11 | 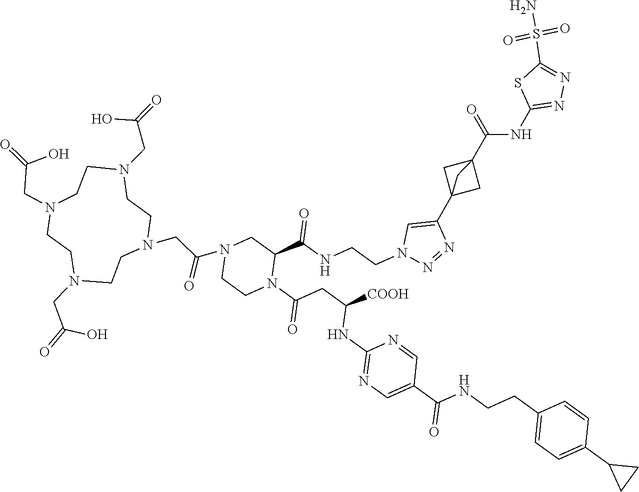 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)C[C@H](Nc5ncc(C(=O)NCCc6ccc(C7CC7)cc6)cn5)C(=O)O)nn4)(C2)C3)s1] |
| 111.12 | 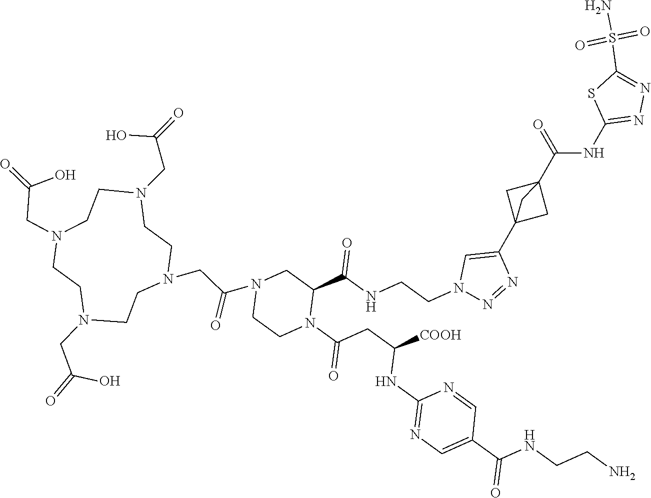 [SMILES: NCCNC(=O)c1cnc(N[C@@H](CC(=O)N2CCN(C(=O)CN3CCN(CC(=O)O)CCN(CC(=O)O)CCN(CC(=O)O)CC3)C[C@H]2C(=O)NCCn2cc(C34CC(C(=O)Nc5nnc(S(N)(=O)=O)s5)(C3)C4)nn2)C(=O)O)nc1] |
| 112 | 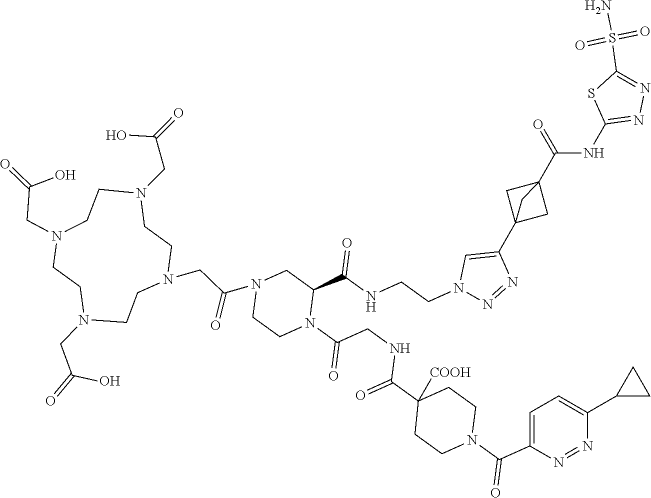 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)CNC(=O)C5(C(=O)O)CCN(C(=O)c6ccc(C7CC7)nn6)CC5)nn4)(C2)C3)s1] |
| 112.1 | 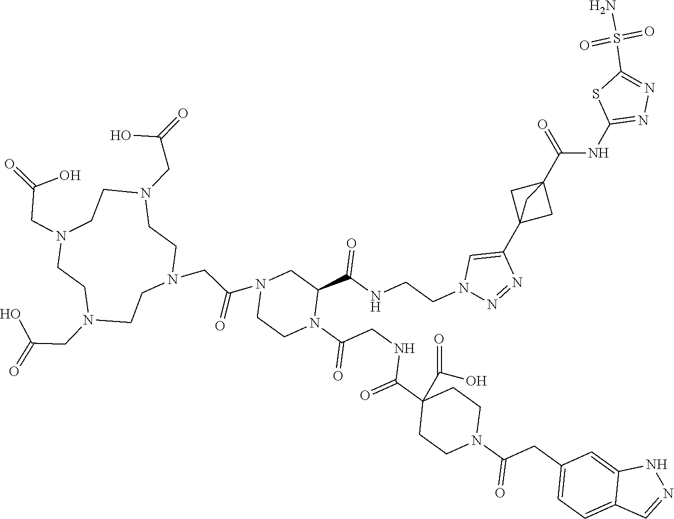 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)CNC(=O)C5(C(=O)O)CCN(C(=O)Cc6ccc7cn[nH]c7c6)CC5)nn4)(C2)C3)s1] |
| 112.2 | 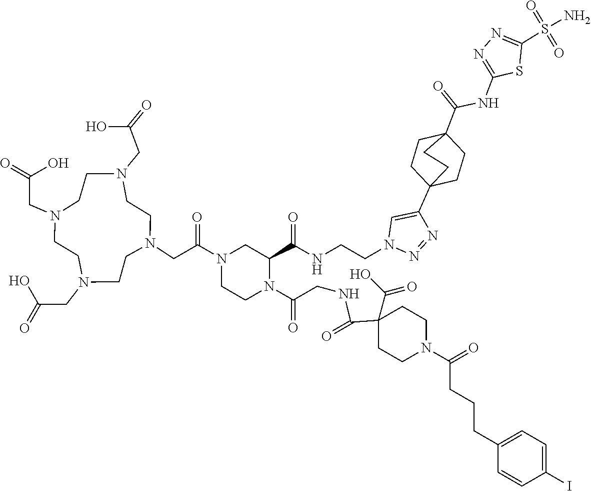 [SMILES: NS(=O)(=O)c1nnc(NC(=O)C23CCC(c4cn(CCNC(=O)[C@@H]5CN(C(=O)CN6CCN(CC(=O)O)CCN(CC(=O)O)CCN(CC(=O)O)CC6)CCN5C(=O)CNC(=O)C5(C(=O)O)CCN(C(=O)CCCc6ccc(I)cc6)CC5)nn4)(CC2)CC3)s1] |
| 112.3 | 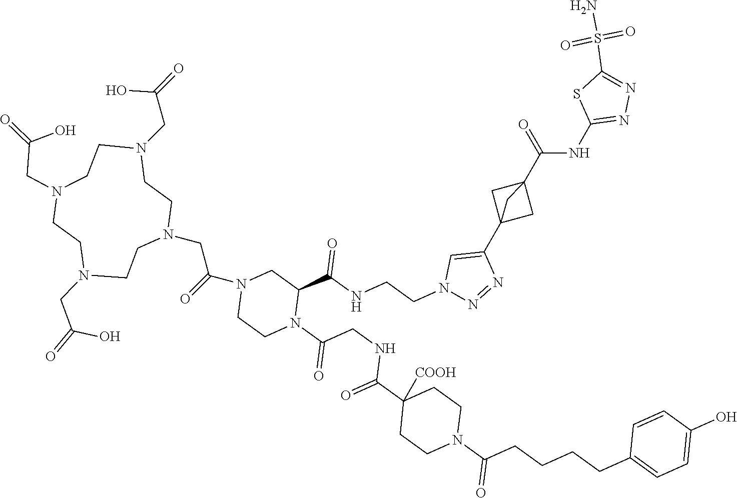 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)CNC(=O)C5(C(=O)O)CCN(C(=O)CCCCc6ccc(O)cc6)CC5)nn4)(C2)C3)s1] |
| 112.4 | 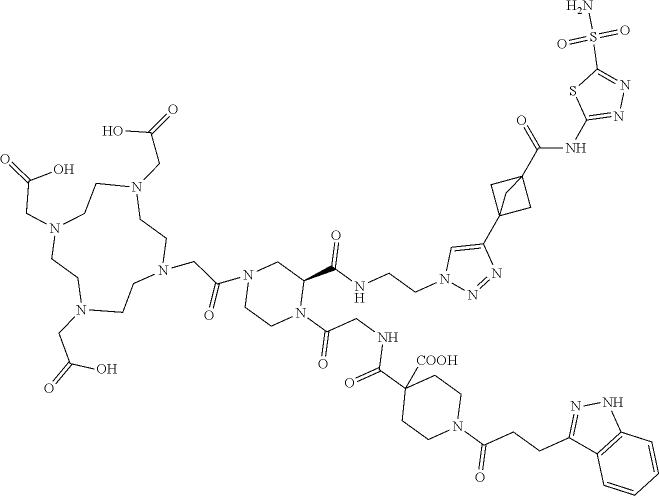 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)CNC(=O)C5(C(=O)O)CCN(C(=O)CCc6n[nH]c7ccccc67)CC5)nn4)(C2)C3)s1] |
| 112.5 | 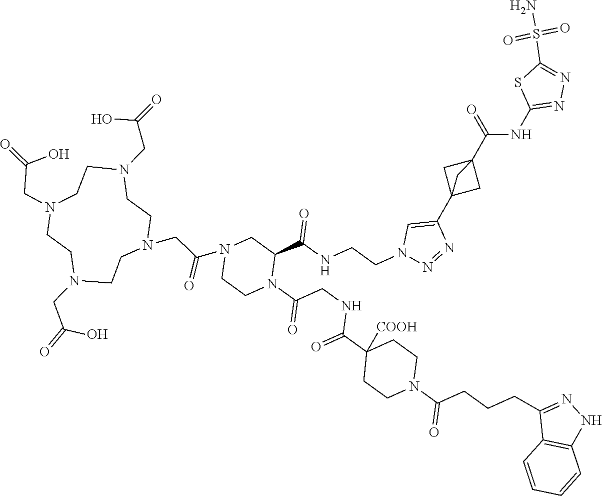 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)CNC(=O)C5(C(=O)O)CCN(C(=O)CCCc6n[nH]c7ccccc67)CC5)nn4)(C2)C3)s1] |
| 112.6 | 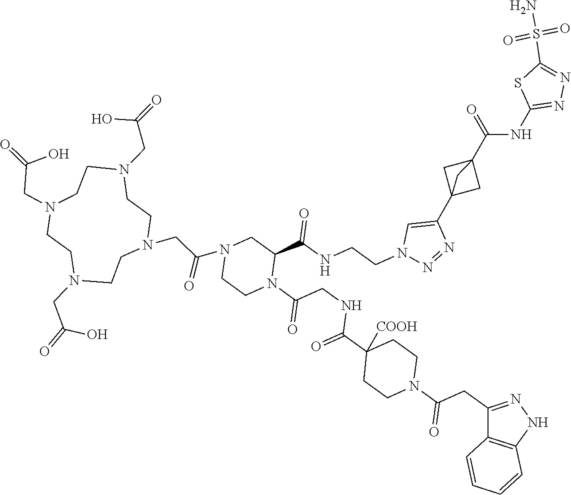 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)CNC(=O)C5(C(=O)O)CCN(C(=O)Cc6n[nH]c7ccccc67)CC5)nn4)(C2)C3)s1] |
| 112.7 | 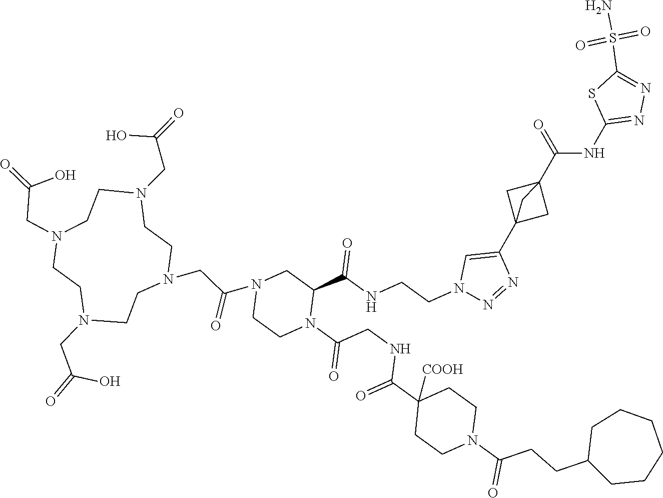 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)CNC(=O)C5(C(=O)O)CCN(C(=O)CCC6CCCCCC6)CC5)nn4)(C2)C3)s1] |
| 112.8 | 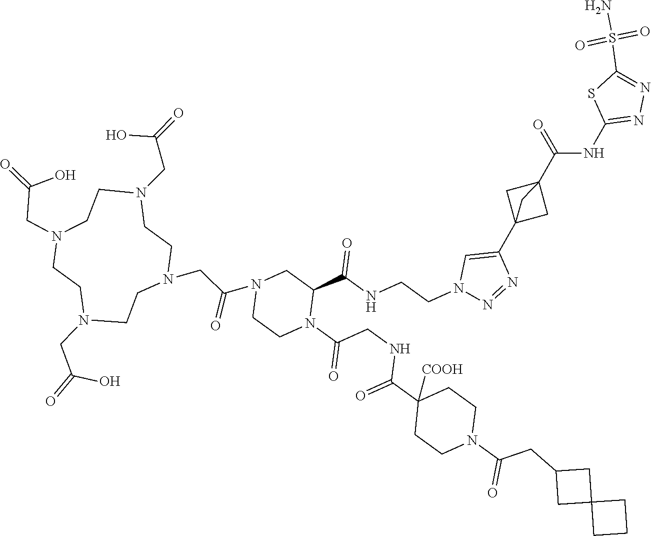 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)CNC(=O)C5(C(=O)O)CCN(C(=O)CC6CC7(CCC7)C6)CC5)nn4)(C2)C3)s1] |
| 112.9 | 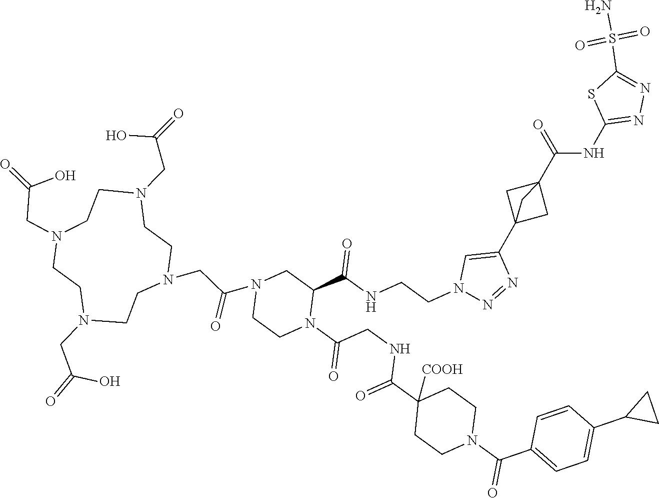 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)CNC(=O)C5(C(=O)O)CCN(C(=O)c6ccc(C7CC7)cc6)CC5)nn4)(C2)C3)s1] |
| 112.10 | 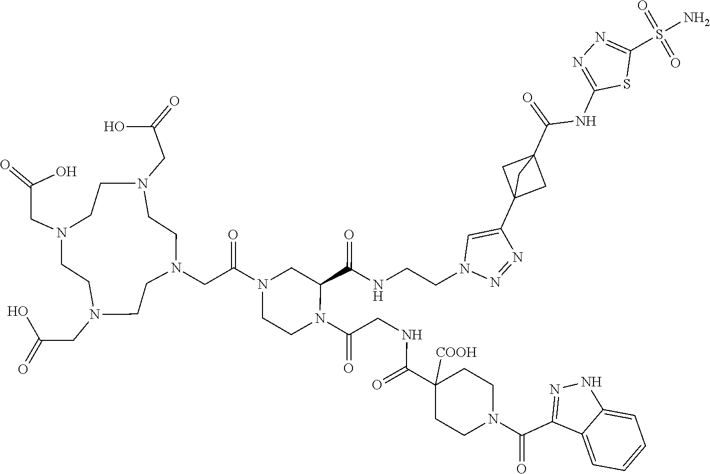 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)CNC(=O)C5(C(=O)O)CCN(C(=O)c6n[nH]c7ccccc67)CC5)nn4)(C2)C3)s1] |
| 112.11 | 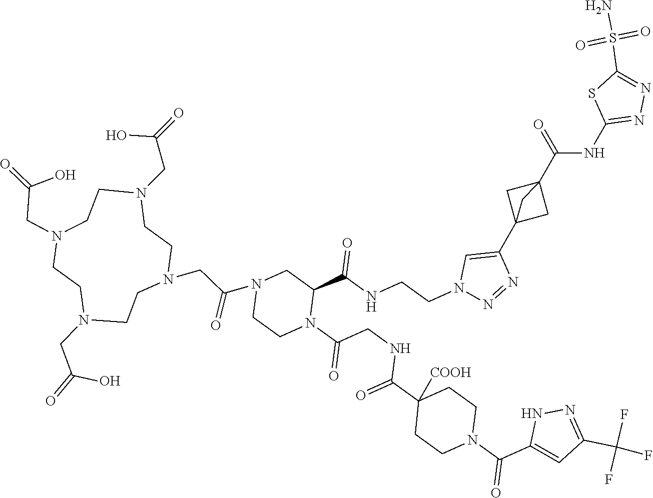 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)CNC(=O)C5(C(=O)O)CCN(C(=O)c6cc(C(F)(F)F)n[nH]6)CC5)nn4)(C2)C3)s1] |
| 112.12 | 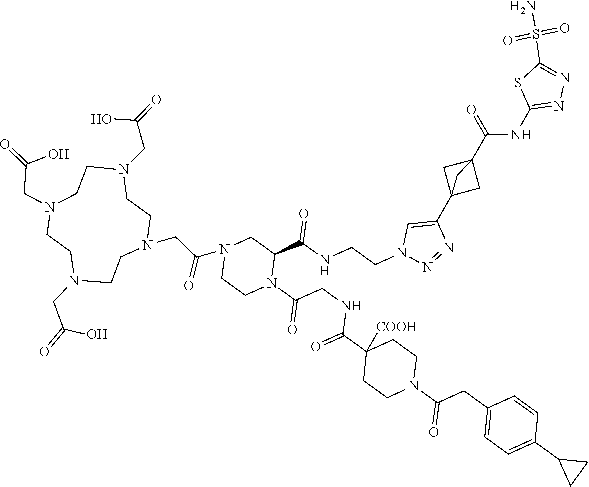 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)CNC(=O)C5(C(=O)O)CCN(C(=O)Cc6ccc(C7CC7)cc6)CC5)nn4)(C2)C3)s1] |
| 112.13 | 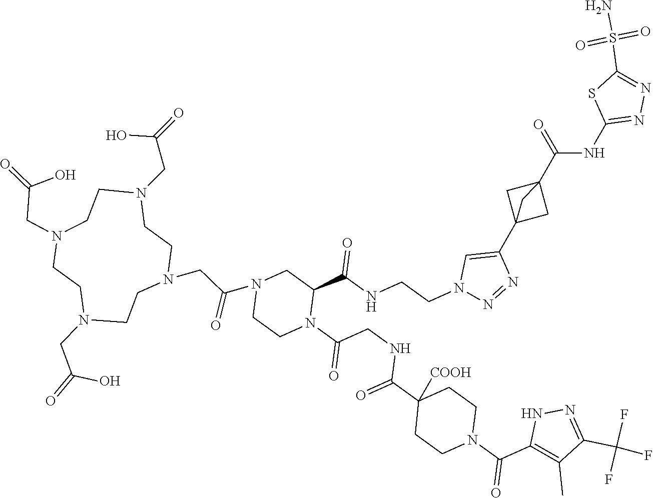 [SMILES: Cc1c(C(F)(F)F)n[nH]c1C(=O)N1CCC(C(=O)O)(C(=O)NCC(=O)N2CCN(C(=O)CN3CCN(CC(=O)O)CCN(CC(=O)O)CCN(CC(=O)O)CC3)C[C@H]2C(=O)NCCn2cc(C34CC(C(=O)Nc5nnc(S(N)(=O)=O)s5)(C3)C4)nn2)CC1] |
| 112.14 | 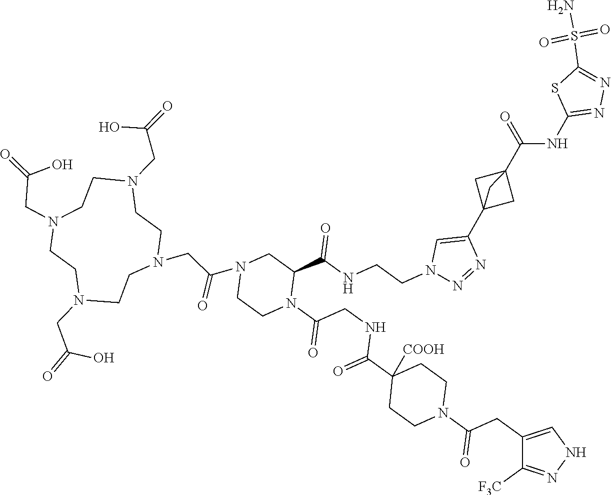 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)CNC(=O)C5(C(=O)O)CCN(C(=O)Cc6c[nH]nc6C(F)(F)F)CC5)nn4)(C2)C3)s1] |
| 112.15 | 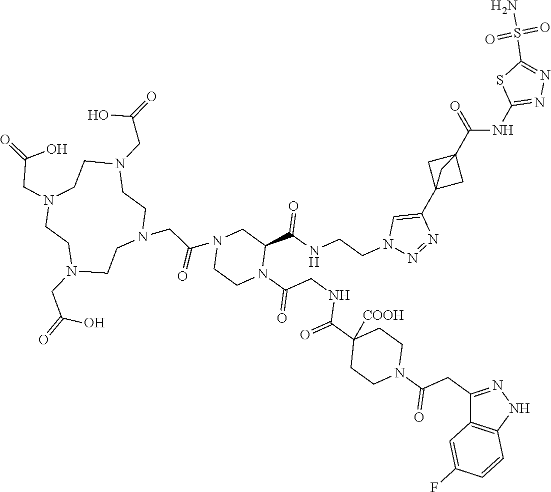 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)CNC(=O)C5(C(=O)O)CCN(C(=O)Cc6n[nH]c7ccc(F)cc67)CC5)nn4)(C2)C3)s1] |
| 112.16 | 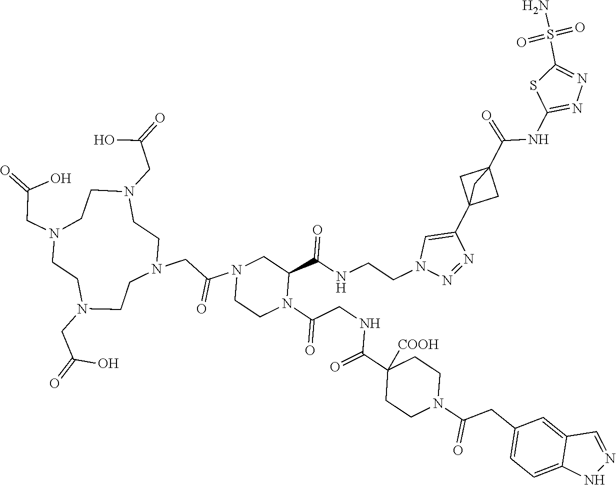 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)CNC(=O)C5(C(=O)O)CCN(C(=O)Cc6ccc7[nH]ncc7c6)CC5)nn4)(C2)C3)s1] |
| 112.17 | 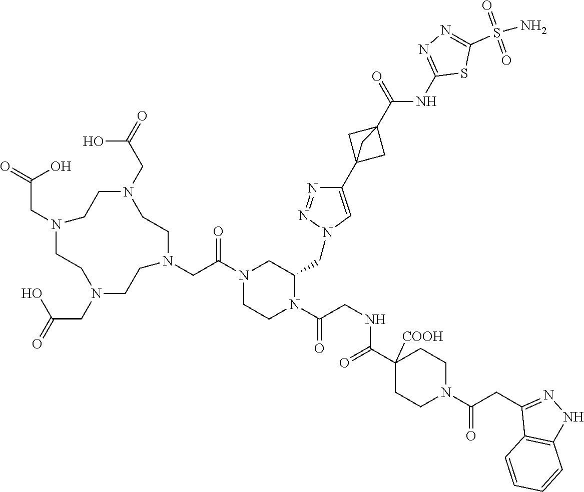 [SMILES: NS(=O)(=O)c1nnc(NC(=O)C23CC(c4cn(C[C@H]5CN(C(=O)CN6CCN(CC(=O)O)CCN(CC(=O)O)CCN(CC(=O)O)CC6)CCN5C(=O)CNC(=O)C5(C(=O)O)CCN(C(=O)Cc6n[nH]c7ccccc67)CC5)nn4)(C2)C3)s1] |
| 112.18 | 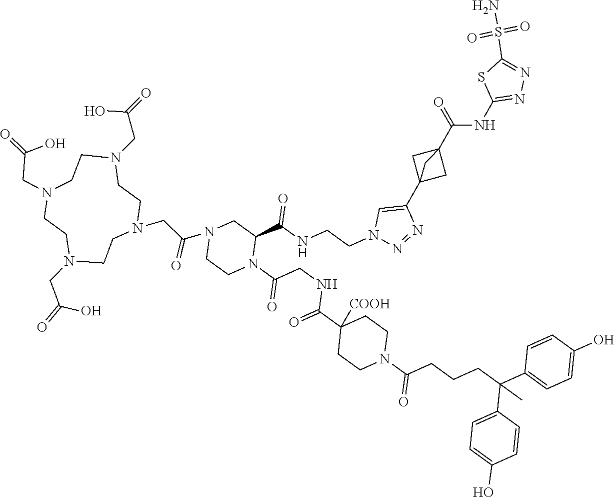 [SMILES: CC(CCCC(=O)N1CCC(C(=O)O)(C(=O)NCC(=O)N2CCN(C(=O)CN3CCN(CC(=O)O)CCN(CC(=O)O)CCN(CC(=O)O)CC3)C[C@H]2C(=O)NCCn2cc(C34CC(C(=O)Nc5nnc(S(N)(=O)=O)s5)(C3)C4)nn2)CC1)(c1ccc(O)cc1)c1ccc(O)cc1] |
| 112.19 | 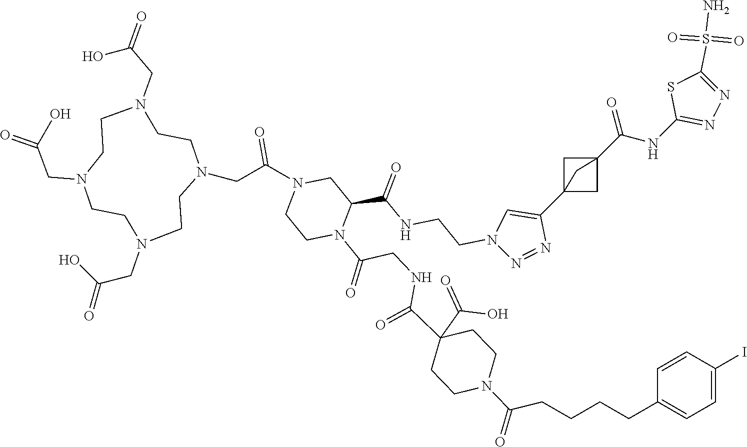 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)CNC(=O)C5(C(=O)O)CCN(C(=O)CCCCc6ccc(I)cc6)CC5)nn4)(C2)C3)s1] |
| 113 | 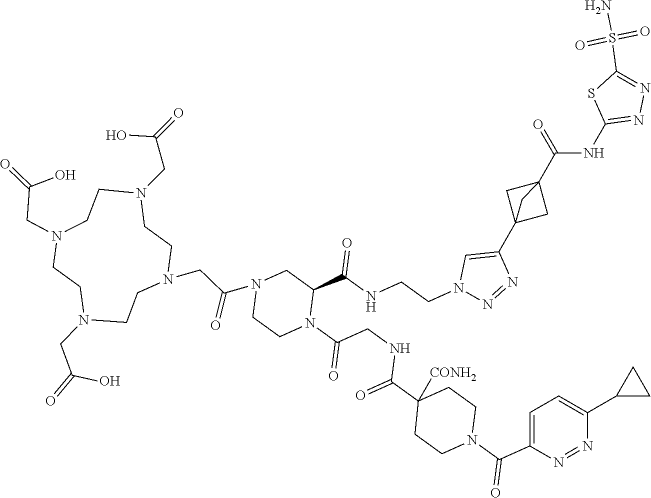 [SMILES: NC(=O)C1(C(=O)NCC(=O)N2CCN(C(=O)CN3CCN(CC(=O)O)CCN(CC(=O)O)CCN(CC(=O)O)CC3)C[C@H]2C(=O)NCCn2cc(C34CC(C(=O)Nc5nnc(S(N)(=O)=O)s5)(C3)C4)nn2)CCN(C(=O)c2ccc(C3CC3)nn2)CC1] |
| 114 | 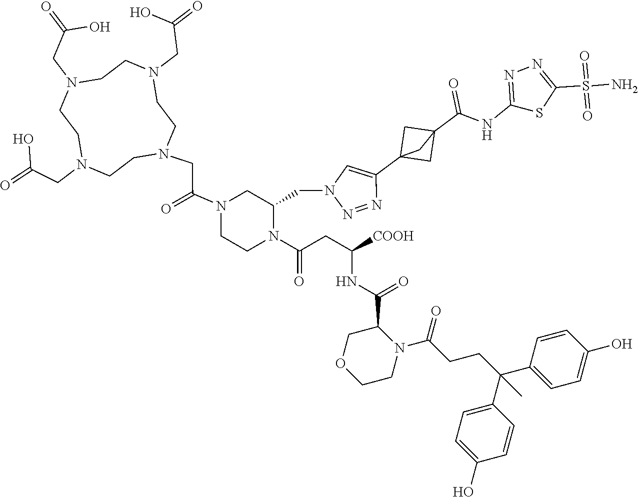 [SMILES: CC(CCC(=O)N1CCOC[C@H]1C(=O)N[C@@H](CC(=O)N1CCN(C(=O)CN2CCN(CC(=O)O)CCN(CC(=O)O)CCN(CC(=O)O)CC2)C[C@@H]1Cn1cc(C23CC(C(=O)Nc4nnc(S(N)(=O)=O)s4)(C2)C3)nn1)C(=O)O)(c1ccc(O)cc1)c1ccc(O)cc1] |
| 114.1 | 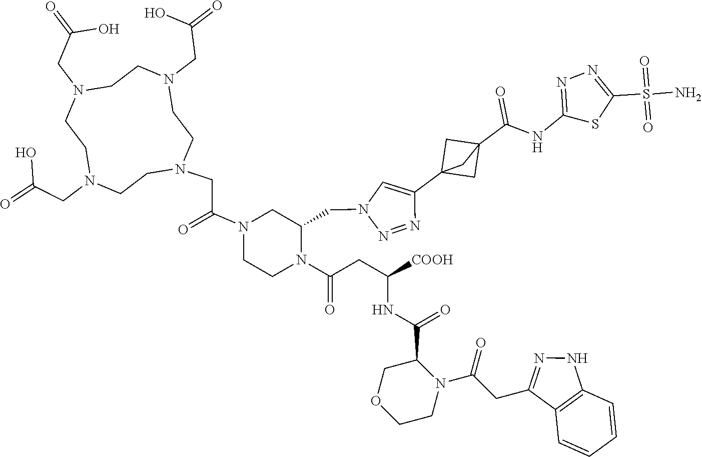 [SMILES: NS(=O)(=O)c1nnc(NC(=O)C23CC(c4cn(C[C@H]5CN(C(=O)CN6CCN(CC(=O)O)CCN(CC(=O)O)CCN(CC(=O)O)CC6)CCN5C(=O)C[C@H](NC(=O)[C@@H]5COCCN5C(=O)Cc5n[nH]c6ccccc56)C(=O)O)nn4)(C2)C3)s1] |
| 114.2 | 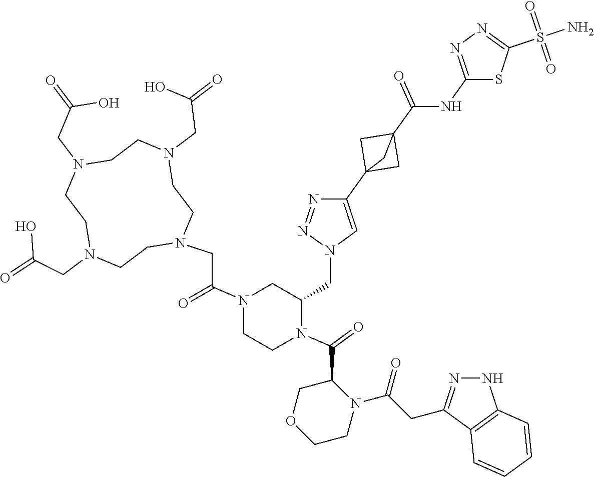 [SMILES: NS(=O)(=O)c1nnc(NC(=O)C23CC(c4cn(C[C@H]5CN(C(=O)CN6CCN(CC(=O)O)CCN(CC(=O)O)CCN(CC(=O)O)CC6)CCN5C(=O)[C@@H]5COCCN5C(=O)Cc5n[nH]c6ccccc56)nn4)(C2)C3)s1] |
| 114.3 | 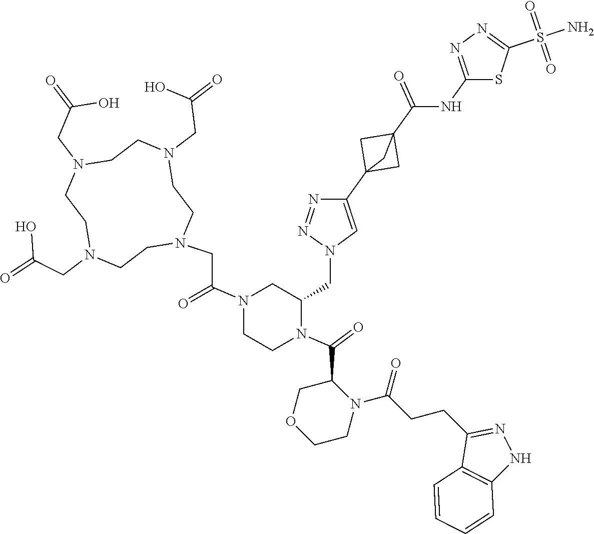 [SMILES: NS(=O)(=O)c1nnc(NC(=O)C23CC(c4cn(C[C@H]5CN(C(=O)CN6CCN(CC(=O)O)CCN(CC(=O)O)CCN(CC(=O)O)CC6)CCN5C(=O)[C@@H]5COCCN5C(=O)CCc5n[nH]c6ccccc56)nn4)(C2)C3)s1] |
| 115 | 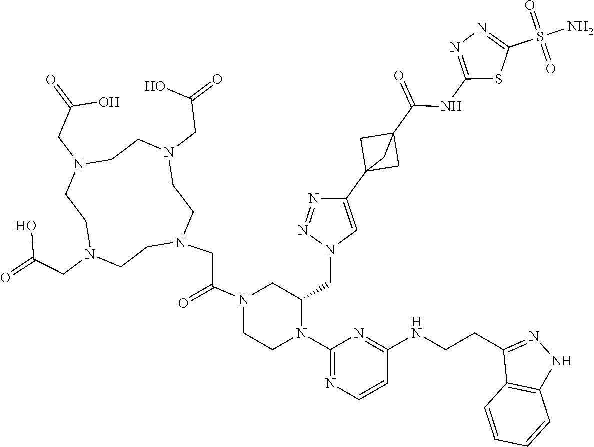 [SMILES: NS(=O)(=O)c1nnc(NC(=O)C23CC(c4cn(C[C@H]5CN(C(=O)CN6CCN(CC(=O)O)CCN(CC(=O)O)CCN(CC(=O)O)CC6)CCN5c5nccc(NCCc6n[nH]c7ccccc67)n5)nn4)(C2)C3)s1] |
| 116 | 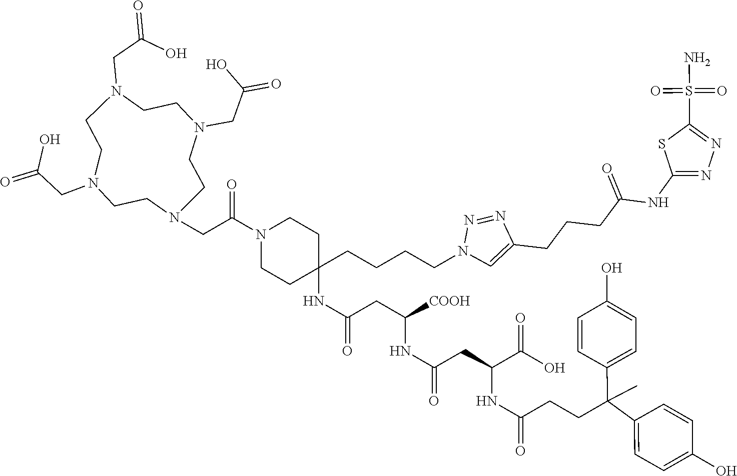 [SMILES: CC(CCC(=O)N[C@@H](CC(=O)N[C@@H](CC(=O)NC1(CCCCn2cc(CCCC(=O)Nc3nnc(S(N)(=O)=O)s3)nn2)CCN(C(=O)CN2CCN(CC(=O)O)CCN(CC(=O)O)CCN(CC(=O)O)CC2)CC1)C(=O)O)C(=O)O)(c1ccc(O)cc1)c1ccc(O)cc1] |
| 116.1 | 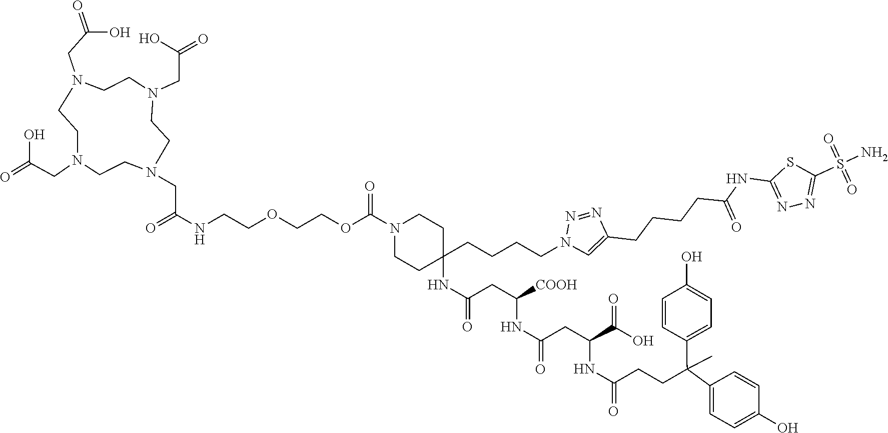 [SMILES: CC(CCC(=O)N[C@@H](CC(=O)N[C@@H](CC(=O)NC1(CCCCn2cc(CCCCC(=O)Nc3nnc(S(N)(=O)=O)s3)nn2)CCN(C(=O)OCCOCCNC(=O)CN2CCN(CC(=O)O)CCN(CC(=O)O)CCN(CC(=O)O)CC2)CC1)C(=O)O)C(=O)O)(c1ccc(O)cc1)c1ccc(O)cc1] |
| 116.2 | 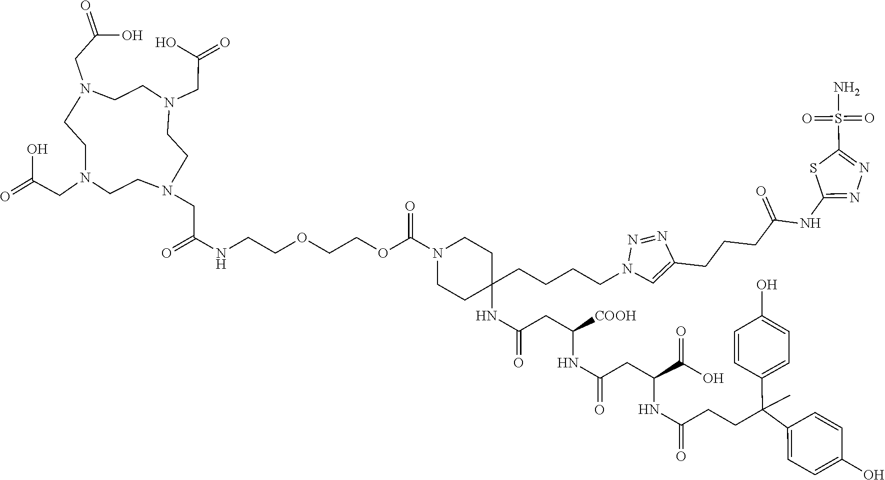 [SMILES: CC(CCC(=O)N[C@@H](CC(=O)N[C@@H](CC(=O)NC1(CCCCn2cc(CCCC(=O)Nc3nnc(S(N)(=O)=O)s3)nn2)CCN(C(=O)OCCOCCNC(=O)CN2CCN(CC(=O)O)CCN(CC(=O)O)CCN(CC(=O)O)CC2)CC1)C(=O)O)C(=O)O)(c1ccc(O)cc1)c1ccc(O)cc1] |
| 116.3 | 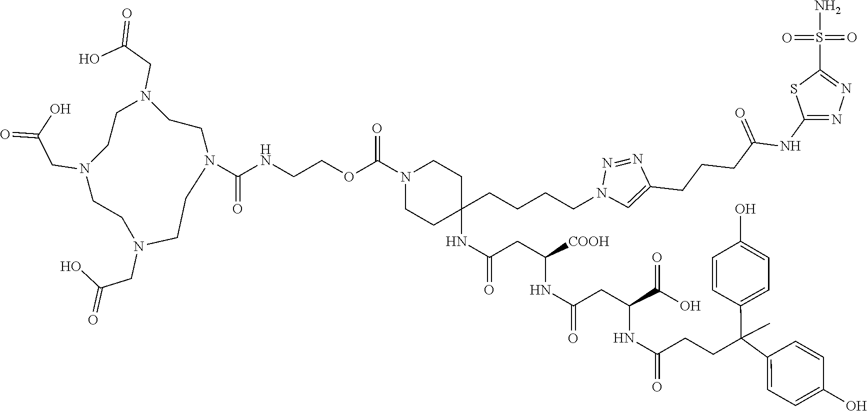 [SMILES: CC(CCC(=O)N[C@@H](CC(=O)N[C@@H](CC(=O)NC1(CCCCn2cc(CCCC(=O)Nc3nnc(S(N)(=O)=O)s3)nn2)CCN(C(=O)OCCNC(=O)N2CCN(CC(=O)O)CCN(CC(=O)O)CCN(CC(=O)O)CC2)CC1)C(=O)O)C(=O)O)(c1ccc(O)cc1)c1ccc(O)cc1] |
| 116.4 | 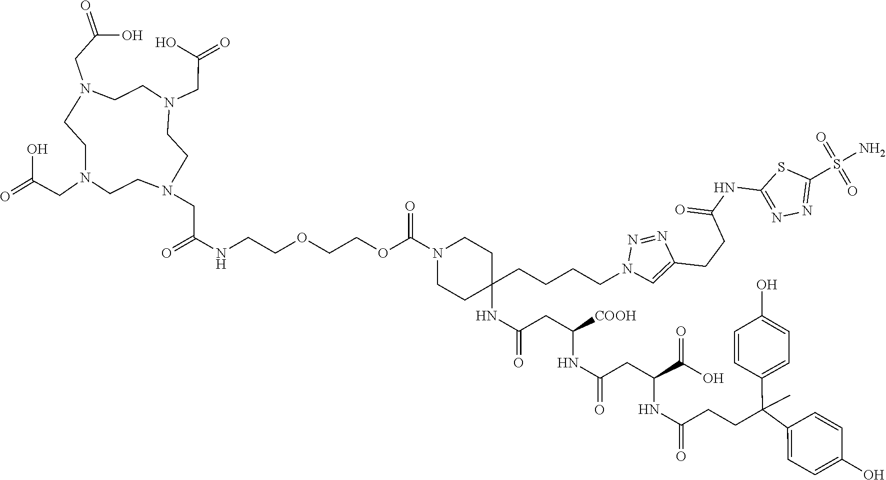 [SMILES: CC(CCC(=O)N[C@@H](CC(=O)N[C@@H](CC(=O)NC1(CCCCn2cc(CCC(=O)Nc3nnc(S(N)(=O)=O)s3)nn2)CCN(C(=O)OCCOCCNC(=O)CN2CCN(CC(=O)O)CCN(CC(=O)O)CCN(CC(=O)O)CC2)CC1)C(=O)O)C(=O)O)(c1ccc(O)cc1)c1ccc(O)cc1] |
| 117 | 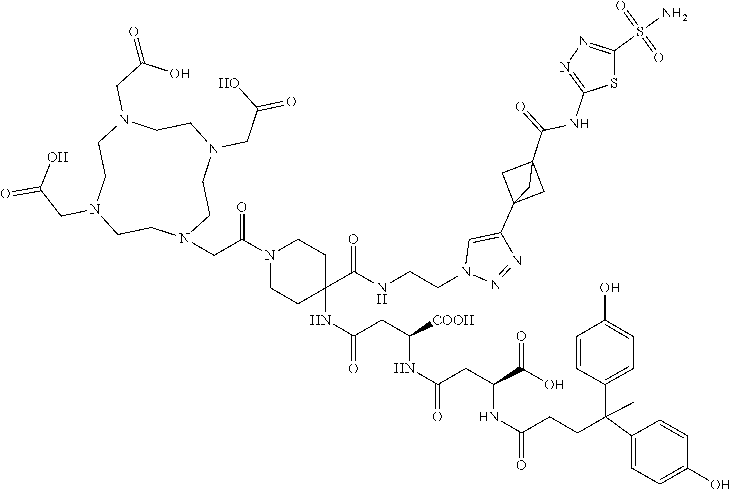 [SMILES: CC(CCC(=O)N[C@@H](CC(=O)N[C@@H](CC(=O)NC1(C(=O)NCCn2cc(C34CC(C(=O)Nc5nnc(S(N)(=O)=O)s5)(C3)C4)nn2)CCN(C(=O)CN2CCN(CC(=O)O)CCN(CC(=O)O)CCN(CC(=O)O)CC2)CC1)C(=O)O)C(=O)O)(c1ccc(O)cc1)c1ccc(O)cc1] |
| 117.1 | 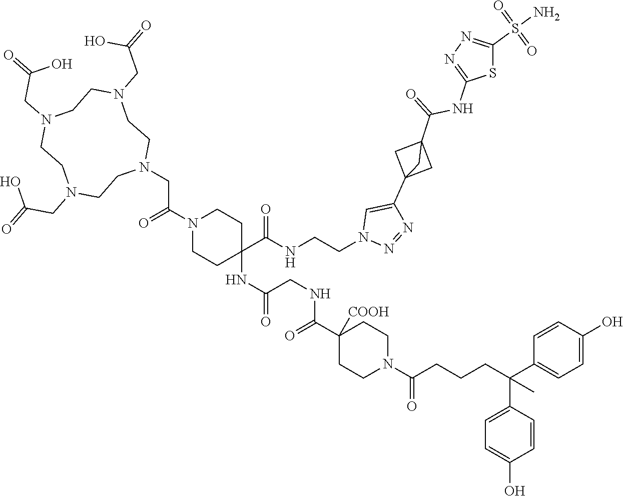 [SMILES: CC(CCCC(=O)N1CCC(C(=O)O)(C(=O)NCC(=O)NC2(C(=O)NCCn3cc(C45CC(C(=O)Nc6nnc(S(N)(=O)=O)s6)(C4)C5)nn3)CCN(C(=O)CN3CCN(CC(=O)O)CCN(CC(=O)O)CCN(CC(=O)O)CC3)CC2)CC1)(c1ccc(O)cc1)c1ccc(O)cc1] |
| 117.2 | 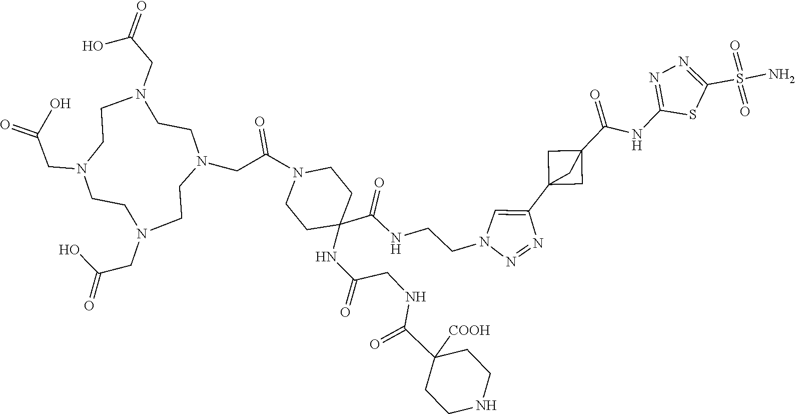 [SMILES: NS(=O)(=O)c1nnc(NC(=O)C23CC(c4cn(CCNC(=O)C5(NC(=O)CNC(=O)C6(C(=O)O)CCNCC6)CCN(C(=O)CN6CCN(CC(=O)O)CCN(CC(=O)O)CCN(CC(=O)O)CC6)CC5)nn4)(C2)C3)s1] |
| 117.3 | 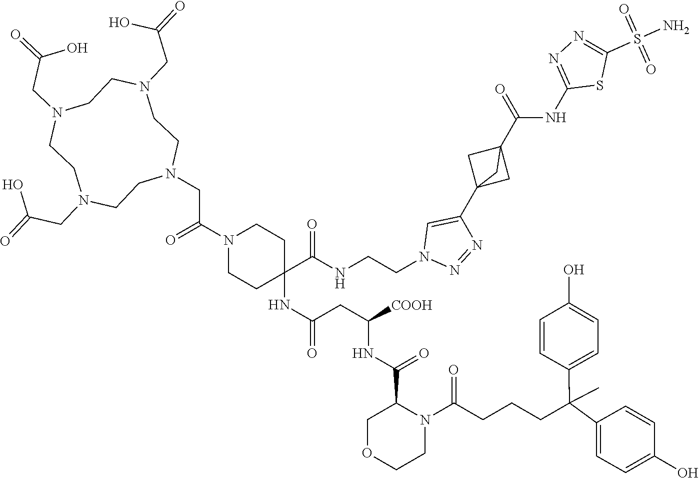 [SMILES: CC(CCCC(=O)N1CCOC[C@H]1C(=O)N[C@@H](CC(=O)NC1(C(=O)NCCn2cc(C34CC(C(=O)Nc5nnc(S(N)(=O)=O)s5)(C3)C4)nn2)CCN(C(=O)CN2CCN(CC(=O)O)CCN(CC(=O)O)CCN(CC(=O)O)CC2)CC1)C(=O)O)(c1ccc(O)cc1)c1ccc(O)cc1] |
| 117.4 | 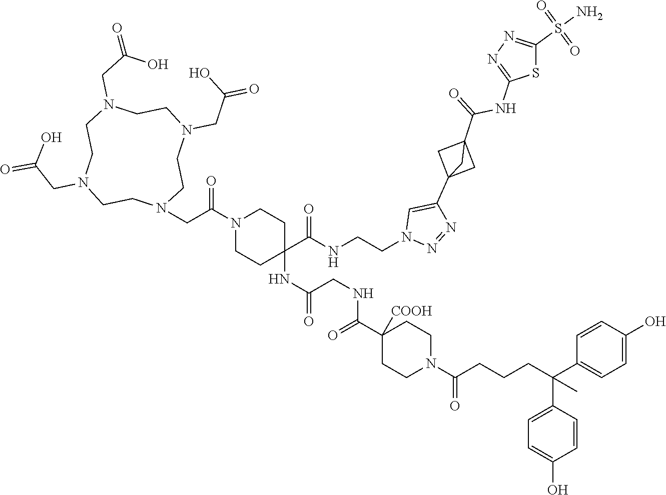 [SMILES: CC(CCCC(=O)N1CCC(C(=O)O)(C(=O)NCC(=O)NC2(C(=O)NCCn3cc(C45CC(C(=O)Nc6nnc(S(N)(=O)=O)s6)(C4)C5)nn3)CCN(C(=O)CN3CCN(CC(=O)O)CCN(CC(=O)O)CCN(CC(=O)O)CC3)CC2)CC1)(c1ccc(O)cc1)c1ccc(O)cc1] |
| 117.5 | 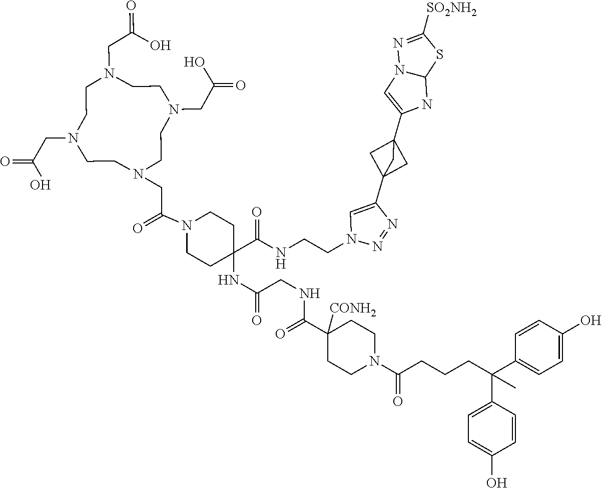 [SMILES: CC(CCCC(=O)N1CCC(C(N)=O)(C(=O)NCC(=O)NC2(C(=O)NCCn3cc(C45CC(C6=CN7N=C(S(N)(=O)=O)SC7N6)(C4)C5)nn3)CCN(C(=O)CN3CCN(CC(=O)O)CCN(CC(=O)O)CCN(CC(=O)O)CC3)CC2)CC1)(c1ccc(O)cc1)c1ccc(O)cc1] |
| 117.6 | 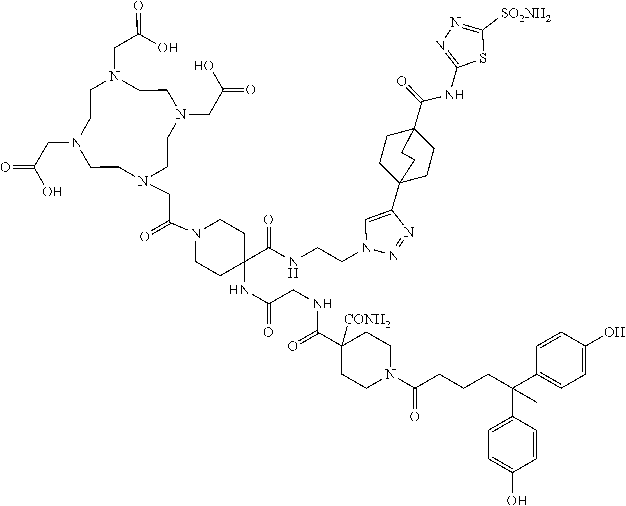 [SMILES: CC(CCCC(=O)N1CCC(C(N)=O)(C(=O)NCC(=O)NC2(C(=O)NCCn3cc(C45CCC(C(=O)Nc6nnc(S(N)(=O)=O)s6)(CC4)CC5)nn3)CCN(C(=O)CN3CCN(CC(=O)O)CCN(CC(=O)O)CCN(CC(=O)O)CC3)CC2)CC1)(c1ccc(O)cc1)c1ccc(O)cc1] |
| 117.7 | 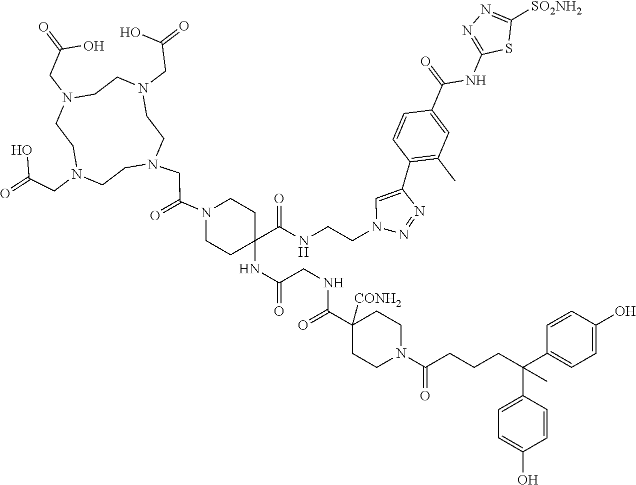 [SMILES: Cc1cc(C(=O)Nc2nnc(S(N)(=O)=O)s2)ccc1-c1cn(CCNC(=O)C2(NC(=O)CNC(=O)C3(C(N)=O)CCN(C(=O)CCCC(C)(c4ccc(O)cc4)c4ccc(O)cc4)CC3)CCN(C(=O)CN3CCN(CC(=O)O)CCN(CC(=O)O)CCN(CC(=O)O)CC3)CC2)nn1] |
| 117.8 | 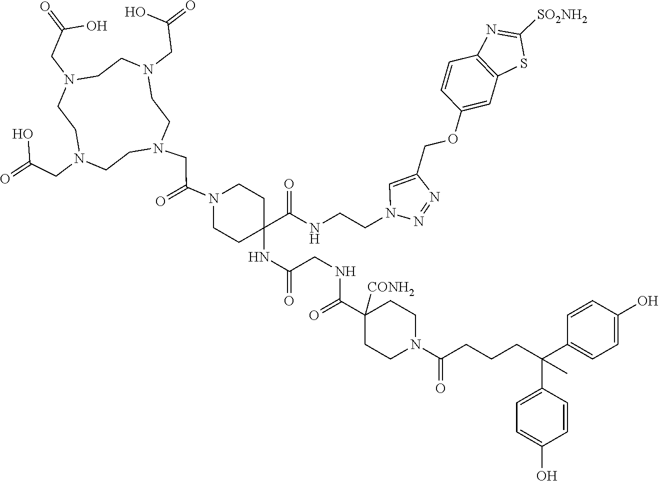 [SMILES: CC(CCCC(=O)N1CCC(C(N)=O)(C(=O)NCC(=O)NC2(C(=O)NCCn3cc(COc4ccc5nc(S(N)(=O)=O)sc5c4)nn3)CCN(C(=O)CN3CCN(CC(=O)O)CCN(CC(=O)O)CCN(CC(=O)O)CC3)CC2)CC1)(c1ccc(O)cc1)c1ccc(O)cc1] |
| 118 | 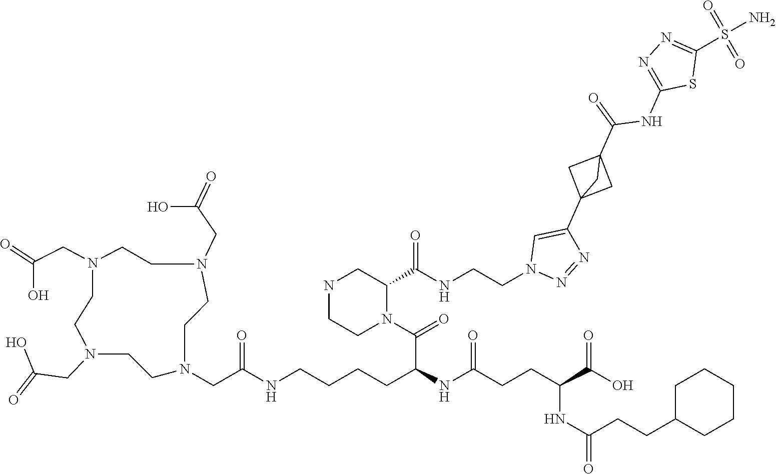 [SMILES: NS(=O)(=O)c1nnc(NC(=O)C23CC(c4cn(CCNC(=O)[C@H]5CNCCN5C(=O)[C@H](CCCCNC(=O)CN5CCN(CC(=O)O)CCN(CC(=O)O)CCN(CC(=O)O)CC5)NC(=O)CC[C@H](NC(=O)CCC5CCCCC5)C(=O)O)nn4)(C2)C3)s1] |
| 118.1 | 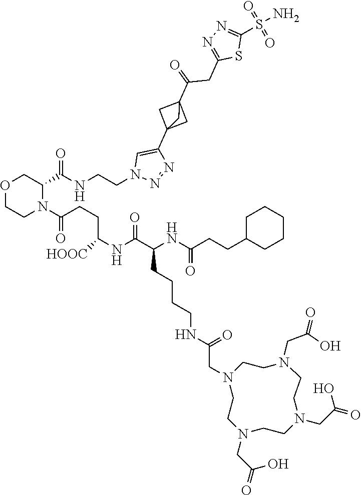 [SMILES: NS(=O)(=O)c1nnc(CC(=O)C23CC(c4cn(CCNC(=O)[C@H]5COCCN5C(=O)CC[C@H](NC(=O)[C@H](CCCCNC(=O)CN5CCN(CC(=O)O)CCN(CC(=O)O)CCN(CC(=O)O)CC5)NC(=O)CCC5CCCCC5)C(=O)O)nn4)(C2)C3)s1] |
| 118.2 | 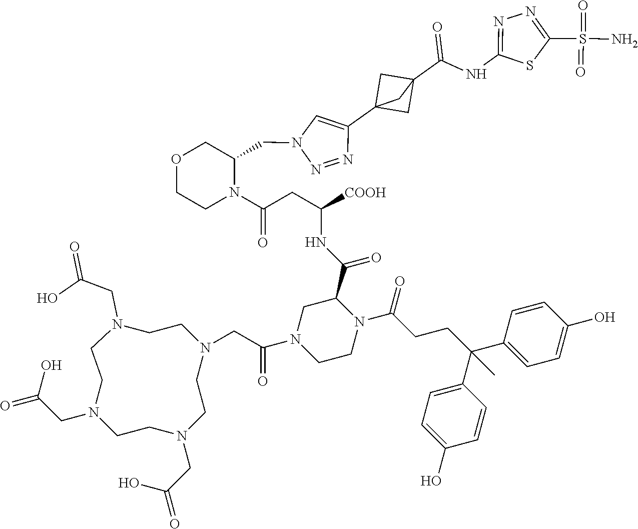 [SMILES: CC(CCC(=O)N1CCN(C(=O)CN2CCN(CC(=O)O)CCN(CC(=O)O)CCN(CC(=O)O)CC2)C[C@H]1C(=O)N[C@@H](CC(=O)N1CCOC[C@@H]1Cn1cc(C23CC(C(=O)Nc4nnc(S(N)(=O)=O)s4)(C2)C3)nn1)C(=O)O)(c1ccc(O)cc1)c1ccc(O)cc1] |
| 118.3 | 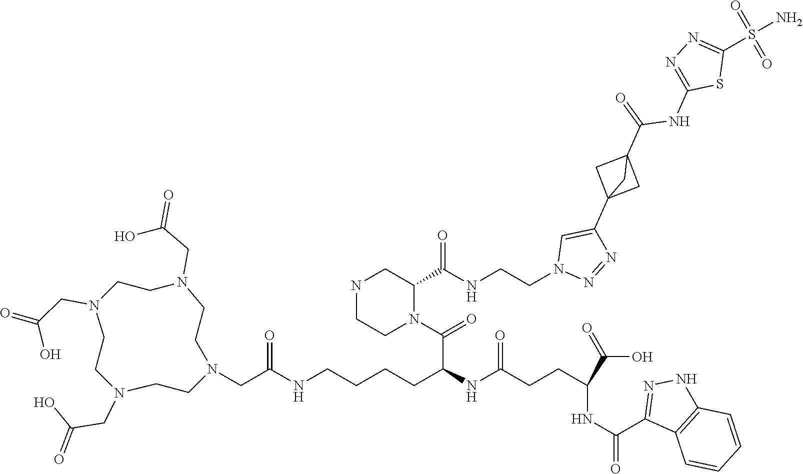 [SMILES: NS(=O)(=O)c1nnc(NC(=O)C23CC(c4cn(CCNC(=O)[C@H]5CNCCN5C(=O)[C@H](CCCCNC(=O)CN5CCN(CC(=O)O)CCN(CC(=O)O)CCN(CC(=O)O)CC5)NC(=O)CC[C@H](NC(=O)c5n[nH]c6ccccc56)C(=O)O)nn4)(C2)C3)s1] |
| 118.4 | 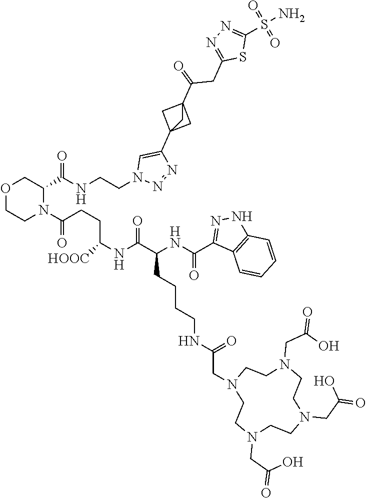 [SMILES: NS(=O)(=O)c1nnc(CC(=O)C23CC(c4cn(CCNC(=O)[C@H]5COCCN5C(=O)CC[C@H](NC(=O)[C@H](CCCCNC(=O)CN5CCN(CC(=O)O)CCN(CC(=O)O)CCN(CC(=O)O)CC5)NC(=O)c5n[nH]c6ccccc56)C(=O)O)nn4)(C2)C3)s1] |
| 119 | 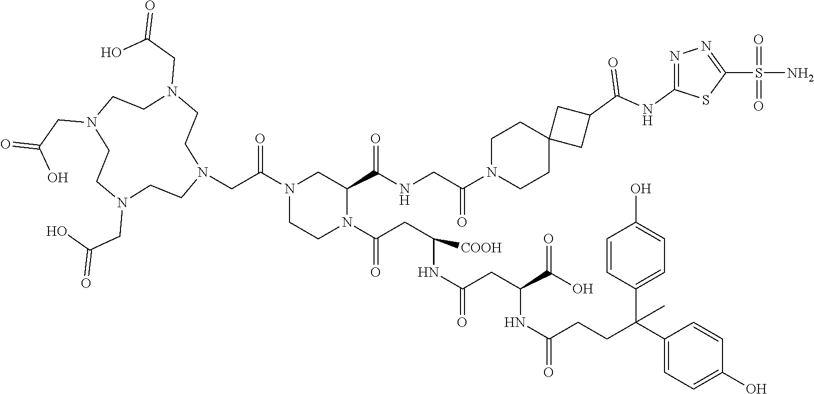 [SMILES: CC(CCC(=O)N[C@@H](CC(=O)N[C@@H](CC(=O)N1CCN(C(=O)CN2CCN(CC(=O)O)CCN(CC(=O)O)CCN(CC(=O)O)CC2)C[C@H]1C(=O)NCC(=O)N1CCC2(CC1)CC(C(=O)Nc1nnc(S(N)(=O)=O)s1)C2)C(=O)O)C(=O)O)(c1ccc(O)cc1)c1ccc(O)cc1] |
| 120 | 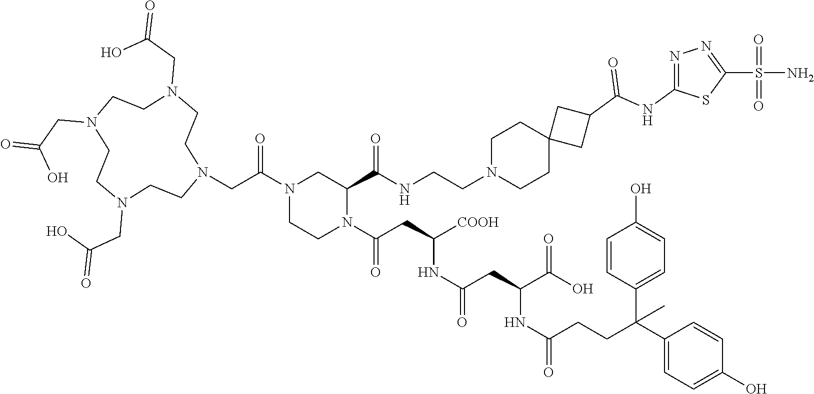 [SMILES: CC(CCC(=O)N[C@@H](CC(=O)N[C@@H](CC(=O)N1CCN(C(=O)CN2CCN(CC(=O)O)CCN(CC(=O)O)CCN(CC(=O)O)CC2)C[C@H]1C(=O)NCCN1CCC2(CC1)CC(C(=O)Nc1nnc(S(N)(=O)=O)s1)C2)C(=O)O)C(=O)O)(c1ccc(O)cc1)c1ccc(O)cc1] |
| 121 | 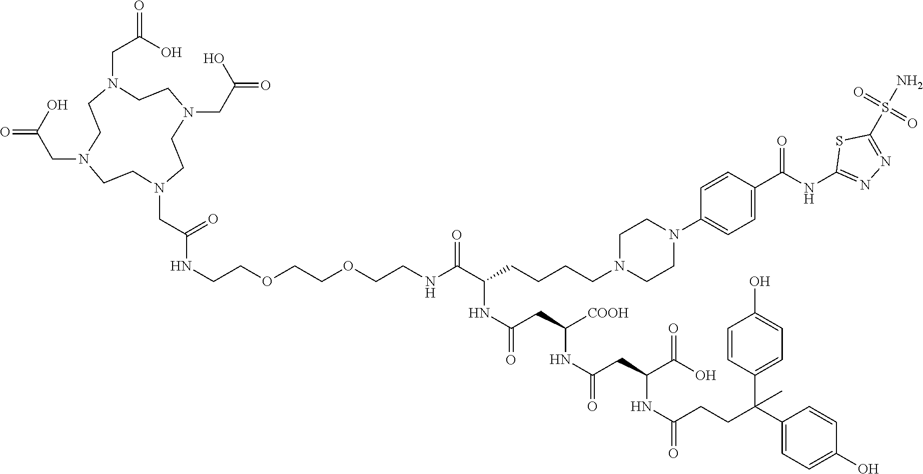 [SMILES: CC(CCC(=O)N[C@@H](CC(=O)N[C@@H](CC(=O)N[C@@H](CCCCN1CCN(c2ccc(C(=O)Nc3nnc(S(N)(=O)=O)s3)cc2)CC1)C(=O)NCCOCCOCCNC(=O)CN1CCN(CC(=O)O)CCN(CC(=O)O)CCN(CC(=O)O)CC1)C(=O)O)C(=O)O)(c1ccc(O)cc1)c1ccc(O)cc1] |
| 121.1 | 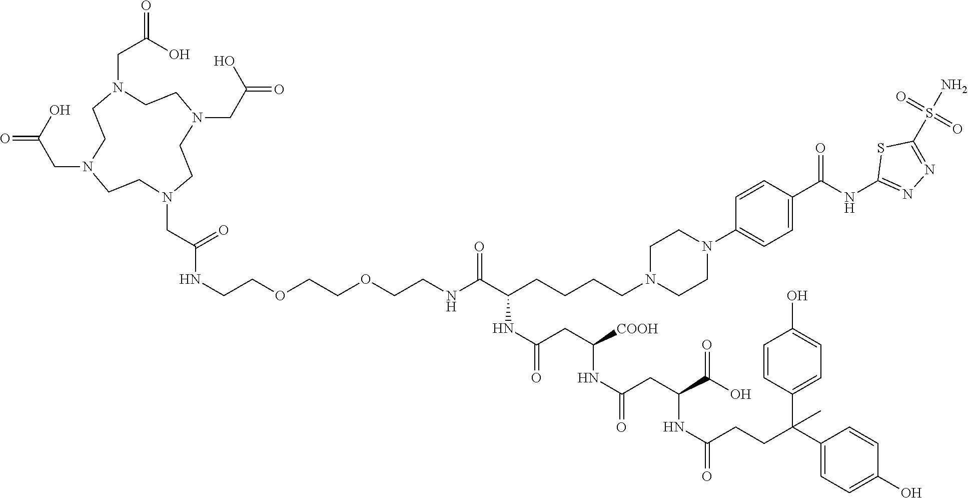 [SMILES: CC(CCC(=O)N[C@@H](CC(=O)N[C@@H](CC(=O)N[C@@H](CCCCN1CCN(c2ccc(C(=O)Nc3nnc(S(N)(=O)=O)s3)cc2)CC1)C(=O)NCCOCCOCCNC(=O)CN1CCN(CC(=O)O)CCN(CC(=O)O)CCN(CC(=O)O)CC1)C(=O)O)C(=O)O)(c1ccc(O)cc1)c1ccc(O)cc1] |
| 122 | 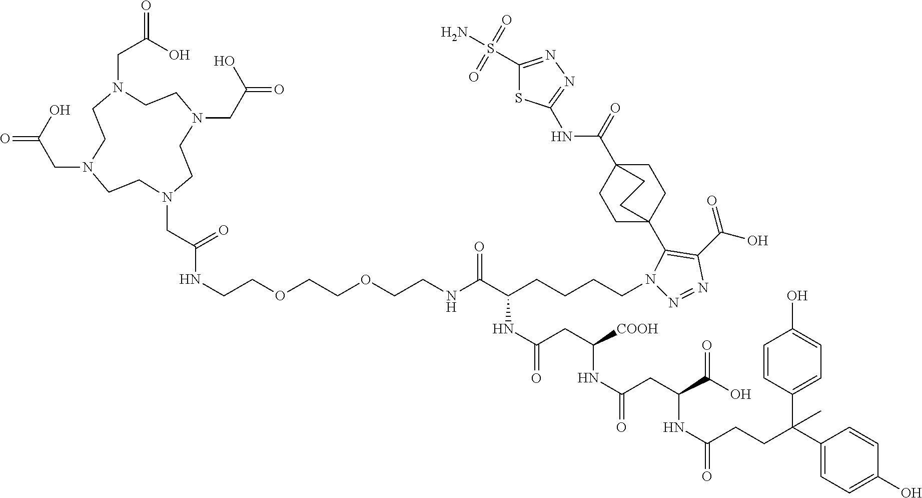 [SMILES: CC(CCC(=O)N[C@@H](CC(=O)N[C@@H](CC(=O)N[C@@H](CCCCn1nnc(C(=O)O)c1C12CCC(C(=O)Nc3nnc(S(N)(=O)=O)s3)(CC1)CC2)C(=O)NCCOCCOCCNC(=O)CN1CCN(CC(=O)O)CCN(CC(=O)O)CCN(CC(=O)O)CC1)C(=O)O)C(=O)O)(c1ccc(O)cc1)c1ccc(O)cc1] |
| 123 | 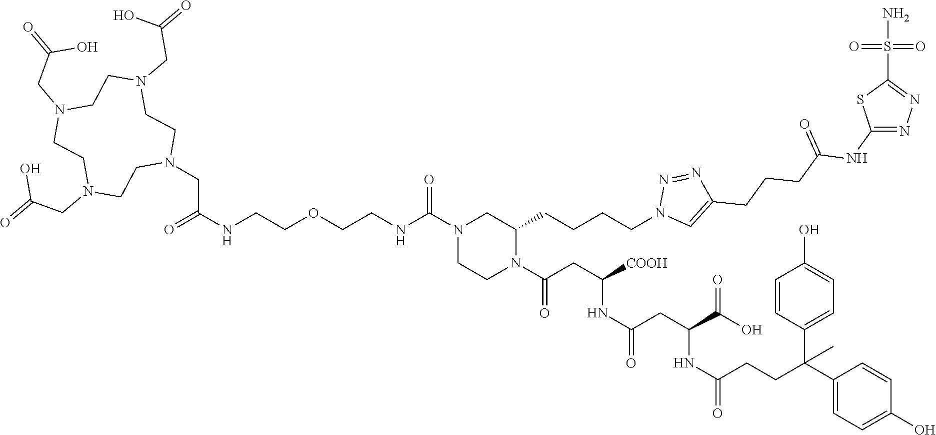 [SMILES: CC(CCC(=O)N[C@@H](CC(=O)N[C@@H](CC(=O)N1CCN(C(=O)NCCOCCNC(=O)CN2CCN(CC(=O)O)CCN(CC(=O)O)CCN(CC(=O)O)CC2)C[C@@H]1CCCCn1cc(CCCC(=O)Nc2nnc(S(N)(=O)=O)s2)nn1)C(=O)O)C(=O)O)(c1ccc(O)cc1)c1ccc(O)cc1] |
| 124 | 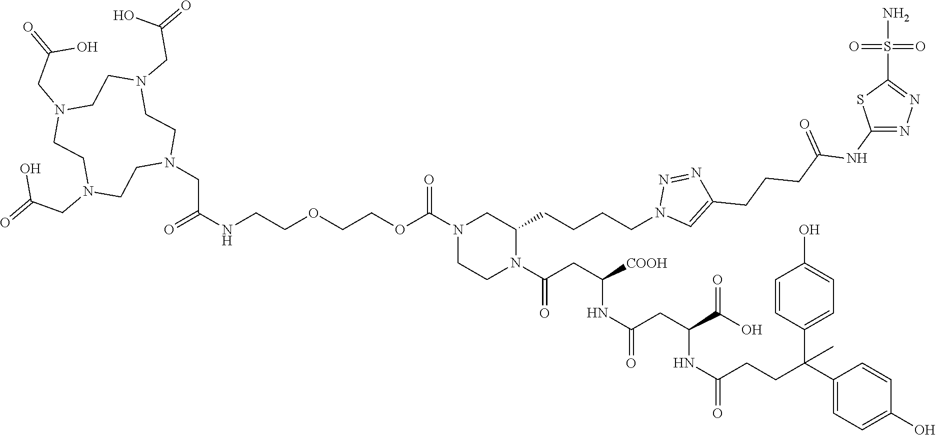 [SMILES: CC(CCC(=O)N[C@@H](CC(=O)N[C@@H](CC(=O)N1CCN(C(=O)OCCOCCNC(=O)CN2CCN(CC(=O)O)CCN(CC(=O)O)CCN(CC(=O)O)CC2)C[C@@H]1CCCCn1cc(CCCC(=O)Nc2nnc(S(N)(=O)=O)s2)nn1)C(=O)O)C(=O)O)(c1ccc(O)cc1)c1ccc(O)cc1] |
| 125 | 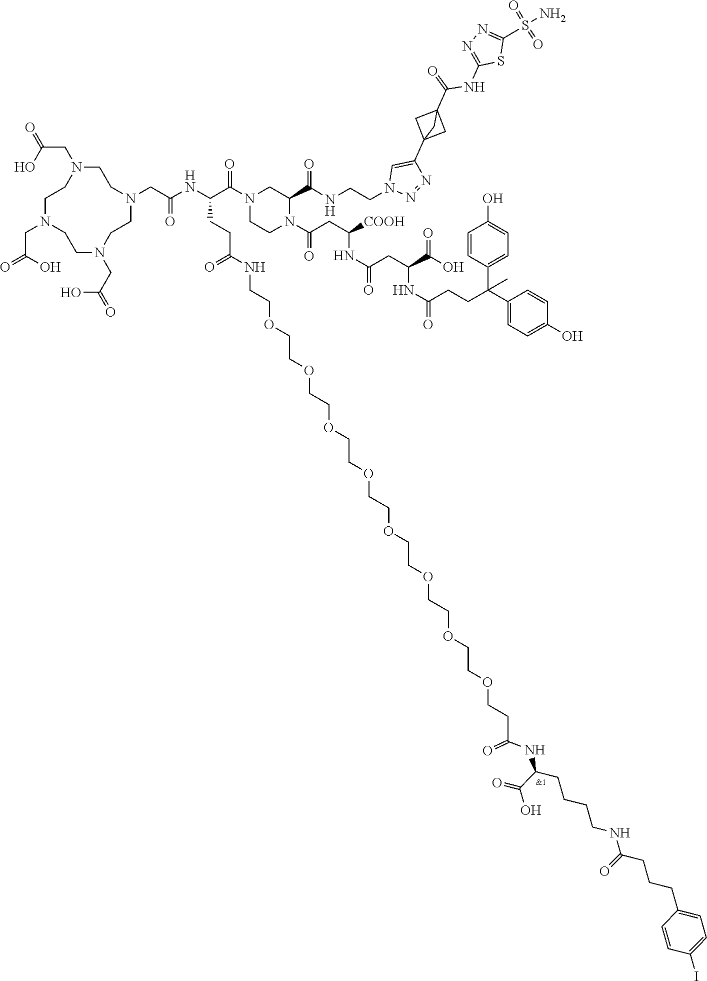 [SMILES: CC(CCC(=O)N[C@@H](CC(=O)N[C@@H](CC(=O)N1CCN(C(=O)[C@H](CCC(=O)NCCOCCOCCOCCOCCOCCOCCOCCOCCC(=O)N[C@@H](CCCCNC(=O)CCCc2ccc(I)cc2)C(=O)O)NC(=O)CN2CCN(CC(=O)O)CCN(CC(=O)O)CCN(CC(=O)O)CC2)C[C@H]1C(=O)NCCn1cc(C23CC(C(=O)Nc4nnc(S(N)(=O)=O)s4)(C2)C3)nn1)C(=O)O)C(=O)O)(c1ccc(O)cc1)c1ccc(O)cc1] |
| 125.1 | 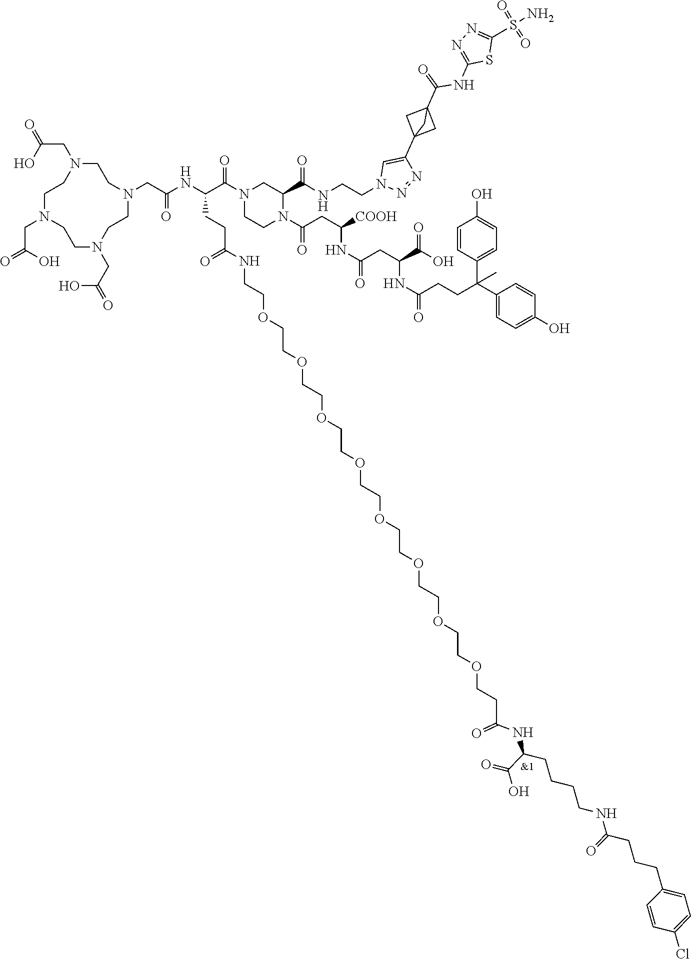 [SMILES: CC(CCC(=O)N[C@@H](CC(=O)N[C@@H](CC(=O)N1CCN(C(=O)[C@H](CCC(=O)NCCOCCOCCOCCOCCOCCOCCOCCOCCC(=O)N[C@@H](CCCCNC(=O)CCCc2ccc(Cl)cc2)C(=O)O)NC(=O)CN2CCN(CC(=O)O)CCN(CC(=O)O)CCN(CC(=O)O)CC2)C[C@H]1C(=O)NCCn1cc(C23CC(C(=O)Nc4nnc(S(N)(=O)=O)s4)(C2)C3)nn1)C(=O)O)C(=O)O)(c1ccc(O)cc1)c1ccc(O)cc1] |
| 126 | 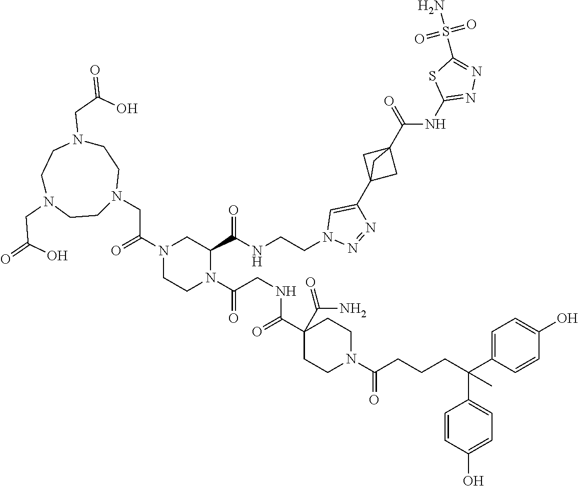 [SMILES: CC(CCCC(=O)N1CCC(C(N)=O)(C(=O)NCC(=O)N2CCN(C(=O)CN3CCN(CC(=O)O)CCN(CC(=O)O)CC3)C[C@H]2C(=O)NCCn2cc(C34CC(C(=O)Nc5nnc(S(N)(=O)=O)s5)(C3)C4)nn2)CC1)(c1ccc(O)cc1)c1ccc(O)cc1] |
| 126.1 | 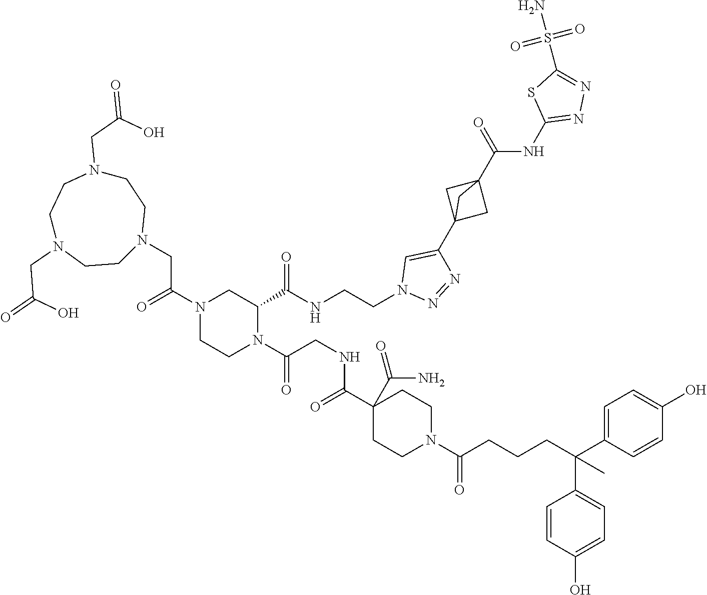 [SMILES: CC(CCCC(=O)N1CCC(C(N)=O)(C(=O)NCC(=O)N2CCN(C(=O)CN3CCN(CC(=O)O)CCN(CC(=O)O)CC3)C[C@@H]2C(=O)NCCn2cc(C34CC(C(=O)Nc5nnc(S(N)(=O)=O)s5)(C3)C4)nn2)CC1)(c1ccc(O)cc1)c1ccc(O)cc1] |
| 126.2 | 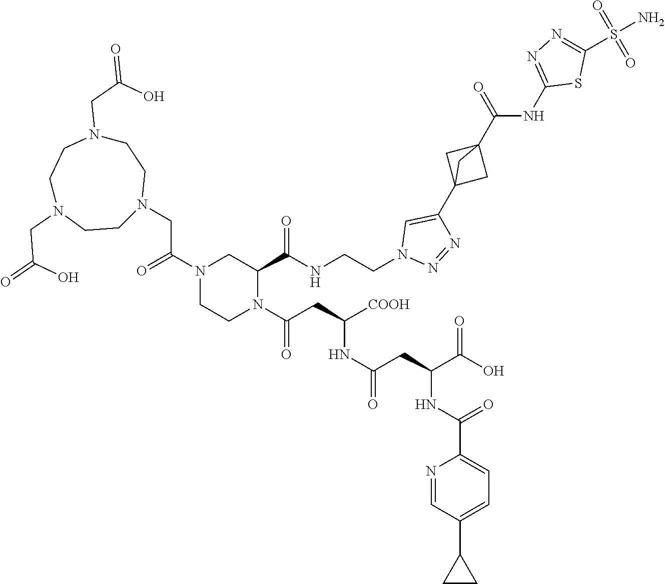 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CC6)CCN5C(=O)C[C@H](NC(=O)C[C@H](NC(=O)c5ccc(C6CC6)cn5)C(=O)O)C(=O)O)nn4)(C2)C3)s1] |
| 126.3 | 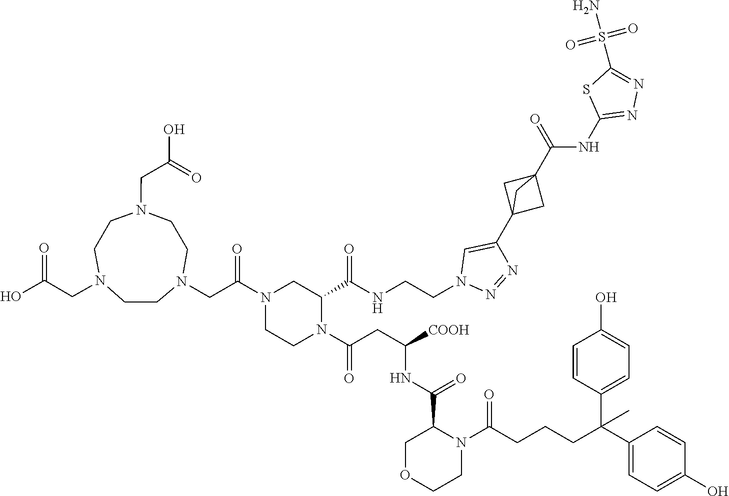 [SMILES: CC(CCCC(=O)N1CCOC[C@H]1C(=O)N[C@@H](CC(=O)N1CCN(C(=O)CN2CCN(CC(=O)O)CCN(CC(=O)O)CC2)C[C@@H]1C(=O)NCCn1cc(C23CC(C(=O)Nc4nnc(S(N)(=O)=O)s4)(C2)C3)nn1)C(=O)O)(c1ccc(O)cc1)c1ccc(O)cc1] |
| 126.4 | 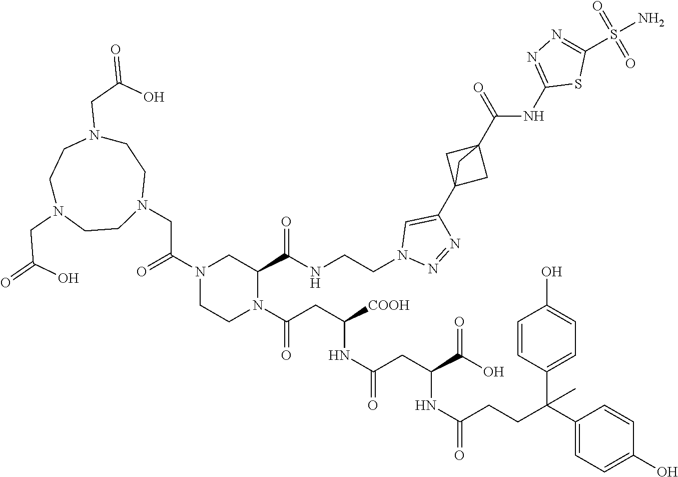 [SMILES: CC(CCC(=O)N[C@@H](CC(=O)N[C@@H](CC(=O)N1CCN(C(=O)CN2CCN(CC(=O)O)CCN(CC(=O)O)CC2)C[C@H]1C(=O)NCCn1cc(C23CC(C(=O)Nc4nnc(S(N)(=O)=O)s4)(C2)C3)nn1)C(=O)O)C(=O)O)(c1ccc(O)cc1)c1ccc(O)cc1] |
| 126.5 | 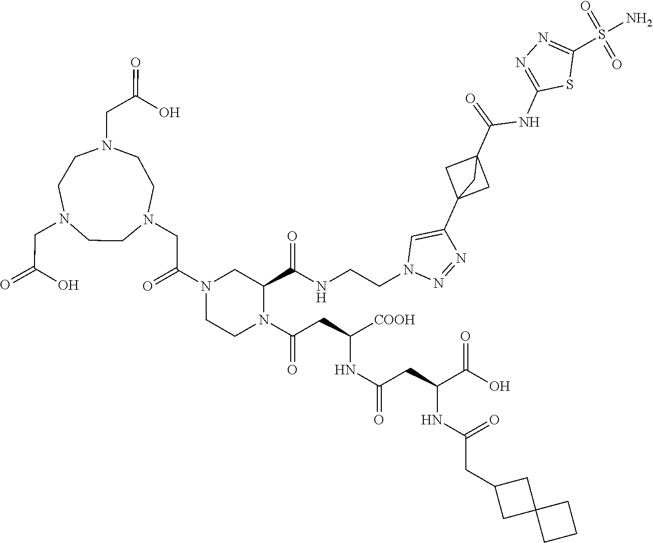 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CC6)CCN5C(=O)C[C@H](NC(=O)C[C@H](NC(=O)CC5CC6(CCC6)C5)C(=O)O)C(=O)O)nn4)(C2)C3)s1] |
| 127 | 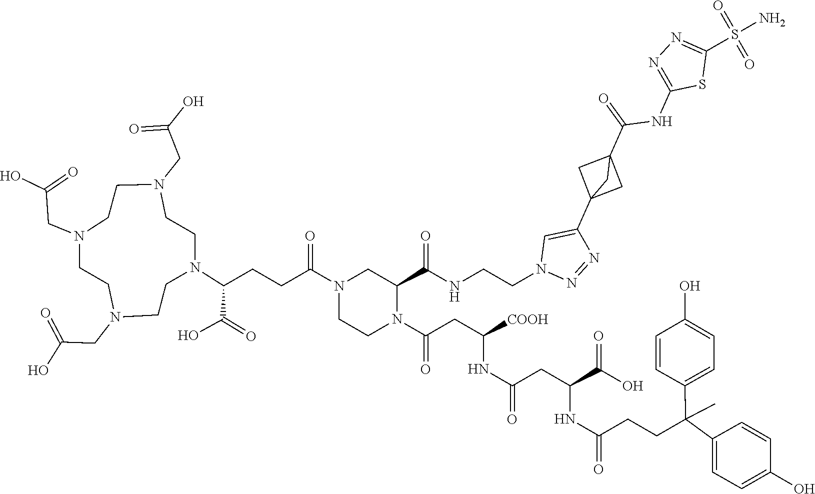 [SMILES: CC(CCC(=O)N[C@@H](CC(=O)N[C@@H](CC(=O)N1CCN(C(=O)CC[C@H](C(=O)O)N2CCN(CC(=O)O)CCN(CC(=O)O)CCN(CC(=O)O)CC2)C[C@H]1C(=O)NCCn1cc(C23CC(C(=O)Nc4nnc(S(N)(=O)=O)s4)(C2)C3)nn1)C(=O)O)C(=O)O)(c1ccc(O)cc1)c1ccc(O)cc1] |
| 127.1 | 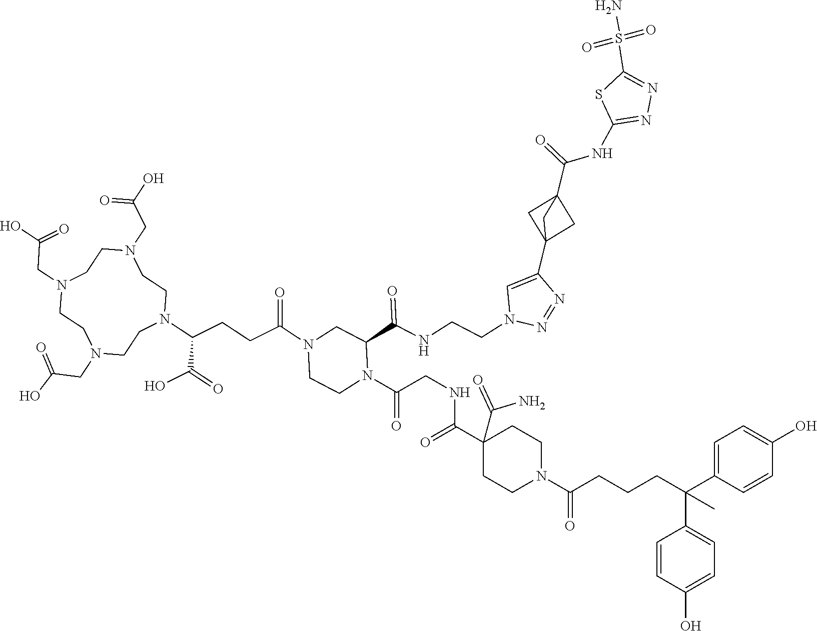 [SMILES: CC(CCCC(=O)N1CCC(C(N)=O)(C(=O)NCC(=O)N2CCN(C(=O)CC[C@H](C(=O)O)N3CCN(CC(=O)O)CCN(CC(=O)O)CCN(CC(=O)O)CC3)C[C@H]2C(=O)NCCn2cc(C34CC(C(=O)Nc5nnc(S(N)(=O)=O)s5)(C3)C4)nn2)CC1)(c1ccc(O)cc1)c1ccc(O)cc1] |
| 127.2 | 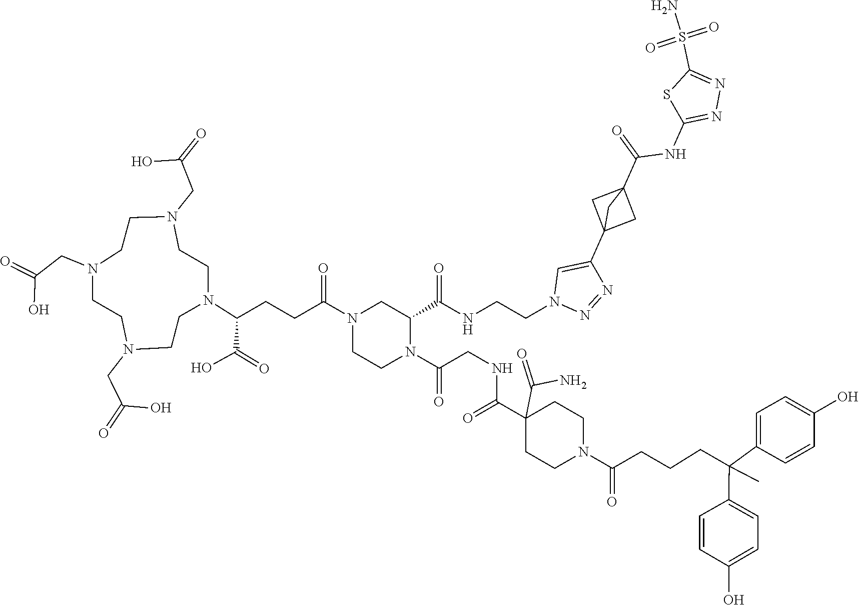 [SMILES: CC(CCCC(=O)N1CCC(C(N)=O)(C(=O)NCC(=O)N2CCN(C(=O)CC[C@H](C(=O)O)N3CCN(CC(=O)O)CCN(CC(=O)O)CCN(CC(=O)O)CC3)C[C@@H]2C(=O)NCCn2cc(C34CC(C(=O)Nc5nnc(S(N)(=O)=O)s5)(C3)C4)nn2)CC1)(c1ccc(O)cc1)c1ccc(O)cc1] |
| 127.3 | 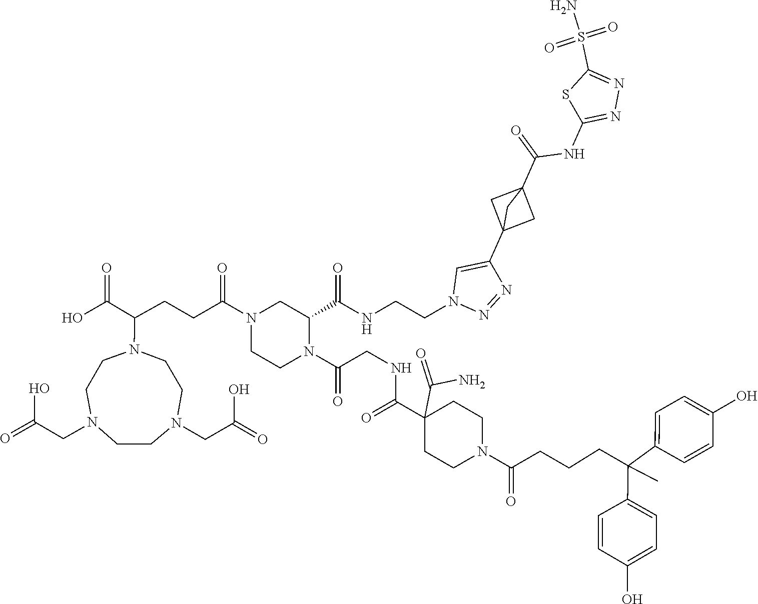 [SMILES: CC(CCCC(=O)N1CCC(C(N)=O)(C(=O)NCC(=O)N2CCN(C(=O)CCC(C(=O)O)N3CCN(CC(=O)O)CCN(CC(=O)O)CC3)C[C@@H]2C(=O)NCCn2cc(C34CC(C(=O)Nc5nnc(S(N)(=O)=O)s5)(C3)C4)nn2)CC1)(c1ccc(O)cc1)c1ccc(O)cc1] |
| 127.4 | 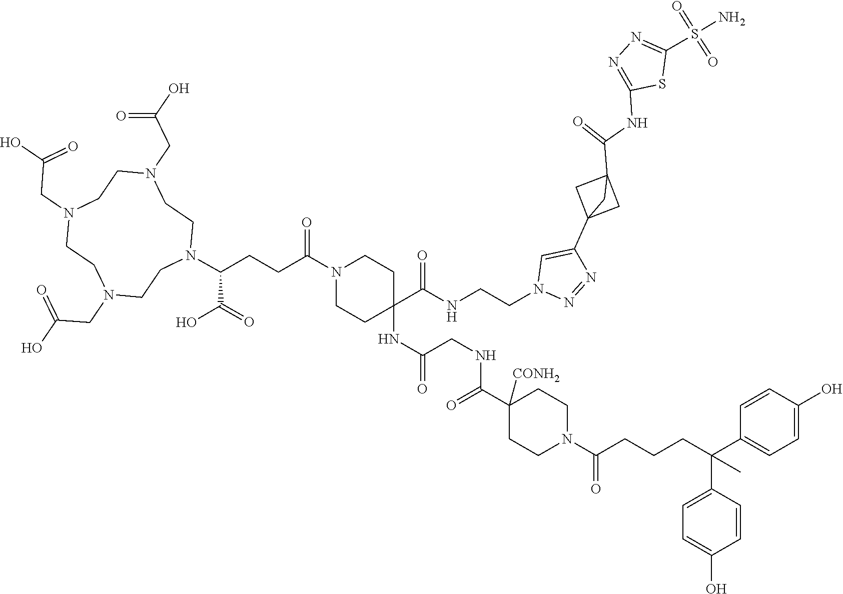 [SMILES: CC(CCCC(=O)N1CCC(C(N)=O)(C(=O)NCC(=O)NC2(C(=O)NCCn3cc(C45CC(C(=O)Nc6nnc(S(N)(=O)=O)s6)(C4)C5)nn3)CCN(C(=O)CC[C@H](C(=O)O)N3CCN(CC(=O)O)CCN(CC(=O)O)CCN(CC(=O)O)CC3)CC2)CC1)(c1ccc(O)cc1)c1ccc(O)cc1] |
| 127.5 | 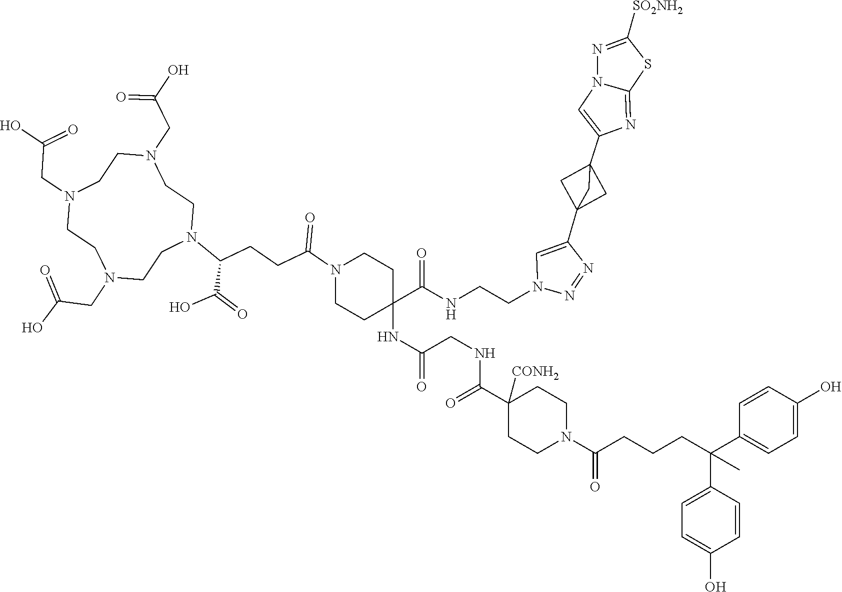 [SMILES: CC(CCCC(=O)N1CCC(C(N)=O)(C(=O)NCC(=O)NC2(C(=O)NCCn3cc(C45CC(c6cn7nc(S(N)(=O)=O)sc7n6)(C4)C5)nn3)CCN(C(=O)CC[C@H](C(=O)O)N3CCN(CC(=O)O)CCN(CC(=O)O)CCN(CC(=O)O)CC3)CC2)CC1)(c1ccc(O)cc1)c1ccc(O)cc1] |
| 127.6 | 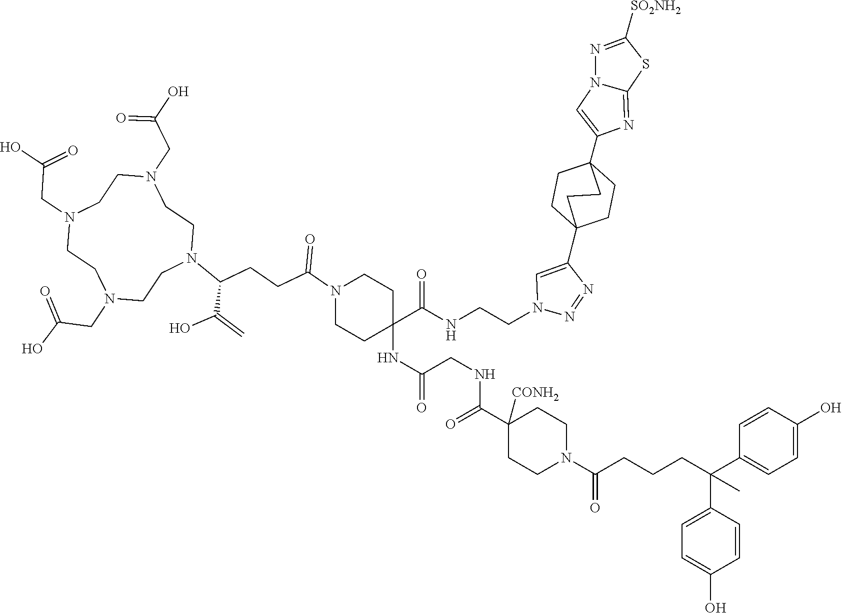 [SMILES: C=C(O)[C@@H](CCC(=O)N1CCC(NC(=O)CNC(=O)C2(C(N)=O)CCN(C(=O)CCCC(C)(c3ccc(O)cc3)c3ccc(O)cc3)CC2)(C(=O)NCCn2cc(C34CCC(c5cn6nc(S(N)(=O)=O)sc6n5)(CC3)CC4)nn2)CC1)N1CCN(CC(=O)O)CCN(CC(=O)O)CCN(CC(=O)O)CC1] |
| 127.7 | 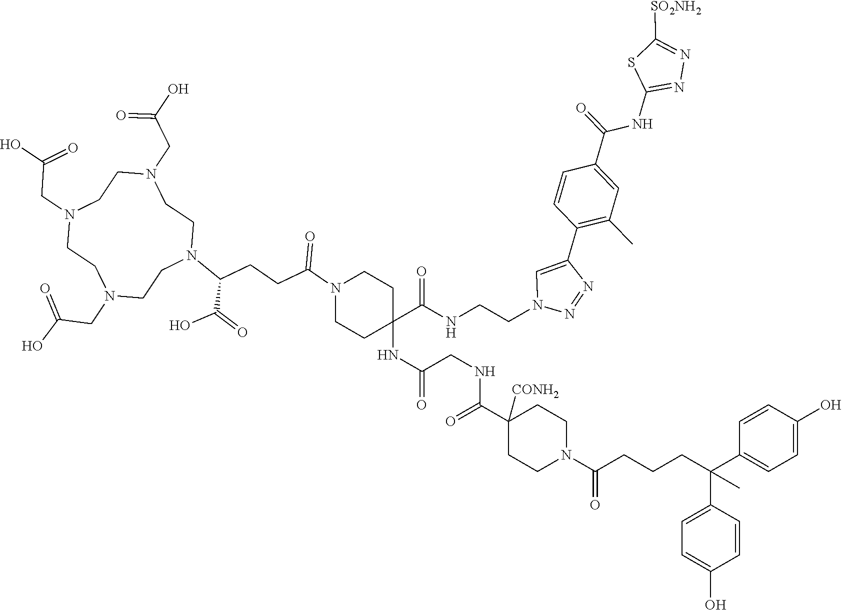 [SMILES: Cc1cc(C(=O)Nc2nnc(S(N)(=O)=O)s2)ccc1-c1cn(CCNC(=O)C2(NC(=O)CNC(=O)C3(C(N)=O)CCN(C(=O)CCCC(C)(c4ccc(O)cc4)c4ccc(O)cc4)CC3)CCN(C(=O)CC[C@H](C(=O)O)N3CCN(CC(=O)O)CCN(CC(=O)O)CCN(CC(=O)O)CC3)CC2)nn1] |
| 127.8 | 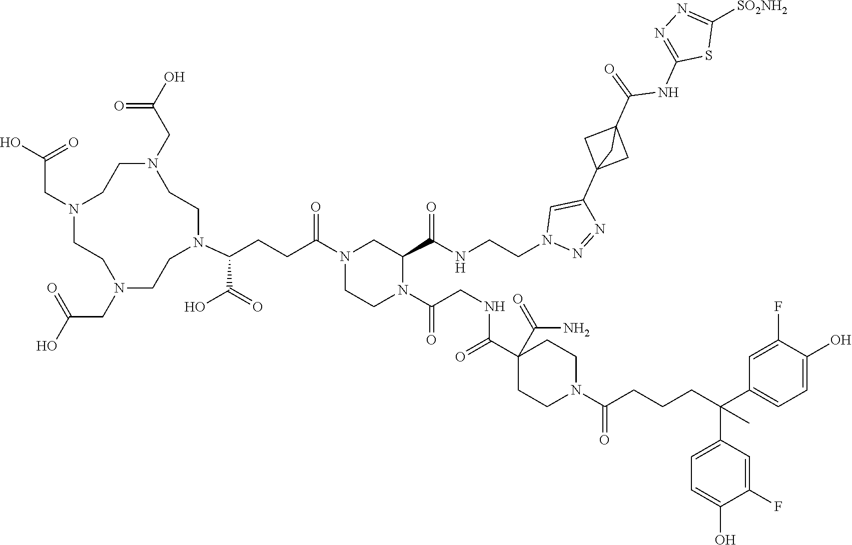 [SMILES: CC(CCCC(=O)N1CCC(C(N)=O)(C(=O)NCC(=O)N2CCN(C(=O)CC[C@H](C(=O)O)N3CCN(CC(=O)O)CCN(CC(=O)O)CCN(CC(=O)O)CC3)C[C@H]2C(=O)NCCn2cc(C34CC(C(=O)Nc5nnc(S(N)(=O)=O)s5)(C3)C4)nn2)CC1)(c1ccc(O)c(F)c1)c1ccc(O)c(F)c1] |
| 127.9 | 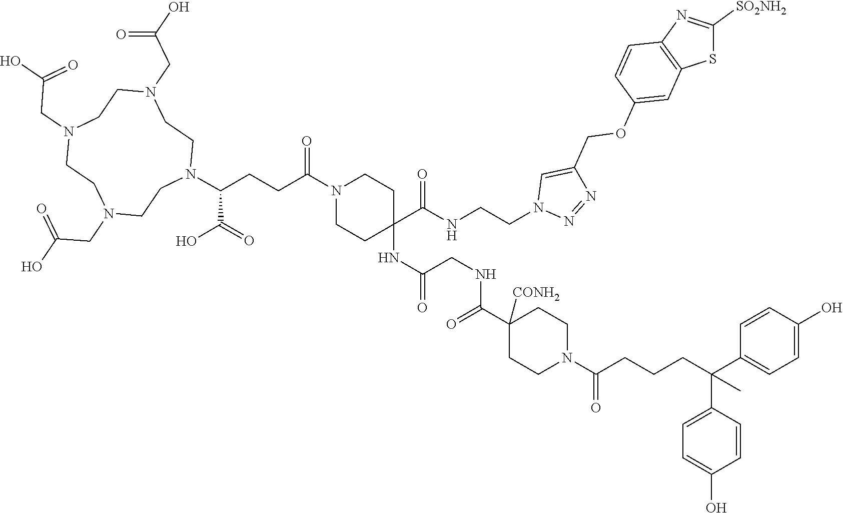 [SMILES: CC(CCCC(=O)N1CCC(C(N)=O)(C(=O)NCC(=O)NC2(C(=O)NCCn3cc(COc4ccc5nc(S(N)(=O)=O)sc5c4)nn3)CCN(C(=O)CC[C@H](C(=O)O)N3CCN(CC(=O)O)CCN(CC(=O)O)CCN(CC(=O)O)CC3)CC2)CC1)(c1ccc(O)cc1)c1ccc(O)cc1] |
| 128 | 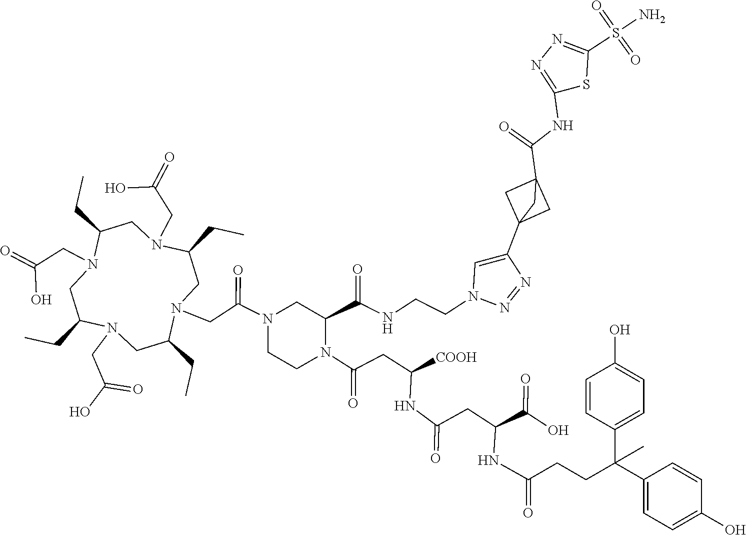 [SMILES: CC[C@H]1CN(CC(=O)O)[C@@H](CC)CN(CC(=O)N2CCN(C(=O)C[C@H](NC(=O)C[C@H](NC(=O)CCC(C)(c3ccc(O)cc3)c3ccc(O)cc3)C(=O)O)C(=O)O)[C@H](C(=O)NCCn3cc(C45CC(C(=O)Nc6nnc(S(N)(=O)=O)s6)(C4)C5)nn3)C2)[C@@H](CC)CN(CC(=O)O)[C@@H](CC)CN1CC(=O)O] |
| 128.1 | 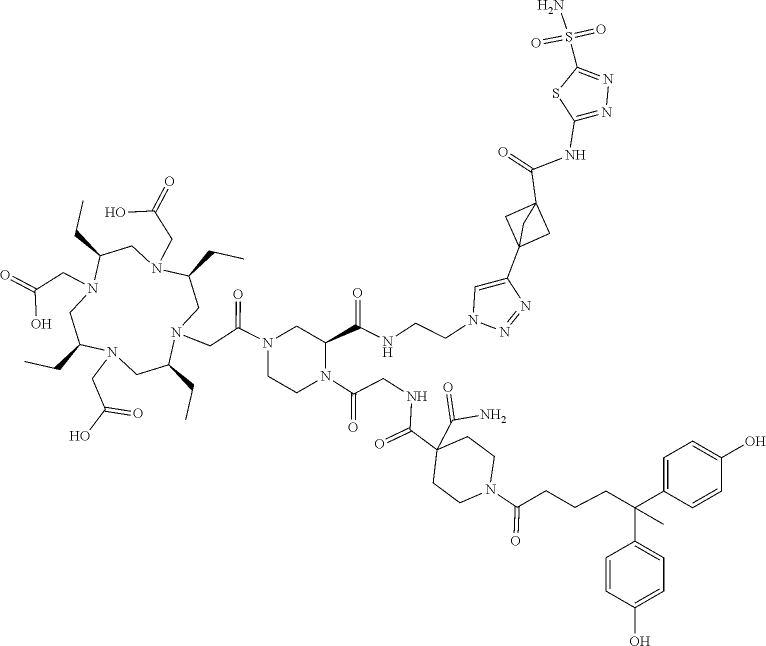 [SMILES: CC[C@H]1CN(CC(=O)O)[C@@H](CC)CN(CC(=O)N2CCN(C(=O)CNC(=O)C3(C(N)=O)CCN(C(=O)CCCC(C)(c4ccc(O)cc4)c4ccc(O)cc4)CC3)[C@H](C(=O)NCCn3cc(C45CC(C(=O)Nc6nnc(S(N)(=O)=O)s6)(C4)C5)nn3)C2)[C@@H](CC)CN(CC(=O)O)[C@@H](CC)CN1CC(=O)O] |
| 129 | 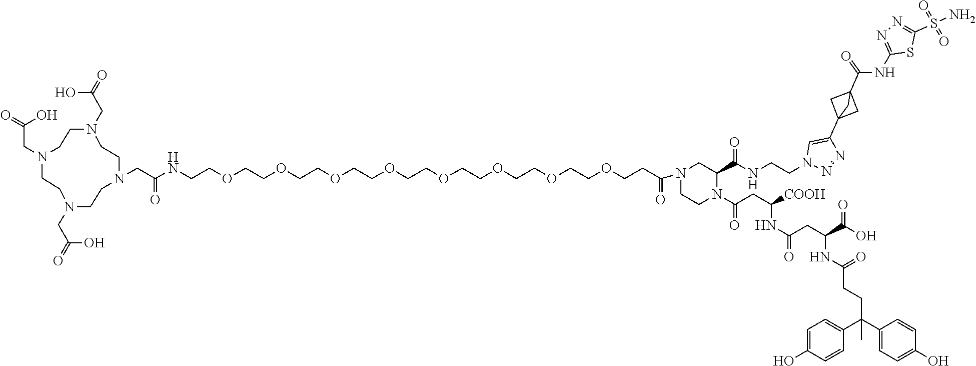 [SMILES: CC(CCC(=O)N[C@@H](CC(=O)N[C@@H](CC(=O)N1CCN(C(=O)CCOCCOCCOCCOCCOCCOCCOCCOCCNC(=O)CN2CCN(CC(=O)O)CCN(CC(=O)O)CCN(CC(=O)O)CC2)C[C@H]1C(=O)NCCn1cc(C23CC(C(=O)Nc4nnc(S(N)(=O)=O)s4)(C2)C3)nn1)C(=O)O)C(=O)O)(c1ccc(O)cc1)c1ccc(O)cc1] |
| 129.1 | 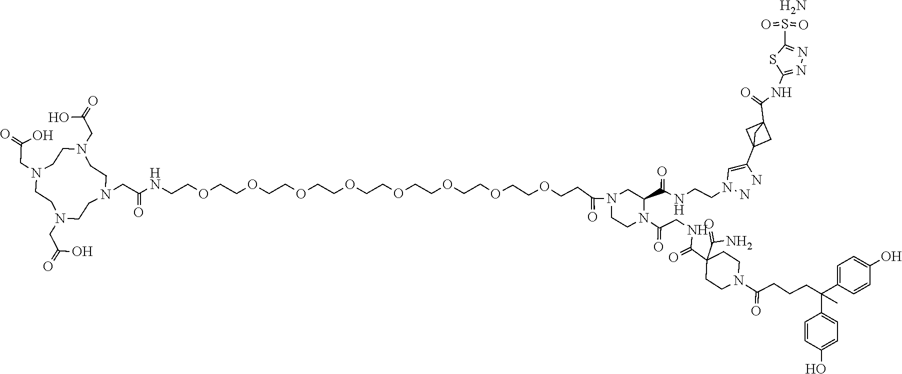 [SMILES: CC(CCCC(=O)N1CCC(C(N)=O)(C(=O)NCC(=O)N2CCN(C(=O)CCOCCOCCOCCOCCOCCOCCOCCOCCNC(=O)CN3CCN(CC(=O)O)CCN(CC(=O)O)CCN(CC(=O)O)CC3)C[C@H]2C(=O)NCCn2cc(C34CC(C(=O)Nc5nnc(S(N)(=O)=O)s5)(C3)C4)nn2)CC1)(c1ccc(O)cc1)c1ccc(O)cc1] |
| 129.2 | 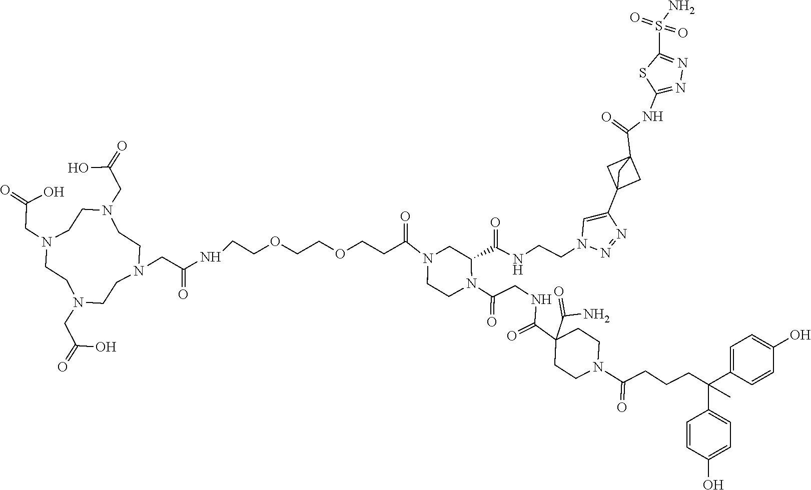 [SMILES: CC(CCCC(=O)N1CCC(C(N)=O)(C(=O)NCC(=O)N2CCN(C(=O)CCOCCOCCNC(=O)CN3CCN(CC(=O)O)CCN(CC(=O)O)CCN(CC(=O)O)CC3)C[C@@H]2C(=O)NCCn2cc(C34CC(C(=O)Nc5nnc(S(N)(=O)=O)s5)(C3)C4)nn2)CC1)(c1ccc(O)cc1)c1ccc(O)cc1] |
| 129.3 | 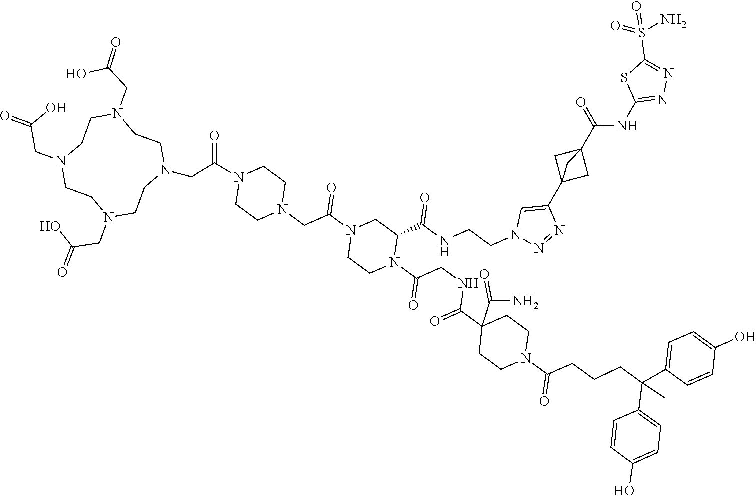 [SMILES: CC(CCCC(=O)N1CCC(C(N)=O)(C(=O)NCC(=O)N2CCN(C(=O)CN3CCN(C(=O)CN4CCN(CC(=O)O)CCN(CC(=O)O)CCN(CC(=O)O)CC4)CC3)C[C@@H]2C(=O)NCCn2cc(C34CC(C(=O)Nc5nnc(S(N)(=O)=O)s5)(C3)C4)nn2)CC1)(c1ccc(O)cc1)c1ccc(O)cc1] |
| 129.4 | 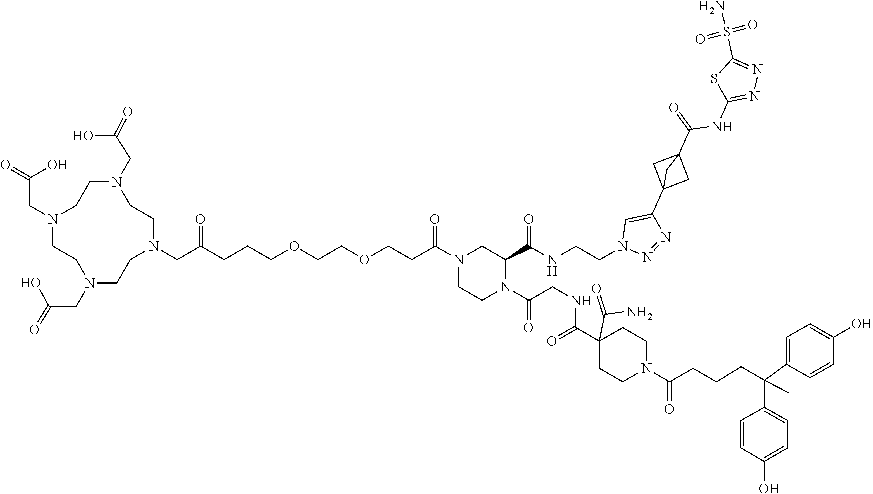 [SMILES: CC(CCCC(=O)N1CCC(C(N)=O)(C(=O)NCC(=O)N2CCN(C(=O)CCOCCOCCCC(=O)CN3CCN(CC(=O)O)CCN(CC(=O)O)CCN(CC(=O)O)CC3)C[C@H]2C(=O)NCCn2cc(C34CC(C(=O)Nc5nnc(S(N)(=O)=O)s5)(C3)C4)nn2)CC1)(c1ccc(O)cc1)c1ccc(O)cc1] |
| 129.5 | 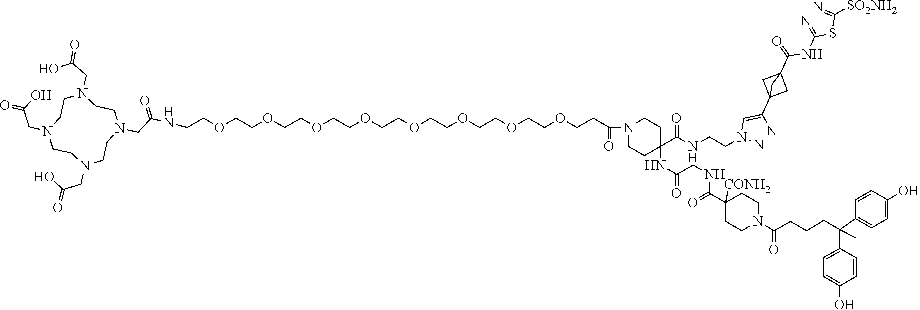 [SMILES: CC(CCCC(=O)N1CCC(C(N)=O)(C(=O)NCC(=O)NC2(C(=O)NCCn3cc(C45CC(C(=O)Nc6nnc(S(N)(=O)=O)s6)(C4)C5)nn3)CCN(C(=O)CCOCCOCCOCCOCCOCCOCCOCCOCCNC(=O)CN3CCN(CC(=O)O)CCN(CC(=O)O)CCN(CC(=O)O)CC3)CC2)CC1)(c1ccc(O)cc1)c1ccc(O)cc1] |
| 129.6 | 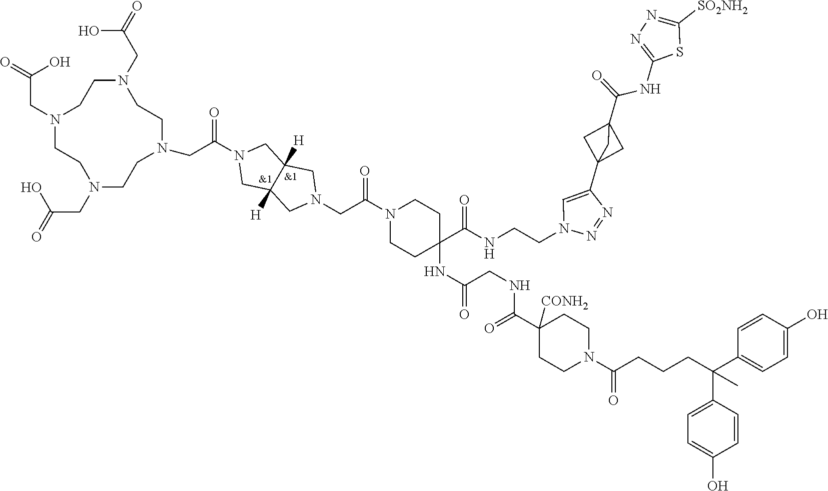 [SMILES: CC(CCCC(=O)N1CCC(C(N)=O)(C(=O)NCC(=O)NC2(C(=O)NCCn3cc(C45CC(C(=O)Nc6nnc(S(N)(=O)=O)s6)(C4)C5)nn3)CCN(C(=O)CN3C[C@@H]4CN(C(=O)CN5CCN(CC(=O)O)CCN(CC(=O)O)CCN(CC(=O)O)CC5)C[C@@H]4C3)CC2)CC1)(c1ccc(O)cc1)c1ccc(O)cc1] |
| 129.7 | 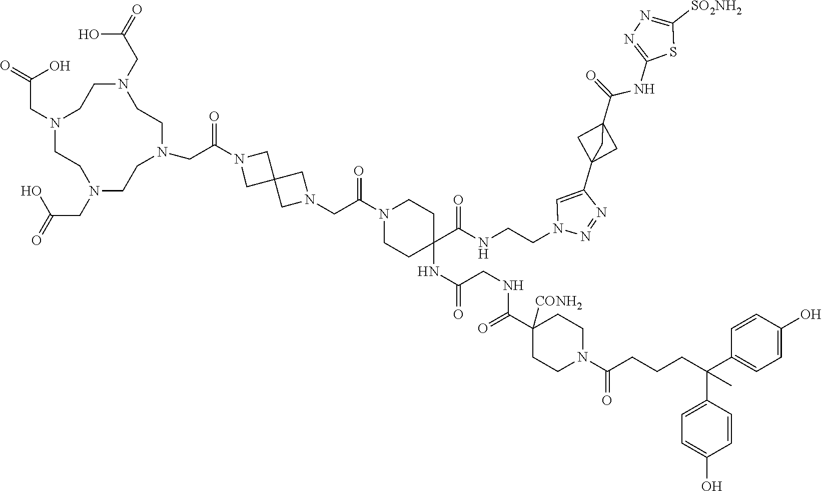 [SMILES: CC(CCCC(=O)N1CCC(C(N)=O)(C(=O)NCC(=O)NC2(C(=O)NCCn3cc(C45CC(C(=O)Nc6nnc(S(N)(=O)=O)s6)(C4)C5)nn3)CCN(C(=O)CN3CC4(C3)CN(C(=O)CN3CCN(CC(=O)O)CCN(CC(=O)O)CCN(CC(=O)O)CC3)C4)CC2)CC1)(c1ccc(O)cc1)c1ccc(O)cc1] |
| 130 | 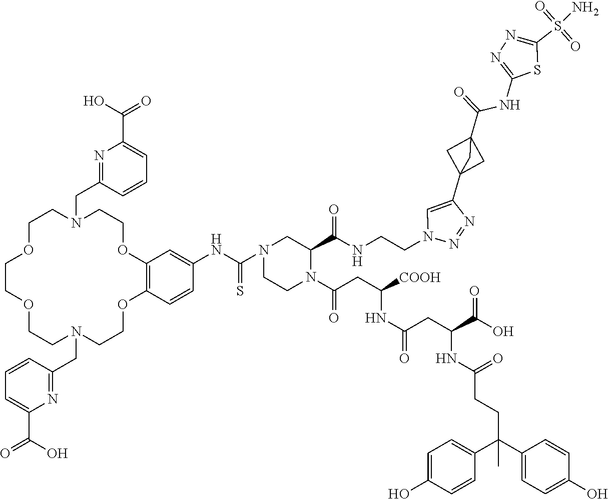 [SMILES: CC(CCC(=O)N[C@@H](CC(=O)N[C@@H](CC(=O)N1CCN(C(=S)Nc2ccc3c(c2)OCCN(Cc2cccc(C(=O)O)n2)CCOCCOCCN(Cc2cccc(C(=O)O)n2)CCO3)C[C@H]1C(=O)NCCn1cc(C23CC(C(=O)Nc4nnc(S(N)(=O)=O)s4)(C2)C3)nn1)C(=O)O)C(=O)O)(c1ccc(O)cc1)c1ccc(O)cc1] |
| 131 | 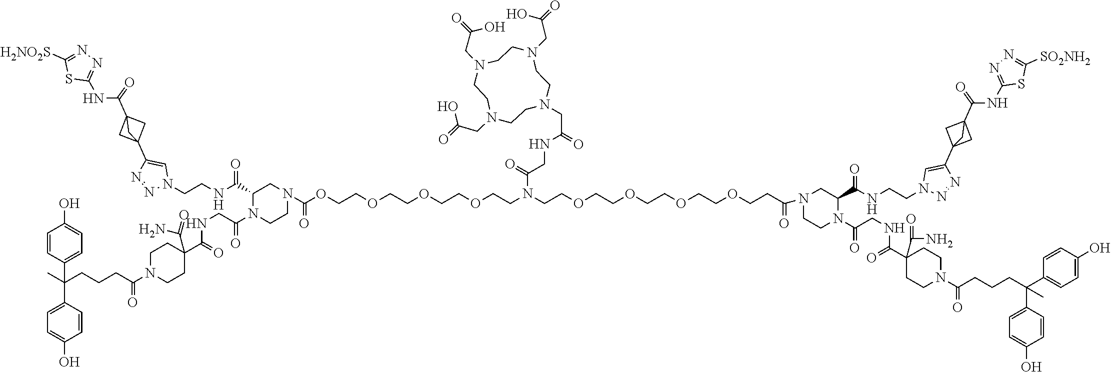 [SMILES: CC(CCCC(=O)N1CCC(C(N)=O)(C(=O)NCC(=O)N2CCN(C(=O)CCOCCOCCOCCOCCN(CCOCCOCCOCCOC(=O)N3CCN(C(=O)CNC(=O)C4(C(N)=O)CCN(C(=O)CCCC(C)(c5ccc(O)cc5)c5ccc(O)cc5)CC4)[C@H](C(=O)NCCn4cc(C56CC(C(=O)Nc7nnc(S(N)(=O)=O)s7)(C5)C6)nn4)C3)C(=O)CNC(=O)CN3CCN(CC(=O)O)CCN(CC(=O)O)CCN(CC(=O)O)CC3)C[C@H]2C(=O)NCCn2cc(C34CC(C(=O)Nc5nnc(S(N)(=O)=O)s5)(C3)C4)nn2)CC1)(c1ccc(O)cc1)c1ccc(O)cc1] |
| 131.1 | 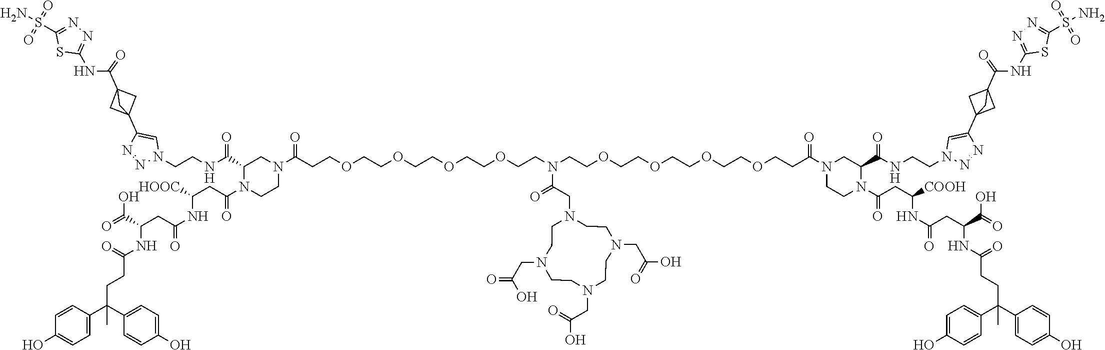 [SMILES: CC(CCC(=O)N[C@@H](CC(=O)N[C@@H](CC(=O)N1CCN(C(=O)CCOCCOCCOCCOCCN(CCOCCOCCOCCOCCC(=O)N2CCN(C(=O)C[C@H](NC(=O)C[C@H](NC(=O)CCC(C)(c3ccc(O)cc3)c3ccc(O)cc3)C(=O)O)C(=O)O)[C@H](C(=O)NCCn3cc(C45CC(C(=O)Nc6nnc(S(N)(=O)=O)s6)(C4)C5)nn3)C2)C(=O)CN2CCN(CC(=O)O)CCN(CC(=O)O)CCN(CC(=O)O)CC2)C[C@H]1C(=O)NCCn1cc(C23CC(C(=O)Nc4nnc(S(N)(=O)=O)s4)(C2)C3)nn1)C(=O)O)C(=O)O)(c1ccc(O)cc1)c1ccc(O)cc1] |
| 131.2 | 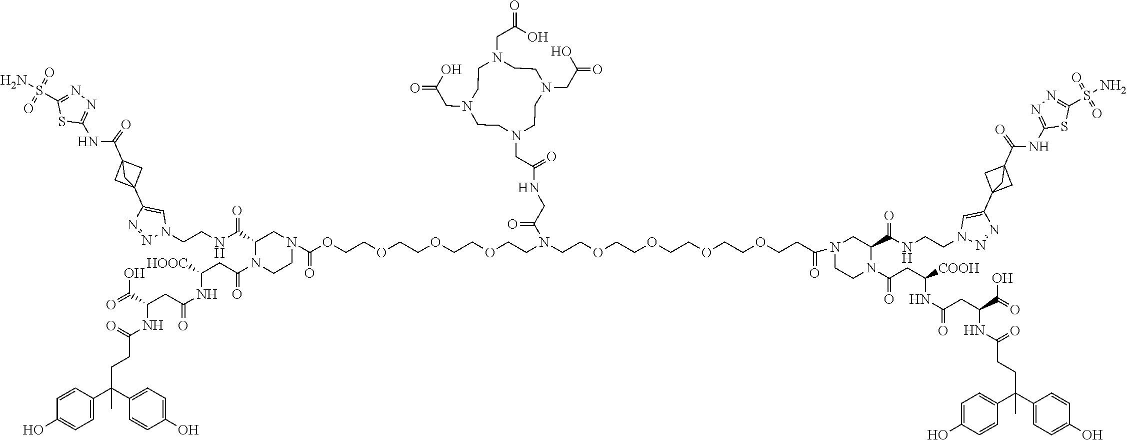 [SMILES: CC(CCC(=O)N[C@@H](CC(=O)N[C@@H](CC(=O)N1CCN(C(=O)CCOCCOCCOCCOCCN(CCOCCOCCOCCOC(=O)N2CCN(C(=O)C[C@H](NC(=O)C[C@H](NC(=O)CCC(C)(c3ccc(O)cc3)c3ccc(O)cc3)C(=O)O)C(=O)O)[C@H](C(=O)NCCn3cc(C45CC(C(=O)Nc6nnc(S(N)(=O)=O)s6)(C4)C5)nn3)C2)C(=O)CNC(=O)CN2CCN(CC(=O)O)CCN(CC(=O)O)CCN(CC(=O)O)CC2)C[C@H]1C(=O)NCCn1cc(C23CC(C(=O)Nc4nnc(S(N)(=O)=O)s4)(C2)C3)nn1)C(=O)O)C(=O)O)(c1ccc(O)cc1)c1ccc(O)cc1] |
| 131.3 | 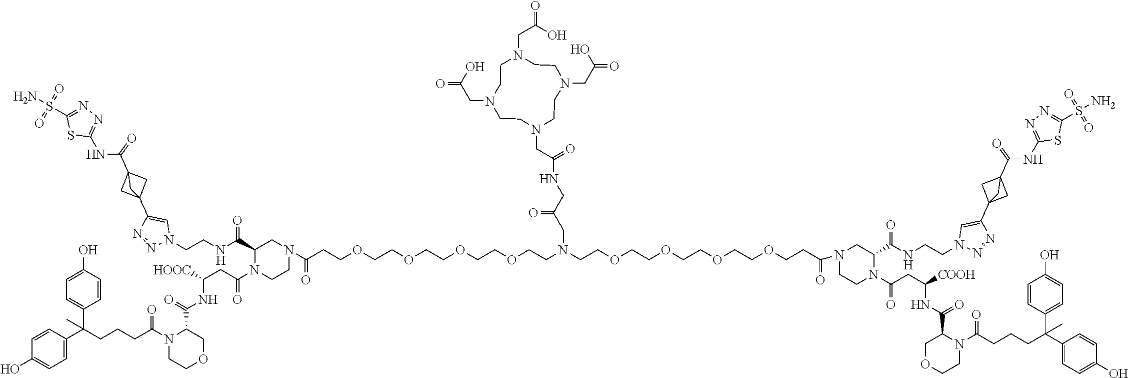 [SMILES: CC(CCCC(=O)N1CCOC[C@H]1C(=O)N[C@@H](CC(=O)N1CCN(C(=O)CCOCCOCCOCCOCCN(CCOCCOCCOCCOCCC(=O)N2CCN(C(=O)C[C@H](NC(=O)[C@@H]3COCCN3C(=O)CCCC(C)(c3ccc(O)cc3)c3ccc(O)cc3)C(=O)O)[C@@H](C(=O)NCCn3cc(C45CC(C(=O)Nc6nnc(S(N)(=O)=O)s6)(C4)C5)nn3)C2)CC(=O)CNC(=O)CN2CCN(CC(=O)O)CCN(CC(=O)O)CCN(CC(=O)O)CC2)C[C@@H]1C(=O)NCCn1cc(C23CC(C(=O)Nc4nnc(S(N)(=O)=O)s4)(C2)C3)nn1)C(=O)O)(c1ccc(O)cc1)c1ccc(O)cc1] |
| 131.4 | 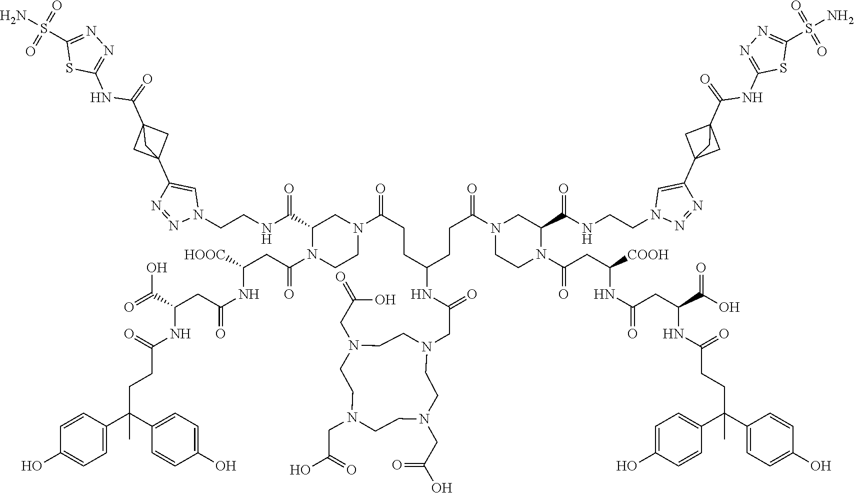 [SMILES: CC(CCC(=O)N[C@@H](CC(=O)N[C@@H](CC(=O)N1CCN(C(=O)CCC(CCC(=O)N2CCN(C(=O)C[C@H](NC(=O)C[C@H](NC(=O)CCC(C)(c3ccc(O)cc3)c3ccc(O)cc3)C(=O)O)C(=O)O)[C@H](C(=O)NCCn3cc(C45CC(C(=O)Nc6nnc(S(N)(=O)=O)s6)(C4)C5)nn3)C2)NC(=O)CN2CCN(CC(=O)O)CCN(CC(=O)O)CCN(CC(=O)O)CC2)C[C@H]1C(=O)NCCn1cc(C23CC(C(=O)Nc4nnc(S(N)(=O)=O)s4)(C2)C3)nn1)C(=O)O)C(=O)O)(c1ccc(O)cc1)c1ccc(O)cc1] |
| 131.5 | 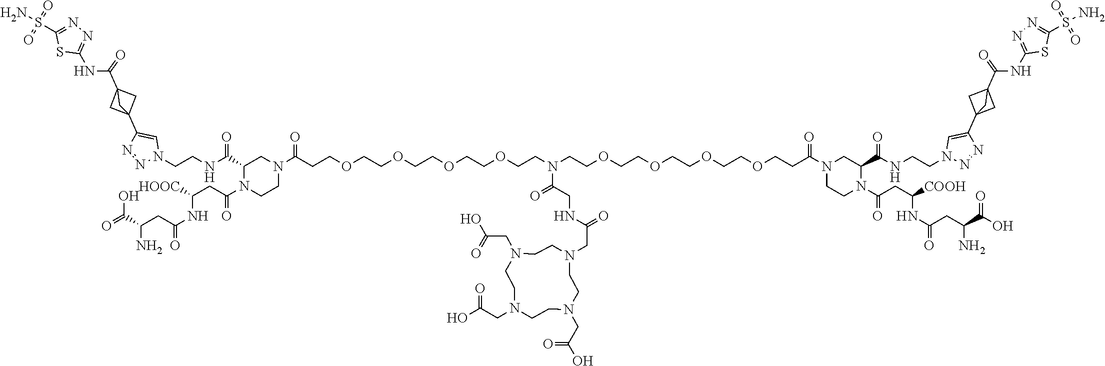 [SMILES: N[C@@H](CC(=O)N[C@@H](CC(=O)N1CCN(C(=O)CCOCCOCCOCCOCCN(CCOCCOCCOCCOCCC(=O)N2CCN(C(=O)C[C@H](NC(=O)C[C@H](N)C(=O)O)C(=O)O)[C@H](C(=O)NCCn3cc(C45CC(C(=O)Nc6nnc(S(N)(=O)=O)s6)(C4)C5)nn3)C2)C(=O)CNC(=O)CN2CCN(CC(=O)O)CCN(CC(=O)O)CCN(CC(=O)O)CC2)C[C@H]1C(=O)NCCn1cc(C23CC(C(=O)Nc4nnc(S(N)(=O)=O)s4)(C2)C3)nn1)C(=O)O)C(=O)O] |
| 131.6 | 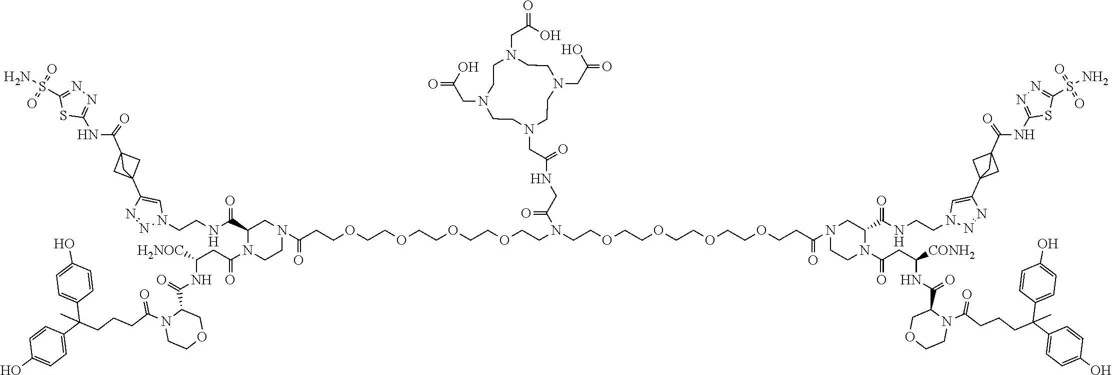 [SMILES: CC(CCCC(=O)N1CCOC[C@H]1C(=O)N[C@@H](CC(=O)N1CCN(C(=O)CCOCCOCCOCCOCCN(CCOCCOCCOCCOCCC(=O)N2CCN(C(=O)C[C@H](NC(=O)[C@@H]3COCCN3C(=O)CCCC(C)(c3ccc(O)cc3)c3ccc(O)cc3)C(N)=O)[C@@H](C(=O)NCCn3cc(C45CC(C(=O)Nc6nnc(S(N)(=O)=O)s6)(C4)C5)nn3)C2)C(=O)CNC(=O)CN2CCN(CC(=O)O)CCN(CC(=O)O)CCN(CC(=O)O)CC2)C[C@@H]1C(=O)NCCn1cc(C23CC(C(=O)Nc4nnc(S(N)(=O)=O)s4)(C2)C3)nn1)C(N)=O)(c1ccc(O)cc1)c1ccc(O)cc1] |
| 131.7 | 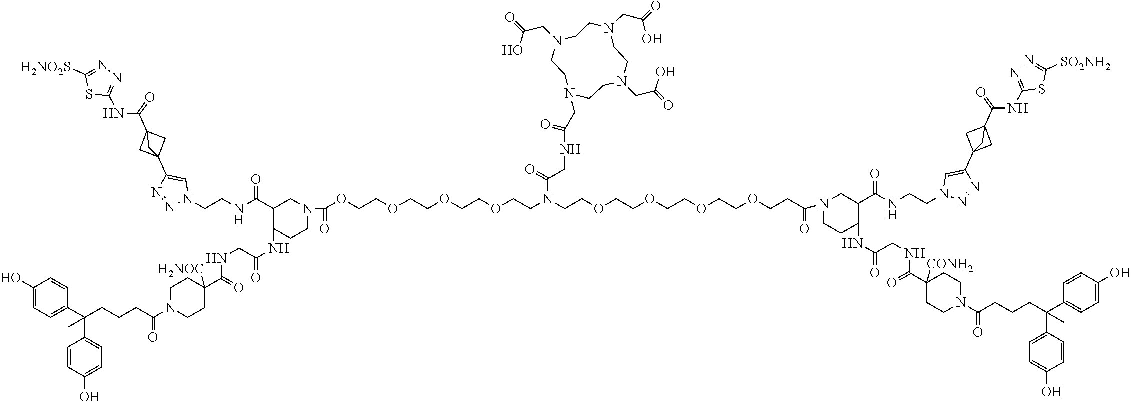 [SMILES: CC(CCCC(=O)N1CCC(C(N)=O)(C(=O)NCC(=O)NC2CCN(C(=O)CCOCCOCCOCCOCCN(CCOCCOCCOCCOC(=O)N3CCC(NC(=O)CNC(=O)C4(C(N)=O)CCN(C(=O)CCCC(C)(c5ccc(O)cc5)c5ccc(O)cc5)CC4)C(C(=O)NCCn4cc(C56CC(C(=O)Nc7nnc(S(N)(=O)=O)s7)(C5)C6)nn4)C3)C(=O)CNC(=O)CN3CCN(CC(=O)O)CCN(CC(=O)O)CCN(CC(=O)O)CC3)CC2C(=O)NCCn2cc(C34CC(C(=O)Nc5nnc(S(N)(=O)=O)s5)(C3)C4)nn2)CC1)(c1ccc(O)cc1)c1ccc(O)cc1] |
| 132 | 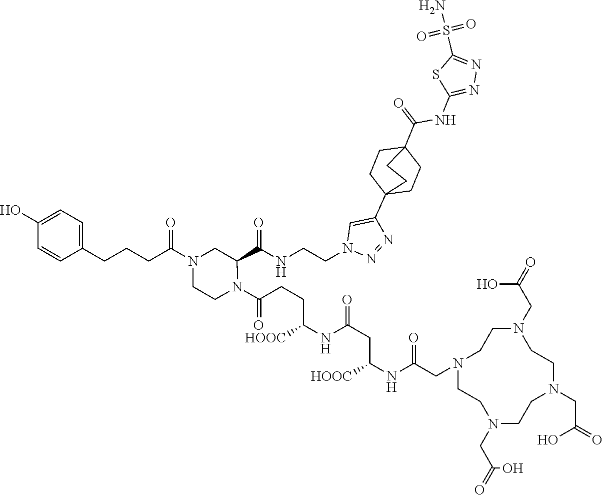 [SMILES: NS(=O)(=O)c1nnc(NC(=O)C23CCC(c4cn(CCNC(=O)[C@@H]5CN(C(=O)CCCc6ccc(O)cc6)CCN5C(=O)CC[C@H](NC(=O)C[C@H](NC(=O)CN5CCN(CC(=O)O)CCN(CC(=O)O)CCN(CC(=O)O)CC5)C(=O)O)C(=O)O)nn4)(CC2)CC3)s1] |
| 133 | 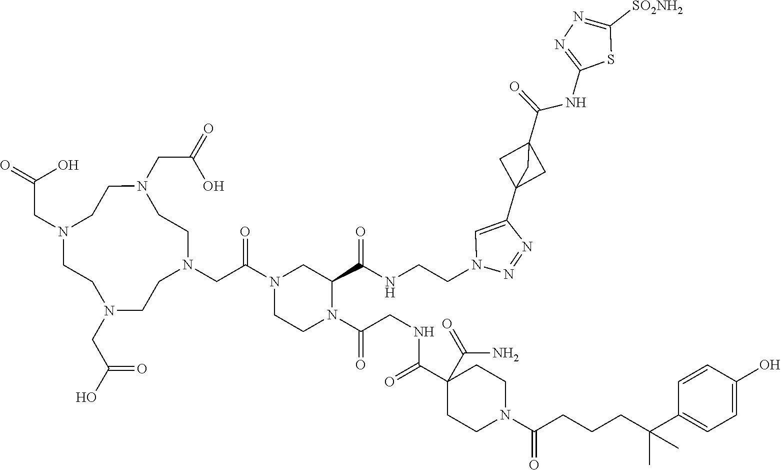 [SMILES: CC(C)(CCCC(=O)N1CCC(C(N)=O)(C(=O)NCC(=O)N2CCN(C(=O)CN3CCN(CC(=O)O)CCN(CC(=O)O)CCN(CC(=O)O)CC3)C[C@H]2C(=O)NCCn2cc(C34CC(C(=O)Nc5nnc(S(N)(=O)=O)s5)(C3)C4)nn2)CC1)c1ccc(O)cc1] |
| 134 | 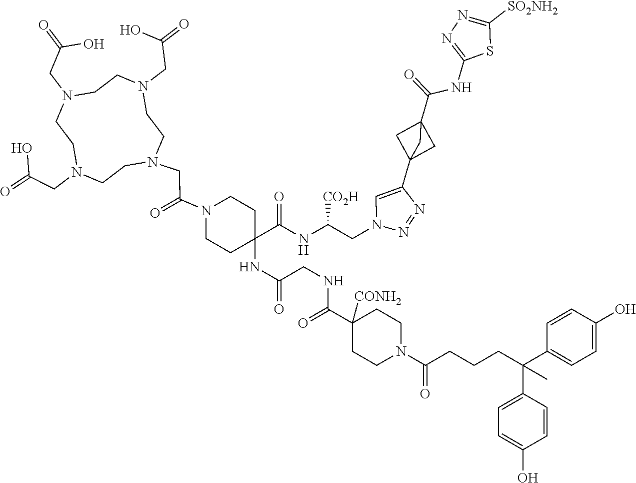 [SMILES: CC(CCCC(=O)N1CCC(C(N)=O)(C(=O)NCC(=O)NC2(C(=O)N[C@@H](Cn3cc(C45CC(C(=O)Nc6nnc(S(N)(=O)=O)s6)(C4)C5)nn3)C(=O)O)CCN(C(=O)CN3CCN(CC(=O)O)CCN(CC(=O)O)CCN(CC(=O)O)CC3)CC2)CC1)(c1ccc(O)cc1)c1ccc(O)cc1] |
| 135 | 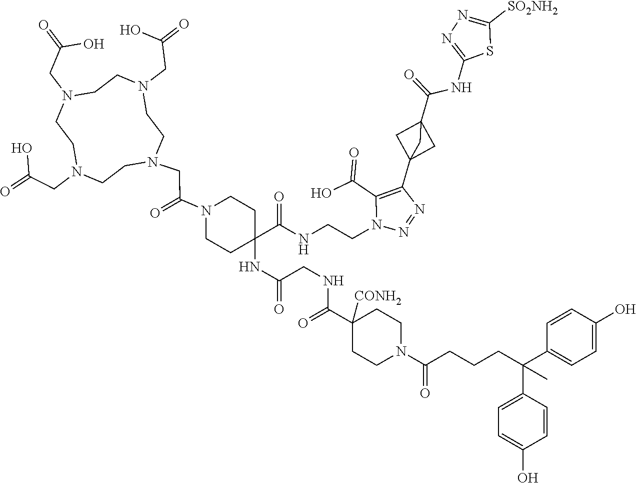 [SMILES: CC(CCCC(=O)N1CCC(C(N)=O)(C(=O)NCC(=O)NC2(C(=O)NCCn3nnc(C45CC(C(=O)Nc6nnc(S(N)(=O)=O)s6)(C4)C5)c3C(=O)O)CCN(C(=O)CN3CCN(CC(=O)O)CCN(CC(=O)O)CCN(CC(=O)O)CC3)CC2)CC1)(c1ccc(O)cc1)c1ccc(O)cc1] |
| 136 | 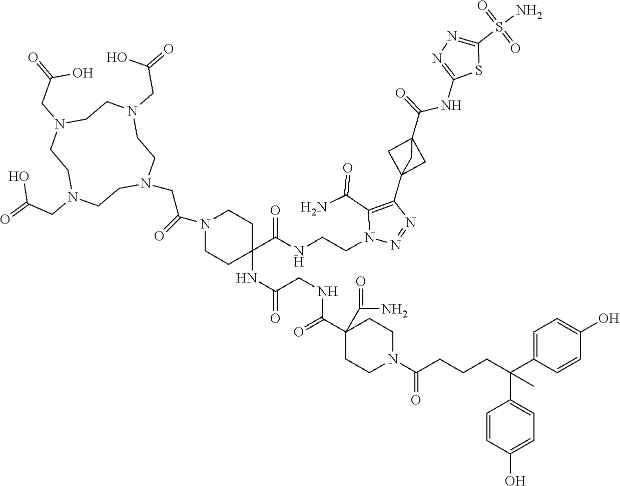 [SMILES: CC(CCCC(=O)N1CCC(C(N)=O)(C(=O)NCC(=O)NC2(C(=O)NCCn3nnc(C45CC(C(=O)Nc6nnc(S(N)(=O)=O)s6)(C4)C5)c3C(N)=O)CCN(C(=O)CN3CCN(CC(=O)O)CCN(CC(=O)O)CCN(CC(=O)O)CC3)CC2)CC1)(c1ccc(O)cc1)c1ccc(O)cc1] |

In some embodiments the conjugate disclosed herein, or a pharmaceutically acceptable salt or stereoisomer thereof, is one of the compounds in Table 2.

TABLE 2
Structures

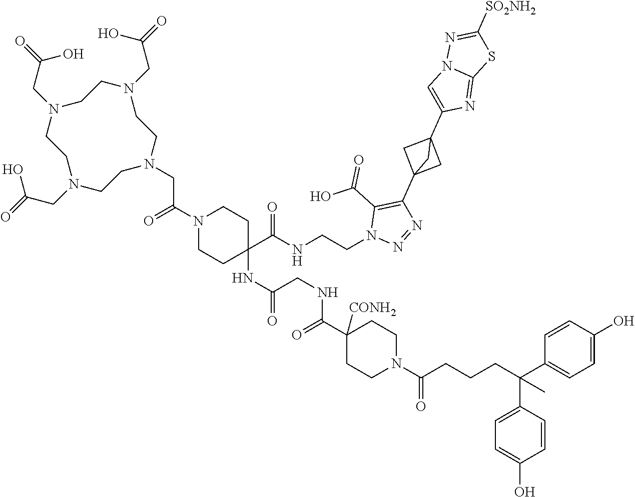 [SMILES: CC(CCCC(=O)N1CCC(C(N)=O)(C(=O)NCC(=O)NC2(C(=O)NCCn3nnc(C45CC(c6cn7nc(S(N)(=O)=O)sc7n6)(C4)C5)c3C(=O)O)CCN(C(=O)CN3CCN(CC(=O)O)CCN(CC(=O)O)CCN(CC(=O)O)CC3)CC2)CC1)(c1ccc(O)cc1)c1ccc(O)cc1]

### Further Forms of Compounds Disclosed Herein

### Isomers/Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

### Isotopically Enriched Compounds

Unless otherwise stated, compounds described herein may exhibit their natural isotopic abundance, or one or more of the atoms may be artificially enriched in a particular isotope having the same atomic number, but an atomic mass or mass number different from the atomic mass or mass number predominantly found in nature. All isotopic variations of the compounds of the present disclosure, whether radioactive or not, are encompassed within the scope of the present disclosure. For example, hydrogen has three naturally occurring isotopes, denoted ¹H (protium), ²H (deuterium), and ³H (tritium). Protium is the most abundant isotope of hydrogen in nature. Enriching for deuterium may afford some therapeutic advantages, such as increased in vivo half-life and/or exposure, or may provide a compound useful for investigating in vivo routes of drug elimination and metabolism.

For example, the compounds described herein may be artificially enriched in one or more particular isotopes. In some embodiments, the compounds described herein may be artificially enriched in one or more isotopes that are not predominantly found in nature. In some embodiments, the compounds described herein may be artificially enriched in one or more isotopes selected from deuterium (²H), tritium (³H), iodine-125 (¹²⁵I) or carbon-14 (¹⁴C). In some embodiments, the compounds described herein are artificially enriched in one or more isotopes selected from ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹³¹I, and ¹²⁵I. In some embodiments, the abundance of the enriched isotopes is independently at least 1%, at least 10%, at least 20%, at least 30%, at least 40%, at least 50%, at least 60%, at least 70%, at least 80%, at least 90%, or 100% by molar.

In some embodiments, the compound is deuterated in at least one position. In some embodiments, the compounds disclosed herein have some or all of the ¹H atoms replaced with ²H atoms.

The methods of synthesis for deuterium-containing compounds are known in the art and include, by way of non-limiting example only, the procedure described in U.S. Pat. Nos. 5,846,514 and 6,334,997, and the following synthetic methods. For example, deuterium substituted compounds may be synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)]2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein or stereoisomer thereof, or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undecanoate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. In some embodiments, other acids, such as oxalic, while not in themselves pharmaceutically acceptable, are employed in the preparation of salts useful as intermediates in obtaining the compounds disclosed herein or stereoisomer thereof and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they contain. In some embodiments, water or oil-soluble or dispersible products are obtained by such quaternization.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Radiopharmaceutical Conjugates

Disclosed herein are radiopharmaceutical conjugate comprising a conjugate disclosed herein bound to a radionuclide. Generally, the type of radionuclide used in a therapeutic radiopharmaceutical can be tailored to the specific type of cancer, the type of targeting moiety (e.g., binding peptide), etc. Radionuclides that undergo α-decay produce particles composed of two neutrons and two protons, and radionuclides that undergo β-decay emit energetic electrons from their nuclei. Some radionuclides can also undergo electron capture and emit Auger electrons. In some embodiments, the conjugate comprises an alpha particle-emitting radionuclide. Alpha radiation can cause direct, irreparable double-strand DNA breaks compared with gamma and beta radiation, which can cause single-stranded breaks via indirect DNA damage. The range of these particles in tissue and the half-life of the radionuclide can also be considered in designing the radiopharmaceutical conjugate. Tables 3 below illustrates some properties of exemplary radionuclides.

TABLE 3
Exemplary radionuclides

| | | | Exemplary methods of binding |
|---|---|---|---|
| | | | the radionuclide to a |
| | | Exemplary | radiopharmaceutical conjugate |
| | | application-- | (e.g., through a Chelator |
| | | Therapeutics (Tx) | or bound to the conjugate |
| Radionuclide | Emission/Decay | or Diagnostics (Dx) | through a Covalent bond) |
| Ac-225 | Alpha | Tx | Chelator |
| As-70 | Positron | Dx | Covalent |
| As-71 | Positron | Dx | Covalent |
| As-72 | Positron | Dx | Covalent |
| As-73 | Electron Capture | Dx | Covalent |
| As-74 | Positron | Dx | Covalent |
| As-76 | Beta | Tx | Covalent |
| As-77 | Beta | Tx | Covalent |
| At-211 | Alpha | Tx | Covalent |
| Bi-209 | Alpha | Tx | Chelator |
| Bi-212 | Alpha | Tx | Chelator |
| Bi-213 | Alpha | Tx | Chelator |
| Br-76 | Positron | Dx | Covalent |
| Ce-134 | Electron Capture | Dx | Chelator |
| Cu-61 | Positron | Dx | Chelator |
| Cu-62 | Positron | Dx | Chelator |
| Cu-64 | Positron | Dx | Chelator |
| Cu-67 | Beta | Tx | Chelator |
| Fr-223 | Alpha | Tx | Chelator |
| Ga-68 | Positron | Dx | Chelator |
| Gd-148 | Alpha | Tx | Chelator |
| Ho-166 | Beta/gamma | Tx/Dx | Chelator |
| I-123 | Electron Capture | Dx | Covalent |
| I-124 | Positron | Dx | Covalent |
| I-125 | Electron Capture | Dx | Covalent |
| I-131 | Beta | Tx | Covalent |
| In-111 | Beta/Electron Capture | Tx/Dx | Chelator |
| Lu-177 | Beta | Tx | Chelator |
| Pb-212 | Alpha | Tx | Chelator |
| Pm-153 | Beta | Tx | Chelator |
| Po-213 | Alpha | Tx | Chelator |
| Ra-223 | Alpha | Tx | Chelator |
| Ra-224 | Alpha | Tx | Chelator |
| Rh-105 | Beta | Tx | Chelator |
| Sm-153 | Beta | Tx | Chelator |
| Tb-149 | Alpha | Tx | Chelator |
| Tb-152 | Positron | Dx | Chelator |
| Tb-161 | Beta | Tx/Dx | Chelator |
| Tc-99m | Gamma/Isomeric Transition | Dx | Chelator |
| Th-227 | Alpha | Tx | Chelator |
| Th-229 | Alpha | Tx | Chelator |
| Tm-167 | Beta/Electron Capture | Tx | Chelator |
| Y-90 | Beta | Tx | Chelator |
| Yb-175 | Beta | Tx | Chelator |
| Zr-89 | Positron | Dx | Chelator |

In some embodiments, the radiopharmaceutical conjugate described herein comprises a radionuclide selected from Table 3. In some embodiments, disclosed herein are conjugates comprising a compound of Table 1, or a salt or stereoisomer thereof, complexed with a radionuclide selected from Table 3. In some embodiments, disclosed herein are compositions comprising a compound of Table 1, or a salt or stereoisomer thereof, complexed with a radionuclide selected from Table 3. In some embodiments, disclosed herein are conjugates comprising a compound of Table 2, or a salt or stereoisomer thereof, complexed with a radionuclide selected from Table 3. In some embodiments, disclosed herein are compositions comprising a compound of Table 2, or a salt or stereoisomer thereof, complexed with a radionuclide selected from Table 3.

In some embodiments, the radiopharmaceutical conjugate described herein comprises one or more independent radionuclides. In some embodiments, the radiopharmaceutical conjugate comprises two radionuclides. In some embodiments, each of the one or more radionuclides is bound to the metal chelator of the radiopharmaceutical conjugate. In some embodiments, two radionuclides of the radiopharmaceutical conjugate are bound to the same metal chelator. In some embodiments, two radionuclides of the radiopharmaceutical conjugate are bound to two independent metal chelators. In some embodiments, each of the one or more radionuclides is an alpha particle-emitting radionuclide.

In some embodiments, the radiopharmaceutical conjugate described herein comprises an alpha particle-emitting radionuclide. In some embodiments, the radiopharmaceutical conjugate comprises an alpha-particle emitting radionuclide bound to the metal chelator. In some embodiments, the alpha particle-emitting radionuclide is actinium-225 (²²⁵Ac), radium-223 (²²³Ra), radium-224 (²²⁴Ra), bismuth-209 (²⁰⁹Bi), bismuth-213 (²¹³Bi), gadolinium-148 (¹⁴⁸Gd), terbium-149 (¹⁴⁹Tb), polonium-213 (²¹³Po), francium-223 (²²³Fr), thorium-227 (²²⁷Th), thorium-229 (²²⁹Th), or lead-212 (²¹²Pb). In some embodiments, the alpha particle-emitting radionuclide is selected from ²²⁵Ac, ²²³Ra, ²⁰⁹Bi, ²¹³Bi, ¹⁴⁸Gd, ¹⁴⁹Tb, ²¹³Po, ²²³Fr, ²²⁷Th, ²²⁹Th, and ²¹²Pb. In some embodiments, the alpha particle-emitting radionuclide is ²²⁵Ac. In some embodiments, the alpha particle-emitting radionuclide is ²¹³Bi. In some embodiments, the alpha particle-emitting radionuclide is ²¹²Bi. In some embodiments, the alpha particle-emitting radionuclide is ²¹²Pb. In some embodiments, the alpha particle-emitting radionuclide is ²²⁴Ra. In some embodiments, the alpha particle-emitting radionuclide is ²²³Ra. In some embodiments, the alpha particle-emitting radionuclide is ²²⁷Th. In some embodiments, the alpha particle-emitting radionuclide is ¹⁴⁹Tb.

In some embodiments, the radiopharmaceutical conjugate described herein comprises a radionuclide selected from ⁶²Cu, ⁶⁴Cu, ⁶⁷Cu, ⁹⁰Y, ¹⁰⁹Pd, ¹¹¹Ag, ¹³⁴Ce, ¹⁴⁹Pm, ¹⁵³Sm, ¹⁶⁶Ho, ^{99m}Tc, ⁶⁷Ga, ⁶⁸Ga, ¹¹¹In, ⁹⁰Y, ¹⁷⁷Lu, ¹⁸⁶Re, ¹⁸⁸Re, ¹⁹⁷Au, ¹⁹⁸Au, ¹⁹⁹Au, ¹⁰⁵Rh, ¹⁶⁵Ho, ¹⁶¹Tb, ¹⁴⁹Pm, ¹⁵³Pm, ⁴⁴Sc, ⁴⁷Sc, ²¹³Po, ²¹²Pb, ²⁰⁹Bi, ²¹²Bi, ²¹³Bi, ²²⁵Ac, ^{117m}Sn, ⁶⁷Ga, ¹⁴⁹Tb, ¹⁵²Tb, ¹⁶⁷Tm, ¹⁷⁵Yb, ²²³R^{a}, ²²³Fr, ²²⁷Th, ²²⁹Th, ²⁰¹Tl, ¹⁴⁸Gd, ¹⁶⁰Gd, ¹⁴⁸Nd, ⁸⁹Sr, and ⁸⁹Zr. In some embodiments, the radionuclide is selected from ⁶²Cu, ⁶⁴Cu, ⁶⁷Cu, ⁶⁸Ga, ⁸⁹Zr, ⁹⁰Y, ^{99m}Tc, ¹⁰⁵Rh, ¹¹¹In, ¹³⁴Ce, ¹⁴⁸Gd, ¹⁴⁹Tb, ¹⁵²Tb, ¹⁵³Pm, ¹⁶⁷Tm, ¹⁷⁵Yb, ¹⁷⁷Lu, ²⁰⁹Bi, ²¹²Pb, ²¹³Po, ²¹³Bi, ²²³Ra, ²²³Fr, ²²⁷Th, ²²⁵Ac, and ²²⁹Th. In some embodiments, the radionuclide is ²²⁵Ac. In some embodiments, the radionuclide is a decay daughter of ²²⁵Ac such as ²²¹Fr, ²¹⁷At, ²¹³Bi, ²¹³Po, ²⁰⁹Tl, ²⁰⁹Pb, or ²⁰⁹Bi. In some embodiments, the radiopharmaceutical conjugate comprises two ²²⁵Ac radionuclides. In some embodiments, the radionuclide is ¹⁷⁷Lu. In some embodiments, the radiopharmaceutical conjugate comprises two ¹⁷⁷Lu radionuclides. In some embodiments, the radionuclide is no-carrier added (i.e., non-carrier-added or n.c.a.) ¹⁷⁷Lu. In some embodiments, the radionuclide is no-carrier added (i.e., non-carrier-added or n.c.a.) ²²⁵Ac. In some embodiments, the radionuclide is ¹⁷⁷Lu free of long-lived radioactive contaminants and byproducts. In some embodiments, the radionuclide is a non-carrier-added radionuclide. In some embodiments, the radionuclide is a pseudo-radiometal. In some embodiments, the pseudo-radiometal is aluminum-[¹⁸F]fluoride ([¹⁸F]AlF) complex.

In some embodiments, the radiopharmaceutical conjugate described herein comprises a beta particle-emitting radionuclide. In some embodiments, the radiopharmaceutical conjugate comprises a beta particle-emitting radionuclide bound to the metal chelator. In some embodiments, the beta particle-emitting radionuclide is copper-67, rhodium-105, ytterbium-175, thulium-167, promethium-153, yttrium-90, samarium-153, or lutetium-177. In some embodiments, the beta particle emitting radionuclide is copper-67, yttrium-90, samarium-153, or lutetium-177. In some embodiments, the beta particle emitting radionuclide is lutetium-177.

In some embodiments, the radiopharmaceutical conjugate described herein comprises a gamma particle-emitting radionuclide. In some embodiments, the radiopharmaceutical conjugate comprises a gamma particle-emitting radionuclide bound to the metal chelator. In some embodiments, the gamma particle-emitting radionuclide is indium-111 or tin-117m.

In some embodiments, the radiopharmaceutical conjugate described herein comprises a positron particle-emitting radionuclide. In some embodiments, the radiopharmaceutical conjugate comprises a positron particle-emitting radionuclide bound to the metal chelator. In some embodiments, the positron-emitting radionuclide is gallium-68, copper-61, copper-62, copper-64, zirconium-89, or terbium-152. In some embodiments, the radionuclide is zirconium-89. In some embodiments, the radionuclide is gallium-68.

In some embodiments, a conjugate described herein comprises a radionuclide suitable for imaging or diagnostic purposes. In some embodiments, the radionuclide suitable for imaging is selected from ⁶²Cu, ⁶⁴Cu, ⁸⁹Zr, ¹³⁴Ce, ¹⁵²Tb, ⁶⁸Ga, ¹¹¹In, and ^{99m}Tc. In some embodiments, the radionuclide is suitable PET imaging. In some embodiments, the radionuclide suitable for PET imaging is selected from ⁶²Cu, ⁶⁴Cu, ⁸⁹Zr, ¹³⁴Ce, ¹⁵²Tb, and ⁶⁸Ga. In some embodiments, the radionuclide is suitable for SPECT imaging. In some embodiments, the radionuclide suitable for SPECT imaging is selected from ¹¹¹In and ^{99m}Tc.

In some embodiments, radiopharmaceutical conjugates described herein do not contain any hot radionuclide, i.e., a cold conjugate. For example, in some cases, a radionuclide can be replaced with a surrogate (e.g., ²²⁵Ac replaced with lanthanum) for testing and experimental purposes. In some embodiments, hot lutetium (Lu-177) is replaced with a cold Lutetium (Lu-175).

A metal chelator such as DOTA can interact with a radionuclide (e.g., ¹⁷⁷Lu or ²²⁵Ac) via one or more functional groups and/or atoms. For example, the metal chelator can interact with a radionuclide via a nitrogen and/or an oxygen atom. As another example, the metal chelator can interact with a radionuclide via carbonyl, carboxylic acid, amino, and/or amide groups of the metal chelator. In some embodiments, the interaction of the metal chelator and a radionuclide of the conjugates disclosed herein can be illustrated as

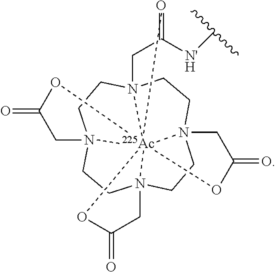

In some embodiments, the interaction of the metal chelator and a radionuclide of the conjugates disclosed herein can be illustrated as

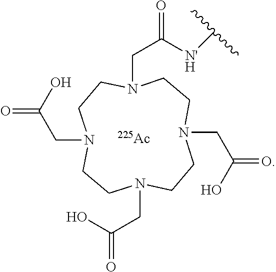

In some embodiments, the interaction of the metal chelator and a radionuclide of the conjugates disclosed herein can be illustrated as

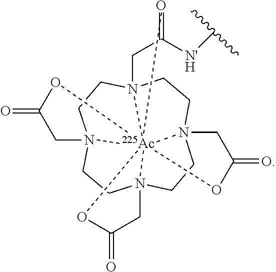

In some embodiments, the interaction of the metal chelator and a radionuclide of the conjugates disclosed herein can be illustrated as

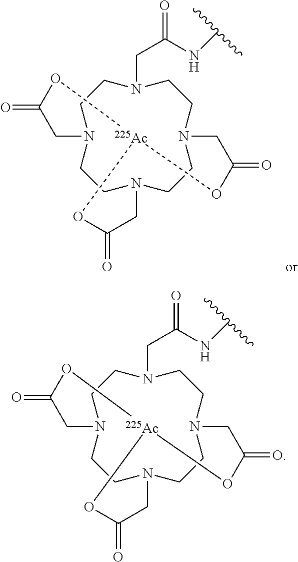

In some embodiments, the interaction of the metal chelator and a radionuclide of the conjugates disclosed herein are illustrated as

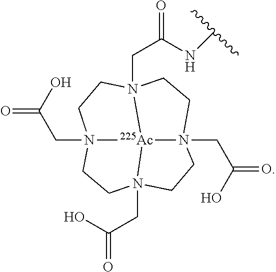

In some embodiments, the interaction of the metal chelator and a radionuclide of the conjugates disclosed herein are illustrated as

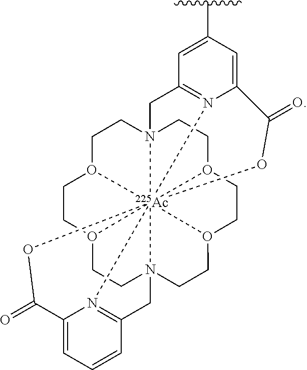

In some embodiments, the radionuclide exists in a positive oxidation state e.g., ²²⁵Ac³⁺, ¹⁷⁷Lu³⁺. In some embodiments, for example in certain aqueous conditions, the radionuclide exists in a salt form, e.g., as ²²⁵Ac³⁺, ¹⁷⁷Lu³⁺. In some embodiments, for example in certain acidic aqueous conditions, the radionuclide exists in a salt form, e.g., as ²²⁵Ac³⁺, ¹⁷⁷Lu³⁺. In some embodiments, the conjugate is in a salt form. In some embodiments, one or more of the carboxylic acid groups of the conjugate may exist as carboxylate anions. In some embodiments, one or more of the carboxylate anions of the conjugate may coordinate to the radionuclide. A person of ordinary skill would appreciate that the dissociation of an acid depends on the pH value of the environment and its pK value. Accordingly, in some embodiments, the conjugate described herein can exist in a completely ionized, partially ionized, or non-ionized form.

In some embodiments, the radiopharmaceutical conjugate is selected from a conjugate found in table 4a or 4b.

TABLE 4a
Radiopharmaceutical conjugate

| Compound | Radionuclide |
|---|---|
| 100 | Ac-225 |
| 100.1 | Ac-225 |
| 100.2 | Ac-225 |
| 100.3 | Ac-225 |
| 100.4 | Ac-225 |
| 100.5 | Ac-225 |
| 100.6 | Ac-225 |
| 100.7 | Ac-225 |
| 100.8 | Ac-225 |
| 100.9 | Ac-225 |
| 100.10 | Ac-225 |
| 100.11 | Ac-225 |
| 100.12 | Ac-225 |
| 100.13 | Ac-225 |
| 100.14 | Ac-225 |
| 100.15 | Ac-225 |
| 100.16 | Ac-225 |
| 100.17 | Ac-225 |
| 100.18 | Ac-225 |
| 100.19 | Ac-225 |
| 101 | Ac-225 |
| 101.1 | Ac-225 |
| 101.2 | Ac-225 |
| 102 | Ac-225 |
| 102.1 | Ac-225 |
| 102.2 | Ac-225 |
| 102.3 | Ac-225 |
| 102.4 | Ac-225 |
| 102.5 | Ac-225 |
| 102.6 | Ac-225 |
| 102.7 | Ac-225 |
| 102.8 | Ac-225 |
| 103 | Ac-225 |
| 103.1 | Ac-225 |
| 103.2 | Ac-225 |
| 103.3 | Ac-225 |
| 103.4 | Ac-225 |
| 104 | Ac-225 |
| 104.1 | Ac-225 |
| 104.2 | Ac-225 |
| 106 | Ac-225 |
| 107 | Ac-225 |
| 107.1 | Ac-225 |
| 107.2 | Ac-225 |
| 107.3 | Ac-225 |
| 107.4 | Ac-225 |
| 107.5 | Ac-225 |
| 107.6 | Ac-225 |
| 107.7 | Ac-225 |
| 107.8 | Ac-225 |
| 107.9 | Ac-225 |
| 107.10 | Ac-225 |
| 107.11 | Ac-225 |
| 107.12 | Ac-225 |
| 107.13 | Ac-225 |
| 107.14 | Ac-225 |
| 107.15 | Ac-225 |
| 107.16 | Ac-225 |
| 107.17 | Ac-225 |
| 107.18 | Ac-225 |
| 107.19 | Ac-225 |
| 107.20 | Ac-225 |
| 107.21 | Ac-225 |
| 107.22 | Ac-225 |
| 107.23 | Ac-225 |
| 107.24 | Ac-225 |
| 107.25 | Ac-225 |
| 107.26 | Ac-225 |
| 107.27 | Ac-225 |
| 107.28 | Ac-225 |
| 107.29 | Ac-225 |
| 107.30 | Ac-225 |
| 107.31 | Ac-225 |
| 107.32 | Ac-225 |
| 107.33 | Ac-225 |
| 107.34 | Ac-225 |
| 107.35 | Ac-225 |
| 107.36 | Ac-225 |
| 107.37 | Ac-225 |
| 107.38 | Ac-225 |
| 107.39 | Ac-225 |
| 107.40 | Ac-225 |
| 107.41 | Ac-225 |
| 107.42 | Ac-225 |
| 107.43 | Ac-225 |
| 107.44 | Ac-225 |
| 107.45 | Ac-225 |
| 107.46 | Ac-225 |
| 107.47 | Ac-225 |
| 107.48 | Ac-225 |
| 107.49 | Ac-225 |
| 107.50 | Ac-225 |
| 107.51 | Ac-225 |
| 107.52 | Ac-225 |
| 107.53 | Ac-225 |
| 107.54 | Ac-225 |
| 107.55 | Ac-225 |
| 107.56 | Ac-225 |
| 107.57 | Ac-225 |
| 107.58 | Ac-225 |
| 107.59 | Ac-225 |
| 107.60 | Ac-225 |
| 107.61 | Ac-225 |
| 108 | Ac-225 |
| 108.1 | Ac-225 |
| 108.2 | Ac-225 |
| 108.3 | Ac-225 |
| 108.4 | Ac-225 |
| 108.5 | Ac-225 |
| 109 | Ac-225 |
| 109.1 | Ac-225 |
| 109.2 | Ac-225 |
| 109.3 | Ac-225 |
| 109.4 | Ac-225 |
| 109.5 | Ac-225 |
| 109.6 | Ac-225 |
| 109.7 | Ac-225 |
| 109.8 | Ac-225 |
| 109.9 | Ac-225 |
| 109.10 | Ac-225 |
| 109.11 | Ac-225 |
| 109.12 | Ac-225 |
| 109.13 | Ac-225 |
| 109.14 | Ac-225 |
| 109.15 | Ac-225 |
| 109.16 | Ac-225 |
| 109.17 | Ac-225 |
| 109.18 | Ac-225 |
| 109.19 | Ac-225 |
| 109.20 | Ac-225 |
| 109.21 | Ac-225 |
| 109.22 | Ac-225 |
| 109.23 | Ac-225 |
| 109.24 | Ac-225 |
| 109.25 | Ac-225 |
| 109.26 | Ac-225 |
| 109.27 | Ac-225 |
| 109.28 | Ac-225 |
| 109.29 | Ac-225 |
| 109.30 | Ac-225 |
| 109.31 | Ac-225 |
| 109.32 | Ac-225 |
| 109.33 | Ac-225 |
| 109.34 | Ac-225 |
| 109.35 | Ac-225 |
| 109.36 | Ac-225 |
| 109.37 | Ac-225 |
| 109.38 | Ac-225 |
| 109.39 | Ac-225 |
| 109.40 | Ac-225 |
| 109.41 | Ac-225 |
| 109.42 | Ac-225 |
| 109.43 | Ac-225 |
| 109.44 | Ac-225 |
| 109.45 | Ac-225 |
| 109.46 | Ac-225 |
| 109.47 | Ac-225 |
| 109.48 | Ac-225 |
| 109.49 | Ac-225 |
| 109.50 | Ac-225 |
| 109.51 | Ac-225 |
| 109.52 | Ac-225 |
| 109.53 | Ac-225 |
| 109.54 | Ac-225 |
| 109.55 | Ac-225 |
| 109.56 | Ac-225 |
| 109.57 | Ac-225 |
| 109.58 | Ac-225 |
| 109.59 | Ac-225 |
| 109.60 | Ac-225 |
| 109.61 | Ac-225 |
| 109.62 | Ac-225 |
| 109.63 | Ac-225 |
| 110 | Ac-225 |
| 111 | Ac-225 |
| 111.1 | Ac-225 |
| 111.2 | Ac-225 |
| 111.3 | Ac-225 |
| 111.4 | Ac-225 |
| 111.5 | Ac-225 |
| 111.6 | Ac-225 |
| 111.7 | Ac-225 |
| 111.8 | Ac-225 |
| 111.9 | Ac-225 |
| 111.10 | Ac-225 |
| 111.11 | Ac-225 |
| 111.12 | Ac-225 |
| 112 | Ac-225 |
| 112.1 | Ac-225 |
| 112.2 | Ac-225 |
| 112.3 | Ac-225 |
| 112.4 | Ac-225 |
| 112.5 | Ac-225 |
| 112.6 | Ac-225 |
| 112.7 | Ac-225 |
| 112.8 | Ac-225 |
| 112.9 | Ac-225 |
| 112.10 | Ac-225 |
| 112.11 | Ac-225 |
| 112.12 | Ac-225 |
| 112.13 | Ac-225 |
| 112.14 | Ac-225 |
| 112.15 | Ac-225 |
| 112.16 | Ac-225 |
| 112.17 | Ac-225 |
| 112.18 | Ac-225 |
| 112.19 | Ac-225 |
| 113 | Ac-225 |
| 114 | Ac-225 |
| 114.1 | Ac-225 |
| 114.2 | Ac-225 |
| 114.3 | Ac-225 |
| 115 | Ac-225 |
| 116 | Ac-225 |
| 116.1 | Ac-225 |
| 116.2 | Ac-225 |
| 116.3 | Ac-225 |
| 116.4 | Ac-225 |
| 117 | Ac-225 |
| 117.1 | Ac-225 |
| 117.2 | Ac-225 |
| 117.3 | Ac-225 |
| 117.4 | Ac-225 |
| 117.5 | Ac-225 |
| 117.6 | Ac-225 |
| 117.7 | Ac-225 |
| 117.8 | Ac-225 |
| 118 | Ac-225 |
| 118.1 | Ac-225 |
| 118.2 | Ac-225 |
| 118.3 | Ac-225 |
| 118.4 | Ac-225 |
| 119 | Ac-225 |
| 120 | Ac-225 |
| 121 | Ac-225 |
| 121.1 | Ac-225 |
| 122 | Ac-225 |
| 123 | Ac-225 |
| 124 | Ac-225 |
| 125 | Ac-225 |
| 125.1 | Ac-225 |
| 126 | Ac-225 |
| 126.1 | Ac-225 |
| 126.2 | Ac-225 |
| 126.3 | Ac-225 |
| 126.4 | Ac-225 |
| 126.5 | Ac-225 |
| 127 | Ac-225 |
| 127.1 | Ac-225 |
| 127.2 | Ac-225 |
| 127.3 | Ac-225 |
| 127.4 | Ac-225 |
| 127.5 | Ac-225 |
| 127.6 | Ac-225 |
| 127.7 | Ac-225 |
| 127.8 | Ac-225 |
| 127.9 | Ac-225 |
| 128 | Ac-225 |
| 128.1 | Ac-225 |
| 129 | Ac-225 |
| 129.1 | Ac-225 |
| 129.2 | Ac-225 |
| 129.3 | Ac-225 |
| 129.4 | Ac-225 |
| 129.5 | Ac-225 |
| 129.6 | Ac-225 |
| 129.7 | Ac-225 |
| 130 | Ac-225 |
| 131 | Ac-225 |
| 131.1 | Ac-225 |
| 131.2 | Ac-225 |
| 131.3 | Ac-225 |
| 131.4 | Ac-225 |
| 131.5 | Ac-225 |
| 131.6 | Ac-225 |
| 131.7 | Ac-225 |
| 132 | Ac-225 |
| 133 | Ac-225 |
| 134 | Ac-225 |
| 135 | Ac-225 |
| 136 | Ac-225 |

TABLE 4b
Radiopharmaceutical conjugate

| Compound | Radionuclide |
|---|---|
| 100 | Lu-177 |
| 100.1 | Lu-177 |
| 100.2 | Lu-177 |
| 100.3 | Lu-177 |
| 100.4 | Lu-177 |
| 100.5 | Lu-177 |
| 100.6 | Lu-177 |
| 100.7 | Lu-177 |
| 100.8 | Lu-177 |
| 100.9 | Lu-177 |
| 100.10 | Lu-177 |
| 100.11 | Lu-177 |
| 100.12 | Lu-177 |
| 100.13 | Lu-177 |
| 100.14 | Lu-177 |
| 100.15 | Lu-177 |
| 100.16 | Lu-177 |
| 100.17 | Lu-177 |
| 100.18 | Lu-177 |
| 100.19 | Lu-177 |
| 101 | Lu-177 |
| 101.1 | Lu-177 |
| 101.2 | Lu-177 |
| 102 | Lu-177 |
| 102.1 | Lu-177 |
| 102.2 | Lu-177 |
| 102.3 | Lu-177 |
| 102.4 | Lu-177 |
| 102.5 | Lu-177 |
| 102.6 | Lu-177 |
| 102.7 | Lu-177 |
| 102.8 | Lu-177 |
| 103 | Lu-177 |
| 103.1 | Lu-177 |
| 103.2 | Lu-177 |
| 103.3 | Lu-177 |
| 103.4 | Lu-177 |
| 104 | Lu-177 |
| 104.1 | Lu-177 |
| 104.2 | Lu-177 |
| 106 | Lu-177 |
| 107 | Lu-177 |
| 107.1 | Lu-177 |
| 107.2 | Lu-177 |
| 107.3 | Lu-177 |
| 107.4 | Lu-177 |
| 107.5 | Lu-177 |
| 107.6 | Lu-177 |
| 107.7 | Lu-177 |
| 107.8 | Lu-177 |
| 107.9 | Lu-177 |
| 107.10 | Lu-177 |
| 107.11 | Lu-177 |
| 107.12 | Lu-177 |
| 107.13 | Lu-177 |
| 107.14 | Lu-177 |
| 107.15 | Lu-177 |
| 107.16 | Lu-177 |
| 107.17 | Lu-177 |
| 107.18 | Lu-177 |
| 107.19 | Lu-177 |
| 107.20 | Lu-177 |
| 107.21 | Lu-177 |
| 107.22 | Lu-177 |
| 107.23 | Lu-177 |
| 107.24 | Lu-177 |
| 107.25 | Lu-177 |
| 107.26 | Lu-177 |
| 107.27 | Lu-177 |
| 107.28 | Lu-177 |
| 107.29 | Lu-177 |
| 107.30 | Lu-177 |
| 107.31 | Lu-177 |
| 107.32 | Lu-177 |
| 107.33 | Lu-177 |
| 107.34 | Lu-177 |
| 107.35 | Lu-177 |
| 107.36 | Lu-177 |
| 107.37 | Lu-177 |
| 107.38 | Lu-177 |
| 107.39 | Lu-177 |
| 107.40 | Lu-177 |
| 107.41 | Lu-177 |
| 107.42 | Lu-177 |
| 107.43 | Lu-177 |
| 107.44 | Lu-177 |
| 107.45 | Lu-177 |
| 107.46 | Lu-177 |
| 107.47 | Lu-177 |
| 107.48 | Lu-177 |
| 107.49 | Lu-177 |
| 107.50 | Lu-177 |
| 107.51 | Lu-177 |
| 107.52 | Lu-177 |
| 107.53 | Lu-177 |
| 107.54 | Lu-177 |
| 107.55 | Lu-177 |
| 107.56 | Lu-177 |
| 107.57 | Lu-177 |
| 107.58 | Lu-177 |
| 107.59 | Lu-177 |
| 107.60 | Lu-177 |
| 107.61 | Lu-177 |
| 108 | Lu-177 |
| 108.1 | Lu-177 |
| 108.2 | Lu-177 |
| 108.3 | Lu-177 |
| 108.4 | Lu-177 |
| 108.5 | Lu-177 |
| 109 | Lu-177 |
| 109.1 | Lu-177 |
| 109.2 | Lu-177 |
| 109.3 | Lu-177 |
| 109.4 | Lu-177 |
| 109.5 | Lu-177 |
| 109.6 | Lu-177 |
| 109.7 | Lu-177 |
| 109.8 | Lu-177 |
| 109.9 | Lu-177 |
| 109.10 | Lu-177 |
| 109.11 | Lu-177 |
| 109.12 | Lu-177 |
| 109.13 | Lu-177 |
| 109.14 | Lu-177 |
| 109.15 | Lu-177 |
| 109.16 | Lu-177 |
| 109.17 | Lu-177 |
| 109.18 | Lu-177 |
| 109.19 | Lu-177 |
| 109.20 | Lu-177 |
| 109.21 | Lu-177 |
| 109.22 | Lu-177 |
| 109.23 | Lu-177 |
| 109.24 | Lu-177 |
| 109.25 | Lu-177 |
| 109.26 | Lu-177 |
| 109.27 | Lu-177 |
| 109.28 | Lu-177 |
| 109.29 | Lu-177 |
| 109.30 | Lu-177 |
| 109.31 | Lu-177 |
| 109.32 | Lu-177 |
| 109.33 | Lu-177 |
| 109.34 | Lu-177 |
| 109.35 | Lu-177 |
| 109.36 | Lu-177 |
| 109.37 | Lu-177 |
| 109.38 | Lu-177 |
| 109.39 | Lu-177 |
| 109.40 | Lu-177 |
| 109.41 | Lu-177 |
| 109.42 | Lu-177 |
| 109.43 | Lu-177 |
| 109.44 | Lu-177 |
| 109.45 | Lu-177 |
| 109.46 | Lu-177 |
| 109.47 | Lu-177 |
| 109.48 | Lu-177 |
| 109.49 | Lu-177 |
| 109.50 | Lu-177 |
| 109.51 | Lu-177 |
| 109.52 | Lu-177 |
| 109.53 | Lu-177 |
| 109.54 | Lu-177 |
| 109.55 | Lu-177 |
| 109.56 | Lu-177 |
| 109.57 | Lu-177 |
| 109.58 | Lu-177 |
| 109.59 | Lu-177 |
| 109.60 | Lu-177 |
| 109.61 | Lu-177 |
| 109.62 | Lu-177 |
| 109.63 | Lu-177 |
| 110 | Lu-177 |
| 111 | Lu-177 |
| 111.1 | Lu-177 |
| 111.2 | Lu-177 |
| 111.3 | Lu-177 |
| 111.4 | Lu-177 |
| 111.5 | Lu-177 |
| 111.6 | Lu-177 |
| 111.7 | Lu-177 |
| 111.8 | Lu-177 |
| 111.9 | Lu-177 |
| 111.10 | Lu-177 |
| 111.11 | Lu-177 |
| 111.12 | Lu-177 |
| 112 | Lu-177 |
| 112.1 | Lu-177 |
| 112.2 | Lu-177 |
| 112.3 | Lu-177 |
| 112.4 | Lu-177 |
| 112.5 | Lu-177 |
| 112.6 | Lu-177 |
| 112.7 | Lu-177 |
| 112.8 | Lu-177 |
| 112.9 | Lu-177 |
| 112.10 | Lu-177 |
| 112.11 | Lu-177 |
| 112.12 | Lu-177 |
| 112.13 | Lu-177 |
| 112.14 | Lu-177 |
| 112.15 | Lu-177 |
| 112.16 | Lu-177 |
| 112.17 | Lu-177 |
| 112.18 | Lu-177 |
| 112.19 | Lu-177 |
| 113 | Lu-177 |
| 114 | Lu-177 |
| 114.1 | Lu-177 |
| 114.2 | Lu-177 |
| 114.3 | Lu-177 |
| 115 | Lu-177 |
| 116 | Lu-177 |
| 116.1 | Lu-177 |
| 116.2 | Lu-177 |
| 116.3 | Lu-177 |
| 116.4 | Lu-177 |
| 117 | Lu-177 |
| 117.1 | Lu-177 |
| 117.2 | Lu-177 |
| 117.3 | Lu-177 |
| 117.4 | Lu-177 |
| 117.5 | Lu-177 |
| 117.6 | Lu-177 |
| 117.7 | Lu-177 |
| 117.8 | Lu-177 |
| 118 | Lu-177 |
| 118.1 | Lu-177 |
| 118.2 | Lu-177 |
| 118.3 | Lu-177 |
| 118.4 | Lu-177 |
| 119 | Lu-177 |
| 120 | Lu-177 |
| 121 | Lu-177 |
| 121.1 | Lu-177 |
| 122 | Lu-177 |
| 123 | Lu-177 |
| 124 | Lu-177 |
| 125 | Lu-177 |
| 125.1 | Lu-177 |
| 126 | Lu-177 |
| 126.1 | Lu-177 |
| 126.2 | Lu-177 |
| 126.3 | Lu-177 |
| 126.4 | Lu-177 |
| 126.5 | Lu-177 |
| 127 | Lu-177 |
| 127.1 | Lu-177 |
| 127.2 | Lu-177 |
| 127.3 | Lu-177 |
| 127.4 | Lu-177 |
| 127.5 | Lu-177 |
| 127.6 | Lu-177 |
| 127.7 | Lu-177 |
| 127.8 | Lu-177 |
| 127.9 | Lu-177 |
| 128 | Lu-177 |
| 128.1 | Lu-177 |
| 129 | Lu-177 |
| 129.1 | Lu-177 |
| 129.2 | Lu-177 |
| 129.3 | Lu-177 |
| 129.4 | Lu-177 |
| 129.5 | Lu-177 |
| 129.6 | Lu-177 |
| 129.7 | Lu-177 |
| 130 | Lu-177 |
| 131 | Lu-177 |
| 131.1 | Lu-177 |
| 131.2 | Lu-177 |
| 131.3 | Lu-177 |
| 131.4 | Lu-177 |
| 131.5 | Lu-177 |
| 131.6 | Lu-177 |
| 131.7 | Lu-177 |
| 132 | Lu-177 |
| 133 | Lu-177 |
| 134 | Lu-177 |
| 135 | Lu-177 |
| 136 | Lu-177 |

The radionuclide of Table 4a and Table 4b is bound to the chelator of the corresponding compound.

In some embodiments, the radiopharmaceutical conjugate is selected from a conjugate found in table 5a or 5b.

TABLE 5a
Radiopharmaceutical conjugate

| Compound | Radionuclide |
|---|---|
| 100 | Cu-64 |
| 100.1 | Cu-64 |
| 100.2 | Cu-64 |
| 100.3 | Cu-64 |
| 100.4 | Cu-64 |
| 100.5 | Cu-64 |
| 100.6 | Cu-64 |
| 100.7 | Cu-64 |
| 100.8 | Cu-64 |
| 100.9 | Cu-64 |
| 100.10 | Cu-64 |
| 100.11 | Cu-64 |
| 100.12 | Cu-64 |
| 100.13 | Cu-64 |
| 100.14 | Cu-64 |
| 100.15 | Cu-64 |
| 100.16 | Cu-64 |
| 100.17 | Cu-64 |
| 100.18 | Cu-64 |
| 100.19 | Cu-64 |
| 101 | Cu-64 |
| 101.1 | Cu-64 |
| 101.2 | Cu-64 |
| 102 | Cu-64 |
| 102.1 | Cu-64 |
| 102.2 | Cu-64 |
| 102.3 | Cu-64 |
| 102.4 | Cu-64 |
| 102.5 | Cu-64 |
| 102.6 | Cu-64 |
| 102.7 | Cu-64 |
| 102.8 | Cu-64 |
| 103 | Cu-64 |
| 103.1 | Cu-64 |
| 103.2 | Cu-64 |
| 103.3 | Cu-64 |
| 103.4 | Cu-64 |
| 104 | Cu-64 |
| 104.1 | Cu-64 |
| 104.2 | Cu-64 |
| 106 | Cu-64 |
| 107 | Cu-64 |
| 107.1 | Cu-64 |
| 107.2 | Cu-64 |
| 107.3 | Cu-64 |
| 107.4 | Cu-64 |
| 107.5 | Cu-64 |
| 107.6 | Cu-64 |
| 107.7 | Cu-64 |
| 107.8 | Cu-64 |
| 107.9 | Cu-64 |
| 107.10 | Cu-64 |
| 107.11 | Cu-64 |
| 107.12 | Cu-64 |
| 107.13 | Cu-64 |
| 107.14 | Cu-64 |
| 107.15 | Cu-64 |
| 107.16 | Cu-64 |
| 107.17 | Cu-64 |
| 107.18 | Cu-64 |
| 107.19 | Cu-64 |
| 107.20 | Cu-64 |
| 107.21 | Cu-64 |
| 107.22 | Cu-64 |
| 107.23 | Cu-64 |
| 107.24 | Cu-64 |
| 107.25 | Cu-64 |
| 107.26 | Cu-64 |
| 107.27 | Cu-64 |
| 107.28 | Cu-64 |
| 107.29 | Cu-64 |
| 107.30 | Cu-64 |
| 107.31 | Cu-64 |
| 107.32 | Cu-64 |
| 107.33 | Cu-64 |
| 107.34 | Cu-64 |
| 107.35 | Cu-64 |
| 107.36 | Cu-64 |
| 107.37 | Cu-64 |
| 107.38 | Cu-64 |
| 107.39 | Cu-64 |
| 107.40 | Cu-64 |
| 107.41 | Cu-64 |
| 107.42 | Cu-64 |
| 107.43 | Cu-64 |
| 107.44 | Cu-64 |
| 107.45 | Cu-64 |
| 107.46 | Cu-64 |
| 107.47 | Cu-64 |
| 107.48 | Cu-64 |
| 107.49 | Cu-64 |
| 107.50 | Cu-64 |
| 107.51 | Cu-64 |
| 107.52 | Cu-64 |
| 107.53 | Cu-64 |
| 107.54 | Cu-64 |
| 107.55 | Cu-64 |
| 107.56 | Cu-64 |
| 107.57 | Cu-64 |
| 107.58 | Cu-64 |
| 107.59 | Cu-64 |
| 107.60 | Cu-64 |
| 107.61 | Cu-64 |
| 108 | Cu-64 |
| 108.1 | Cu-64 |
| 108.2 | Cu-64 |
| 108.3 | Cu-64 |
| 108.4 | Cu-64 |
| 108.5 | Cu-64 |
| 109 | Cu-64 |
| 109.1 | Cu-64 |
| 109.2 | Cu-64 |
| 109.3 | Cu-64 |
| 109.4 | Cu-64 |
| 109.5 | Cu-64 |
| 109.6 | Cu-64 |
| 109.7 | Cu-64 |
| 109.8 | Cu-64 |
| 109.9 | Cu-64 |
| 109.10 | Cu-64 |
| 109.11 | Cu-64 |
| 109.12 | Cu-64 |
| 109.13 | Cu-64 |
| 109.14 | Cu-64 |
| 109.15 | Cu-64 |
| 109.16 | Cu-64 |
| 109.17 | Cu-64 |
| 109.18 | Cu-64 |
| 109.19 | Cu-64 |
| 109.20 | Cu-64 |
| 109.21 | Cu-64 |
| 109.22 | Cu-64 |
| 109.23 | Cu-64 |
| 109.24 | Cu-64 |
| 109.25 | Cu-64 |
| 109.26 | Cu-64 |
| 109.27 | Cu-64 |
| 109.28 | Cu-64 |
| 109.29 | Cu-64 |
| 109.30 | Cu-64 |
| 109.31 | Cu-64 |
| 109.32 | Cu-64 |
| 109.33 | Cu-64 |
| 109.34 | Cu-64 |
| 109.35 | Cu-64 |
| 109.36 | Cu-64 |
| 109.37 | Cu-64 |
| 109.38 | Cu-64 |
| 109.39 | Cu-64 |
| 109.40 | Cu-64 |
| 109.41 | Cu-64 |
| 109.42 | Cu-64 |
| 109.43 | Cu-64 |
| 109.44 | Cu-64 |
| 109.45 | Cu-64 |
| 109.46 | Cu-64 |
| 109.47 | Cu-64 |
| 109.48 | Cu-64 |
| 109.49 | Cu-64 |
| 109.50 | Cu-64 |
| 109.51 | Cu-64 |
| 109.52 | Cu-64 |
| 109.53 | Cu-64 |
| 109.54 | Cu-64 |
| 109.55 | Cu-64 |
| 109.56 | Cu-64 |
| 109.57 | Cu-64 |
| 109.58 | Cu-64 |
| 109.59 | Cu-64 |
| 109.60 | Cu-64 |
| 109.61 | Cu-64 |
| 109.62 | Cu-64 |
| 109.63 | Cu-64 |
| 110 | Cu-64 |
| 111 | Cu-64 |
| 111.1 | Cu-64 |
| 111.2 | Cu-64 |
| 111.3 | Cu-64 |
| 111.4 | Cu-64 |
| 111.5 | Cu-64 |
| 111.6 | Cu-64 |
| 111.7 | Cu-64 |
| 111.8 | Cu-64 |
| 111.9 | Cu-64 |
| 111.10 | Cu-64 |
| 111.11 | Cu-64 |
| 111.12 | Cu-64 |
| 112 | Cu-64 |
| 112.1 | Cu-64 |
| 112.2 | Cu-64 |
| 112.3 | Cu-64 |
| 112.4 | Cu-64 |
| 112.5 | Cu-64 |
| 112.6 | Cu-64 |
| 112.7 | Cu-64 |
| 112.8 | Cu-64 |
| 112.9 | Cu-64 |
| 112.10 | Cu-64 |
| 112.11 | Cu-64 |
| 112.12 | Cu-64 |
| 112.13 | Cu-64 |
| 112.14 | Cu-64 |
| 112.15 | Cu-64 |
| 112.16 | Cu-64 |
| 112.17 | Cu-64 |
| 112.18 | Cu-64 |
| 112.19 | Cu-64 |
| 113 | Cu-64 |
| 114 | Cu-64 |
| 114.1 | Cu-64 |
| 114.2 | Cu-64 |
| 114.3 | Cu-64 |
| 115 | Cu-64 |
| 116 | Cu-64 |
| 116.1 | Cu-64 |
| 116.2 | Cu-64 |
| 116.3 | Cu-64 |
| 116.4 | Cu-64 |
| 117 | Cu-64 |
| 117.1 | Cu-64 |
| 117.2 | Cu-64 |
| 117.3 | Cu-64 |
| 117.4 | Cu-64 |
| 117.5 | Cu-64 |
| 117.6 | Cu-64 |
| 117.7 | Cu-64 |
| 117.8 | Cu-64 |
| 118 | Cu-64 |
| 118.1 | Cu-64 |
| 118.2 | Cu-64 |
| 118.3 | Cu-64 |
| 118.4 | Cu-64 |
| 119 | Cu-64 |
| 120 | Cu-64 |
| 121 | Cu-64 |
| 121.1 | Cu-64 |
| 122 | Cu-64 |
| 123 | Cu-64 |
| 124 | Cu-64 |
| 125 | Cu-64 |
| 125.1 | Cu-64 |
| 126 | Cu-64 |
| 126.1 | Cu-64 |
| 126.2 | Cu-64 |
| 126.3 | Cu-64 |
| 126.4 | Cu-64 |
| 126.5 | Cu-64 |
| 127 | Cu-64 |
| 127.1 | Cu-64 |
| 127.2 | Cu-64 |
| 127.3 | Cu-64 |
| 127.4 | Cu-64 |
| 127.5 | Cu-64 |
| 127.6 | Cu-64 |
| 127.7 | Cu-64 |
| 127.8 | Cu-64 |
| 127.9 | Cu-64 |
| 128 | Cu-64 |
| 128.1 | Cu-64 |
| 129 | Cu-64 |
| 129.1 | Cu-64 |
| 129.2 | Cu-64 |
| 129.3 | Cu-64 |
| 129.4 | Cu-64 |
| 129.5 | Cu-64 |
| 129.6 | Cu-64 |
| 129.7 | Cu-64 |
| 130 | Cu-64 |
| 131 | Cu-64 |
| 131.1 | Cu-64 |
| 131.2 | Cu-64 |
| 131.3 | Cu-64 |
| 131.4 | Cu-64 |
| 131.5 | Cu-64 |
| 131.6 | Cu-64 |
| 131.7 | Cu-64 |
| 132 | Cu-64 |
| 133 | Cu-64 |
| 134 | Cu-64 |
| 135 | Cu-64 |
| 136 | Cu-64 |

TABLE 5b
Radiopharmaceutical conjugate

| Compound | Radionuclide |
|---|---|
| 100 | Ga-68 |
| 100.1 | Ga-68 |
| 100.2 | Ga-68 |
| 100.3 | Ga-68 |
| 100.4 | Ga-68 |
| 100.5 | Ga-68 |
| 100.6 | Ga-68 |
| 100.7 | Ga-68 |
| 100.8 | Ga-68 |
| 100.9 | Ga-68 |
| 100.10 | Ga-68 |
| 100.11 | Ga-68 |
| 100.12 | Ga-68 |
| 100.13 | Ga-68 |
| 100.14 | Ga-68 |
| 100.15 | Ga-68 |
| 100.16 | Ga-68 |
| 100.17 | Ga-68 |
| 100.18 | Ga-68 |
| 100.19 | Ga-68 |
| 101 | Ga-68 |
| 101.1 | Ga-68 |
| 101.2 | Ga-68 |
| 102 | Ga-68 |
| 102.1 | Ga-68 |
| 102.2 | Ga-68 |
| 102.3 | Ga-68 |
| 102.4 | Ga-68 |
| 102.5 | Ga-68 |
| 102.6 | Ga-68 |
| 102.7 | Ga-68 |
| 102.8 | Ga-68 |
| 103 | Ga-68 |
| 103.1 | Ga-68 |
| 103.2 | Ga-68 |
| 103.3 | Ga-68 |
| 103.4 | Ga-68 |
| 104 | Ga-68 |
| 104.1 | Ga-68 |
| 104.2 | Ga-68 |
| 106 | Ga-68 |
| 107 | Ga-68 |
| 107.1 | Ga-68 |
| 107.2 | Ga-68 |
| 107.3 | Ga-68 |
| 107.4 | Ga-68 |
| 107.5 | Ga-68 |
| 107.6 | Ga-68 |
| 107.7 | Ga-68 |
| 107.8 | Ga-68 |
| 107.9 | Ga-68 |
| 107.10 | Ga-68 |
| 107.11 | Ga-68 |
| 107.12 | Ga-68 |
| 107.13 | Ga-68 |
| 107.14 | Ga-68 |
| 107.15 | Ga-68 |
| 107.16 | Ga-68 |
| 107.17 | Ga-68 |
| 107.18 | Ga-68 |
| 107.19 | Ga-68 |
| 107.20 | Ga-68 |
| 107.21 | Ga-68 |
| 107.22 | Ga-68 |
| 107.23 | Ga-68 |
| 107.24 | Ga-68 |
| 107.25 | Ga-68 |
| 107.26 | Ga-68 |
| 107.27 | Ga-68 |
| 107.28 | Ga-68 |
| 107.29 | Ga-68 |
| 107.30 | Ga-68 |
| 107.31 | Ga-68 |
| 107.32 | Ga-68 |
| 107.33 | Ga-68 |
| 107.34 | Ga-68 |
| 107.35 | Ga-68 |
| 107.36 | Ga-68 |
| 107.37 | Ga-68 |
| 107.38 | Ga-68 |
| 107.39 | Ga-68 |
| 107.40 | Ga-68 |
| 107.41 | Ga-68 |
| 107.42 | Ga-68 |
| 107.43 | Ga-68 |
| 107.44 | Ga-68 |
| 107.45 | Ga-68 |
| 107.46 | Ga-68 |
| 107.47 | Ga-68 |
| 107.48 | Ga-68 |
| 107.49 | Ga-68 |
| 107.50 | Ga-68 |
| 107.51 | Ga-68 |
| 107.52 | Ga-68 |
| 107.53 | Ga-68 |
| 107.54 | Ga-68 |
| 107.55 | Ga-68 |
| 107.56 | Ga-68 |
| 107.57 | Ga-68 |
| 107.58 | Ga-68 |
| 107.59 | Ga-68 |
| 107.60 | Ga-68 |
| 107.61 | Ga-68 |
| 108 | Ga-68 |
| 108.1 | Ga-68 |
| 108.2 | Ga-68 |
| 108.3 | Ga-68 |
| 108.4 | Ga-68 |
| 108.5 | Ga-68 |
| 109 | Ga-68 |
| 109.1 | Ga-68 |
| 109.2 | Ga-68 |
| 109.3 | Ga-68 |
| 109.4 | Ga-68 |
| 109.5 | Ga-68 |
| 109.6 | Ga-68 |
| 109.7 | Ga-68 |
| 109.8 | Ga-68 |
| 109.9 | Ga-68 |
| 109.10 | Ga-68 |
| 109.11 | Ga-68 |
| 109.12 | Ga-68 |
| 109.13 | Ga-68 |
| 109.14 | Ga-68 |
| 109.15 | Ga-68 |
| 109.16 | Ga-68 |
| 109.17 | Ga-68 |
| 109.18 | Ga-68 |
| 109.19 | Ga-68 |
| 109.20 | Ga-68 |
| 109.21 | Ga-68 |
| 109.22 | Ga-68 |
| 109.23 | Ga-68 |
| 109.24 | Ga-68 |
| 109.25 | Ga-68 |
| 109.26 | Ga-68 |
| 109.27 | Ga-68 |
| 109.28 | Ga-68 |
| 109.29 | Ga-68 |
| 109.30 | Ga-68 |
| 109.31 | Ga-68 |
| 109.32 | Ga-68 |
| 109.33 | Ga-68 |
| 109.34 | Ga-68 |
| 109.35 | Ga-68 |
| 109.36 | Ga-68 |
| 109.37 | Ga-68 |
| 109.38 | Ga-68 |
| 109.39 | Ga-68 |
| 109.40 | Ga-68 |
| 109.41 | Ga-68 |
| 109.42 | Ga-68 |
| 109.43 | Ga-68 |
| 109.44 | Ga-68 |
| 109.45 | Ga-68 |
| 109.46 | Ga-68 |
| 109.47 | Ga-68 |
| 109.48 | Ga-68 |
| 109.49 | Ga-68 |
| 109.50 | Ga-68 |
| 109.51 | Ga-68 |
| 109.52 | Ga-68 |
| 109.53 | Ga-68 |
| 109.54 | Ga-68 |
| 109.55 | Ga-68 |
| 109.56 | Ga-68 |
| 109.57 | Ga-68 |
| 109.58 | Ga-68 |
| 109.59 | Ga-68 |
| 109.60 | Ga-68 |
| 109.61 | Ga-68 |
| 109.62 | Ga-68 |
| 109.63 | Ga-68 |
| 110 | Ga-68 |
| 111 | Ga-68 |
| 111.1 | Ga-68 |
| 111.2 | Ga-68 |
| 111.3 | Ga-68 |
| 111.4 | Ga-68 |
| 111.5 | Ga-68 |
| 111.6 | Ga-68 |
| 111.7 | Ga-68 |
| 111.8 | Ga-68 |
| 111.9 | Ga-68 |
| 111.10 | Ga-68 |
| 111.11 | Ga-68 |
| 111.12 | Ga-68 |
| 112 | Ga-68 |
| 112.1 | Ga-68 |
| 112.2 | Ga-68 |
| 112.3 | Ga-68 |
| 112.4 | Ga-68 |
| 112.5 | Ga-68 |
| 112.6 | Ga-68 |
| 112.7 | Ga-68 |
| 112.8 | Ga-68 |
| 112.9 | Ga-68 |
| 112.10 | Ga-68 |
| 112.11 | Ga-68 |
| 112.12 | Ga-68 |
| 112.13 | Ga-68 |
| 112.14 | Ga-68 |
| 112.15 | Ga-68 |
| 112.16 | Ga-68 |
| 112.17 | Ga-68 |
| 112.18 | Ga-68 |
| 112.19 | Ga-68 |
| 113 | Ga-68 |
| 114 | Ga-68 |
| 114.1 | Ga-68 |
| 114.2 | Ga-68 |
| 114.3 | Ga-68 |
| 115 | Ga-68 |
| 116 | Ga-68 |
| 116.1 | Ga-68 |
| 116.2 | Ga-68 |
| 116.3 | Ga-68 |
| 116.4 | Ga-68 |
| 117 | Ga-68 |
| 117.1 | Ga-68 |
| 117.2 | Ga-68 |
| 117.3 | Ga-68 |
| 117.4 | Ga-68 |
| 117.5 | Ga-68 |
| 117.6 | Ga-68 |
| 117.7 | Ga-68 |
| 117.8 | Ga-68 |
| 118 | Ga-68 |
| 118.1 | Ga-68 |
| 118.2 | Ga-68 |
| 118.3 | Ga-68 |
| 118.4 | Ga-68 |
| 119 | Ga-68 |
| 120 | Ga-68 |
| 121 | Ga-68 |
| 121.1 | Ga-68 |
| 122 | Ga-68 |
| 123 | Ga-68 |
| 124 | Ga-68 |
| 125 | Ga-68 |
| 125.1 | Ga-68 |
| 126 | Ga-68 |
| 126.1 | Ga-68 |
| 126.2 | Ga-68 |
| 126.3 | Ga-68 |
| 126.4 | Ga-68 |
| 126.5 | Ga-68 |
| 127 | Ga-68 |
| 127.1 | Ga-68 |
| 127.2 | Ga-68 |
| 127.3 | Ga-68 |
| 127.4 | Ga-68 |
| 127.5 | Ga-68 |
| 127.6 | Ga-68 |
| 127.7 | Ga-68 |
| 127.8 | Ga-68 |
| 127.9 | Ga-68 |
| 128 | Ga-68 |
| 128.1 | Ga-68 |
| 129 | Ga-68 |
| 129.1 | Ga-68 |
| 129.2 | Ga-68 |
| 129.3 | Ga-68 |
| 129.4 | Ga-68 |
| 129.5 | Ga-68 |
| 129.6 | Ga-68 |
| 129.7 | Ga-68 |
| 130 | Ga-68 |
| 131 | Ga-68 |
| 131.1 | Ga-68 |
| 131.2 | Ga-68 |
| 131.3 | Ga-68 |
| 131.4 | Ga-68 |
| 131.5 | Ga-68 |
| 131.6 | Ga-68 |
| 131.7 | Ga-68 |
| 132 | Ga-68 |
| 133 | Ga-68 |
| 134 | Ga-68 |
| 135 | Ga-68 |
| 136 | Ga-68 |

The radionuclide of Table 5a and Table 5b is bound to the chelator of the corresponding compound.

In some embodiments, disclosed herein is a radiopharmaceutical conjugate comprising a covalently bound radioisotope. In some embodiments of Formula (AC), (AC′), (BC), (BC′), (CC), or (CC′), R³⁰ comprises the covalently bound radioisotope. In some embodiments of Formula (AC), (BC), or (CC), TL₁ comprises the covalently bound radioisotope. In some embodiments of Formula (AC), (BC), or (CC), TL₂ comprises the covalently bound radioisotope. In some embodiments of Formula (AC), (BC), or (CC), L^{A} comprises the covalently bound radioisotope. In some embodiments of Formula (AC′), (BC′), or (CC′), one or both of the TL₁ groups comprise the covalently bound radioisotope. In some embodiments of Formula (AC′), (BC′), or (CC′), one or both of the TL₂ groups comprise the covalently bound radioisotope. In some embodiments of Formula (AC′), (BC′), or (CC′), L^{B} comprises the covalently bound radioisotope. In some embodiments of Formula (BC), (BC′), (CC), or (CC′), X comprises the covalently bound radioisotope. In some embodiments, the covalently bound radioisotope is selected from ¹¹C, ¹³N, ¹⁵O, ¹⁸F, ⁷⁰As, ⁷¹As, ⁷²As, ⁷³As, ⁷⁴As, ⁷⁶As, ⁷⁷As, ⁷⁶Br, ¹²³I, ¹²⁴I, ¹²⁵I, ¹³¹I, and ²¹¹At. In some embodiments, the covalently bound radioisotope is ¹³¹I. In some embodiments, the covalently bound radioisotope is ²¹¹At. In some embodiments, the covalently bound radioisotope is ¹⁸F.

In some embodiments, a compound of Formula (AC), (AC′), (BC), (BC′), (CC), or (CC′), or a pharmaceutically acceptable salt or a stereoisomer thereof comprises a covalently bound radioisotope (designated as R*), and wherein the compound comprises a structure represented in Formula (Va), Formula (Vb), Formula (Vc), Formula (Vd), or Formula (Ve):

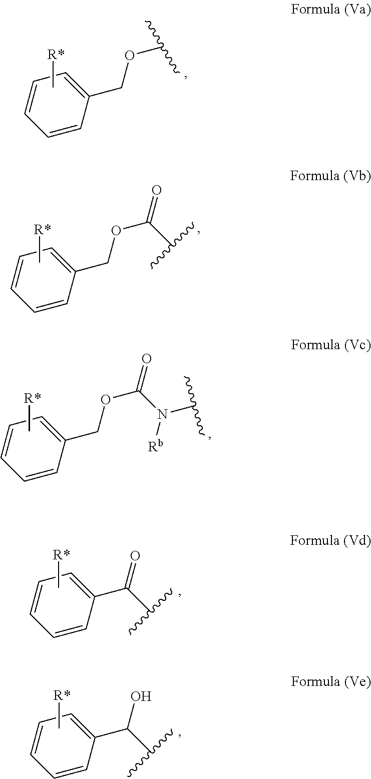

wherein,
R* is a covalently bound radioisotope (e.g., such as fluorine-18 (¹⁸F), iodine-131 (¹³¹I), iodine-123 (¹²³I), iodine-124 (¹²⁴I), iodine-125 (¹²⁵I), or astatine-211 (²¹¹At)); and
R^{b} is hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, C₃-C₆cycloalkyl, C₂-C₆heterocycloalkyl, aryl, or heteroaryl, wherein each of the alkyl, alkenyl, alkynyl, cycloalkyl, heterocycloalkyl, aryl, or heteroaryl is optionally substituted (e.g., optionally substituted with one or more R as described herein).

In some embodiments of Formula (AC), (AC′), (BC), (BC′), (CC), or (CC′), R³⁰ comprises a structure represented in Formula (Va), Formula (Vb), Formula (Vc), Formula (Vd), or Formula (Ve).

In some embodiments of Formula (Va), Formula (Vb), Formula (Vc), Formula (Vd), or Formula (Ve), the alkyl, haloalkyl, hydroxyalkyl, aminoalkyl, heteroalkyl, alkenyl, alkynyl, cycloalkyl, heterocycloalkyl, aryl, or heteroaryl are independently optionally substituted by one or more halogen, amino, —OH, —NO₂, oxo, —CN, C₁₋₃ alkoxyl, C₁₋₃ alkyl and C₁₋₃ haloalkyl. In some embodiments, R^{b} is hydrogen. In some embodiments, R^{b} is C₁-C₄alkyl. In some embodiments, R^{b} is C₁-C₄cycloalkyl. In some embodiments, R* is ¹³¹I.

In some embodiments, a structure represented in Formula (Va), Formula (Vb), Formula (Vc), Formula (Vd), and Formula (Ve) is selected from the following:

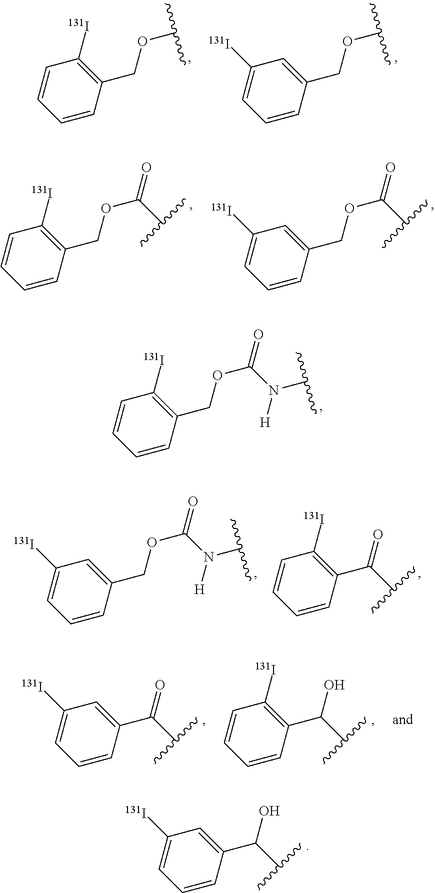

In some embodiments, radiopharmaceutical conjugates comprising covalently bound radionuclides described herein can be synthesized from a boronic acid, boronate, or stannane precursor. Boronic acid, boronate, and stannane precursors can be formed according to the following general reaction:

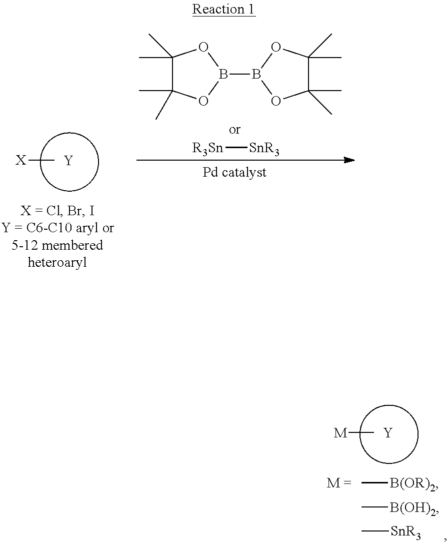

where R is a variable chemical moiety, for example alkyl or hydrogen. A halogen on ring Y, for example chloro, bromo, or iodo, can undergo a palladium mediated coupling reaction with a boronic acid, boronate, or stannane compound to form an intermediate compound wherein group M has replaced halogen X.

A radioisotope, for example R*, such as fluorine-18 (¹⁸F), iodine-131 (¹³¹I), iodine-123 (¹²³I), iodine-124 (¹²⁴I), iodine-125 (¹²⁵I), or astatine-211 (²¹¹At), or any other radioisotope of Table 5A and Table 5B can be formed, for example, from the intermediate compound according to the following general reactions:

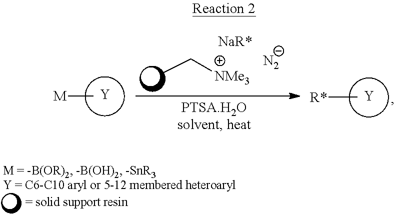

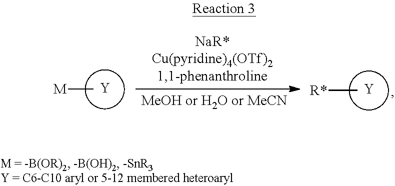

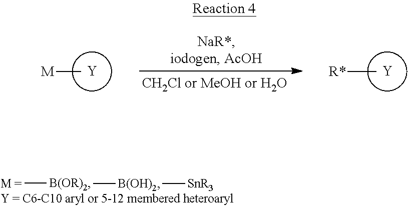

where R is a variable chemical moiety, for example alkyl or hydrogen.

In some embodiments, radiolabeled conjugates described herein can be synthesized from a chloro, bromo, or iodio precursor according to the following general reaction:

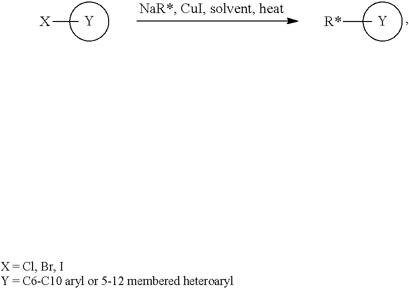

where R* is a radioisotope, for example, fluorine-18 (¹⁸F), iodine-131 (¹³¹I), iodine-123 (¹²³I), iodine-124 (¹²⁴I), iodine-125 (¹²⁵I), astatine-211 (²¹¹At), or a radioisotope of Table 5A and Table 5B.

The radiolabeling reactions depicted above are used as example procedures in the synthesis of radiolabeled conjugates described herein. Additional reactions, including nucleophilic substitution, electrophilic substitution, isotopic exchanges, bromine-radioiodine exchange, radioiododestannylation, radioiododeboronation, and transition metal mediated halogen exchange are contemplated and procedures can be found in Berdal et al., “Investigation on the reactivity of nucleophilic radiohalogens with arylboronic acids in water: access to an efficient single-step method for the radioiodination and astatination of antibodies” Chemical Science 2021, 12, 1458, and Dubost et al., “Recent Advances in Synthetic Methods for Radioiodination” J. Org. Chem, 2020, 85, 13, 8300-8310, both of which are incorporated by reference herein in their entirety.

### Method of Treatment

Disclosed herein are methods of treating a CA-IX-mediated disease or disorder in a subject in need thereof, the method comprising administering to the subject a radiopharmaceutical conjugate disclosed herein. In some embodiments, the CA-IX-mediated disease or disorder is cancer.

Disclosed herein are methods of treating cancer in a subject in need thereof, the method comprising administering to the subject a radiopharmaceutical conjugate disclosed herein.

In some embodiments, the cancer is a solid tumor. In some embodiments, the cancer is a relapsed or refractory cancer. In some embodiments, the cancer is a metastatic cancer. In some embodiments, the cancer is hypoxic.

In some embodiments, the cancer includes any of various malignant neoplasms characterized by the proliferation of anaplastic cells that tend to invade surrounding tissue and metastasize to new body sites. Non-limiting examples of cancers suitable for treatment using the radiopharmaceutical conjugate disclosed herein include anal cancer, appendix cancer, bile duct cancer, bladder cancer, bone cancer, breast cancer, cancer of the central nervous system, cervical cancer, choriocarcinomas, colon cancer, colorectal cancer, esophageal cancer, fibrosarcoma, gall bladder cancer, gastrointestinal carcinoid tumors, glioma, head and neck cancers, leukemia (e.g., acute lymphocytic leukemia, chronic lymphocytic leukemia, acute myelogenous leukemia, chronic myelogenous leukemia, or hairy cell leukemia), liver cancer, lung cancer, lymphoma (e.g., non-Hodgkin's lymphoma, Hodgkin's lymphoma, B-cell lymphoma, or Burkitt's lymphoma), multiple myeloma, neuroblastoma, oral squamous cell carcinoma, osteogenic sarcomas, ovarian cancer, pancreatic cancer, pleural cancer, prostate cancer, rectal cancer, renal cancer (e.g., renal cell carcinoma), skin cancer (e.g., melanoma) small intestine cancer, stomach (gastric) cancer, testicular cancer, and thyroid cancer.

In some embodiments, the cancer is clear cell renal cell carcinoma (ccRCC), endometrial cancer, basal cell carcinoma, liver cancer, colon cancer, gastric cancer, cervical squamous carcinoma, oral squamous cell carcinoma, pancreatic cancer, breast cancer, ovarian cancer, glioblastoma, head and neck cancer, or lung cancer. In some embodiments, the cancer is clear cell renal cell carcinoma (ccRCC). In some embodiments, the lung cancer is squamous cell lung cancer or non-cell small lung cancer. In some embodiments, the pancreatic cancer is ductal carcinoma. In some embodiments, the lung cancer is squamous cell lung cancer.

In some embodiments, provided herein are methods for killing a cell comprising contacting the cell with a radiopharmaceutical conjugate or a pharmaceutically acceptable salt or stereoisomer thereof. In some embodiments, the cell expresses a CA-IX receptor. In some embodiments, the radiopharmaceutical conjugate or a pharmaceutically acceptable salt or stereoisomer thereof releases a number of alpha particles by natural radioactive decay. In some embodiments, the radiopharmaceutical conjugate or a pharmaceutically acceptable salt or stereoisomer thereof releases a number of beta particles, gamma rays, and/or Auger electrons by natural radioactive decay. The radiopharmaceutical conjugate described herein can kill a cell by radiation. In some embodiments, the radiopharmaceutical conjugate kills the cell directly by radiation. In some embodiments, the radiopharmaceutical conjugate kills the cell by inducing double-stranded DNA breaks. In some embodiments, the cell is a cancer cell. In some embodiments, the method comprises killing a cell with an alpha-particle emitting radionuclide. After contacting a cell, the radiopharmaceutical conjugate described herein can be internalized by the cell. The internalization can be mediated by cell receptors, cell membrane endocytosis, etc. In some embodiments, the radiopharmaceutical conjugate or a pharmaceutically acceptable salt or stereoisomer thereof is configured to treat cancer by ablating tumor cells.

In addition to the methods of treatment described above, the radiopharmaceutical conjugates and compositions described herein can be used to image, and/or as part of a treatment for diseases. Conjugates for imaging applications, e.g., single-photon emission computed tomography (SPECT) and positron emission tomography (PET), can comprise a radionuclide suitable for use as imaging isotopes (e.g., Dx in Table 3) such as the isotopes in Table 3. Accordingly, the radiopharmaceutical conjugate can be administered as a companion diagnostic.

In one aspect, described herein is a method of treatments that comprises administering a first radiopharmaceutical conjugate and a second radiopharmaceutical conjugate. The first radiopharmaceutical conjugate can be used as companion diagnostics and the second radiopharmaceutical conjugate can be used for therapeutics. In some embodiments, the first radiopharmaceutical conjugate and the second radiopharmaceutical conjugate have the same structure except for the radionuclide. In some embodiments, the first radiopharmaceutical conjugate comprises a gamma particle emitting radionuclide. In some embodiments, the first radiopharmaceutical conjugate comprises a radionuclide marked Dx of Table 3. In some embodiments, the first radiopharmaceutical conjugate comprises a radionuclide selected from Lu-177, In-111, Ga-68, Cu-64, and Zr-89. In some embodiments, the second radiopharmaceutical conjugate comprises an alpha or beta-particle emitting radionuclide. In some embodiments, the second radiopharmaceutical conjugate comprises a radionuclide marked Tx of Table 3. In some embodiments, the second radiopharmaceutical conjugate comprises Ac-225. In some embodiments, the method comprises administering (i) a first radiopharmaceutical conjugate comprising a radionuclide configured for companion diagnostic (such as PET imaging) and (ii) a second radiopharmaceutical conjugate comprising a radionuclide selected from an alpha or beta-particle emitter, wherein the first and the second radiopharmaceutical conjugate have the same structure except for the radionuclide.

In one aspect, described herein is a method of diagnosing or imaging a cancer in a subject in need thereof, comprising administering to the subject a radiopharmaceutical conjugate or a pharmaceutical composition described herein.

In some embodiments, the subject is 1 to 100 years old. In some embodiments, the subject is 5 to 10, 5 to 15, 5 to 18, 5 to 25, 5 to 35, 5 to 45, 5 to 55, 5 to 65, 5 to 75, 10 to 15, 10 to 18, 10 to 25, 10 to 35, 10 to 45, 10 to 55, 10 to 65, 10 to 75, 15 to 18, 15 to 25, 15 to 35, 15 to 45, 15 to 55, 15 to 65, 15 to 75, 18 to 25, 18 to 35, 18 to 45, 18 to 55, 18 to 65, 18 to 75, 25 to 35, 25 to 45, 25 to 55, 25 to 65, 25 to 75, 35 to 45, 35 to 55, 35 to 65, 35 to 75, 45 to 55, 45 to 65, 45 to 75, 55 to 65, 55 to 75, or 65 to 75 years old. In some embodiments, the subject is at least 5, 10, 15, 18, 25, 35, 45, 55, or 65 years old. In some embodiments, the subject is at most 10, 15, 18, 25, 35, 45, 55, 65, or 75 years old.

### Dosing

In certain embodiments, the compositions containing the radiopharmaceutical conjugates described herein are administered for therapeutic treatments. In certain therapeutic applications, the radiopharmaceutical conjugates described herein are administered to a patient already suffering from a disease or disorder, in an amount sufficient to cure or at least partially arrest at least one of the symptoms of the disease or disorder. Amounts effective for this use depend on the severity and course of the disease or disorder, previous therapy, the patient's health status, weight, and response to the drugs, and the judgment of the treating physician. The amount of radiopharmaceutical conjugates described herein or a pharmaceutically acceptable salt or stereoisomer thereof and/or pharmaceutical compositions administered can be sufficient to deliver a therapeutically effective dose of the particular subject. In some embodiments, radiopharmaceutical conjugate dosages can be between about 0.1 μg and about 50 mg per kilogram of body weight. In some embodiments, the dose of the radiopharmaceutical conjugate or a pharmaceutically acceptable salt or stereoisomer thereof described herein for the described methods is about 0.001 mg to about 1000 mg per dose for the subject being treated.

In some cases, the elimination profile of the conjugate can be adjusted by a reversible binding between the conjugate and a plasma protein such as albumin. A suitable affinity between the conjugate and the plasma protein can utilize the plasma protein as a reservoir for the conjugates, attaching and preserving the conjugates at high concentration and releasing the conjugates at a lower concentration, thereby improving elimination profile. In some embodiments, a dissociation constant (Kd) between the conjugate and human serum albumin is at most 500 μM, as determined at room temperature in human serum condition. In some embodiments, the Kd is from about 0.1 nM to about 1000 μM. In some embodiments, the Kd is at most 100 μM. In some embodiments, the Kd is at most 15 μM. In some embodiments, the Kd is from about 1 nM to about 10 μM. In some embodiments, the Kd is from about 10 nM to about 10 μM. In some embodiments, the Kd is from about 50 nM to about 1 μM. In some embodiments, the Kd is from about 100 nM to about 10 μM.

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

### Pharmaceutical Compositions/Formulations

The compounds described herein are administered to a subject in need thereof, either alone or in combination with pharmaceutically acceptable carriers, excipients, or diluents, in a pharmaceutical composition, according to standard pharmaceutical practice. In some embodiments, the compounds described herein are administered to animals.

In another aspect, provided herein are pharmaceutical compositions comprising a compound described herein, or a pharmaceutically acceptable salt or stereoisomer thereof, and at least one pharmaceutically acceptable excipient. Pharmaceutical compositions are formulated in a conventional manner using one or more pharmaceutically acceptable excipients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

### EXAMPLES

### A: Synthesis of the Compounds

### General Information

¹H NMR spectra were recorded on either a Bruker Avance III 400 (400 MHz), or Bruker Avance 300 (400 MHz) spectrometer. Chemical shifts are reported in ppm with solvent resonance as the internal standard (CDCl₃: 7.27 ppm, DMSO-d₆: 2.50 ppm, CD₃OD: 3.31 ppm). Data are reported as follows: chemical shift, integration, multiplicity (s=singlet, d=doublet, t=triplet, q=quartet, p=pentet, m=multiplet), and coupling constants (Hz).

Liquid chromatography was performed using forced flow (flash chromatography) on silica gel (SiO₂, 1000 mesh) or by column chromatography (silica gel, 1000 mesh). Thin layer chromatography (TLC) was performed on 20-25 μm silica gel glass backed plates. Preparative TLC was performed on a 40-45 μm silica gel glass backed plates. Visualization was performed using ultraviolet light (254 nm), iodide, or KMnO₄ in water.

### A1. Synthesis of Intermediate 1

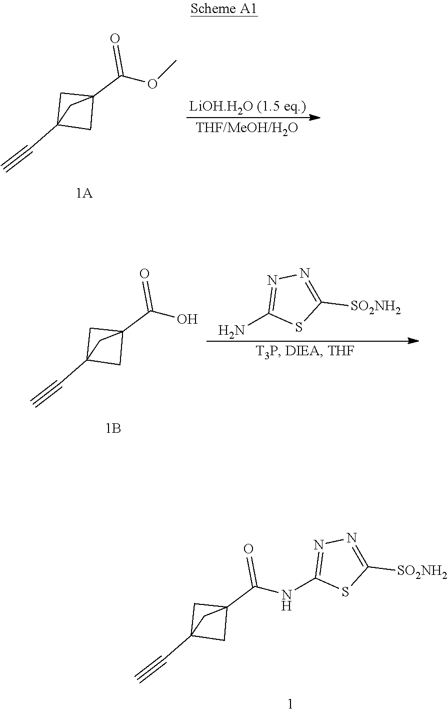

### 1.1 Preparation of Compound 1B

A solution of methyl 3-ethynylbicyclo[1.1.1]pentane-1-carboxylate (6.5 g, 43.3 mmol, 1 eq) and LiOH·H₂O (2.72 g, 64.9 mmol, 1.5 eq) in THF (20 mL), MeOH (20 mL) and H₂O (20 mL) was stirred at 20° C. for 1 h. The reaction mixture was concentrated under reduced pressure to remove organics and the aqueous phase adjusted to pH=5 by the addition of 1 M HCl. White solid precipitated and was filtered. The filter cake collected and dried under reduced pressure to give compound 1B (5.4 g, 92% yield) as a white solid; LCMS [M+H]: 137.1; ¹H NMR (400 MHz, DMSO-d₆) δ 12.51 (s, 1H), 3.12 (s, 1 H), 2.21 (s, 6H).

### 1.2 Preparation of Intermediate 1

To a solution of compound 1B (7.0 g, 51.4 mmol, 1 eq) and 5-amino-1,3,4-thiadiazole-2-sulfonamide (9.27 g, 51.4 mmol, 1 eq) in THF (40 mL) and DIEA (44.8 mL, 257 mmol, 5 eq) was added dropwise a solution of T³P (36.7 mL, 61.7 mmol, 50% w/w solution in EtOAc, 1.2 eq) in THF (4 mL) at 20° C. The mixture was stirred at 20° C. for 2 h. The mixture was diluted with H₂O (40 mL) and extracted with EtOAc (3×50 mL). The combined organic layers were washed with brine (20 mL), dried (Na₂SO₄), filtered and concentrated under reduced pressure. The crude product was triturated with H₂O (20 mL), filtered, and concentrated under reduced pressure to give Intermediate 1 (6.0 g, 39% yield) as a white solid; LCMS [M+H]: 299.0; ¹H NMR (400 MHz, DMSO-d₆) δ 13.17 (br s, 1H), 8.32 (s, 2H), 3.19 (s, 1H), 2.40 (s, 6H).

### A2. Synthesis of Intermediate 2

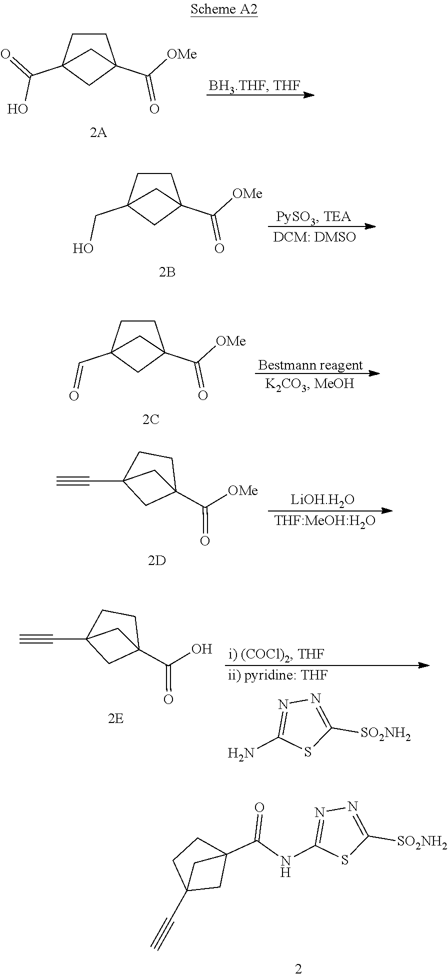

### 1.3. Preparation of Compound 2B

To a solution of compound 2A (3.5 g, 19.0 mmol, 1 eq) in THF (50 mL) was added 1M BH₃. THF (17.1 mL, 0.9 eq) at −20° C. The mixture was stirred at 0° C. for 0.5 h, quenched by addition of MeOH (20 mL) at 0° C. and concentrated under reduced pressure. The residue was purified by column chromatography (SiO₂, petroleum ether: EtOAc) to give compound 2B (2.0 g, 62% yield) as a white oil; ¹H NMR (400 MHz, CDCl₃) δ 3.71 (s, 2H), 3.68 (s, 3H), 1.99-1.94 (m, 2H), 1.80 (br d, J=1.4 Hz, 2H), 1.68-1.63 (m, 2H), 1.41 (dd, J=1.9, 4.1 Hz, 2H).

### 1.4. Preparation of Compound 2C

To a solution of compound 2B (3.1 g, 18.2 mmol, 1 eq) in DCM (30 mL) was added TEA (25.4 mL, 182 mmol, 10 eq) and a solution of PySO₃ (8.70 g, 54.6 mmol, 3 eq) in DMSO (10 mL). The mixture was stirred at 25° C. for 16 h. The residue was diluted with H₂O (50 mL) and extracted with DCM (3×30 mL). The combined organic layers were washed with brine (3×30 mL), dried (Na₂SO₄), filtered and concentrated under reduced pressure to give crude compound 2C (3.0 g, 98% yield) as a yellow oil which was used directly in the next reaction.

### 1.5. Preparation of Compound 2D

To a solution of compound 2C (3.0 g, 17.8 mmol, 1 eq) and Bestmann reagent (4.11 g, 21.4 mmol, 1.2 eq) in MeOH (30 mL) was added K₂CO₃ (4.93 g, 35.7 mmol, 2 eq) at 0° C. The mixture was stirred at 25° C. for 2 h. The reaction mixture was partitioned between MTBE (150 mL) and H₂O (300 mL). The organic phase was separated, washed with brine (3× 50 mL), dried (Na₂SO₄), filtered and concentrated under reduced pressure. The residue was purified by column chromatography (SiO₂, petroleum ether: EtOAc) to give compound 2D (1.7 g, 58% yield) as a white oil; ¹H NMR (400 MHz, CDCl₃) δ 3.62 (s, 3H), 2.19 (s, 1H), 2.01-1.97 (m, 2H), 1.87 (s, 4H), 1.62 (dd, J=2.0, 4.2 Hz, 2H).

### 1.6. Preparation of Compound 2E

To a solution of compound 2D (1.6 g, 9.74 mmol, 1 eq) in 2:2:1 THF:MeOH:H₂O (10 mL) was added LiOH·H₂O (818 mg, 19.5 mmol, 2 eq). The mixture was stirred at 25° C. for 4 h. The reaction mixture was concentrated under reduced pressure, the residue diluted with H₂O (8 mL) and acidified with 1M HCl (150 mL). White solids precipitated, the suspension filtered and the filtrate concentrated under reduced pressure to give compound 2E (1.6 g, 98% yield, 90% purity) as a white solid; ¹H NMR (400 MHz, CDCl₃) δ 2.24 (s, 1H), 2.02 (br s, 2H), 1.91 (s, 4H), 1.66 (dd, J=1.9, 4.1 Hz, 2H).

### 1.7. Preparation of Intermediate 2

To a solution of compound 2E (1.0 g, 6.7 mmol, 1 eq) in DCM (5 mL) was added oxalyl dichloride (991 μL, 11.3 mmol, 1.7 eq) at 0° C. The mixture was stirred at 25° C. for 1 h. The mixture was quenched with the addition of MeOH and concentrated under reduced pressure. The yellow oil was taken up in THF (10 mL) and added to a solution of 5-amino-1,3,4-thiadiazole-2-sulfonamide (1.23 g, 6.85 mmol) and pyridine (2.63 mL, 32.6 mmol) in THF (10 mL) at 0° C. The solution was stirred at 15° C. for 1 h and concentrated under reduced pressure. The residue was triturated with H₂O (120 ml) at 15° C. for 20 min to give Intermediate 2 (2.0 g, 98% yield) as a white solid; LCMS [M+H]: 313.0; ¹H NMR (400 MHz, DMSO-d₆) δ 12.97 (s, 1H), 8.33 (s, 2H), 3.21 (s, 1H), 2.11 (brd, J=1.3 Hz, 2H), 2.02-1.98 (m, 2H), 1.92-1.87 (m, 2H), 1.79 (dd, J=1.9, 4.0 Hz, 2H).

### A3. Synthesis of Intermediate 3

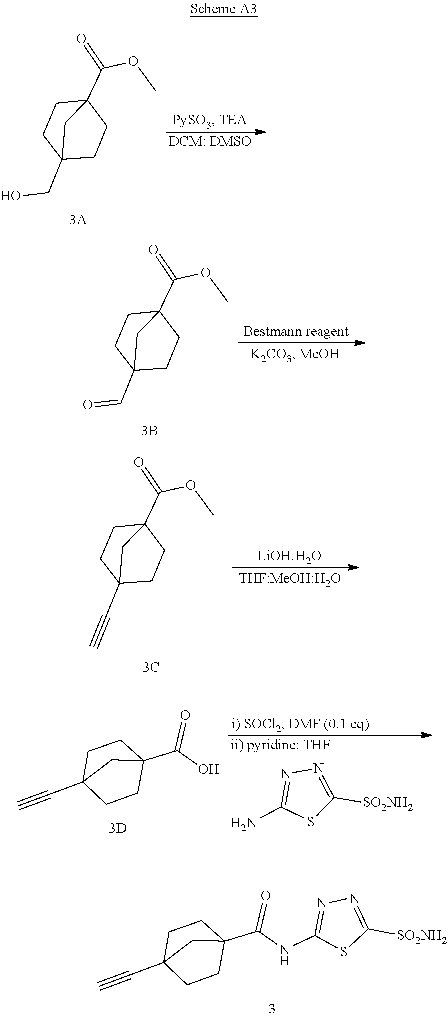

### 1.8 Preparation of Compound 3B

To a solution of compound 3A (9.0 g, 24.4 mmol, 1 eq) and TEA (68 mL, 244 mmol, 10 eq) in DCM (100 mL) was added a solution of PySO₃ (23.3 g, 73.3 mmol, 3 eq) in DMSO (40 mL) at 0° C. The mixture was stirred at 15° C. for 15 min then 30° C. for 16 h. The mixture was diluted with DCM (100 mL) and extracted with H₂O (10× 50 mL), dried (Na₂SO₄), filtered and concentrated under reduced pressure to give crude compound 3B (3.8 g) as colorless oil.

### 1.9 Preparation of Compound 3C

To a solution of compound 3B (8.5 g, 23.3 mmol, 1 eq) and Bestmann reagent (10.8 g, 28.0 mmol, 1.2 eq) in MeOH (90 mL) was added K₂CO₃ (12.9 g, 46.7 mmol, 2 eq) at 0° C. The mixture was stirred at 25° C. for 2 h. The mixture was diluted with MTBE (80 mL) and extracted with H₂O (3× 50 mL), dried (Na₂SO₄), filtered and concentrated under reduced pressure. The residue was purified by column chromatography (SiO₂, petroleum ether: EtOAc) to give compound 3C (7.3 g, 88% yield) as a white solid.

### 1.10 Preparation of Compound 3D

To a solution of compound 3C (7.3 g, 20.5 mmol, 1 eq) in 2:2:1THF:MeOH:H₂O (25 mL) was added LiOH·H₂O (2.58 g, 30.7 mmol, 1.5 eq). The mixture was stirred at 30° C. for 4 h and concentrated under reduced pressure. The residue was diluted with H₂O (8 mL) and acidified with 1M HCl (150 mL). White solids precipitated, the suspension filtered, and the filtrate concentrated under reduced pressure to give compound 3D (6.1 g, 91% yield) as a white solid.

### 1.11 Preparation of Intermediate 3

To a solution of compound 3D (6.10 g, 18.6 mmol, 1 eq) in 2-MeTHF (30 mL) was added DMF (286 μL, 1.86 mmol, 0.1 eq) and SOCl₂ (5.38 mL, 37.2 mmol, 2 eq) at 0° C. The mixture was stirred at 25° C. for 1 h and concentrated under reduced pressure. The residue in DMF (20 mL) was added dropwise to a solution of 5-amino-1,3,4-thiadiazole-2-sulfonamide (8.04 g, 44.6 mmol, 1.2 eq) and pyridine (9.0 mL, 112 mmol, 3 eq) in DMF (20 mL) at 0° C. The solution was stirred at 15° C. for 1 h and the reaction mixture concentrated under reduced pressure. The residue was triturated with H₂O (120 ml) at 15° C. for 20 min to give Intermediate 3 (10.3 g, 85% yield) as a white solid; LCMS [M+H]: 327.0; ¹H NMR (400 MHz, DMSO-d₆) δ 12.83 (s, 1H), 8.32 (s, 2H), 3.08 (d, J=1.1 Hz, 1H), 2.03-1.94 (m, 4H), 1.88-1.66 (m, 6H).

### A4. Synthesis of Intermediate 4

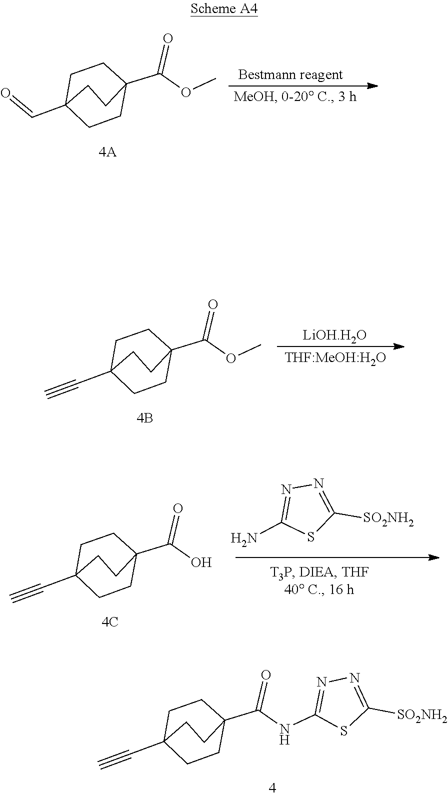

### 1.12 Preparation of Compound 4B

To a mixture of compound 4A (2.0 g, 10.2 mmol, 1 eq) and Bestmann reagent (2.35 g, 12.2 mmol, 1.2 eq) in MeOH (50 mL) and K₂CO₃ (2.82 g, 20.4 mmol, 2 eq) was added at 0° C. The mixture was stirred at 0° C. for 2 h, and then stirred at 20° C. for 1 h. The reaction mixture diluted with water (50 mL) and extracted with MTBE (3×50 mL). The combined organic layer was washed with brine (25 mL), dried (Na₂SO₄), filtered and the filtrate concentrated under reduced pressure. The residue was purified by column chromatography (SiO₂, petroleum ether: EtOAc) to give compound 4B (1.82 g, 93% yield) as a white solid; ¹H NMR (400 MHz, CHLOROFORM-d) δ 3.64 (s, 3H), 2.09 (s, 1H), 1.80 (s, 12H).

### 1.13 Preparation of Compound 4C

To a solution of compound 4B (1.82 g, 9.47 mmol, 1 eq) in 1:1:1 THF:MeOH:H₂O (60 mL) was added LiOH·H₂O (1.39 g, 33.1 mmol, 3.5 eq). The mixture was stirred at 15° C. for 3 h. The reaction mixture was washed with 1M HCl (43 mL), filtered and concentrated under reduced pressure to give compound 4C (1.58 g, 94% yield) as a white solid; ¹H NMR (400 MHz, CHLOROFORM-d) δ 7.27 (s, 1H), 2.10 (s, 1H), 1.85-1.80 (m, 12H).

### 1.14 Preparation of Intermediate 4

T³P (2.50 mL, 4.21 mmol, 50% purity, 1.5 eq) was added dropwise at 0° C. to a mixture of compound 4C (500 mg, 2.81 mmol, 1 eq), 5-amino-1,3,4-thiadiazole-2-sulfonamide (607 mg, 3.37 mmol, 1.2 eq) and DIEA (1.47 mL, 8.42 mmol, 3 eq) in THF (10 mL). The mixture was stirred at 40° C. for 16 h, diluted with water (20 mL) and extracted with EtOAc (3×20 mL). The combined organic layers were washed with brine (10 mL), dried (Na₂SO₄), filtered, and concentrated under reduced pressure. The crude product was triturated with EtOAc at 15° C. for 5 min to give Intermediate 4 (131 mg, 14% yield) as a white solid; LCMS [M+H]: 341.1.

### A5. Synthesis of Intermediate 5

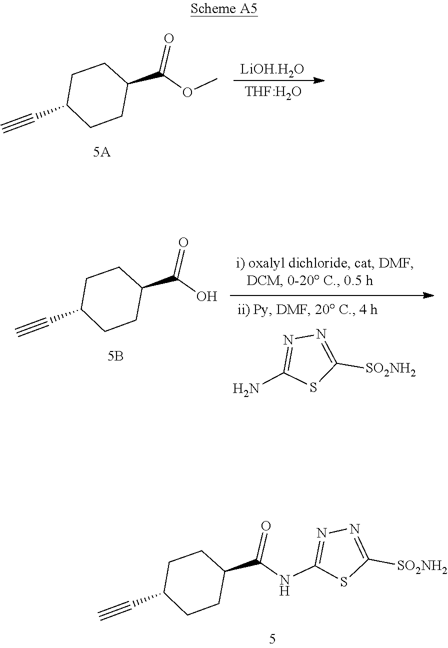

### 1.15 Preparation of Compound 5B

To a solution of compound 5A (2.0 g, 12.0 mmol, 1 eq) in THF (10 mL) and H₂O (10 mL) was added LiOH·H₂O (656 mg, 15.6 mmol, 1.3 eq). The mixture was stirred at 20° C. for 2 h and concentrated under reduced pressure. The residue was diluted with H₂O (8 mL) and acidified with 1 M HCl (30 mL). The white precipitate was filtered, and the filtrate concentrated under reduced pressure. The residue was recrystallization from H₂O to give compound 5B (1.72 g, 94% yield) as a white solid; LCMS [M+H]: 153.1; ¹H NMR (400 MHz, METHANOL-d₄) δ ppm 2.31 (d, J=2.32 Hz, 1H) 2.26 (br d, J=3.42 Hz, 2H), 1.98 (br d, J=9.78 Hz, 4H), 1.32-1.50 (m, 5H).

### 1.16 Preparation of Intermediate 5

To a solution of compound 5B (1.52 g, 9.99 mmol, 1 eq) in DCM (5 mL) was added DMF (77 μL, 999 μmol, 0.1 eq) and oxalyl chloride (870 μL, 9.99 mmol, 1 eq) at 0° C. The reaction mixture was stirred at 20° C. for 30 min and concentrated under reduced pressure. The oil was taken up in DMF (5 mL) and added to a solution of 5-amino-1,3,4-thiadiazole-2-sulfonamide (1.80 g, 9.96 mmol, 1 eq) and pyridine (804 μL, 9.96 mmol, 1 eq) in DMF (3 mL) at 0° C. The solution was stirred at 20° C. for 4 h, diluted with EtOAc (100 mL) and extracted with H₂O (3×50 mL). The combined organic layers were washed with brine (2×30 mL), dried (Na₂SO₄), filtered and concentrated under reduced pressure. The residue was further purified by recrystallization from H₂O (10 mL) to give Intermediate 5 (3.03 g, 97% yield) as a white solid; LCMS [M+H]: 315.1; ¹H NMR (400 MHz, DMSO-d₆) δ ppm 12.98 (s, 1H), 8.31 (s, 2H), 2.73 (s, 1H), 2.31 (br dd, J=12.9, 2.0 Hz, 2H), 1.84-2.02 (m, 4H), 1.23-1.52 (m, 4H).

### A6. Synthesis of Intermediate 6

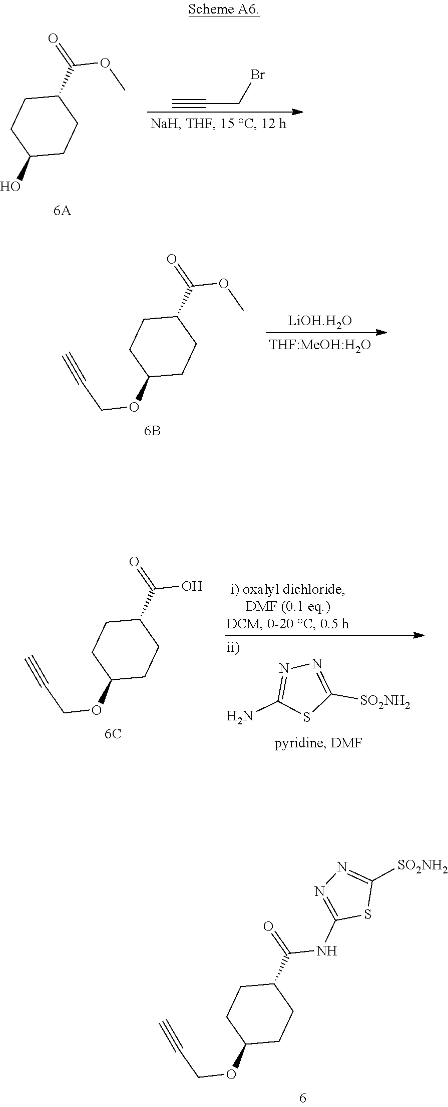

### 1.17 Preparation of Compound 6B

To a solution of compound 6A (12.5 g, 79.0 mmol, 1 eq) in THF (250 mL) was added NaH (3.79 g, 94.8 mmol, 60% purity, 1.2 eq) at 0° C. under a nitrogen atmosphere. 3-Bromoprop-1-yne (17 mL, 158 mmol, 80% purity, 2 eq) was added dropwise and the reaction stirred at 15° C. for 12 h. The mixture was purified by column chromatography (SiO₂, petroleum ether: EtOAc) to give compound 6B (2.5 g, 8.1% yield) as a yellow oil.

### 1.18 Preparation of Compound 6C

To a solution of compound 6B (2.5 g, 12.7 mmol, 1.0 eq) in 1:1:1 THF:MeOH:H₂O (15 mL) was added LiOH·H₂O (535 mg, 12.74 mmol, 1 eq) at 15° C. under a nitrogen atmosphere. The mixture was stirred at 15° C. for 12 h. The reaction was concentrated under reduced pressure. The residue was adjusted pH to 5-6 with 1M HCl (15 mL) and filtered. The filter cake was dried under vacuum to give compound 6C (1.4 g, 30% yield) as yellow solid; ¹H NMR (400 MHz, CHLOROFORM-d) δ 4.20 (d, 2H), 3.56-3.43 (m, 1H), 2.42 (t, J=2.3 Hz, 1H), 2.37-2.28 (m, 1H), 2.16-2.02 (m, 4H), 1.57-1.48 (m, 2H), 1.37-1.32 (m, 2H).

### 1.19 Preparation of Intermediate 6

Oxalyl chloride (576 μL, 6.59 mmol, 1 eq) was added dropwise to a solution of compound 6C (1.2 g, 6.59 mmol, 1 eq) and DMF (51 μL, 659 μmol, 0.1 eq) in DCM (12 mL) at 0° C. The mixture was stirred at 20° C. for 0.5 h and concentrated under reduced pressure. The yellow oil was taken up in DCM (8 mL) and added to a solution of 5-amino-1,3,4-thiadiazole-2-sulfonamide (1.17 g, 6.48 mmol, 1 eq) and pyridine (523 μL, 6.48 mmol, 1 eq) in DMF (8 mL) at 0° C. The mixture was stirred at 20° C. for 1 h, poured into H₂O (50 mL), stirred at 20° C. for 10 min, filtered and the filter cake dried under reduced pressure. The crude product was triturated with 1:1 petroleum ether:MTBE (20 mL) at 20° C. for 20 min to give Intermediate 6 (1.3 g, 51% yield, 88% purity) as a yellow solid; ¹H NMR (400 MHz, METHANOL-d₄) δ 4.21 (d, J=2.5 Hz, 2H) 3.54 (tt, J=10.7, 4.2 Hz, 1H) 2.82 (t, J=2.4 Hz, 1H) 2.5 (tt, J=11.8, 3.6 Hz, 1H) 2.12-2.23 (m, 2H) 1.96-2.07 (m, 2H) 1.54-1.69 (m, 2H) 1.25-1.38 (m, 2H).

### A7. Synthesis of Intermediate 7

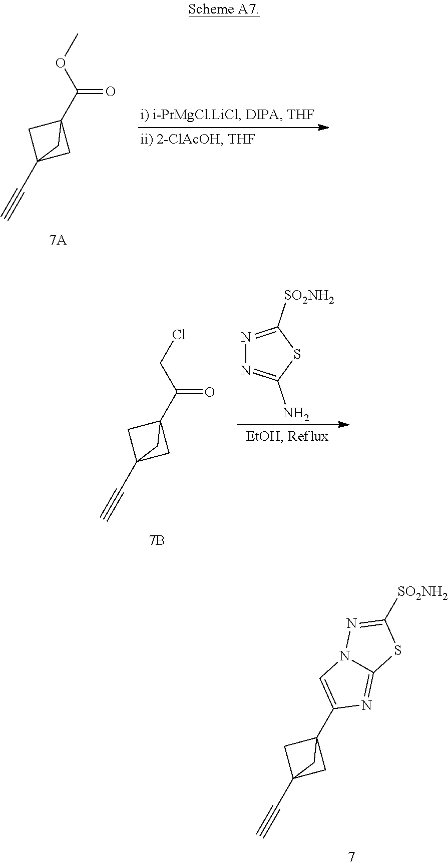

### 1.20 Preparation of Compound 7B

1.3 Mi-PrMgCl·LiCl (18.4 mL, 9 eq) was added dropwise to DIPA (3.76 mL, 26.6 mmol, 10 eq) and the mixture stirred at 10° C. for 3 h. A solution of compound 7A (400 mg, 2.66 mmol, 1 eq) and 2-chloroacetic acid (899 μL, 7.99 mmol, 3 eq) in THF (9 mL) was added to the reaction mixture at 0° C. The reaction stirred at 0° C. for 0.5 h and then warmed to 10° C. for 1 h. The reaction mixture was quenched by addition of 1M HCl (30 mL) at 0° C. and extracted with EtOAc (3×15 mL). The combined organic layers were washed with brine, dried (Na₂SO₄), filtered and concentrated under reduced pressure. The residue was purified by column chromatography (SiO₂, Petroleum ether:EtOAc) to give compound 7B (372 mg, 83% yield) as a white solid; ¹H NMR (400 MHz, METHANOL-d4) δ 4.44-4.35 (m, 2H), 2.51 (s, 1H), 2.34 (s, 6H).

### 1.21 Preparation of Intermediate 7

A mixture of compound 7B (280 mg, 1.66 mmol, 1 eq) and 5-amino-1,3,4-thiadiazole-2-sulfonamide (299 mg, 1.66 mmol, 1 eq) was heated in EtOH (4 mL) at 80° C. for 24 h under a nitrogen atmosphere. The reaction mixture was filtered and concentrated under reduced pressure. The residue was triturated with EtOAc at 10° C. for 10 min to give a white solid. The solid was dissolved in MeOH (3 mL) and 1 M NaHCO₃ (995 μL) was added. The mixture was stirred at 65° C. for 0.5 h under a nitrogen atmosphere, the reaction mixture cooled to room temperature, filtered and the filtrate concentrated under reduced pressure. The residue was triturated with H₂O at 15° C. for 10 min to give Intermediate 7 (183 mg, 62% yield) as a white solid; ¹H NMR (400 MHz, METHANOL-d4) δ 7.88 (s, 1H), 2.48 (s, 1H), 2.35 (s, 6H).

Intermediates in table 7.1 were prepared by procedures depicted in scheme A⁷.

TABLE 7.1

| Intermediate | Structure | m/z |
|---|---|---|
| 7.1 | 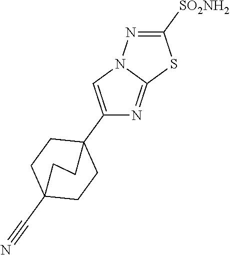 [SMILES: N#CC12CCC(c3cn4nc(S(N)(=O)=O)sc4n3)(CC1)CC2] | [M + H]: 337.1 |

### A8. Synthesis of Intermediate 8

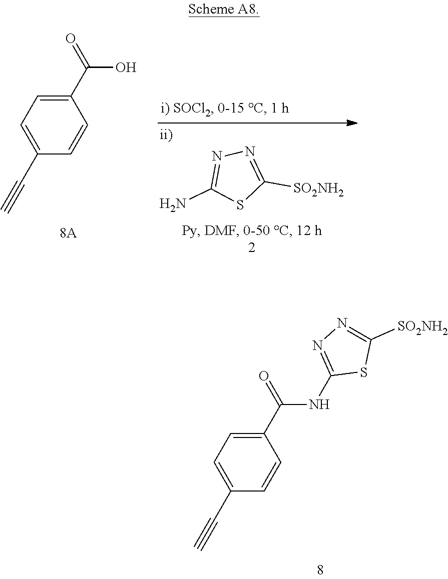 [SMILES: C#Cc1ccc(C(=O)O)cc1 ; Nc1nnc(S(N)(=O)=O)s1 ; C#Cc1ccc(C(=O)Nc2nnc(S(N)(=O)=O)s2)cc1]

### 1.22 Preparation of Intermediate 8

Compound 8A (2.0 g, 13.7 mmol, 1 eq) was added to SOCl₂ (20 mL) at 0° C. under a nitrogen atmosphere and the mixture stirred at 15° C. for 1 h. The reaction mixture was concentrated under reduced pressure and taken up in DMF (20 mL):pyridine (2.21 mL, 27.3 mmol, 2 eq). 5-Amino-1,3,4-thiadiazole-2-sulfonamide (2.25 g, 13.7 mmol, 1 eq) was added at 0° C. and the mixture stirred at 50° C. for 12 h. The crude product was triturated with H₂O at 15° C. for 10 min and triturated with EtOAc at 15° C. for 10 min to give crude Intermediate 8 (4.2 g) as a yellow solid; LCMS [M+H]: 309.0; ¹H NMR (400 MHz, DMSO-d₆) δ 13.85-13.34 (m, 1H), 8.38 (s, 2H), 8.14 (br d, J=8.3 Hz, 2H), 7.68 (br d, J=8.1 Hz, 2H), 4.59-4.42 (m, 1H).

Intermediates in table 6.1 were prepared by procedures depicted in scheme A⁸.

TABLE 6.1

| Intermediate | Structure | m/z |
|---|---|---|
| 8.1 | 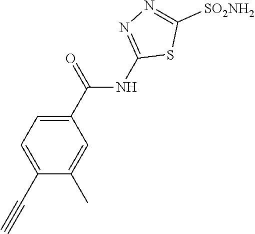 [SMILES: C#Cc1ccc(C(=O)Nc2nnc(S(N)(=O)=O)s2)cc1C] | [M + H]: 323.2 |
| 8.2 | 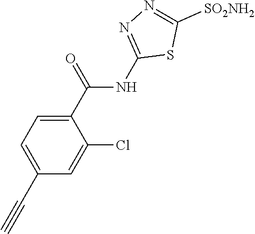 [SMILES: C#Cc1ccc(C(=O)Nc2nnc(S(N)(=O)=O)s2)c(Cl)c1] | [M + H]: 342.9/344.9 |

### A9. Synthesis of Intermediate 9

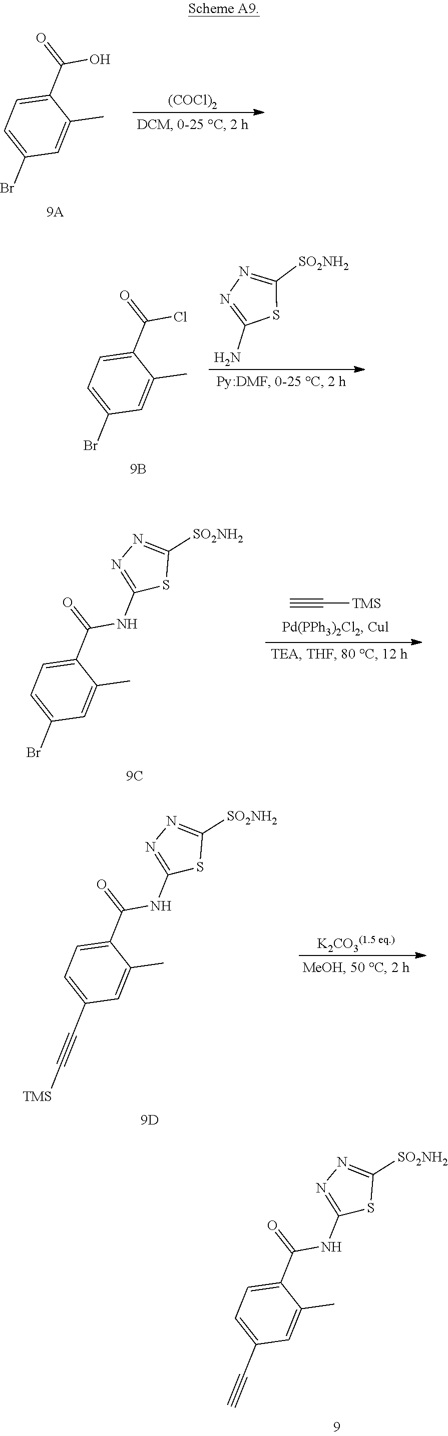

### 1.23 Preparation of Compound 9B

To a solution of compound 9A (2.50 g, 11.6 mmol, 1 eq) in DCM (50 mL) was added (COCl)₂ (5.09 mL, 58.1 mmol, 5 eq) and DMF (1.5 mL) at 0° C. and the mixture was stirred at 25° C. for 2 h. The reaction was repeated. The two reactions were combined and concentrated under reduced pressure to give crude compound 9B (5.4 g) as a yellow oil that was used directly in the next reaction.

### 1.24 Preparation of Compound 9C

To a solution of 5-amino-1,3,4-thiadiazole-2-sulfonamide (2.97 g, 16.5 mmol, 1 eq) in DMF (54 mL) was added pyridine (1.33 mL, 16.5 mmol, 1 eq). A solution of compound 9B (2.69 g, 11.5 mmol, 0.7 eq) in DMF (10 mL) was added at 0° C. and the mixture stirred at 25° C. for 2 h. The reaction was repeated and combined. The reaction mixture was poured into H₂O (100 mL) and extracted with EtOAc (3×50 mL). The combined organic layers were washed with brine (100 mL), dried (Na₂SO₄), filtered and concentrated under reduced pressure. The residue was purified by prep-HPLC (column: Phenomenex luna C18 (250*70 mm, 15 μm); mobile phase: [H₂O (0.1% TFA)-ACN]; gradient: 24%-54% B over 28.0 min) to give compound 9C (3.0 g, 24% yield) as white solid; LCMS [M+2H]: 379.0.

### 1.25 Preparation of Compound 9D

To a solution of compound 9C (1.2 g, 3.18 mmol, 1 eq) in THF (24 mL) was added Pd(PPh₃)₂Cl₂ (223 mg, 318 μmol, 0.1 eq), CuI (121 mg, 636 μmol, 0.2 eq), TEA (2.53 mL, 18.2 mmol) and ethyl(trimethyl) silane (1.32 mL, 9.54 mmol). The mixture stirred at 80° C. for 12 h. The reaction mixture was concentrated under reduced pressure and the residue purified by column chromatography (SiO₂, Petroleum ether:EtOAc) to give compound 9D (2.4 g, 83% yield, 87% purity) as yellow solid; LCMS [M+H]: 395.2.

### 1.26 Preparation of Intermediate 9

To a solution of compound 9D (2.0 g, 5.07 mmol, 1 eq) in MeOH (40 mL) was added K₂CO₃ (1.05 g, 7.60 mmol, 1.5 eq) at 50° C., and the mixture was stirred at 50° C. for 2 h. The mixture was adjusted to pH=3 with 1M HCl, the solid filtered and the filter cake concentrated to give intermediate 9 (1.4 g, 86% yield) as a yellow solid that was used directly in the next reaction; LCMS [M+H]: 323.1; ¹H NMR (400 MHz, DMSO-d6) δ 13.52 (br s, 1H), 8.38 (s, 2H), 7.66 (d, J=8.0 Hz, 1H), 7.52-7.40 (m, 2H), 4.40 (s, 1H), 2.40 (s, 3H).

Intermediates in table 6.2 were prepared by procedures depicted in scheme A9.

TABLE 6.2

| Intermediate | Structure | m/z |
|---|---|---|
| 9.1 | 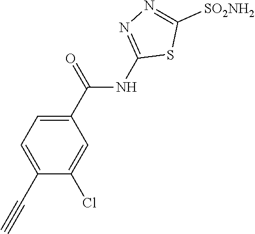 [SMILES: C#Cc1ccc(C(=O)Nc2nnc(S(N)(=O)=O)s2)cc1Cl] | [M + H]: 343.2/345.1 |

### A10. Synthesis of Intermediate 10

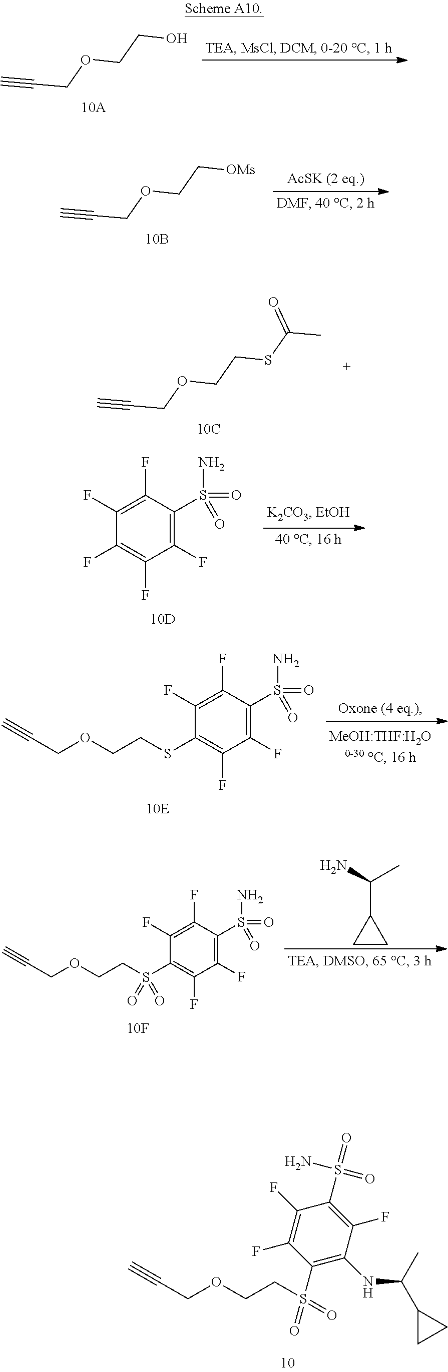

### 1.27 Preparation of Compound 10B

To a solution of compound 10A (10 g, 99.9 mmol, 1 eq) in DCM (100 mL) were added TEA (27.8 mL, 200 mmol, 2 eq) and MsCl (11.6 mL, 150 mmol, 1.5 eq) at 0° C. under a nitrogen atmosphere. The mixture was stirred at 20° C. for 1 h. The reaction mixture was washed with NaHCO₃ (50 mL), NH₄Cl (50 ml), H₂O (20 ml) and brine (20 mL). The organic layer was dried (Na₂SO₄), filtered, and concentrated under reduced pressure to give compound 10B (17.8 g, 100% yield) as a yellow oil; ¹H NMR (400 MHz, CHLOROFORM-d) δ 4.44-4.36 (m, 2H), 4.25-4.17 (m, 2H), 3.85-3.73 (m, 2H), 3.10-3.03 (m, 3H), 2.52-2.42 (m, 1H).

### 1.28 Preparation of Compound 10C

To a solution of compound 10B (17.8 g, 99.9 mmol, 1 eq) in DMF (200 mL) was added potassium ethanethioate (22.8 g, 200 mmol, 2 eq) at 20° C. under a nitrogen atmosphere. The mixture was stirred at 40° C. for 2 h. The residue was dissolved in EtOAc (500 mL), washed with H₂O (2×100 mL) and brine (2×100 mL). The organic phase was dried (Na₂SO₄), filtered, and concentrated under reduced pressure. The residue was purified by column chromatography (SiO₂, petroleum ether:EtOAc) to give compound 10C (9.2 g, 58% yield) as a light yellow liquid; ¹H NMR (400 MHz, CHLOROFORM-d) δ 4.16 (d, J=2.4 Hz, 2H), 3.66 (t, J=6.4 Hz, 2H), 3.11 (t, J=6.4 Hz, 2H), 2.44 (t, J=2.4 Hz, 1H), 2.34 (s, 3H).

### 1.29 Preparation of Compound 10E

To a solution of compound 10C (7.11 g, 44.9 mmol, 1 eq) and compound 10D (11.1 g, 44.9 mmol, 1 eq) in EtOH (100 mL) was added K₂CO₃ (31.1 g, 225 mmol, 5 eq) at 20° C. The mixture was stirred at 40° C. for 16 h. The reaction mixture was diluted with H₂O (160 mL) and extracted with EtOAc (3× 400 mL). The combined organic layer was dried (Na₂SO₄), filtered and concentrated under reduced pressure. The residue was purified by column chromatography (SiO₂, Petroleum ether:EtOAc) to give compound 10E (6.4 g, 41% yield) as a white solid; LCMS [M+H]: 344.0. ¹H NMR (400 MHz, DMSO-d₆) δ 8.39 (s, 2H), 4.10 (d, J=2.4 Hz, 2H), 3.64 (t, J=5.8 Hz, 2H), 3.38 (t, J=2.4 Hz, 1H), 3.24 (t, J=5.8 Hz, 2H).

### 1.30 Preparation of Compound 10F

To a solution of compound 10E (2.0 g, 5.83 mmol, 1 eq) in MeOH (24 mL) and THF (8 mL) was added Oxone (14.3 g, 23.3 mmol, 4 eq) in H₂O (16 mL) at 0° C. The mixture was stirred at 30° C. for 16 h. The reaction mixture was diluted with H₂O (30 mL) and extracted with DCM (2×50 mL). The combined organic layer was dried (Na₂SO₄), filtered and concentrated under reduced pressure. The residue was purified by column chromatography (SiO₂, Petroleum ether:EtOAc) to give compound 10F (1.4 g, 64% yield) as a white solid; LCMS [M−H]: 373.9; ¹H NMR (400 MHz, DMSO-d₆) δ 8.61 (s, 2H), 4.02 (d, J=2.4 Hz, 2H), 3.96-3.90 (m, 2H), 3.89-3.84 (m, 2H), 3.36 (t, J=2.4 Hz, 1H).

### 1.31 Preparation of Intermediate 10

To a solution of compound 10F (1.2 g, 3.20 mmol, 1 eq) in DMSO (12 mL) was added TEA (890 μL, 6.39 mmol, 2 eq) and(S)-1-cyclopropylethan-1-amine (408 mg, 4.80 mmol, 1.5 eq) under a nitrogen atmosphere. The mixture was stirred at 65° C. for 3 h. The residue was diluted with H₂O (30 mL) and extracted with DCM (3×30 mL). The combined organic layer was washed with brine (2×10 mL), dried (Na₂SO₄), filtered and concentrated under reduced pressure. The crude product was purified by column chromatography (SiO₂, petroleum ether:EtOAc) to give Intermediate 10 (840 mg, 60% yield) as a yellow oil; LCMS [M−H]: 439.3; ¹H NMR (400 MHz, DMSO-d₆) δ 8.32 (s, 2H), 6.51 (br d, J=8.6 Hz, 1H), 4.05 (d, J=2.4 Hz, 2H), 3.86 (d, J=1.8 Hz, 4H), 3.39 (t, J=2.4 Hz, 1H), 3.14 (td, J=6.4, 8.4 Hz, 1H), 1.29-1.08 (m, 5H), 0.97-0.78 (m, 1H), 0.51-0.35 (m, 2H), 0.28-0.14 (m, 2H).

Intermediates in table 6.3 were prepared by procedures depicted in Scheme A¹⁰.

TABLE 6.3

| Intermediate | Structure | m/z | ¹H NMR |
|---|---|---|---|
| 10.1 | 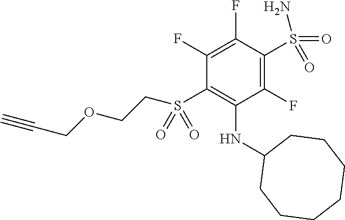 [SMILES: C#CCOCCS(=O)(=O)c1c(F)c(F)c(S(N)(=O)=O)c(F)c1NC1CCCCCCC1] | [M − H]^{:} 481.4 | ¹H NMR (400 MHz, DMSO-d₆) δ 8.31 (s, 2H), 6.60 (br d, J = 8.6 Hz, 1H), 4.05 (d, J = 2.4 Hz, 2H), 3.84 (s, 4H), 3.78 (br dd, J = 3.8, 7.8 Hz, 1H), 3.38 (t, J = 2.4 Hz, 1H), 1.92-1.79 (m, 2H), 1.74-1.43 (m, 12H) |
| 10.2 | 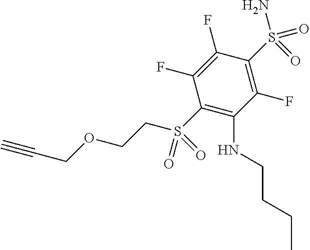 [SMILES: C#CCOCCS(=O)(=O)c1c(F)c(F)c(S(N)(=O)=O)c(F)c1NCCCC] | [M − H]^{:} 427.0 | ¹H NMR (400 MHz, DMSO- d6) δ 8.26 (br s, 2H), 6.56 (br t, J = 4.4 Hz, 1H), 4.08-4.01 (m, 2H), 3.90-3.79 (m, 3H), 3.38 (t, J = 2.4 Hz, 1H), 3.37-3.26 (m, 2H), 1.63-1.48 (m, 2H), 1.43-1.26 (m, 2H), 1.01-0.84 (m, 3H) |
| 10.3 | 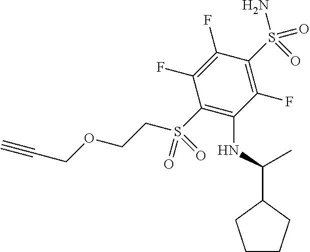 [SMILES: C#CCOCCS(=O)(=O)c1c(F)c(F)c(S(N)(=O)=O)c(F)c1N[C@@H](C)C1CCCC1] | [M − H]^{:} 467.4 | ¹H NMR (400 MHz, DMSO- d6) δ 8.31 (s, 2H), 6.53 (br d, J = 9.0 Hz, 1H), 4.07-4.03 (m, 2H), 3.84 (s, 4H), 3.65-3.54 (m, 1H), 3.38 (s, 1H), 1.95- 1.45 (m, 7H), 1.35-1.21 (m, 2H), 1.14-1.07 (m, 3H). |
| 10.4 | 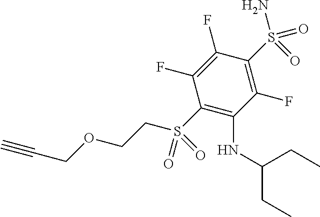 [SMILES: C#CCOCCS(=O)(=O)c1c(F)c(F)c(S(N)(=O)=O)c(F)c1NC(CC)CC] | [M − H]^{:} 441.3 | ¹H NMR (400 MHz, DMSO- d6) δ 8.31 (br s, 2H), 6.49 (br d, J = 9.0 Hz, 1H), 4.05 (d, J = 2.4 Hz, 2H), 3.85 (s, 4H), 3.61- 3.48 (m, 1H), 3.38 (t, J = 2.4 Hz, 1H), 1.63-1.38 (m, 4H), 0.97-0.82 (m, 6H). |

### A11. Synthesis of Intermediate 11

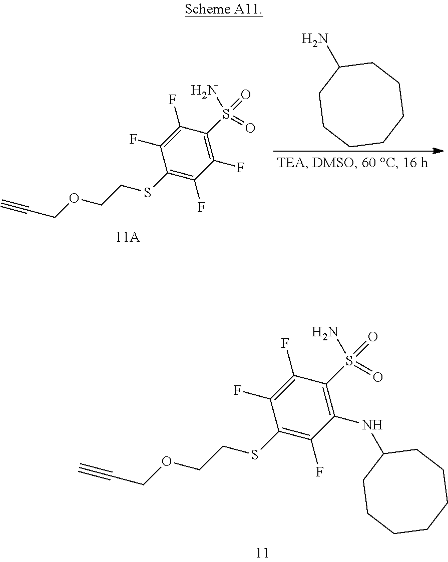 [SMILES: C#CCOCCSc1c(F)c(F)c(S(N)(=O)=O)c(F)c1F ; NC1CCCCCCC1 ; C#CCOCCSc1c(F)c(F)c(S(N)(=O)=O)c(NC2CCCCCCC2)c1F]

### 1.32 Preparation of Intermediate 11

To a solution of compound 11A (1.7 g, 4.95 mmol, 1 eq) in DMSO (17 mL) was added TEA (1.38 mL, 9.90 mmol, 2 eq) and cyclooctanamine (1.26 g, 9.90 mmol, 2 eq). The mixture was stirred at 60° C. for 16 h, diluted with H₂O (40 mL) and extracted with EtOAc (2×40 mL). The combined organic layer was dried (Na₂SO₄), filtered and concentrated under reduced pressure. The residue was purified by column chromatography (SiO₂, Petroleum ether: EtOAc) to give intermediate 11 (1.4 g, 63% yield) as a yellow oil; LCMS [M−H]: 449.4; ¹H NMR (400 MHz, DMSO-d₆) δ 8.10 (s, 2H), 6.38-6.25 (m, 1H), 4.11 (d, J=2.4 Hz, 2H), 3.71 (br dd, J=3.8, 7.8 Hz, 1H), 3.61 (t, J=6.2 Hz, 2H), 3.38 (t, J=2.4 Hz, 1H), 3.17 (t, J=6.2 Hz, 2H), 1.87-1.74 (m, 2H), 1.70-1.59 (m, 2H), 1.57-1.37 (m, 10H).

### A12. Synthesis of Intermediate 12

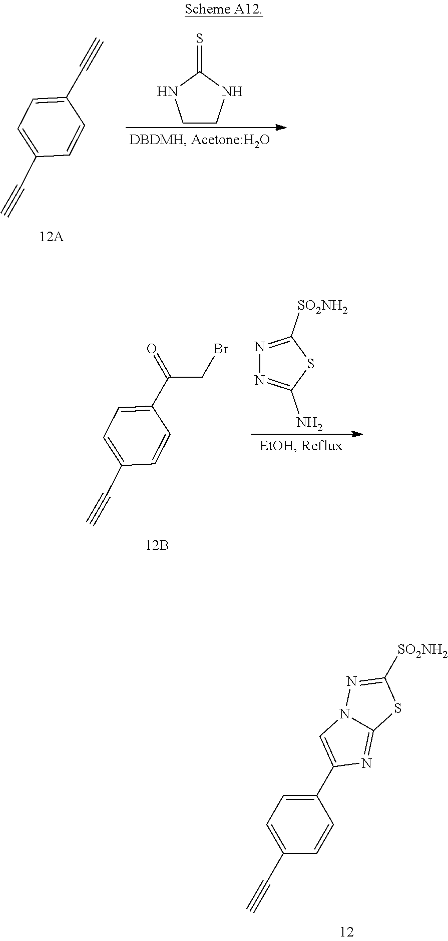

### 1.33 Preparation of Compound 12B

To a solution of compound 12A (4.0 g, 31.7 mmol, 1 eq) in acetone (160 mL) and H₂O (2.8 mL) was added DBDMH (9.07 g, 31.7 mmol, 1 eq) and imidazolidine-2-thione (486 mg, 4.76 mmol, 0.15 eq) at 25° C. The reaction was stirred at 45° C. for 12 h, filtered and the filtrate concentrated under reduced pressure. The residue was purified by silica gel chromatography (SiO₂, petroleum ether:EtOAc) to give compound 12B (3.8 g, 54% yield) as yellow solid; ¹H NMR (400 MHz, CDCl₃) δ ppm 7.96 (2H, d, J=8.44 Hz) 7.61 (2H, d, J=8.44 Hz) 4.44 (2 H, s) 3.30 (1 H, s).

### 1.34 Preparation of Intermediate 12

To a solution of compound 12B (1.9 g, 8.52 mmol, 1 eq) in EtOH (19 mL) was added 5-amino-1,3,4-thiadiazole-2-sulfonamide (1.53 g, 8.52 mmol, 1 eq) and the mixture stirred at 80° C. for 12 h. The procedure was repeated. The two batches were combined and filtered. The filter cake was collected and purified by prep-HPLC (column: Phenomenex Titank C18 Bulk 250*70 mm 10 u; mobile phase: [H₂O (0.1% TFA)-ACN]; gradient: 25%-65% B over 20.0 min) to give Intermediate 12 (2.0 g, 39% yield) as yellow solid; ¹H NMR (400 MHz, DMSO-d6) δ 8.96 (s, 1H), 8.75 (s, 2H), 7.97-7.88 (m, 2H), 7.55 (d, J=8.4 Hz, 2H), 4.25 (s, 1H).

### A13. Synthesis of Intermediate 13

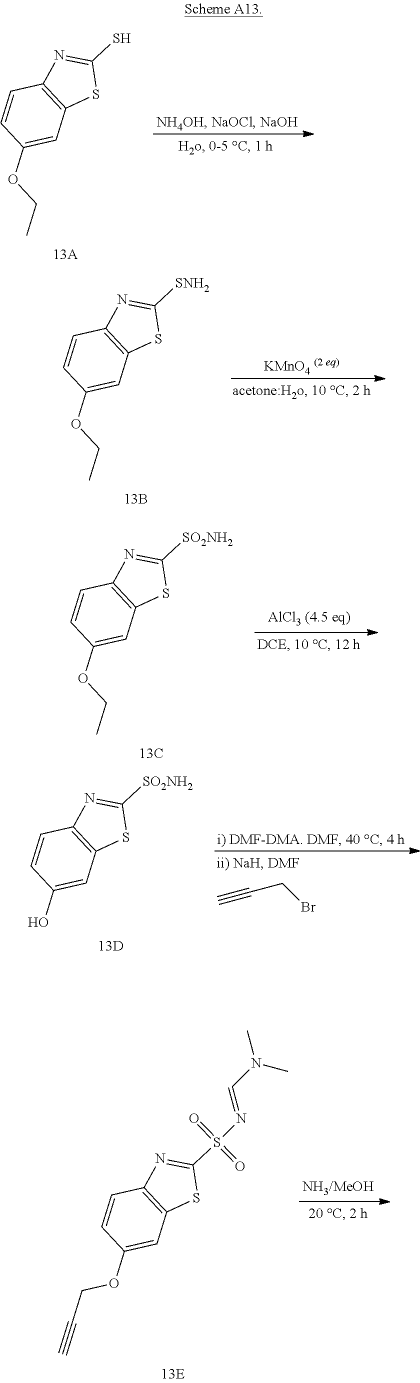

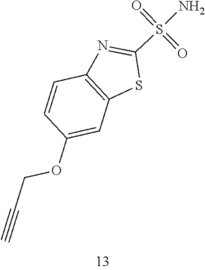 [SMILES: C#CCOc1ccc2nc(S(N)(=O)=O)sc2c1]

### 1.35 Preparation of Compound 13B

To a solution of compound 13A (2.0 g, 9.47 mmol, 1 eq) and NaOH (398 mg, 9.94 mmol, 1.05 eq) in H₂O (24 mL) was added NH₃·H₂O (7.24 mL, 52.6 mmol, 28% purity, 5.6 eq) dropwise. NaClO (3.44 mL, 2.93 mmol, 5.3% purity) was added dropwise at 0° C. and the resulting mixture stirred at 0-5° C. for 1 h. The mixture was filtered and the filter cake washed with H₂O (3 mL) to give crude compound 13B (2.78 g) as a white solid; LCMS [M+H]: 227.0; ¹H NMR (400 MHz, DMSO-d₆) δ 7.64-7.54 (m, 2H), 7.03-6.95 (m, 1H), 4.88 (s, 2H), 4.13-4.02 (m, 2H), 1.34 (t, J=7.0 Hz, 3H). 1.36 Preparation of compound 13C

To a solution of compound 13B (2.78 g, 12.3 mmol, 1 eq) in acetone (60 mL) and H₂O (60 mL) was added a solution of KMnO₄ (3.88 g, 24.6 mmol, 2 eq) in H₂O (90 mL) dropwise at 0° C. The reaction mixture was stirred at 10° C. for 2 h. The mixture was treated with activated carbon and 10 N NaOH (4 mL), stirred for 30 min, filtered, and washed with EtOAc (50 mL). To the filtrate was added H₂O (50 mL), the mixture extracted with EtOAc (3×50 mL), dried (Na₂SO₄) and concentrated under reduced pressure. The residue was purified by column chromatography (SiO₂, petroleum ether:EtOAc) to give compound 13C (2.1 g, 66% yield) as a white solid; LCMS [M+H]: 259.0; ¹H NMR (400 MHz, DMSO-d₆) δ 8.24 (s, 2H), 8.03 (d, J=9.1 Hz, 1H), 7.79 (d, J=2.5 Hz, 1H), 7.29-7.19 (m, 1H), 4.20-4.06 (m, 2H), 1.38 (t, J=7.0 Hz, 3H).

### 1.37 Preparation of Compound 13D

To a mixture of compound 13C (2.5 g, 9.68 mmol, 1 eq) in DCE (39 mL) was added AlCl₃ (2.38 mL, 43.6 mmol, 4.5 eq) and the mixture stirred at 25° C. for 12 h under a nitrogen atmosphere. The reaction was quenched with H₂O (6 mL) and concentrated under reduced pressure. The residue was purified by column chromatography (SiO₂, petroleum ether:EtOAc) to give compound 13D (2.13 g, 96% yield) as a white solid; LCMS [M+H]: 230.9; ¹H NMR (400 MHz, DMSO-d₆) δ 10.22 (s, 1H), 8.18 (s, 2H), 7.95 (d, J=8.9 Hz, 1H), 7.50 (d, J=2.4 Hz, 1H), 7.14-7.07 (m, 1H).

### 1.38 Preparation of Compound 13E

To a solution of compound 13D (0.5 g, 2.17 mmol, 1 eq) in DMF (5 mL) was added DMF-DMA (1.91 mL, 14.4 mmol) at 20° C. The mixture was stirred at 40° C. for 4 h. The mixture was concentrated, the residue dissolved in DMF (5 mL), NaH (130 mg, 3.26 mmol, 60% purity, 1.50 eq) added at 0° C. and the mixture stirred at 0° C. for 0.5 h. 3-Bromoprop-1-yne (373 μL, 3.46 mmol, 1.59 eq) in DMF (0.5 mL) was added at 0° C., the mixture stirred at 20° C. for 2 h and the residue poured into a mixture of water (5 mL) and EtOAc (5 mL). The mixture was filtered, the filter cake collected, dissolved in DCM (10 mL), and washed with saturated aqueous NaHCO₃ (3×8 mL). The residue was purified by column chromatography (SiO₂, DCM/MeOH) to give compound 13E (240 mg, 34% yield) as a brown solid; ¹H NMR (400 MHz, DMSO-d₆) δ 8.37 (s, 1H), 8.06 (br d, J=9.0 Hz, 1H), 7.74-7.88 (m, 1H), 7.28 (br dd, J=9.01, 2.00 Hz, 1H), 4.92 (br d, J=1.75 Hz, 2H), 3.63 (s, 1H), 3.24 (s, 3H), 2.99 (s, 3H).

### 1.39 Preparation of Intermediate 13

To a solution of compound 13E (0.24 g, 742 μmol, 1 eq) in NH₃/MeOH (10 mL) was stirred at 20° C. for 2 h. The mixture was adjusted to pH=2 with conc. HCl and concentrated under reduced pressure. The solid was triturated with ice-water (5 mL), filtered and the filter cake was concentrated to give Intermediate 13 (0.16 g, 80% yield) as a yellow solid; LCMS [M+H]: 269.0.

### A14. Synthesis of Intermediate 14

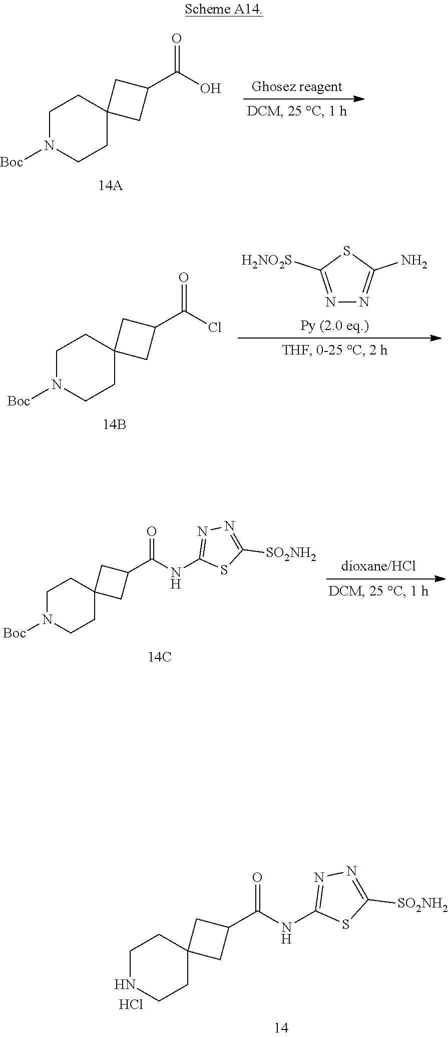 [SMILES: CC(C)(C)OC(=O)N1CCC2(CC1)CC(C(=O)O)C2 ; CC(C)(C)OC(=O)N1CCC2(CC1)CC(C(=O)Cl)C2 ; Nc1nnc(S(N)(=O)=O)s1 ; CC(C)(C)OC(=O)N1CCC2(CC1)CC(C(=O)Nc1nnc(S(N)(=O)=O)s1)C2 ; NS(=O)(=O)c1nnc(NC(=O)C2CC3(CCNCC3)C2)s1 ; Cl]

### 1.40 Preparation of Compound 14B

To a solution of compound 14A (20 g, 74.3 mmol, 1 eq) in DCM (300 mL) was added Ghosez reagent (11.8 mL, 89.1 mmol, 1.2 eq), and the mixture stirred at 25° C. for 1 h. The reaction mixture was concentrated under reduced pressure to give compound 14B (21 g, 98% yield) as a yellow oil that was used directly in the next reaction.

### 1.41 Preparation of Compound 14C

To a solution of 5-amino-1,3,4-thiadiazole-2-sulfonamide (12.5 g, 70 mmol, 1 eq) in THF (200 mL) was added pyridine (11.2 mL, 139 mmol, 2 eq). A solution of compound 14B (20 g, 69.5 mmol, 1 eq) in THF (20 mL) was added at 0° C. and the mixture stirred at 25° C. for 2 h. The aqueous layer was extracted with EtOAc (2×300 mL). The combined organic layers were washed with brine (300 mL), dried (Na₂SO₄), filtered and concentrated under reduced pressure. The residue was triturated with petroleum ether:EtOAc (200 mL), stirred for 5 min, filtered and the filter cake collected to give compound 14C (24 g, 80% yield) as a white solid; ¹H NMR (400 MHz, DMSO-d₆) δ 12.91 (s, 1H), 8.31 (s, 2H), 3.42-3.36 (m, 1H), 3.30-3.24 (m, 2H), 3.23-3.10 (m, 2H), 2.12-1.97 (m, 4H), 1.58-1.49 (m, 2H), 1.47-1.31 (m, 11H).

### 1.42 Preparation of Intermediate 14

To a solution of compound 14C (25 g, 57.9 mmol, 1 eq) in DCM (500 mL) was added 4 M HCl:dioxane (579 mL, 40 eq), and the mixture stirred at 25° C. for 1 h. The reaction mixture was concentrated under reduced pressure to give crude Intermediate 14 (26 g) as a white solid; LCMS [M−H]: 330.2; ¹H NMR (400 MHz, DMSO-d6) δ 13.02 (br s, 1H), 9.15-8.86 (m, 2H), 3.48-3.34 (m, 1H), 3.05-2.81 (m, 4H), 2.21-1.97 (m, 4H), 1.86-1.62 (m, 4H).

### A15. Synthesis of Intermediate 15

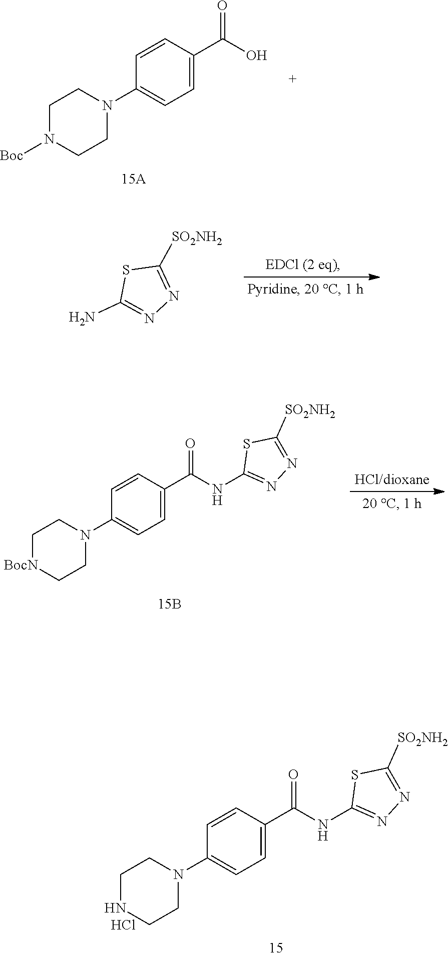 [SMILES: CC(C)(C)OC(=O)N1CCN(c2ccc(C(=O)O)cc2)CC1 ; Nc1nnc(S(N)(=O)=O)s1 ; CC(C)(C)OC(=O)N1CCN(c2ccc(C(=O)Nc3nnc(S(N)(=O)=O)s3)cc2)CC1 ; NS(=O)(=O)c1nnc(NC(=O)c2ccc(N3CCNCC3)cc2)s1 ; Cl]

1.43 Preparation of compound 15B

To a solution of compound 15A (3.0 g, 9.8 mmol, 1 eq) and 5-amino-1,3,4-thiadiazole-2-sulfonamide (3.53 g, 19.6 mmol, 2 eq) in pyridine (12 mL) was added EDCl (3.75 g, 19.6 mmol, 2 eq). The mixture was stirred at 20° C. for 1 h and concentrated under reduced pressure. The solid was washed with MeOH (3×30 mL), the mixture filtered and the filter cake dried under reduced pressure to give compound 15B as a yellow solid; LCMS [M+H]: 469.2; ¹H NMR (400 MHz, DMSO-d6) δ 13.14 (br s, 1H), 8.31 (s, 2H), 8.07 (brd, J=8.8 Hz, 2H), 7.04 (br d, J=8.8 Hz, 2H), 3.53-3.30 (m, 8H), 1.42 (s, 9H).

### 1.44 Preparation of Intermediate 15

A solution of compound 15B (2.8 g, 5.98 mmol, 1 eq) in HCl/dioxane (28 mL) was stirred at 20° C. for 1 h and concentrated under reduced pressure. The residue was triturated with MTBE to give Intermediate 15 as a white sold; LCMS [M+H]: 369.2; ¹H NMR (400 MHz, DMSO-d6) δ 13.36-13.04 (m, 1H), 9.49 (br s, 2H), 8.34 (s, 2H), 8.10 (br d, J=8.8 Hz, 2H), 7.11 (br d, J=9.0 Hz, 2H), 3.70-3.59 (m, 4H), 3.19 (br s, 4H).

### A16. Synthesis of Intermediate 16

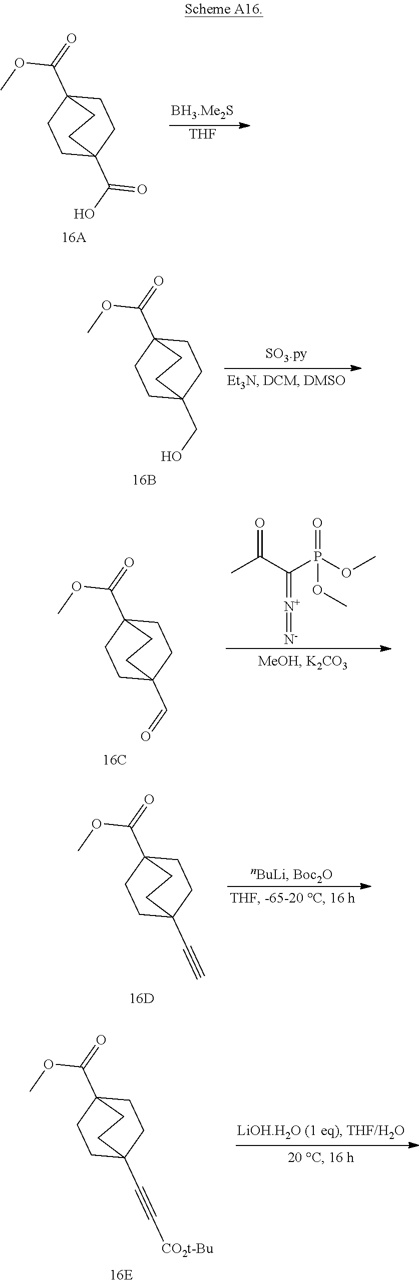

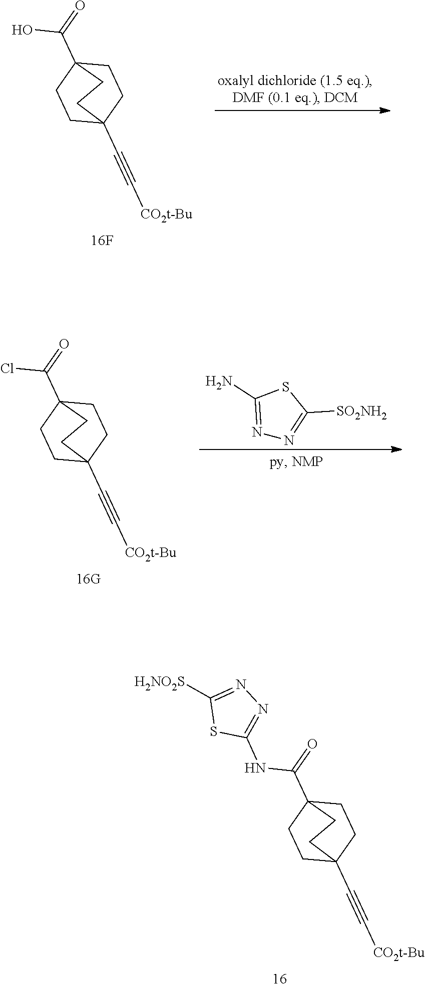 [SMILES: CC(C)(C)OC(=O)C#CC12CCC(C(=O)O)(CC1)CC2 ; CC(C)(C)OC(=O)C#CC12CCC(C(=O)Cl)(CC1)CC2 ; Nc1nnc(S(N)(=O)=O)s1 ; CC(C)(C)OC(=O)C#CC12CCC(C(=O)Nc3nnc(S(N)(=O)=O)s3)(CC1)CC2]

### 1.45 Preparation of Compound 16B

Compound 16A (20 g, 94.2 mmol, 1 eq) was taken up in THF (200 mL). The mixture degassed and purged with nitrogen 3 times, cooled to 0° C., 10 M borane methylsulfanylmethane (23.6 mL, 2.5 eq) added dropwise for 15 minutes. The reaction was stirred at 20° C. for 2 h. The reaction mixture was quenched by addition of MeOH (200 mL) at 20° C. and concentrated under reduced pressure to give crude Compound 16B (18.7 g) as a colorless oil; ¹H NMR (400 MHz, DMSO-d₆) δ 4.35 (t, J=5.6 Hz, 1H), 3.55 (s, 3H), 3.02 (d, J=5.4 Hz, 2H), 1.75-1.57 (m, 6H), 1.42-1.27 (m, 6H).

### 1.46 Preparation of Compound 16C

To a solution of compound 16B (18.7 g, 94.2 mmol, 1 eq) in DCM (160 mL) was added TEA (131 mL, 942 mmol, 10 eq) at 20° C. Pyridine;sulfur trioxide (45 g, 283 mmol, 3 eq) was dissolved in DMSO (100 mL), stirred for 15 minutes at 20° C. then added to the reaction mixture at 0° C. The mixture was stirred at 30° C. for 16 h, diluted with DCM (200 mL). The combined organic layers were washed with H₂O (3×200 mL) and brine (2×200 mL), dried (Na₂SO₄), filtered and concentrated under reduced pressure to give crude compound 16C (18.5 g) as a yellow oil; ¹H NMR (400 MHz, DMSO-d₆) δ 9.41 (s, 1H), 3.58 (s, 3H), 1.80-1.68 (m, 6H), 1.64-1.54 (m, 6H).

### 1.47 Preparation of Compound 16D

To a solution of compound 16C (18.5 g, 94.2 mmol, 1 eq) in MeOH (185 mL) was added K₂CO₃ (26.0 g, 188 mmol, 2 eq) and 1-diazo-1-dimethoxyphosphoryl-propan-2-one (21.7 g, 113 mmol, 1.2 eq) at 0° C. then the mixture stirred at 0° C. for 2 h. The reaction mixture was poured into H₂O (150 mL) and extracted with MTBE (3×150 mL). The combined organic layers were washed with brine (150 mL), dried (Na₂SO₄), filtered and concentrated under reduced pressure. The residue was purified by column chromatography (SiO₂, Petroleum ether:EtOAc) to give compound 16D (14 g, 77% yield) as a white solid; ¹H NMR (400 MHz, DMSO-d₆) δ 3.56 (s, 3H), 2.89 (s, 1H), 1.70 (s, 12H).

### 1.48 Preparation of Compound 16E

Compound 16D (5.0 g, 26.0 mmol, 1 eq) was taken up in THF (500 mL). The suspension was degassed under reduced pressure and purged with nitrogen several times. The solution was cooled to below −65° C. and 2.5 Mn-BuLi (12.5 mL) was added dropwise over 30 min under a nitrogen atmosphere. The mixture was stirred at −65° C. for 30 min then Boc₂O (6.27 mL, 27.3 mmol) was added at −65° C. The mixture was warmed to 20° C. and stirred for 16 h. The mixture was cooled to 0° C. then NH₄Cl (50 mL) was added and stirred at 0° C. for 10 min. The mixture was stirred at 20° C. for 30 min. The aqueous phase extracted with EtOAc (3×200 mL) and the combined organic phase was washed with brine (2×100 mL), dried (Na₂SO₄), filtered, and concentrated under reduced pressure. The residue was purified by column chromatography (SiO₂, Petroleum ether:EtOAc) to give compound 16E (2.4 g, 32% yield) as a white solid; ¹H NMR (400 MHz, DMSO-d₆) δ 3.57 (s, 3H), 1.80-1.66 (m, 12H), 1.41 (s, 9H).

### 1.49 Preparation of Compound 16F

To a mixture of compound 16E (2.20 g, 7.52 mmol, 1 eq) in THF (20 mL) and H₂O (5 mL) was added LiOH·H₂O (316 mg, 7.52 mmol, 1 eq) at 20° C. under a nitrogen atmosphere. The mixture was stirred at 20° C. for 16 h. The reaction mixture was poured into water (50 mL) and the aqueous phase was extracted with EtOAc (150 mL). The aqueous phase adjusted with 1M HCl to pH=4-5 and extracted with EtOAc (2×150 mL). The combined organic phase was washed with brine (2×100 mL), dried (Na₂SO₄), filtered and concentrated under reduced pressure to give compound 16F (1.5 g, 72% yield) as a white solid; LCMS [M−H]: 277.1; ¹H NMR (400 MHz, DMSO-d6) δ 12.14 (br s, 1H), 1.72 (q, J=9.6 Hz, 12H), 1.42 (s, 9H).

### 1.50 Preparation of Compound 16G

To a mixture of compound 16F (650 mg, 2.34 mmol, 1 eq) in DCM (8 mL) was added oxalyl chloride (307 μL, 3.50 mmol, 1.5 eq) and DMF (18 μL, 234 μmol) at 20° C. under a nitrogen atmosphere. The mixture was stirred at 20° C. for 1 h and concentrated under reduced pressure to give crude compound 16G (1.39 g) as a white solid.

### 1.51 Preparation of Intermediate 16

To a mixture of 5-amino-1,3,4-thiadiazole-2-sulfonamide (838 mg, 2.32 mmol, 1 eq) in NMP (8 mL) was added pyridine (375 μL, 4.65 mmol) and compound 16G (690 mg, 2.32 mmol, 1 eq) dropwise over 30 min at 0° C. under a nitrogen atmosphere. The mixture was stirred at 20° C. for 16 h, H₂O (40 mL) added at 20° C., the mixture filtered and the filter cake dried under reduced pressure. The crude product was washed with MTBE (2× 10 mL) to give crude Intermediate 16 (2.3 g) as a light brown solid; LCMS [M+H]: 441.1; ¹H NMR (400 MHz, DMSO-d₆) δ 12.69 (brs, 1H), 8.30 (s, 2H), 1.91-1.74 (m, 12H), 1.49-1.33 (m, 9H).

### A17. Synthesis of Intermediate 17

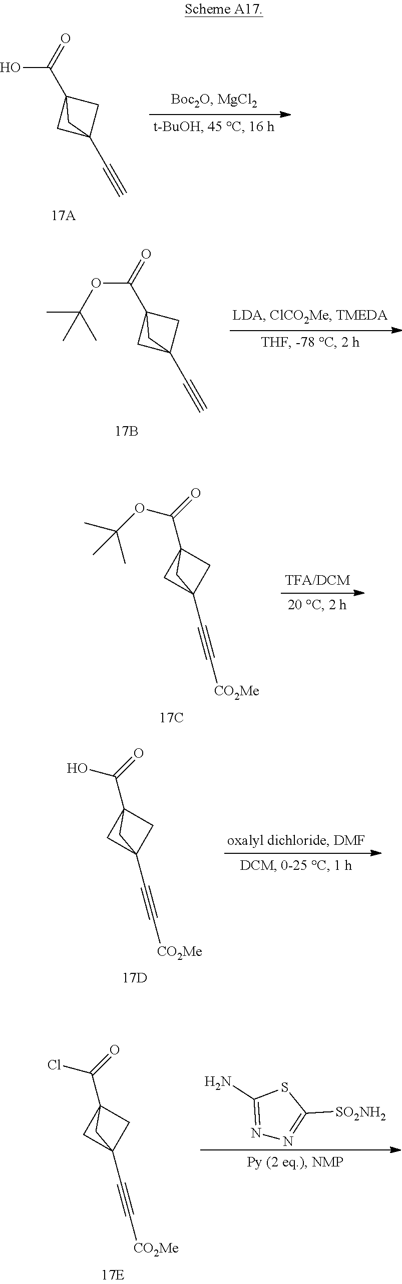

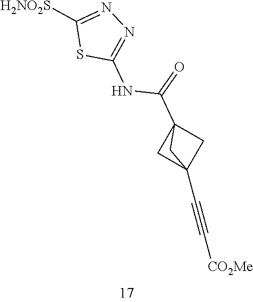 [SMILES: COC(=O)C#CC12CC(C(=O)Nc3nnc(S(N)(=O)=O)s3)(C1)C2]

### 1.52 Preparation of Compound 17B

To a solution of compound 17A (2.95 g, 21.7 mmol) in t-BuOH (50 mL) was added MgCl₂ (89 μL, 2.17 mmol) and Boc₂O (5.97 mL, 26 mmol). The mixture was stirred at 45° C. for 16 h, quenched by addition of NaHCO₃ (20 mL) at 25° C., the combined organic layers washed with DCM (60 mL), dried (Na₂SO₄), filtered and concentrated under reduced pressure. The residue was purified by flash chromatography (SiO₂, Petroleum ether:EtOAc) to give compound 17B (5.0 g, 60% yield) as a white solid; ¹H NMR (400 MHz, CDCl₃) δ=2.28 (s, 6H), 2.11 (s, 1H), 1.44 (s, 9H).

### 1.53 Preparation of Compound 17C

2M LDA (4.94 mL) was added slowly to a solution of compound 17B (1.0 g, 4.94 mmol) in THF (100 mL) and TMEDA (75 μL, 494 μmol) at −78° C. and stirred at −78° C. for 1 h. Methyl carbonochloridate (382 μL, 4.94 mmol) was added at −78° C. and the mixture stirred at −78° C. for 1 h. The reaction mixture was quenched by addition of NH₄Cl (100 mL) at 20° C. for 30 min, and extracted with EtOAc (2×200 mL). The combined organic layers were dried (Na₂SO₄), filtered and concentrated under reduced pressure. The residue was purified by flash chromatography (SiO₂, EtOAc:Petroleum ether) to give compound 17C (5.3 g, 86% yield) as a white solid; ¹H NMR (400 MHz, CDCl₃) δ 3.77 (s, 3H) 2.34 (s, 6H) 1.43 (s, 9H).

### 1.54 Preparation of Compound 17D

A solution of compound 17C (3.0 g, 12 mmol) in DCM (15 mL) and TFA (15 mL) was stirred at 20° C. for 2 h. The reaction mixture was concentrated under reduced pressure to give crude compound 17D (4.4 g, TFA) as a white solid that was used without further purification; ¹H NMR (400 MHz, CDCl₃) δ 3.78 (s, 3H) 2.44 (s, 6H).

### 1.55 Preparation of Compound 17E

To a solution of compound 17D (3.3 g, 17.0 mmol) in DCM (33 mL) was added DMF (131 μL 1.70 mmol). Oxalyl dichloride (1.79 mL, 20.4 mmol) was added at 0° C., the mixture stirred at 25° C. for 1 h and concentrated under reduced pressure to give compound 17E (3.6 g, 99% yield) as a white solid.

### 1.56 Preparation of Intermediate 5

To a solution of intermediate 17E (3.1 g, 16.9 mmol) in NMP (60 mL) was added Py (2.73 mL, 34 mmol). 5-Amino-1,3,4-thiadiazole-2-sulfonamide (3.6 g, 16.9 mmol) in NMP (60 mL) was added to the mixture under N₂ at 0° C. The mixture was stirred at 25° C. for 1 h, the crude product triturated with H₂O at 25° C. for 15 min and filtered. The filter cake was concentrated under reduced pressure to give intermediate 17 (4.0 g, 66% yield) as a white solid; LCMS [M+H]+=357.2; ¹H NMR (400 MHz, DMSO-d₆) δ 13.24 (br s, 1H), 8.34 (s, 2H), 3.71 (s, 3H), 2.52 (s, 6H).

Intermediates in table 6.4 were prepared according to literature procedures.

TABLE 6.4

| Compound | Structure |
|---|---|
| 17.1 | 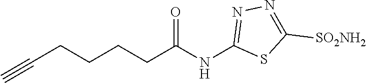 [SMILES: C#CCCCCC(=O)Nc1nnc(S(N)(=O)=O)s1] |
| 17.2 | 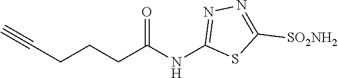 [SMILES: C#CCCCC(=O)Nc1nnc(S(N)(=O)=O)s1] |
| 17.3 | 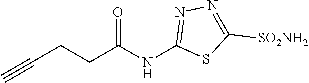 [SMILES: C#CCCC(=O)Nc1nnc(S(N)(=O)=O)s1] |

### B1. Synthesis of Intermediate 30

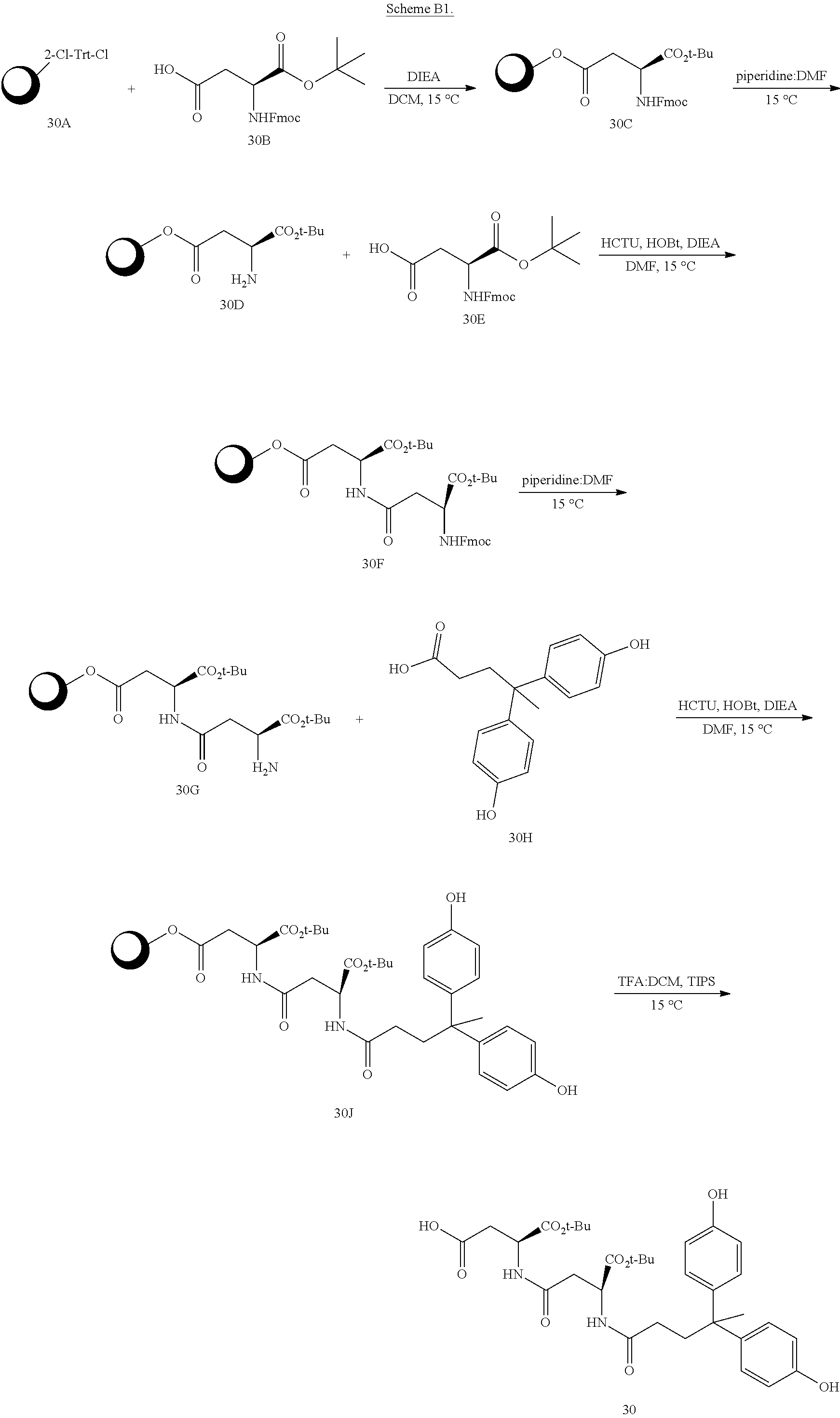

### 2.1 Synthesis of Compound 30C

To a mixture of 2-Cl-Trt-Cl resin (110 g, 110 mmol/g) in DCM (1000 mL) was added a solution of compound 30B (45.3 g, 110 mmol, 1 eq), DIEA (38.3 mL, 220 mmol, 2 eq) in DCM (500 mL) at 15° C. The mixture was shaken at 15° C. for 16 h under a nitrogen atmosphere. The reaction mixture was filtered, the filter cake washed with DCM (3×1000 mL), DMF (2×1000 mL), MeOH (3×1000 mL) and DMF (2×1000 mL), the resin dried with nitrogen to give compound 30C that was used directly in the next reaction.

### 2.2 Synthesis of Compound 30D

A mixture of compound 30C in piperidine (200 mL) and DMF (800 mL) was shaken at 15° C. for 10 min under a nitrogen atmosphere and the mixture filtered. The procedure was repeated four times, the cake washed with DCM (2×1000 mL) and DMF (2×1000 mL), the resin dried with nitrogen to give compound 30D that was used directly in the next reaction.

### 2.3 Synthesis of Compound 30F

A mixture of compound 30E (61.7 g, 150 mmol, 1.5 eq), HCTU (62.1 g, 150 mmol, 1.5 eq), HOBt (20.3 g, 150 mmol, 1.5 eq) and DIEA (29.6 mL, 170 mmol, 1.7 eq) in DMF (1000 mL) was stirred at 15° C. for 30 min. The above mixture was added to a solution of Compound 30D in DMF (1000 mL) and the mixture shaken at 15° C. for 2 h. The mixture was filtered, the cake washed with DMF (1000 mL), DCM (3×1000 mL) and DMF (2×1000 mL), the resin dried with nitrogen to give compound 30F that was used directly in the next reaction.

### 2.4 Synthesis of Compound 30G

A mixture of compound 30F in piperidine (200 mL) and DMF (800 mL) was shaken at 15° C. for 10 min under a nitrogen atmosphere and the mixture filtered. This procedure was repeated four times, the cake washed with DCM (2×1000 mL) and DMF (2×1000 mL), the resin dried with nitrogen to give compound 30G that was used directly in the next reaction.

### 2.5 Synthesis of Compound 30J

A mixture of compound 30H (43.0 g, 150 mmol, 1.5 eq), HCTU (62.1 g, 150 mmol, 1.5 eq), HOBt (20.3 g, 150 mmol, 1.5 eq), DIEA (29.6 mL, 1.7 eq) in DMF (1000 mL) was stirred at 15° C. for 30 min. The mixture was added to a solution of compound 30G in DMF (1000 mL) and the mixture shaken at 15° C. for 2 h. The mixture was filtered, the cake washed with DMF (1000 mL), DCM (3×1000 mL) and DMF (2×1000 mL). The resin was dried with nitrogen to give compound 30J that was used directly in the next reaction.

### 2.6 Synthesis of Compound 30

A solution of compound 30J in DCM (1000 mL), TFA (10 mL) and TIPS (10 mL) was shaken at 15° C. for 10 min. This procedure was repeated three times to ensure completion of the cleavage. The resin was washed with THF (1000 mL), the mixture filtered, the combined organic layer washed with NaHCO₃ (34 g in 500 mL H₂O), the organic layer dried (Na₂SO₄), filtered and concentrated under reduced pressure. The residue was purified by column chromatography (SiO₂, petroleum ether:EtOAc) to give compound 30 (50 g) as a yellow solid; LCMS [M+H]: 629.5.

Compounds in table 7.1 were prepared by procedures depicted in Scheme B¹.

TABLE 7.1

| Intermediate | Structure | m/z |
|---|---|---|
| 30.1 | 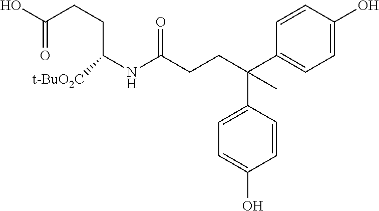 | [M + H]: 472.4 |
| 30.2 | 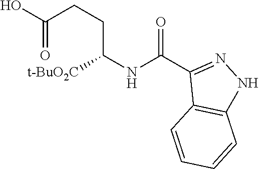 | [M + H]: 348.1 |
| 30.3 | 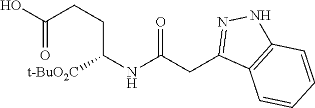 | [M + H]: 362.2 |
| 30.4 | 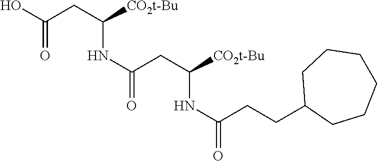 [SMILES: CC(C)(C)OC(=O)[C@H](CC(=O)O)NC(=O)C[C@H](NC(=O)CCC1CCCCCC1)C(=O)OC(C)(C)C] | [M + H]: 513.5 |
| 30.5 | 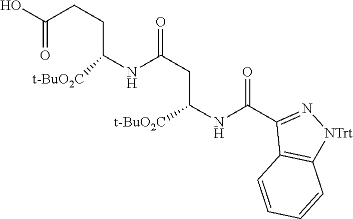 | [M + H]: 716.4 |

### B2. Synthesis of Intermediate 31

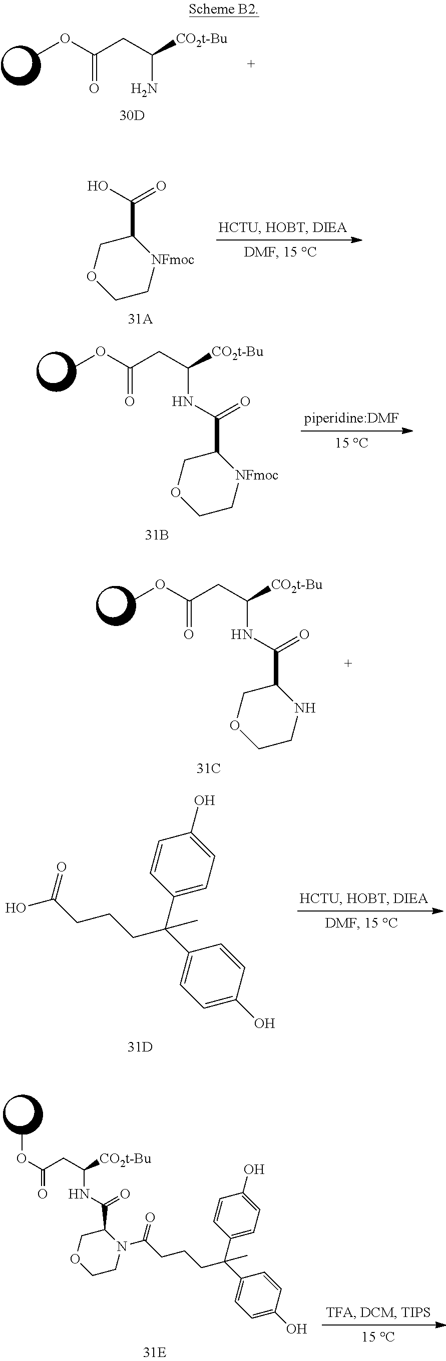

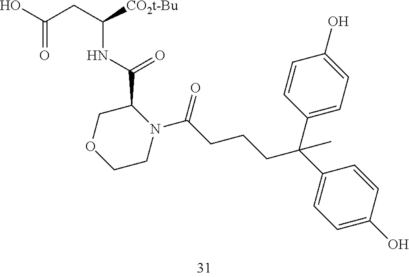 [SMILES: CC(C)(C)OC(=O)[C@H](CC(=O)O)NC(=O)[C@@H]1COCCN1C(=O)CCCC(C)(c1ccc(O)cc1)c1ccc(O)cc1]

### 2.9 Synthesis of Compound 31B

A mixture of compound 31A (8.59 g, 24.3 mmol, 1.5 eq), HOBt (3.28 g, 24.3 mmol, 1.5 eq), HCTU (10.1 g, 24.3 mmol, 1.5 eq) and DIEA (4.80 mL, 27.5 mmol, 1.7 eq) in DMF (50 mL) was stirred at 15° C. for 30 min. The mixture was added to a solution of compound 30D in DMF (50 mL), and the mixture shaken at 15° C. for 2 h. The mixture was filtered and the cake washed with DMF (100 mL), DCM (3× 100 mL) and DMF (2× 100 mL). The resin was dried with nitrogen to give compound 31B which was used directly in the next reaction.

### 2.10 Synthesis of Compound 31C

A mixture of compound 31B in piperidine (20 mL) and DMF (80 mL) was shaken at 15° C. for 10 min under a nitrogen atmosphere and the mixture filtered. The procedure was repeated four times. The cake was washed with DCM (2×100 mL) and DMF (2×100 mL), the resin dried with nitrogen to give compound 31C which was used directly in the next reaction.

### 2.11 Synthesis of Compound 31E

To a mixture of compound 31C (3.1 g, 5.35 mmol, 1 eq) and compound 31D (2.41 g, 8.03 mmol, 1.5 eq) in DMF (50 mL) was added DIEA (1.59 mL, 9.10 mmol, 1.7 eq), HOBt (1.09 g, 8.03 mmol, 1.5 eq) and HCTU (3.32 g, 8.03 mmol, 1.5 eq). The mixture was shaken at 25° C. for 1 h. The mixture was filtered. The cake was washed with DMF (100 mL), DCM (3×100 mL) and DMF (2×100 mL). The resin dried with nitrogen to give compound 31B which was used directly in the next reaction.

### 2.12 Synthesis of Compound 31

To a solution of compound 31E (4.6 g, 5.34 mmol, 1 eq) in DCM (100 mL) was added TFA (1 mL) and TIPS (1 mL). The reaction was shaken for 10 min at 15° C. and the mixture filtered. The procedure was repeated three times. THF (100 mL) was added and shaken for 10 min at 15° C. The mixture was filtered, washed with NaHCO₃ (3.4 g in 50 mL H₂O), the organic layer dried (Na₂SO₄), filtered and concentrated under reduced pressure. The residue was purified by column chromatography (SiO₂, petroleum ether:EtOAc) to give compound 31 (1.4 g, 45% yield) as a yellow solid; LCMS [M+H]: 585.3.

Compounds in table 7.2 were prepared by procedures depicted in scheme B².

TABLE 7.2

| Compound | Structure | m/z |
|---|---|---|
| 31.1 | 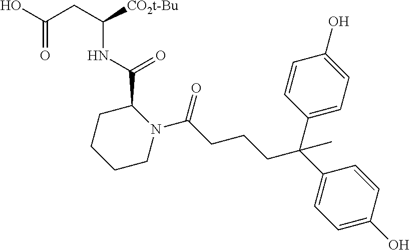 [SMILES: CC(C)(C)OC(=O)[C@H](CC(=O)O)NC(=O)[C@@H]1CCCCN1C(=O)CCCC(C)(c1ccc(O)cc1)c1ccc(O)cc1] | [M + H]: 583.4 |
| 31.2 | 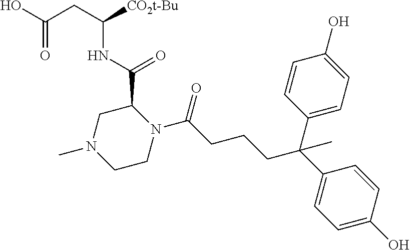 [SMILES: CN1CCN(C(=O)CCCC(C)(c2ccc(O)cc2)c2ccc(O)cc2)[C@H](C(=O)N[C@@H](CC(=O)O)C(=O)OC(C)(C)C)C1] | [M + H]: 598.5 |
| 31.3 | 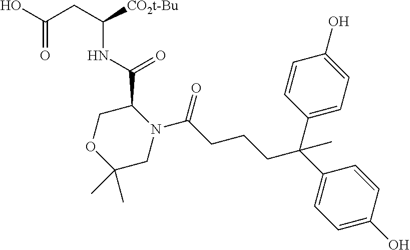 [SMILES: CC(C)(C)OC(=O)[C@H](CC(=O)O)NC(=O)[C@@H]1COC(C)(C)CN1C(=O)CCCC(C)(c1ccc(O)cc1)c1ccc(O)cc1] | [M + H]: 613.4 |
| 31.4 | 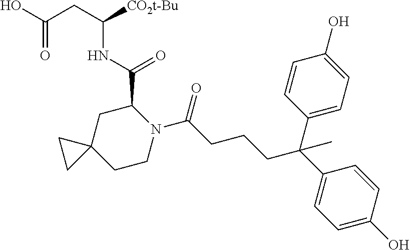 [SMILES: CC(C)(C)OC(=O)[C@H](CC(=O)O)NC(=O)[C@@H]1CC2(CCN1C(=O)CCCC(C)(c1ccc(O)cc1)c1ccc(O)cc1)CC2] | [M + Na]^{:} 631.4 |
| 31.5 | 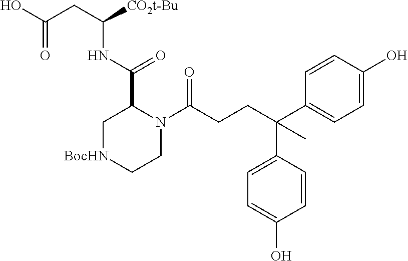 | [M + H]: 670.4 |

### B3. Synthesis of Intermediate 32

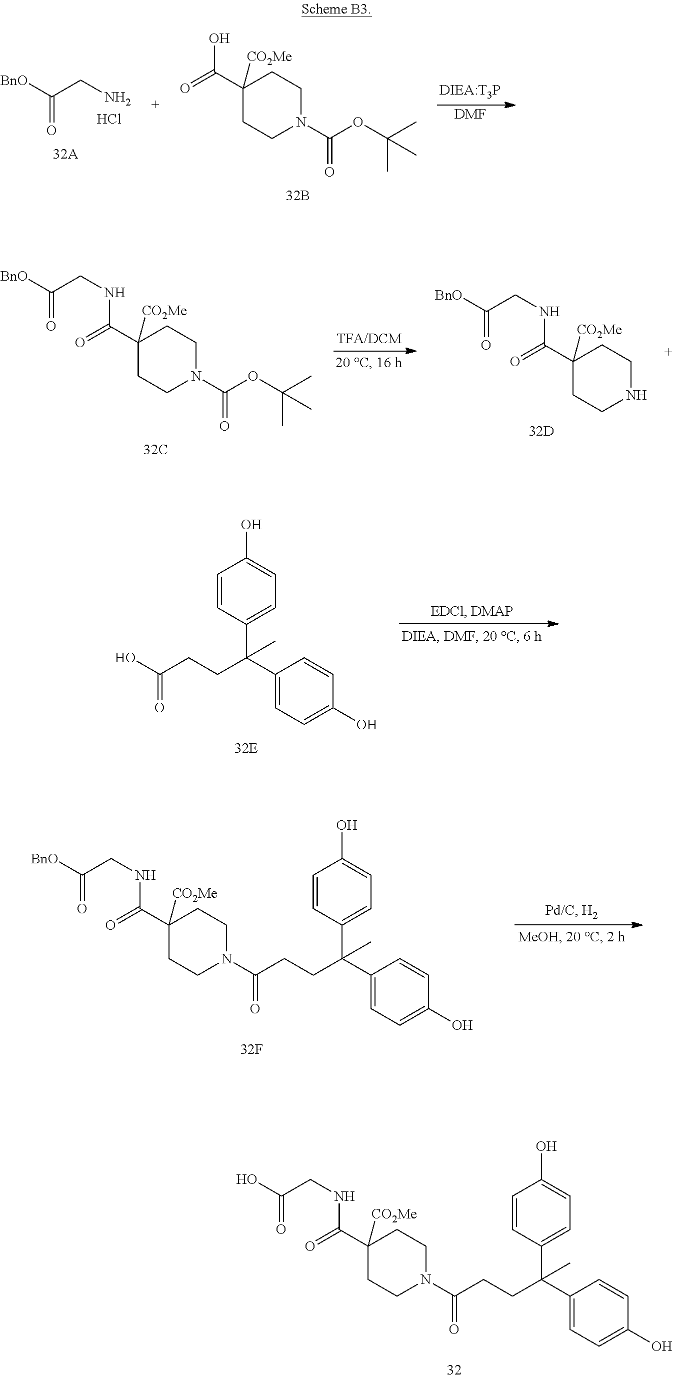

### 2.13 Synthesis of Compound 32C

To a mixture of compound 32B (2.59 g, 9.02 mmol, 1.0 eq) and compound 32A (2.0 g, 9.92 mmol, 1.1 eq) in DMF (30 mL) was added DIPEA (7.85 mL, 45.1 mmol, 5 eq) and T₃P (10.7 mL, 18.0 mmol, 50% purity, 2 eq) at 20° C. under a nitrogen atmosphere. The mixture was stirred at 20° C. for 2 h. The reaction mixture was poured into water (60 mL). The aqueous phase was extracted with EtOAc (3×80 mL). The combined organic phase was washed with brine (2×80 mL), dried (Na₂SO₄), filtered and concentrated under reduced pressure. The crude product was purified by column chromatography (petroleum ether:EtOAc) to give compound 32C (4.0 g, 57% yield, 56% purity) as a colorless oil.

### 2.14 Synthesis of Compound 32D

To a mixture of compound 32C (3.30 g, 7.60 mmol, 1 eq) in DCM (30 mL) was added TFA (10 mL) in one portion at 20° C. under a nitrogen atmosphere. The mixture was stirred at 20° C. for 16 h and concentrated under reduced pressure to give crude compound 32D (2.5 g) as colorless oil; ¹H NMR (400 MHz, METHANOL-d₄) δ 7.47-7.27 (m, 5H), 5.17 (s, 2H), 3.99 (s, 2H), 3.74 (s, 3H), 3.28-3.14 (m, 4H), 2.46-2.24 (m, 4H).

### 2.15 Synthesis of Compound 32F

To a mixture of compound 32D (2.4 g, 7.18 mmol, 1 eq) and compound 32E (2.06 g, 7.18 mmol, 1 eq) in DCM (5 mL) was added DMAP (175 mg, 1.44 mmol, 0.2 eq), DIEA (5.00 mL, 28.7 mmol, 4 eq) and EDCl (2.06 g, 10.8 mmol, 1.5 eq) in one portion at 20° C. under a nitrogen atmosphere. The mixture was stirred at 20° C. for 6 h. The reaction mixture was poured into water (80 mL), the aqueous phase extracted with DCM (3×120 mL), the combined organic phase washed with brine (2×80 mL), dried (Na₂SO₄), filtered and concentrated under reduced pressure. The crude product was purified by column chromatography (SiO₂, Petroleum ether:EtOAc) to give compound 32F (3.6 g, 69% yield, 83% purity) as a white solid; ¹H NMR (400 MHz, METHANOL-d₄) δ 7.40-7.19 (m, 5H), 7.08-6.97 (m, J=8.6 Hz, 4H), 6.70 (d, J=8.8 Hz, 4H), 5.13 (s, 2H), 4.58 (s, 2H), 3.95 (s, 2H), 3.68 (s, 3H), 3.55-3.40 (m, 2H), 3.11 (q, J=4.8 Hz, 2H), 2.33-2.24 (m, 2H), 2.14-2.04 (m, 2H), 1.96 (br d, J=4.4 Hz, 2H), 1.55 (s, 3H).

### 2.16 Synthesis of Compound 32

To a solution of compound 32F (3.2 g, 5.31 mmol, 1 eq) in MeOH (35 mL) was added Pd/C (600 mg, 5.31 mmol, 10% purity, 1 eq) under a nitrogen atmosphere. The suspension was degassed under vacuum and purged with hydrogen 3 times. The mixture was stirred under a hydrogen atmosphere (15 psi) at 20° C. for 2 h. The reaction mixture was filtered and the filtrate concentrated under reduced pressure to give intermediate 32 (2.6 g, 96% yield) as a white solid; ¹H NMR (400 MHz, METHANOL-d₄) δ 7.02 (d, J=8.6 Hz, 4H), 6.69 (d, J=8.4 Hz, 4H), 3.92-3.83 (m, 2H), 3.80-3.67 (m, 4H), 3.40-3.34 (m, 3H), 3.24-3.10 (m, 1H), 2.39-2.18 (m, 2H), 2.18-1.97 (m, 6H).

### B4. Synthesis of Intermediate 33

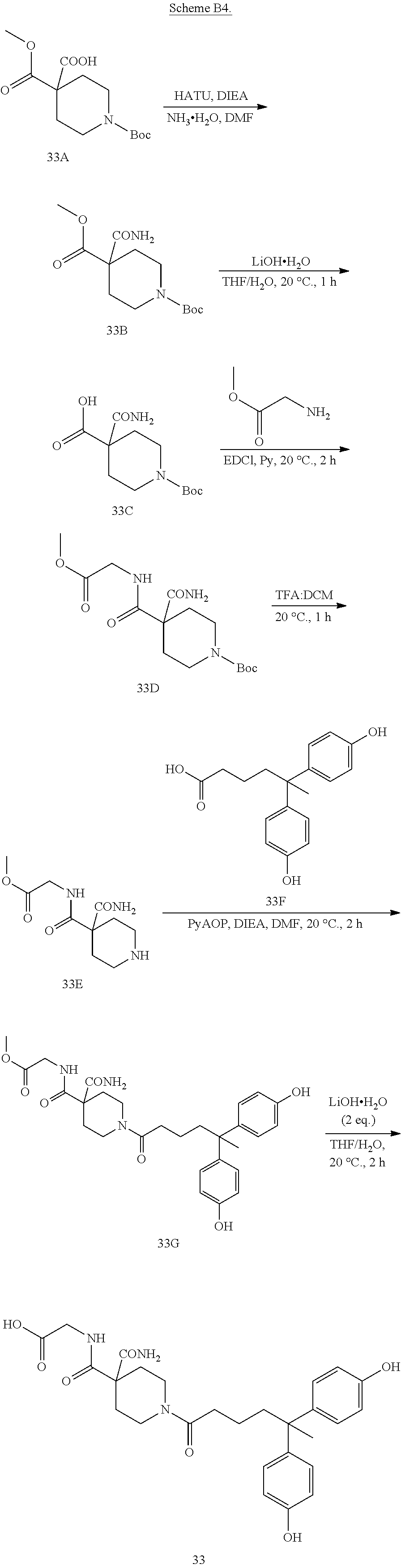

### 2.17 Synthesis of Compound 33B

To a solution of compound 33A (60 g, 209 mmol, 1 eq) in DMF (500 mL) was added HATU (83 g, 219 mmol, 1.1 eq) and DIEA (109 mL, 627 mmol, 3 eq). The mixture was stirred at 20° C. for 0.5 h, NH₃·H₂O (34.5 mL, 251 mmol, 1.2 eq) added at 20° C. and stirred at 20° C. for 1 h. The reaction mixture was extracted with EtOAc (4×500 mL). The combined organic layers were washed with (3×200 mL), dried (Na₂SO₄), filtered and concentrated under reduced pressure. The residue was purified by column chromatography (SiO₂, DCM:MeOH) to give compound 33B (60 g, 92% yield) as a white solid; ¹H NMR (400 MHz, DMSO-d₆) δ 7.45-7.14 (m, 2H), 3.65 (s, 3H), 3.52-3.39 (m, 2H), 3.10 (br s, 2H), 1.98-1.80 (m, 4H), 1.48-1.29 (m, 9H).

### 2.18 Synthesis of Compound 33C

To a solution of compound 33B (50 g, 175 mmol, 1 eq) in THF (300 mL) and H₂O (100 mL) was added LiOH. H₂O (14.7 g, 349 mmol, 2 eq) at 20° C. The mixture was stirred at 20° C. for 1 h and concentrated under reduced pressure to remove organics. IN HCl was added to adjust reaction mixture to pH=2-3, filtered and concentrated under reduced pressure to give crude compound 33C (57 g) as a white solid; ¹H NMR (400 MHz, DMSO-d₆) δ 12.91-12.68 (m, 1H), 7.19 (s, 2H), 3.40 (ddd, J=4.0, 6.2, 13.4 Hz, 2H), 3.15 (br d, J=8.8 Hz, 2H), 1.96-1.78 (m, 4H), 1.38 (s, 9H).

### 2.19 Synthesis of Compound 33D

To a solution of compound 33C (55 g, 101 mmol, 1 eq) and methyl glycinate (27.9 g, 111 mmol, 1.1 eq) in pyridine (400 mL) was added EDCl (46.5 g, 121 mmol, 1.2 eq) at 20° C. The mixture was stirred at 20° C. for 2 h and concentrated under reduced pressure. The residue was purified by column chromatography (SiO₂, DCM:MeOH) to give crude compound 33D (66.5 g) as a white solid; ¹H NMR (400 MHz, DMSO-d₆) δ 8.13 (br t, J=5.6 Hz, 1H), 7.30-6.99 (m, 2H), 3.82 (brd, J=5.5 Hz, 2H), 3.62 (s, 3H), 3.50-3.37 (m, 2H), 3.16 (br s, 2H), 2.08-1.91 (m, 2H), 1.88-1.73 (m, 2H), 1.39 (s, 9H).

### 2.20 Synthesis of Compound 33E

To a solution of compound 33D (60 g, 175 mmol, 1 eq) in DCM (300 mL) was added TFA (100 mL) at 20° C. The mixture was stirred at 20° C. for 1 h and concentrated under reduced pressure. The crude product was triturated with THF at 20° C. for 10 min to give compound 33E (53 g, 82% yield, TFA) as a white solid; ¹H NMR (400 MHz, DMSO-d₆) δ 8.51-8.37 (m, 2H), 7.52-7.11 (m, 2H), 3.85 (br d, J=5.8 Hz, 2H), 3.64 (s, 3H), 3.11 (br s, 2H), 3.00 (brs, 2H), 2.24 (br d, J=13.6 Hz, 2H), 2.11-1.99 (m, 2H).

### 2.21 Synthesis of Compound 33F

To a solution of 5-oxohexanoic acid (92 mL, 768 mmol, 1 eq) in H₂O (24.9 mL, 1.38 mol, 1.8 eq) was added phenol (135 mL, 1.54 mol, 2 eq), the mixture cooled to 0° C., H2SO₄ (73.7 mL, 1.38 mol, 1.8 eq) was added dropwise for 15 min at 0° C., the reaction stirred at 50° C. for 96 h, poured into water (2000 mL) and stirred for 10 minutes. The aqueous phase was extracted with EtOAc (3×2000 mL). The combined organic phase was washed with water (6×1000 mL), combined aqueous phase extracted with ethyl acetate (6×1000 mL), organics dried (Na₂SO₄), filtered and concentrated under reduced pressure. The residue was purified by column chromatography (SiO₂, Petroleum ether/EtOAc) and further purified by prep-HPLC (column: Phenomenex Titank C18 Bulk 250*150 mm 10u; mobile phase: [H₂O (10 mM NH₄HCO₃)-ACN]; gradient: 1%-25% B over 20.0 min) to give compound 33F (128 g, 18% yield) as a white solid; LCMS [M−H]: 299.0; ¹H NMR (400 MHz, DMSO-d₆) δ 11.95 (br s, 1H), 9.13 (br s, 2H), 6.94 (d, J=8.6 Hz, 4H), 6.64 (d, J=8.6 Hz, 4H), 2.16 (br t, J=7.4 Hz, 2H), 2.03-1.85 (m, 2H), 1.48 (s, 3H), 1.35-1.16 (m, 2H).

### 2.22 Synthesis of Compound 33G

To a solution of compound 33E (40 g, 112 mmol, 1 eq, TFA) and compound 33F (33.6 g, 112 mmol, 1 eq) in DMF (300 mL) was added DIEA (58.5 mL, 336 mmol, 3 eq) and PYAOP (61.3 g, 118 mmol, 1.1 eq) at 20° C. The mixture was stirred at 20° C. for 2 h, H₂O (600 mL) added and then the mixture extracted with EtOAc (3×300 mL). The combined organic layers were washed with brine (3×150 mL), dried (Na₂SO₄), filtered and concentrated under reduced pressure. The residue was purified by column chromatography (SiO₂, DCM:MeOH) to give compound 33G (60 g, 89% yield, 87% purity) as a white solid; ¹H NMR (400 MHz, DMSO-d₆) δ 9.15-9.07 (m, 2H), 8.15 (t, J=5.8 Hz, 1H), 7.26-7.04 (m, 2H), 6.97-6.89 (m, 4H), 6.63 (d, J=8.6 Hz, 4H), 3.83 (d, J=5.8 Hz, 2H), 3.62 (s, 3H), 3.47-3.39 (m, 1H), 3.29-3.22 (m, 1H), 3.17 (d, J=5.3 Hz, 2H), 2.29-2.21 (m, 2H), 2.07-1.90 (m, 4H), 1.88-1.72 (m, 2H), 1.48 (s, 3H), 1.26 (d, J=1.1 Hz, 2H).

### 2.23 Synthesis of Intermediate 33

To a solution of compound 33G (40 g, 76.1 mmol, 1 eq) in THF (120 mL) and H₂O (40 mL) was added LiOH·H₂O (6.39 g, 152 mmol, 2 eq) at 20° C. The mixture was stirred at 20° C. for 2 h. LCMS showed Compound 33G was consumed completely and one main peak with desired mass was detected. The reaction mixture was concentrated under reduced pressure to remove THF. The pH was adjusted to 2-3 with 1N HCl and concentrated under reduced pressure to give intermediate 33 (36 g, 92% yield) as a white solid; ¹H NMR (400 MHz, DMSO-d₆) δ 12.07-13.19 (m, 1H) 9.00-9.36 (m, 2H) 8.09 (br t, J=5.6 Hz, 1H) 7.12-7.25 (m, 2H) 6.93 (d, J=8.6 Hz, 4H) 6.63 (d, J=8.6 Hz, 4H) 3.74 (br d, J=5.75 Hz, 2H) 3.57-3.66 (m, 1H) 3.45 (br s, 1H) 3.19-3.29 (m, 2H) 2.24 (br t, J=7.2 Hz, 2H) 1.89-2.06 (m, 4H) 1.74-1.87 (m, 2H) 1.47 (s, 3H) 1.17-1.30 (m, 2H).

Compounds in table 7.3 were prepared by procedures depicted in scheme B4.

TABLE 7.3

| Compound | Structure | m/z |
|---|---|---|
| 33.1 | 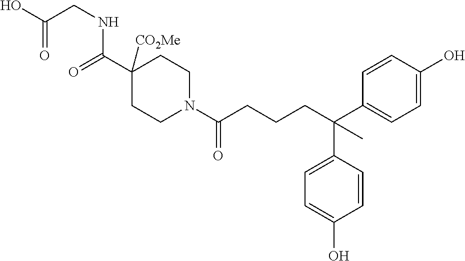 [SMILES: COC(=O)C1(C(=O)NCC(=O)O)CCN(C(=O)CCCC(C)(c2ccc(O)cc2)c2ccc(O)cc2)CC1] | [M + H]: 527.2 |
| 33.2 | 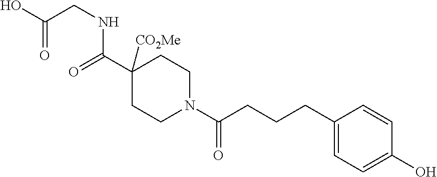 [SMILES: COC(=O)C1(C(=O)NCC(=O)O)CCN(C(=O)CCCc2ccc(O)cc2)CC1] | [M + H]: 407.2 |
| 33.3 | 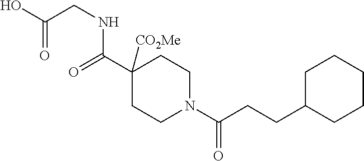 [SMILES: COC(=O)C1(C(=O)NCC(=O)O)CCN(C(=O)CCC2CCCCC2)CC1] | [M + H]: 383.2 |
| 33.4 | 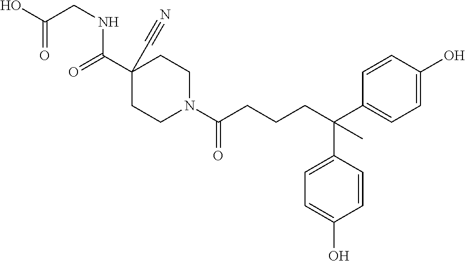 [SMILES: CC(CCCC(=O)N1CCC(C#N)(C(=O)NCC(=O)O)CC1)(c1ccc(O)cc1)c1ccc(O)cc1] | [M + H]: 494.2 |
| 33.5 | 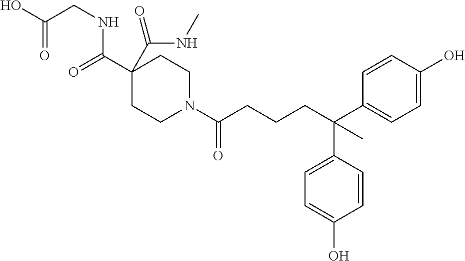 [SMILES: CNC(=O)C1(C(=O)NCC(=O)O)CCN(C(=O)CCCC(C)(c2ccc(O)cc2)c2ccc(O)cc2)CC1] | [M + H]: 526.3 |
| 33.6 | 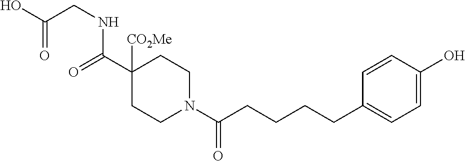 [SMILES: COC(=O)C1(C(=O)NCC(=O)O)CCN(C(=O)CCCCc2ccc(O)cc2)CC1] | [M + H]: 421.3 |

### B5. Synthesis of Intermediate 34

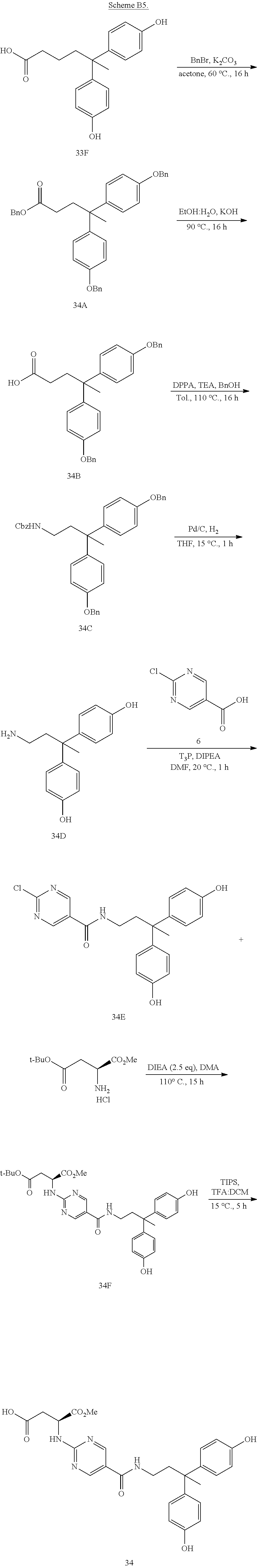

### 2.24 Synthesis of Compound 34A

To a solution of compound 33F (15.0 g, 52.4 mmol, 1 eq) in acetone (150 mL) was added K₂CO₃ (43.4 g, 314 mmol, 6 eq) and bromomethylbenzene (37.3 mL, 314 mmol, 6 eq). The mixture was stirred at 60° C. for 16 h. Additional bromomethylbenzene (37.3 mL, 314 mmol, 6 eq) was added and the mixture stirred at 60° C. for 14 h. The reaction mixture was quenched by addition of H₂O (40 mL) at 15° C., diluted with H₂O (50 mL) and extracted with EtOAc (2×120 mL). The combined organic layers were washed with brine (100 mL), dried (Na₂SO₄), filtered and concentrated under reduced pressure. The residue was purified by column chromatography (SiO₂, petroleum ether:EtOAc) to give compound 34A (18.1 g, 62%) as a white solid; ¹H NMR (400 MHz, DMSO-d₆) δ 7.48-7.27 (m, 15H), 7.07 (d, J=8.9 Hz, 4H), 6.92 (d, J=8.8 Hz, 4H), 5.08-5.00 (m, 6H), 2.37-2.29 (m, 2H), 2.15-2.05 (m, 2H), 1.51 (s, 3H).

### 2.25 Synthesis of Compound 34B

To a solution of compound 34A (17 g, 31 mmol, 1 eq) in H₂O (40 mL) was added KOH (9.42 g, 168 mmol, 5.5 eq) and EtOH (120 mL). The mixture was stirred at 90° C. for 16 h. The reaction mixture was diluted with MTBE (150 mL) and extracted with MTBE (2× 40 mL). To the mixture was added aqueous citric acid solution until the reaction mixture pH=4. The combined organic layers were washed with brine (150 mL), dried (Na₂SO₄), filtered and concentrated under reduced pressure to give compound 34B (14.3 g, 100% yield) as a colorless oil; ¹H NMR (400 MHz, DMSO-d₆) δ 7.49-7.30 (m, 9H), 7.14-7.04 (m, 4H), 6.91 (d, J=8.8 Hz, 4H), 5.09-5.02 (m, 4H), 3.10-3.05 (m, 1H), 2.29-2.21 (m, 2H), 2.02 (br s, 3H), 1.56-1.44 (m, 3H).

### 2.26 Synthesis of Compound 34C

To a solution of compound 34B (13.3 g, 28.5 mmol, 1 eq) in toluene (10 mL) was added TEA (11.9 mL, 85.4 mmol, 3 eq), BnOH (8.9 mL, 85.4 mmol, 3 eq) and DPPA (6.17 mL, 28.5 mmol, 1 eq). The mixture was stirred at 110° C. for 15 h. The reaction mixture was extracted with MTBE (2×100 mL), washed with 1M NaOH (2×150 mL), 0.5 M HCl (200 mL), brine (150 mL), dried (Na₂SO₄), filtered and concentrated under reduced pressure. The residue was purified by column chromatography (SiO₂, petroleum ether:EtOAc) to give compound 34C (10.5 g, 65%) as a colorless oil.

2.27 Synthesis of compound 34D

A suspension of compound 34C (4.7 g, 8.22 mmol, 1 eq) in THF (100 mL) was degassed under vacuum and purged with nitrogen three times. Then Pd/C (2.5 g, 8.22 mmol, 10% purity, 1 eq) was added slowly. The suspension was degassed and purged with hydrogen 3 times and the mixture was stirred at 15° C. for 4 h. The reaction mixture was filtered and the filtrate concentrated under reduced pressure to give crude compound 34D (4.8 g) as a pink solid; ¹H NMR (400 MHz, DMSO-d₆) δ 6.95-6.80 (m, 4H), 6.63 (br d, J=8.5 Hz, 4H), 3.63-3.56 (m, 1H), 2.34-2.26 (m, 2H), 2.08-2.00 (m, 2H), 1.80-1.71 (m, 1H), 1.47 (s, 3H).

### 2.28 Synthesis of Compound 34E

A suspension of compound 34D (4.3 g, 16.7 mmol, 1 eq), 2-chloropyrimidine-5-carboxylic acid (2.65 g, 16.7 mmol, 1 eq), DIEA (14.6 mL, 83.6 mmol, 5 eq) and DMF (40 mL) was degassed under reduced pressure and purged with nitrogen three times. Then T₃P (14.9 mL, 25.1 mmol, 50% purity, 1.5 eq) was added slowly and the mixture stirred at 15° C. for 2 h. The reaction mixture was quenched by addition of H₂O (15 mL) at 20° C., diluted with H₂O (30 mL) and extracted with EtOAc (100 mL). The combined organic layers were washed with brine (50 mL), dried (Na₂SO₄), filtered and concentrated under reduced pressure. The residue was purified by column chromatography (SiO₂, Petroleum ether:EtOAc) to give compound 34E (3.33 g, 50% yield) as a colorless oil; ¹H NMR (400 MHz, DMSO-d₆) δ 9.20-9.15 (m, 2H), 9.05 (s, 2H), 8.75 (t, J=5.3 Hz, 1H), 6.99 (d, J=8.7 Hz, 4H), 6.66 (d, J=8.7 Hz, 4H), 3.12-2.98 (m, 2H), 2.30-2.18 (m, 2H), 1.56 (s, 3H).

### 2.29 Synthesis of Compound 34F

To a solution of compound 34E (2.93 g, 7.36 mmol, 1 eq) in DMA (30 mL) was added DIEA (3.21 mL, 18.4 mmol, 2.5 eq) and 4-(tert-butyl) 1-methyl L-aspartate HCl (1.94 g, 8.10 mmol, 1.1 eq). The mixture was stirred at 110° C. for 16 h. The reaction mixture was quenched by addition of H₂O (5 mL) at 15° C. The residue was purified by column chromatography (SiO₂, Petroleum ether:EtOAc) to give compound 34F (2.88 g, 69%) as a yellow oil; ¹H NMR (400 MHz, DMSO-d₆) δ 9.15 (s, 2H), 8.68 (s, 2H), 8.25 (t, J=5.3 Hz, 1H), 8.04-7.99 (m, 1H), 6.99 (d, J=8.6 Hz, 4H), 6.66 (d, J=8.6 Hz, 4H), 4.91 (dt, J=6.1, 8.0 Hz, 1H), 3.61 (s, 3H), 3.01 (td, J=5.4, 10.4 Hz, 2H), 2.85-2.77 (m, 1H), 2.75-2.68 (m, 1H), 2.24-2.15 (m, 2H), 1.55 (s, 3H), 1.36 (s, 9H).

### 2.30 Synthesis of Compound 34

To a solution of compound 34F (2.5 g, 4.43 mmol, 1 eq) in DCM (18 mL) was added TIPS (18.2 mL, 88.6 mmol, 20 eq) and TFA (6.0 mL, 81.0 mmol). The mixture was stirred at 15° C. for 5 h. The reaction mixture was concentrated under reduced pressure, washed with H₂O (2×25 mL), extracted with EtOAc (2×75 mL), dried (Na₂SO₄), filtered and concentrated under reduced pressure to give compound 34 (2.4 g, crude) as a yellow solid; ¹H NMR (400 MHz, DMSO-d₆) δ 12.54-12.34 (m, 1H), 9.21-9.11 (m, 2H), 8.68 (br s, 2H), 8.25 (brs, 1H), 8.03 (br d, J=8.0 Hz, 1H), 6.99 (br d, J=8.4 Hz, 4H), 6.66 (br d, J=8.3 Hz, 4H), 4.93-4.82 (m, 1H), 3.61 (s, 3H), 3.00 (br s, 2H), 2.89-2.69 (m, 2H), 2.25-2.15 (m, 2H), 1.55 (br s, 3H), 1.06-0.93 (m, 1H).

### B6. Synthesis of Intermediate 35

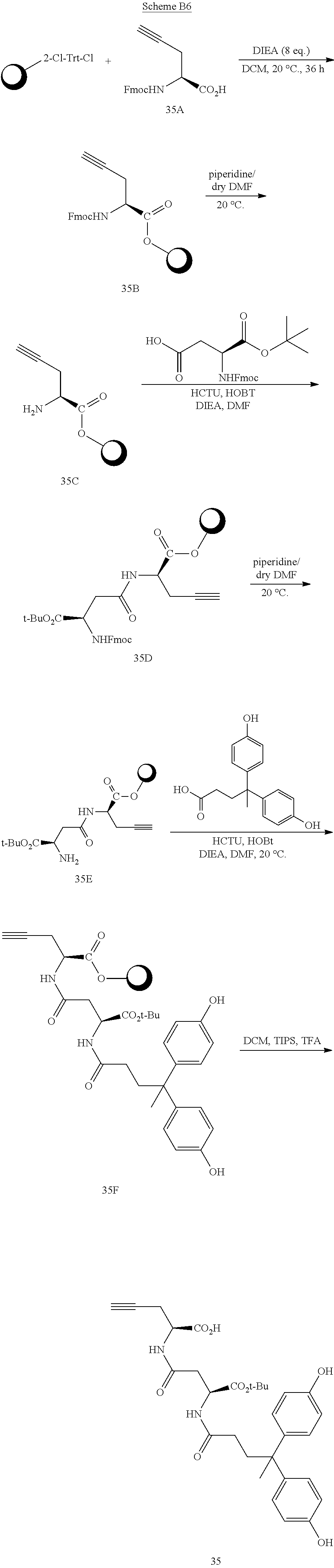

### 2.31 Synthesis of Compound 35B

To a solution of compound 35A (10 g, 29.8 mmol, 1.0 eq) in DCM (100 mL) was added 2-Cl-Trt-Cl resin (9.34 g, 29.8 mmol, 1.0 eq), and DIEA (41.6 mL, 239 mmol, 8 eq). The mixture was stirred at 20° C. for 36 hours under a nitrogen atmosphere. The reaction mixture was filtered, the cake washed with DCM (3×200 mL), DMF (2×200 mL), MeOH (3×200 mL) and DMF (200 mL), the resin dried with nitrogen to give compound 35B which was used directly in the next reaction.

### 2.32 Synthesis of Compound 35C

A mixture of compound 35B (5.0 g, 14.9 mmol, 1.0 eq) in piperidine (20 mL) and DMF (100 mL) was shaken at 20° C. for 10 minutes under a nitrogen atmosphere, and the mixture filtered. The procedure was repeated four times. The cake was washed with DCM (2×100 mL), DMF (2×100 mL), the resin dried with nitrogen to give compound 35C which was used directly in the next reaction.

### 2.33 Synthesis of Compound 35D

To a solution of(S)-3-((((9H-fluoren-9-yl)methoxy) carbonyl)amino)-4-(tert-butoxy)-4-oxobutanoic acid (9.22 g, 22.4 mmol, 1.5 eq) in DMF (50 mL) was added HCTU (9.27 g, 22.4 mmol, 1.5 eq), HOBt (3.03 g, 22.4 mmol, 1.5 eq) and DIEA (7.81 mL, 44.8 mmol, 3 eq). The mixture stirred at 20° C. for 0.5 h. Compound 35C (1.69 g, 14.9 mmol, 1 eq) was added and the mixture stirred at 20° C. for 1 h under a nitrogen atmosphere. The mixture was filtered, the cake washed with DMF (3×100 mL), DCM (5× 100 mL), and DMF (100 mL). The resin was dried with nitrogen to give compound 35D which was used directly in the next reaction.

### 2.34 Synthesis of Compound 35E

A mixture of compound 35D (7.57 g, 14.9 mmol, 1.0 eq) in piperidine (20 mL) and DMF (100 mL) was shaken at 20° C. for 10 minutes under a nitrogen atmosphere, and the mixture filtered. The procedure was repeated four times. The cake was washed with DCM (2×100 mL) and DMF (2×100 mL), the resin was dried with nitrogen to give compound 35E which was used directly in the next reaction.

### 2.35 Synthesis of Compound 35F

To a solution of 4,4-bis(4-hydroxyphenyl)pentanoic acid (4.08 g, 15.0 mmol, 1 eq) in DMF (50 mL) was added HCTU (5.89 g, 15 mmol, 1 eq), HOBt (1.92 mg, 15 mmol, 1 eq) and DIEA (2.98 mL, 17.09 mmol, 1.2 eq). To the mixture was added compound 35F (4.25 g, 14.95 mmol, 1 eq) and the mixture stirred at 20° C. for 1 h under a nitrogen atmosphere. The mixture was filtered, the cake washed with DMF (3×100 mL) and DCM (5×100 mL), the resin dried with nitrogen to give compound 35F which was used directly in the next reaction.

### 2.36 Synthesis of Intermediate 35

A solution of compound 35F (13.2 g, 23.8 mmol, 1 eq) in DCM (100 mL), TFA (1 mL) and TIPS (2.5 mL) was stirred at 20° C. for 10 minutes. The procedure was repeated three times. THF (100 mL) was added and stirred at 20° C. for 10 min. The mixture was filtered, washed with NaHCO₃until the reaction mixture pH=7, the organic layer dried (Na₂SO₄), filtered and concentrated under reduced pressure to give crude Intermediate 35 (3.24 g); LCMS [M+H]: 553.4; ¹H NMR (400 MHz, DMSO-d₆) δ 12.89 (br s, 1H), 9.15 (s, 2H), 8.28 (d, J=8.0 Hz, 1H), 7.92 (d, J=8.0 Hz, 1H), 6.93 (d, J=8.6 Hz, 4H), 6.64 (d, J=8.6 Hz, 4H), 4.44-4.30 (m, 2H), 2.80 (t, J=2.4 Hz, 1H), 2.54 (br t, J=4.8 Hz, 4H), 2.21-2.12 (m, 2H), 1.91-1.80 (m, 2H), 1.46 (s, 3H), 1.36 (s, 9H).

### B7. Synthesis of Intermediate 36

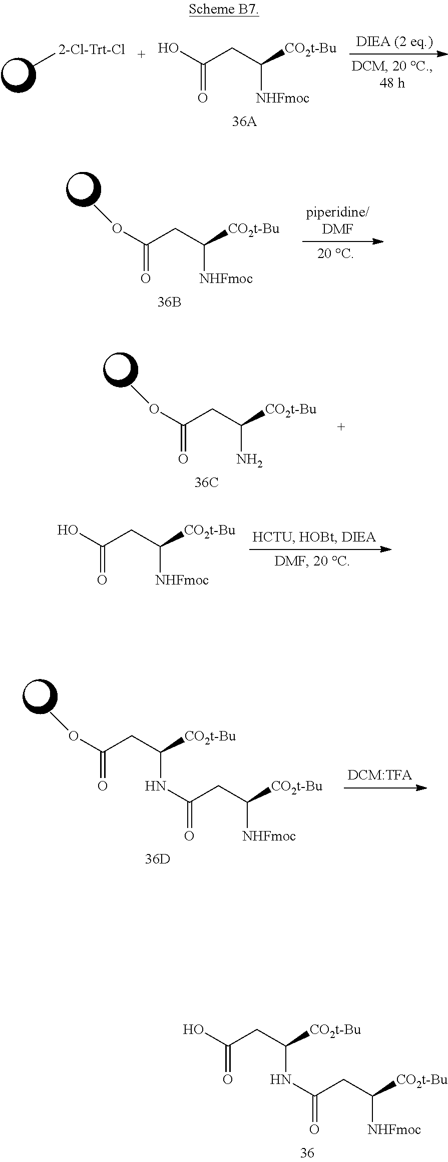

### 2.37 Preparation of Compound 36B

To a solution of compound 36A (30 g, 72.9 mmol, 1.0 eq) in DCM (300 mL) was added 2-Cl-Trt-Cl resin (22.8 g, 72.9 mmol, 1 eq), and DIEA (25.4 mL, 146 mmol, 2 eq). The mixture was stirred at 20° C. for 48 h under a nitrogen atmosphere. The reaction mixture was filtered and the cake washed with DCM (3×300 mL), DMF (2×300 mL), MeOH (3×300 mL) and DMF (300 mL). The resin was dried with nitrogen to give compound 36B which was used directly in the next reaction.

### 2.38 Preparation of Compound 36C

A mixture of compound 36B (30 g, 72.9 mmol, 1.0 eq) in piperidine (20 mL) and DMF (100 mL) was shaken at 20° C. for 10 min under a nitrogen atmosphere and the mixture filtered. A sample of the filtrate (0.1 mL) was added to water (0.5 mL) and the mixture turned to a white suspension. This procedure was repeated four times until the mixture of the filtrate and water was a transparent liquid. The cake was then washed with DCM (2× 300 mL), DMF (2× 300 mL), the resin dried with nitrogen to give compound 36C which was used directly in the next reaction.

### 2.39 Preparation of Compound 36D

To a solution of(S)-3-((((9H-fluoren-9-yl)methoxy) carbonyl)amino)-4-(tert-butoxy)-4-oxobutanoic acid (45.0 g, 109 mmol, 1.5 eq) in DMF (240 mL) was added HCTU (45 g, 109 mmol, 1.5 eq), HOBt (14.8 g, 109 mmol, 1.5 eq) and DIEA (25.4 mL, 146 mmol, 2 eq). The mixture was stirred at 20° C. for 0.5 h, compound 36C (13.8 g, 72.9 mmol, 1 eq) added and the mixture stirred at 20° C. for 1 h under a nitrogen atmosphere. The mixture was filtered, the cake washed with DMF (3×300 mL), DCM (5×300 mL), and DMF (300 mL), the resin dried with nitrogen to give compound 36D which was used directly in the next reaction.

### 2.40 Preparation of Intermediate 36

To a solution of compound 36D (42.5 g, 72.9 mmol, 1.0 eq) in DCM (100 mL) and TFA (1 mL) was stirred at 20° C. for 10 min. This procedure was repeated three times to ensure completion of the cleavage. DCM (300 mL) was added to the reaction mixture and the reaction stirred at 20° C. for 10 min. The mixture was filtered, the filtrate diluted with MeOH and washed with saturated NaHCO₃ solution (pH=7), the organic layer dried (Na₂SO₄), filtered and concentrated to give Intermediate 36 (40 g) as a white solid; LCMS [M+H]: 583.4; ¹H NMR (400 MHz, DMSO-d₆) δ 8.06 (br d, J=7.8 Hz, 1H), 7.66 (d, J=7.6 Hz, 2H), 7.47 (br d, J=7.2 Hz, 2H), 7.27 (br d, J=8.4 Hz, 1H), 7.22-7.15 (m, 2H), 7.13-7.06 (m, 2H), 4.29-4.20 (m, 1H), 4.12-3.93 (m, 4H), 2.40-2.30 (m, 4H), 1.14 (br d, J=2.8 Hz, 18H).

### B8. Synthesis of Compound 37

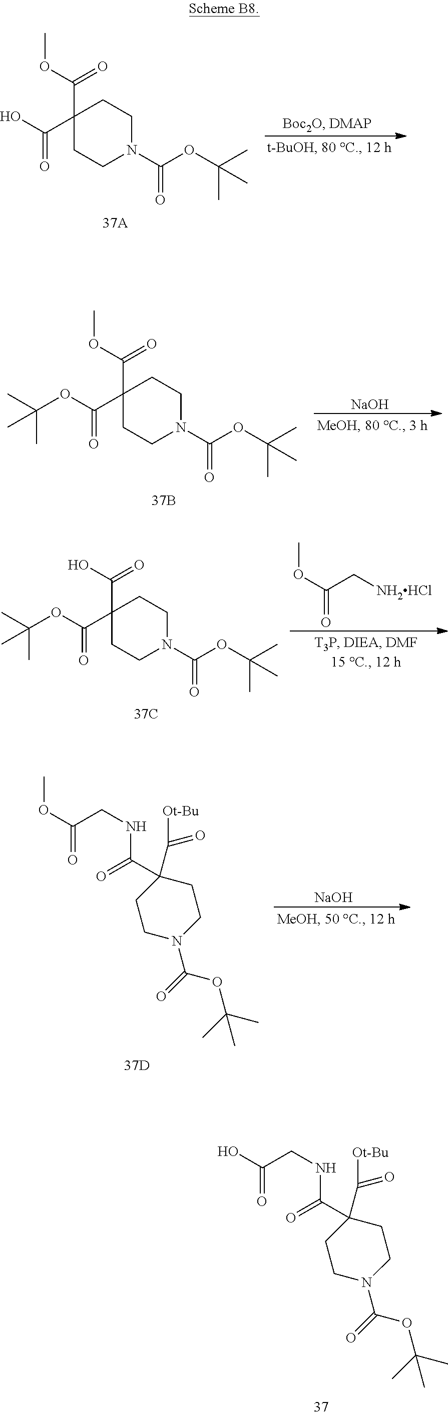

### 2.41 Synthesis of Compound 37B

To a solution of compound 37A (10.0 g, 34.8 mmol, 1 eq) and Boc₂O (20 mL, 87.1 mmol, 2.50 eq) in t-BuOH (20 mL) was added DMAP (1.28 g, 10.4 mmol, 0.3 eq) at 15° C. under a nitrogen atmosphere. The mixture was stirred at 80° C. for 12 h then concentrated under reduced pressure and purified by chromatography column (SiO₂, petroleum ether:EtOAc) to give compound 37B (11 g, 92% yield) as a white solid.

### 2.42 Synthesis of Compound 37C

To a solution of compound 37B (11 g, 32.0 mmol, 1 eq) in MeOH (12 mL) was added 3M NaOH (10.7 mL, 1 eq) at 15° C. under a nitrogen atmosphere. The mixture was stirred at 80° C. for 3 h and concentrated under reduced pressure. The aqueous solution was adjusted to pH=2 with 1M HCl (30 mL), filtered and the filter cake concentrated under reduced pressure to give compound 37C (10 g, 95% yield) as a white solid that was used directly in the next reaction; ¹H NMR (400 MHz, CHLOROFORM-d) 8 3.56-3.36 (m, 4H), 2.13-1.97 (m, 4H), 1.47 (d, J=3.9 Hz, 18H).

### 2.43 Synthesis of Compound 37D

To a solution of compound 37C (8.8 g, 26.7 mmol, 1 eq) and methyl glycinate hydrochloride (8.39 g, 67 mmol, 2.5 eq, HCl) in DMF (9 mL) were added T₃P (31.8 mL, 53.4 mmol, 50% purity, 2 eq) and DIEA (27.9 mL, 160 mmol, 6 eq) at 15° C. under a nitrogen atmosphere. The mixture was stirred at 15° C. for 12 h and concentrated under reduced pressure. Brine (60 mL) was added. The reaction was extracted with DCM (3×60 mL), the organics dried (Na₂SO₄) and concentrated under reduced pressure. The residue was purified by chromatography column (SiO₂, petroleum ether:EtOAc) to give compound 37D (10 g, 93% yield) as a yellow oil.

### 2.44 Synthesis of Compound 37

To a solution of compound 37D (9.0 g, 22.5 mmol, 1 eq) in MeOH (10 mL) was added 2M NaOH (16.9 mL, 1.5 eq) at 15° C. under a nitrogen atmosphere. The mixture was stirred at 50° C. for 12 h and concentrated under reduced pressure to remove organics. The remaining aqueous solution was adjusted to pH=2 with 1M HCl (15 mL) and extracted with DCM (3×30 mL). The organic phase was dried (Na₂SO₄) and concentrated under reduced pressure to give Intermediate 37 (8.5 g, 98% yield) as a white solid that was used directly in the next reaction.

### B9. Synthesis of Intermediate 38

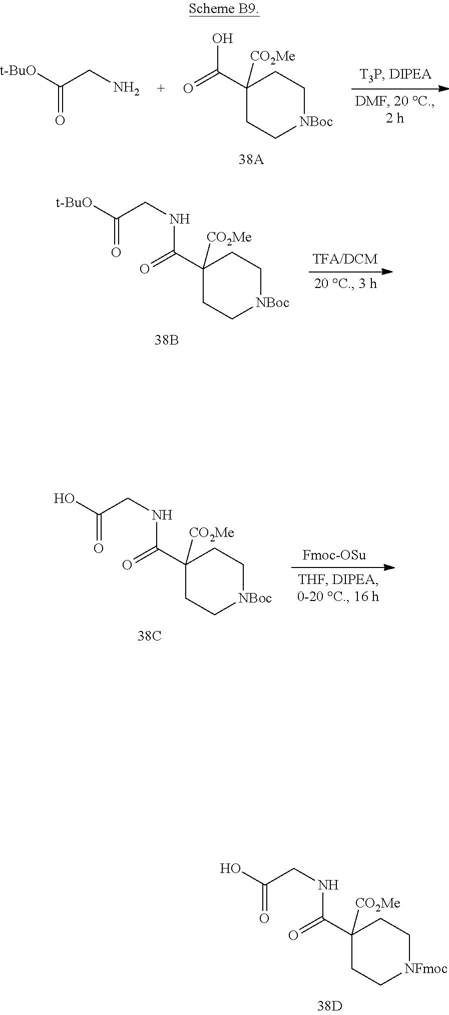

### 2.45 Preparation of Compound 38B

To a solution of tert-butyl glycinate (2.74 g, 21 mmol, 1 eq) in DMF (60 mL) was added compound 38A (6.0 g, 21 mmol, 1 eq) and DIEA (18 mL, 104 mmol, 5 eq). T₃P (18.6 mL, 31.3 mmol, 50% purity, 1.5 eq) was added to the reaction mixture slowly and the reaction mixture stirred at 20° C. for 2 h. The reaction mixture was quenched by the addition of H₂O (20 mL) at 25° C., concentrated under reduced pressure, the residue diluted with H₂O (150 mL) and extracted with EtOAc (300 mL). The combined organic layers were washed with H₂O (100 mL), brine (100 mL), dried (Na₂SO₄), filtered and concentrated under reduced pressure. The residue was purified by column chromatography (SiO₂, petroleum ether:EtOAc) to give compound 38B (14 g, 84% yield) as an off-white solid; ¹H NMR (400 MHz, CHLOROFORM-d) δ 6.48 (br s, 1H), 3.91 (d, J=4.8 Hz, 2H), 3.84-3.68 (m, 5H), 3.11 (br s, 2H), 2.25-2.10 (m, 2H), 2.10-1.94 (m, 2H), 1.49-1.42 (m, 18H).

### 2.46 Preparation of Compound 38C

To a mixture of compound 38B (12 g, 30 mmol, 1 eq) in DCM (90 mL) was added TFA (30 mL) at 20° C. under a nitrogen atmosphere. The mixture was stirred at 20° C. for 3 h and concentrated under reduced pressure to give compound 38C (8.5 g, crude) as a colorless oil; LCMS [M+H]: 245.0; ¹H NMR (400 MHz, METHANOL-d₄) δ 3.91 (s, 3H), 3.30-3.16 (m, 5H), 2.42-2.19 (m, 6H).

### 2.47 Preparation of Intermediate 38D

To a mixture of compound 38C (8.5 g, 34.8 mmol, 1 eq) in THF (80 mL) was added DIEA (30 mL, 174 mmol, 5 eq) at 0° C. under a nitrogen atmosphere. The mixture was stirred at 0° C. for 15 min, then Fmoc-OSu (36.4 g, 108 mmol, 3.1 eq) added at 0° C. The mixture was stirred at 20° C. for 16 h, poured into NaHCO₃ (20 mL) and the aqueous phase extracted with EtOAc (3×100 mL). The aqueous phase was adjust to pH=3-4 with 1M HCl, the mixture extracted with EtOAc (3×200 mL), the combined organic phase washed with brine (2×100 mL), dried (Na₂SO₄), filtered and concentrated under reduced pressure to give intermediate 38 (12 g, 74% yield) as a white solid; LCMS [M+H]: 467.3; ¹H NMR (400 MHz, DMSO-d₆) δ 8.15 (t, J=5.8 Hz, 1H), 7.89 (d, J=7.6 Hz, 2H), 7.62 (d, J=7.6 Hz, 2H), 7.45-7.31 (m, 4H), 4.40 (br d, J=6.0 Hz, 2H), 4.34-4.20 (m, 1H), 3.72 (d, J=5.8 Hz, 2H), 3.66 (s, 3H), 3.35 (m, 2H), 3.23 (m, 2H), 1.95-1.72 (m, 4H).

### B10. Synthesis of Intermediate 39

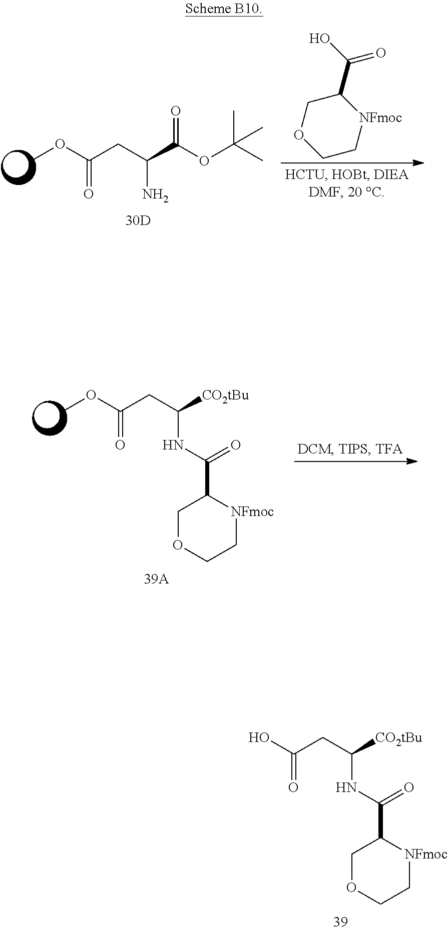

### 2.50 Preparation of Compound 39A

To a solution of(S)-4-(((9H-fluoren-9-yl)methoxy)carbonyl)morpholine-3-carboxylic acid (11.2 g, 31.6 mmol, 1.3 eq) in DMF (100 mL) was added HCTU (10.1 g, 24.3 mmol, 1 eq), HOBt (3.29 g, 24.3 mmol, 1 eq) and DIEA (6.35 mL, 36.5 mmol, 1.5 eq), and the mixture stirred at 20° C. for 0.5 h. Compound 30D (4.6 g, 24.3 mmol, 1 eq) was added and the mixture stirred at 20° C. for 1 h under a nitrogen atmosphere. The mixture was filtered, and the cake washed with DMF (3×100 mL), DCM (5×100 mL) and DMF (100 mL), the resin dried with nitrogen to give compound 39A which was used directly in the next reaction.

### 2.51 Preparation of Intermediate 39

A solution of compound 39A in DCM (100 mL), TFA (1 mL) and TIPS (2.5 mL) was stirred at 15° C. for 10 minutes. This procedure was repeated three times, THF (100 mL) added and stirred at 15° C. for 10 min. The mixture was filtered, the filtrate diluted in MeOH, washed with NaHCO₃ (pH=7), the organic layer dried (Na₂SO₄), filtered and concentrated to give crude Intermediate 39 (8.7 g); LCMS [M+H]: 525.3.

### B11. Synthesis of Intermediate 40

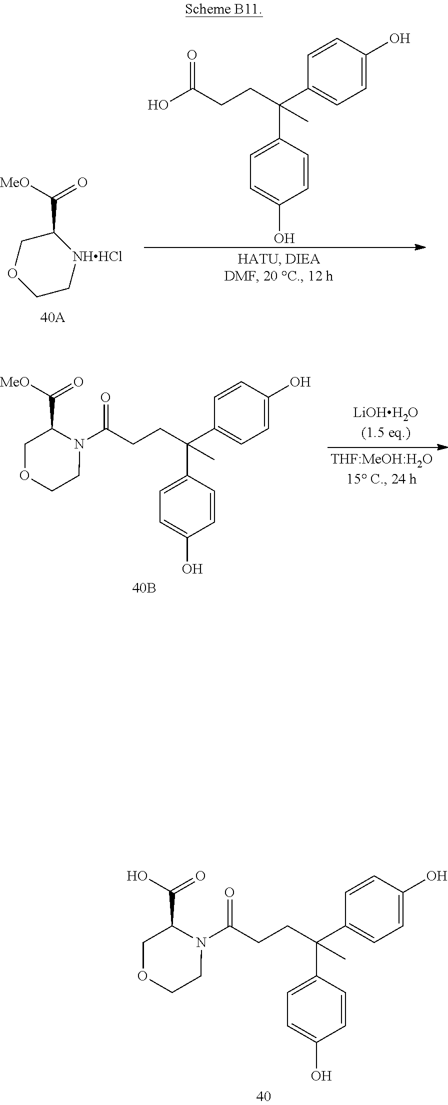

### 2.54 Synthesis of Compound 40B

To a solution of compound 40A (2.0 g, 11.0 mmol, 1 eq) and 4,4-bis(4-hydroxyphenyl)pentanoic acid (3.2 g, 11.0 mmol, 1 eq) in DMF (30 mL) was added HATU (4.19 g, 11.0 mmol, 1 eq) and DIEA (9.59 mL, 55.1 mmol, 5 eq). The mixture was stirred at 20° C. for 12 h. The reaction mixture was diluted with EtOAc (45 mL), extracted with a saturated NH₄Cl solution (12 mL) and a saturated Na₂CO₃ solution (12 mL). The combined organic layers were washed with brine (9 mL), dried (Na₂SO₄), filtered and concentrated under reduced pressure. The residue was purified by column chromatography (SiO₂, petroleum ether:EtOAc) to give compound 40B (4.5 g, 99% yield) as a white solid; LCMS [M+H]: 414.4.

### 2.55 Synthesis of Intermediate 40

A solution of compound 40B (4.5 g, 10.9 mmol, 1 eq) and LiOH. H₂O (457 mg, 10.9 mmol, 1 eq) in THF (15 mL), MeOH (15 mL) and H₂O (15 mL) was stirred at 15° C. for 24 h. The reaction mixture was concentrated under reduced pressure to remove organics. The aqueous phase was adjusted to pH=5 with 1 M HCl. Precipitated white solids were filtered and the cake dried under reduced pressure to give Intermediate 40 (3.0 g, 69% yield) as a white solid; LCMS [M+H]: 400.4.

Compounds in table 7.4 were prepared by procedures depicted in scheme B¹¹.

TABLE 7.4

| Intermediate | Structure | m/z |
|---|---|---|
| 40.1 | 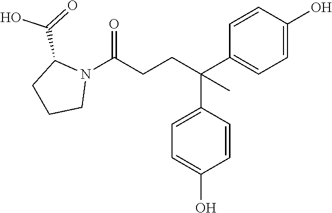 [SMILES: CC(CCC(=O)N1CCC[C@@H]1C(=O)O)(c1ccc(O)cc1)c1ccc(O)cc1] | [M + H]: 384.2 |
| 40.2 | 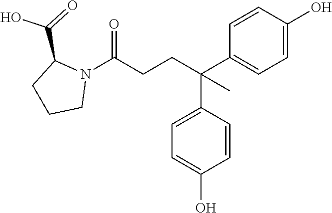 [SMILES: CC(CCC(=O)N1CCC[C@H]1C(=O)O)(c1ccc(O)cc1)c1ccc(O)cc1] | [M + H]: 384.2 |
| 40.3 | 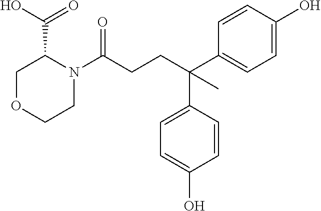 [SMILES: CC(CCC(=O)N1CCOC[C@@H]1C(=O)O)(c1ccc(O)cc1)c1ccc(O)cc1] | [M + H]: 400.2 |
| 40.4 | 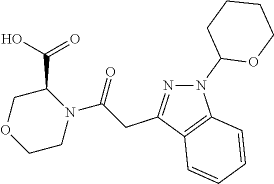 [SMILES: O=C(O)[C@@H]1COCCN1C(=O)Cc1nn(C2CCCCO2)c2ccccc12] | [M + H]: 374.2 |
| 40.5 | 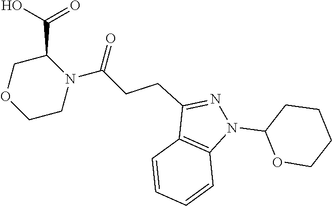 [SMILES: O=C(O)[C@@H]1COCCN1C(=O)CCc1nn(C2CCCCO2)c2ccccc12] | [M + H]: 388.3 |
| 40.6 | 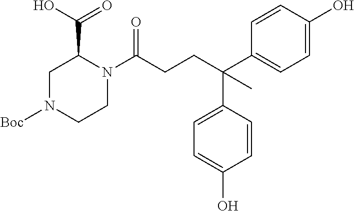 [SMILES: CC(C)(C)OC(=O)N1CCN(C(=O)CCC(C)(c2ccc(O)cc2)c2ccc(O)cc2)[C@H](C(=O)O)C1] | [M + H]: 499.3 |
| 40.7 | 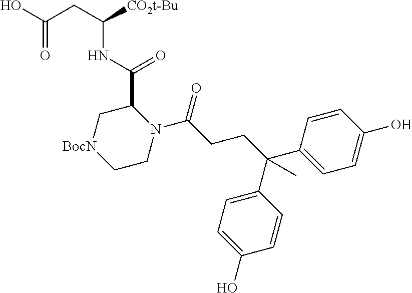 | [M + H]: 670.4 |

### B12. Synthesis of Intermediate 41

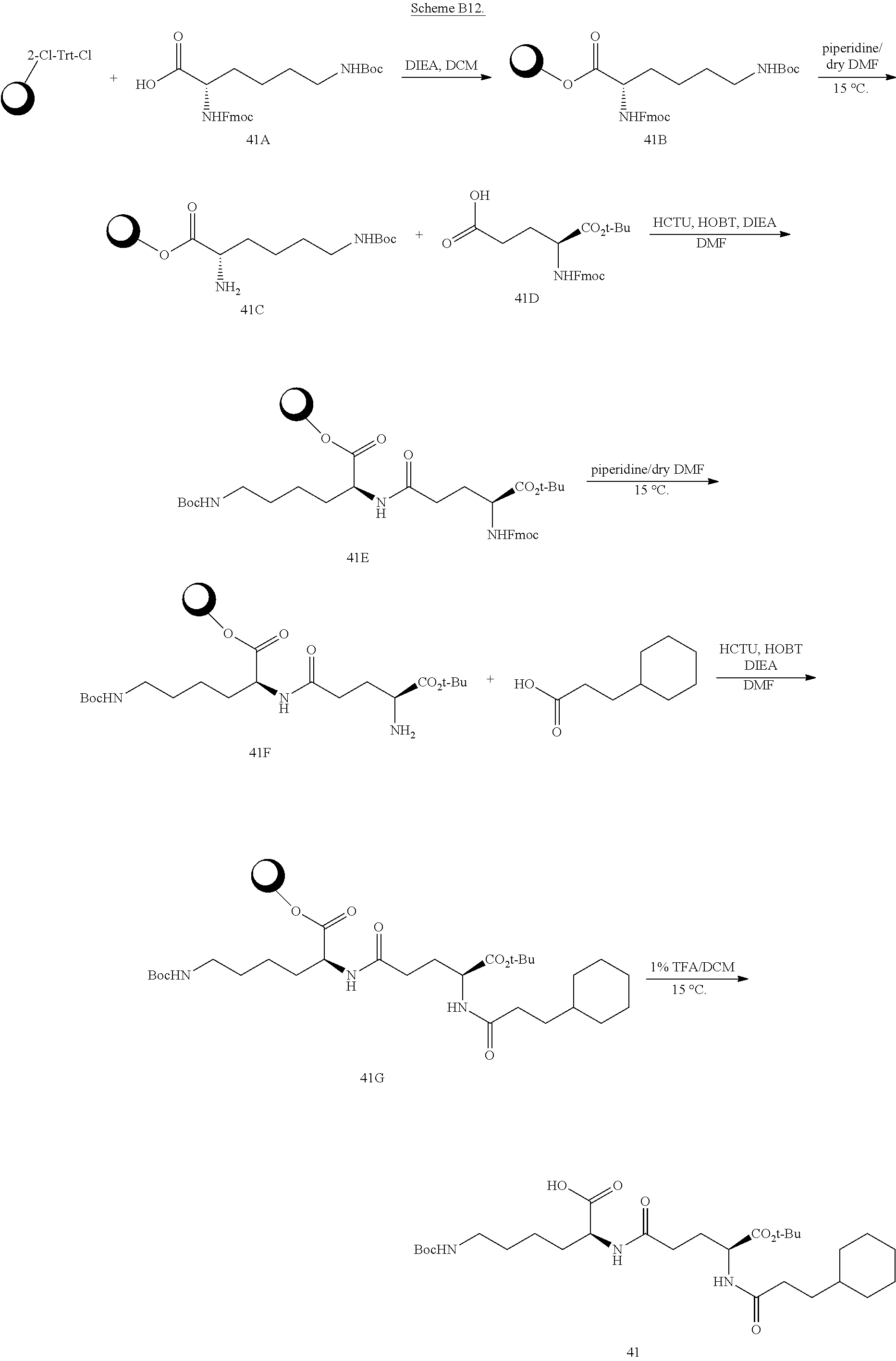

### 2.56 Synthesis of Compound 41B

To a mixture of 2-Cl-Trt-Cl resin (4.46 g, 14.2 mmol, 1 eq) in DCM (70 mL) was added a solution of compound 41A (10 g, 21.3 mmol, 1.5 eq) and DIEA (4.96 mL, 28.5 mmol, 2 eq) in DCM (70 mL) at 25° C. and the mixture shaken for 36 h under a nitrogen atmosphere. The reaction mixture was filtered, the filter cake washed with DCM (3×100 mL), DMF (2×100 mL), MeOH (3×100 mL) and DMF (2×100 mL), the resin dried with nitrogen and used directly in the next reaction.

### 2.57 Synthesis of Compound 41C

A mixture of compound 41B (20 g, 26.8 mmol, 1 eq) in piperidine (20 mL) and DMF (80 mL) was shaken at 25° C. for 10 min under a nitrogen atmosphere, the mixture filtered and the procedure repeated four times. The cake was washed with DCM (2×100 mL) and DMF (2×100 mL), the resin dried with nitrogen and used directly in the next reaction.

### 2.58 Synthesis of Compound 41E

To a mixture of compound 41D (8.54 g, 20.1 mmol, 1.5 eq) in DMF (100 mL) was added DIEA (3.96 mL, 22.8 mmol, 1.7 eq), HOBt (2.71 g, 20.1 mmol, 1.5 eq) and HCTU (8.30 g, 20.1 mmol, 1.5 eq) at 25° C. The mixture was stirred at 25° C. for 30 min, added to a solution of compound 41C (7.0 g, 13.4 mmol, 1 eq) in DMF (100 mL), and the mixture shaken at 25° C. for 1 h. The mixture was filtered, the cake washed with DMF (100 mL), DCM (3×100 mL) and DMF (2×100 mL), the resin dried with nitrogen and used directly in the next reaction.

### 2.59 Synthesis of Compound 41F

A mixture of compound 41E (14.1 g, 15.2 mmol, 1.0 eq) in piperidine (20 mL) and DMF (80 mL) was shaken at 15° C. for 10 min under a nitrogen atmosphere, and the mixture filtered. The procedure was repeated four times, the cake washed with DCM (2×100 mL) and DMF (2×100 mL), the resin dried with nitrogen and used directly in the next reaction.

### 2.60 Synthesis of Compound 41G

To a mixture of 3-cyclohexylpropanoic acid (3.81 mL, 22.2 mmol, 1.5 eq) in DMF (50 mL) was added DIEA (4.39 mL, 25.2 mmol, 1.7 eq), HOBt (3.00 g, 22.2 mmol, 1.5 eq) and HCTU (9.20 g, 22.2 mmol, 1.5 eq) at 25° C. The mixture was stirred at 25° C. for 30 min, the mixture added to a solution of compound 41F (10.5 g, 14.8 mmol, 1 eq) in DMF (50 mL), and the reaction shaken at 25° C. for 1 h. The mixture was filtered, the cake washed with DMF (100 mL), DCM (3× 100 mL) and DMF (2×100 mL), the resin dried with nitrogen and used directly in the next reaction.

### 2.61 Synthesis of Intermediate 41

To a solution of compound 41G (12 g, 14.18 mmol, 1 eq) in DCM (100 mL) was added TFA (1 mL) at 15° C. for 10 min, and the mixture filtered. This procedure was repeated three times, THF (100 mL) was added, and the reaction shaken for 10 min at 15° C. The mixture was filtered, washed with aq. NaHCO₃ (5.6 g in 50 mL H₂O), the organic layer dried (Na₂SO₄), filtered and concentrated under reduced pressure. The residue was purified by column chromatography (SiO₂, petroleum ether:EtOAc) to give Intermediate 41 (6.2 g, 77% yield) as a yellow solid; LCMS [M+H]: 570.5; ¹H NMR (400 MHz, CD₃OD) δ 4.39-4.32 (m, 1H), 4.25 (dd, J=4.9, 9.4 Hz, 1H), 3.04 (t, J=6.7 Hz, 2H), 2.37-2.31 (m, 2H), 2.30-2.21 (m, 2H), 2.20-2.05 (m, 1H), 1.95-1.81 (m, 2H), 1.80-1.62 (m, 6H), 1.61-1.49 (m, 3H), 1.47 (s, 9H), 1.43 (s, 9H), 1.40-1.35 (m, 1H), 1.34-1.23 (m, 4H), 1.23-1.13 (m, 2H), 0.98-0.86 (m, 2H).

### B13. Synthesis of Intermediate 42

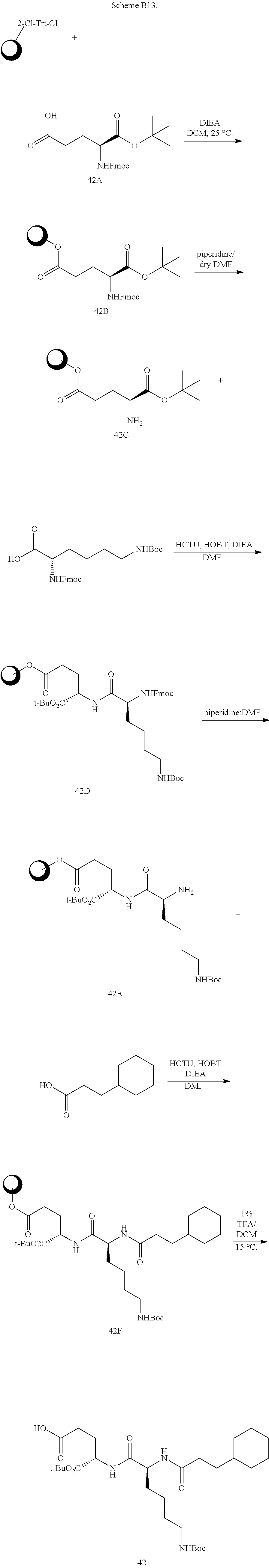

### 2.62 Synthesis of Compound 42B

To a mixture of 2-Cl-Trt-Cl resin (9.82 g, 31.3 mmol, 1.0 eq) in DCM (70 mL) was added a solution of compound 42A (20.0 g, 47.0 mmol, 1.5 eq), DIEA (10.9 mL, 62.7 mmol, 2 eq) in DCM (70 mL) at 25° C., and the mixture shaken at 25° C. for 36 h under a nitrogen atmosphere. The reaction mixture was filtered, the filter cake washed with DCM (3×100 mL), DMF (2×100 mL), MeOH (3×100 mL) and DMF (2×100 mL), the resin dried with nitrogen and used directly in the next reaction.

### 2.63 Synthesis of Compound 42C

A mixture of compound 42B (22 g, 31.3 mmol, 1.0 eq) in piperidine (20 mL) and DMF (80 mL) was shaken at 25° C. for 10 min under a nitrogen atmosphere, and the mixture filtered. The procedure was repeated four times, the cake washed with DCM (2×100 mL) and DMF (2×100 mL), the resin dried with nitrogen and used directly in the next reaction.

### 2.64 Synthesis of Compound 42D

To a mixture of N2-((9H-fluoren-9-yl)methoxy)carbonyl)-N6-(tert-butoxycarbonyl)-L-lysine (22.0 g, 46.9 mmol, 1.5 eq) in DMF (100 mL) was added DIEA (9.3 mL, 53.1 mmol, 1.7 eq), HOBt (6.33 g, 46.9 mmol, 1.5 eq) and HCTU (19.4 g, 46.9 mmol, 1.5 eq) at 25° C. The mixture was stirred at 25° C. for 30 min. The mixture was added to a solution of compound 42C (15.0 g, 31.3 mmol, 1 eq) in DMF (100 mL), and the mixture was shaken at 25° C. for 1 h. The mixture was filtered, the cake washed with DMF (100 mL), DCM (3×100 mL) and DMF (2×100 mL), the resin dried with nitrogen and used directly in the next reaction.

### 2.65 Synthesis of Compound 42E

A mixture of compound 42D (10.0 g, 10.8 mmol, 1.0 eq) in piperidine (20 mL) and DMF (80 mL) was shaken at 15° C. for 10 min under a nitrogen atmosphere, and the mixture filtered. The procedure was repeated four times, the cake was washed with DCM (2×100 mL) and DMF (2×100 mL), the resin dried with nitrogen and used directly in the next reaction.

### 2.66 Synthesis of Compound 42F

To a mixture of 3-cyclohexylpropanoic acid (2.76 mL, 16.1 mmol, 1.5 eq) in DMF (50 mL) was added DIEA (3.18 mL, 18.2 mmol, 1.7 eq), HOBt (2.17 g, 16.1 mmol, 1.5 eq) and HCTU (6.66 g, 16.1 mmol, 1.5 eq) at 25° C. The mixture was stirred at 25° C. for 30 min, added to a solution of compound 42E (7.6 g, 10.7 mmol, 1 eq) in DMF (50 mL), and the mixture shaken at 25° C. for 1 h. The mixture was filtered, the cake washed with DMF (100 mL), DCM (3×100 mL) and DMF (2×100 mL), the resin dried with nitrogen and used directly in the next reaction.

### 2.67 Synthesis of Intermediate 42

To a solution of compound 42F (9.0 g, 10.6 mmol, 1 eq) in DCM (100 mL) was added TFA (1 mL) at 15° C. for 10 min and the mixture filtered. The procedure was repeated three times, THF (100 mL) was added, the reaction shaken for 10 min at 15° C. and the mixture filtered. The mixture was washed with aq. NaHCO₃ (3.4 g in 50 mL H₂O), the organic layer dried (Na₂SO₄), filtered and concentrated under reduced pressure. The residue was purified by column chromatography (SiO₂, petroleum ether:EtOAc) to give Intermediate 42 (6.0 g, 99% yield) as a yellow oil; LCMS [M+H]: 570.5; ¹H NMR (400 MHz, CHLOROFORM-d) δ 7.45-7.31 (m, 1H), 6.45 (br d, J=2.9 Hz, 1H), 4.83-4.68 (m, 1H), 4.57-4.37 (m, 2H), 3.20-3.07 (m, 2H), 2.52-2.36 (m, 2H), 2.29-2.17 (m, 3H), 2.12-1.97 (m, 1H), 1.89-1.76 (m, 2H), 1.74-1.59 (m, 6H), 1.55-1.49 (m, 3H), 1.48-1.42 (m, 18H), 1.30 (br d, J=8.7 Hz, 5H), 0.97-0.80 (m, 2H).

### B14. Synthesis of Intermediate 43

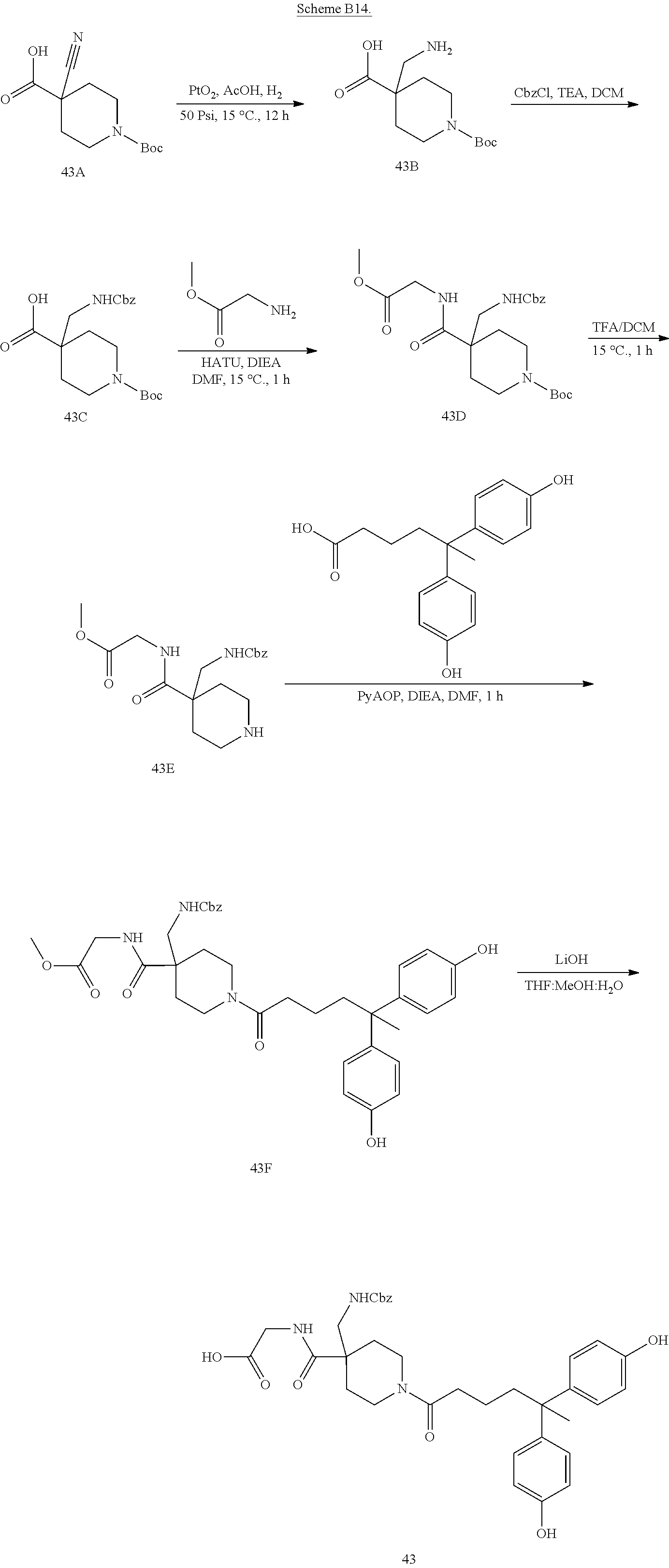

### 2.68 Synthesis of Compound 43B

To a solution of compound 43A (2.8 g, 11.0 mmol, 1 eq) in AcOH (20 mL) was added PtO₂ (300 mg, 1.32 mmol, 0.12 eq). The mixture was stirred at 15° C. for 12 h under a hydrogen atmosphere (50 Psi). The reaction mixture was filtered and the filtrate concentrated under reduced pressure to give compound 43B (2.8 g, 98% yield) as a colorless oil; LCMS [M+Na]: 281.2.

### 2.69 Synthesis of Compound 43C

To a solution of compound 43B (3.8 g, 14.7 mmol, 1 eq) in DCM (40 mL) was added DIEA (3.07 mL, 17.7 mmol, 1.2 eq) and CbzCl (2.21 mL, 15.5 mmol, 1.05 eq) at 0° C. The mixture was stirred at 15° C. for 1h, concentrated under reduced pressure and the residue purified by column chromatography (SiO₂, DCM/MeOH) to give compound 43C (3.5 g, 61% yield) as a colorless oil; LCMS [M−100+1]: 293.3; ¹H NMR (400 MHz, CHLOROFORM-d) δ 7.29 (s, 5H), 5.15-4.95 (m, 2H), 4.61 (s, 7H), 4.04 (q, J=7.1 Hz, 2H), 3.74-3.58 (m, 1H), 3.06-2.91 (m, 2H), 2.57 (d, J=10.1 Hz, 1H), 1.66-1.52 (m, 2H), 1.44-1.33 (m, 9H).

### 2.70 Synthesis of Compound 43D

To a solution of compound 43C (3.0 g, 7.64 mmol, 1 eq) and methyl glycinate (960 mg, 7.64 mmol, 1 eq, HCl) in DMF (30 mL) was added DIEA (3.99 mL, 22.9 mmol, 3 eq) and HATU (2.91 g, 7.64 mmol, 1 eq) at 15° C. The reaction mixture was stirred at 15° C. for 1 h, H₂O (50 mL) added and extracted with EtOAc (2×50 mL). The combined organic phase was dried (Na₂SO₄), concentrated under reduced pressure and the residue purified by column chromatography (SiO₂, petroleum ether:EtOAc) to give compound 43D (5.0 g) as a yellow solid; LCMS [M−100+H]: 364.2; ¹H NMR (400 MHz, CHLOROFORM-d) δ 7.43-7.31 (m, 5H), 6.15 (t, J=5.6 Hz, 1H), 5.74-5.65 (m, 1H), 5.11 (s, 2H), 4.04 (d, J=5.1 Hz, 2H), 3.82-3.74 (m, 5H), 3.67 (d, J=12.4 Hz, 2H), 3.40 (br s, 2H), 3.25 (t, J=9.8 Hz, 2H), 1.96-1.88 (m, 2H), 1.45 (s, 9H).

### 2.71 Synthesis of Compound 43E

To a solution of compound 43D (2.2 g, 4.75 mmol, 1 eq) in DCM (6 mL) was added TFA (2 mL) stirred at 15° C. The mixture was stirred at 15° C. for 1 h, H₂O (20 mL) added and extracted with MTBE (3×10 mL). The water phase was adjusted to pH=11 with aq. K₂CO₃ and extracted with DCM (3×30 mL). The combined organic phase was dried (Na₂SO₄) and concentrated under reduced pressure to give compound 43E (1.2 g, 70% yield) as a white solid; LCMS [M+H]: 364.1.

### 2.72 Synthesis of Compound 43F

To a solution of compound 43E (3.0 g, 8.32 mmol, 1 eq) and 5,5-bis(4-hydroxyphenyl)hexanoic acid (2.5 g, 8.32 mmol, 1 eq) in DMF (10 mL) was added DIEA (3.23 g, 24.97 mmol, 4.35 mL, 3 eq) and PyAOP (4.55 g, 8.73 mmol, 1.05 eq) at 15° C. The mixture was stirred at 15° C. for 1 h, diluted with water (50 mL) and extracted with EtOAc (3×50 mL). The combined organic layers were washed with brine (30 mL), dried (Na₂SO₄), filtered and concentrated under reduced pressure. The residue was purified by column chromatography (SiO₂, petroleum ether:EtOAc and DCM:MeOH) to give compound 43E (6.0 g, 81% yield, 72% purity) as a yellow oil; LCMS [M+1]: 646.4.

### 2.73 Synthesis of Intermediate 43

To a solution of compound 43E (6.5 g, 10.07 mmol, 1 eq) in THF (5 mL), MeOH (5 mL) and H₂O (5 mL) was added LiOH (1.21 g, 50.3 mmol, 5 eq) at 15° C. The mixture was stirred at 15° C. for 3 h, H₂O (10 mL) added, and the mixture adjusted to pH=2 with 2N HCl (~15 mL). The suspension was filtered and the filter cake concentrated under reduced pressure to give Intermediate 43 (5.0 g, 79% yield) as a white solid; LCMS [M+H]: 632.4.

### B15. Synthesis of Intermediate 44

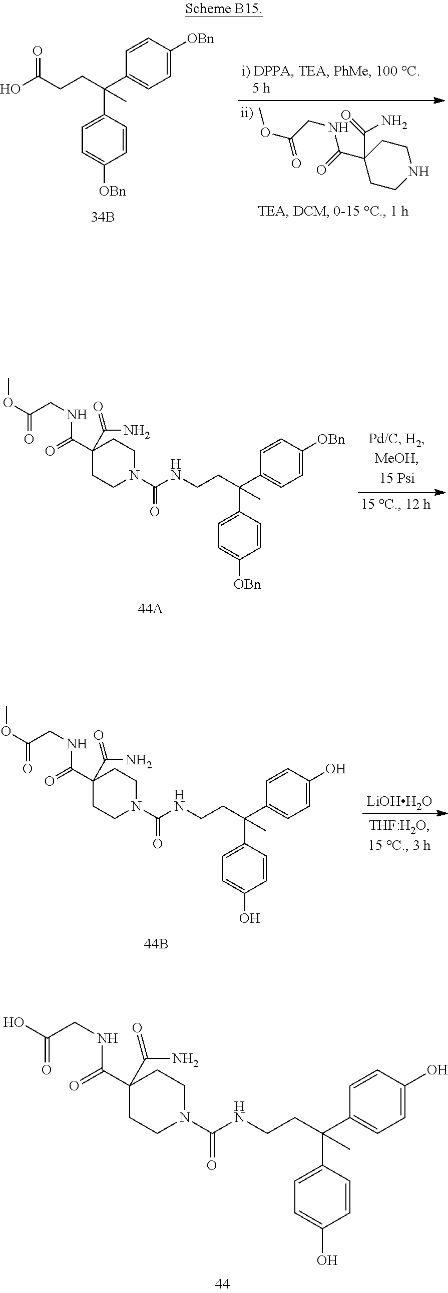 [SMILES: CC(CCC(=O)O)(c1ccc(OCc2ccccc2)cc1)c1ccc(OCc2ccccc2)cc1 ; COC(=O)CNC(=O)C1(C(N)=O)CCNCC1 ; COC(=O)CNC(=O)C1(C(N)=O)CCN(C(=O)NCCC(C)(c2ccc(OCc3ccccc3)cc2)c2ccc(OCc3ccccc3)cc2)CC1 ; COC(=O)CNC(=O)C1(C(N)=O)CCN(C(=O)NCCC(C)(c2ccc(O)cc2)c2ccc(O)cc2)CC1 ; "LiOH·H2O" ; CC(CCNC(=O)N1CCC(C(N)=O)(C(=O)NCC(=O)O)CC1)(c1ccc(O)cc1)c1ccc(O)cc1]

### 2.76 Synthesis of Compound 44A

To a solution of compound 34B (3.0 g, 6.42 mmol, 1 eq) in toluene (45 mL) was added TEA (3.58 mL, 25.7 mmol, 4 eq) and DPPA (1.52 mL, 7.08 mmol, 1.1 eq). The mixture was stirred at 100° C. for 5 h, methyl (4-carbamoylpiperidine-4-carbonyl)glycinate (1.95 g, 5.46 mmol, 0.85 eq, TFA salt) and TEA (2.98 mL, 21.4 mmol) in DCM (20 mL) added at 0° C., and the reaction stirred at 0° C. for 1 h under a nitrogen atmosphere. The reaction mixture was concentrated under reduced pressure and the residue purified by column chromatography (SiO₂, Petroleum ether:EtOAc) to give compound 44A (3.03 g, 67% yield) as a white solid; LCMS [M+H]: 707.5.

### 2.77 Synthesis of Compound 44B

To a solution of compound 44A (4.5 g, 6.36 mmol, 1 eq) in MeOH (50 mL) was added Pd/C (4.0 g, 10% purity). The suspension was degassed and purged with hydrogen 3 times. The mixture was stirred under a hydrogen atmosphere (15 Psi) at 15° C. for 12 h, filtered and the filtrate concentrated under reduced pressure to give crude compound 44B (3.35 g, crude) as a white solid; LCMS [M+H]: 527.4.

### 2.78 Synthesis of Intermediate 44

To a solution of compound 44B (3.36 g, 3.19 mmol, 1 eq) in THF (15 mL) and H₂O (15 mL) was added LiOH. H₂O (803 mg, 19.1 mmol, 3 eq). The mixture was stirred at 15° C. for 3 h and concentrated under reduced pressure to remove the THF. The mixture was adjusted to pH=2 with 2M HCl. The mixture was concentrated under reduced pressure to give intermediate 44 (3.27 g, crude) as a yellow solid; LCMS [M+H]: 513.3.

### B16. Synthesis of Intermediate 45

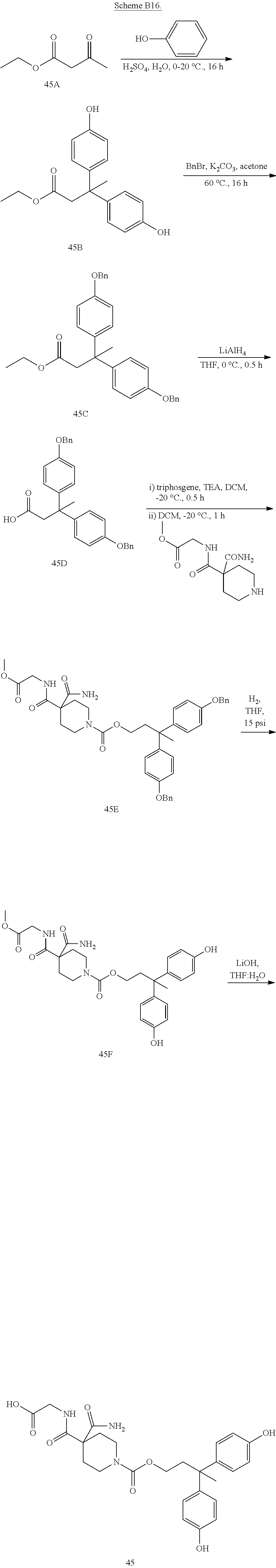

### 2.79 Synthesis of Compound 45B

To a solution of compound 45A (9.71 mL, 76.8 mmol, 1 eq), phenol (13.5 mL, 154 mmol, 2 eq) in H₂O (3.46 mL, 192 mmol, 2.5 eq) was added H₂SO₄ (7.54 mL, 141 mmol, 1.84 eq) dropwise at 0° C. for 15 min. The mixture was stirred at 20° C. for 16 h, diluted with H₂O (300 mL) and extracted with EtOAc (300 mL). The organic layer was washed with NaHCO₃ (2×200 mL) and H₂O (200 mL), the organic layer dried (Na₂SO₄), filtered and concentrated under reduced pressure. The residue was purified by column chromatography (SiO₂, Petroleum ether:EtOAc) to give compound 45B (17.4 g, 38% yield) as a colorless oil; ¹H NMR (400 MHz, CHLOROFORM-d) δ 7.05 (d, J=8.6 Hz, 4H), 6.71 (d, J=8.6 Hz, 4H), 4.98 (s, 2H), 3.91 (q, J=7.2 Hz, 2H), 3.07 (s, 2H), 1.81 (s, 3H), 1.03 (t, J=7.2 Hz, 3H).

### 2.80 Synthesis of Compound 45C

To a solution of compound 45B (12 g, 40.0 mmol, 1 eq), BnBr (47.5 mL, 400 mmol, 10 eq) and K₂CO₃ (55 g, 400 mmol, 10 eq) in acetone (120 mL) was degassed and purged with nitrogen 3 times, and stirred at 60° C. for 16 h under a nitrogen atmosphere. The reaction mixture was diluted with H₂O (50 mL) and extracted with EtOAc (3×50 mL). The combined organic layers were washed with NaHCO₃ (3×50 m), dried (Na₂SO₄), filtered and concentrated under reduced pressure. The residue was purified by column chromatography (SiO₂, Petroleum ether:EtOAc) to give compound 45C (9.0 g, 58% yield) as a colorless oil; ¹H NMR (400 MHz, CHLOROFORM-d) δ 7.41-7.27 (m, 8H), 7.27-7.16 (m, 2H), 7.08-7.01 (m, 4H), 6.87-6.78 (m, 4H), 4.97 (s, 4H), 3.82 (q, J=7.2 Hz, 2H), 3.00 (s, 2H), 1.76 (s, 3H), 0.92 (t, J=7.2 Hz, 3H).

### 2.81 Synthesis of Compound 45D

To a solution of compound 45C (6.0 g, 15.4 mmol, 1 eq) in THF (60 mL) was added 2.5 M LiAlH₄ (12.3 mL, 2 eq) at 0° C. under a nitrogen atmosphere. The mixture was stirred at 0° C. for 0.5 h, quenched with H₂O (30 mL) and stirred at 20° C. for 30 min. The mixture was extracted with EtOAc (2×50 mL), the combined organic phase washed with brine (2×50 mL), dried (Na₂SO₄), filtered and concentrated under reduced pressure. The residue was purified by column chromatography (SiO₂, Petroleum ether:EtOAc) to give compound 45D (5.0 g, 93% yield) as a white solid; ¹H NMR (400 MHz, CHLOROFORM-d) δ 7.43-7.15 (m, 10H), 7.05 (d, J=8.8 Hz, 4H), 6.82 (d, J=8.8 Hz, 4H), 4.96 (s, 4H), 3.46 (t, J=7.4 Hz, 2H), 2.32 (t, J=7.4 Hz, 2H), 1.55 (s, 3H).

### 2.82 Synthesis of Compound 45E

To a solution of compound 45D (4.0 g, 11.5 mmol, 1 eq) in DCM (60 mL) was added TEA (6.39 mL, 45.9 mmol, 4 eq) and triphosgene (2.04 g, 6.89 mmol, 0.6 eq) at −20° C., and the mixture stirred for 0.5 h. Methyl (4-carbamoylpiperidine-4-carbonyl)glycinate (4.10 g, 11.48 mmol, 1 eq, TFA) was added at −20° C. and the reaction stirred at −20° C. for 1 h. H₂O (50 mL) was added, and the reaction extracted with DCM (3×30 mL). The combined organic phase was dried (Na₂SO₄) and concentrated under reduced pressure. The residue was purified by column (SiO₂, EtOAc to DCM;MeOH) to give compound 45E (5.0 g, 83% yield) as a white solid; LCMS [M+1]: 708.5; ¹H NMR (400 MHz, CHLOROFORM-d) δ 7.42-7.21 (m, 10H), 7.08-7.01 (m, 4H), 6.84-6.77 (m, 4H), 4.99-4.92 (m, 4H), 3.94-3.84 (m, 4H), 3.70-3.66 (m, 3H), 3.53 (ddd, J=3.8, 8.2, 17.5 Hz, 2H), 3.38-3.19 (m, 2H), 2.35 (t, J=7.3 Hz, 2H), 1.97 (br s, 2H), 1.57 (s, 3H).

### 2.83 Synthesis of Compound 45F

To a solution of compound 45E (4.5 g, 6.36 mmol, 1 eq) in THF (60 mL) was added Pd/C (0.7 g, 658 μmol, 10% purity). The mixture was stirred at 20° C. for 5 h under a hydrogen atmosphere (15 psi), filtered and the filtrate concentrated under reduced pressure. The residue was purified by column chromatography (SiO₂, petroleum ether::EtOAc to DCM/MeOH) to give compound 45F (3.3 g, 98% yield) as a white solid; LCMS [M+H]: 528.3; ¹H NMR (400 MHz, DMSO-d₆) δ 9.18 (s, 2H), 8.21-8.09 (m, 1H), 6.96 (d, J=8.5 Hz, 4H), 6.65 (d, J=8.5 Hz, 4H), 3.83 (d, J=5.5 Hz, 2H), 3.67-3.60 (m, 5H), 3.43 (dd, J=5.5, 9.0 Hz, 2H), 3.17 (t, J=10.3 Hz, 2H), 2.31 (t, J=7.3 Hz, 2H), 2.06-1.94 (m, 2H), 1.88-1.78 (m, 2H), 1.52 (s, 3H).

### 2.84 Synthesis of Compound 45

To a solution of compound 45F (3.0 g, 5.69 mmol, 1 eq) in THF (5 mL) and H₂O (5 mL) was added LiOH (545 mg, 22.8 mmol, 4 eq) at 15° C. The mixture was stirred at 15° C. for 3 h, concentrated under reduced pressure, H₂O (5 mL) added to the residue and 2N HCl (~5 mL) added to pH=2. The suspension was filtered and concentrated under reduced pressure to give Intermediate 45 (2.9 g, 99% yield) as a white solid; LCMS [M+H]: 514.3.

### B17. Synthesis of Intermediate 46

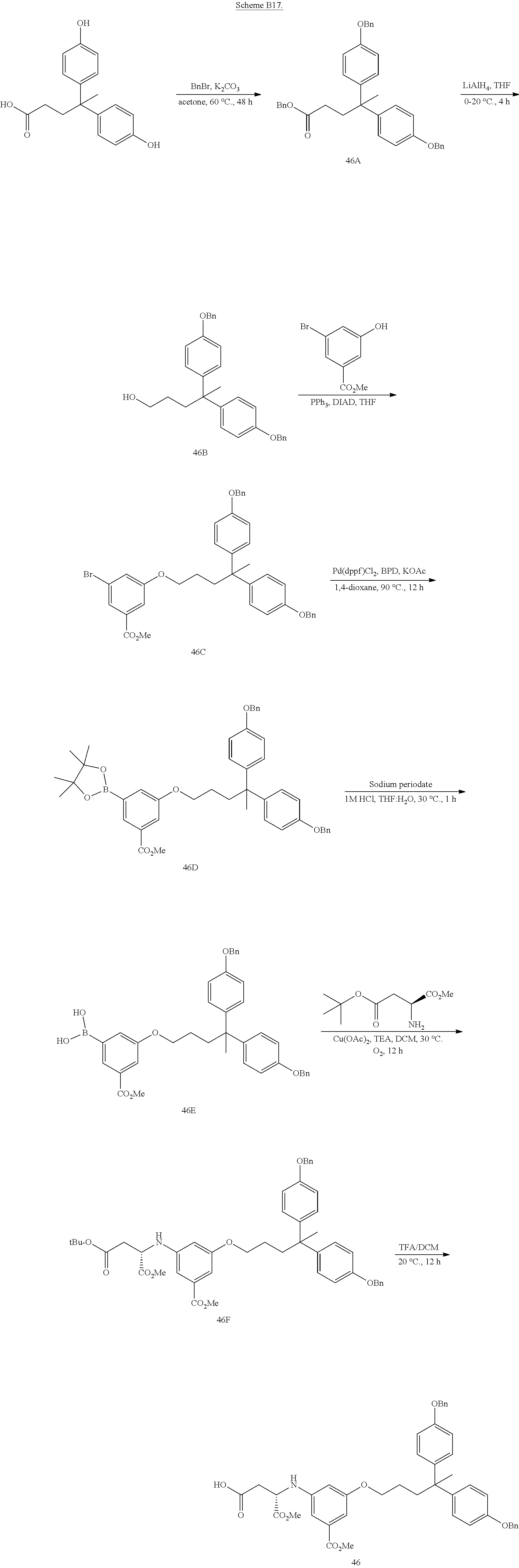

### 2.85 Synthesis of Compound 46A

To a mixture of 4,4-bis(4-hydroxyphenyl)pentanoic acid (10.0 g, 34.9 mmol, 1 eq) and K₂CO₃ (24.1 g, 174.6 mmol, 5.0 eq) in acetone (100 mL) was added BnBr (20.7 mL, 175 mmol, 5.0 eq) at 20° C. The mixture was stirred at 60° C. for 48 h, filtered and concentrated under reduced pressure. The residue was diluted with EtOAc (100 mL), the organic layer washed with H₂O (30 mL) and brine (30 mL), dried (Na₂SO₄), filtered and concentrated under reduced pressure. The residue was purified by column chromatography (SiO₂, Petroleum ether:EtOAc) to give compound 46A (15.5 g, 80% yield) as a white solid; ¹H NMR (400 MHz, CDCl₃-d) δ 7.46-7.30 (m, 15H), 7.10 (s, 4H), 6.89 (d, J=8.8 Hz, 4H), 5.07 (s, 2H), 5.03 (s, 4H), 2.48-2.41 (m, 2H), 2.21-2.14 (m, 2H), 1.58 (s, 3H).

### 2.86 Synthesis of Compound 46B

A mixture of LiAlH₄ (2.8 g, 75.1 mmol, 4.0 eq) in THF (50 mL) was stirred at 0° C. for 10 minutes under a nitrogen atmosphere. Compound 46A (10.4 g, 18.7 mmol, 1.0 eq) in THF (50 mL) was added dropwise, the temperature warmed to 20° C. and stirred for 4 h. The reaction mixture was quenched by addition of H₂O (2.8 mL), 15% NaOH (2.8 mL) and H₂O (8.0 mL) at 0° C. The mixture was filtered and concentrated under reduced pressure. The residue was purified by column chromatography (SiO₂, Petroleum ether:EtOAc) to give compound 46B (8.2 g, 97% yield) as a white solid; ¹H NMR (400 MHz, CDCl₃-d) δ 7.51-7.32 (m, 10H), 7.14 (d, J=8.8 Hz, 4H), 6.90 (d, J=8.8 Hz, 4H), 5.05 (s, 4H), 3.61 (t, J=6.6 Hz, 2H), 2.17-2.09 (m, 2H), 1.62 (s, 3H), 1.44-1.34 (m, 2H)

### 2.87 Synthesis of Compound 46C

To a solution of compound 46B (6.0 g, 13.2 mmol, 1.0 eq) and methyl 3-bromo-5-hydroxybenzoate (3.1 g, 13.2 mmol, 1.0 eq) in THF (60 mL) was added PPh₃ (5.2 g, 19.9 mmol, 1.5 eq) at 15° C. The mixture was degassed and purged with nitrogen 3 times. DIAD (4.0 g, 19.8 mmol, 3.8 mL, 1.5 eq) was added dropwise at 0° C., the mixture stirred at 0° C. for 1 h under a nitrogen atmosphere and concentrated under reduced pressure. The residue was diluted with EtOAc (100 mL), the organic layer washed with NaHCO₃ (30 mL), dried (Na₂SO₄), filtered and concentrated under reduced pressure. The residue was purified by column chromatography (SiO₂, Petroleum ether:EtOAc) to give compound 46C (7.1 g, 81% yield) as a yellow oil; ¹H NMR (400 MHz, CDCl₃-d) δ 7.75 (s, 1H), 7.49-7.31 (m, 11H), 7.22-7.19 (m, 1H), 7.14 (d, J=8.8 Hz, 4H), 6.90 (d, J=8.8 Hz, 4H), 5.05 (s, 4H), 3.96-3.89 (m, 5H), 2.26-2.18 (m, 2H), 1.64 (m, 5H).

### 2.88 Synthesis of Compound 46D

To a solution of compound 46C (5.7 g, 8.5 mmol, 1.0 eq) in 1,4-dioxane (57 mL) was added BPD (5.4 g, 21.4 mmol, 2.5 eq), KOAc (2.9 g, 29.9 mmol, 3.5 eq) and Pd(dppf)Cl₂ (627 mg, 856 μmol, 0.1 eq). The mixture was degassed and purged with nitrogen 3 times, stirred at 90° C. for 12 h under a nitrogen atmosphere and concentrated under reduced pressure. The residue was diluted with EtOAc (60 mL), the organic layer washed with H₂O (20 mL), brine (20 mL), dried (Na₂SO₄), filtered, concentrated under reduced pressure and purified by column chromatography (SiO₂, Petroleum ether:EtOAc) to give crude compound 46D (7.6 g) as a colorless oil; ¹H NMR (400 MHz, CDCl₃-d) δ 8.05 (s, 1H), 7.62 (dd, J=1.6, 2.4 Hz, 1H), 7.50 (d, J=2.6 Hz, 1H), 7.47-7.31 (m, 10H), 7.14 (d, J=8.8 Hz, 4H), 6.89 (d, J=8.8 Hz, 4H), 5.04 (s, 4H), 4.00-3.95 (m, 2H), 3.91 (s, 3H), 2.26-2.20 (m, 2H), 1.66-1.57 (m, 5H), 1.36 (s, 12H).

### 2.89 Synthesis of Compound 46E

To a solution of compound 46D (720 mg, 1.0 mmol, 1.0 eq) and sodium periodate (97.9 μL, 1.7 mmol, 1.7 eq) in THF (32 mL) and H₂O (8 mL) was added 1M HCl (5.7 mL, 5.7 eq). The mixture was stirred at 30° C. for 1 h, diluted with brine (20 mL), and extracted with EtOAc (2×20 mL). The combined organic layers were washed with Na₂SO₃ (20 mL), dried (Na₂SO₄), filtered and concentrated under reduced pressure to give a crude compound 46E (669 mg) as a white solid; LCMS [M−H]: 629.2; ¹H NMR (400 MHz, CDCl₃-d) δ 8.43 (s, 1H), 7.90 (d, J=2.0 Hz, 1H), 7.75 (d, J=1.0 Hz, 1H), 7.46-7.29 (m, 10H), 7.19-7.12 (m, 4H), 6.92-6.86 (m, 4H), 5.06-4.99 (m, 4H), 4.10-4.01 (m, 2H), 3.98-3.92 (m, 3H), 2.32-2.20 (m, 2H), 1.74-1.61 (m, 5H).

### 2.90 Synthesis of Compound 46F

To a solution of compound 46E (2.1 g, 3.3 mmol, 1.0 eq) and 4-(tert-butyl) 1-methyl L-aspartate (1.3 g, 6.6 mmol, 2.0 eq) in DCM (21 mL) were added 4 Å molecular sieves (2.1 g), TEA (464 μL, 3.3 mmol, 1.0 eq) and Cu(OAc)₂ (3.0 g, 16.6 mmol, 5.0 eq) at 30° C. The mixture was degassed and purged with oxygen 3 times and stirred at 30° C. for 12 h under an oxygen atmosphere. The reaction mixture was diluted with DCM (80 mL), filtered, the combined organic layers washed with NaHCO₃ (20 mL), dried (Na₂SO₄), filtered and concentrated under reduced pressure. The residue was purified by column chromatography (SiO₂, Petroleum ether:EtOAc) to give crude compound 46F (2.1 g) as a yellow solid; LCMS [M+H]: 788.5; ¹H NMR (400 MHz, CDCl₃-d) δ 7.45-7.33 (m, 10H), 7.14 (br d, J=8.8 Hz, 4H), 6.94 (d, J=2.0 Hz, 2H), 6.89 (br d, J=8.8 Hz, 4H), 6.36 (s, 1H), 5.05-5.02 (m, 4H), 4.43-4.38 (m, 1H), 3.92-3.87 (m, 5H), 3.76 (s, 3H), 2.87-2.75 (m, 2H), 2.25-2.17 (m, 2H), 1.66-1.59 (m, 5H), 1.44 (s, 9H)

### 2.91 Synthesis of Compound 46

A mixture of compound 46F (1.9 g, 2.4 mmol, 1.0 eq) in TFA (9 mL) and DCM (45 mL) was stirred at 20° C. for 12 h under a nitrogen atmosphere. The reaction mixture was concentrated under reduced pressure and the residue purified by column chromatography (SiO₂, Petroleum ether:EtOAc) to give crude compound 46 (1.9 g) as a yellow oil; LCMS [M+H]: 732.4; ¹H NMR (400 MHz, CDCl₃-d) 8 7.46-7.31 (m, 10H), 7.13 (br d, J=8.8 Hz, 4H), 6.96 (br s, 2H), 6.89 (br d, J=8.8 Hz, 4H), 6.37 (br s, 1H), 5.03 (s, 4H), 4.46 (brt, J=5.0 Hz, 1H), 3.93-3.86 (m, 5H), 3.77 (s, 3H), 2.99-2.93 (m, 2H), 2.23-2.17 (m, 2H), 1.65-1.56 (m, 5H).

### B18. Synthesis of Intermediate 47

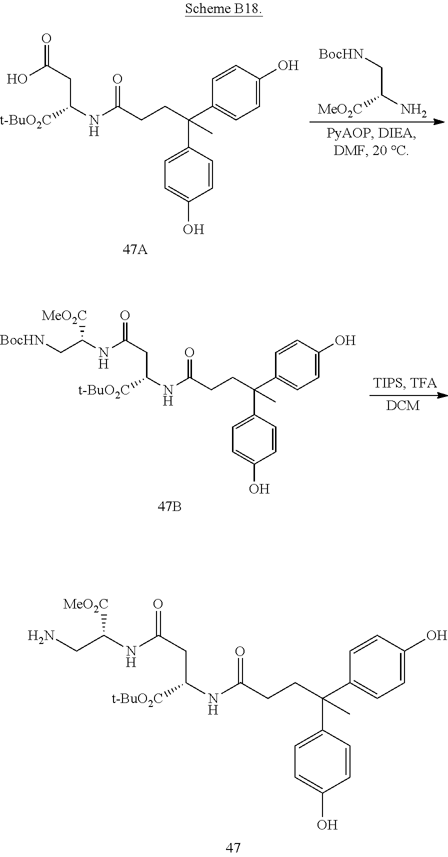

### 2.92 Synthesis of Compound 47B

To a mixture of methyl(S)-2-amino-3-((tert-butoxycarbonyl)amino)propanoate (2.0 g, 9.16 mmol, 1 eq) and compound 47A (4.19 g, 9.16 mmol, 1 eq) in DMF (20 mL) was added DIEA (3.19 mL, 18.3 mmol, 2 eq) and PYAOP (5.26 g, 10.1 mmol, 1.1 eq) in one portion at 20° C. under a nitrogen atmosphere. The mixture was stirred at 20° C. for 1 h, poured into water (50 mL) and extracted with EtOAc (3×100 mL). The combined organic phase was washed with brine (2×100 mL), dried (Na₂SO₄), filtered and concentrated under reduced pressure. The crude product was purified by column chromatography (SiO₂, DCM:MeOH) to give compound 47B (3.4 g, 51% yield, 90% purity) as light yellow solid; LCMS [M+H]: 658.4; ¹H NMR (400 MHz, DMSO-d₆) δ 9.14 (s, 2H), 6.97-6.88 (m, J=8.8 Hz, 4H), 6.64 (d, J=8.6 Hz, 4H), 4.39 (br d, J=6.4 Hz, 1H), 4.32-4.22 (m, 1H), 3.72 (s, 3H), 3.23 (br t, J=6.0 Hz, 2H), 2.61-2.51 (m, 1H), 2.47-2.41 (m, 1H), 2.23-2.10 (m, 2H), 1.91-1.79 (m, 2H), 1.45 (s, 3H), 1.35 (s, 18H)

### 2.93 Synthesis of Compound 47

To a mixture of compound 47B (2.4 g, 3.65 mmol, 1 eq) in DCM (60 mL) was added triisopropylsilane (15.0 mL, 73 mmol, 20 eq) and TFA (1.90 mL, 25.5 mmol, 7 eq) in one portion at 20° C. under a nitrogen atmosphere. The mixture was stirred at 20° C. for 16 h, poured into water (20 mL) and the aqueous phase extracted with ethyl acetate (3×50 mL). The combined organic phase was washed with NaHCO₃ (3×30 mL), brine (2×30 mL), dried (Na₂SO₄), filtered and concentrated under reduced pressure. The residue was purified by column chromatography (SiO₂, Petroleum ether:EtOAc) to give Intermediate 47 (800 mg, 39% yield) as a light yellow solid; LCMS [M+H]: 558.4; ¹H NMR (400 MHz, METHANOL-d₄) δ 7.05-6.97 (m, 4H), 6.72-6.64 (m, 4H), 4.59 (dd, J=5.6, 6.8 Hz, 1H), 4.45 (dd, J=4.8, 7.2 Hz, 1H), 3.74-3.69 (m, 3H), 3.09-2.97 (m, 1H), 2.95-2.85 (m, 1H), 2.73 (dd, J=6.2, 7.8 Hz, 2H), 2.40-2.32 (m, 2H), 2.06-2.02 (m, 2H), 1.55 (s, 3H), 1.45 (s, 9H).

### B18. Synthesis of Intermediate 48

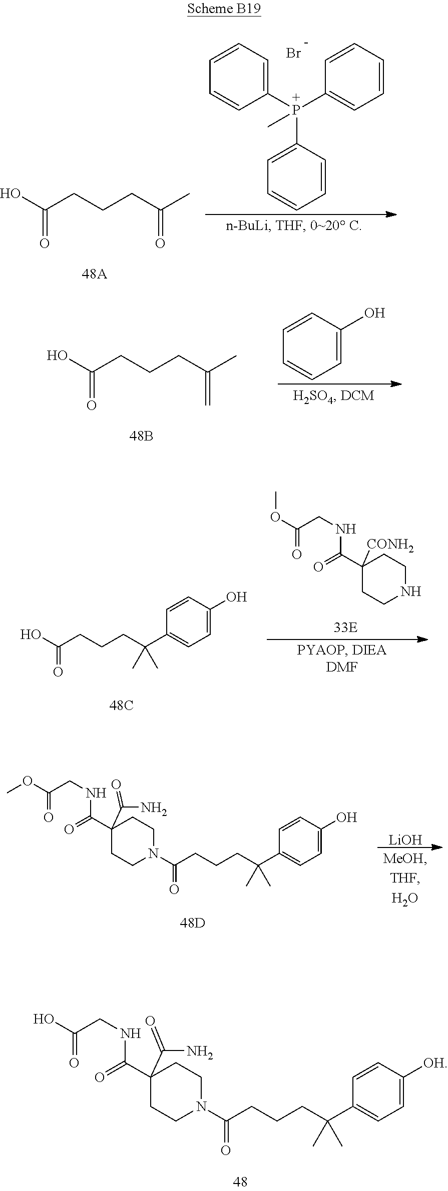

### 2.94 Synthesis of Compound 48B

To a solution of methyltriphenylphosphonium bromide (247 g, 692 mmol) in THF (600 mL) was added 2.5 M n-BuLi (277 mL, 693 mmol) at 0° C. The mixture was stirred at 0° C. for 1 h, compound 48A (28 mL, 231 mmol) in THF (100 mL) was added dropwise and the reaction stirred at 20° C. for 12 h. The reaction mixture was quenched by addition of saturated NH₄Cl solution (200 mL) at 0° C., diluted with H₂O (500 mL) and extracted with EtOAc (3×500 mL). The combined organic layers were washed with brine (3×500 mL), dried (Na₂SO₄), filtered and concentrated under reduced pressure. The residue was purified by column chromatography (SiO₂, Petroleum ether:EtOAc) to give compound 48B (25 g, 95%) as a white oil; ¹H NMR (400 MHz, CDCl₃) δ 11.72 (br s, 1H), 4.73 (br d, J=19.9 Hz, 2H), 2.36 (t, J=7.5 Hz, 2H), 2.11-2.04 (m, 2H), 1.79 (quin, J=7.5 Hz, 2H), 1.73 (s, 3H).

### 2.95 Synthesis of Compound 48C

To a solution of compound 48B (5.0 g, 39.0 mmol) and phenol (17.1 mL, 195 mmol) in DCM (150 mL) was added H₂SO₄ (10.4 mL, 195 mmol) at −20° C. The mixture was stirred at 0° C. for 0.5 h and concentrated under reduced pressure. The residue was purified by column chromatography (SiO₂, Petroleum ether:EtOAc) to give compound 48C (1.5 g, 17% yield) as a white solid; LCMS: [M−1]⁺=221.3; ¹H NMR (400 MHz, DMSO-d₆) δ 11.92 (br s, 1H) 9.10 (s, 1H) 7.10 (d, J=8.8 Hz, 2H) 6.67 (d, J=8.6 Hz, 2H) 2.08 (t, J=7.4 Hz, 2H) 1.46-1.57 (m, 2H) 1.22 (br d, J=3.8 Hz, 2H) 1.19 (s, 6H).

### 2.96 Synthesis of Compound 48D

To a solution of Compound 48C (1.35 g, 6.07 mmol) and Compound 33E (1.70 g, 6.98 mmol, 1.15 eq) in DMF (20 mL) was added DIEA (3.17 mL, 18.2 mmol) and PyAOP (3.80 g, 7.29 mmol, 1.2 eq) at 20° C. The mixture was stirred at 20° C. for 1 h. The reaction mixture was diluted with water (20 mL) and extracted with EtOAc (3×15 mL). The combined organic layers were washed with brine (10 mL), dried (Na₂SO₄), filtered and concentrated under reduced pressure. The residue was purified by column chromatography (SiO₂, DCM:MeOH) to give Compound 48D (1.5 g, 55% yield) as a white solid; LCMS [M+H]⁺=448.5; ¹H NMR (400 MHz, DMSO-d₆) δ 9.10 (s, 1H) 8.15 (t, J=5.8 Hz, 1H) 7.23 (s, 1H) 7.11 (d, J=8.6 Hz, 2H) 7.07 (s, 1H) 6.67 (d, J=8.6 Hz, 2H) 3.83 (d, J=5.8 Hz, 2H) 3.62 (s, 4H) 3.38-3.49 (m, 1H) 3.21-3.31 (m, 1H) 3.14 (br d, J=3.6 Hz, 1H) 2.18 (br t, J=7.4 Hz, 2H) 1.93-2.07 (m, 2H) 1.72-1.90 (m, 2H) 1.48-1.57 (m, 2H) 1.22-1.29 (m, 2H) 1.20 (s, 6H).

### 2.97 Synthesis of Compound 48

To a solution of Compound 48D (1.0 g, 2.23 mmol, 1 eq) in THF (3 mL), MeOH (3 mL) and H₂O (3 mL) was added LiOH·H₂O (141 mg, 3.35 mmol) at 20° C. The mixture was stirred at 20° C. for 1 h. The reaction mixture was concentrated under reduced pressure to remove organics. The aqueous phase was adjusted to pH=5 with 1 M HCl, the resulting solid filtered and the filter cake dried under reduced pressure to give compound 48 (850 mg, 88% yield) as a white solid; LCMS [M+H]+=434.5; ¹H NMR (400 MHz, DMSO-d₆) δ 8.08 (t, J=5.8 Hz, 1H), 7.27-7.12 (m, 2H), 7.09 (d, J=8.6 Hz, 2H), 6.67 (d, J=8.6 Hz, 2H), 3.74 (d, J=5.8 Hz, 2H), 3.70-3.56 (m, 2H), 3.54-3.29 (m, 2H), 3.23 (brt, J=10.1 Hz, 1H), 3.13 (br t, J=10.1 Hz, 1H), 2.17 (brt, J=7.4 Hz, 2H), 2.08-1.92 (m, 2H), 1.90-1.71 (m, 2H), 1.56-1.46 (m, 2H), 1.18 (s, 6H).

### C1. Synthesis of Intermediate 60

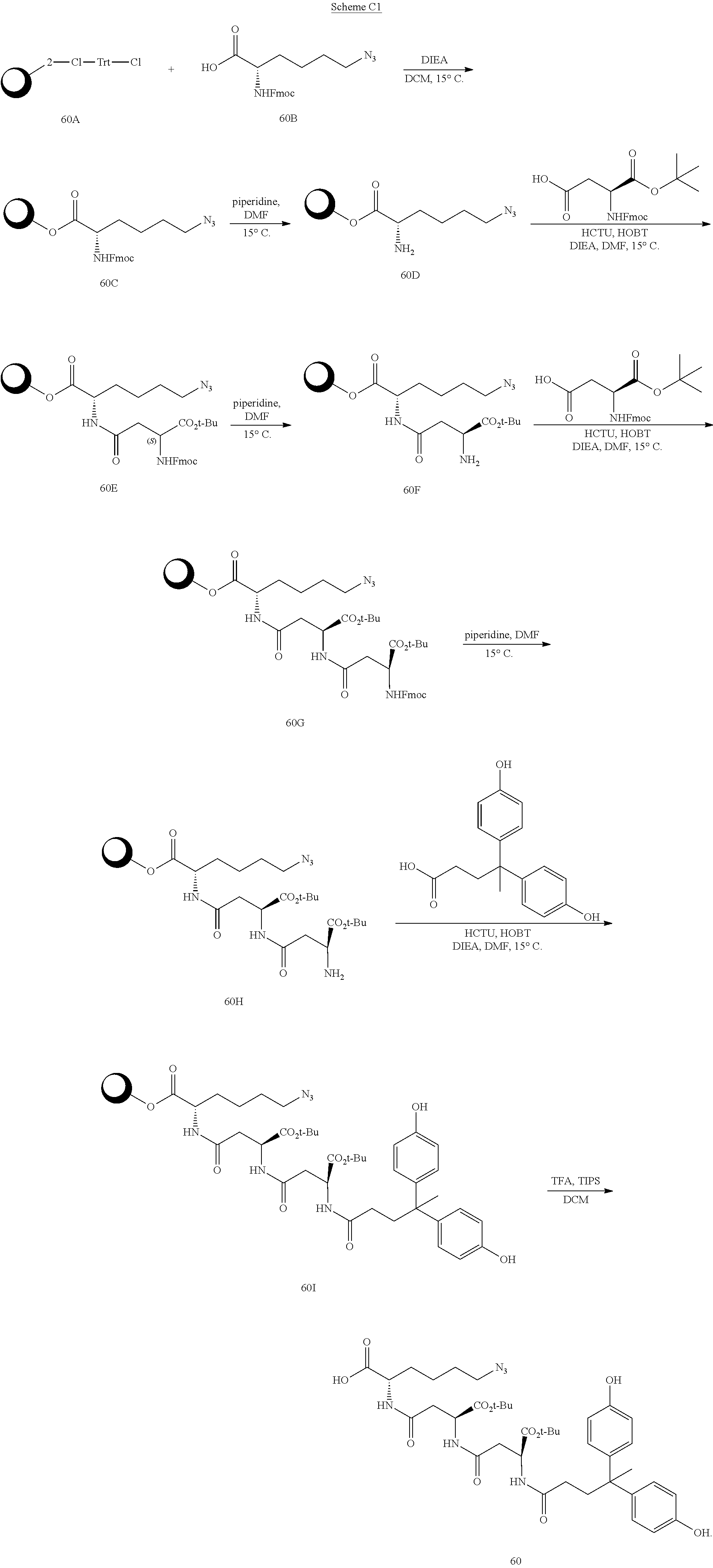

### 3.1 Preparation of Compound 60C

To a mixture of 2-Cl-Trt-Cl resin (15.5 g, 1.09 mmol/g) in DCM (100 mL) was added DIEA (17.5 g, 135 mmol, 8 eq) and compound 60B in DCM (100 mL) at 15° C., and the mixture was shaken at 15° C. for 16 h under a nitrogen atmosphere. The reaction mixture was filtered, and the filter cake washed with DCM (3×300 mL), DMF (2×300 mL), MeOH (3×300 mL) and DMF (2×300 mL), the resin dried with nitrogen and used directly in the next reaction.

### 3.2 Preparation of Compound 60D

A mixture of compound 60C in piperidine (20 mL) and DMF (80 mL) was shaken at 15° C. for 10 min under a nitrogen atmosphere. The procedure was repeated four times, the cake washed with DCM (2×300 mL) and DMF (2×300 mL), the resin dried with nitrogen and used directly in the next reaction.

### 3.3 Preparation of Compound 60E

A mixture of compound 60D (10.3 g, 25.1 mmol, 1.5 eq), HCTU (10.4 g, 25.1 mmol, 1.5 eq), HOBt (3.39 g, 25.1 mmol, 1.5 eq) and DIEA (6.49 g, 50.2 mmol, 3 eq) in DMF (100 mL) was stirred at 15° C. for 30 min. The solution was added to a solution of compound 60E in DMF (100 mL) at 15° C. and the mixture shaken at 15° C. for 2 h. The mixture was filtered, the cake washed with DMF (200 mL), DCM (3×200 mL) and DMF (2×200 mL), the resin dried with nitrogen and used directly in the next reaction.

### 3.4 Preparation of Compound 60F

A mixture of compound 60E in piperidine (20 mL) and DMF (80 mL) was shaken at 15° C. for 10 min under a nitrogen atmosphere. The procedure was repeated four times, the cake washed with DCM (2× 300 mL) and DMF (2× 300 mL), the resin dried with nitrogen and used directly in the next reaction.

### 3.5 Preparation of Compound 60G

A mixture of compound 60F (10.3 g, 24.9 mmol, 1.5 eq), HCTU (10.3 g, 24.9 mmol, 1.5 eq), HOBt (3.37 g, 24.9 mmol, 1.5 eq), DIEA (8.68 mL, 49.8 mmol, 3 eq) in DMF (100 mL) was stirred at 15° C. for 30 min. The mixture was added to a solution of(S)-3-((((9H-fluoren-9-yl)methoxy)carbonyl)amino)-4-(tert-butoxy)-4-oxobutanoic acid in DMF (100 mL) and shaken at 15° C. for 2 h. The mixture was filtered, the cake washed with DMF (100 mL), DCM (3×200 mL) and DMF (2× 100 mL), the resin dried with nitrogen and used directly in the next reaction.

### 3.6 Preparation of Compound 60H

A mixture of compound 60G in piperidine (20 mL) and DMF (80 mL) was shaken at 15° C. for 2 h under a nitrogen atmosphere. The procedure was repeated four times, the cake washed with DCM (2×30 mL) and DMF (2× 30 mL), the resin dried with nitrogen and used directly in the next reaction.

### 3.7 Preparation of Compound 601

A mixture of 4,4-bis(4-hydroxyphenyl)pentanoic acid (4.74 g, 16.6 mmol, 1 eq), HCTU (6.85 g, 16.6 mmol, 1 eq), HOBt (2.24 g, 16.6 mmol, 1 eq) and DIEA (8.65 mL, 49.7 mmol, 3 eq) in DMF (60 mL) was stirred at 15° C. for 1 min. The mixture was added to a solution of compound 60H in DMF (130 mL) and shaken at 15° C. for 0.5 h. The mixture was filtered, the cake washed with DMF (300 mL), DCM (3×300 mL), DMF (2×300 mL) and DCM (2×300 mL), the resin dried with nitrogen and used directly in the next reaction.

### 3.8 Preparation of Intermediate 60

To a solution of compound 601 in DCM (100 mL) was added TFA (1 mL) and TIPS (2.5 mL), and the reaction mixture stirred at 15° C. for 10 min. The procedure was repeated three times, THF (100 mL) added and stirred at 15° C. for 10 min. The mixture was filtered, the filtrate diluted in MeOH, washed with NaHCO₃ (3.4 g in 50 mL H₂O), the organic layer dried (Na₂SO₄), filtered and concentrated under reduced pressure. The residue was purified by column chromatography (SiO₂, Petroleum ether:EtOAc) to give intermediate 60 (14 g, purity 85%). A portion (7.0 g) of intermediate 60 was further purified by prep-HPLC (column: Phenomenex luna C18 (250*70 mm, 15 μm); mobile phase: [water (FA)-ACN]; B %: 40%-70%, 20 min) to give intermediate 60 (2.7 g) as a white solid; LCMS [M+H]: 783.4; ¹H NMR (400 MHz, DMSO-d₆) δ 12.63 (br s, 1H) 9.15 (s, 2H) 8.08-8.22 (m, 2H) 7.90 (br d, J=7.60 Hz, 1H) 6.93 (br d, J=8.40 Hz, 4H) 6.64 (br d, J=8.80 Hz, 4H) 4.41 (quin, J=6.80 Hz, 2H) 4.19 (td, J=8.00, 5.20 Hz, 1H) 3.29 (t, J=6.40 Hz, 2H) 2.52-2.60 (m, 3H) 2.39-2.47 (m, 1H) 2.12-2.23 (m, 2H) 1.80-1.91 (m, 2H) 1.63-1.76 (m, 1H) 1.48-1.62 (m, 3H) 1.46 (s, 3H) 1.35 (s, 20H).

### C2. Synthesis of Intermediate 61

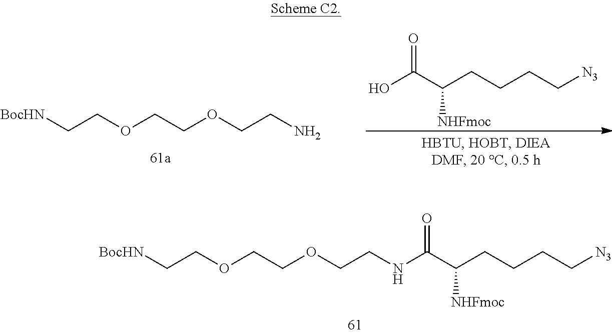

### 3.9 Preparation of Intermediate 61

To a solution of N2-(((9H-fluoren-9-yl)methoxy)carbonyl)-N6-diazo-L-lysine (1.0 g, 2.54 mmol, 1 eq) in DMF (10 mL) was added HBTU (962 mg, 2.54 mmol, 1 eq), HOBt (343 mg, 2.54 mmol, 1 eq) and DIEA (1.32 mL, 7.61 mmol, 3 eq). Compound 61A (755 mg, 3.04 mmol, 1.2 eq) was added to the mixture, the reaction stirred at 20° C. for 0.5 h, diluted with H₂O (20 mL) and extracted with EtOAc (3×50 mL). The combined organic layers were washed with brine (30 mL), dried (Na₂SO₄), filtered and concentrated under reduced pressure. The residue was purified by column chromatography (SiO₂, petroleum ether:EtOAc) to give Intermediate 61 (1.55 g, 83% yield, 84.6% purity) as a light yellow oil; LCMS [M+H]: 625.3; ¹H NMR (400 MHz, CHLOROFORM-d) δ 7.77 (d, J=7.6 Hz, 2H), 7.60 (br d, J=7.4 Hz, 2H), 7.47-7.36 (m, 2H), 7.35-7.28 (m, 2H), 6.73-6.49 (m, 1H), 5.73-5.39 (m, 1H), 5.16-4.82 (m, 1H), 4.53-4.27 (m, 2H), 4.25-4.19 (m, 1H), 3.68-3.49 (m, 9H), 3.47-3.38 (m, 1H), 3.27 (br t, J=6.6 Hz, 4H), 1.92-1.79 (m, 3H), 1.62 (dt, J=6.6, 13.6 Hz, 2H), 1.43 (br s, 11H).

Compounds in table 8.1 were prepared by procedures depicted in scheme C².

TABLE 8.1

| Intermediate | Structure | m/z |
|---|---|---|
| 61.1 | 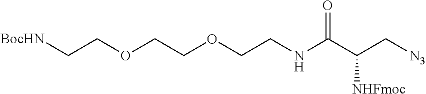 | [M + H]: 583.4 |

### C3. Synthesis of Intermediate 62

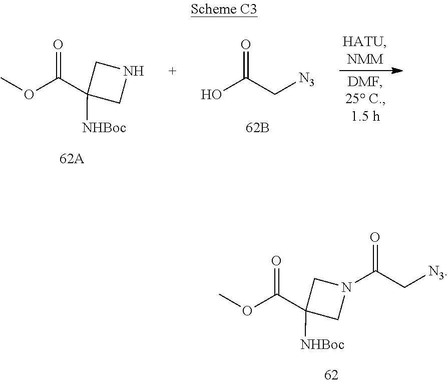 [SMILES: COC(=O)C1(NC(=O)OC(C)(C)C)CNC1 ; [N-]=[N+]=NCC(=O)O ; COC(=O)C1(NC(=O)OC(C)(C)C)CN(C(=O)CN=[N+]=[N-])C1]

### 3.10 Synthesis of Intermediate 62

To a solution of compound 62B in DMF (60 mL) was added HATU (11.9 g, 31.3 mmol, 1.2 eq) at −30° C. and the mixture was stirred at 25° C. for 0.5 h. A solution of compound 62A (6.0 g, 26.1 mmol, 1 eq) in DMF (6 mL) and NMM (8.59 mL, 78.2 mmol, 3 eq) was added and the mixture stirred at 25° C. for 1 h. The mixture was extracted with EtOAc (2×200 mL), the combined organic layer washed with brine (150 mL), dried (Na₂SO₄), filtered and concentrated under reduced pressure. The residue was purified by column chromatography (SiO₂, Petroleum ether:EtOAc) to give compound 62 (5.0 g, 61% yield) as a white solid.

### C4. Synthesis of Intermediate 63

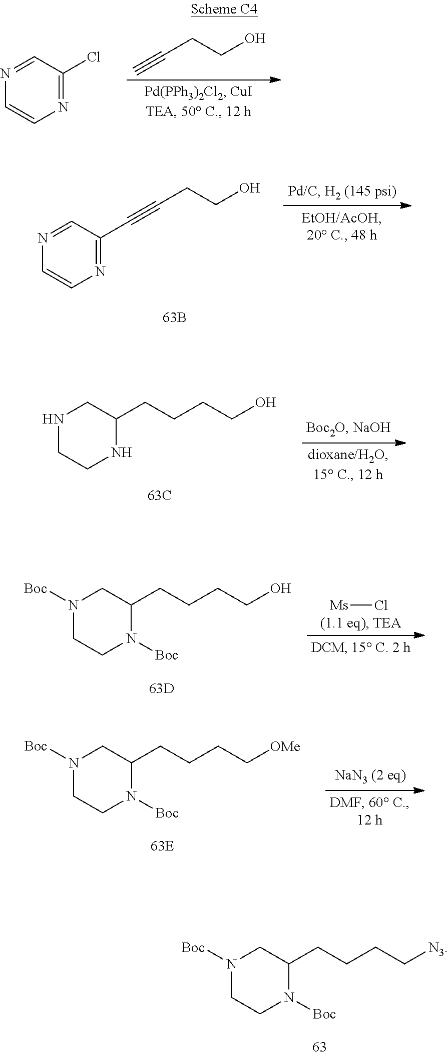

### 3.11 Synthesis of Compound 63B

To a solution of 2-chloropyrazine (7.81 mL, 87.3 mmol, 1 eq) and but-3-yn-1-ol (8.59 mL, 114 mmol, 1.3 eq) in TEA (50 mL) was added CuI (299 mg, 1.57 mmol, 0.018 eq) and Pd(PPh₃)₂Cl₂ (1.10 g, 1.57 mmol, 0.018 eq) at 15° C., and the mixture stirred in a sealed tube at 50° C. for 12 h. The reaction mixture was concentrated under reduced pressure and purified by column chromatography (SiO₂, petroleum ether:EtOAc) to give compound 63B (21.8 g, 84% yield) as a yellow solid; LCMS [M+H]: 149.2; ¹H NMR (400 MHz, CHLOROFORM-d) δ 8.64 (s, 1H), 8.53-8.49 (m, 1H), 8.46 (d, J=2.4 Hz, 1H), 3.89 (t, J=6.3 Hz, 2H), 2.77 (t, J=6.3 Hz, 2H), 2.49 (br s, 1H).

### 3.12 Synthesis of Compound 63C

To a solution of compound 63B (20 g, 135 mmol, 1 eq) in EtOH (250 mL) and AcOH (10 mL) was added Pd/C (10 g, 10% purity) under a nitrogen atmosphere. The mixture was stirred at 20° C. under a hydrogen atmosphere (145 psi) for 48 h. The mixture was filtered and concentrated to give crude compound 63C (40 g) as a yellow solid; LCMS [M+H]: 159.5; ¹H NMR (400 MHz, DMSO-d₆) δ 3.37 (t, J=6.3 Hz, 1H), 2.97-2.81 (m, 2H), 2.78-2.57 (m, 2H), 2.32 (t, J=11.4 Hz, 1H), 1.84 (s, 7H), 1.44-1.21 (m, 4H).

### 3.13 Synthesis of Compound 63D

To a solution of compound 63C (30 g, 190 mmol, 1 eq) in dioxane (240 mL) and H₂O (90 mL) was added NaOH (22.8 g, 569 mmol, 3 eq) and Boc2O (109 mL, 474 mmol, 2.5 eq) at 15° C., and the mixture stirred at 15° C. for 12 h. The reaction mixture was quenched by addition of a saturated NH₄Cl solution (100 mL) at 20° C. and extracted with DCM (2×80 mL). The combined organic layers were dried (Na₂SO₄), filtered and concentrated under reduced pressure. The residue was purified by column chromatography (SiO₂, petroleum ether:EtOAc) to give compound 63D (30 g, 44% yield) as yellow oil; LCMS [M−155]: 203.2; ¹H NMR (400 MHz, DMSO-d₆) δ 4.35 (t, J=4.6 Hz, 1H), 4.03-3.84 (m, 2H), 3.81 (d, J=13.4 Hz, 1H), 3.71 (d, J=11.3 Hz, 1H), 3.37 (q, 2H), 3.31 (s, 1H), 2.99-2.63 (m, 3H), 1.39 (s, 18H), 1.38-1.32 (m, 2H), 1.32-1.17 (m, 2H).

### 3.14 Synthesis of Compound 63E

To a solution of compound 63D (5.0 g, 14.0 mmol, 1 eq) in DCM (50 mL) was added TEA (2.91 mL, 20.9 mmol, 1.5 eq) and MsCl (1.19 mL, 15.3 mmol, 1.1 eq) at 0° C., and the mixture stirred at 15° C. for 2 h. The reaction mixture was quenched with aq. NH₄Cl (100 mL) and extracted with DCM (2× 50 mL). The combined organic layers were dried (Na₂SO₄), filtered and concentrated under reduced pressure to give crude compound 63E (6.0 g) as a yellow oil; ¹H NMR (400 MHz, DMSO-d₆) δ 4.18 (t, J=6.3 Hz, 2H), 4.06-3.66 (m, 4H), 3.32 (s, 1H), 3.14 (s, 3H), 3.05 (q, 6H), 1.68 (q, 2H), 1.40 (s, 18H).

### 3.15 Synthesis of Compound 63

To a solution of compound 63E (36 g, 82.5 mmol, 1 eq) in DMF (150 mL) was added NaN₃ (10.7 g, 165 mmol, 2 eq) at 15° C., and the mixture stirred at 60° C. for 12 h. The mixture was diluted with water (300 mL) and extracted with EtOAc (300 mL). The organic layer was washed with water (2×100 mL), dried (Na₂SO₄) and concentrated under reduced pressure. The residue was purified by column chromatography (SiO₂, petroleum ether:EtOAc) to give compound 63 (22 g, 70% yield) as a colorless oil; ¹H NMR (400 MHz, DMSO-d₆) δ 4.04-3.84 (m, 2H), 3.80 (d, J=13.4 Hz, 2H), 3.73 (d, J=11.2 Hz, 1H), 3.03-2.60 (m, 4H), 1.55 (q, 3H), 1.48-1.45 (m, 1H), 1.40 (s, 18H), 1.34-1.27 (m, 1H), 1.27-1.19 (m, 1H).

### C5. Synthesis of Intermediate 64

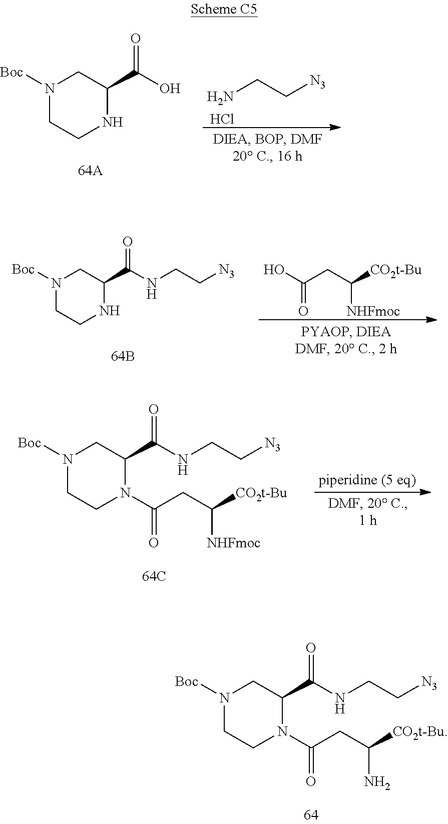

### 3.16 Preparation of Compound 64B

To a solution of 2-azidoethan-1-amine hydrochloride (13.3 g, 109 mmol, 1.0 eq) in DMF (250 mL) was added DIEA (47.3 mL, 271 mmol, 2.5 eq), compound 64A (25 g, 109 mmol, 1 eq) and BOP (52.8 g, 119 mmol, 1.1 eq), and the mixture stirred at 20° C. for 16 h. The reaction mixture was quenched by addition of H₂O (10 mL) at 20° C., diluted with H₂O (250 mL) and extracted with EtOAc (2×300 mL). The combined organic layers were washed with a saturated NH₄Cl solution (150 mL), NaHCO₃ solution (150 mL) and brine (250 mL), dried (Na₂SO₄), filtered and concentrated under reduced pressure. The residue was purified by column chromatography (SiO₂, DCM:MeOH) to give compound 64B (27.4 g, 63% yield, 75% purity) as an orange oil; ¹H NMR (400 MHz, CHLOROFORM-d) δ 7.24 (br s, 1H), 4.11-3.99 (m, 1H), 3.51-3.39 (m, 5H), 3.21-3.12 (m, 3H), 3.04 (br d, J=12.8 Hz, 2H), 2.92-2.80 (m, 1H), 1.46 (s, 9H).

### 3.17 Synthesis of Intermediate 64C

To a solution of compound 64B (6.00 g, 12.1 mmol, 60% purity, 1 eq), (S)-3-((((9H-fluoren-9-yl)methoxy)carbonyl)amino)-4-(tert-butoxy)-4-oxobutanoic acid (4.96 g, 12.07 mmol, 1 eq) in DMF (60 mL) was added DIEA (4.20 mL, 24.1 mmol, 2 eq) and PyAOP (6.92 g, 13.3 mmol, 1.1 eq). The mixture was stirred at 20° C. for 2 h, quenched by the addition of H₂O (50 mL) at 20° C. and extracted with EtOAc (2×100 mL). The combined organic layers were washed with brine (20 mL), dried (Na₂SO₄), filtered and concentrated under reduced pressure. The residue was purified by column chromatography (SiO₂, petroleum ether:EtOAc) to give compound 64C (8.0 g, 96% yield) as a white solid; LCMS [M+H]: 692; ¹H NMR (400 MHz, DMSO-d₆) δ 8.33 (br s, 1H), 8.17-8.11 (m, 1H), 7.89 (br d, J=7.4 Hz, 2H), 7.70 (br d, J=7.4 Hz, 2H), 7.46-7.38 (m, 3H), 7.36-7.27 (m, 2H), 4.75 (brs, 1H), 4.41-4.14 (m, 5H), 3.73 (br d, J=11.8 Hz, 2H), 3.40-3.30 (m, 4H), 3.27-3.13 (m, 2H), 1.45-1.30 (m, 18H).

### 3.18 Synthesis of Intermediate 64

To a solution of compound 64C (5.0 g, 7.23 mmol, 1 eq) in DMF (60 mL) was added piperidine (3.57 mL, 36.1 mmol, 5 eq), the mixture stirred at 20° C. for 1 h and concentrated under reduced pressure. The residue was purified by column chromatography (SiO₂, DCM:MeOH) to give crude Intermediate 64 (3.6 g) as a white solid; LCMS [M+H]: 470.3; ¹H NMR (400 MHz, DMSO-d₆) δ 8.42-8.19 (m, 1H), 4.28 (br d, J=13.8 Hz, 1H), 3.85-3.69 (m, 3H), 3.56-3.41 (m, 2H), 3.30-3.07 (m, 5H), 3.04-2.98 (m, 1H), 2.86-2.76 (m, 2H), 1.49-1.29 (m, 18H).

Compounds in table 8.2 were prepared by procedures depicted in scheme C⁵.

TABLE 8.2

| Inter- | | |
|---|---|---|
| mediate | Structure | m/z |
| 64.1 | 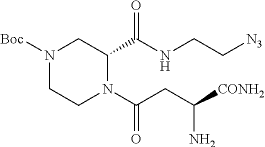 [SMILES: CC(C)(C)OC(=O)N1CCN(C(=O)C[C@H](N)C(N)=O)[C@@H](C(=O)NCCN=[N+]=[N-])C1] | [M + H]: 413.3 |
| 64.2 | 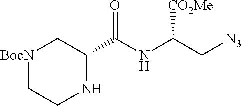 [SMILES: COC(=O)[C@H](CN=[N+]=[N-])NC(=O)[C@H]1CN(C(=O)OC(C)(C)C)CCN1] | [M + H]: 357.3 |
| 64.3 | 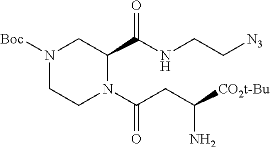 [SMILES: CC(C)(C)OC(=O)[C@@H](N)CC(=O)N1CCN(C(=O)OC(C)(C)C)C[C@H]1C(=O)NCCN=[N+]=[N-]] | [M + H]: 470.3 |

### C6. Synthesis of Intermediate 65

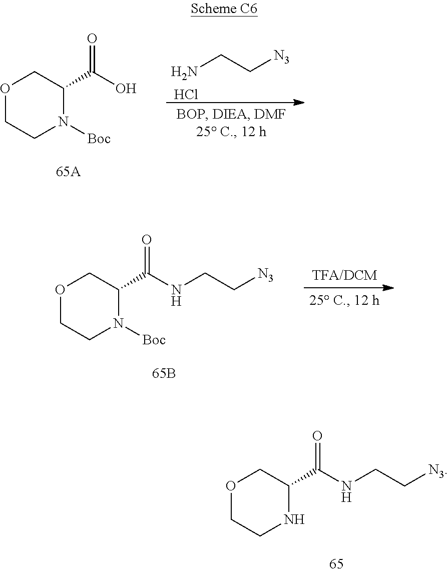 [SMILES: CC(C)(C)OC(=O)N1CCOC[C@@H]1C(=O)O ; Cl ; [N-]=[N+]=NCCN ; CC(C)(C)OC(=O)N1CCOC[C@@H]1C(=O)NCCN=[N+]=[N-] ; [N-]=[N+]=NCCNC(=O)[C@H]1COCCN1]

### 3.19 Preparation of Compound 65B

To a solution of compound 65A (10 g, 43.2 mmol, 1 eq), 2-azidoethan-1-amine hydrochloride (5.30 g, 43.2 mmol, 1 eq) in DMF (50 mL) was added BOP (21.0 g, 47.6 mmol, 1.1 eq) and DIEA (30.1 mL, 173 mmol, 4 eq). The mixture was stirred at 25° C. for 12 h. The reaction mixture was concentrated under reduced pressure and the residue purified by column chromatography (SiO₂, petroleum ether:EtOAc) to give compound 65B (11.4 g, 88% yield) as a yellow oil; LCMS [M−100]: 200; ¹H NMR (400 MHz, DMSO-d₆) δ 8.15 (br s, 1H), 4.31-4.09 (m, 2H), 3.76 (br s, 1H), 3.61-3.50 (m, 2H), 3.36 (br s, 3H), 3.24 (br s, 2H), 1.36 (br s, 9H).

### 3.20 Preparation of Intermediate 65

To a solution of compound 65B (11.4 g, 38.2 mmol, 1 eq) in DCM (90 mL) was added TFA (30 mL) and the mixture stirred at 25° C. for 12 h. The reaction mixture was concentrated under reduced pressure to give crude Intermediate 65 (7.61 g) as an oil; LCMS [M+H]: 200.2; ¹H NMR (400 MHz, CHLOROFORM-d) δ 8.51 (br d, J=4.5 Hz, 1H), 4.52 (br dd, J=3.7, 10.5 Hz, 1H), 4.24-4.17 (m, 1H), 4.02 (br d, J=11.7 Hz, 1H), 3.87 (br t, J=11.7 Hz, 1H), 3.75 (br t, J=11.6 Hz, 1H), 3.70-3.60 (m, 1H), 3.48 (br dd, J=4.2, 10.4 Hz, 3H), 3.40-3.33 (m, 3H).

### C7. Synthesis of Intermediate 66

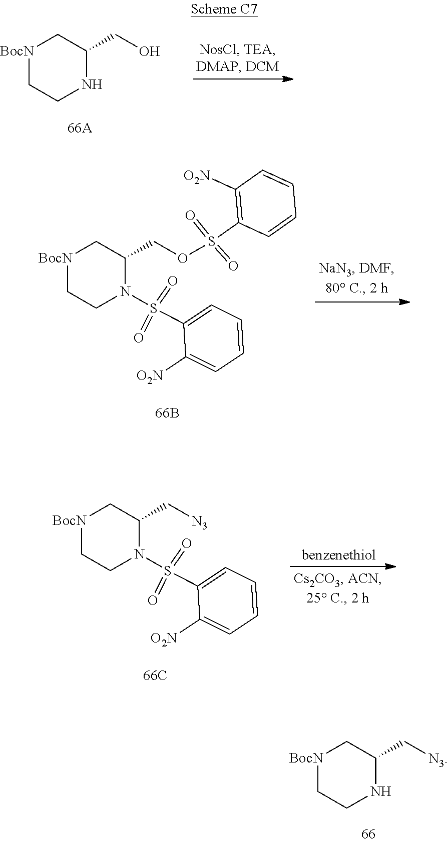 [SMILES: CC(C)(C)OC(=O)N1CCN[C@@H](CO)C1 ; CC(C)(C)OC(=O)N1CCN(S(=O)(=O)c2ccccc2[N+](=O)[O-])[C@@H](COS(=O)(=O)c2ccccc2[N+](=O)[O-])C1 ; CC(C)(C)OC(=O)N1CCN(S(=O)(=O)c2ccccc2[N+](=O)[O-])[C@@H](CN=[N+]=[N-])C1 ; CC(C)(C)OC(=O)N1CCN[C@@H](CN=[N+]=[N-])C1]

### 3.21 Preparation of Compound 66B

To a solution of compound 66A (25 g, 116 mmol, 1 eq) in DCM (500 mL) was added NosCI (79.4 g, 358 mmol, 3.1 eq), DMAP (141 mg, 1.16 mmol, 0.01 eq) and TEA (64.4 mL, 462 mmol, 4 eq) at 25° C. The mixture was stirred at 25° C. for 12 h, poured into water (500 mL) and the aqueous phase extracted with DCM (3× 500 mL). The combined organic phase was washed with brine (2×500 mL), dried (Na₂SO₄), filtered and concentrated under reduced pressure. The residue was purified by prep-HPLC (column: Welch Xtimate C18 250*100 mm #10 μm; mobile phase: [water (NH₄HCO₃)-ACN]; B %: 45%-75%, 20 min) to give compound 66B (40 g, 59% yield) as a yellow solid.

### 3.22 Preparation of Compound 66C

To a solution of compound 66B (10 g, 17.1 mmol, 1 eq) in DMF (100 mL) was added NaN₃ (2.22 g, 34.1 mmol, 2 eq) and the mixture stirred at 80° C. for 2 h. The mixture was diluted with water (500 mL) and extracted with EtOAc (300 mL). The organic layer was washed with brine (2×200 mL), dried (Na₂SO₄), filtered and concentrated under reduced pressure. The residue was purified by column chromatography (SiO₂, petroleum ether:EtOAc) to give compound 66C (8.4 g, 58% yield) as a light yellow solid; ¹H NMR (400 MHz, CDCl₃) δ 8.19-8.04 (m, 1H), 7.87-7.64 (m, 3H), 4.22-4.11 (m, 1H), 3.99 (s, 1H), 3.79-3.58 (m, 1H), 3.54-3.37 (m, 2H), 3.27-3.15 (m, 1H), 3.03 (d, J=11.8 Hz, 1H), 2.93-2.66 (m, 1H), 1.52-1.42 (m, 9H).

### 3.23 Preparation of Intermediate 66

To a mixture of compound 66C (8.0 g, 18.8 mmol, 1 eq) in ACN (80 mL) was added benzenethiol (3.83 mL, 37.5 mmol, 2 eq) and Cs₂CO₃ (12.2 g, 37.5 mmol, 2 eq) at 25° C. under a nitrogen atmosphere. The mixture was stirred at 25° C. for 2 h. The reaction mixture was quenched by addition of a 2 M NaOH solution (200 mL) at 25° C. and extracted with EtOAc (2×200 mL). The combined organic layers were washed with brine (200 mL), dried (Na₂SO₄) and concentrated under reduced pressure. The residue was purified by column chromatography (SiO₂, petroleum ether:EtOAc) to give compound 66 (8.2 g, 56% yield) as a light yellow solid; ¹H NMR (400 MHz, CDCl₃) δ 3.89 (d, J=7.8 Hz, 2H), 3.42 (dd, J=4.4, 12.2 Hz, 1H), 3.26 (dd, J=7.7, 12.1 Hz, 1H), 2.98 (d, J=11.5 Hz, 1H), 2.93-2.84 (m, 1H), 2.83-2.69 (m, 2H), 2.61 (s, 1H), 1.46 (s, 9H).

### C8. Synthesis of Intermediate 9

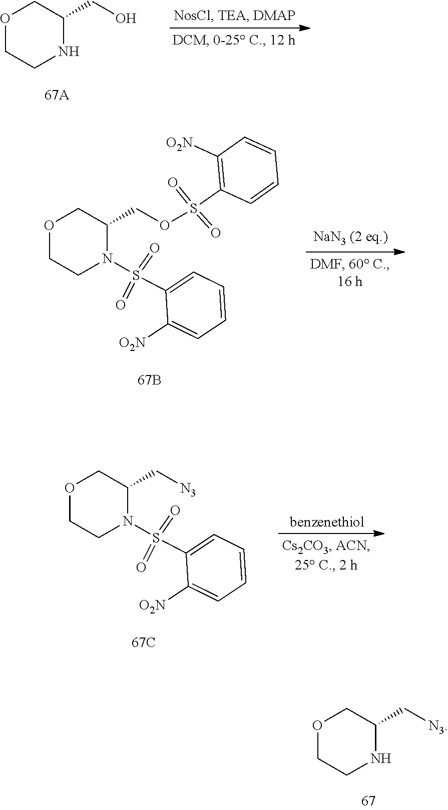 [SMILES: OC[C@H]1COCCN1 ; O=[N+]([O-])c1ccccc1S(=O)(=O)OC[C@H]1COCCN1S(=O)(=O)c1ccccc1[N+](=O)[O-] ; [N-]=[N+]=NC[C@H]1COCCN1S(=O)(=O)c1ccccc1[N+](=O)[O-] ; [N-]=[N+]=NC[C@H]1COCCN1]

### 3.24 Preparation of Compound 67B

To a solution of compound 67A (6.0 g, 39.1 mmol, 1 eq) in DCM (60 mL) was added TEA (21.8 mL, 156 mmol, 4 eq) and DMAP (48 mg, 391 μmol, 0.01 eq). 2-Nitrobenzenesulfonyl chloride (26.8 g, 121 mmol) was added at 0° C., the mixture stirred at 25° C. for 12 h, extracted with DCM (2×10 mL), the combined organic phase washed with brine (10 mL), dried (Na₂SO₄) and concentrated under reduced pressure. The crude product was purified by silica gel column chromatography (petroleum ether:EtOAc) to give compound 67B (11 g, 56% yield) as a yellow oil; ¹H NMR (400 MHz, DMSO-d₆) δ 8.09-8.17 (m, 3H), 8.04 (td, J=7.75, 1.38 Hz, 1H), 7.92-7.99 (m, 2H), 7.79-7.92 (m, 2H), 4.99 (br s, 1H), 4.51 (dd, J=10.19, 7.07 Hz, 1H), 4.37 (dd, J=10.19, 7.69 Hz, 1H), 4.12 (td, J=7.19, 2.63 Hz, 1H), 3.85 (d, J=12.13 Hz, 1H), 3.77 (dd, J=11.51, 2.63 Hz, 1H), 3.50-3.56 (m, 1H), 3.38-3.47 (m, 1H), 3.16-3.25 (m, 1H).

### 3.25 Preparation of Compound 67C

To a solution of compound 67B (4.0 g, 8.21 mmol, 1 eq) in DMF (40 mL) was added NaN₃ (1.07 g, 16.4 mmol, 2 eq) at 25° C. The mixture was stirred at 60° C. for 16 h, quenched by addition of a saturated K₂CO₃ solution at 25° C. until the reaction mixture pH=9, and extracted with EtOAc (2×20 mL). The combined organic phase was washed with brine (20 mL), dried (Na₂SO₄) and concentrated under reduced pressure. (The aqueous phase was quenched with sodium hypochlorite aqueous solution (160 ml)). The residue was purified by silica gel chromatography (petroleum ether:EtOAc) to give compound 67C (3.8 g, 69% yield) as a yellow oil; ¹H NMR (400 MHz, DMSO-d₆) δ 8.15 (dd, J=7.72, 1.57 Hz, 1H), 7.98-8.03 (m, 1H), 7.83-7.95 (m, 2H), 3.91-3.98 (m, 1H), 3.67-3.82 (m, 3H), 3.52-3.64 (m, 2H), 3.37-3.48 (m, 2H), 3.19 (td, J=11.86, 3.14 Hz, 1H).

### 3.26 Preparation of Intermediate 67

To a solution of compound 67C (1.0 g, 3.06 mmol, 1 eq) in ACN (14 mL) was added Cs₂CO₃ (1.99 g, 6.11 mmol, 2 eq) and benzenethiol (623 μL, 6.11 mmol, 2 eq). The mixture was stirred at 25° C. for 2 h, quenched by addition of a 2 M NaOH solution at 25° C. (15 mL), and extracted with EtOAc (2×20 mL). The combined organic layers were washed with brine (20 mL), dried (Na₂SO₄) and concentrated under reduced pressure. (The aqueous phase was quenched with sodium hypochlorite aqueous solution). The residue was purified by column chromatography (SiO₂, petroleum ether:EtOAc) to give Intermediate 67 (500 mg, 69% yield, 60% purity) as a yellow oil; ¹H NMR (400 MHz, DMSO-d₆) δ 3.59-3.69 (m, 2H), 3.23-3.36 (m, 2H), 3.16-3.23 (m, 1H), 3.10 (dd, J=10.8, 9.3 Hz, 1H), 2.76-2.83 (m, 1H), 2.65-2.75 (m, 2H).

### C9. Synthesis of Intermediate 68

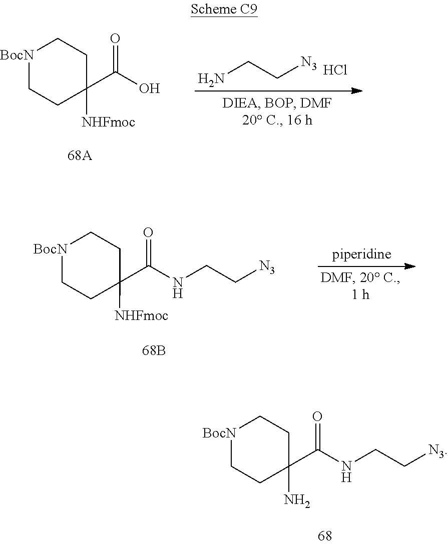

### 3.27 Preparation of Compound 68B

To a mixture of 2-azidoethan-1-amine HCl (1.31 g, 10.7 mmol, 1 eq) in DMF (40 mL) was added DIEA (2.1 mL, 11.8 mmol, 1.1 eq) and the mixture stirred at 20° C. for 10 minutes. To the reaction mixture was added compound 68A (5.0 g, 10.72 mmol, 1 eq) and BOP (4.74 g, 10.72 mmol, 1 eq) at 20° C. The mixture was stirred at 20° C. for 16 h, poured into water (50 mL), the aqueous phase extracted with EtOAc (3×200 mL), the combined organics washed with brine (2×100 mL), dried (Na₂SO₄), filtered and concentrated under reduced pressure. The residue was purified by column chromatography (SiO₂, petroleum ether:EtOAc) to give compound 68B (11.8 g, 69% yield) as a white solid; ¹H NMR (400 MHz, DMSO-d₆) δ 7.95-7.86 (m, 2H), 7.82 (br s, 1H), 7.74 (br d, J=7.2 Hz, 2H), 7.50 (brs, 1H), 7.47-7.28 (m, 4H), 4.34-4.14 (m, 3H), 3.64 (br d, J=12.0 Hz, 2H), 3.32-3.15 (m, 4H), 3.01 (br s, 2H), 1.89 (br s, 2H), 1.77 (br t, J=10.2 Hz, 2H), 1.40 (s, 9H).

### 3.28 Preparation of Intermediate 68

To a mixture of compound 68B (5.4 g, 10.1 mmol, 1 eq) in DMF (100 mL) was added piperidine (4.99 mL, 50.5 mmol, 5 eq) in one portion at 20° C. The mixture was stirred at 20° C. for 1 h and concentrated under reduced pressure. The residue was purified by column chromatography (SiO₂, petroleum ether:EtOAc) to give Intermediate 68 (4.5 g, 71% yield) as a light-yellow solid; ¹H NMR (400 MHz, CHLOROFORM-d) δ 8.04 (br s, 1H), 4.08-3.90 (m, 2H), 3.49-3.40 (m, 4H), 3.06 (br t, J=12.2 Hz, 2H), 2.16 (ddd, J=4.6, 11.8, 13.8 Hz, 2H), 1.47 (s, 9H), 1.42-1.32 (m, 2H).

### C10. Synthesis of Intermediate 69

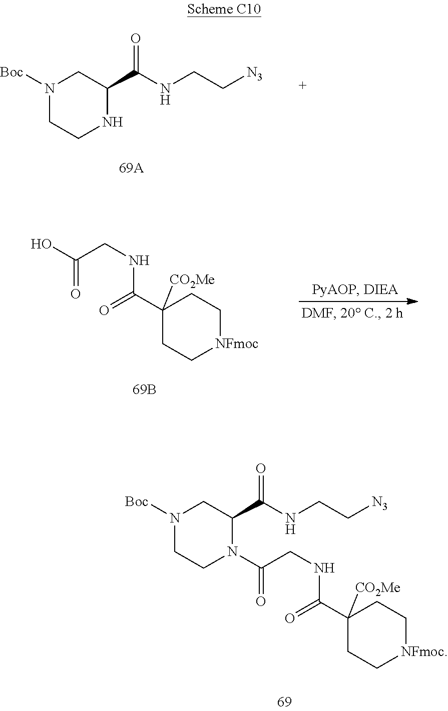

### 3.29 Synthesis of Intermediate 69

To a mixture of compound 69A (2.13 g, 4.29 mmol, 1 eq) and compound 69B (2.0 g, 4.29 mmol, 1 eq) in DMF (15 mL) was added PyAOP (2.46 g, 4.72 mmol, 1.1 eq) and DIEA (1.49 mL, 8.57 mmol, 2 eq) at 20° C. under a nitrogen atmosphere. The mixture was stirred at 20° C. for 2 h and the reaction mixture concentrated under reduced pressure. The crude product was purified by column chromatography (SiO₂, petroleum ether:EtOAc) to give crude compound 69 (7.6 g) as a white solid; LCMS [M−100]: 647.4; ¹H NMR (400 MHz, DMSO-d₆) δ 8.24-8.11 (m, 1H), 7.98-7.78 (m, 2H), 7.68-7.55 (m, 1H), 7.48-7.26 (m, 2H), 4.71 (br d, J=1.4 Hz, 1H), 4.46-4.20 (m, 2H), 4.18-4.07 (m, 1H), 3.93-3.81 (m, 1H), 3.69-3.58 (m, 3H), 3.22 (br s, 13H), 2.07-1.69 (m, 4H), 1.47-1.12 (m, 15H).

### C11. Synthesis of Intermediate 70

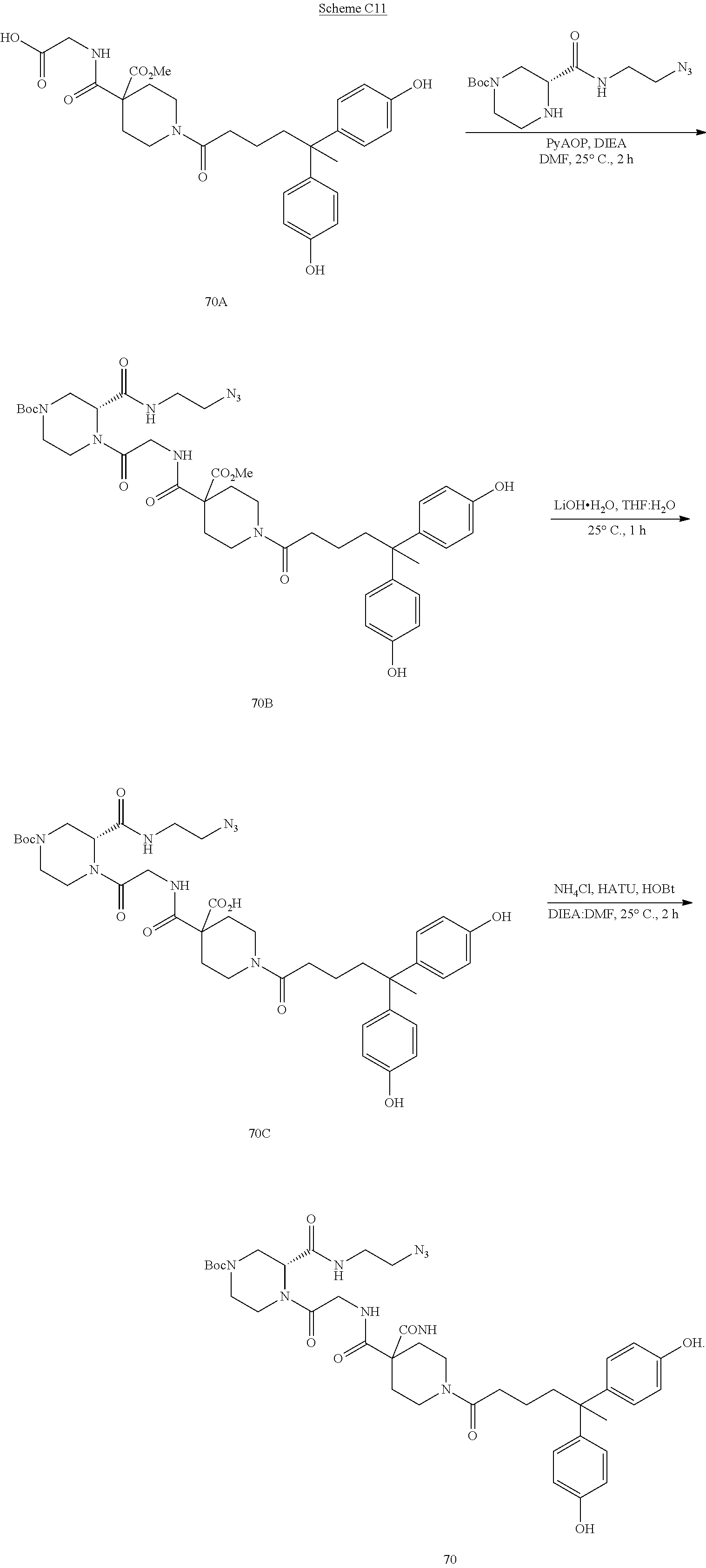

### 3.30 Synthesis of Compound 70B

To a solution of tert-butyl (R)-3-((2-azidoethyl) carbamoyl) piperazine-1-carboxylate (933 mg, 3.13 mmol, 1 eq) and compound 70A (1.65 g, 3.13 mmol, 1 eq) in DMF (18 mL) was added DIEA (1.36 mL, 7.82 mmol, 2.5 eq) and PyAOP (1.79 g, 3.44 mmol, 1.1 eq). The mixture was stirred at 25° C. for 2 h, partitioned between EtOAc (50 mL) and H₂O (10 mL), the organic phase washed with brine (2× 5 mL), dried (Na₂SO₄), filtered and concentrated under reduced pressure. The residue was purified by column chromatography (SiO₂, DCM:MeOH) to give crude compound 70B (1.62 g) as a white solid; LCMS [M+H]: 807.4; ¹H NMR (400 MHz, DMSO-d₆) δ 9.12 (s, 2H), 8.19 (br t, J=5.4 Hz, 1H), 7.93-7.86 (m, 1H), 6.93 (d, J=8.6 Hz, 4H), 6.62 (d, J=8.6 Hz, 4H), 4.71 (brs, 1H), 4.33-4.20 (m, 1H), 4.14 (br d, J=5.0 Hz, 1H), 4.07-3.98 (m, 1H), 3.94-3.81 (m, 1H), 3.74-3.61 (m, 4H), 3.58-3.42 (m, 3H), 3.39-3.33 (m, 2H), 3.30-3.20 (m, 3H), 3.17 (d, J=5.4 Hz, 2H), 3.14 (brs, 1H), 3.01 (br dd, J=2.6, 6.6 Hz, 1H), 2.30-2.21 (m, 2H), 1.97-1.89 (m, 4H), 1.47 (s, 3H), 1.37 (s, 9H), 1.28-1.17 (m, 3H).

### 3.31 Synthesis of Compound 70C

To a solution of compound 70B (1.4 g, 1.74 mmol, 1 eq) in THF (8 mL) and H₂O (8 mL) was added LiOH·H₂O (218 mg, 5.21 mmol, 3 eq). The mixture was stirred at 25° C. for 1 h, the reaction mixture concentrated under reduced pressure and the aqueous phase adjusted to pH=5-6 with aq. HCl. The reaction mixture was concentrated under reduced pressure to give compound 70C (1.36 g, 99% yield) as a white solid; LCMS [M+H]: 793.4; ¹H NMR (400 MHz, DMSO-d₆) δ 9.37-8.91 (m, 1H), 8.18 (br t, J=5.4 Hz, 1H), 7.91 (br s, 1H), 6.93 (d, J=8.6 Hz, 4H), 6.63 (d, J=8.6 Hz, 4H), 4.72 (br s, 1H), 4.31 (br d, J=12.4 Hz, 1H), 4.16-3.86 (m, 3H), 3.80-3.53 (m, 5H), 3.51-3.43 (m, 2H), 3.30-3.07 (m, 7H), 2.24 (br t, J=7.2 Hz, 2H), 2.05-1.74 (m, 7H), 1.47 (s, 4H), 1.37 (s, 9H), 1.24 (br d, J=5.4 Hz, 2H).

### 3.32 Synthesis of Intermediate 70

To a solution of compound 70C (1.36 g, 1.72 mmol, 1 eq) in DMF (14 mL) was added NH₄Cl (110 mg, 2.06 mmol, 1.2 eq), DIEA (598 μL, 3.43 mmol, 2 eq), HATU (652 mg, 1.72 mmol, 1 eq) and HOBt (278 mg, 2.06 mmol, 1.2 eq). The reaction mixture was stirred at 25° C. for 2 h and concentrated under reduced pressure. The residue was purified by reversed-phase HPLC to give intermediate 70 (900 mg, 66% yield) as a white solid; LCMS [M+H]: 792.7; ¹H NMR (400 MHz, DMSO-d₆) δ 9.31-8.92 (m, 1H), 8.21 (br s, 1H), 7.95 (brs, 1H), 7.45 (br s, 1H), 7.24 (brs, 1H), 6.93 (d, J=8.6 Hz, 4H), 6.63 (d, J=8.6 Hz, 4H), 4.72 (br s, 1H), 4.39-4.09 (m, 2H), 3.91 (br dd, J=5.0, 16.8 Hz, 1H), 3.80-3.61 (m, 4H), 3.35 (br d, J=5.4 Hz, 3H), 3.29-3.07 (m, 6H), 2.24 (brs, 2H), 2.09-1.76 (m, 7H), 1.47 (s, 3H), 1.38 (s, 9H), 1.22 (br s, 2H).

### C12. Synthesis of Intermediate 71

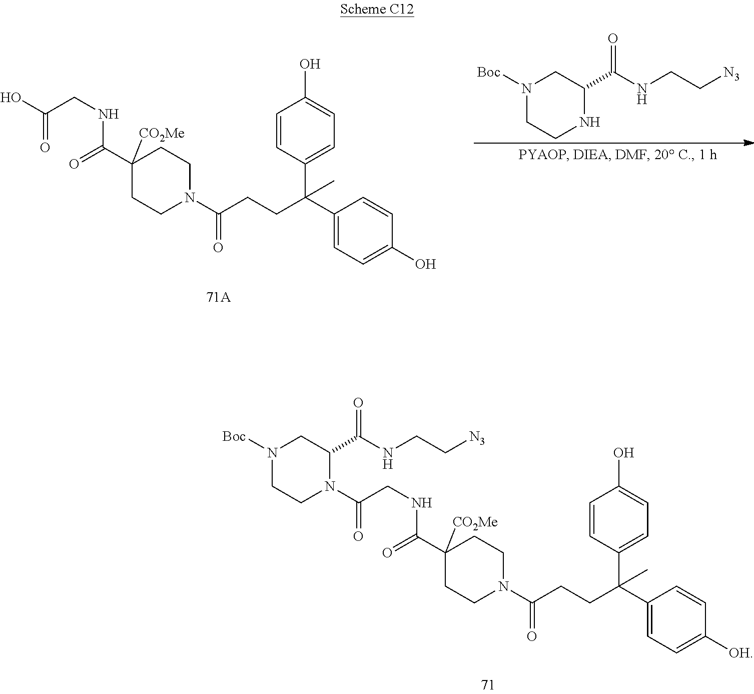 [SMILES: COC(=O)C1(C(=O)NCC(=O)O)CCN(C(=O)CCC(C)(c2ccc(O)cc2)c2ccc(O)cc2)CC1 ; CC(C)(C)OC(=O)N1CCN[C@@H](C(=O)NCCN=[N+]=[N-])C1 ; COC(=O)C1(C(=O)NCC(=O)N2CCN(C(=O)OC(C)(C)C)C[C@@H]2C(=O)NCCN=[N+]=[N-])CCN(C(=O)CCC(C)(c2ccc(O)cc2)c2ccc(O)cc2)CC1]

### 3.33 Synthesis of Intermediate 71

To a mixture of tert-butyl (R)-3-((2-azidoethyl)carbamoyl)piperazine-1-carboxylate (450 mg, 1.51 mmol, 1 eq) and compound 71A (773 mg, 1.51 mmol, 1 eq) in DMF (5 mL) was added DIEA (657 μL, 3.77 mmol, 2.5 eq) and PyAOP (865 mg, 1.66 mmol, 1.1 eq) at 20° C. under a nitrogen atmosphere. The mixture was stirred at 20° C. for 1 h, poured into water (80 mL) and extracted with EtOAc (3×120 mL). The combined organic phase was washed with brine (2×20 mL), dried (Na₂SO₄), and concentrated under reduced pressure. The crude product was purified by column chromatography (DCM:MeOH) to give intermediate 71 (850 mg, 71% yield) as a white solid; ¹H NMR (400 MHz, METHANOL-d₄) δ 7.02 (br d, J=8.4 Hz, 5H), 6.69 (br d, J=7.6 Hz, 4H), 4.10 (q, J=7.0 Hz, 4H), 3.94-3.84 (m, 1H), 3.83-3.67 (m, 5H), 3.65-3.53 (m, 1H), 3.44-3.36 (m, 4H), 3.33-3.30 (m, 8H), 3.28-3.10 (m, 3H), 2.37-2.24 (m, 2H), 2.15-2.05 (m, 4H), 1.46 (s, 9H).

### C13. Synthesis of Intermediate 72

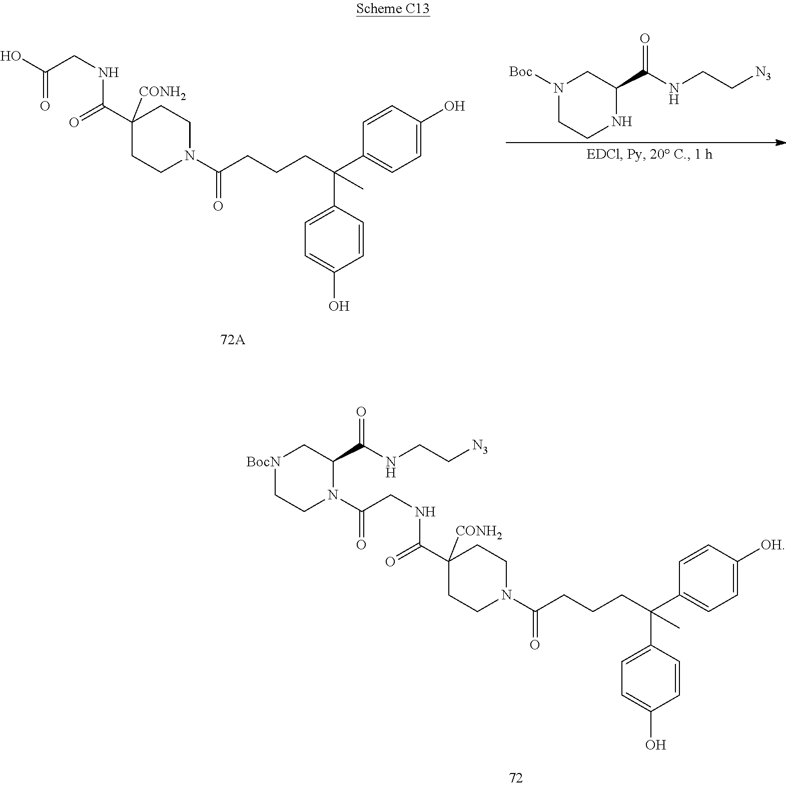 [SMILES: CC(CCCC(=O)N1CCC(C(N)=O)(C(=O)NCC(=O)O)CC1)(c1ccc(O)cc1)c1ccc(O)cc1 ; CC(C)(C)OC(=O)N1CCN[C@H](C(=O)NCCN=[N+]=[N-])C1 ; CC(C)(C)OC(=O)N1CCN(C(=O)CNC(=O)C2(C(N)=O)CCN(C(=O)CCCC(C)(c3ccc(O)cc3)c3ccc(O)cc3)CC2)[C@H](C(=O)NCCN=[N+]=[N-])C1]

### 3.34 Synthesis of Intermediate 72

To a solution of tert-butyl(S)-3-((2-azidoethyl) carbamoyl) piperazine-1-carboxylate (10.1 g, 33.7 mmol, 1.5 eq) and compound 72A (11.5 g, 22.5 mmol, 1 eq) in pyridine (105 mL) was added EDCl (8.62 g, 45 mmol, 2 eq). The mixture was stirred at 20° C. for 1 h and concentrated under reduced pressure. The reaction was repeated and the combined residues were purified by column chromatography (SiO₂, EtOAc:MeOH) to give Intermediate 72 (23 g, 64.6% yield) as a white solid; LCMS [M+H]: 792.5; ¹H NMR (400 MHz, DMSO-d₆) δ 9.13 (s, 2H), 8.23 (brt, J=5.4 Hz, 1H), 7.97 (br s, 1H), 7.46 (br s, 1H), 7.24 (brs, 1H), 6.93 (d, J=8.6 Hz, 4H), 6.63 (d, J=8.6 Hz, 4H), 4.72 (br s, 1H), 4.35-4.13 (m, 2H), 3.92 (br dd, J=4.8, 16.8 Hz, 1H), 3.83-3.62 (m, 3H), 3.52-3.41 (m, 2H), 3.35 (br s, 2H), 3.21 (br d, J=11.8 Hz, 3H), 3.12 (brd, J=4.8 Hz, 2H), 2.24 (br t, J=7.2 Hz, 2H), 2.09-1.72 (m, 6H), 1.47 (s, 3H), 1.38 (s, 9H), 1.28-1.18 (m, 2H).

### C14. Synthesis of Intermediate 73

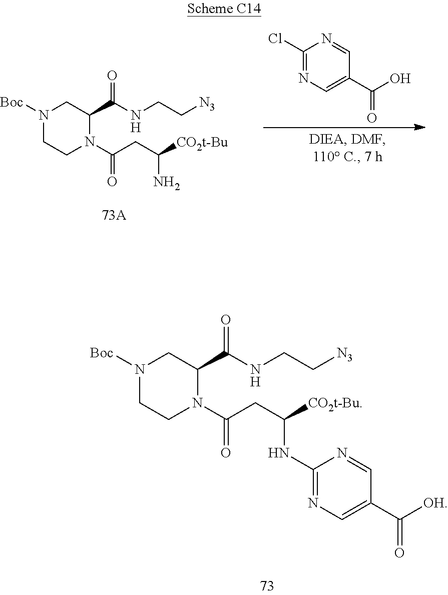 [SMILES: CC(C)(C)OC(=O)[C@@H](N)CC(=O)N1CCN(C(=O)OC(C)(C)C)C[C@H]1C(=O)NCCN=[N+]=[N-] ; O=C(O)c1cnc(Cl)nc1 ; CC(C)(C)OC(=O)[C@H](CC(=O)N1CCN(C(=O)OC(C)(C)C)C[C@H]1C(=O)NCCN=[N+]=[N-])Nc1ncc(C(=O)O)cn1]

### 3.35 Synthesis of Intermediate 73

To a solution of compound 73A (4.7 g, 10.0 mmol, 1 eq), 2-chloropyrimidine-5-carboxylic acid (1.59 g, 10.0 mmol, 1 eq) in DMF (50 mL) was added DIEA (5.23 mL, 30.0 mmol, 3 eq). The mixture was stirred at 110° C. for 7 h and the reaction mixture concentrated under reduced pressure. The residue was purified by column chromatography (SiO₂, DCM:MeOH) to give compound 8 (5.4 g, 46% yield) as a white oil; LCMS [M+H]: 592.2; ¹H NMR (400 MHz, DMSO-d₆) δ 12.82 (br s, 1H), 8.79-8.67 (m, 2H), 8.16 (br t, J=5.4 Hz, 1H), 8.04-7.83 (m, 1H), 4.80-4.68 (m, 2H), 4.38-4.13 (m, 1H), 3.82-3.64 (m, 2H), 3.54-3.41 (m, 1H), 3.37-3.33 (m, 2H), 3.19 (s, 1H), 3.17-2.91 (m, 3H), 2.87-2.77 (m, 1H), 1.43-1.31 (m, 18H).

### C15. Synthesis of Intermediate 74

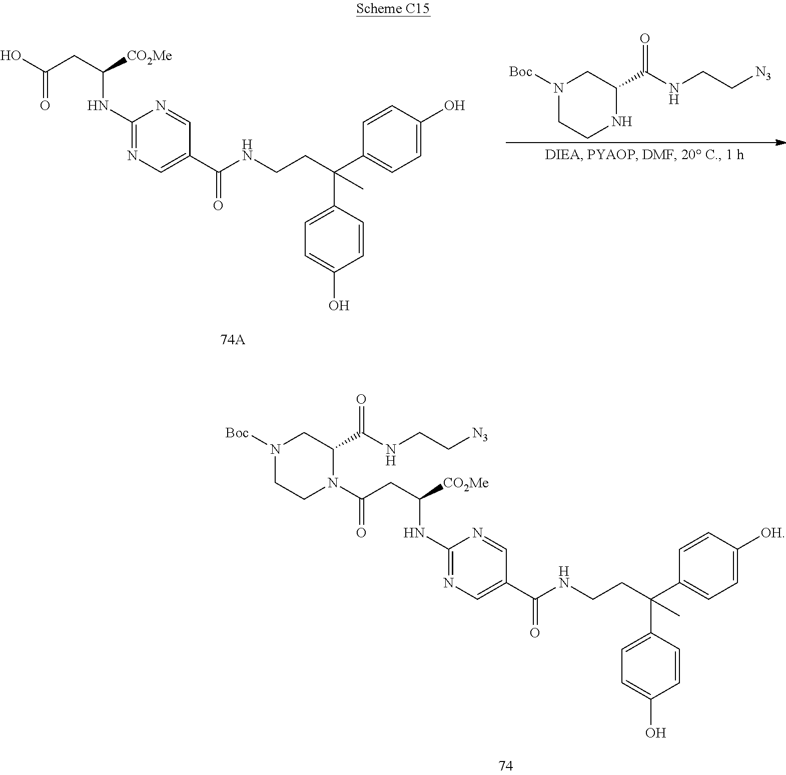 [SMILES: COC(=O)[C@H](CC(=O)O)Nc1ncc(C(=O)NCCC(C)(c2ccc(O)cc2)c2ccc(O)cc2)cn1 ; CC(C)(C)OC(=O)N1CCN[C@@H](C(=O)NCCN=[N+]=[N-])C1 ; COC(=O)[C@H](CC(=O)N1CCN(C(=O)OC(C)(C)C)C[C@@H]1C(=O)NCCN=[N+]=[N-])Nc1ncc(C(=O)NCCC(C)(c2ccc(O)cc2)c2ccc(O)cc2)cn1]

### 3.36 Synthesis of Intermediate 74

To a mixture of compound 74A (2.4 g, 4.72 mmol, 1 eq) and tert-butyl (R)-3-((2-azidoethyl) carbamoyl) piperazine-1-carboxylate (1.69 g, 5.66 mmol, 1.2 eq) in DMF (3 mL) was added DIEA (2.06 mL, 2.5 eq) and PyAOP (2.71 g, 5.19 mmol, 1.1 eq). The suspension was degassed under reduced pressure and purged with nitrogen 3 times. The mixture was stirred at 20° C. for 1 h, quenched by addition of H₂O (15 mL) at 20° C., diluted with H₂O (20 mL) and extracted with EtOAc (2×40 mL). The combined organic layers were washed with brine (20 mL), dried (Na₂SO₄), filtered and concentrated under reduced pressure. The residue was purified by column chromatography (SiO₂, DCM:MeOH) to give intermediate 74 (1.9 g, 51% yield) as a white solid.

### C16. Synthesis of Intermediate 75

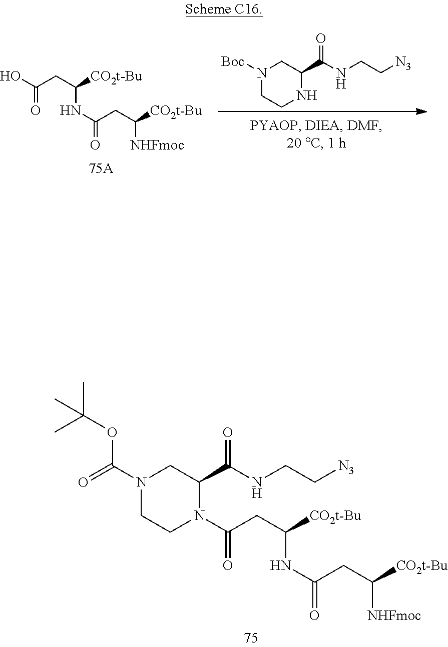

### 3.37 Synthesis of Intermediate 75

To a solution of tert-butyl(S)-3-((2-azidoethyl)carbamoyl)piperazine-1-carboxylate (20.0 g, 35.5 mmol, 1 eq) and compound 75A (21.5 g, 36.9 mmol, 1.0 eq) in DMF (200 mL) was added PyAOP (19.2 g, 36.9 mmol, 1.1 eq) and DIEA (11.7 mL, 67.0 mmol, 2 eq). The mixture was stirred at 20° C. for 1 h, the reaction mixture poured into water (300 mL) and extracted with EtOAc (3×100 mL). The combined organic phase was washed with a saturated NH₄Cl solution (100 mL), a saturated NaHCO₃ solution (100 mL) and brine (100 mL), dried (Na₂SO₄) and concentrated under reduced pressure. The residue was purified by column chromatography (SiO₂, petroleum ether:EtOAc) to give Intermediate 75 (18 g, 62% yield) as a yellow oil; LCMS [M+H]: 863.6; ¹H NMR (400 MHz, METHANOL-d₄) δ 7.80 (br d, J=7.4 Hz, 2H), 7.68 (br d, J=7.4 Hz, 2H), 7.44-7.35 (m, 2H), 7.31 (s, 2H), 4.88 (br s, 2H), 4.74-4.61 (m, 1H), 4.54-4.17 (m, 5H), 3.93-3.73 (m, 2H), 3.61-3.48 (m, 1H), 3.38 (br d, J=5.2 Hz, 3H), 3.29-2.65 (m, 7H), 1.52-1.37 (m, 27H).

### C17. Synthesis of Intermediate 76

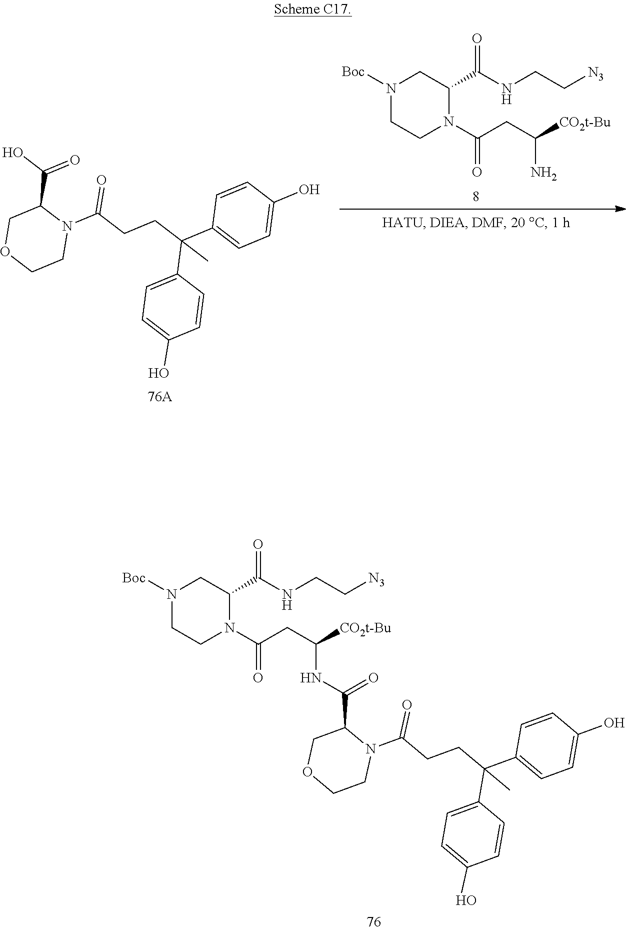 [SMILES: CC(CCC(=O)N1CCOC[C@H]1C(=O)O)(c1ccc(O)cc1)c1ccc(O)cc1 ; CC(C)(C)OC(=O)[C@@H](N)CC(=O)N1CCN(C(=O)OC(C)(C)C)C[C@@H]1C(=O)NCCN=[N+]=[N-] ; CC(C)(C)OC(=O)[C@H](CC(=O)N1CCN(C(=O)OC(C)(C)C)C[C@@H]1C(=O)NCCN=[N+]=[N-])NC(=O)[C@@H]1COCCN1C(=O)CCC(C)(c1ccc(O)cc1)c1ccc(O)cc1]

### 3.38 Synthesis of Intermediate 76

To a solution of compound 76A (0.50 g, 1.25 mmol, 1 eq) and tert-butyl (R)-4-((S)-3-amino-4-(tert-butoxy)-4-oxobutanoyl)-3-((2-azidoethyl) carbamoyl) piperazine-1-carboxylate (588 mg, 1.25 mmol, 1 eq) in DMF (3 mL) was added HATU (476 mg, 1.25 mmol, 1 eq) and DIEA (654 μL, 3.76 mmol, 3 eq) at 20° C. The reaction mixture was stirred at 20° C. for 1 h, diluted with EtOAc (4 mL), washed with aq. NH₄Cl (2 mL), aq. Na₂CO₃ (2 mL) and brine (3 ml), dried (Na₂SO₄), filtered and concentrated under reduced pressure. The residue was purified by column chromatography (SiO₂, petroleum ether:EtOAc) to give Intermediate 76 (1.0 g, 94% yield) as a white solid; LCMS [M+H]: 851.4.

### C18. Synthesis of Intermediate 77

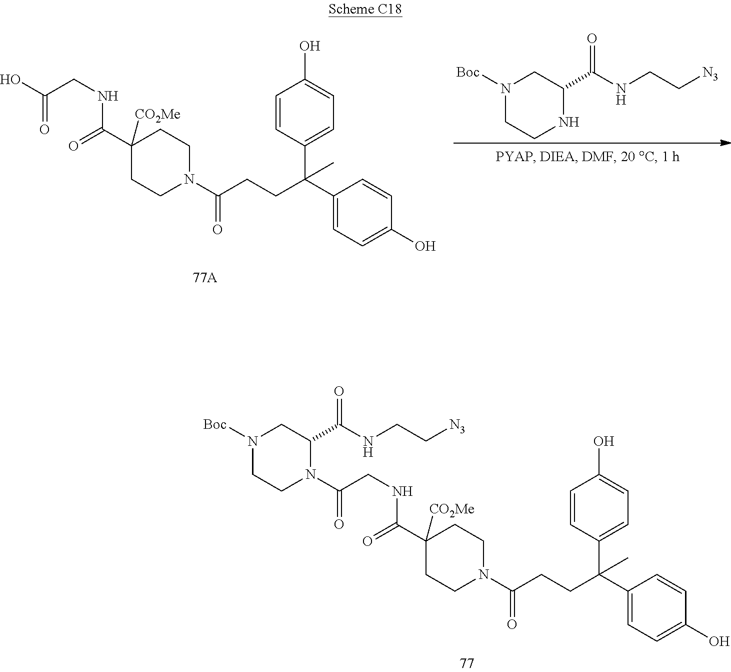 [SMILES: COC(=O)C1(C(=O)NCC(=O)O)CCN(C(=O)CCC(C)(c2ccc(O)cc2)c2ccc(O)cc2)CC1 ; CC(C)(C)OC(=O)N1CCN[C@@H](C(=O)NCCN=[N+]=[N-])C1 ; COC(=O)C1(C(=O)NCC(=O)N2CCN(C(=O)OC(C)(C)C)C[C@@H]2C(=O)NCCN=[N+]=[N-])CCN(C(=O)CCC(C)(c2ccc(O)cc2)c2ccc(O)cc2)CC1]

### 3.39 Synthesis of Intermediate 77

To a mixture of tert-butyl (R)-3-((2-azidoethyl)carbamoyl)piperazine-1-carboxylate (450 mg, 1.51 mmol, 1 eq) and compound 77A (773 mg, 1.51 mmol, 1 eq) in DMF (5 mL) was added DIEA (657 μL, 3.77 mmol, 2.5 eq) and PyAOP (865 mg, 1.66 mmol, 1.1 eq) at 20° C. under a nitrogen atmosphere. The mixture was stirred at 20° C. for 1 h, poured into water (80 mL) and the aqueous phase extracted with EtOAc (3×120 mL). The combined organic phase was washed with brine (2×20 mL), dried (Na₂SO₄), filtered and concentrated under reduced pressure. The crude product was purified by column chromatography (DCM:MeOH) to give Intermediate 77 (850 mg, 71% yield) as white solid; ¹H NMR (400 MHz, METHANOL-d₄) δ 7.02 (br d, J=8.4 Hz, 5H), 6.69 (br d, J=7.6 Hz, 4H), 4.10 (q, J=7.0 Hz, 4H), 3.94-3.84 (m, 1H), 3.83-3.67 (m, 5H), 3.65-3.53 (m, 1H), 3.44-3.36 (m, 4H), 3.33-3.30 (m, 8H), 3.28-3.10 (m, 3H), 2.37-2.24 (m, 2H), 2.15-2.05 (m, 4H), 1.46 (s, 9H).

Compounds in table 8.4 were prepared by procedures depicted in scheme C18.

TABLE 8.4

| Compound | Structure | m/z |
|---|---|---|
| 77.1 | 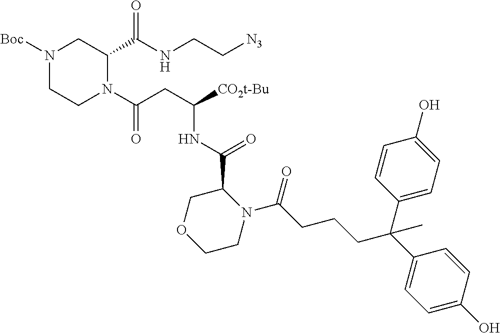 [SMILES: CC(C)(C)OC(=O)[C@H](CC(=O)N1CCN(C(=O)OC(C)(C)C)C[C@@H]1C(=O)NCCN=[N+]=[N-])NC(=O)[C@@H]1COCCN1C(=O)CCCC(C)(c1ccc(O)cc1)c1ccc(O)cc1] | [M + H]: 865.3 |
| | 8 | |

### C19. Synthesis of Intermediate 78

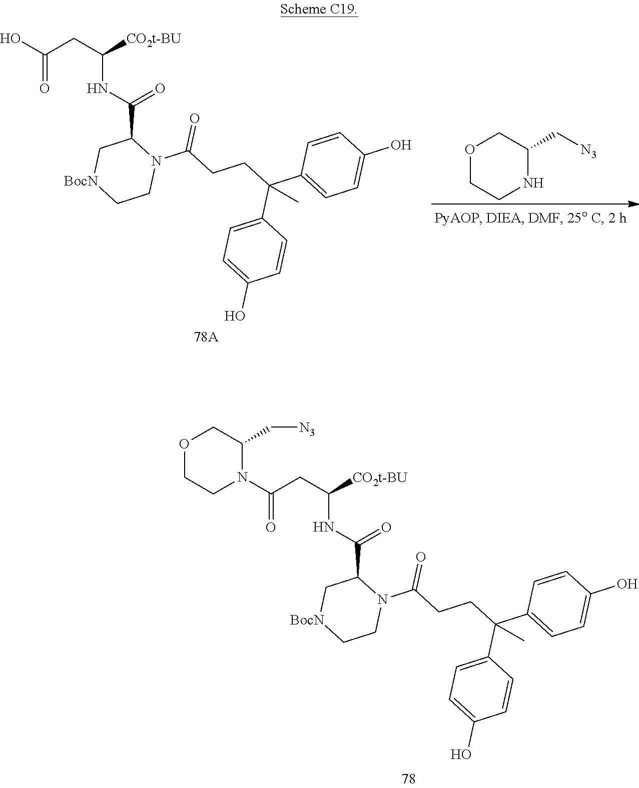

### 3.40 Synthesis of Intermediate 78

To a solution of(S)-3-(azidomethyl) morpholine (500 mg, 3.52 mmol, 1 eq) and compound 78A (1.88 g, 2.81 mmol, 0.8 eq) in DMF (5 mL) was added PyAOP (1.47 g, 2.81 mmol, 0.8 eq) and DIEA (1.84 mL, 10.6 mmol, 3 eq). The mixture was stirred at 25° C. for 2 h and extracted with EtOAc (3× 20 mL). The combined organics were washed with brine (20 mL), dried (Na₂SO₄) and concentrated under reduced pressure. The residue was purified by flash chromatography (SiO₂, petroleum ether:EtOAc) to give Intermediate 78 (710 mg, 25% yield) as a yellow solid; ¹H NMR (400 MHz, DMSO-d₆) δ 9.16 (d, J=2.3 Hz, 1H), 8.03-8.29 (m, 1H), 7.95 (s, 1H), 6.95 (br d, J=8.4 Hz, 4H), 6.64 (br d, J=8.4 Hz, 4H), 4.83 (brs, 1H), 4.32-4.60 (m, 2H), 4.09-4.30 (m, 1H), 3.69-3.96 (m, 3H), 3.38-3.62 (m, 4H), 3.23-3.29 (m, 1H), 3.08-3.19 (m, 1H), 2.97-3.08 (m, 1H), 2.86-2.92 (m, 3H), 2.73 (s, 2H), 2.20 (br d, J=5.0 Hz, 2H), 2.02-2.15 (m, 2H), 1.44-1.54 (m, 3H), 1.36 (d, J=1.6 Hz, 18H).

### C20. Synthesis of Intermediate 79

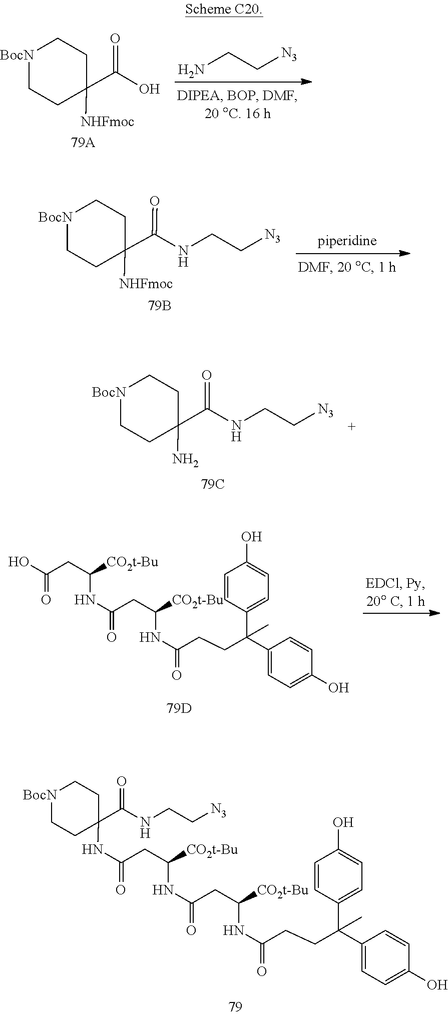

### 3.41 Synthesis of Compound 79B

To a mixture of 2-azidoethan-1-amine (1.31 g, 10.7 mmol, 1 eq) in DMF (40 mL) was added DIEA (2.1 mL, 11.8 mmol, 1.1 eq), the mixture stirred at 20° C. for 10 minutes, compound 79A (5.0 g, 10.7 mmol, 1 eq) and BOP (4.74 g, 10.7 mmol, 1 eq) added at 20° C. and the reaction stirred at 20° C. for 16 h. The reaction mixture was poured into water (50 mL), the aqueous phase extracted with EtOAc (3×200 mL), the combined organics washed with brine (2×100 mL), dried (Na₂SO₄), filtered and concentrated under reduced pressure. The residue was purified by column chromatography (SiO₂, petroleum ether:EtOAc) to give compound 79B (11.8 g, 69% yield) as a white solid; ¹H NMR (400 MHz, DMSO-d₆) δ 7.95-7.86 (m, 2H), 7.82 (brs, 1H), 7.74 (br d, J=7.2 Hz, 2H), 7.50 (br s, 1H), 7.47-7.28 (m, 4H), 4.34-4.14 (m, 3H), 3.64 (br d, J=12.0 Hz, 2H), 3.32-3.15 (m, 4H), 3.01 (br s, 2H), 1.89 (br s, 2H), 1.77 (br t, J=10.2 Hz, 2H), 1.40 (s, 9H).

### 3.42 Synthesis of Compound 79C

To a mixture of compound 79B (5.4 g, 10.1 mmol, 1 eq) in DMF (100 mL) was added piperidine (4.99 mL, 50.5 mmol, 5 eq) in one portion at 20° C. The mixture was stirred at 20° C. for 1 h and concentrated under reduced pressure. The residue was purified by column chromatography (SiO₂, petroleum ether:EtOAc) to give compound 79C (4.5 g, 71% yield) as a light yellow solid; ¹H NMR (400 MHz, CHLOROFORM-d) δ 8.04 (br s, 1H), 4.08-3.90 (m, 2H), 3.49-3.40 (m, 4H), 3.06 (br t, J=12.2 Hz, 2H), 2.16 (ddd, J=4.6, 11.8, 13.8 Hz, 2H), 1.47 (s, 9H), 1.42-1.32 (m, 2H).

### 3.43 Synthesis of Intermediate 79

To a solution of compound 79C (160 mg, 512 μmol, 1 eq) in pyridine (3.5 mL) was added compound 79D (354 mg, 563 μmol, 1.1 eq) and EDCl (196 mg, 1.02 mmol, 2 eq) at 20° C. for 1 h. The reaction mixture was concentrated under reduced pressure and purified by column chromatography (SiO₂, petroleum ether:EtOAc) to give Intermediate 79 (360 mg, 76% yield) as a white solid; ¹H NMR (400 MHz, CHLOROFORM-d) δ 7.14-6.83 (m, 7H), 6.81-6.62 (m, 5H), 4.90-4.57 (m, 2H), 3.94-3.77 (m, 2H), 3.54-3.31 (m, 4H), 3.12-2.98 (m, 2H), 2.76-2.58 (m, 3H), 2.48-2.27 (m, 3H), 2.06-1.90 (m, 6H), 1.70 (br s, 4H), 1.50-1.38 (m, 27H).

### C21. Synthesis of Intermediate 80

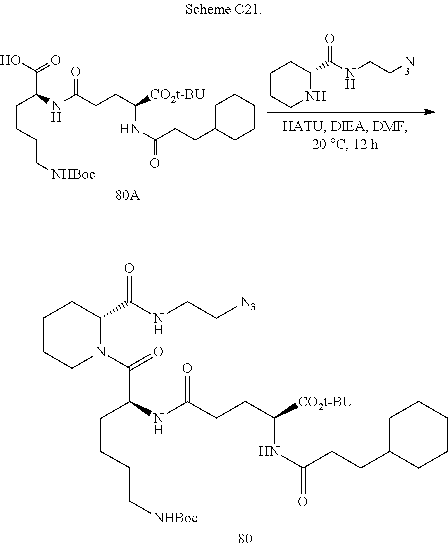

### 3.45 Synthesis of Intermediate 80

To a solution of (R)—N-(2-azidoethyl) morpholine-3-carboxamide (825 mg, 2.63 mmol, 1 eq, TFA) and compound 80A (1.5 g, 2.63 mmol, 1 eq) in DMF (15 mL) was added HATU (1.00 g, 2.63 mmol, 1 eq) and DIEA (1.38 mL, 7.90 mmol, 3 eq). The mixture was stirred at 20° C. for 12 h, diluted with EtOAc (20 mL) and extracted with H₂O (10 mL). The combined organic layers were washed with brine (9 mL), dried (Na₂SO₄), filtered and concentrated under reduced pressure. The residue was purified by column chromatography (SiO₂, DCM:MeOH) to give Intermediate 80 (1.75 g, 89% yield) as a white solid; LCMS; [M+H]: 751.5.

Compounds in table 8.5 were prepared by procedures depicted in scheme C21.

TABLE 8.5

| Compound | Structure | m/z |
|---|---|---|
| 80.1 | 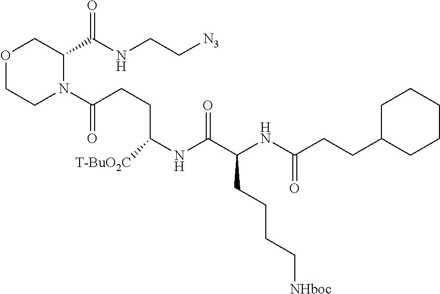 | [M + H]: 751.4 |

### C22. Synthesis of Intermediate 81

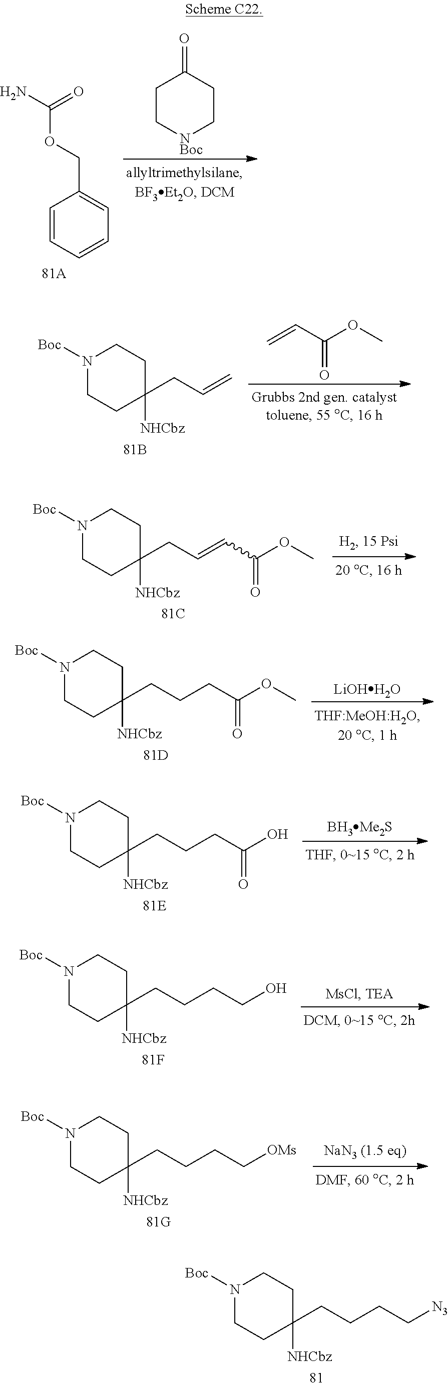

### 3.46 Synthesis of Compound 81B

To a solution of tert-butyl 4-oxopiperidine-1-carboxylate (100 g, 502 mmol, 1 eq), compound 81A (91.0 g, 602 mmol, 1.2 eq) and allyl (trimethyl) silane (96.1 mL, 602 mmol, 1.2 eq) in DCM (1000 mL) was added BF₃·Et₂O (74.3 mL, 602 mmol, 1.2 eq) dropwise at 0° C. The solution was stirred at 0° C. for 40 min and 30° C. for 16 h. The reaction was concentrated under reduced pressure, the residue diluted with EtOAc (1000 mL), washed with NaHCO₃ (300 mL) and brine (200 mL), the organics dried (Na₂SO₄), filtered and concentrated under reduced pressure. The residue was purified by column chromatography (SiO₂, Petroleum ether:EtOAc) to give compound 81B (89 g, 23% yield) as a light-yellow oil.

### 3.47 Synthesis of Compound 81C

To a solution of compound 81B (63 g, 168 mmol, 1 eq) in toluene (630 mL) was added methyl acrylate (76 mL, 841 mmol, 5 eq) and Grubbs 2^{nd} generation catalyst (14.3 g, 16.8 mmol, 0.1 eq). The mixture was stirred at 55° C. for 16 h and concentrated under reduced pressure. The residue was purified by column chromatography (SiO₂, petroleum ether:EtOAc) to give compound 81C (54 g, 71% yield) as a colorless oil.

### 3.48 Synthesis of Compound 81D

To a solution of compound 81C (54 g, 125 mmol, 1 eq) in EtOAc (48 mL) was added PtO₂ (18.4 g, 81.2 mmol, 0.65 eq) at 20° C. The mixture was stirred at 20° C. for 16 h under a hydrogen atmosphere (15 psi). The mixture was filtered, and the filtrate concentrated under reduced pressure to give compound 81D (54 g, 38% yield) as an oil.

### 3.49 Synthesis of Compound 81E

To a solution of compound 81D (54 g, 124 mmol, 1 eq) in THF (225 mL), MeOH (225 mL) and H₂O (160 mL) was added LiOH·H₂O (20.9 g, 497 mmol, 4 eq). The mixture was stirred at 20° C. for 2 h, adjusted to pH=4 with a saturated citric acid solution, extracted with EtOAc (3×300 mL), the organic phase washed with brine (150 mL), dried (Na₂SO₄), filtered and concentrated to give compound 81E (54 g, 83% yield) as a white solid.

### 3.50 Synthesis of Compound 81F

To a solution of compound 81E (34 g, 81 mmol, 1 eq) in THF (340 mL) was added BH₃-Me₂S (28.3 mL, 283 mmol, 3.5 eq) dropwise at 0° C. The mixture was stirred at 15° C. for 2 h, the reaction quenched with an aqueous ammonium chloride (30 mL) and extracted with EtOAc (3×300 mL). The organics were washed with brine (150 mL), dried (Na₂SO₄), filtered and concentrated under reduced pressure. The residue was purified by column chromatography (SiO₂, DCM:MeOH) to give compound 81F (20 g, 55% yield) as a brown oil.

### 3.51 Synthesis of Compound 81G

To a solution of compound 81F (21 g, 51.7 mmol, 1 eq) in DCM (210 mL) was added TEA (21.6 mL, 155 mmol, 3 eq) and MsCl (6.00 mL, 77 mmol, 1.5 eq) dropwise at 0° C. The mixture was stirred at 15° C. for 2 h. The mixture was quenched with aq. NH₄Cl (50 mL) and extracted with DCM (3×200 mL). The organic phase was washed with brine (100 mL), dried (Na₂SO₄), filtered and concentrated to give compound 81G (25 g, crude) as a brown oil.

### 3.52 Synthesis of Intermediate 81

To a solution of compound 81G (35.7 g, 73.7 mmol, 1 eq) in DMF (350 mL) was added NaN₃ (7.18 g, 111 mmol, 1.5 eq). The mixture was stirred at 60° C. for 2 h, diluted with H₂O (500 mL), extracted with EtOAc (3×200 mL), the organic layers washed with brine (3×100 mL), dried (Na₂SO₄), filtered and concentrated under reduced pressure. (The aqueous phase was poured into H₂O (1000 mL) and NaClO (1000 mL) added slowly, the mixture was stirred at 20° C. for 16 h and discarded). The residue was purified by column chromatography (SiO₂, Petroleum ether:EtOAc) to give Intermediate 81 (21 g, 63% yield) as a white solid; ¹H NMR (400 MHz, CDCl₃) δ 7.33-7.22 (m, 5H), 5.05-4.93 (s, 2H), 4.51-4.39 (s, 1H), 3.80-3.62 (s, 2H), 3.23-3.13 (m, 2H), 3.03-2.87 (m, 2H), 1.97-1.85 (m, 2H), 1.73-1.61 (m, 2H), 1.54-1.47 (m, 2H), 1.47-1.40 (m, 2H), 1.38 (s, 9H), 1.31-1.19 (m, 2H).

### C23. Synthesis of Intermediate 82

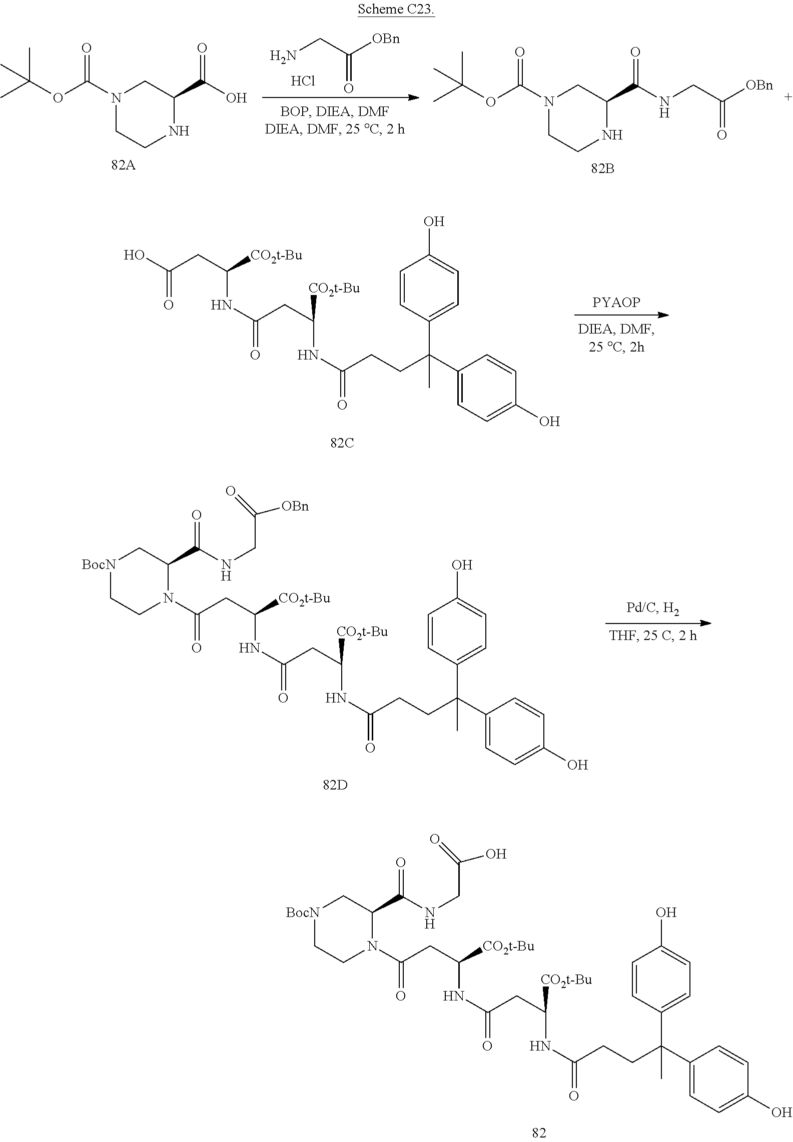

### 3.53 Synthesis of Compound 82B

To a solution of benzyl glycinate (4.82 g, 23.9 mmol, 1.1 eq) in DMF (50 mL) was added DIEA (15.1 mL, 86.9 mmol, 4 eq), compound 82A (5.0 g, 21.7 mmol, 1 eq) and BOP (10.6 g, 23.9 mmol, 1.1 eq). The mixture was stirred at 25° C. for 2 h, poured into water (50 mL) and the aqueous phase extracted with EtOAc (2×50 mL). The combined organic layers were washed with brine (50 mL), dried (Na₂SO₄), filtered and concentrated under reduced pressure. The residue was purified by prep-HPLC (column: Phenomenex luna C18 (250*70 mm, 10 μm); mobile phase: [water (TFA)-ACN]; gradient: 15%-50% B over 20 min) to give compound 82B (1.5 g, 14% yield, TFA) as a white solid.

### 3.54 Synthesis of Compound 82D

To a solution of compound 82C (1.50 g, 2.38 mmol, 1 eq) in DMF (18 mL) was added DIEA (1.25 mL, 7.15 mmol, 3 eq) and compound 82B (0.9 g, 2.38 mmol, 1 eq), followed by PyAOP (1.37 g, 2.62 mmol, 1.1 eq). The mixture was stirred at 25° C. for 3 h in 2 batches. The reaction mixture was poured into H₂O (50 mL), extracted with EtOAc (3×50 mL), combined organics washed with brine (100 mL), dried (Na₂SO₄) and concentrated under reduced pressure. The residue was purified by flash chromatography (SiO₂, petroleum ether:EtOAc) to give compound 82D (1.3 g, 28% yield, 75% purity) as a white solid; LCMS: [M+H]: 988.7; ¹H NMR (400 MHz, CDCl₃) δ 8.03 (s, 2H), 7.33 (br d, J=2.1 Hz, 3H), 6.95 (d, J=7.4 Hz, 4H), 6.71-6.65 (m, 4H), 5.13 (br s, 2H), 4.94-4.65 (m, 3H), 4.56-4.31 (m, 1H), 4.26-4.12 (m, 1H), 3.99-3.77 (m, 2H), 3.65-3.51 (m, 1H), 3.45-3.27 (m, 1H), 3.21-3.02 (m, 2H), 2.97 (s, 6H), 2.89 (s, 7H), 2.76-2.61 (m, 2H), 2.48-2.29 (m, 2H), 2.05-1.91 (m, 2H), 1.87-1.61 (m, 2H), 1.52-1.43 (m, 17H).

### 3.55 Synthesis of Intermediate 82

To a solution of Pd/C (2.7 g, 10% purity) in THF (27 mL) was added compound 82D (1.3 g, 1.32 mmol, 1 eq), and the mixture stirred at 25° C. for 2 h under a hydrogen atmosphere (15 psi). The mixture was filtered and concentrated under reduced pressure to give crude Intermediate 82 as a white solid; LCMS [M+H]: 898.6; ¹H NMR (400 MHz, CDCl₃) δ 8.03 (s, 2H), 6.69 (br s, 2H), 5.66-5.56 (m, 1H), 4.91-4.77 (m, 1H), 4.04-3.93 (m, 3H), 3.76 (t, J=6.4 Hz, 2H), 2.97 (s, 6H), 2.89 (s, 6H), 2.80-2.65 (m, 2H), 2.31-2.24 (m, 2H), 2.00-1.84 (m, 7H), 1.53-1.43 (m, 23H).

### C24. Synthesis of Intermediate 83

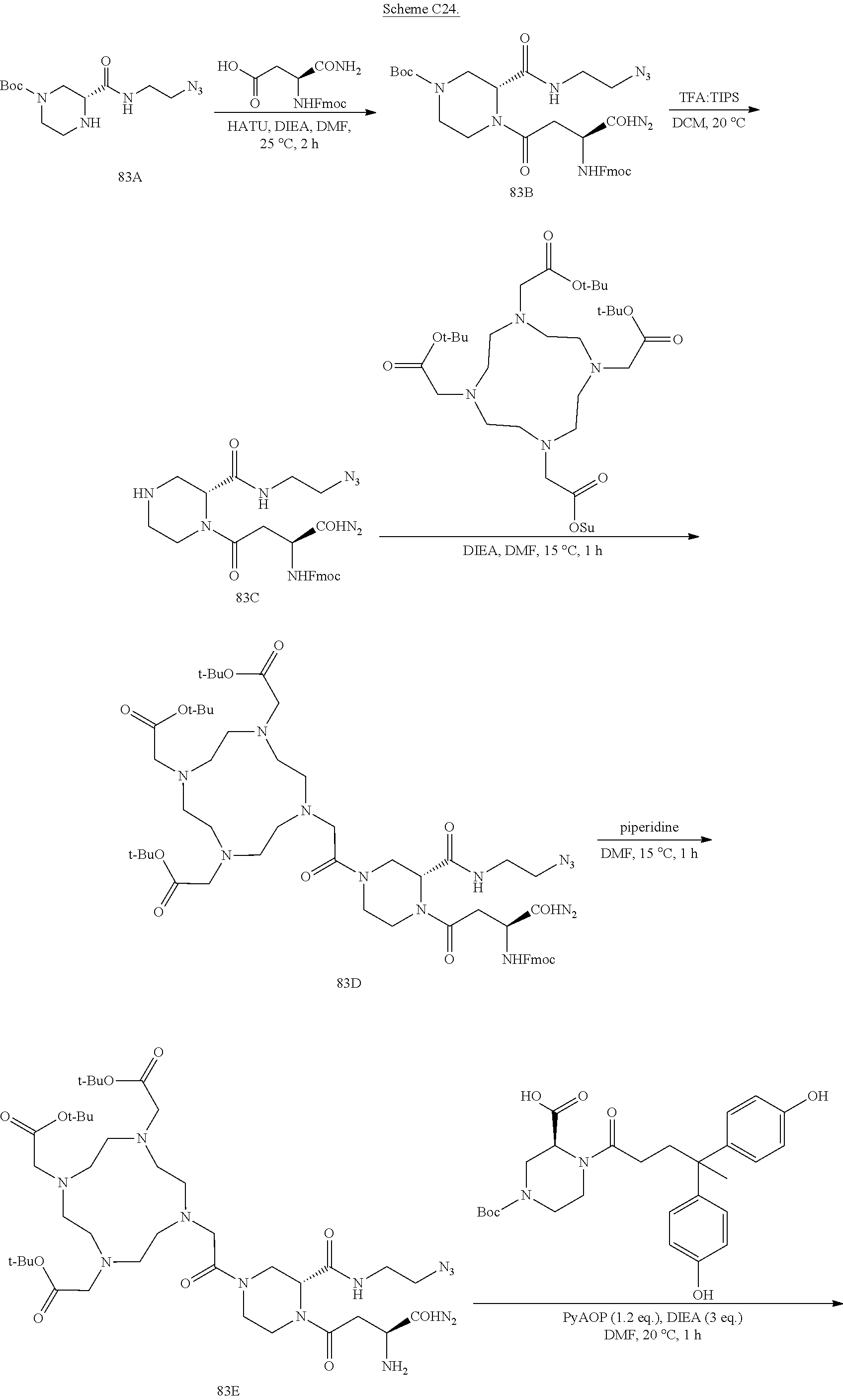

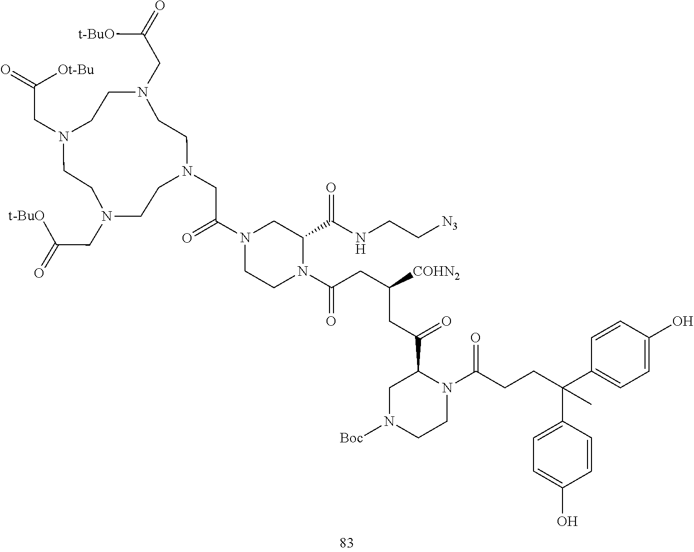

### 3.56 Synthesis of Compound 83B

To a solution of compound 83A (8.92 g, 29.9 mmol, 2 eq) and(S)-3-((((9H-fluoren-9-yl)methoxy)carbonyl)amino)-4-amino-4-oxobutanoic acid (5.3 g, 15.0 mmol, 1 eq) in DMF (25 mL) was added HATU (6.82 g, 18 mmol, 1.2 eq) and DIEA (7.82 mL 44.9 mmol, 3 eq) at 25° C. The mixture was stirred at 25° C. for 2 h, the reaction extracted with DCM (3×40 mL) and the combined organic layers washed with brine (30 mL), dried (Na₂SO₄), filtered and concentrated under reduced pressure. The residue was purified by prep-HPLC (column: Welch Xtimate C18 180*70 mm #10 μm; mobile phase: [water (NH₄HCO₃)-ACN]; B %: 30%-60%, 15 min) to give compound 83B (3.8 g, 40% yield); LCMS [M+H]: 635.4; ¹H NMR (400 MHz, DMSO-d₆) δ 8.02-8.12 (m, 1H), 7.86-7.92 (m, 2H), 7.67-7.74 (m, 2H), 7.36-7.45 (m, 3H), 7.26-7.36 (m, 3H), 7.09 (br s, 1H), 4.71-4.88 (m, 1H), 4.16-4.43 (m, 5H), 3.64-3.84 (m, 2H), 3.35-3.53 (m, 2H), 3.02-3.22 (m, 2H), 2.65-2.85 (m, 2H), 1.31-1.45 (m, 9H).

### 3.57 Synthesis of Compound 83C

To a solution of compound 83B (1.95 g, 3.07 mmol, 1 eq) in DCM (16 mL) was added TFA (4 mL) and TIPS (6.31 mL, 30.7 mmol, 10 eq) at 20° C. The mixture was stirred at 20° C. for 1 h and concentrated under reduced pressure to give crude compound 83C (1.99 g) as a white solid; LCMS [M+H]: 535.3.

### 3.58 Synthesis of Compound 83D

To a solution of compound 83C (2.76 g, 4.26 mmol, 1 eq, TFA) in DMF (30 mL) was added tri-tert-butyl 2,2′,2″-(10-(2-((2,5-dioxopyrrolidin-1-yl)oxy)-2-oxoethyl)-1,4,7,10-tetraazacyclododecane-1,4,7-triyl)triacetate (7.13 g, 10.6 mmol, 2.5 eq) and DIEA (1.48 mL, 8.51 mmol, 2 eq) at 15° C. The mixture was stirred at 15° C. for 1 h, filtered, concentrated under reduced pressure and the residue purified by column chromatography (SiO₂, DCM:MeOH) to give compound 83D (4.2 g, 91% yield) as a white solid; LCMS [M+H]: 1089.6.

### 3.59 Synthesis of Compound 83E

To a solution of compound 83D (4.2 g, 3.86 mmol, 1 eq) in DMF (20 mL) was added piperidine (400 μL, 4.05 mmol, 1.05 eq). The mixture was stirred at 15° C. for 1 h, concentrated under reduced pressure and the residue purified by column chromatography (SiO₂, DCM:MeOH) to give compound 83E (2.0 g, 60% yield) as a white solid; LCMS [M+H]: 867.5.

### 3.61 Synthesis of Intermediate 83

To a solution of(S)-1-(4,4-bis(4-hydroxyphenyl)pentanoyl)-4-(tert-butoxycarbonyl) piperazine-2-carboxylic acid (831 mg, 1.67 mmol, 0.85 eq) and compound 83E (1.7 g, 1.96 mmol, 1 eq) in DMF (20 mL) was added PYAOP (1.23 g, 2.4 mmol, 1.2 eq) and DIEA (1.02 mL, 5.88 mmol, 3 eq) at 20° C. The mixture was stirred at 20° C. for 1 h, diluted with water (15 mL) and extracted with EtOAc (3× 20 mL). The combined organic layers were washed with brine (10 mL), dried (Na₂SO₄), filtered and concentrated under reduced pressure. The residue was purified by column chromatography (SiO₂, Petroleum ether:EtOAc) to give Intermediate 83 (2.1 g, 79% yield) as a white solid; LCMS [M+H]: 1347.9.

### C25. Synthesis of Compound 84

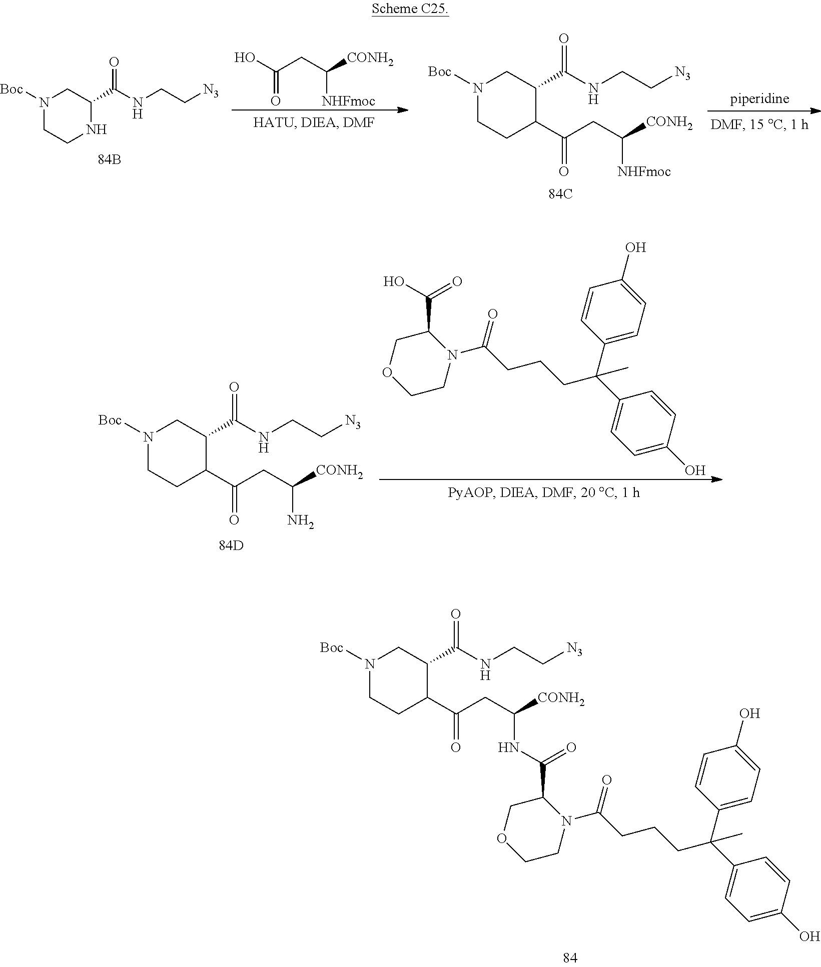

### 3.62 Synthesis of Compound 84C

To a solution of compound 84B (4.13 g, 13.8 mmol, 1 eq) and(S)-3-((((9H-fluoren-9-yl)methoxy)carbonyl)amino)-4-amino-4-oxobutanoic acid (4.9 g, 13.8 mmol, 1 eq) in DMF (100 mL) was added HATU (6.31 g, 16.6 mmol, 1.2 eq) and DIEA (7.23 mL, 41.5 mmol, 3 eq) at 20° C. The mixture was stirred at 20° C. for 1 h and directly by prep-HPLC (column: Welch Xtimate C18 250*100 mm #10 μm; mobile phase: [water (NH4HCO3)-ACN]; B %: 25%-55%, 20 min) directly to give compound 84C (2.5 g, 28% yield) as a white solid; LCMS [M+H]:635.4.

### 3.63 Synthesis of Compound 84D

To a solution of compound 84C (2.0 g, 3.2 mmol, 1 eq) in DMF (20 mL) was added piperidine (327 μL, 3.31 mmol, 1.05 eq) at 15° C. The mixture was stirred at 15° C. for 1 h, concentrated under reduced pressure and purified by column chromatography (SiO₂, DCM:MeOH) to give compound 84D (1.07 g, 82% yield) as a white solid; LCMS [M+H]:413.2.

### 3.64 Synthesis of Intermediate 84

To a solution of(S)-4-(5,5-bis(4-hydroxyphenyl)hexanoyl)morpholine-3-carboxylic acid (1.56 g, 3.78 mmol, 1.2 eq) and compound 84D (1.3 g, 3.15 mmol, 1 eq) in DMF (20 mL) was added DIEA (1.65 mL, 9.46 mmol, 3 eq) and PYAOP (1.97 g, 3.78 mmol, 1.2 eq) at 20° C. The mixture was stirred at 20° C. for 1 h, water (15 mL) added, extracted with EtOAc (3×20 mL), combined organics washed with brine (10 mL), dried (Na₂SO₄), filtered and concentrated under reduced pressure. The residue was purified by column chromatography (SiO₂, Petroleum ether:EtOAc) to give crude Intermediate 84 (5.0 g) as a white solid that was used directly in the next reaction; LCMS [M+H]: 808.5.

### C26. Synthesis of Intermediate 85

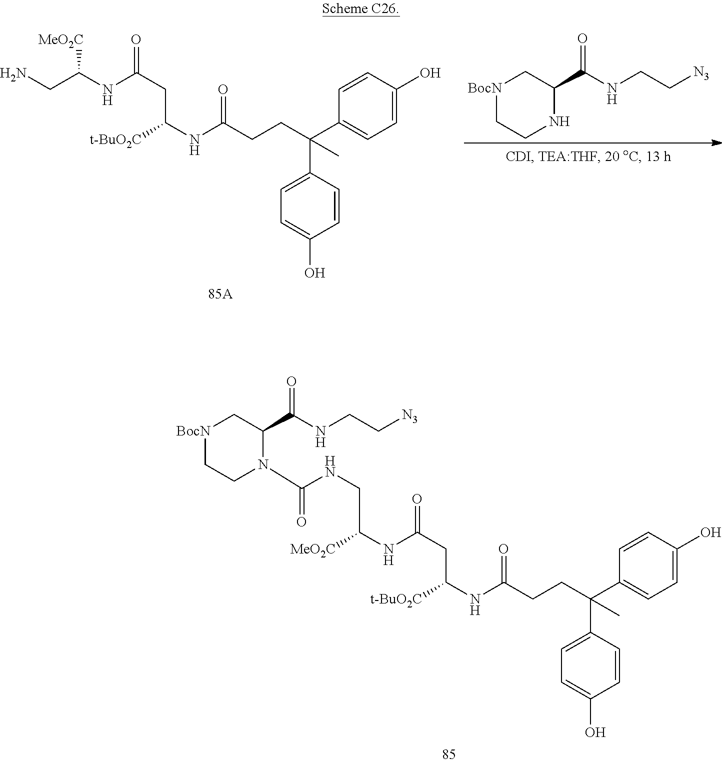

### 3.65 Synthesis of Intermediate 85

To a mixture of tert-butyl(S)-3-((2-azidoethyl)carbamoyl)piperazine-1-carboxylate (631 mg, 1.06 mmol, 1 eq) in DMF (2 mL) was added TEA (147 μL, 1.06 mmol, 1 eq) and CDI (172 mg, 1.06 mmol, 1 eq) and the mixture stirred at 20° C. for 1 h. Compound 85A (590 mg, 1.06 mmol, 1 eq) was added in one portion at 20° C. under a nitrogen atmosphere and the mixture stirred at 20° C. for 12 h. The reaction mixture was poured into water (50 mL), the aqueous phase extracted with EtOAc (5×100 mL), the combined organic phase washed with brine (2×50 mL), dried (Na₂SO₄), filtered and concentrated under reduced pressure. The crude product was purified by column chromatography (SiO₂, DCM:MeOH) to give Intermediate 85 (1.2 g, 76% yield, 59% purity) as a light yellow solid; LCMS [M+H]:882.4; ¹H NMR (400 MHz, DMSO-d₆) δ 9.14 (br s, 2H), 8.18 (br d, J=7.2 Hz, 1H), 8.09 (s, 1H), 8.03-7.86 (m, 2H), 7.17 (s, 2H), 6.93 (br d, J=8.6 Hz, 4H), 6.64 (d, J=8.6 Hz, 4H), 4.45-4.35 (m, 1H), 4.32-4.25 (m, 1H), 3.57 (s, 3H), 3.49-3.35 (m, 4H), 3.30-3.16 (m, 5H), 3.08 (br d, J=9.4 Hz, 2H), 2.56 (br s, 1H), 2.47-2.39 (m, 1H), 2.22-2.11 (m, 2H), 1.91-1.79 (m, 2H), 1.45 (s, 3H), 1.42-1.31 (m, 18H).

### D1. Synthesis of Compound 100

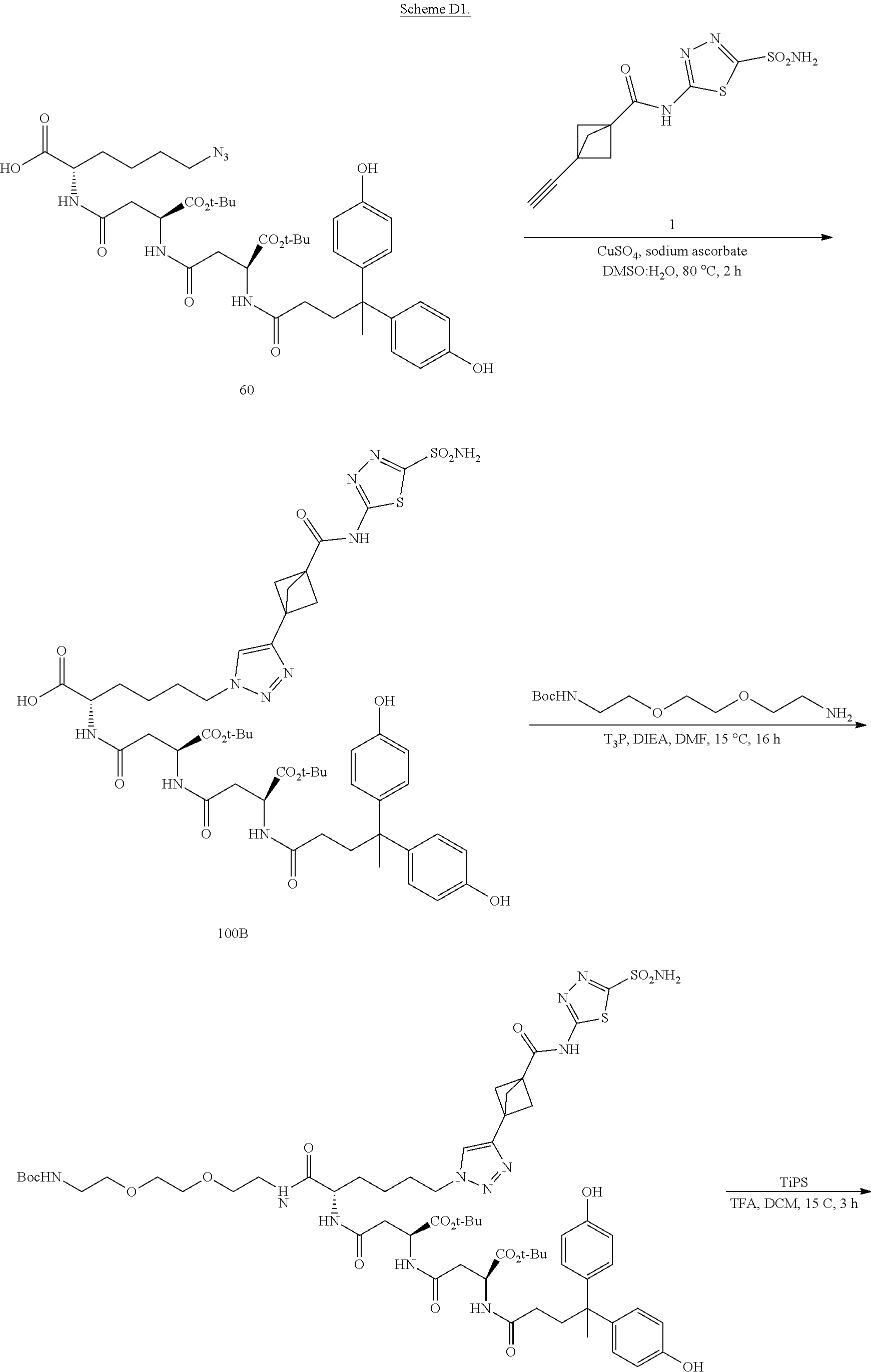

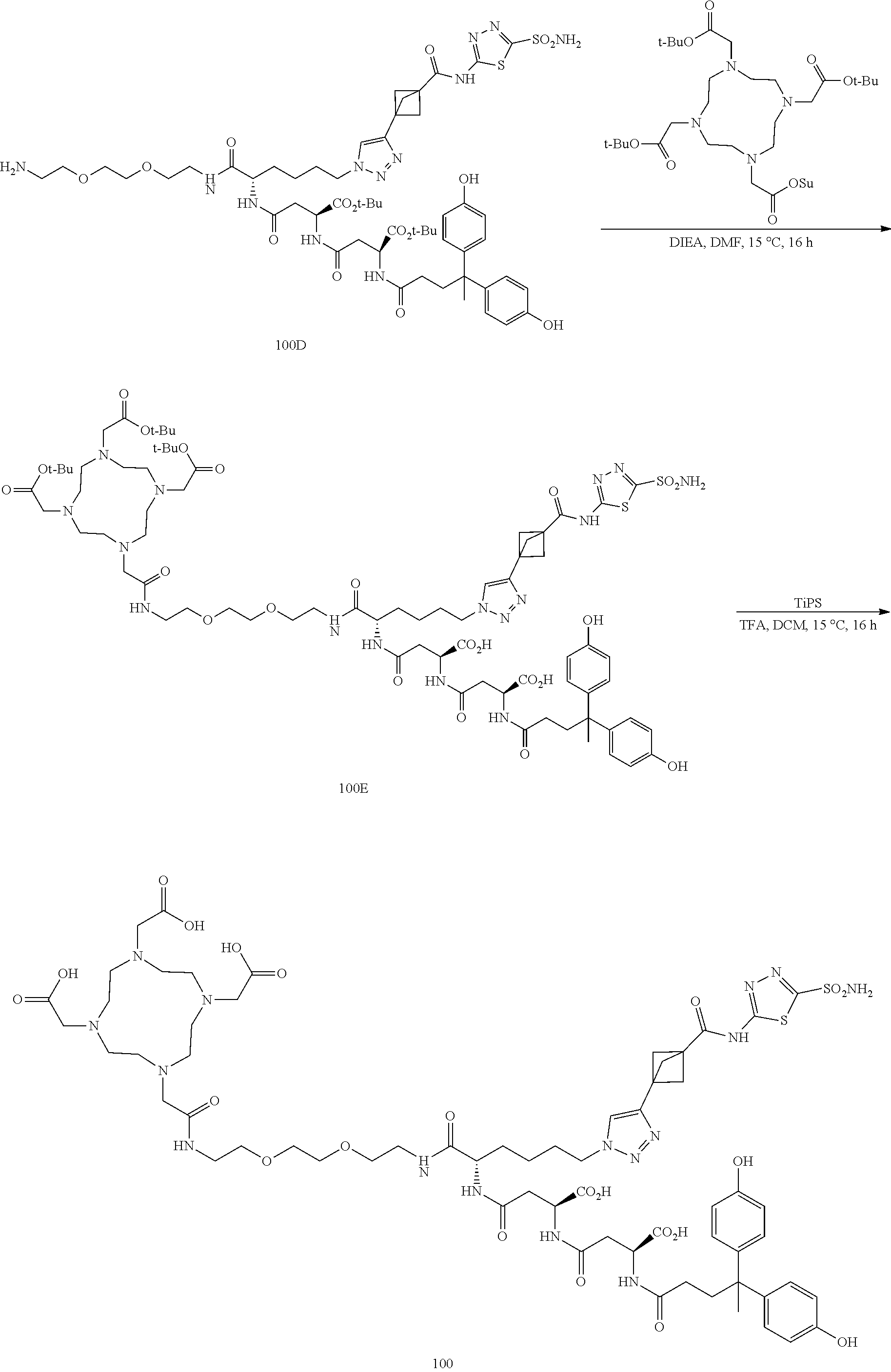

### 4.1. Synthesis of Compound 100B

To a solution of Intermediate 60 (551 mg, 493 μmol, 70% purity, 1.05 eq) in DMSO (5 mL) was added Intermediate 1 (200 mg, 469 μmol, 1 eq), then sodium ascorbate (93 mg, 469 μmol, 1 eq) and CuSO₄ (3.60 μL, 23.5 μmol, 0.05 eq) in H₂O (2.5 mL). The reaction mixture was stirred at 80° C. for 2 h, diluted with EtOAc (50 mL), extracted with H₂O (4×30 mL), combined organic layers washed with NH₄Cl (10 mL), dried (Na₂SO₄), filtered and concentrated under reduced pressure. The residue was purified by prep-HPLC (FA condition, column: Phenomenex Luna C18 75*30 mm*3 μm; mobile phase: [water (FA)-ACN]; B %: 30%-60%, 8 min) to give compound 100B (353 mg, 68% yield) as a white solid; LCMS [M+H]: 1081.2.

### 4.2. Synthesis of Compound 100C

To a mixture of compound 100B (323 mg, 299 μmol, 1 eq), T₃P (355 μL, 597 μmol, 2 eq) and DIEA (312 μL, 1.79 mmol, 6 eq) in DMF (1 mL) was added tert-butyl N-[2-[2-(2-aminoethoxy)ethoxy]ethyl]carbamate (111 mg, 448 μmol, 1.5 eq) in one portion at 15° C. under a nitrogen atmosphere. The mixture was stirred at 15° C. for 16 h, diluted with H₂O (50 mL), extracted with EtOAc (4×30 mL), the combined organic layers washed with brine (20 mL), dried (Na₂SO₄), filtered and concentrated under reduced pressure to give compound 100C (290 mg, 74% yield) as an oil; LCMS [M+H]: 1311.5.

### 4.3. Synthesis of Compound 100D

A mixture of compound 100C (270 mg, 206 μmol, 1 eq), TIPS (423 μL, 2.06 mmol, 10 eq) in DCM (2 mL) and TFA (2 mL) was degassed and purged with nitrogen 3 times, and the mixture stirred at 15° C. for 3 h under a nitrogen atmosphere. The mixture was concentrated under reduced pressure, the residue triturated with MTBE (3 mL) at 15° C. for 5 min and filtered to give crude compound 100D (242 mg, TFA salt) as a white solid; LCMS [M+H]: 1099.2.

### 4.4. Synthesis of Compound 100E

To a solution of compound 100D (210 mg, 173 μmol, 1 eq, TFA), tri-tert-butyl 2,2′,2″-(10-(2-((2,5-dioxopyrrolidin-1-yl)oxy)-2-oxoethyl)-1,4,7,10-tetraazacyclododecane-1,4,7-triyl)triacetate (116 mg, 173 μmol, 1 eq) in DMF (2 mL) was added DIEA (301 μL, 1.73 mmol, 10 eq). The mixture was stirred at 15° C. for 16 h and the mixture concentrated under reduced pressure. The residue was triturated with MTBE (3 mL) and filtered to give crude compound 100E (289 mg) as a white solid; LCMS [M+H]: 1653.8.

### 4.5. Synthesis of Compound 100

A mixture of compound 100E (289 mg, 175 μmol, 1 eq), triisopropylsilane (359 μL, 1.75 mmol, 10 eq) in DCM (3 mL) and TFA (3 mL) was degassed and purged with nitrogen 3 times, and the mixture stirred at 15° C. for 16 h under a nitrogen atmosphere. The mixture was concentrated under reduced pressure and purified by prep-HPLC (FA condition; column: Phenomenex Luna C18 200*40 mm*10 μm; mobile phase: [water (FA)-ACN]; B %: 5%-35%, 8 min) to give compound 100 (115 mg, 14% yield, FA salt); LCMS [M+H]: 1485.6; ¹H NMR (400 MHz, DMSO-d₆) δ 9.39-9.06 (m, 1H), 8.41-8.27 (m, 2H), 8.23-8.09 (m, 3H), 8.04-7.94 (m, 2H), 7.94-7.86 (m, 1H), 6.95-6.89 (m, 4H), 6.67-6.62 (m, 4H), 4.53-4.41 (m, 2H), 4.25 (brt, J=6.8 Hz, 2H), 4.21-4.13 (m, 1H), 3.50-3.39 (m, 18H), 3.27-3.11 (m, 13H), 3.03-2.94 (m, 10H), 2.67 (brd, J=1.6 Hz, 4H), 2.63-2.54 (m, 3H), 2.43 (s, 6H), 2.20-2.12 (m, 2H), 1.89-1.82 (m, 2H), 1.80-1.72 (m, 2H), 1.69-1.61 (m, 1H), 1.55-1.49 (m, 1H), 1.48-1.43 (m, 3H), 1.29-1.19 (m, 2H).

Compounds in table 9.1 were prepared by procedures depicted in Scheme D1.

TABLE 9.1

| Compound | m/z |
|---|---|
| 100.1 | [M + H]: 1513.3 |
| 100.2 | [M + H]: 1471.3 |
| 100.3 | [M + H]: 1487.4 |
| 100.4 | [M + H]: 1501.3 |
| 100.5 | [M + H]: 1499.3 |
| 100.6 | [M + H]: 1527.3 |
| 100.7 | [M + H]: 1513.4 |
| 100.8 | [M + H]: 1503.3 |
| 100.9 | [M + H]: 1489.3 |
| 100.10 | [M + H]: 1463.3 |
| 100.11 | [M + H]: 1437.3 |
| 100.12 | [M + H]: 1465.3 |
| 100.13 | [M + H]: 1451.3 |
| 100.14 | [M + H]: 1485.3 |
| 100.15 | [M + H]: 1541.4 |
| 100.16 | [M + H]: 1471.3 |
| 100.17 | [M/2 + H]: 750.3 |
| 100.18 | [M + H]: 1523.3 |
| 100.19 | [M/2 + H]: 1495.4 |

### D2. Synthesis of Compound 101

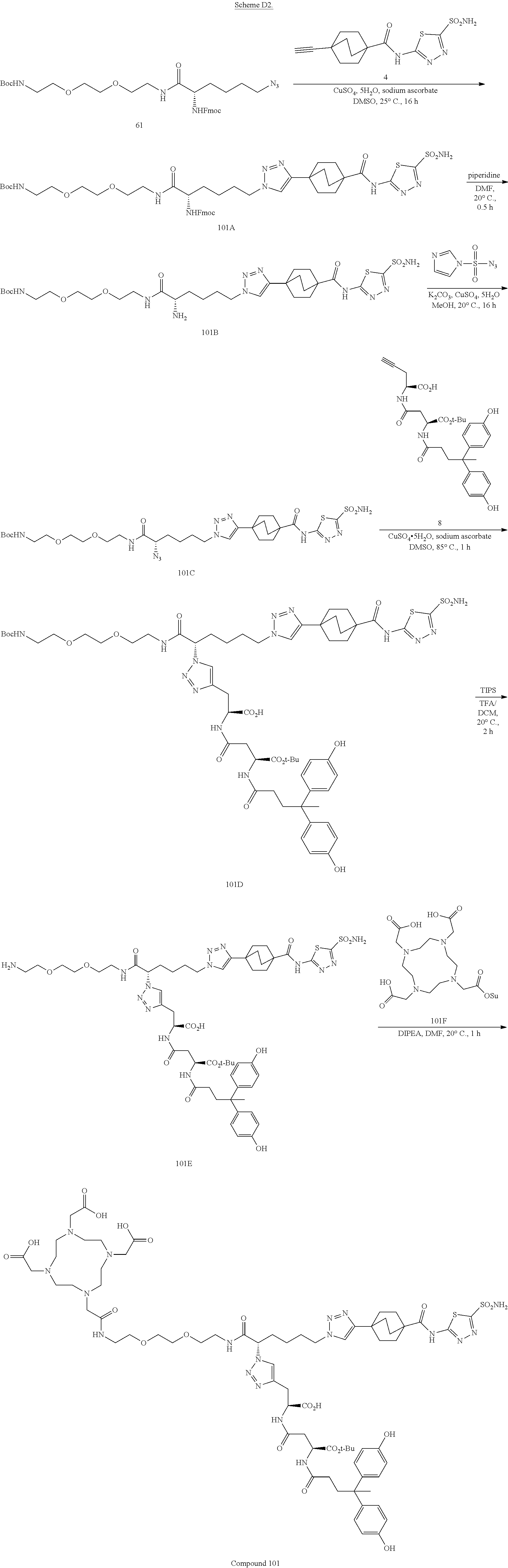

### 4.6 Preparation of Compound 101A

To a solution of Intermediate 61 (1.15 g, 1.84 mmol, 1 eq) and Intermediate 4 (689 mg, 2.02 mmol, 1.1 eq) in DMSO (10 mL) was added sodium ascorbate (365 mg, 1.84 mmol, 1 eq) and CuSO₄·5H₂O (46 mg, 184 μmol, 0.1 eq). The mixture was stirred at 25° C. for 16 h. The reaction mixture was added dropwise into H₂O (30 mL) and aq. NH₄Cl solution (10 mL), and the mixture stirred at 20° C. for 1 h. The mixture was filtered, and the filter cake dried to give compound 101A (1.4 g, 68% yield) as a white solid; LCMS [M+H]: 965.3.

### 4.7 Preparation of Compound 101B

To a solution of compound 101A (1.3 g, 1.35 mmol, 1 eq) in DMF (17 mL) was added piperidine (665 μL, 6.73 mmol, 5 eq). The mixture was stirred at 20° C. for 0.5 h and concentrated under reduced pressure. The crude product was triturated with MTBE (20 mL) to give compound 101B (820 mg, 71% yield, 87% purity) as a light green solid; LCMS [M+H]: 743.3; ¹H NMR (400 MHz, DMSO-d₆) δ 7.79 (br s, 2H), 7.44-7.09 (m, 1H), 6.77 (brs, 3H), 4.26 (br d, J=1.4 Hz, 2H), 3.50 (br s, 5H), 3.36 (br s, 6H), 3.07 (s, 4H), 1.80 (br s, 12H), 1.67-1.49 (m, 3H), 1.37 (s, 9H), 1.10 (s, 3H).

### 4.8 Preparation of Compound 101C

To a solution of compound 101B (820 mg, 1.10 mmol, 1 eq) in MeOH (8 mL) was added K₂CO₃ (229 mg, 1.66 mmol, 1.5 eq), CuSO₄·5H₂O (14 mg, 55.2 μmol, 0.05 eq) and 1H-imidazole-1-sulfonyl azide (229 mg, 1.32 mmol, 1.2 eq). The mixture was stirred at 20° C. for 16 h, filtered and concentrated under reduced pressure. The residue was purified by column chromatography (SiO₂, DCM:MeOH) and re-purified by prep-TLC (SiO₂, DCM:MeOH) to give compound 101C (250 mg, 29% yield) as a white solid; LCMS [M+H]: 769.3; ¹H NMR (400 MHz, DMSO-d₆) δ 13.08-12.39 (m, 1H), 8.30 (br s, 1H), 8.26 (br t, J=5.6 Hz, 1H), 7.84 (s, 1H), 6.77 (br t, J=5.6 Hz, 1H), 4.28 (t, J=7.0 Hz, 2H), 3.71 (t, J=7.0 Hz, 1H), 3.50 (s, 4H), 3.45-3.39 (m, 2H), 3.38-3.34 (m, 3H), 3.28-3.22 (m, 2H), 3.17 (d, J=4.6 Hz, 1H), 3.05 (q, J=6.0 Hz, 2H), 2.01-1.89 (m, 5H), 1.87-1.77 (m, 8H), 1.75-1.62 (m, 2H), 1.36 (s, 9H), 1.31-1.15 (m, 2H).

### 4.9 Preparation of Compound 101D

To a solution of compound 101C (176 mg, 228 μmol, 1 eq) and (S)-2-((S)-3-(4,4-bis(4-hydroxyphenyl)pentanamido)-4-(tert-butoxy)-4-oxobutanamido)pent-4-ynoic acid (277 mg, 502 μmol, 2.2 eq) in DMSO (4 mL) was added CuSO₄·5H₂O (5.70 mg, 22.8 μmol, 0.1 eq) and sodium ascorbate (45 mg, 228 μmol, 1 eq). The mixture was stirred at 85° C. for 1 h, the reaction mixture added dropwise into H₂O (15 mL) and aq. NH₄Cl solution (5 mL), and the mixture stirred at 20° C. for 1 h. The mixture was filtered and the filter cake dried to give compound 101D (220 mg, 55% yield, 75% purity) as a light brown solid; LCMS [M+H]: 1321.5; ¹H NMR (400 MHz, DMSO-d₆) δ 9.16 (br s, 2H), 8.52 (br s, 1H), 8.30-8.03 (m, 2H), 8.00-7.91 (m, 1H), 7.89-7.80 (m, 1H), 7.77-7.58 (m, 1H), 6.92 (br d, J=7.8 Hz, 4H), 6.75 (br s, 1H), 6.63 (brd, J=8.0 Hz, 4H), 5.37-5.12 (m, 1H), 4.38 (br s, 2H), 4.23 (br d, J=6.4 Hz, 2H), 3.48 (s, 5H), 3.44-3.32 (m, 11H), 3.23 (br s, 2H), 3.05 (br d, J=5.6 Hz, 3H), 2.97-2.79 (m, 1H), 2.42 (br d, J=4.4 Hz, 1H), 2.17 (br s, 2H), 1.94 (br s, 6H), 1.90-1.70 (m, 9H), 1.53-1.42 (m, 3H), 1.36-1.22 (m, 17H), 1.08 (br s, 2H).

### 4.10 Preparation of Compound 101E

To a solution of compound 101D (170 mg, 129 μmol, 1 eq) in DCM (1.5 mL) was added TIPS (528 μL, 2.57 mmol, 20 eq) and TFA (1.5 mL). The mixture was stirred at 20° C. for 2 h. The reaction mixture was concentrated under reduced pressure and the residue triturated with MTBE (15 mL) at 20° C. for 30 min. The mixture was filtered, and the filter cake dried to give crude compound 101E (220 mg) as a green solid; LCMS [M+H]: 1165.4; ¹H NMR (400 MHz, DMSO-d₆) δ 12.68 (br s, 1H), 9.76-8.89 (m, 1H), 8.50 (br t, J=5.2 Hz, 1H), 8.31 (s, 1H), 8.25 (br s, 1H), 8.02-7.86 (m, 2H), 7.83-7.69 (m, 3H), 6.91 (br d, J=8.6 Hz, 4H), 6.63 (br d, J=8.2 Hz, 4H), 5.23 (br t, J=7.6 Hz, 1H), 4.60-4.35 (m, 2H), 4.22 (br t, J=5.8 Hz, 2H), 3.62-3.49 (m, 5H), 3.46-3.36 (m, 2H), 3.23 (br d, J=5.6 Hz, 2H), 3.07 (s, 2H), 3.01-2.93 (m, 2H), 2.68-2.54 (m, 1H), 2.46-2.35 (m, 1H), 2.22-2.12 (m, 2H), 2.01-1.91 (m, 6H), 1.90-1.68 (m, 9H), 1.44 (s, 3H), 1.10 (s, 5H), 1.05-1.01 (m, 3H), 1.00-0.82 (m, 2H).

### 4.11 Preparation of Compound 101

To a solution of compound 101E (100 mg, 85.8 μmol, 1 eq) in DMF (2 mL) was added DIEA (75 μL, 429 μmol, 5 eq) and compound 101F (86 mg, 172 μmol, 2 eq) at 20° C., and the mixture stirred at 20° C. for 1 h. The reaction mixture was quenched by the addition of H₂O (0.1 mL) and concentrated under reduced pressure. The residue was purified by prep-HPLC (column: Phenomenex luna C18 100*40 mm*3 μm; mobile phase: [water (TFA)-ACN]; B %: 1%-40%, 8 min) and further purified by prep-HPLC (column: C18-1 150*30 mm*5 μm; mobile phase: [water (FA)-ACN]; B %: 5%-35%, 20 min) to give Compound 101 (5.2 mg, 3.9% yield); LCMS [M+H]: 1551.4; ¹H NMR (400 MHz, DMSO-d₆) δ 9.51-8.99 (m, 1H), 8.76-8.57 (m, 1H), 8.37-8.20 (m, 3H), 8.14 (br s, 1H), 7.98-7.85 (m, 2H), 7.78 (s, 1H), 6.90 (s, 4H), 6.63 (br d, J=8.2 Hz, 4H), 5.26 (br t, J=7.6 Hz, 1H), 4.47-4.38 (m, 2H), 4.21 (br d, J=5.8 Hz, 3H), 3.02 (br s, 30H), 2.71-2.54 (m, 8H), 2.47-2.39 (m, 2H), 2.23-2.10 (m, 3H), 2.08-1.69 (m, 21H), 1.44 (s, 4H), 1.18-0.98 (m, 3H).

Compounds in table 9.2 were prepared by procedures depicted in scheme D².

TABLE 9.2

| Compound | m/z |
|---|---|
| 101.1 | [M + H]: 1385.7 |
| 101.2 | [M + H]: 1509.8 |

### D3. Synthesis of Compound 102

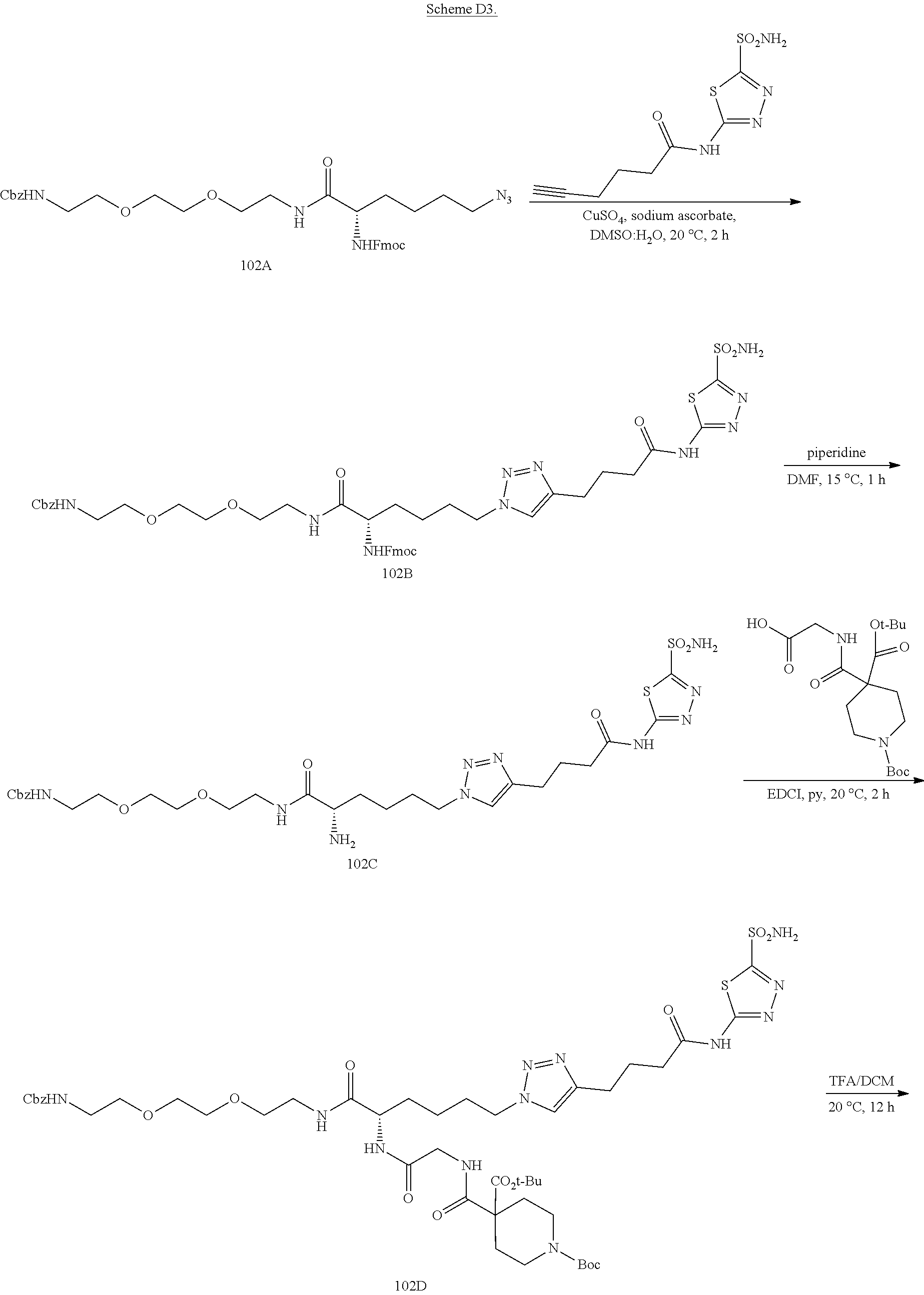

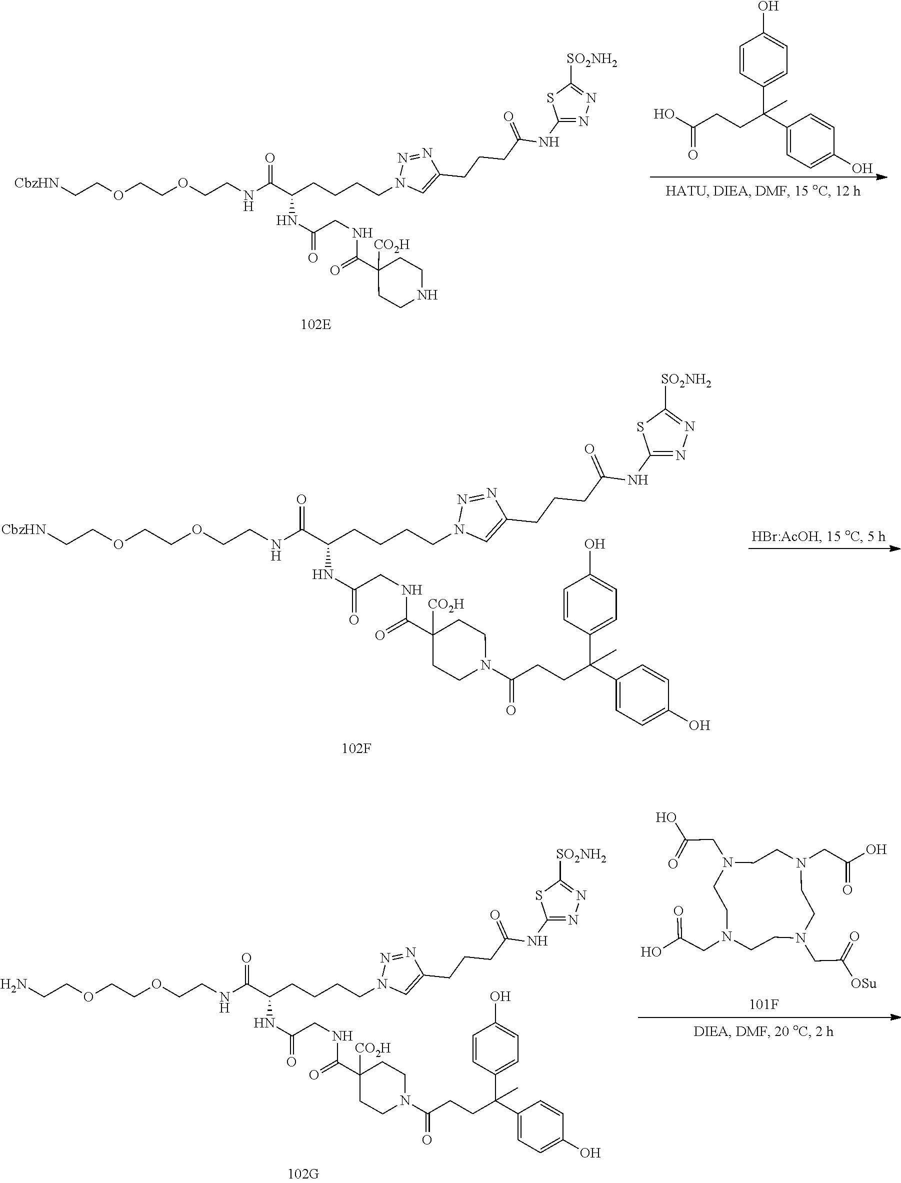

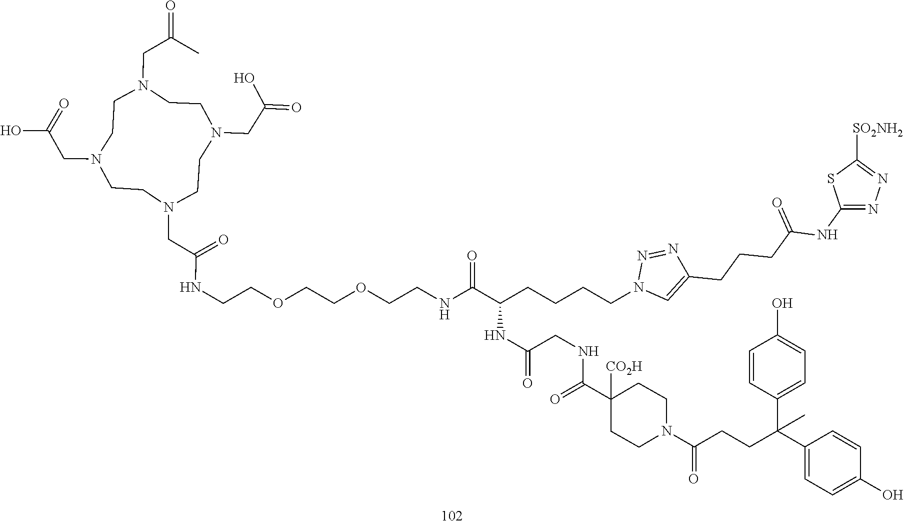 [SMILES: CC(=O)CN1CCN(CC(=O)O)CCN(CC(=O)NCCOCCOCCNC(=O)[C@H](CCCCn2cc(CCCC(=O)Nc3nnc(S(N)(=O)=O)s3)nn2)NC(=O)CNC(=O)C2(C(=O)O)CCN(C(=O)CCC(C)(c3ccc(O)cc3)c3ccc(O)cc3)CC2)CCN(CC(=O)O)CC1]

### 4.12 Preparation of Compound 102B

To a solution of compound 102A (13 g, 19.7 mmol, 1 eq) and N-(5-sulfamoyl-1,3,4-thiadiazol-2-yl)hex-5-ynamide (5.41 g, 19.7 mmol, 1 eq) in DMSO (30 mL) were added sodium ascorbate (3.91 g, 19.7 mmol, 1 eq) and CuSO₄ (303 μL, 1.97 mmol, 0.1 eq) in H₂O (10 mL) at 20° C. under a nitrogen atmosphere. The mixture was stirred at 20° C. for 2 h, poured into H₂O (80 mL), extracted with DCM (3×150 mL). The combined organic phase was dried (Na₂SO₄) and concentrated under reduced pressure. The residue was purified by chromatography column (SiO₂, DCM:MeOH) to give compound 102B (18.1 g, 98% yield) as a yellow solid; LCMS [M+H]: 933.2; ¹H NMR (400 MHz, CHLOROFORM-d) δ 7.73 (d, J=7.5 Hz, 2H), 7.55 (d, J=3.8 Hz, 2H), 7.41-7.28 (m, 8H), 7.16-6.90 (m, 2H), 6.21-6.12 (m, 1H), 6.06-5.94 (m, 1H), 5.59-5.46 (m, 1H), 5.17-4.97 (m, 2H), 4.43-4.09 (m, 6H), 3.62-3.27 (m, 12H), 2.83 (dd, J=4.1, 6.8 Hz, 1H), 2.20-2.07 (m, 2H), 1.88-1.70 (m, 5H), 1.59 (br s, 1H), 1.41-1.24 (m, 3H).

### 4.13 Preparation of Compound 102C

To a solution of compound 102B (8.0 g, 8.57 mmol, 1 eq) in DMF (10 mL) was added piperidine (847 μL, 8.57 mmol, 1 eq) at 20° C. under a nitrogen atmosphere. The mixture was stirred at 20° C. for 2 h and purified directly by chromatography column (SiO₂, EtOAc:MeOH) to give compound 102C (4.5 g, 74% yield) as a green solid; LCMS [M+H]: 711.2; ¹H NMR (400 MHz, DMSO-d₆) δ 7.85 (br s, 1H), 7.39-7.23 (m, 6H), 5.00 (s, 2H), 4.27 (br s, 2H), 3.53-3.08 (m, 21H), 2.65 (d, J=5.4 Hz, 2H), 2.01-1.67 (m, 6H), 1.12 (d, J=6.0 Hz, 2H).

### 4.14 Preparation of Compound 102D

To a solution of compound 102C (1.6 g, 4.14 mmol, 1 eq) and (1,4-bis(tert-butoxycarbonyl)piperidine-4-carbonyl)glycine (2.94 g, 4.14 mmol, 1 eq) in pyridine (10 mL) was added EDCl (1.59 g, 8.18 mmol, 2 eq) at 20° C. under a nitrogen atmosphere. The mixture was stirred at 20° C. for 2 h and concentrated under reduced pressure. The residue was purified by chromatography column (SiO₂, DCM:MeOH) to give compound 102D (4.0 g) as a yellow solid; LCMS [M+H]: 1079.5.

### 4.15 Preparation of Compound 102E

To a solution of compound 102D (3.7 g, 3.43 mmol, 1 eq) in DCM (5 mL) was added TFA (10 mL) and the reaction stirred at 20° C. for 12 h. The reaction mixture was concentrated under reduced pressure to give crude compound 102E (3.1 g, 98% yield) as a yellow oil that was used directly in the next reaction.

### 4.16 Preparation of Compound 102F

To a solution of compound 102E (1.0 g, 1.08 mmol, 1 eq) and 4,4-bis(4-hydroxyphenyl)pentanoic acid (310 mg, 1.08 mmol, 1 eq) in DMF (5 mL) were added DIEA (566 μL, 3.25 mmol, 3 eq) and HATU (412 mg, 1.08 mmol, 1 eq) at 20° C. under a nitrogen atmosphere. The reaction mixture was stirred for 12 h at 15° C., concentrated under reduced pressure and the residue purified by chromatography column (SiO₂, DCM:MeOH) to give compound 102F (1.8 g, 98% yield) as a yellow solid; LCMS [M+H]: 1191.6.

### 4.17 Preparation of Compound 102G

Compound 102F (1.4 g, 1.18 mmol, 1 eq) in HBr/AcOH (1.18 mmol, 10 mL, 20% purity, 1 eq) was stirred at 20° C. for 2 h. The reaction mixture was concentrated under reduced pressure and the residue triturated with MeCN (5 mL), filtered and the filter cake concentrated to give crude compound 102G (1.1 g, 71% yield) as a yellow solid that was used directly in the next reaction; LCMS [M+H]: 1057.3.

### 4.19 Preparation of Compound 102

To a solution of compound 102G (0.7 g, 483 μmol, 73% purity, 1 eq) in DMF (5 mL) was added DIEA (253 μL, 1.45 mmol, 3 eq) and 2,2′,2″-(10-(2-((2,5-dioxopyrrolidin-1-yl)oxy)-2-oxoethyl)-1,4,7,10-tetraazacyclododecane-1,4,7-triyl)triacetic acid (242 mg, 483 μmol, 1 eq) at 20° C. under a nitrogen atmosphere. The mixture was stirred at 20° C. for 2 h and the reaction mixture concentrated under reduced pressure. The residue was purified by prep-HPLC (column: Phenomenex luna C18 (250*70 mm, 15 μm); mobile phase: [water (TFA)-ACN]; B %: 10%-40%, 22 min) to give Compound 102 (0.15 g, 22% yield); LCMS [M+H]: 1443.4; ¹H NMR (400 MHz, DMSO-d₆) δ 13.07-12.96 (m, 1H), 9.23-9.15 (m, 1H), 8.50-8.37 (m, 1H), 8.35-8.28 (m, 2H), 8.08-7.94 (m, 2H), 7.90-7.76 (m, 2H), 6.99-6.88 (m, 4H), 6.72-6.59 (m, 4H), 4.28-4.17 (m, 3H), 4.03-3.66 (m, 8H), 3.63-3.56 (m, 4H), 3.50 (s, 5H), 3.43 (t, J=5.5 Hz, 4H), 3.37 (t, J=6.0 Hz, 4H), 3.29-3.23 (m, 4H), 3.18 (d, J=5.4 Hz, 9H), 3.09-3.04 (m, 3H), 2.70-2.55 (m, 5H), 2.20-2.13 (m, 2H), 2.04-1.91 (m, 6H), 1.89-1.73 (m, 4H), 1.52 (br s, 2H), 1.48 (s, 3H), 1.26-1.17 (m, 2H).

Compounds in table 9.3 were prepared by procedures depicted in scheme D³.

TABLE 9.3

| Compound | m/z |
|---|---|
| 102.1 | [M + H]: 1457.4 |
| 102.2 | [M + H]: 1428.5 |
| 102.3 | [M + 2H]: 1456.3 |
| 102.4 | [M + H]: 1429.3 |
| 102.5 | [M + H]: 1523.4 |
| 102.6 | [M + H]: 1481.3 |
| 102.7 | [M + H]: 1468.3 |
| 102.8 | [M + H]: 1417.7 |

### D4. Synthesis of Compound 103

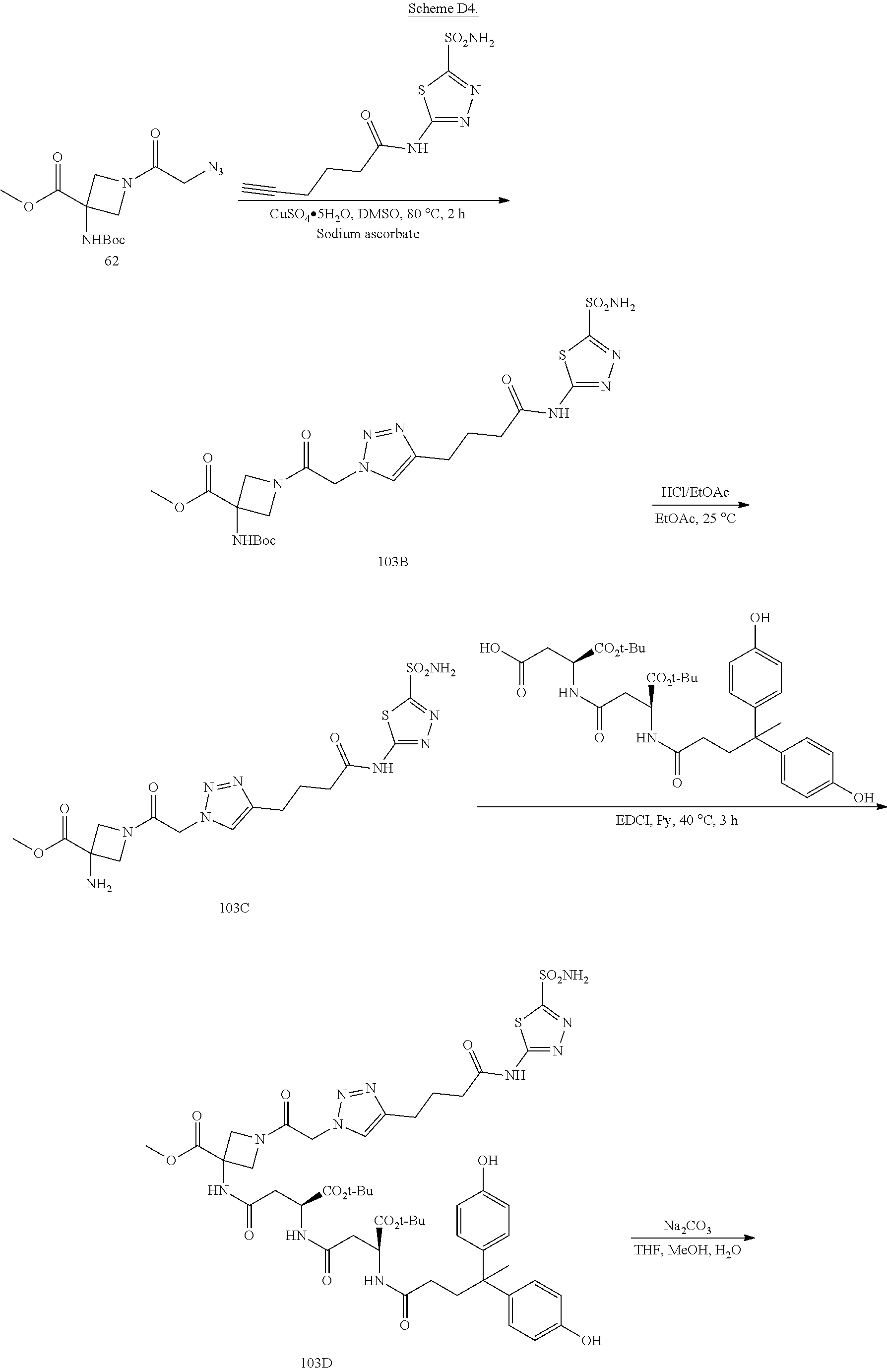

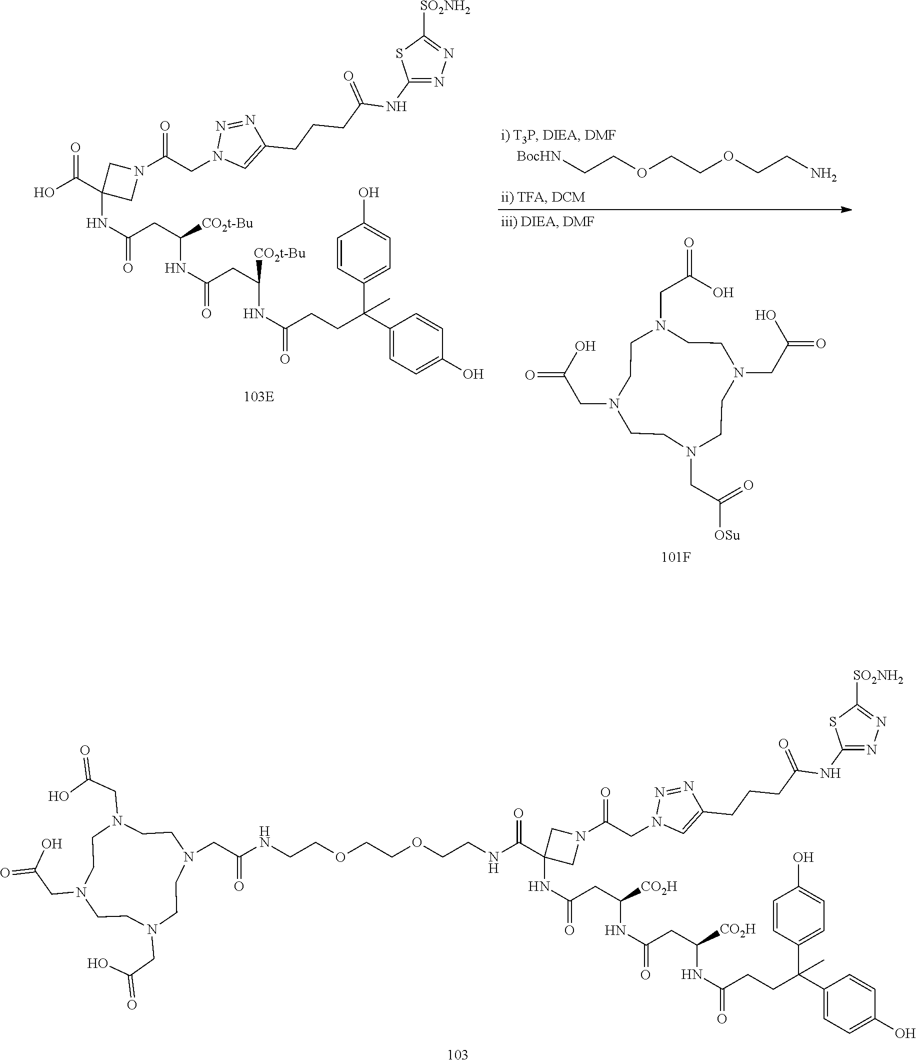

### 4.20 Preparation of Compound 103B

To a solution of compound 62 (1.0 g, 3.19 mmol, 1 eq) in DMSO (2 mL) was added sodium ascorbate (696 mg, 3.51 mmol, 1.1 eq), N-(5-sulfamoyl-1,3,4-thiadiazol-2-yl)hex-5-ynamide (1.01 g, 3.67 mmol, 1.15 eq) and CuSO₄·5H₂O (48 mg, 192 μmol, 0.06 eq). The mixture was stirred at 80° C. for 2 h, extracted with EtOAc (2×200 mL), combined organics washed with brine (150 mL), dried (Na₂SO₄), filtered and concentrated under reduced pressure to give compound 103B (1.4 g, 75% yield) as a white solid; LCMS [M+H]: 588.1.

### 4.21 Preparation of Compound 103C

To a solution of Compound 103B (1.3 g, 2.21 mmol, 1 eq) in EtOAc (10 mL) was added 4 M HCl in EtOAc (10 mL). The mixture was stirred at 25° C. for 1 h, the reaction mixture filtered and concentrated under reduced pressure to give compound 103C (1 g, 93% yield) as a white solid; LCMS [M+H]: 488.0.

### 4.22 Preparation of Compound 103D

To a solution of compound 103C (1.0 g, 2.05 mmol, 1 eq) in pyridine (2 mL) was added EDCl (786 mg, 4.10 mmol, 2 eq) and compound 30 (645 mg, 1.03 mmol, 0.5 eq). The mixture was stirred at 40° C. for 3 h, filtered and concentrated under reduced pressure. The residue was purified by column chromatography (SiO₂, MeOH:DCM) to give compound 103D (800 mg, 18% yield) as a white solid; LCMS [M+H]: 1098.4.

### 4.23 Preparation of Compound 103E

To a solution of compound 103D (700 mg, 637 μmol, 1 eq) in THF (9 mL), H₂O (1 mL) and MeOH (1 mL) was added Na₂CO₃ (270 mg, 2.55 mmol, 4 eq). The mixture was stirred at 25° C. for 1 h, the reaction mixture adjusted to pH=7 with 1M HCl, filtered and concentrated under reduced pressure to give compound 103E (500 mg, 72% yield) as a white solid; LCMS [M+H]: 1084.5.

### 4.47 Preparation of Compound 103

Compound 103 was prepared as per reactions previously depicted; LCMS [M+H]: 1488.3.

Compounds in table 9.4 were prepared by procedures depicted in scheme D4.

TABLE 9.4

| Compound | m/z |
|---|---|
| 103.1 | [M + H]: 1502.3 |
| 103.2 | [M + H]: 1530.2 |
| 103.3 | [M + H]: 1474.3 |
| 103.4 | [M + H]: 1502.3 |

### D5. Synthesis of Compound 104

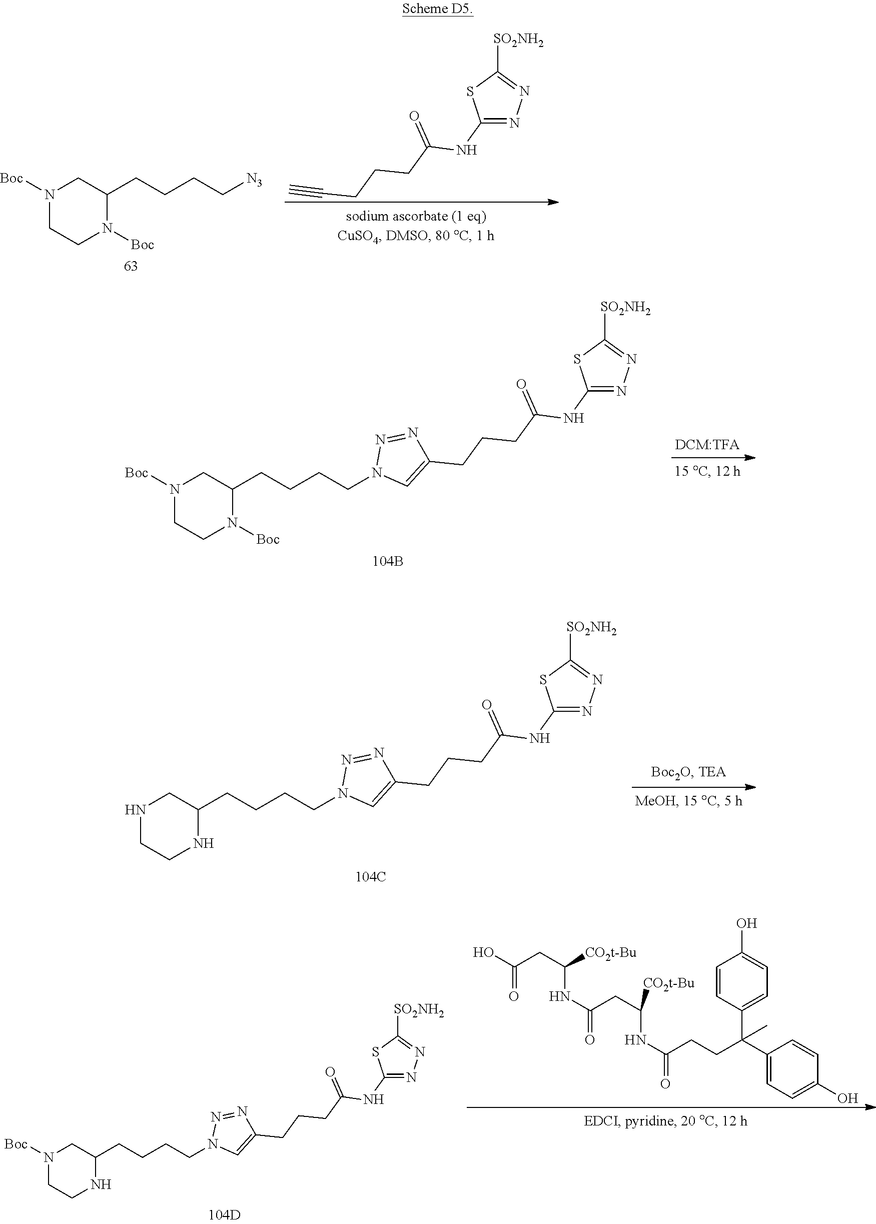

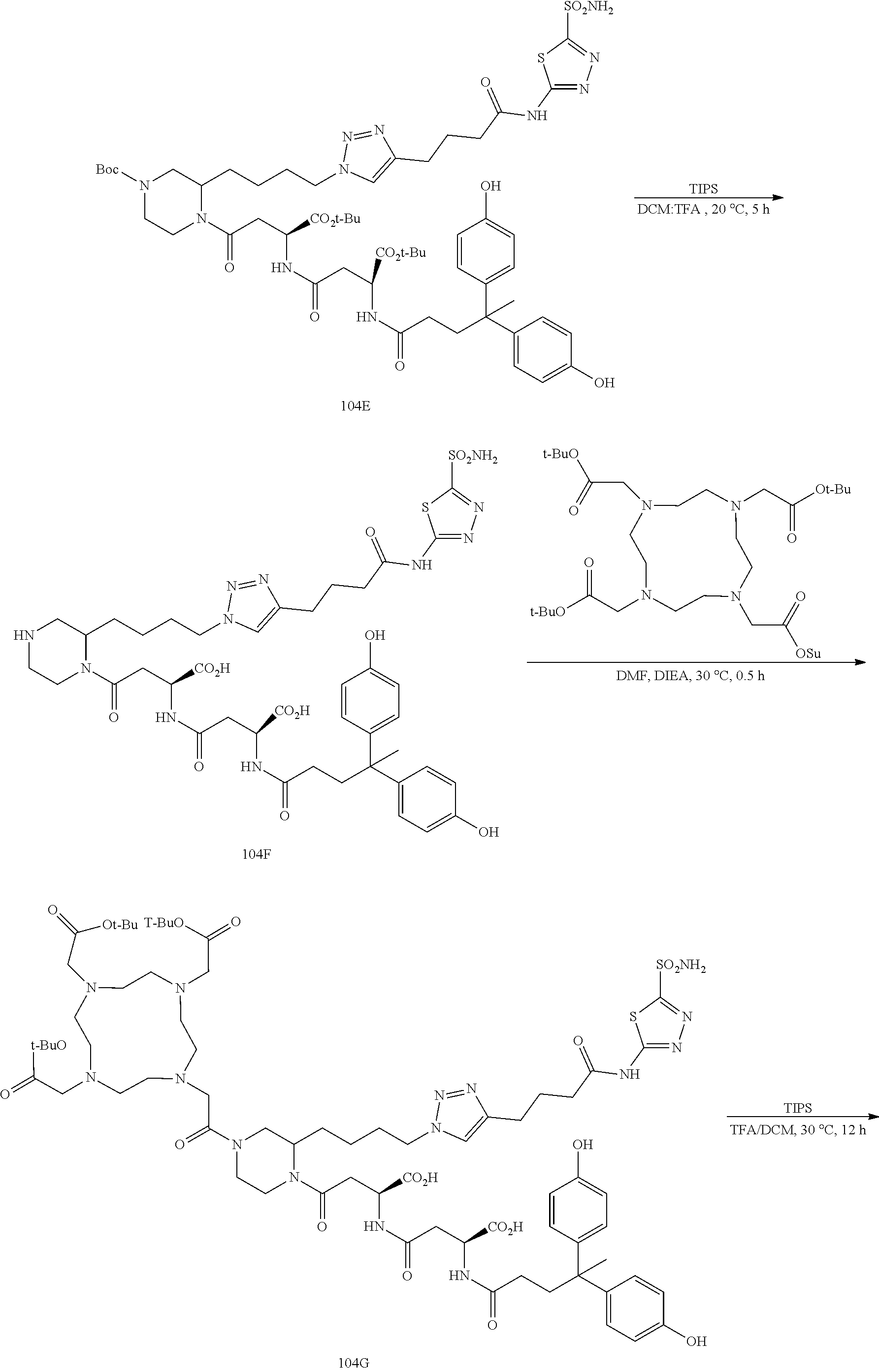

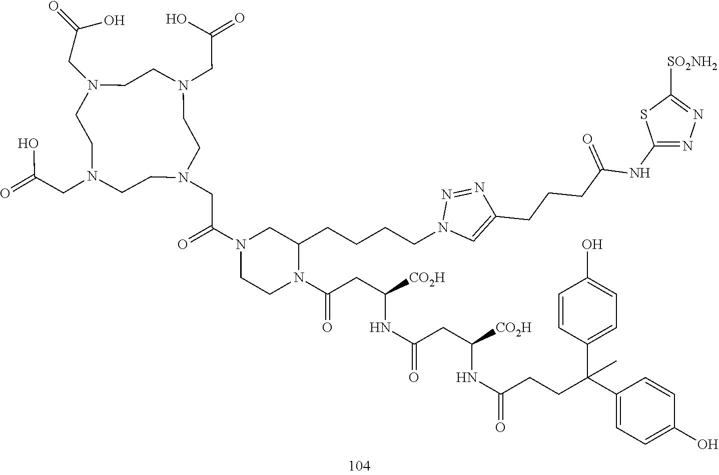 [SMILES: CC(CCC(=O)N[C@@H](CC(=O)N[C@@H](CC(=O)N1CCN(C(=O)CN2CCN(CC(=O)O)CCN(CC(=O)O)CCN(CC(=O)O)CC2)CC1CCCCn1cc(CCCC(=O)Nc2nnc(S(N)(=O)=O)s2)nn1)C(=O)O)C(=O)O)(c1ccc(O)cc1)c1ccc(O)cc1]

### 4.24 Synthesis of Compound 9

To a solution of compound 63 (10.0 g, 26.1 mmol, 1 eq) and N-(5-sulfamoyl-1,3,4-thiadiazol-2-yl)hex-5-ynamide (5.72 g, 20.9 mmol, 0.8 eq) in DMSO (80 mL) was added sodium ascorbate (5.17 g, 26.1 mmol, 1 eq) and CuSO₄·5H₂O (325.55 mg, 1.30 mmol, 0.05 eq) at 15° C., and the mixture stirred at 80° C. for 1 h. The reaction mixture was poured into H₂O (160 mL) and aq. NH₄Cl solution (10 mL), filtered and the filter cake concentrated under reduced pressure to give compound 104B (17.2 g, 99% yield) as a blue oil; LCMS [M+H]: 658.2; ¹H NMR (400 MHz, DMSO-d₆) δ 8.25 (br s, 2H), 7.82 (s, 1H), 4.24 (t, J=6.6 Hz, 2H), 4.01-3.60 (m, 4H), 2.93-2.71 (m, 2H), 2.68-2.53 (m, 4H), 1.90 (m, 2H), 1.84-1.67 (m, 2H), 1.56-1.39 (m, 2H), 1.37-1.29 (m, 18H), 1.22-1.00 (m, 2H).

### 4.25 Synthesis of Compound 104C

A mixture of compound 104B (17.2 g, 26.1 mmol, 1 eq) in DCM (15 mL) and TFA (15 mL) was stirred at 15° C. for 12 h. The reaction mixture was concentrated under reduced pressure to give compound 104C (19.5 g, 73% yield, TFA salt) as a yellow solid; LCMS [M+H]: 458.1; ¹H NMR (400 MHz, DMSO-d₆) δ 7.88 (br s, 1H), 4.29 (t, J=6.5 Hz, 2H), 3.33 (t, J=6.7 Hz, 1H), 2.90-2.52 (m, 10H), 2.05-1.86 (m, 2H), 1.78 (br s, 2H), 1.38 (br s, 2H), 1.31-1.17 (m, 2H).

### 4.26 Synthesis of Compound 104D

To a solution of compound 104C (13.0 g, 28.3 mmol, 1 eq) and TEA (19.7 mL, 142 mmol, 5 eq) in MeOH (100 mL) was added Boc₂O (6.5 mL, 28.3 mmol, 1 eq) in MeOH (50 mL) at 15° C., and the mixture stirred at 15° C. for 5 h. The reaction mixture was concentrated under reduced pressure and the residue purified by column chromatography (SiO₂, EtOAc:MeOH) to give compound 104D (12.5 g, 79% yield) as a red oil; LCMS [M+H]: 558.2; ¹H NMR (400 MHz, DMSO-d₆) δ 7.88 (s, 1H), 4.30 (t, J=7.0 Hz, 2H), 3.83 (d, J=13.2 Hz, 3H), 3.01-2.91 (m, 2H), 2.91-2.77 (m, 2H), 2.66 (t, J=7.5 Hz, 2H), 2.59 (t, J=7.3 Hz, 2H), 1.98-1.90 (m, 2H), 1.81 (q, 2H), 1.53-1.45 (m, 2H), 1.39 (s, 9H), 1.34-1.25 (m, 2H).

### 4.27 Synthesis of Compound 104E

To a solution of compound 104D (5.4 g, 9.7 mmol, 1 eq) and Intermediate 30 (6.09 g, 9.68 mmol, 1 eq) in pyridine (20 mL) was added EDCl (3.71 g, 19.4 mmol, 2 eq) at 15° C., and the mixture stirred at 20° C. for 12 h. The reaction mixture was concentrated under reduced pressure, the residue purified by column chromatography (SiO₂, EtOAc:MeOH) and prep-HPLC (column: Phenomenex luna C18 (250*70 mm, 15 μm); mobile phase: [water (FA)-ACN]; B %: 40%-70%, 20 min) to give compound 104E (700 mg) as a white solid; LCMS [M+H]: 1168.6; ¹H NMR (400 MHz, DMSO-d₆) δ 13.27-12.60 (m, 1H), 9.15 (s, 2H), 8.28 (br s, 2H), 8.13 (d, J=6.8 Hz, 1H), 8.05 (d, J=8.0 Hz, 1H), 7.96-7.88 (m, 1H), 7.85 (s, 1H), 6.92 (d, J=8.3 Hz, 4H), 6.64 (d, J=8.5 Hz, 4H), 4.50 (br d, J=5.1 Hz, 1H), 4.44-4.32 (m, 2H), 4.32-4.18 (m, 2H), 3.94-3.72 (m, 2H), 3.58 (dd, J=2.1, 11.1 Hz, 1H), 3.08 (t, J=11.7 Hz, 1H), 2.74-2.53 (m, 8H), 2.21-2.11 (m, 2H), 1.99-1.88 (m, 2H), 1.88-1.71 (m, 4H), 1.54-1.41 (m, 5H), 1.41-1.29 (m, 28H), 1.26-1.08 (m, 2H).

### 4.28 Synthesis of Compound 104F

To a solution of compound 104E (200 mg, 171 μmol, 1 eq) in DCM (1 mL) was added TIPS (352 μL, 1.71 mmol, 10 eq) and TFA (1 mL), and the mixture stirred at 15° C. for 12 h. The reaction mixture was concentrated under reduced pressure and the residue triturated with MTBE at 20° C. for 0.5 h to give crude compound 104F (200 mg, TFA) as yellow solid; LCMS [M+H]: 956.2.

### 4.29 Synthesis of Compound 104G

To a solution of compound 104F (210 mg, 220 μmol, 1 eq) and DIEA (383 μL, 2.20 mmol, 10 eq) in DMF (3 mL) was added tri-tert-butyl 2,2′,2″-(10-(2-((2,5-dioxopyrrolidin-1-yl)oxy)-2-oxoethyl)-1,4,7,10-tetraazacyclododecane-1,4,7-triyl)triacetate (221 mg, 329 μmol, 1.5 eq), and the mixture stirred at 30° C. for 0.5 h. The reaction mixture was concentrated under reduced pressure and the residue triturated with ACN at 20° C. to give compound 104G (230 mg, 69% yield) as a yellow solid; LCMS [M+H]: 1511.2.

### 4.30 Synthesis of Compound 104

To a solution of compound 104G (230 mg, 152 μmol, 1 eq) in DCM (1 mL) was added TIPS (313 μL, 1.52 mmol, 10 eq) and TFA (2 mL), and the mixture stirred at 30° C. for 12 h. The reaction mixture was concentrated under reduced pressure and the residue purified by prep-HPLC (column: Phenomenex Luna C18 150*30 mm*5 μm; mobile phase: [water (TFA)-ACN]; B %: 5%-25%, 8 min) to give Compound 104 (90 mg, 43% yield); LCMS [M+H]: 1342.3; ¹H NMR (400 MHz, DMSO-d₆) δ 13.00 (br s, 1H), 12.84-12.39 (m, 2H), 9.30-9.04 (m, 1H), 8.32 (s, 2H), 8.23-8.04 (m, 1H), 7.92 (t, J=7.7 Hz, 1H), 7.81 (dd, J=2.8, 7.3 Hz, 1H), 6.93 (d, J=8.5 Hz, 4H), 6.64 (d, J=8.6 Hz, 4H), 4.68-4.55 (m, 1H), 4.53-4.43 (m, 2H), 4.33-4.09 (m, 6H), 4.07-3.86 (m, 4H), 3.80-3.50 (m, 8H), 3.47-3.22 (m, 10H), 3.20-2.98 (m, 9H), 2.77-2.55 (m, 8H), 2.22-2.12 (m, 2H), 1.94 (q, 2H), 1.88-1.70 (m, 4H), 1.45 (s, 3H), 1.29-1.09 (m, 2H).

Compounds in table 9.5 were prepared by procedures depicted in scheme D5.

TABLE 9.5

| Compound | m/z |
|---|---|
| 104.1 | [M + H]: 1394.6 |
| 104.2 | [M + H]: 1394.3 |

### D6. Synthesis of Compound 106

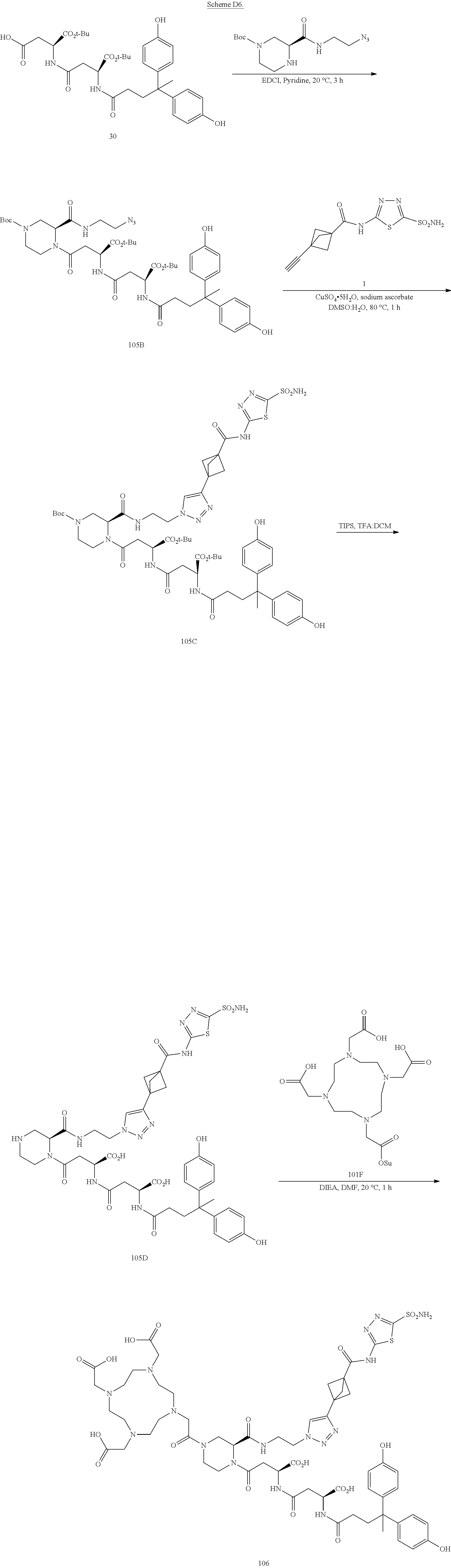

### 4.31 Synthesis of Compound 106B

To a solution of tert-butyl(S)-3-((2-azidoethyl) carbamoyl) piperazine-1-carboxylate (300 mg, 1.01 mmol, 1 eq) and compound 30 (316 mg, 503 μmol, 0.5 eq) in pyridine (5 mL) was added EDCl (231 mg, 1.21 mmol, 1.2 eq) at 25° C. The mixture was stirred at 20° C. for 3 h. The reaction was extracted with EtOAc (3×40 mL), the combined organic layers washed with brine (3×10 mL), dried (Na₂SO₄), filtered and concentrated under reduced pressure. The residue was purified by column chromatography (SiO₂, petroleum ether:EtOAc) to give compound 106B (0.59 g, 65% yield) as a white solid; LCMS [M+H]: 909.5.

### 4.32 Synthesis of Compound 106C

To a solution of compound 106B (500 mg, 550 μmol, 1 eq) and compound 1 (164 mg, 550 μmol, 1 eq) in DMSO (5 mL) and H₂O (1 mL) was added CuSO₄·5H₂O (6.87 mg, 27.5 μmol, 0.05 eq) and sodium ascorbate (120 mg, 605 μmol, 1.1 eq) at 20° C. The mixture was stirred at 80° C. for 1 h. The reaction mixture was concentrated under reduced pressure and recrystallization from H₂O (15 mL) to give compound 106C (590 mg, 89% yield) as a green solid; LCMS [M+H]: 1207.5.

### 4.33 Synthesis of Compound 106D

To a solution of compound 106C (0.5 g, 414 μmol, 1 eq) in DCM (5 mL) and TFA (5 mL) was added TIPS (851 μL, 4.14 mmol, 10 eq) at 20° C. The reaction mixture was stirred at 20° C. for 2 h, concentrated under reduced pressure and the residue triturated with MTBE at 20° C. for 10 min to give compound 106D (414 μmol, 99% yield, TFA salt) as a green solid; LCMS [M+H]: 995.3.

### 4.34 Synthesis of Compound 106

To a solution of compound 106D (0.45 g, 452 μmol, 1 eq) in DMF (5 mL) was added DIEA (788 μL, 4.52 mmol, 10 eq) and 2,2′,2″-(10-(2-((2,5-dioxopyrrolidin-1-yl)oxy)-2-oxoethyl)-1,4,7,10-tetraazacyclododecane-1,4,7-triyl)triacetic acid (272 mg, 543 μmol, 1.2 eq) at 20° C. The mixture was stirred at 20° C. for 1 h. The reaction mixture was concentrated under reduced pressure and the residue purified by prep-HPLC (PhenomenexC18 80*30 mm*3um mobile phase: [water (TFA)-ACN]; B %: 5%-35%, 8 min.) to give Compound 106 (130 mg, 21% yield); LCMS [M+H]: 1381.5; ¹H NMR (400 MHz, DMSO-d₆) δ 13.33-12.27 (m, 3H), 9.28-8.98 (m, 1H), 8.40-8.24 (m, 2H), 8.23-7.98 (m, 1H), 7.98-7.74 (m, 2H), 6.92 (br d, J=8.3 Hz, 4H), 6.64 (d, J=8.7 Hz, 4H), 5.00-4.72 (m, 1H), 4.68-3.95 (m, 11H), 3.85-3.50 (m, 9H), 3.20-2.78 (m, 15H), 2.71-2.55 (m, 5H), 2.44 (s, 7H), 2.19-2.03 (m, 1H), 2.19-1.82 (m, 2H), 1.45 (s, 3H).

### D7. Synthesis of Compound 107

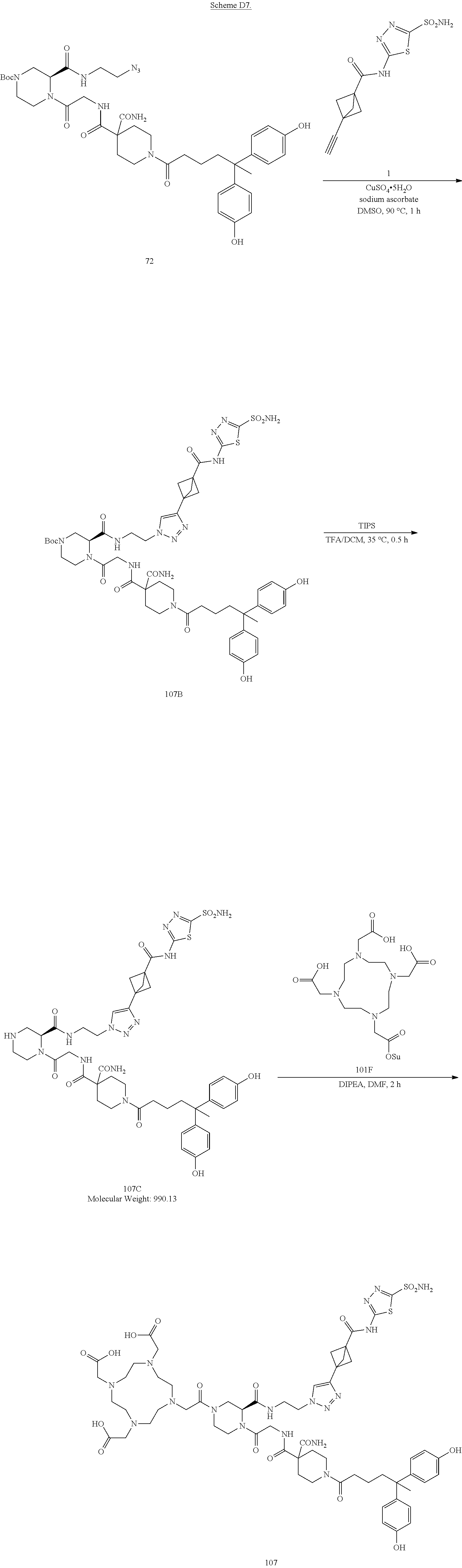

### 4.35 Synthesis of Compound 107B

To a solution of Intermediate 72 (11.5 g, 13.1 mmol, 1 eq) and compound 1 (3.51 g, 11.8 mmol, 0.9 eq) in DMSO (57 mL) was added CuSO₄·5H₂O (653 mg, 2.61 mmol, 0.2 eq) and sodium ascorbate (5.18 g, 26.1 mmol, 2 eq). The mixture was stirred at 90° C. for 1 h. The reaction was repeated and combined. Silica gel (26 g) was added to the reaction mixture was stirred at 40° C. for 0.5 h. The mixture was filtered and the filtrate was added dropwise to H₂O (230 mL) and NH₄Cl (60 mL) at 20° C. over 10 min. The mixture was filtered, the filter cake washed with H₂O (300 mL) and the filter cake concentrated under reduced pressure. The residue was dissolved in DCM (130 mL) and MeOH (130 mL). Silica gel (26 g) was added into the mixture and stirred at 40° C. for 0.5 h. The mixture was filtered and the filtrate concentrated under reduced pressure. The residue was purified by column chromatography (SiO₂, DCM:MeOH) to give compound 107B (23 g, 80% yield) as a white solid; LCMS [M+H]: 1090.6; ¹H NMR (400 MHz, DMSO-d₆) δ 13.19 (brs, 1H), 9.11 (s, 2H), 8.32 (br s, 3H), 7.93 (br s, 2H), 7.50-7.39 (m, 1H), 7.25 (brs, 1H), 6.93 (d, J=8.6 Hz, 4H), 6.62 (d, J=8.8 Hz, 4H), 4.70 (br s, 1H), 4.51-4.00 (m, 5H), 3.98-3.84 (m, 1H), 3.80-3.39 (m, 7H), 3.24-3.04 (m, 4H), 2.70-2.63 (m, 1H), 2.46-2.35 (m, 7H), 2.24 (brt, J=6.8 Hz, 2H), 2.09-1.73 (m, 7H), 1.47 (s, 3H), 1.38 (s, 9H), 1.23 (br s, 3H).

### 4.36 Synthesis of Compound 107C

To a solution of compound 107B (11.5 g, 10.6 mmol, 1 eq) in DCM (60 mL) was added triisopropylsilane (43.3 mL, 211 mmol, 20 eq) and TFA (60 mL) and the reaction mixture stirred at 35° C. for 0.5 h. The reaction was repeated and combined. The mixture was concentrated under reduced pressure, the residue triturated with MTBE (100 mL) at 20° C. for 20 min twice, the mixture filtered and the filter cake concentrated under reduced pressure to give compound 107C (27 g, 93% yield, 3 TFA) as a white solid; LCMS [M+H]: 990.6; ¹H NMR (400 MHz, DMSO-d₆) δ 13.20 (s, 1H), 9.49-9.26 (m, 1H), 8.72-8.51 (m, 1H), 8.35 (s, 2H), 8.22 (br s, 1H), 8.02 (s, 1H), 7.95-7.83 (m, 1H), 7.38 (br s, 1H), 7.34-7.26 (m, 1H), 7.18-6.99 (m, 1H), 6.93 (d, J=8.6 Hz, 4H), 6.62 (d, J=8.6 Hz, 4H), 5.15-4.82 (m, 1H), 4.53-4.33 (m, 2H), 4.31-4.09 (m, 1H), 4.04-3.80 (m, 2H), 3.67 (br d, J=11.0 Hz, 1H), 3.64-3.48 (m, 3H), 3.45-3.15 (m, 5H), 3.13-2.95 (m, 1H), 2.85 (br d, J=9.0 Hz, 1H), 2.47-2.40 (m, 6H), 2.25 (br t, J=7.2 Hz, 2H), 2.07-1.78 (m, 6H), 1.47 (s, 3H), 1.29-1.16 (m, 2H).

### 4.37 Synthesis of Compound 107

2,2′,2″-(10-(2-((2,5-Dioxopyrrolidin-1-yl)oxy)-2-oxoethyl)-1,4,7,10-tetraazacyclododecane-1,4,7-triyl)triacetic acid (897 mg, 1.18 mmol, 1.3 eq) was added to a solution of compound 107C (1.0 g, 0.91 mmol, 1 eq), DIEA (790 μL, 4.54 mmol, 5 eq), and DMF (10 mL) at 0° C. The mixture stirred at 0° C. for 10 minutes and stirred at room temperature for 2 h. The reaction was purified directly by prep-HPLC (5-45% CH₃CN in water with 0.5% AcOH) to give Compound 107 (642 mg, 51%); LCMS [M/2+H]: 688.9; ¹H NMR (400 MHz, DMSO) δ 9.22 (s, 2H), 8.48 (s, 1H), 8.27 (m, 2H), 8.06 (s, 1H), 7.90 (m, 1H), 7.47 (m, 1H), 7.33-7.23 (m, 1H), 6.92 (d, J=8.4 Hz, 4H), 6.62 (d, J=8.3 Hz, 4H), 4.72 (m, 1H), 4.42 (d, J=6.2 Hz, 2H), 4.20 (m, 3H), 4.02-3.63 (m, 6H), 3.51 (m, 13H), 3.21 (m, 4H), 2.78 (m, 13H), 2.45-2.37 (m, 6H), 2.24 (m, 2H), 1.94 (s, 3H), 1.81 (m, 2H), 1.47 (s, 3H), 1.22 (t, J=8.2 Hz, 2H).

Compounds in table 9.6 were prepared by procedures depicted in scheme D7.

TABLE 9.6

| Compound | m/z |
|---|---|
| 107.1 | [M + H]: 1409.2 |
| 107.2 | [M + H]: 1410.3 |
| 107.3 | [M + H]: 1381.2 |
| 107.4 | [M + H]: 1363.3 |
| 107.5 | [M + H]: 1395.6 |
| 107.6 | [M + H]: 1376.5 |
| 107.7 | [M + H]: 1299.3 |
| 107.8 | [M + H]: 1379.3 |
| 107.9 | [M + H]: 1419.3 |
| 107.10 | [M + H]: 1275.3 |
| 107.11 | [M + H]: 1280.2 |
| 107.12 | [M + H]: 1377.3 |
| 107.13 | [M + H]: 1395.4 |
| 107.14 | [M + H]: 1338.2 |
| 107.15 | [M + H]: 1509.8 |
| 107.16 | [M + H]: 1170.5 |
| 107.17 | [M + H]: 1156.3 |
| 107.18 | [M + H]: 1154.3 |
| 107.19 | [M + H]: 1378.7 |
| 107.20 | [M + H]: 1421.8 |
| 107.21 | [M + H]: 1381.3 |
| 107.22 | [M + H]: 1391.4 |
| 107.23 | [M + H]: 1421.5 |
| 107.24 | [M + H]: 1391.5 |
| 107.25 | [M + H]: 1406.6 |
| 107.26 | [M + H]: 1351.5 |
| 107.27 | [M + H]: 1377.5 |
| 107.28 | [M + H]: 1387.4 |
| 107.29 | [M + H]: 1393.6 |
| 107.30 | [M + H]: 1425.4 |
| 107.31 | [M + H]: 1358.5 |
| 107.32 | [M + H]: 1421.6 |
| 107.33 | [M + H]: 1417.6 |
| 107.34 | [M + H]: 1390.5 |
| 107.35 | [M + H]: 1405.3 |
| 107.36 | [M + H]: 1425.3 |
| 107.37 | [M/2 + H]: 680.8 |
| 107.38 | [M + H]: 1380.6 |
| 107.39 | [M + H]: 1395.3 |
| 107.40 | [M + H]: 1405.4 |
| 107.41 | [M + H]: 1417.7 |
| 107.42 | [M + H]: 1443.8 |
| 107.43 | [M + H]: 1423.7 |
| 107.44 | [M + H]: 1392.5 |
| 107.45 | [M/2 + H]: 1469.7 |
| 107.46 | [M + H]: 1437.8 |
| 107.47 | [M + H]: 1379.6 |
| 107.48 | [M + H]: 1389.7 |
| 107.49 | [M + H]: 1346.6 |
| 107.50 | [M + H]: 1380.6 |
| 107.51 | [M + H]: 1362.7 |
| 107.52 | [M + H]: 1374.7 |
| 107.53 | [M + H]: 1372.8 |
| 107.54 | [M + H]: 1403.3 |
| 107.55 | [M + H]: 1396.4 |
| 107.56 | [M + H]: 1377.3 |
| 107.57 | [M + H]: 1378.9 |
| 107.58 | [M/2 + H]: 710.8 |
| 107.59 | [M/2 + H]: 711.2 |
| 107.60 | [M/2 + H]: 713.7 |
| 107.61 | [M/2 + H]: 713.7 |

### D8. Synthesis of Compound 108

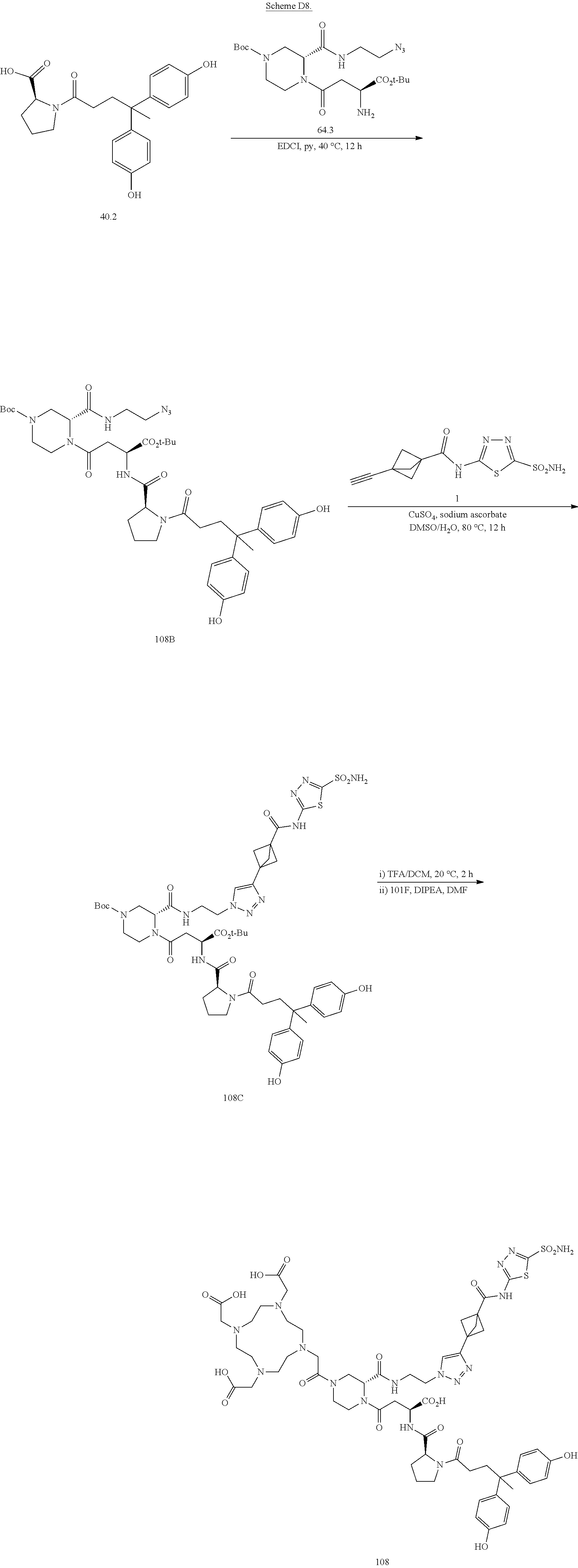

4.38 Synthesis of compound 108B

To a solution of compound 40.2 (470 mg, 1.23 mmol, 0.8 eq) and compound 64.3 in pyridine (10 mL) was added EDCl (353 mg, 1.84 mmol, 1.2 eq) at 25° C. The mixture was stirred at 40° C. for 12 h and the reaction mixture concentrated under reduced pressure. Water (20 mL) was added, the mixture extracted with EtOAc (3×50 mL), dried (Na₂SO₄), filtered and concentrated under reduced pressure. The residue was purified by column chromatography (SiO₂, Petroleum ether:EtOAc) to give compound 108B (730 mg, 57% yield) as a yellow oil; ¹H NMR (400 MHz, DMSO-d₆) δ ppm 1.15-1.28 (m, 1H) 1.33-1.41 (m, 22H) 1.49 (Br s, 2H) 1.64-1.74 (m, 1H) 1.80 (d, J=2.93 Hz, 2H) 1.98-2.01 (m, 1H) 2.15-2.23 (m, 2H) 2.67-2.74 (m, 1H) 2.87-2.95 (m, 1H) 3.13 (dd, J=12.72, 3.67 Hz, 1H) 3.17 (d, J=5.14 Hz, 1H) 3.23-3.30 (m, 2H) 3.70 (d, J=12.96 Hz, 1H) 3.99-4.12 (m, 1H) 4.19-4.33 (m, 2H) 4.45-4.57 (m, 1H) 4.70-4.79 (m, 1H) 6.59-6.69 (m, 4H) 6.89-6.98 (m, 4H) 7.90-8.04 (m, 1H) 8.12-8.23 (m, 1H) 9.11-9.18 (m, 2H).

### 4.39 Synthesis of Compound 108C

To a solution of compound 1 (214 mg, 719 μmol, 1 eq) and compound 108B (600 mg, 719 μmol, 1 eq) in DMSO (6 mL) was added a solution of CuSO₄ (5.5 μL, 36 μmol, 0.05 eq) and sodium ascorbate (142 mg, 719 μmol, leq) in H₂O (1.2 mL) at 25° C. The mixture was stirred at 80° C. for 12 h. The mixture was poured into an NH₄Cl solution (20 mL) and stirred at 25° C. for 15 min, filtered, filter cake dried and triturated with H₂O (10 mL) to give compound 108C (640 mg, 79% yield) as a yellow solid; ¹H NMR (400 MHz, DMSO-d₆) δ ppm 1.18-1.28 (m, 1H) 1.31-1.41 (m, 20H) 1.47 (br s, 2H) 1.62-1.74 (m, 1H) 1.74-1.89 (m, 3H) 1.90-2.03 (m, 2H) 2.08-2.26 (m, 2H) 2.44 (br s, 6H) 2.54 (br s, 1H) 2.63-2.77 (m, 1H) 2.86-3.08 (m, 1H) 3.10-3.22 (m, 2H) 3.62-3.82 (m, 3H) 4.01-4.15 (m, 1H) 4.17-4.29 (m, 2H) 4.29-4.41 (m, 2H) 4.43-4.57 (m, 2H) 4.62-4.73 (m, 1H) 6.59-6.69 (m, 4H) 6.93 (d, J=7.70 Hz, 4H) 7.90-8.00 (m, 1H) 8.12-8.23 (m, 1H) 8.26-8.36 (m, 2H) 9.11-9.20 (m, 2H) 12.95-13.46 (m, 1H).

### 4.40 Synthesis of Compound 108

Compound 108 was prepared from compound 10 as previously depicted; LCMS [M+H]: 1363.4.

Compounds in table 9.7 were prepared by procedures depicted in scheme D8.

TABLE 9.7

| Compound | m/z |
|---|---|
| 108.1 | [M + H]: 1168.3 |
| 108.2 | [M + H]: 1393.7 |
| 108.3 | [M + H]: 1377.7 |
| 108.4 | [M + H]: 1379.3 |
| 108.5 | [M + H]: 1363.2 |

### D9. Synthesis of Compound 109

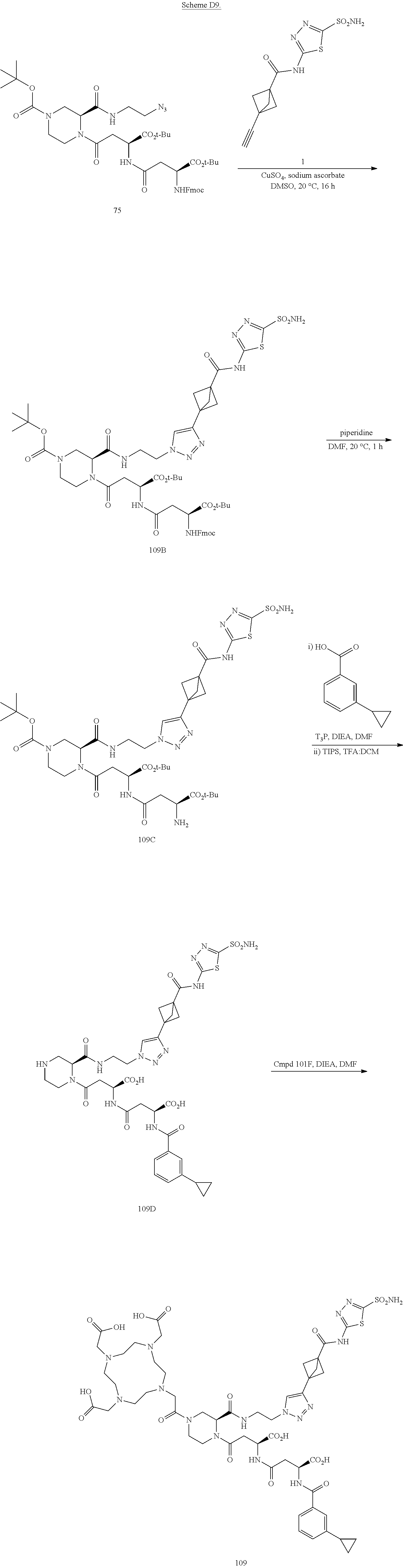

### 4.41 Synthesis of Compound 109B

To a solution of compound 75 (18 g, 20.9 mmol, 1 eq) and Intermediate 1 (5.91 g, 19.8 mmol, 0.95 eq) in DMSO (90 mL) was added CuSO₄·5H₂O (521 mg, 2.09 mmol, 0.1 eq) and sodium ascorbate (4.13 g, 20.9 mmol, 1.0 eq). The mixture was stirred at 20° C. for 16 h, poured into an NH₄Cl solution (100 g in 400 mL water), stirred for 20 min at 20° C., filtered and the filter cake dried under reduced pressure. The residue was purified by column chromatography (SiO₂, DCM:MeOH) to give compound 109B (18 g, 95% yield) as a white solid; LCMS [M+H]: 1161.4; ¹H NMR (400 MHz, METHANOL-d₄) δ 8.02-7.82 (m, 1H), 7.78 (br d, J=7.6 Hz, 2H), 7.64 (brs, 2H), 7.37 (s, 2H), 7.29 (s, 2H), 4.82-4.62 (m, 2H), 4.61-4.43 (m, 3H), 4.41-4.14 (m, 4H), 3.86-3.48 (m, 4H), 3.35 (s, 6H), 3.28-3.06 (m, 2H), 3.04-2.70 (m, 4H), 2.58-2.40 (m, 7H), 1.55-1.33 (m, 27H).

### 4.42 Synthesis of Compound 109C

To a solution of compound 109B (18 g, 19 mmol, 1 eq) in DMF (90 mL) was added piperidine (3.79 mL, 38.3 mmol, 2 eq). The mixture was stirred at 20° C. for 1 h, MTBE (90 mL) added and the residue purified by prep-TLC (SiO₂, DCM:MeOH) to give compound 109C (10.5 g, 96% yield) as a white solid; LCMS [M+H]: 939.3; ¹H NMR (400 MHz, DMSO-d₆) δ 8.73-7.55 (m, 3H), 7.51-6.02 (m, 1H), 4.83-3.95 (m, 5H), 3.91-3.25 (m, 5H), 3.25-3.09 (m, 5H), 3.03-2.59 (m, 2H), 2.37 (br s, 6H), 1.57-1.03 (m, 27H).

### 4.43 Synthesis of Compound 109D

Propylphosphonic anhydride (38 μL, 50% in DMF, 128 μmol, 1.5 eq) was added to a solution of compound 109C (80 mg, 85.2 μmol, 1.0 eq), 3-cyclopropylbenzoic acid (18 mg, 111 μmol, 1.3 eq), DIEA (37 μL, 212 μmol, 2.5 eq), and DMF (1 mL). The mixture stirred for 2 h, diluted with EtOAc, washed with water and brine, dried (Na₂SO₄), filtered and concentrated under reduced pressure. The residue was dissolved in a solution of trifluoroacetic acid (0.4 mL), tris(propan-2-yl)silane (0.4 mL) and DCM (1.2 mL) added and the reaction stirred at room temperature overnight. The mixture was concentrated under reduced pressure and the residue purified by prep-HPLC (15-35% CH₃CN in water with 0.1% TFA) to give compound 109D (40 mg, 48%) as a white powder; LCMS [M+H]: 871.3; ¹H NMR (400 MHz, DMSO) δ 13.22 (s, 1H), 12.76 (s, 2H), 8.62-8.25 (m, 5H), 8.12-7.94 (m, 2H), 7.64-7.46 (m, 2H), 7.39-7.17 (m, 2H), 5.10 (d, J=4.6 Hz, 1H), 4.74 (q, J=7.1 Hz, 1H), 4.67-4.58 (m, 1H), 4.50-4.29 (m, 3H), 3.90 (d, J=14.3 Hz, 1H), 3.73-3.42 (m, 3H), 3.27 (t, J=13.5 Hz, 2H), 3.03-2.71 (m, 4H), 2.66 (dd, J=15.5, 7.1 Hz, 1H), 2.43 (s, 6H), 1.97 (tt, J=9.1, 5.2 Hz, 1H), 1.03-0.90 (m, 2H), 0.72 (d, J=5.2 Hz, 2H).

### 4.44 Synthesis of Compound 109

Compound 109 is synthesized from compound 109D by procedures depicted in scheme D9; LCMS [M/2+H]: 629.4.

Compounds in table 9.8 were prepared by procedures depicted in Scheme D9.

TABLE 9.8

| Compound | m/z |
|---|---|
| 109.1 | [M/2 + H]: 629.3 |
| 109.2 | [M/2 + H]: 633.4 |
| 109.3 | [M/2 + H]: 645.3 |
| 109.4 | [M/2 + H]: 630.3 |
| 109.5 | [M/2 + H]: 631.2 |
| 109.6 | [M/2 + H]: 638.3 |
| 109.7 | [M/2 + H]: 629.3 |
| 109.8 | [M/2 + H]: 624.8 |
| 109.9 | [M/2 + H]: 632.4 |
| 109.10 | [M/2 + H]: 636.3 |
| 109.11 | [M/2 + H]: 645.3 |
| 109.12 | [M/2 + H]: 629.8 |
| 109.13 | [M/2 + H]: 557.3 |
| 109.14 | [M/2 + H]: 693.3 |
| 109.15 | [M/2 + H]: 638.3 |
| 109.16 | [M/2 + H]: 672.3 |
| 109.17 | [M/2 + H]: 638.7 |
| 109.18 | [M/2 + H]: 625.3 |
| 109.19 | [M/2 + H]: 629.8 |
| 109.20 | [M/2 + H]: 679.3 |
| 109.21 | [M/2 + H]: 622.2 |
| 109.22 | [M/2 + H]: 636.3 |
| 109.23 | [M/2 + H]: 630.3 |
| 109.24 | [M/2 + H]: 663.8 |
| 109.25 | [M/2 + H]: 663.8 |
| 109.26 | [M/2 + H]: 637.3 |
| 109.27 | [M/2 + H]: 644.3 |
| 109.28 | [M/2 + H]: 638.7 |
| 109.29 | [M/2 + H]: 584.3 |
| 109.30 | [M/2 + H]: 636.3 |
| 109.31 | [M/2 + H]: 637.3 |
| 109.32 | [M/2 + H]: 644.3 |
| 109.33 | [M/2 + H]: 683.4 |
| 109.34 | [M/2 + H]: 690.4 |
| 109.35 | [M/2 + H]: 643.3 |
| 109.36 | [M/2 + H]: 657.4 |
| 109.37 | [M/2 + H]: 647.3 |
| 109.38 | [M/2 + H]: 636.3 |
| 109.39 | [M/2 + H]: 630.3 |
| 109.40 | [M + H]: 1392.4 |
| 109.41 | [M/2 + H]: 672.4 |
| 109.42 | [M/2 + H]: 666.3 |
| 109.43 | [M + H]: 1317.3 |
| 109.44 | [M + H]: 1307.3 |
| 109.45 | [M/2 + H]: 665.4 |
| 109.46 | [M/2 + H]: 661.4 |
| 109.47 | [M/2 + H]: 666.3 |
| 109.48 | [M/2 + H]: 633.4 |
| 109.49 | [M/2 + H]: 673.3 |
| 109.50 | [M/2 + H]: 657.3 |
| 109.51 | [M/2 + H]: 646.3 |
| 109.52 | [M/2 + H]: 721.3 |
| 109.53 | [M/2 + H]: 647.3 |
| 109.54 | [M/2 + H]: 657.4 |
| 109.55 | [M/2 + H]: 678.4 |
| 109.56 | [M/2 + H]: 657.9 |
| 109.57 | [M/2 + H]: 660.4 |
| 109.58 | [M/2 + H]: 700.3 |
| 109.59 | [M/2 + H]: 666.8 |
| 109.60 | [M/2 + H]: 585.3 |
| 109.61 | [M/2 + H]: 636.3 |
| 109.62 | [M/2 + H]: 698.3 |
| 109.63 | [M/2 + H]: 636.4 |

### D10. Synthesis of Compound 110

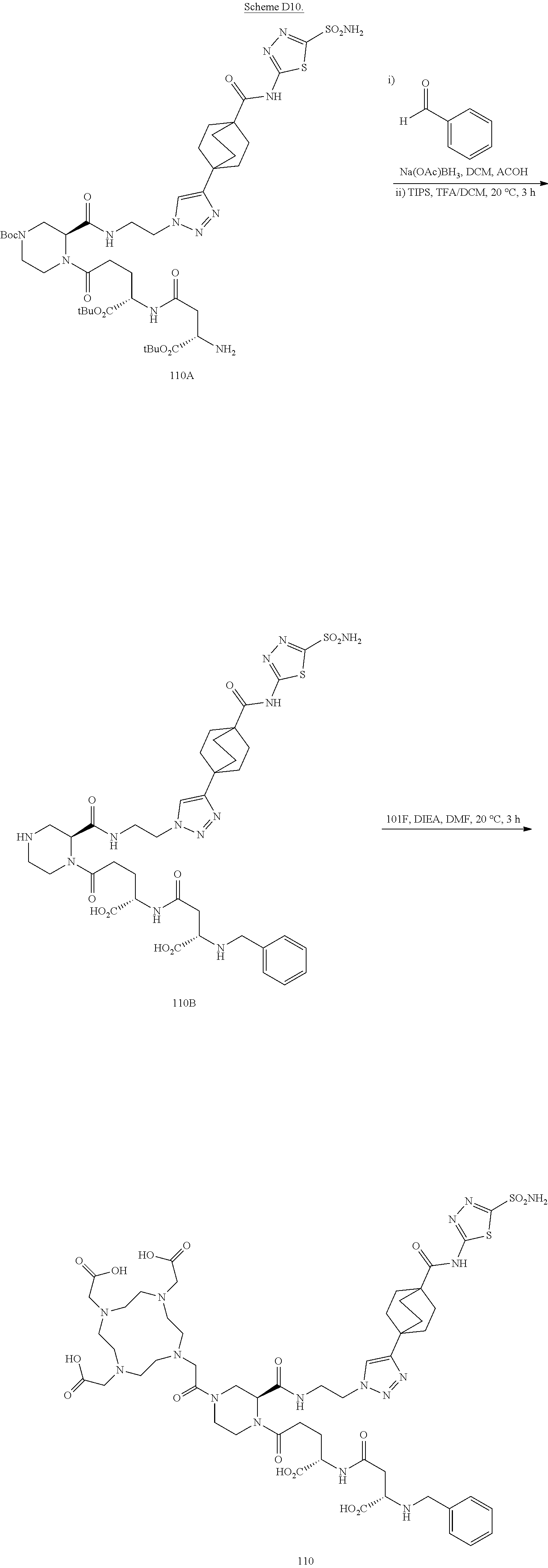 [SMILES: CC(C)(C)OC(=O)[C@H](CCC(=O)N1CCN(C(=O)OC(C)(C)C)C[C@H]1C(=O)NCCn1cc(C23CCC(C(=O)Nc4nnc(S(N)(=O)=O)s4)(CC2)CC3)nn1)NC(=O)C[C@H](N)C(=O)OC(C)(C)C ; O=Cc1ccccc1 ; NS(=O)(=O)c1nnc(NC(=O)C23CCC(c4cn(CCNC(=O)[C@@H]5CNCCN5C(=O)CC[C@H](NC(=O)C[C@H](NCc5ccccc5)C(=O)O)C(=O)O)nn4)(CC2)CC3)s1 ; NS(=O)(=O)c1nnc(NC(=O)C23CCC(c4cn(CCNC(=O)[C@@H]5CN(C(=O)CN6CCN(CC(=O)O)CCN(CC(=O)O)CCN(CC(=O)O)CC6)CCN5C(=O)CC[C@H](NC(=O)C[C@H](NCc5ccccc5)C(=O)O)C(=O)O)nn4)(CC2)CC3)s1]

### 4.45 Synthesis of Compound 110B

Benzaldehyde (20 μL, 197 μL, 2 eq) was added to a solution of compound 110A (100 mg, 100 μmol, 1 eq), DCM (2 mL), and acetic acid (0.5 mL). The mixture stirred at room temperature for 1 h, sodium triacetoxyborohydride (76 mg, 402 μL, 4 eq) added at 0° C. and the reaction stirred at room temperature overnight. The mixture diluted with EtOAc, washed with water, and saturated NaHCO₃ solution. The organic layer was dried (Na₂SO₄), filtered, and concentrated under reduced pressure. The residue was dissolved in a solution of trifluoroacetic acid (0.2 mL), tris(propan-2-yl)silane (0.2 mL), and DCM (1.2 mL) and stirred overnight. The reaction mixture was concentrated and the residue purified by prep-HPLC (10-30% CH₃CN in water with 0.1% TFA) to give compound 110B (63 mg, 72%) as a white powder; LCMS [M+H]: 873.4; ¹H NMR (400 MHz, DMSO) δ 12.71 (s, 1H), 9.30 (d, J=28.8 Hz, 3H), 8.70-8.43 (m, 3H), 8.34 (s, 2H), 8.18 (t, J=5.9 Hz, 1H), 7.84 (d, J=24.4 Hz, 1H), 7.53-7.38 (m, 5H), 5.14 (s, 1H), 4.74 (s, 1H), 4.44 (dd, J=13.7, 6.2 Hz, 2H), 4.32 (q, J=7.9 Hz, 1H), 4.21 (d, J=15.7 Hz, 3H), 3.87 (d, J=14.2 Hz, 1H), 3.66 (d, J=12.6 Hz, 1H), 3.52 (d, J=6.3 Hz, 2H), 3.34 (t, J=13.3 Hz, 1H), 3.21-2.96 (m, 3H), 2.85 (d, J=40.9 Hz, 3H), 2.43 (d, J=7.7 Hz, 1H), 2.28 (s, 1H), 2.09-1.92 (m, 6H), 1.84 (t, J=7.7 Hz, 7H).

### 4.46 Synthesis of Compound 110

Compound 110 is synthesized from compound 110B by procedures depicted in scheme D10; LCMS [M/2+H]: 630.3.

### D11. Synthesis of Compound 111

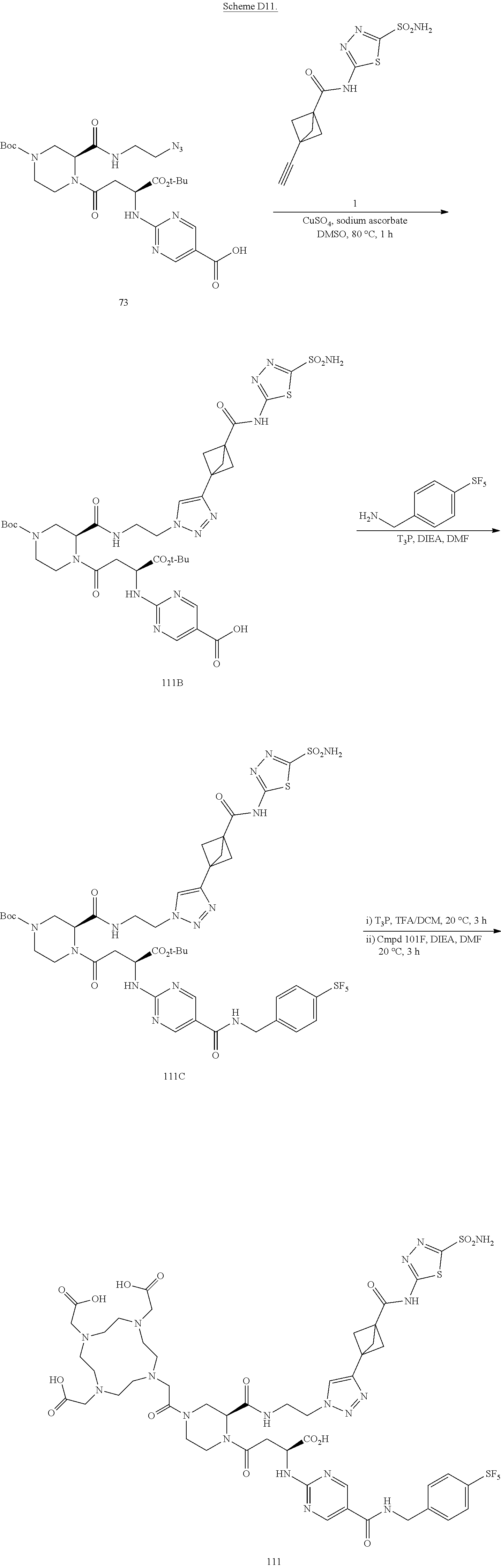

### 4.47 Synthesis of Compound 111B

To a solution of compound 73 (4.0 g, 6.76 mmol, 1 eq) and compound 1 (1.80 g, 6.02 mmol, 0.89 eq) in DMSO (40 mL) was added sodium (2R)-2-[(2R)-3,4-dihydroxy-5-oxo-2H-furan-2-yl]-2-hydroxy-ethanolate (2.68 g, 13.5 mmol, 2 eq) and CuSO₄·5H₂O (169 mg, 676 μmol, 0.1 eq). The mixture was stirred at 80° C. for 1 h, the crude product triturated with an NH₄Cl solution (200 mL H₂O, 40 g NH₄Cl) at 20° C. for 10 min, the filter cake washed with H₂O (20 mL) and the filter cake concentrated under reduced pressure. The residue was purified by column chromatography (SiO₂, DCM:MeOH to give compound 111B (4.0 g, 66% yield) as a white solid; LCMS [M+H]: 890.3.

### 4.48 Synthesis of Compound 111C

1-[4-(Pentafluoro-26-sulfanyl)phenyl]methanamine (40 mg, 170 μmol, 1.1 eq) and DIEA (81 μl, 463 μmol, 3 eq) were added to a solution of compound 111B (137 mg, 154 μmol, 1 eq) in DMF (4 mL). 1-Propanephosphonic anhydride solution (116 μl, 386 μmol, 2.5) was added dropwise, the solution stirred at room temperature for 30 min and the reaction mixture purified directly by prep-HPLC (C18, Luna 30×150 mm, 0-40% CH3CN/H2O+ 0.1% Formic acid) to give compound 111C (80 mg, 47% yield) as a white solid; [M+H]: 1105.3.

### 4.49 Synthesis of Compound 111

Compound 111 was synthesized from compound 111C by procedures depicted in scheme D11; LCMS [M/2+H]: 630.2.

Compounds in table 9.10 were prepared by procedures depicted in scheme D11.

TABLE 9

| Compound | m/z |
|---|---|
| 111.1 | [M + H]: 1359.3 |
| 111.2 | [M/2 + H]: 625.8 |
| 111.3 | [M + H]: 1249.4 |
| 111.4 | [M/2 + H]: 686.4 |
| 111.5 | [M/2 + H]: 634.8 |
| 111.6 | [M/2 + H]: 630.3 |
| 111.7 | [M/2 + H]: 646.3 |
| 111.8 | [M/2 + H]: 630.3 |
| 111.9 | [M + H]: 1227.3 |
| 111.10 | [M + H]: 1249.3 |
| 111.11 | [M/2 + H]: 632.4 |
| 111.12 | [M + H]: 1162.3 |

### D12. Synthesis of Compound 112

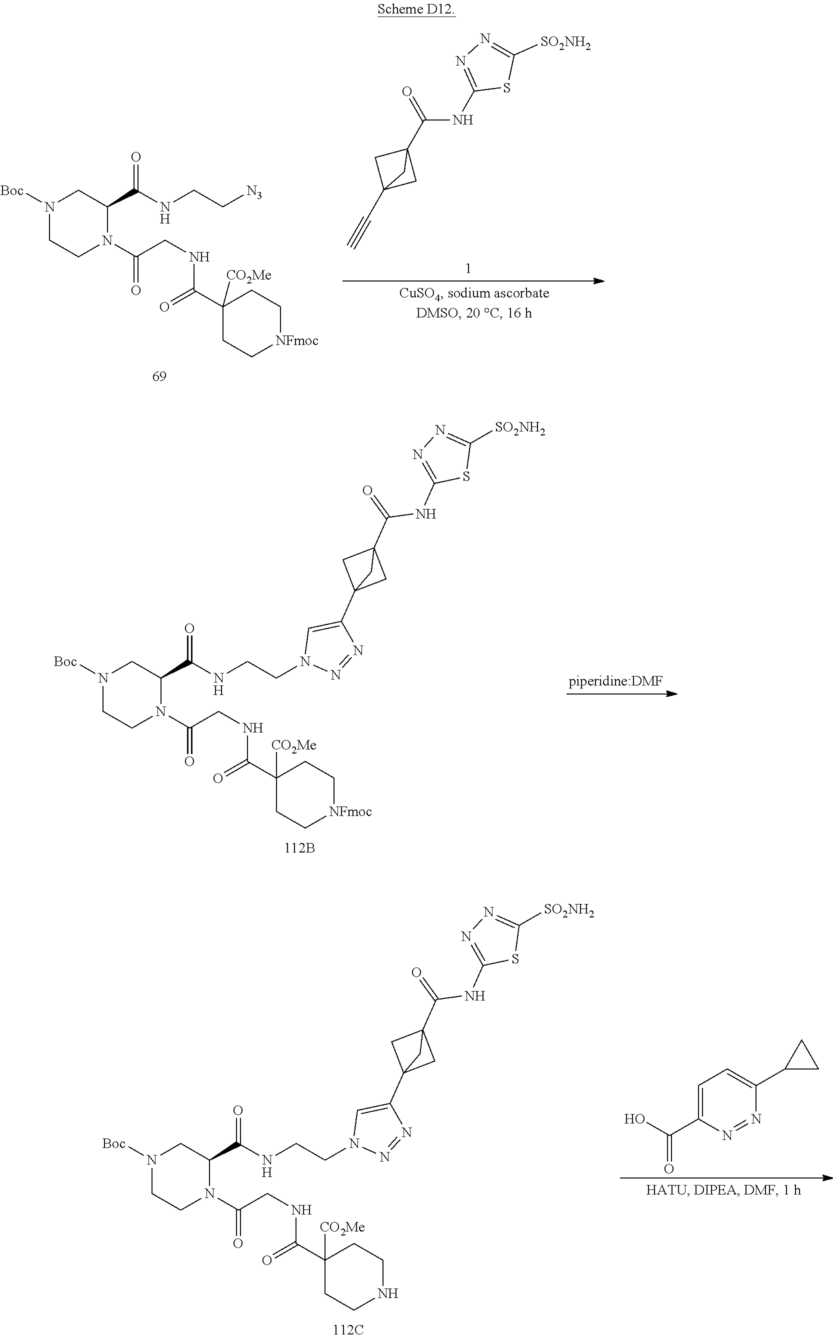

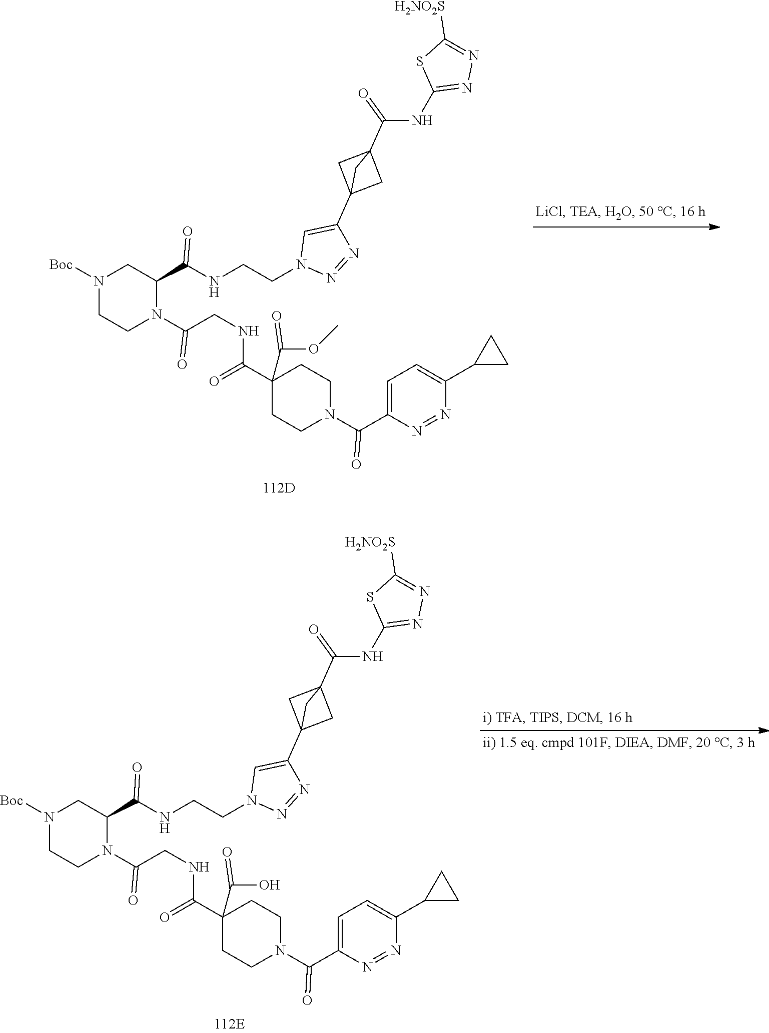 [SMILES: COC(=O)C1(C(=O)NCC(=O)N2CCN(C(=O)OC(C)(C)C)C[C@H]2C(=O)NCCn2cc(C34CC(C(=O)Nc5nnc(S(N)(=O)=O)s5)(C3)C4)nn2)CCN(C(=O)c2ccc(C3CC3)nn2)CC1 ; CC(C)(C)OC(=O)N1CCN(C(=O)CNC(=O)C2(C(=O)O)CCN(C(=O)c3ccc(C4CC4)nn3)CC2)[C@H](C(=O)NCCn2cc(C34CC(C(=O)Nc5nnc(S(N)(=O)=O)s5)(C3)C4)nn2)C1]

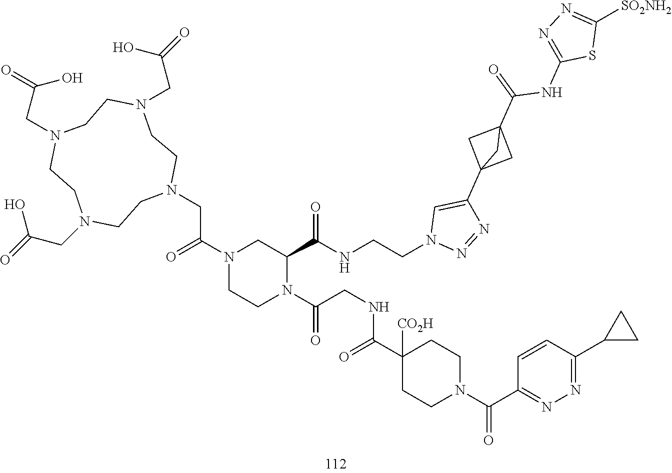 [SMILES: NS(=O)(=O)c1nnc(NC(=O)C23CC(c4cn(CCNC(=O)[C@@H]5CN(C(=O)CN6CCN(CC(=O)O)CCN(CC(=O)O)CCN(CC(=O)O)CC6)CCN5C(=O)CNC(=O)C5(C(=O)O)CCN(C(=O)c6ccc(C7CC7)nn6)CC5)nn4)(C2)C3)s1]

### 4.50 Synthesis of Intermediate 112B

To a mixture of compound 69 (4.0 g, 5.36 mmol, 1 eq) and compound 1 (1.36 g, 4.55 mmol, 0.85 eq) in DMSO (40 mL) was added CuSO₄·5H₂O (134 mg, 536 μmol, 0.1 eq) and sodium ascorbate (1.06 g, 5.36 mmol, 1 eq) in one portion at 20° C. under a nitrogen atmosphere. The mixture was stirred at 20° C. for 16 h, the reaction mixture poured into H₂O (320 mL) and aq. NH₄Cl (80 mL), and the mixture filtered. The filter cake was purified by column chromatography (SiO₂, DCM:MeOH) to give compound 112B (6.6 g, 58% yield) as a white solid; LCMS [M+H]: 1045.5.

### 4.51 Synthesis of Compound 112C

To a solution of compound 112B (7.0 g, 6.7 mmol, 1 eq) in DMF (70 mL) was added piperidine (13.2 mL, 134 mmol) and the mixture stirred at 20° C. for 1 h. MTBE (210 mL) was added, and the reaction stirred at 20° C. for 10 min. The suspension was filtered, and the filter cake concentrated under reduced pressure. The solid was purified by chromatography column (SiO₂, DCM:MeOH) to give a blue solid. The crude product was dissolved in DCM (50 mL) and MeOH (50 mL), silica gel (10 g) added and the reaction stirred at 40° C. for 0.5 h (residual copper removal), the mixture filtered and the process repeated three times. The filtrate was concentrated under reduced pressure to give compound 112C (10 g, 60% yield) as a white solid; LCMS [M+H]: 823.3; ¹H NMR (400 MHz, DMSO-d₆) δ 8.34-8.11 (m, 2H), 8.06-7.51 (m, 3H), 4.65 (br s, 1H), 4.39 (br s, 2H), 4.33-4.19 (m, 2H), 4.15-3.87 (m, 1H), 3.86-3.71 (m, 3H), 3.70-3.64 (m, 3H), 3.52 (br s, 1H), 3.24-3.08 (m, 4H), 3.04-2.91 (m, 3H), 2.40-2.26 (m, 2H), 2.25-2.15 (m, 6H), 2.13-1.95 (m, 2H), 1.71-1.48 (m, 1H), 1.37 (s, 9H).

### 4.52 Synthesis of Compound 112D

6-Cyclopropylpyridazine-3-carboxylic acid (31 mg, 190 μmol, 1.1 eq) was added to compound 112C (130 mg, 158 μmol, 1 eq) in DMF (4 mL). HATU (72 mg, 190 μmol, 1.2 eq) and DIEA (82 μl) were added dropwise, and the solution stirred at room temperature for 1 h. The reaction was purified directly by prep-HPLC (prep C18, Luna 30×150 mm, 10-60% CH3CN/H2O+ 0.1% Formic acid) to give compound 112D (65 mg, 42% yield) as a white solid; [M+H]: 969.3.

### 4.53 Synthesis of Compound 112E

Triethylamine (44 μl. 315 μmol, 3 eq) was added followed by lithium chloride (13 mg, 315 μmol, 3 eq) to a solution of compound 112D (65 mg, 63 μmol, 1 eq) in water (1 mL). The reaction solution was stirred at 50° C. for 16 h and directly purified by prep-HPLC (C18, Luna 30×150 mm, 30-50% CH₃CN:H₂O, 0.1% Formic acid) to give compound 112E (25 mg, 41%) as a white solid; [M+H]: 955.3.

### 4.54 Synthesis of Compound 112

Compound 112 was prepared from compound 112E by protocols previously depicted; LCMS [M+H]: 1241.3.

Compounds in table 9.11 were prepared by procedures depicted in scheme D12.

TABLE 9

| Compound | m/z |
|---|---|
| 112.1 | [M/2 + H]: 627.3 |
| 112.2 | [M/2 + H]: 705.1 |
| 112.3 | [M/2 + H]: 636.3 |
| 112.4 | [M/2 + H]: 634.4 |
| 112.5 | [M/2 + H]: 641.3 |
| 112.6 | [M/2 + H]: 627.3 |
| 112.7 | [M + H]: 1247.5 |
| 112.8 | [M/2 + H]: 616.3 |
| 112.9 | [M/2 + H]: 620.4 |
| 112.10 | [M/2 + H]: 620.3 |
| 112.11 | [M/2 + H]: 629.3 |
| 112.12 | [M/2 + H]: 627.4 |
| 112.13 | [M/2 + H]: 636.3 |
| 112.14 | [M/2 + H]: 636.3 |
| 112.15 | [M/2 + H]: 636.3 |
| 112.16 | [M/2 + H]: 627.3 |
| 112.17 | [M + H]: 1196.6 |
| 112.18 | [M/2 + H]: 689.4 |
| 112.19 | [M/2 + H]: 691.3 |

### D13. Synthesis of Compound 113

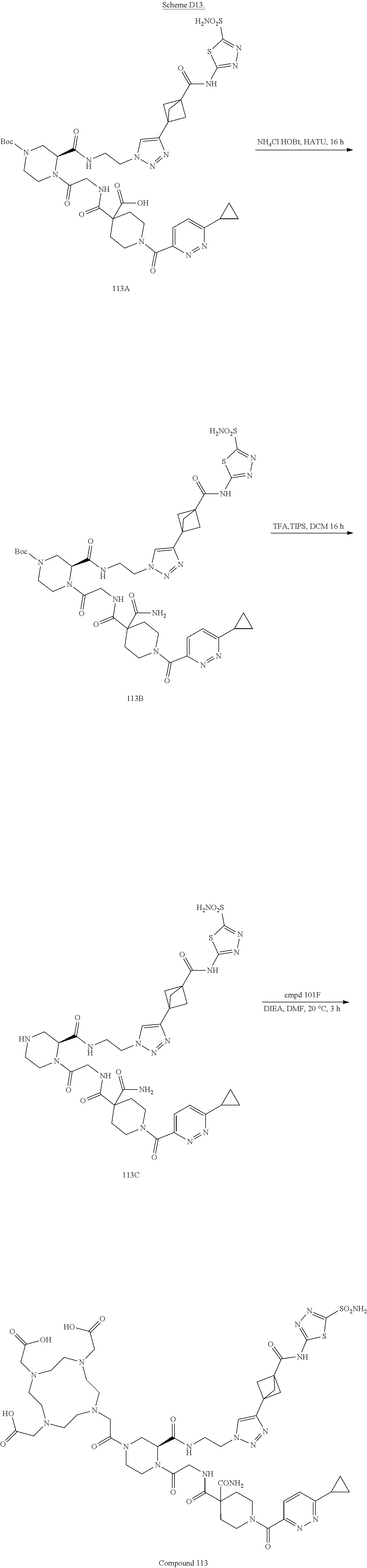

### 4.55 Synthesis of Compound 113B

Ammonium chloride (1.3 mg, 25.1 μmol, 1.2 eq), HOBt (2.0 mg, 15.1 μmol, 1.2 eq), HATU (4.7 mg, 12.6 μmol, 1 eq) and DIEA (5.5 μl, 31.4 μmol, 3 eq) were added to a solution of compound 113A (12 mg, 12.6 μmol, 1 eq) in DMF (1 mL). The solution was stirred at room temperature for 16 h and directly purified by prep-HPLC (C18, Luna 30×150 mm, 20-60% CH₃CN:H₂O+ 0.1% Formic acid) to give compound 113B (7 mg, 58% yield) as a white solid; [M+H]: 954.4.

### 4.56 Synthesis of Compound 113C

Tris(isopropyl)silane (4.5 μl, 22 μmol, 3 eq) and trifluoroacetic acid (1.7 μl, 22 μmol, 3 eq) were added to a solution of compound 113B (7.0 mg, 7.3 μmol, 1 eq) in DCM (1 mL). The reaction was stirred at room temperature for 16 h and concentrated under reduced pressure with toluene 3 times to give compound 113C (6.0 mg, 95% yield) as a white powder; [M+H]: 854.5.

### 4.57 Synthesis of Compound 113

Compound 113 was prepared from compound 113C by protocols previously depicted; LCMS [M/2+H]: 620.4.

### D14. Synthesis of Compound 114

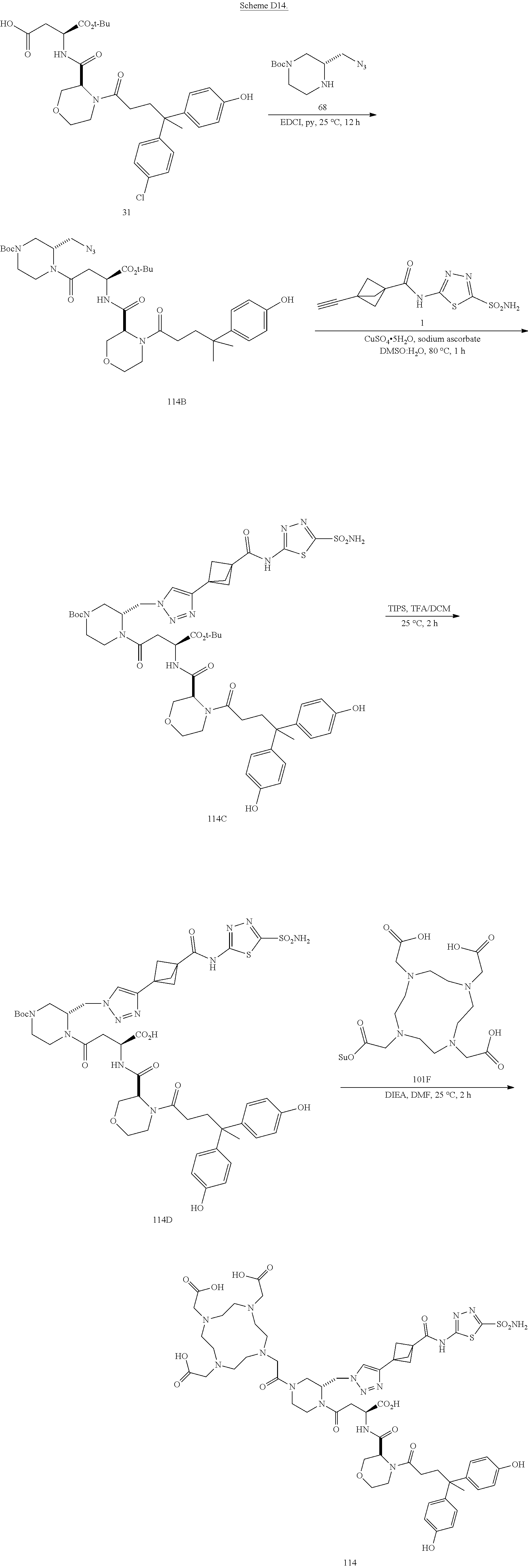

### 4.58 Synthesis of Compound 114B

To a solution of compound 68 (650 mg, 2.69 mmol, 1 eq) and compound 31 (1.54 g, 2.69 mmol, 1 eq) in pyridine (10 mL) was added EDCl (620 mg, 3.23 mmol, 1.2 eq). The mixture was stirred at 25° C. for 12 h and concentrated under reduced pressure. The residue was purified by prep-HPLC (Phenomenex Luna 80*30 mm*3 μm; mobile phase: [water (HCl)-ACN]; B %: 40%-70%, 8 min) to give compound 114B (0.91 g, 43% yield) as a white solid; LCMS [M+H]: 794.4.

### 4.59 Synthesis of Compound 114C

To a solution of compound 114B (0.86 g, 1.08 mmol, 1 eq) and compound 1 (323 mg, 1.08 mmol, 1 eq) in DMSO (10 mL) and H₂O (2 mL) was added CuSO₄·5H₂O (13.5 mg, 0.05 eq) and sodium ascorbate (230 mg, 1.1 eq). The mixture was stirred at 80° C. for 1 h, concentrated under reduced pressure and the residue purified by re-crystallization at 0° C. (H₂O) to give compound 114C (1.1 g, 93% yield) as a white solid; LCMS [M+H]: 1092.4.

### 4.60 Synthesis of Compound 14D

To a solution of compound 114C (1.00 g, 916 μmol, 1 eq) in DCM (10 mL) was added TFA (2.0 mL, 27.0 mmol, 30 eq) and TIPS (1.88 mL, 9.16 mmol, 10 eq), and the mixture stirred at 25° C. for 2 h. The reaction mixture was concentrated under reduced pressure and the crude product triturated with MTBE at 20° C. for 10 min to give compound 114D (760 mg, 89% yield) as a green solid; LCMS [M+H]: 936.3.

### 4.61 Synthesis of Compound 114

To a solution of compound 114D (760 mg, 812 μmol, 1 eq) in DMF (10 mL) was added DIEA (1.41 mL, 8.12 mmol, 10 eq) and compound 101F (937 mg, 1.87 mmol, 2.3 eq). The mixture was stirred at 25° C. for 2 h and concentrated under reduced pressure. The residue was purified by prep-HPLC (Phenomenex luna C18 100*40 mm*5 μm; mobile phase: [water (TFA)-ACN]; B %: 1%-35%, 8 min) to give Compound 114 (100 mg, 9.3% yield); LCMS [M+H]: 1322.7.

Compounds in table 9.13 were prepared by procedures depicted in scheme D14.

TABLE 9

| Compound | m/z |
|---|---|
| 114.1 | [M + H]: 1212.3 |
| 114.2 | [M + H]: 1097.3 |
| 114.3 | [M + H]: 1111.3 |

### D15. Synthesis of Compound 115

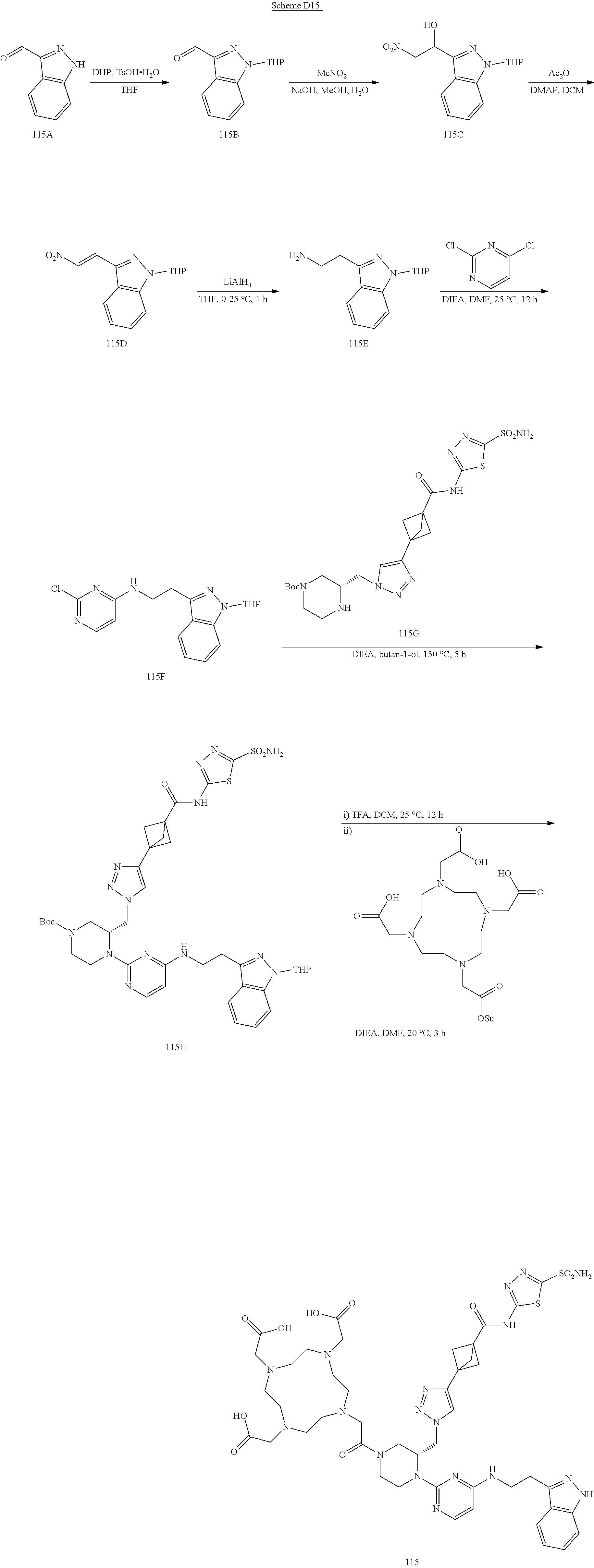

### 4.62 Synthesis of Compound 115B

To a solution of compound 115A (10 g, 68.4 mmol, 1 eq) in THF (140 mL) was added DHP (9.38 mL, 103 mmol, 1.5 eq) and TsOH·H₂O (1.30 g, 6.84 mmol, 0.1 eq), and the mixture stirred at 80° C. for 2 h. The reaction mixture was poured into ice-water (100 mL), the aqueous phase extracted with EtOAc (3×100 mL), the combined organic phase washed with brine (100 mL), dried (Na₂SO₄), filtered and concentrated under reduced pressure. The residue was purified by silica gel chromatography (SiO₂, Petroleum ether:EtOAc) to give compound 115B (12 g, 75% yield) as white oil; LCMS [M+H]: 231.1; ¹H NMR (400 MHz, CDCl₃) δ ppm 10.27 (1 H, s) 8.31 (1 H, d, J=8.19 Hz) 7.68 (1H, d, J=8.44 Hz) 7.48 (1 H, t, J=7.70 Hz) 7.37 (1H, m) 5.85 (1H, dd, J=8.93, 2.81 Hz) 4.03 (1H, m) 3.79 (1H, m) 2.59 (1H, m) 2.16 (2H, m) 1.77 (3H, m).

### 4.63 Synthesis of Compound 115C

To a solution of compound 115B (9.0 g, 39.1 mmol, 1 eq) and MeNO₂ (2.74 mL, 50.8 mmol, 1.3 eq) in MeOH (90 mL) was added a solution of NaOH (3.44 g, 86 mmol, 2.2 eq) in H₂O (9 mL) dropwise at 0° C. The reaction mixture was stirred at 0° C. for 0.5 h, quenched with 1M HCl (60 mL), the aqueous phase was extracted with EtOAc (3×100 ml), the combined organic phase washed with brine (100 mL), dried (Na₂SO₄), filtered and concentrated under reduced pressure. The residue was purified by silica gel chromatography (SiO₂, Petroleum ether/EtOAc) to give compound 115C (9.7 g, 74% yield, 87% purity) as a yellow oil; LCMS [M+H]: 292.1; ¹H NMR (400 MHz, CDCl₃) δ ppm 7.84 (1H, ddd, J=8.17, 1.85, 0.95 Hz) 7.58 (1H, dd, J=8.46, 3.81 Hz) 7.43 (1H, ddd, J=8.31, 7.12, 0.89 Hz) 7.21 (1H, m) 5.84 (1 H, ddd, J=8.76, 7.57, 3.46 Hz) 5.68 (1 H, dt, J=9.45, 2.85 Hz) 4.92 (2H, m) 4.02 (1H, m) 3.74 (1 H, td, J=10.88, 2.92 Hz) 3.16 (1H, m) 2.50 (1H, m) 2.14 (1H, m) 2.01 (1H, m) 1.72 (3H, m).

### 4.64 Synthesis of Compound 115D

To a solution of compound 115C (9.7 g, 33.3 mmol, 1 eq) in DCM (97 mL) was added Ac20 (3.12 mL, 33.3 mmol, 1 eq) and DMAP (203 mg, 1.66 mmol, 0.05 eq), and the mixture stirred at 25° C. for 2 h. The mixture was poured into ice-water (100 mL), the aqueous phase extracted with DCM (3×100 mL), the combined organic phase washed with brine (100 mL), dried (Na₂SO₄), filtered and concentrated under reduced pressure to give crude compound 115D (10 g) as a yellow solid which was used directly in the next reaction.

### 4.65 Synthesis of Compound 115E

Aa solution of compound 115D (4.0 g, 14.6 mmol, 1 eq) in THF (40 mL) was added to a stirred solution of 1M LiAlH₄ (58.6 mL, 4 eq) in THF (56 mL) dropwise at 0° C. and the reaction mixture stirred at 25° C. for 1 h. The mixture was poured into ice-water (100 mL), the aqueous phase extracted with EtOAc (3×100 mL), the combined organic phase washed with NH₄Cl solution (100 mL) and brine (100 mL), dried (Na₂SO₄), filtered and concentrated under reduced pressure to give crude compound 115E (3.2 g) as a yellow oil that was used directly in the next reaction; ¹H NMR (400 MHz, CDCl₃) δ ppm 7.68 (1 H, d, J=8.13 Hz) 7.55 (1H, d, J=8.38 Hz) 7.39 (1H, m) 7.16 (1H, m) 5.67 (1H, dd, J=9.88, 2.63 Hz) 4.07 (1H, m) 3.75 (1H, m) 3.14 (3H, m) 2.58 (1H, m) 2.10 (2H, m) 1.72 (4H, m).

### 4.66 Synthesis of Compound 115F

To a stirred solution of 2,4-dichloropyrimidine (1.94 g, 13.0 mmol, 1 eq) in anhydrous DMSO (32 mL) at 25° C. under a nitrogen atmosphere was added DIEA (4.54 mL, 26.1 mmol, 2 eq) and compound 115E (3.2 g, 13.0 mmol, 1 eq) slowly. The mixture was stirred at 25° C. for 12 h, poured into ice-water (50 mL) and extracted with EtOAc (3×20 mL). The combined organic phase was washed with brine (20 mL), dried (Na₂SO₄), filtered and concentrated under reduced pressure. The residue was purified by chromatography column (SiO₂, Petroleum ether:EtOAc) to give compound 115F (2.4 g, 43% yield, 83% purity) as a yellow solid; ¹H NMR (400 MHz, CDCl₃) δ 7.94 (1H, m) 7.66 (1 H, d, J=7.5 Hz) 7.56 (1H, d, J=8.4 Hz) 7.40 (1H, m) 7.17 (1H, t, J=7.5 Hz) 6.22 (1H, m) 5.84 (1H, br s) 5.67 (1H, dd, J=9.8, 2.51 Hz) 4.08 (1H, m) 3.78 (3H, m) 3.25 (2 H, t, J=6.5 Hz) 2.55 (1H, m) 2.16 (1H, m) 2.05 (1 H, m) 1.74 (4H, m).

### 4.67 Synthesis of Compound 115H

To a solution of compound 115G (650 mg, 1.20 mmol, 1 eq) in butan-1-ol (1.5 mL) were added DIEA (1.05 mL, 6.02 mmol, 5 eq) and compound 115F (539 mg, 1.20 mmol, 1 eq), and the mixture stirred at 150° C. for 5 h. The reaction mixture was concentrated under reduced pressure and the residue purified by prep-HPLC (column: Waters Xbridge BEH C18 250*50 mm*10 μm; mobile phase: [water (NH₄HCO₃)-ACN]; B %: 30%-60%, 10 min) to give compound 115H (270 mg, 26% yield) as a yellow solid; LCMS [M+H]: 861.2; ¹H NMR (400 MHz, CDCl₃) δ ppm 7.64 (2H, d, J=7.75 Hz) 7.52 (1 H, dd, J=8.32, 4.44 Hz) 7.38 (2H, m) 7.13 (1 H, td, J=7.38, 2.88 Hz) 5.65 (2H, d, J=10.01 Hz) 4.53 (3 H, m) 4.10 (3H, m) 3.74 (3H, m) 3.36 (1H, t, J=11.38 Hz) 3.20 (2H, m) 3.10 (1H, m) 2.96 (1H, m) 2.56 (7H, m) 2.05 (3H, m) 1.73 (4H, m) 1.53 (9 H, s).

### 4.68 Preparation of Compound 115

Compound 115 was prepared from compound 115H as previously depicted; LCMS [M+H]: 1063.3.

### D16. Synthesis of Compound 116

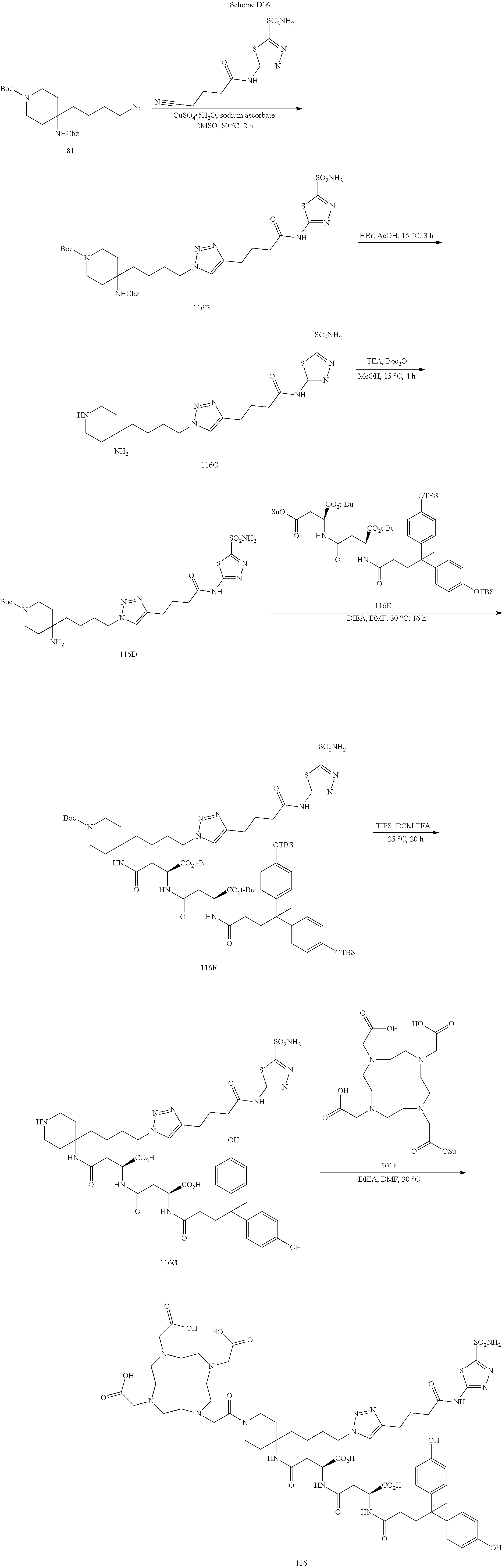

### 4.69 Synthesis of Compound 81

To a solution of compound 81 (2.0 g, 4.63 mmol, 1 eq) in DMSO (30 mL) was added N-(5-sulfamoyl-1,3,4-thiadiazol-2-yl)hex-5-ynamide (1.14 g, 4.17 mmol, 0.9 eq), sodium ascorbate (1.01 g, 5.10 mmol, 1.1 eq) and CuSO₄ (37 mg, 232 μmol, 0.05 eq) in H₂O (15 mL) at 20° C., and the mixture stirred at 80° C. for 2 h. The mixture was added to H₂O (100 mL), filtered, and the filter cake concentrated under reduce pressure to give compound 116B (2.3 g, 63% yield) as a brown solid; LCMS [M+H]: 706.2; ¹H NMR (400 MHz, METHANOL-d₄) δ 7.79-7.69 (m, 1H), 7.41-7.27 (m, 5H), 5.12-4.99 (m, 2H), 4.35-4.23 (m, 2H), 3.85-3.68 (m, 2H), 3.38-3.33 (m, 1H), 3.28-3.20 (m, 1H), 3.07-2.94 (m, 2H), 2.82-2.73 (m, 2H), 2.63-2.55 (m, 2H), 2.15-2.00 (m, 4H), 1.89-1.77 (m, 2H), 1.74-1.64 (m, 2H), 1.47-1.42 (m, 9H), 1.39-1.32 (m, 2H), 1.30-1.20 (m, 2H).

### 4.70 Synthesis of Compound 116C

A mixture of Compound 116B (5.0 g, 7.08 mmol, 1 eq) and HBr (25 mL, 33% purity) in AcOH (15 mL) was degassed and purged with nitrogen 3 times, and the mixture stirred at 15° C. for 3 h under a nitrogen atmosphere. The reaction mixture was concentrated under reduced pressure and the reside diluted with MeOH (15 mL). A basic resin was added to the mixture and stirred at 15° C. for 0.5 h. The mixture was filtered through a celite pad, the filter cake rinsed with MeOH and the filtrate concentrated under reduced pressure to give crude compound 116C (3.34 g) as a light blue solid; LCMS [M+H]: 472.2; ¹H NMR (400 MHz, METHANOL-d₄) δ 7.79 (s, 1H), 7.53-7.45 (m, 3H), 4.38 (t, J=6.8 Hz, 2H), 3.12-2.99 (m, 2H), 2.94 (dd, J=2.4, 13.0 Hz, 2H), 2.83 (s, 4H), 2.76 (t, J=7.4 Hz, 2H), 2.47 (br s, 2H), 2.10-1.99 (m, 2H), 1.63 (br s, 2H), 1.57-1.43 (m, 4H), 1.40-1.24 (m, 2H).

### 4.71 Synthesis of Intermediate 116D

To a solution of compound 116C (3.34 g, 7.08 mmol, 1 eq) in MeOH (30 mL) was added TEA (4.93 mL, 35.4 mmol, 5 eq) at 15° C. The mixture was stirred at 15° C. for 10 min, Boc₂O (1.63 mL, 7.08 mmol, 1 eq) added dropwise at 0° C. and the reaction stirred at 15° C. for 4 h. The reaction mixture was concentrated under reduced pressure and the residue purified by column chromatography (SiO₂, DCM:MeOH) to give intermediate 116D (3.2 g, 79% yield) as a light-yellow solid; LCMS [M+H]: 572.2; ¹H NMR (400 MHz, METHANOL-d₄) δ 7.78 (s, 1H), 4.37 (s, 2H), 3.63-3.44 (m, 2H), 3.39-3.35 (m, 1H), 3.34 (brs, 1H), 2.75 (t, J=7.5 Hz, 2H), 2.47 (t, J=7.4 Hz, 2H), 2.05 (t, J=7.4 Hz, 2H), 1.91 (br s, 1H), 1.87 (br s, 1H), 1.52-1.45 (m, 3H), 1.44 (s, 9H), 1.29 (s, 5H).

### 4.72 Synthesis of Intermediate 116F

To a solution of compound 116D (500 mg, 875 μmol, 1 eq) in DMF (1 mL) was added DIEA (609 μL, 3.50 mmol, 4 eq) and compound 116E (1.25 g, 1.31 mmol, 1.5 eq). The mixture was stirred at 30° C. for 16 h and concentrated under reduced pressure. The residue was purified by column chromatography (SiO₂, DCM:MeOH) to give compound 116F (155 mg, 13% yield); LCMS [M+H]: 1411.1; ¹H NMR (400 MHz, DMSO-d₆) δ 13.01 (s, 1H), 8.33 (s, 2H), 8.22 (d, J=7.7 Hz, 1H), 7.93 (d, J=7.9 Hz, 1H), 7.84 (s, 1H), 7.31 (s, 1H), 7.01 (d, J=8.6 Hz, 4H), 6.73 (d, J=8.6 Hz, 4H), 4.43-4.32 (m, 2H), 4.24 (t, J=7.1 Hz, 2H), 3.64-3.54 (m, 2H), 2.98-2.90 (m, 1H), 2.88 (brs, 1H), 2.64 (t, J=7.6 Hz, 2H), 2.61-2.57 (m, 2H), 2.48-2.39 (m, 2H), 2.23-2.18 (m, 2H), 2.10-2.00 (m, 2H), 1.96-1.91 (m, 2H), 1.89-1.84 (m, 2H), 1.58 (d, J=7.9 Hz, 6H), 1.50 (s, 3H), 1.36-1.34 (m, 27H), 1.22 (d, J=6.1 Hz, 2H), 1.15-1.10 (m, 2H), 0.93 (s, 18H), 0.16-0.16 (m, 12H).

### 4.73 Synthesis of Compound 116G

To a solution of compound 116F (150 mg, 106 μmol, 1 eq) in DCM (1 mL) was added TIPS (131 μL, 638 μmol, 6 eq) and TFA (1 mL). The mixture was stirred at 15° C. for 16 h, TFA (1 mL) added at 15° C., the mixture stirred at 25° C. for 20 h and concentrated under reduced pressure. The residue was triturated with MTBE at 15° C. for 10 min to give crude compound 116G (130 mg) as a white solid; LCMS [M+H]: 970.5; ¹H NMR (400 MHz, DMSO-d₆) δ 13.02 (s, 1H), 8.44-8.37 (m, 1H), 8.34 (s, 2H), 8.28 (d, J=7.7 Hz, 1H), 7.93 (d, J=8.1 Hz, 1H), 7.85 (s, 1H), 7.52 (s, 1H), 6.92 (d, J=8.3 Hz, 4H), 6.64 (d, J=8.6 Hz, 4H), 4.53-4.42 (m, 2H), 4.30-4.20 (m, 2H), 3.13-3.08 (m, 2H), 2.99 (brd, J=1.3 Hz, 2H), 2.69-2.56 (m, 8H), 2.48-2.37 (m, 2H), 2.27-2.11 (m, 4H), 1.97-1.91 (m, 2H), 1.89-1.81 (m, 2H), 1.77-1.58 (m, 4H), 1.45 (s, 3H), 1.26-1.12 (m, 2H), 1.05-1.03 (m, 2H), 0.96-0.86 (m, 2H).

### 4.74 Synthesis of Compound 116

To a solution of compound 116G (110 mg, 113 μmol, 1 eq) in DMF (1 mL) were added DIEA (198 μL, 1.13 mmol, 10 eq) and compound 101F (57 mg, 113 μmol, 1 eq). The reaction mixture was stirred at 30° C. for 6 h and concentrated under reduced pressure. The residue was purified by prep-HPLC (TFA condition: column: Phenomenex Luna C18 150*30 mm*5 μm; mobile phase: [water (TFA)-ACN] B %: 15%-35% 8 min) to give compound 116 (45 mg, 29% yield); LCMS [M+H]: 1356.3; ¹H NMR (400 MHz, DMSO-d₆) δ 13.00 (br s, 1H), 12.91-12.28 (m, 3H), 9.29-9.05 (m, 1H), 8.32 (s, 2H), 8.23 (d, J=7.7 Hz, 1H), 7.94-7.89 (m, 1H), 7.83 (s, 1H), 7.48 (brs, 1H), 6.92 (d, J=8.2 Hz, 4H), 6.64 (d, J=8.5 Hz, 4H), 4.53-4.44 (m, 2H), 4.28-4.17 (m, 4H), 4.11 (br s, 2H), 4.00 (d, J=11.0 Hz, 1H), 3.61 (d, J=3.3 Hz, 2H), 3.51-3.34 (m, 8H), 3.28 (d, J=4.3 Hz, 2H), 3.17-2.93 (m, 10H), 2.88-2.79 (m, 1H), 2.69-2.54 (m, 8H), 2.41-2.36 (m, 2H), 2.35-2.30 (m, 1H), 2.21-2.08 (m, 4H), 1.99-1.91 (m, 2H), 1.89-1.81 (m, 2H), 1.77-1.68 (m, 2H), 1.65 (d, J=6.9 Hz, 2H), 1.45 (s, 3H), 1.34 (t, J=10.0 Hz, 1H), 1.27-1.10 (m, 3H).

Compounds in table 9.14 were prepared by procedures previously depicted.

TABLE 9

| Compound | m/z |
|---|---|
| 116.1 | [M + H]: 1501.3 |
| 116.2 | [M + H]: 1487.3 |
| 116.3 | [M + H]: 1443.3 |
| 116.4 | [M + H]: 1473.3 |

### D17. Synthesis of Compound 117

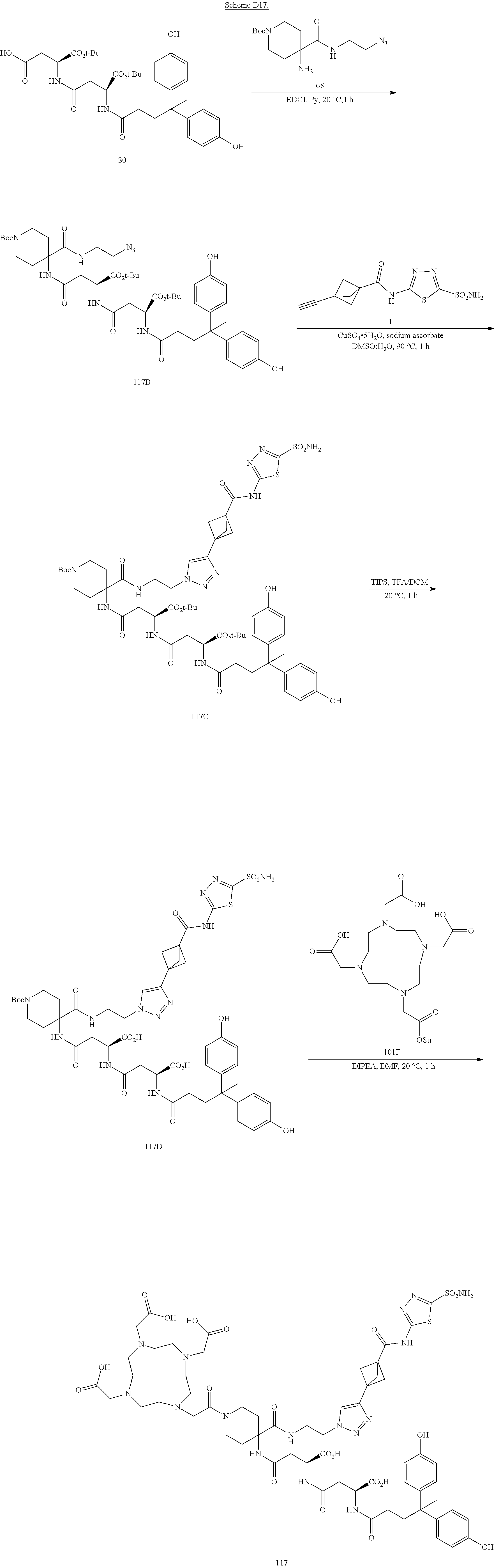

### 4.75 Synthesis of Compound 117B

To a solution of compound 68 (160 mg, 512 μmol, 1 eq) in pyridine (3.5 mL) was added compound 30 (354 mg, 563 μmol, 1.1 eq) and EDCl (196 mg, 1.02 mmol, 2 eq) and the reaction stirred at 20° C. for 1 h. The reaction mixture was concentrated under reduced pressure and the residue purified by column chromatography (SiO₂, DCM:MeOH) to give compound 117B (360 mg, 76% yield) as a white solid; ¹H NMR (400 MHz, CHLOROFORM-d) δ 7.14-6.83 (m, 7H), 6.81-6.62 (m, 5H), 4.90-4.57 (m, 2H), 3.94-3.77 (m, 2H), 3.54-3.31 (m, 4H), 3.12-2.98 (m, 2H), 2.76-2.58 (m, 3H), 2.48-2.27 (m, 3H), 2.06-1.90 (m, 6H), 1.70 (br s, 4H), 1.50-1.38 (m, 27H).

### 4.76 Synthesis of Compound 117C

To a solution of compound 117B (290 mg, 314 μmol, 1 eq) and compound 1 (94 mg, 314 μmol, 1 eq) in DMSO (3 mL) was added CuSO₄·5H₂O (16 mg, 63 μmol, 0.2 eq) and sodium ascorbate (124 mg, 628 μmol, 2 eq). The reaction mixture was stirred at 90° C. for 1 h, poured into a H₂O (12 mL) and NH₄Cl solution (3 mL), and stirred for 20 min. The mixture was filtered and the solid concentrated under reduced pressure. The crude product was washed with MTBE (2×5 mL) to give crude compound 117C (160 mg) as a white solid; ¹H NMR (400 MHz, MeOH-d₄) δ 7.93 (br s, 1H), 7.09-6.93 (m, 4H), 6.74-6.59 (m, 4H), 4.70-4.44 (m, 5H), 3.88-3.74 (m, 3H), 3.69-3.47 (m, 3H), 3.16-2.98 (m, 2H), 2.87-2.44 (m, 10H), 2.39-2.28 (m, 2H), 2.15-1.94 (m, 3H), 1.93-1.79 (m, 3H), 1.61-1.50 (m, 4H), 1.47-1.36 (m, 27H).

### 4.77 Synthesis of Compound 117D

To a solution of compound 117C (160 mg, 131 μmol, 1 eq) in DCM (2.5 mL) and TFA (1 mL) was added triisopropylsilane (538 μL, 2.62 mmol, 20 eq) at 20° C., and the mixture stirred at 20° C. for 1 h. The reaction mixture was concentrated under reduced pressure, the residue triturated with MTBE (2 mL), the suspension filtered, and the filter cake washed with MTBE (2×1 mL). The filter cake was concentrated under reduced pressure to give compound 117D (130 mg, 98% yield) as a green solid; 1H NMR (400 MHz, MeOH-d₄) δ 7.00 (br d, J=8.6 Hz, 4H), 6.66 (br d, J=8.6 Hz, 4H), 4.58-4.45 (m, 1H), 3.67-3.58 (m, 2H), 2.66 (s, 9H), 2.54 (br d, J=8.6 Hz, 6H), 2.43 (s, 4H), 2.34 (br dd, J=6.4, 9.3 Hz, 2H), 2.27-2.20 (m, 3H), 2.04 (br dd, J=8.6, 11.8 Hz, 2H), 1.59-1.48 (m, 4H), 1.19 (s, 6H).

### 4.78 Synthesis of Compound 117

To a solution of compound 117D (130 mg, 129 μmol, 1 eq) in DMF (1 mL) was added DIEA (224 μL, 1.29 mmol, 10 eq) and compound 101F (226 mg, 451 μmol, 3.5 eq) under a nitrogen atmosphere. The mixture was stirred at 20° C. for 1 h, the reaction mixture quenched by the addition of H₂O (1 mL) and concentrated under reduced pressure. The crude product was purified by prep-HPLC (column: Phenomenex luna C18 100*40 mm*5 μm; mobile phase: [water (TFA)-ACN]; gradient: 5%-35% B over 8 min) to give Compound 117 (19 mg, 11% yield); LCMS [M/2+H]: 698.5; ¹H NMR (400 MHz, DMSO-d₆) δ 9.19-9.12 (m, 1H), 8.34 (s, 2H), 8.31-8.25 (m, 1H), 8.20-8.10 (m, 1H), 7.93 (s, 2H), 7.77-7.67 (m, 1H), 6.92 (d, J=7.8 Hz, 4H), 6.64 (d, J=8.6 Hz, 4H), 4.49 (br d, J=6.4 Hz, 2H), 4.35 (br s, 2H), 4.21-3.92 (m, 4H), 3.71-3.49 (m, 8H), 3.16 (s, 12H), 3.12-2.95 (m, 10H), 2.66 (s, 4H), 2.42 (s, 7H), 2.36-2.35 (m, 1H), 2.32 (s, 1H), 2.17 (br dd, J=6.8, 9.2 Hz, 2H), 2.02-1.91 (m, 2H), 1.90-1.78 (m, 3H), 1.77-1.65 (m, 1H), 1.45 (s, 3H).

Compounds in table 9.15 were prepared by procedures depicted in scheme D17.

TABLE 9

| Compound | m/z |
|---|---|
| 117.1 | [M + H]: 1391.6 |
| 117.2 | [M/2 + H]: 555.3 |
| 117.3 | [M + H]: 1407.7 |
| 117.4 | [M + H]: 1391.5 |
| 117.5 | [M/2 + H]: 694.0 |
| 117.6 | [M/2 + H]: 715.1 |
| 117.7 | [M + H]: 1415.0 |
| 117.8 | [M + H]: 1360.9 |

### D18. Synthesis of Compound 118

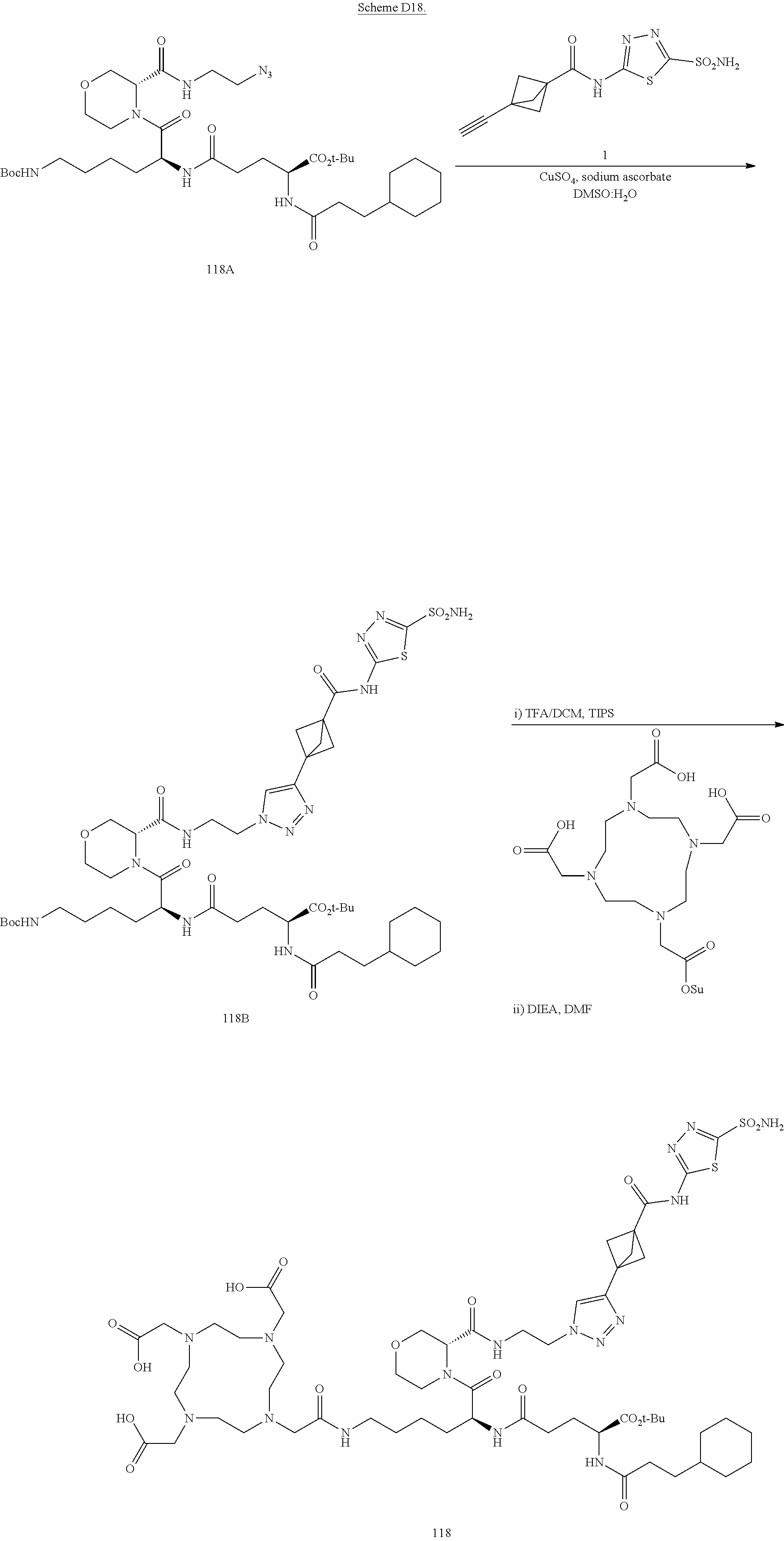

### 4.79 Preparation of Compound 118B

To a solution of compound 118A (1.1 g, 1.03 mmol, 70% purity, 1 eq) and compound 1 (306 mg, 1.03 mmol, 1 eq) in DMSO (20 mL) was added a solution of sodium ascorbate (203 mg, 1.03 mmol, 1 eq) and CuSO₄·5H₂O (25.6 mg, 103 μmol, 0.1 eq) in H₂O (4 mL) in one portion at 20° C. The mixture was stirred at 80° C. for 1 h and triturated with water at 20° C. for 5 min to give compound 118B (730 mg, 68% yield) as a green solid; LCMS [M+H]: 1049.2.

### 4.80 Preparation of Compound 118

Compound 118 was prepared from compound 118B as previously depicted; LCMS [M+H]: 1279.5; ¹H NMR (400 MHz, DMSO-d₆) δ 0.78-0.88 (m, 2H) 1.09-1.31 (m, 6H) 1.34-1.50 (m, 5H) 1.53-1.78 (m, 8H) 1.87-1.98 (m, 1H) 2.09-2.22 (m, 4H) 2.40-2.45 (m, 6H) 3.09 (br s, 11H) 3.27-3.41 (m, 12H) 3.70-3.95 (m, 7H) 4.02-4.33 (m, 5H) 4.37-4.57 (m, 3H) 4.64-4.72 (m, 1H) 7.84-7.92 (m, 1H) 8.03-8.09 (m, 1H) 8.12-8.26 (m, 1H) 8.30-8.37 (m, 3H) 8.40-8.47 (m, 1H) 13.15-13.28 (m, 1H).

Compounds in table 9.16 were prepared by procedures depicted in Scheme D18.

TABLE 9

| Compound | m/z |
|---|---|
| 118.1 | [M + H]: 1279.7 |
| 118.2 | [M + H]: 1322.7 |
| 118.3 | [M + H]: 1285.7 |
| 118.4 | [M + H]: 1285.7 |

### D19. Synthesis of Compound 119

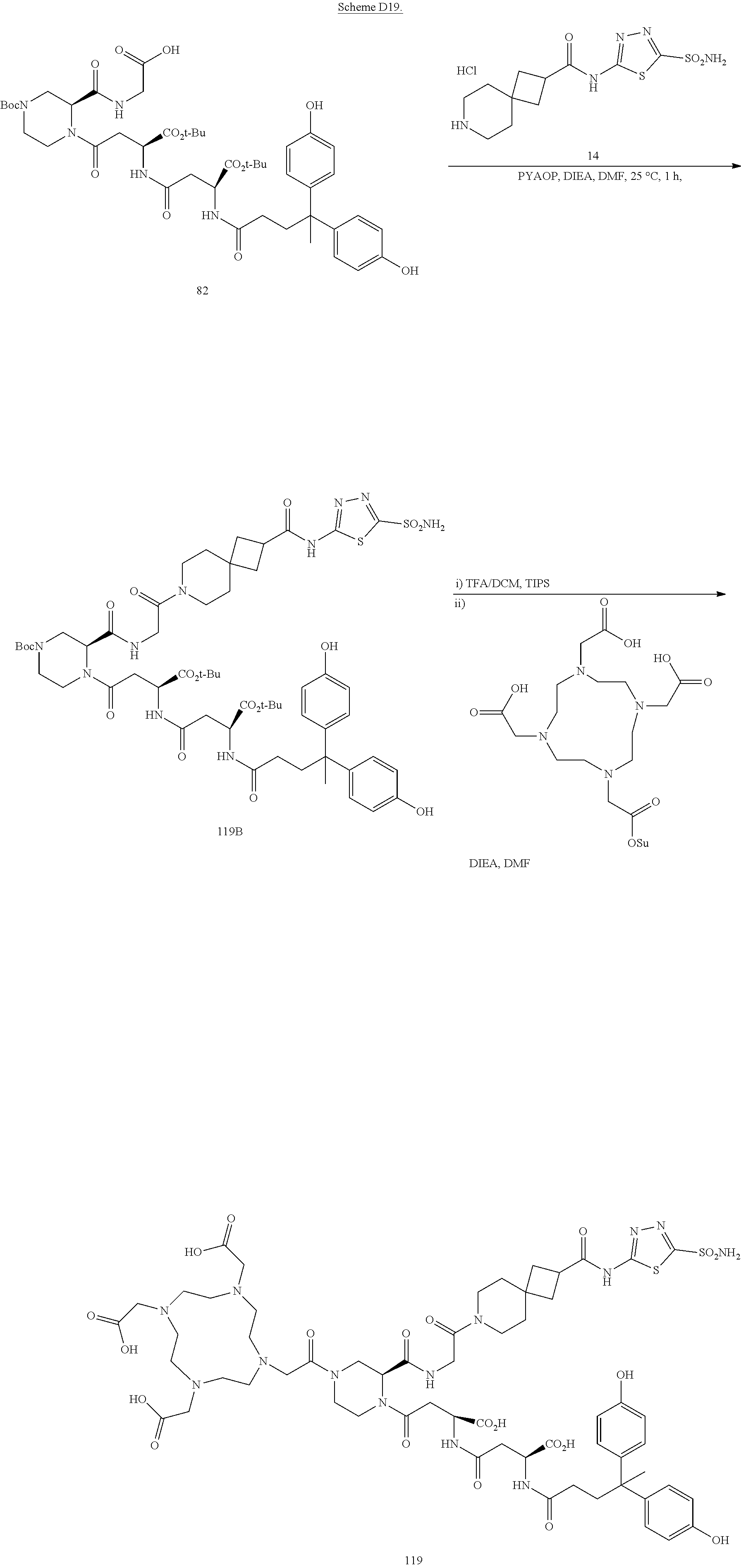

### 4.81 Preparation of Compound 119B

To a solution of compound 14 (294 mg, 668 μmol, 1 eq, HCl) in DMF (7 mL) was added DIEA (349 μL, 2.00 mmol, 3 eq), compound 82 (0.60 g, 668 μmol, 1 eq), and PyAOP (383 mg, 735 μmol, 1.1 eq). The mixture was stirred at 25° C. for 1 h. The reaction was repeated and combined. The reaction mixture was poured into H₂O (100 mL), the aqueous layer extracted with EtOAc (3×30 mL), the combined organic layers were washed with brine (100 mL), dried (Na₂SO₄), filtered and concentrated under reduced pressure. The residue was purified by chromatography column (EtOAc:MeOH) to give compound 118B (1.0 g, 54% yield, 87% purity) as a white solid.

### 4.82 Preparation of Compound 119

Compound 119 was prepared from compound 119B as previously depicted; LCMS [M+H]: 1385.5; ¹H NMR (400 MHz, DMSO-d₆) δ 13.11-12.29 (m, 3H), 9.30-9.09 (m, 1H), 8.39-8.01 (m, 3H), 8.00-7.80 (m, 2H), 6.93 (d, J=8.3 Hz, 4H), 6.64 (d, J=8.5 Hz, 4H), 5.11-4.83 (m, 1H), 4.73-4.38 (m, 3H), 4.29-3.53 (m, 16H), 3.19-2.80 (m, 19H), 2.69-2.60 (m, 3H), 2.37-2.28 (m, 1H), 2.21-1.96 (m, 7H), 1.90-1.81 (m, 2H), 1.62-1.37 (m, 7H).

### D20. Synthesis of Compound 120

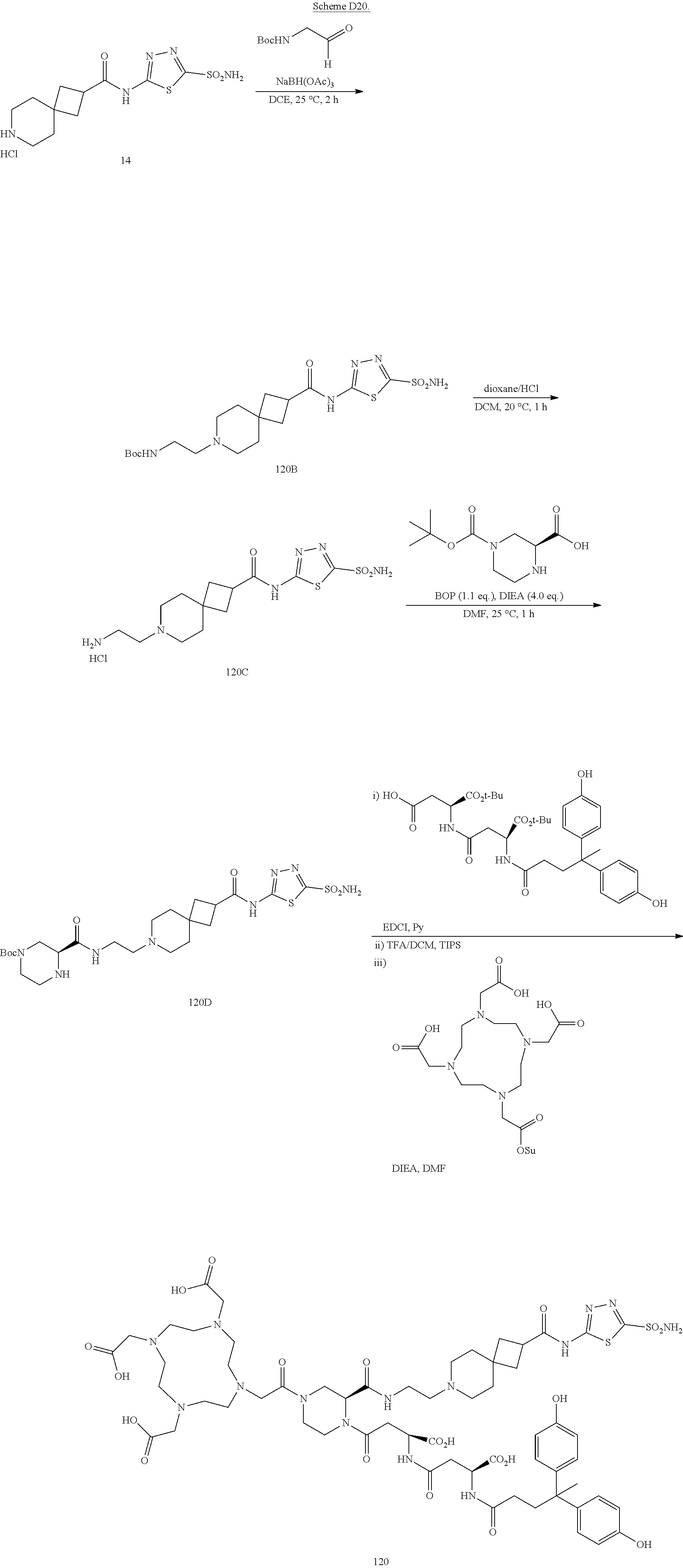

### 4.86 Preparation of Compound 120B

To a solution of compound 14 (25.7 g, 69.9 mmol, 1 eq, HCl salt) in DCE (257 mL) was added a solution of tert-butyl(2-oxoethyl) carbamate (13.3 g, 83.8 mmol, 1.2 eq) in DCE (64 mL) at 0° C. NaBH(OAc)₃ (22.2 g, 105 mmol, 1.5 eq) was added, the mixture stirred at 25° C. for 2 h and poured into saturated aqueous NaHCO₃ (100 mL). The organic layer was separated, the aqueous layer extracted with DCM (3×100 mL), the combined organic layers washed with brine (3×100 mL), dried (Na₂SO₄), filtered and concentrated under reduced pressure. The residue was purified by chromatography column (EtOAc:MeOH) to give compound 120B (12 g, 36% yield) as a light-yellow solid.

### 4.87 Preparation of Compound 120C

To a solution of compound 120B (9.5 g, 20.0 mmol, 1 eq) in DCM (100 mL) was added 4M HCl in dioxane (95 mL), the mixture stirred at 20° C. for 1 h and concentrated under reduced pressure to give crude compound 120C (8.5 g, HCl salt) as a white solid. The product was used directly.

### 4.88 Preparation of Compound 120D

To a solution of compound 120C (2.4 g, 5.84 mmol, 1 eq, HCl) in DMF (48 mL) was added DIEA (4.07 mL, 23.4 mmol, 4 eq), (S)-4-(tert-butoxycarbonyl) piperazine-2-carboxylic acid (1.34 g, 5.84 mmol, 1 eq) and BOP (2.84 g, 6.42 mmol, 1.1 eq). The mixture was stirred at 25° C. for 1 h. The reaction was repeated twice and the 3 reaction mixtures combined, filtered and the filtrate purified by prep-HPLC (column: Welch Xtimate C18 180*70 mm #10 μm; mobile phase: [H₂O (0.1% TFA)-ACN]; gradient: 5%-35% B over 20.0 min) to give compound 120D (3.0 g, 29% yield) as a white solid; LCMS [M+H]: 587.4.

### 4.89 Preparation of Compound 120

Compound 120 was prepared from Compound 120D as previously depicted; LCMS [M+H]: 1371.4.

### D21. Synthesis of Compound 121

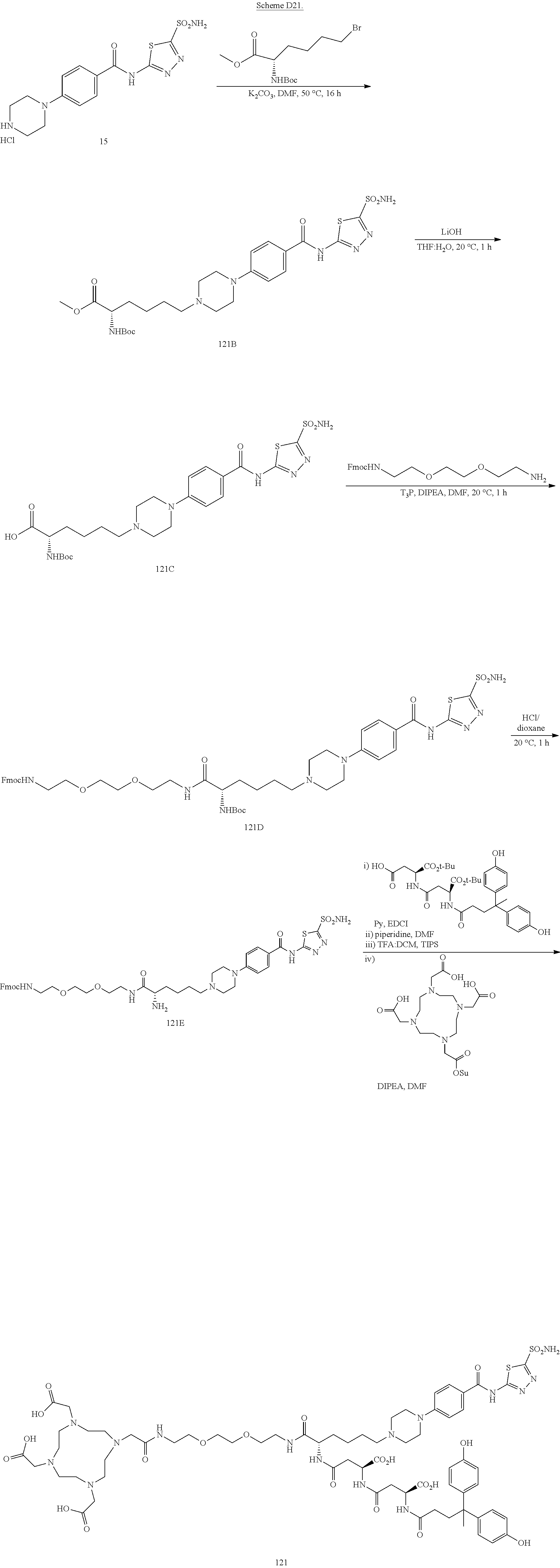

### 4.90 Preparation of Compound 121B

To a solution of compound 15 (1.89 g, 5.13 mmol, 1 eq) in DMF (20 mL) was added K₂CO₃ (2.84 g, 20.5 mmol, 4 eq) and methyl(S)-6-bromo-2-((tert-butoxycarbonyl)amino) hexanoate (1.83 g, 5.64 mmol, 1.1 eq). The mixture was stirred at 50° C. for 16 h, the aqueous phase acidified to pH=7 with aqueous HCl and the aqueous phase extracted with 5:1 DCM:IPA (2×30 mL). The combined organic phase was dried (Na₂SO₄), filtered and concentrated under reduced pressure. The residue was purified by column chromatography (SiO₂, DCM:MeOH) to give compound 121B (813 mg, 23% yield, 90% purity) as a white solid; LCMS [M+H]: 612.3; ¹H NMR (400 MHz, DMSO-d₆) δ 13.09-12.59 (m, 1H), 8.26 (s, 2H), 8.05 (d, J=9.0 Hz, 2H), 7.24 (d, J=7.8 Hz, 1H), 7.03 (d, J=9.0 Hz, 2H), 3.99-3.90 (m, 1H), 3.62 (s, 3H), 3.37 (br s, 4H), 3.17 (d, J=5.2 Hz, 5H), 2.41-2.28 (m, 2H), 1.73-1.54 (m, 2H), 1.41-1.26 (m, 12H).

### 4.91 Preparation of Compound 121C

To a solution of compound 121B (710 mg, 1.16 mmol, 1 eq) in THF (2 mL) and H₂O (2 mL) was added LiOH (83 mg, 3.48 mmol, 3 eq). The mixture was stirred at 20° C. for 1 h and concentrated under reduced pressure. The aqueous layer was acidified to pH=5 with 1N HCl, filtered and the filter cake concentrated under reduced pressure to give crude compound 121C (852 mg) as a white solid; LCMS [M+H]: 598.3.

¹H NMR (400 MHz, DMSO-d₆) δ 13.14-12.03 (m, 1H), 8.28 (s, 2H), 8.11-8.01 (m, 2H), 7.06 (br d, J=8.8 Hz, 2H), 3.86 (dt, J=5.0, 8.4 Hz, 1H), 3.50 (br d, J=1.4 Hz, 8H), 2.81 (br s, 3H), 2.69-2.54 (m, 2H), 1.75-1.48 (m, 3H), 1.43-1.30 (m, 9H).

### 4.92 Preparation of Compound 121D

To a solution of compound 121C (791 mg, 1.32 mmol, 1 eq) in DMF (8 mL) was added (9H-fluoren-9-yl)methyl(2-(2-(2-aminoethoxy)ethoxy)ethyl)carbamate (538 mg, 1.32 mmol, 1 eq), DIPEA (576 μL, 3.31 mmol, 2.5 eq) and T₃P (4.73 mL, 7.94 mmol, 50% purity, 6 eq). The mixture was stirred at 20° C. for 1 h, the reaction mixture quenched by addition of H₂O (10 mL) at 25° C. and concentrated under reduced pressure. The residue was diluted with H₂O (10 mL) and extracted with EtOAc (20 mL), the combined organic layers washed with brine (10 mL), dried (Na₂SO₄), filtered and concentrated under reduced pressure. The residue was purified by column chromatography (SiO₂, DCM:MeOH) to give compound 121D (300 mg, 23% yield, 95% purity) as a white solid; LCMS [M+H]: 950.4; ¹H NMR (400 MHz, DMSO-d₆) δ 13.15-12.69 (m, 1H), 8.26 (s, 2H), 8.04 (d, J=9.0 Hz, 2H), 7.88 (d, J=7.4 Hz, 2H), 7.81 (t, J=5.6 Hz, 1H), 7.68 (d, J=7.4 Hz, 2H), 7.44-7.38 (m, 2H), 7.36-7.28 (m, 3H), 7.01 (br d, J=9.0 Hz, 2H), 6.77 (br d, J=8.2 Hz, 1H), 4.29 (d, J=6.8 Hz, 2H), 4.23-4.17 (m, 1H), 3.93-3.84 (m, 1H), 3.50 (s, 4H), 3.44-3.33 (m, 9H), 3.28-3.20 (m, 1H), 3.20-3.09 (m, 3H), 2.39-2.28 (m, 2H), 1.65-1.54 (m, 1H), 1.53-1.40 (m, 3H), 1.37 (s, 9H), 1.31-1.19 (m, 2H).

### 4.93 Preparation of Compound 121E

4M HCl/dioxane (4 mL) was added to a solution of compound 121D (187 mg, 197 μmol, 1 eq) in dioxane (4 mL) and the mixture stirred at 20° C. for 1 h. The reaction mixture was concentrated under reduced pressure to give crude compound 121E (257 mg) as a white solid; LCMS [M+H]: 850.4; ¹H NMR (400 MHz, DMSO-d₆) δ 13.27-13.11 (m, 1H), 11.34 (br s, 1H), 8.68 (br t, J=5.4 Hz, 1H), 8.37-8.26 (m, 5H), 8.10 (brd, J=8.8 Hz, 2H), 7.89 (d, J=7.4 Hz, 2H), 7.69 (d, J=7.4 Hz, 2H), 7.45-7.38 (m, 2H), 7.38-7.29 (m, 3H), 7.16-7.09 (m, 2H), 4.29 (br d, J=6.8 Hz, 2H), 4.23-4.18 (m, 1H), 4.10 (br d, J=13.0 Hz, 2H), 3.83-3.72 (m, 1H), 3.52 (s, 5H), 3.49-3.36 (m, 6H), 3.20-3.01 (m, 6H), 1.86-1.68 (m, 4H), 1.41-1.28 (m, 2H).

### 4.94 Preparation of Compound 121

Compound 121 was prepared from compound 121E as previously depicted; LCMS [M+H]: 1512.6.

Compounds in table 9.17 were prepared by procedures depicted in Scheme D21.

TABLE 9

| Compound | m/z |
|---|---|
| 121.1 | [M + H]: 1511.7 |

### D22. Synthesis of Compound 122

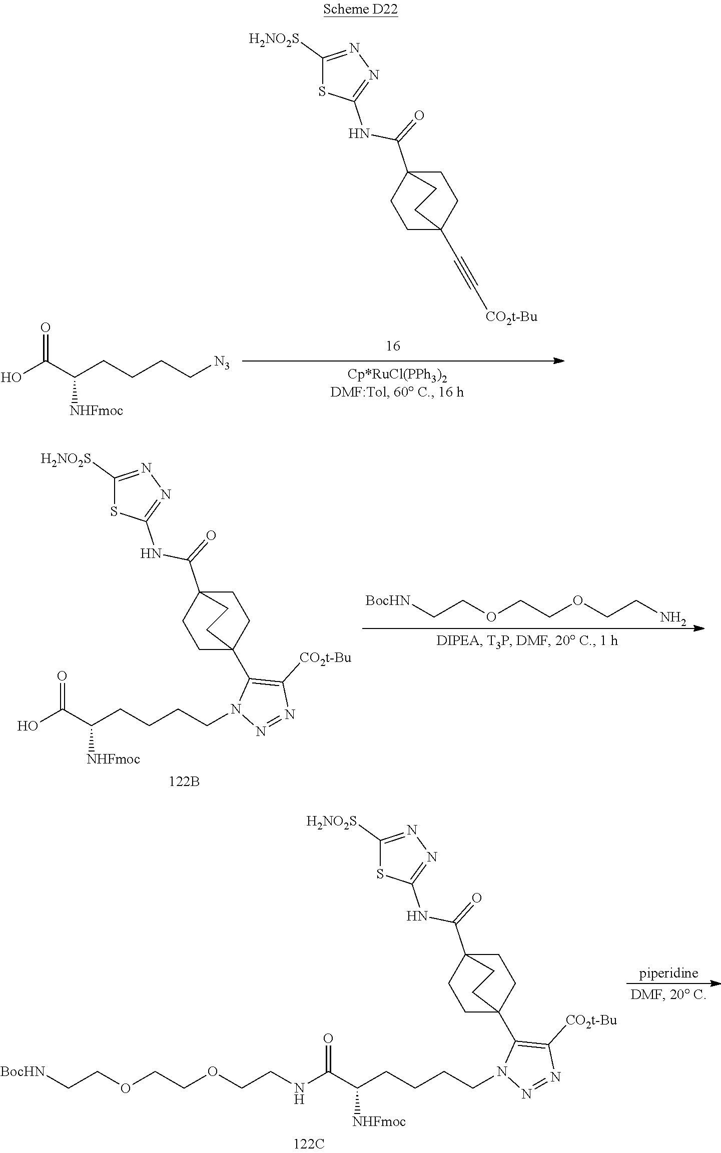

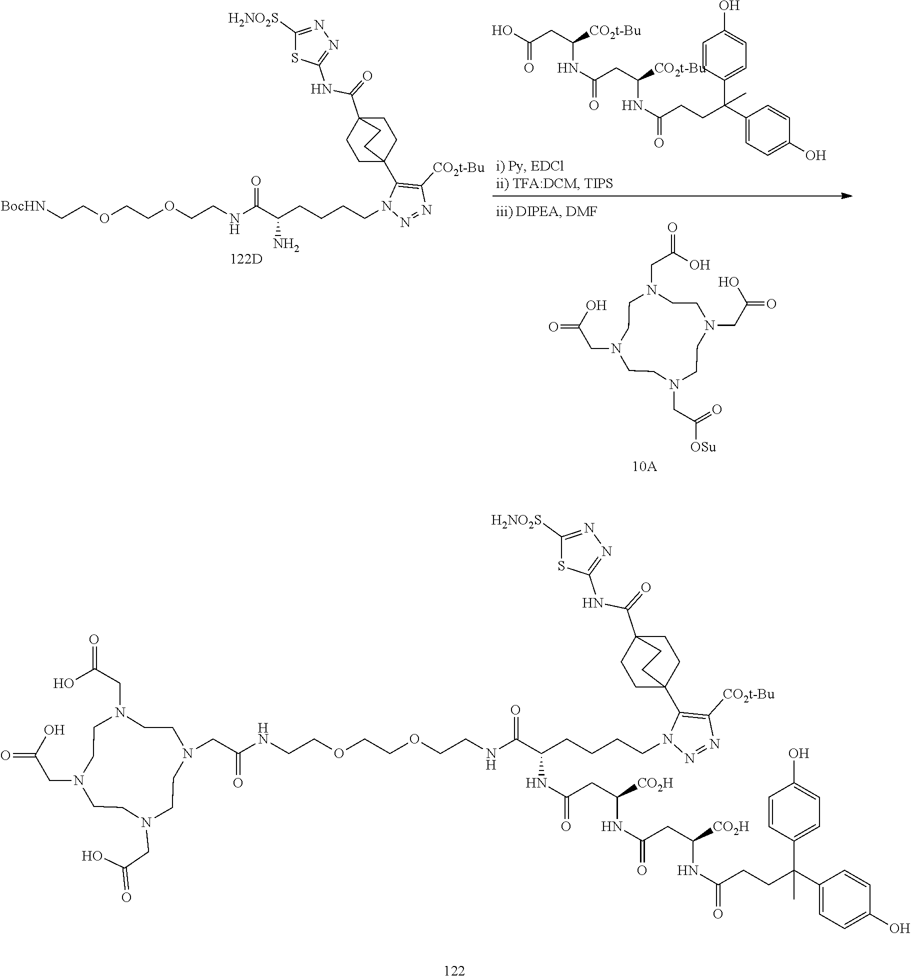

### 4.95 Preparation of Compound 122B

To a mixture of compound 16 (400 mg, 908 μmol, 1 eq) and N2-(((9H-fluoren-9-yl)methoxy)carbonyl)-N6-diazo-L-lysine (358 mg, 908 μmol, 1 eq) in DMF (12 mL) and toluene (3 mL) was added Cp*RuCl(PPh₃)₂ (217 mg, 272 μmol, 0.3 eq) at 20° C. under a nitrogen atmosphere. The mixture was stirred at 60° C. for 16 h and concentrated under reduced pressure. The residue was purified by prep-HPLC (column: Waters Xbridge BEH C18 100*30 mm*10 μm; mobile phase: [H₂O (10 mM NH₄HCO₃)-ACN]; gradient: 25%-55% B over 8.0 min) to give compound 122B (360 mg, 24% yield) as a light yellow solid; LCMS [M+H]: 835.3; ¹H NMR (400 MHz, DMSO-d₆) δ 7.88 (br d, J=7.2 Hz, 2H), 7.72 (br d, J=7.2 Hz, 2H), 7.40 (br t, J=7.0 Hz, 2H), 7.32 (br t, J=7.2 Hz, 2H), 4.47 (br t, J=7.0 Hz, 2H), 4.36-4.15 (m, 3H), 3.93 (br d, J=3.6 Hz, 1H), 2.11-1.71 (m, 16H), 1.63-1.51 (m, 9H), 1.49-1.33 (m, 3H).

### 4.96 Preparation of Compound 122C

T₃P (278 μL, 467 μmol, 50% purity, 1.5 eq) were added slowly to a solution of compound 122B (260 mg, 311 μmol, 1 eq), tert-butyl(2-(2-(2-aminoethoxy) ethoxy)ethyl) carbamate (77 mg, 311 μmol, 1 eq), DIEA (108 μL, 623 μmol, 2 eq) and DMF (6 mL). The reaction mixture was stirred at 20° C. for 1 h, quenched by addition of H₂O (10 mL) at 25° C. and the reaction mixture concentrated under reduced pressure. The residue was diluted with H₂O (20 mL) and extracted with EtOAc (80 mL). The combined organic layers were washed with H₂O (30 mL), brine (30 mL), dried (Na₂SO₄), filtered and concentrated under reduced pressure. The crude product was purified by column chromatography (SiO₂, DCM:MeOH) to give crude compound 122C (400 mg) as a light yellow solid; LCMS [M+H]: 1065.6; ¹H NMR (400 MHz, METHANOL-d4) δ 7.82-7.71 (m, 2H), 7.71-7.55 (m, 2H), 7.42-7.24 (m, 4H), 4.65-4.47 (m, 2H), 4.47-4.32 (m, 2H), 4.23 (brd, J=6.8 Hz, 1H), 4.15-4.06 (m, 1H), 3.65-3.43 (m, 9H), 3.38 (br t, J=5.2 Hz, 2H), 3.20 (br t, J=5.6 Hz, 3H), 2.25-2.08 (m, 7H), 2.06-1.81 (m, 11H), 1.59 (s, 9H), 1.41 (s, 9H).

### 4.97 Preparation of Compound 122D

To a mixture of compound 122C (400 mg, 375 μmol, 1 eq) in DMF (5 mL) was added piperidine (185 μL, 1.88 mmol, 5 eq) in one portion at 20° C. under a nitrogen atmosphere. The mixture was stirred at 20° C. for 1 h and concentrated under reduced pressure. The residue was washed with MTBE (3× 10 mL) to give crude compound 122D (360 mg) as a light yellow solid; LCMS [M+H]: 843.4; ¹H NMR (400 MHz, METHANOL-d₄) δ 4.59 (br t, J=7.4 Hz, 1H), 3.70-3.46 (m, 7H), 3.44-3.35 (m, 3H), 3.26-3.18 (m, 4H), 3.16-3.06 (m, 1H), 2.25-2.11 (m, 5H), 2.07-1.93 (m, 6H), 1.83-1.64 (m, 4H), 1.42 (s, 9H), 1.25-1.14 (m, 9H).

### 4.98 Preparation of Compound 122

Compound 122 was prepared from compound 122D as previously depicted; LCMS [M+H]: 1571.7.

### D23. Synthesis of Compound 123

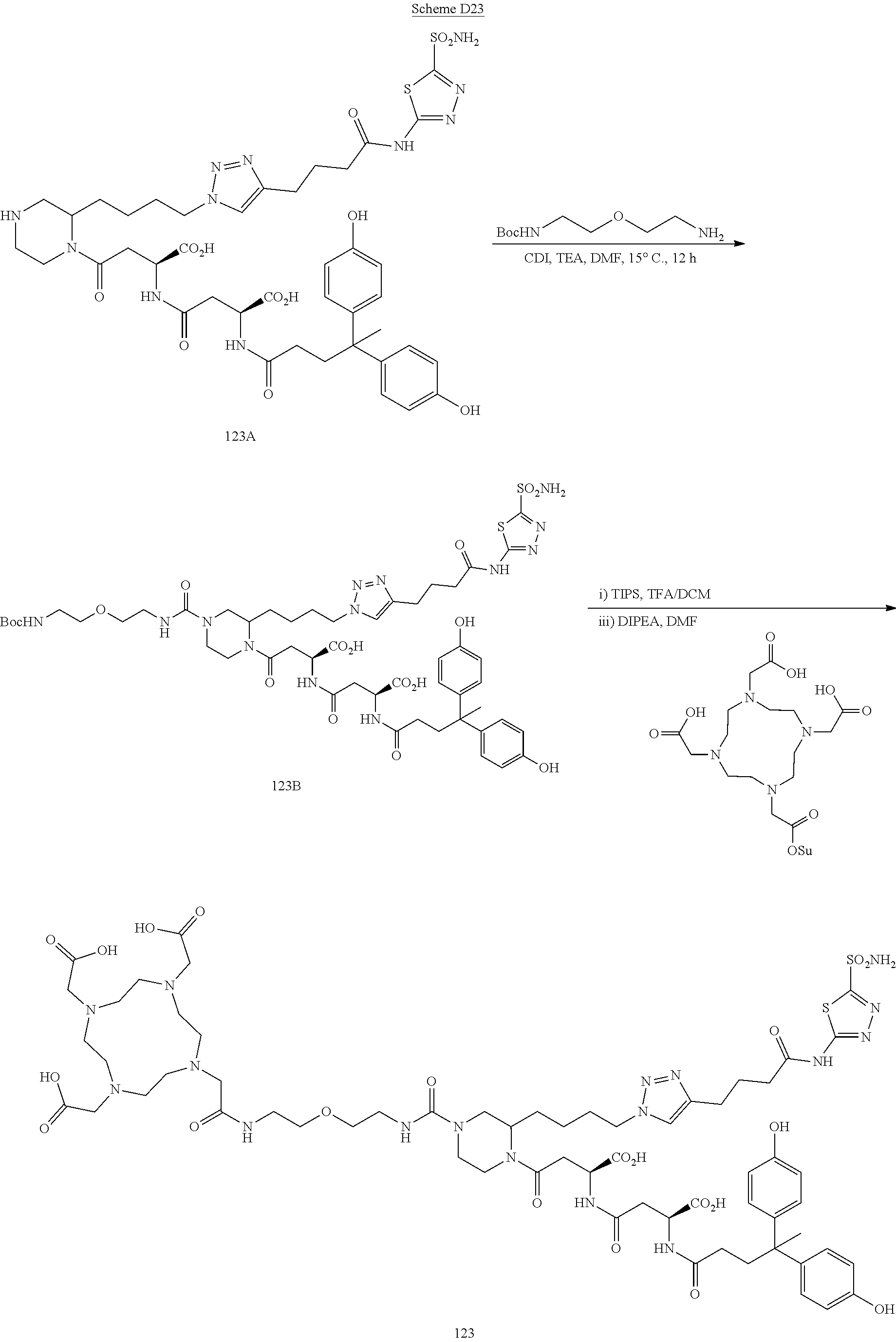

### 4.99 Synthesis of Compound 123B

To a solution of tert-butyl(2-(2-aminoethoxy)ethyl) carbamate (60 mg, 294 μmol, 1 eq) and TEA (41 μL, 294 μmol, 1 eq) in DMF (1 mL) was added CDI (48 mg, 294 μmol, 1 eq) at 15° C., and the mixture stirred at 15° C. for 1 h. A solution of compound 123A (314 mg, 294 μmol, 1 eq, TFA) and TEA (41 μL, 294 μmol, 1 eq) in DMF (1 mL) was added and the mixture stirred at 15° C. for 11 h. The reaction mixture was concentrated under reduced pressure and the residue purified by prep-TLC (SiO₂, EtOAc:MeOH) to give compound 123B (260 mg, 75% yield) as white solid; LCMS [M+H]: 1186.8; ¹H NMR (400 MHz, DMSO-d₆) δ 8.28-8.11 (m, 1H), 7.93-7.85 (m, 1H), 7.63 (s, 2H), 7.01 (s, 3H), 6.92 (d, J=8.5 Hz, 4H), 6.76 (t, J=5.3 Hz, 1H), 6.63 (d, J=8.5 Hz, 4H), 6.55 (br s, 1H), 4.50-4.33 (m, 2H), 4.31-4.10 (m, 4H), 3.94-3.75 (m, 3H), 3.71-3.57 (m, 1H), 3.17 (s, 6H), 3.05 (q, J=5.4 Hz, 3H), 2.89 (s, 1H), 2.73 (s, 2H), 2.70-2.59 (m, 4H), 2.40 (br s, 2H), 2.22-2.13 (m, 2H), 1.97-1.82 (m, 4H), 1.82-1.66 (m, 2H), 1.47-1.41 (m, 3H), 1.36 (s, 9H), 1.34-1.21 (m, 2H).

### 4.100 Synthesis of Compound 123

Compound 123 was prepared from compound 123B as previously depicted; LCMS [M+H]: 1472.3; ¹H NMR (400 MHz, DMSO-d₆) δ 12.99 (brs, 2H), 9.16 (br s, 1H), 8.54 (brs, 1H), 8.32 (s, 2H), 8.16-8.01 (m, 1H), 7.96-7.87 (m, 1H), 7.87-7.81 (m, 1H), 6.92 (d, J=8.5 Hz, 4H), 6.64 (d, J=8.5 Hz, 4H), 4.57 (d, J=5.0 Hz, 1H), 4.51-4.43 (m, 1H), 4.42-4.32 (m, 1H), 4.29-4.19 (m, 3H), 4.18-4.03 (m, 4H), 3.95-3.77 (m, 6H), 3.71-3.48 (m, 5H), 3.48-3.32 (m, 10H), 3.29-3.16 (m, 6H), 3.11 (br s, 6H), 2.86-2.53 (m, 10H), 2.21-2.12 (m, 2H), 2.00-1.88 (m, 2H), 1.88-1.81 (m, 2H), 1.81-1.70 (m, 2H), 1.56-1.34 (m, 5H), 1.29-1.06 (m, 2H).

### D24. Synthesis of Compound 124

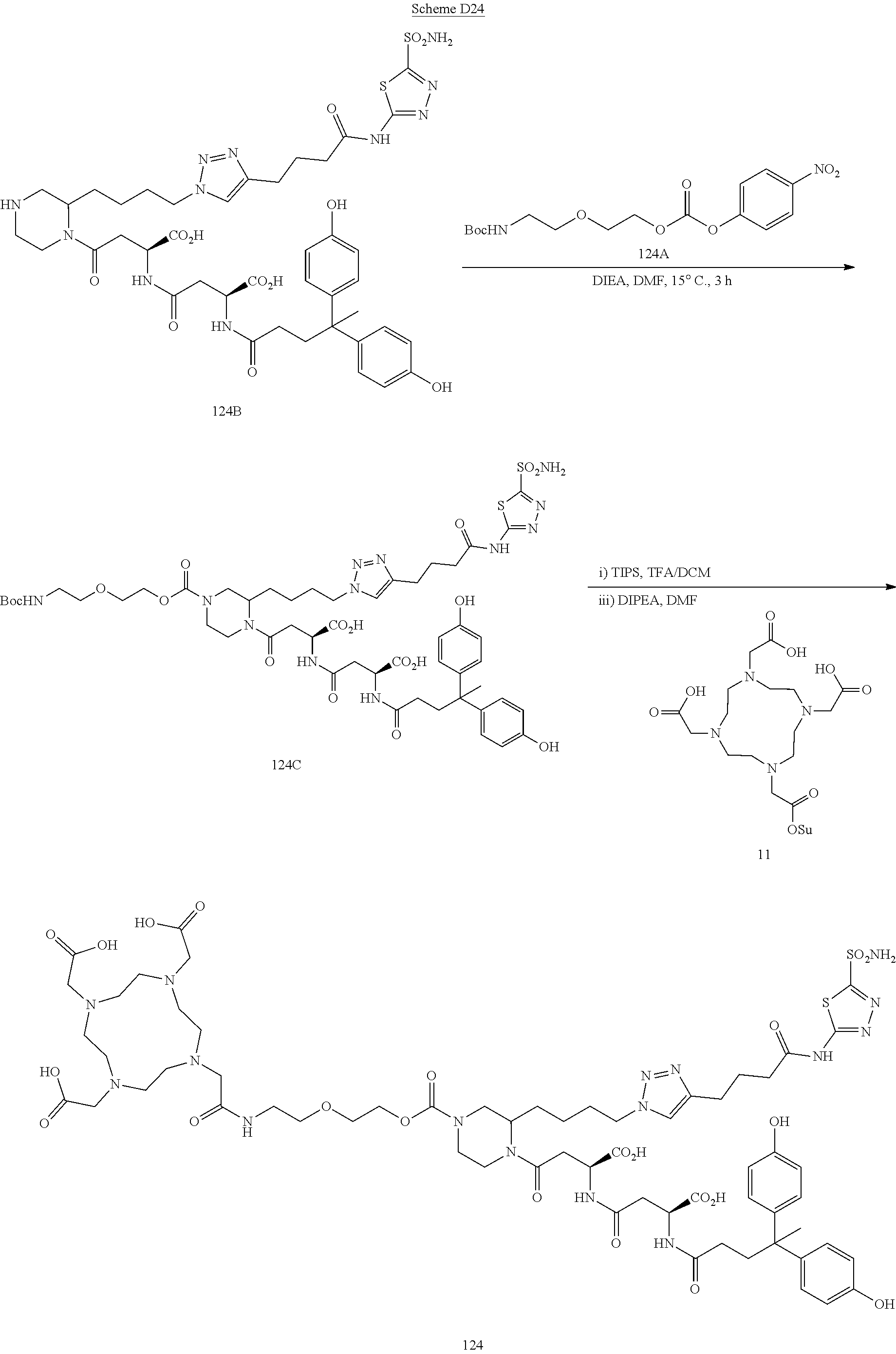

### 4.101 Synthesis of Compound 124A

To a solution of tert-butyl(2-(2-hydroxyethoxy)ethyl) carbamate (200 mg, 974 μmol, 1 eq) and TEA (271 μL, 1.95 mmol, 2 eq) in THF (5 mL) was added 4-nitrophenyl carbonochloridate (216 mg, 1.07 mmol, 1.1 eq) at 15° C., and the mixture stirred at 15° C. for 12 h. The reaction mixture was concentrated under reduced pressure, the residue diluted with H₂O (10 mL) and extracted with EtOAc (3×10 mL). The combined organic extracts were dried (Na₂SO₄), filtered and concentrated under reduced pressure. The residue was purified by prep-TLC (SiO₂, EtOAc:petroleum ether) to give compound 124A (190 mg, 53% yield) as a colorless oil; ¹H NMR (400 MHz, CHLOROFORM-d) δ 8.32-8.27 (m, 2H), 7.44-7.37 (m, 2H), 4.93 (br s, 1H), 4.48-4.42 (m, 2H), 3.82-3.74 (m, 2H), 3.60 (t, J=5.1 Hz, 2H), 3.36 (q, J=5.1 Hz, 2H), 1.46 (s, 9H).

### 4.102 Synthesis of Compound 124C

To a solution of compound 124B (350 mg, 327 μmol, TFA) and DIEA (132 μL, 756 μmol, 2 eq) in DMF (3 mL) was added compound 124A (140 mg, 378 μmol, 1 eq) at 15° C., and the mixture stirred at 15° C. for 3 h. The reaction mixture was filtered and concentrated under reduced pressure. The residue was purified by prep-TLC (SiO₂, EtOAc:MeOH) to give compound 124C (330 mg, 74% yield) as a colorless solid; LCMS [M+H]: 1187.4; ¹H NMR (400 MHz, DMSO-d₆) δ 8.29-8.01 (m, 2H), 7.96-7.83 (m, 1H), 7.70-7.56 (m, 1H), 6.92 (br d, J=8.3 Hz, 4H), 6.63 (br d, J=8.3 Hz, 4H), 4.49-4.34 (m, 2H), 4.32-4.17 (m, 4H), 4.15-4.06 (m, 2H), 3.96-3.64 (m, 4H), 3.59-3.53 (m, 2H), 3.42-3.36 (m, 2H), 3.18-3.13 (m, 7H), 3.10-3.01 (m, 4H), 2.70-2.59 (m, 4H), 2.43-2.37 (m, 2H), 2.20-2.12 (m, 2H), 1.95-1.83 (m, 4H), 1.47-1.40 (m, 3H), 1.35 (s, 9H), 1.32-1.21 (m, 2H).

### 4.103 Synthesis of Compound 124

Compound 124 was prepared from compound 124C from procedures previously depicted; LCMS [M+H]: 1473.4; ¹H NMR (400 MHz, DMSO-d₆) δ 13.13-12.91 (m, 1H), 9.15 (d, J=6.4 Hz, 2H), 8.50 (d, J=1.9 Hz, 1H), 8.32 (s, 2H), 8.19-8.02 (m, 1H), 7.92 (t, J=6.5 Hz, 1H), 7.87-7.81 (m, 1H), 6.92 (d, J=8.3 Hz, 4H), 6.64 (d, J=8.5 Hz, 4H), 4.57 (dd, J=2.3, 3.4 Hz, 1H), 4.51-4.39 (m, 2H), 4.30-4.21 (m, 2H), 4.16-4.09 (m, 2H), 3.59 (br s, 8H), 3.54 (d, J=6.4 Hz, 2H), 3.50-3.43 (m, 4H), 3.27 (d, J=5.5 Hz, 8H), 3.15-2.99 (m, 10H), 2.72-2.56 (m, 10H), 2.36-2.31 (m, 2H), 2.21-2.12 (m, 2H), 1.94 (quin, J=7.3 Hz, 2H), 1.89-1.74 (m, 4H), 1.45 (s, 5H), 1.21-1.10 (m, 2H).

### D25. Synthesis of Compound 125

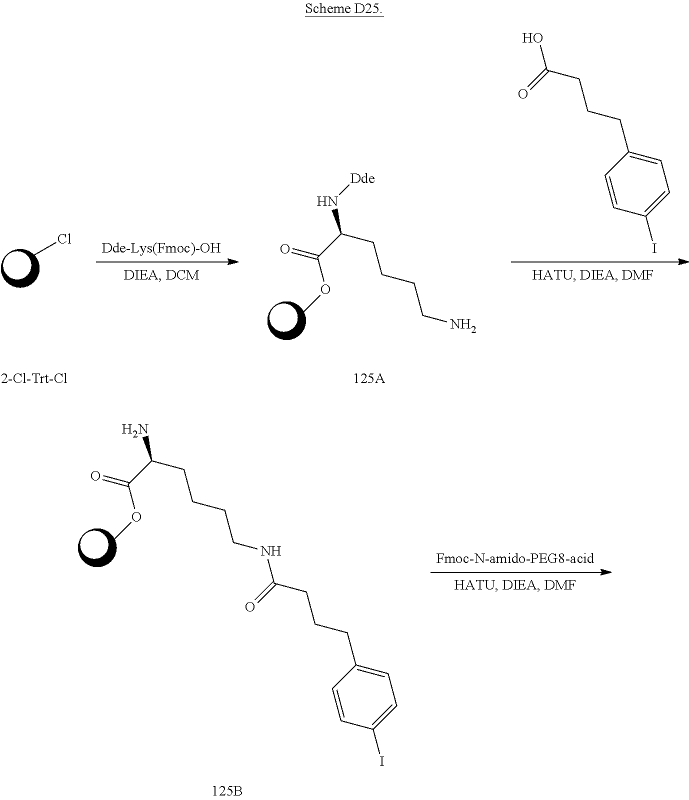

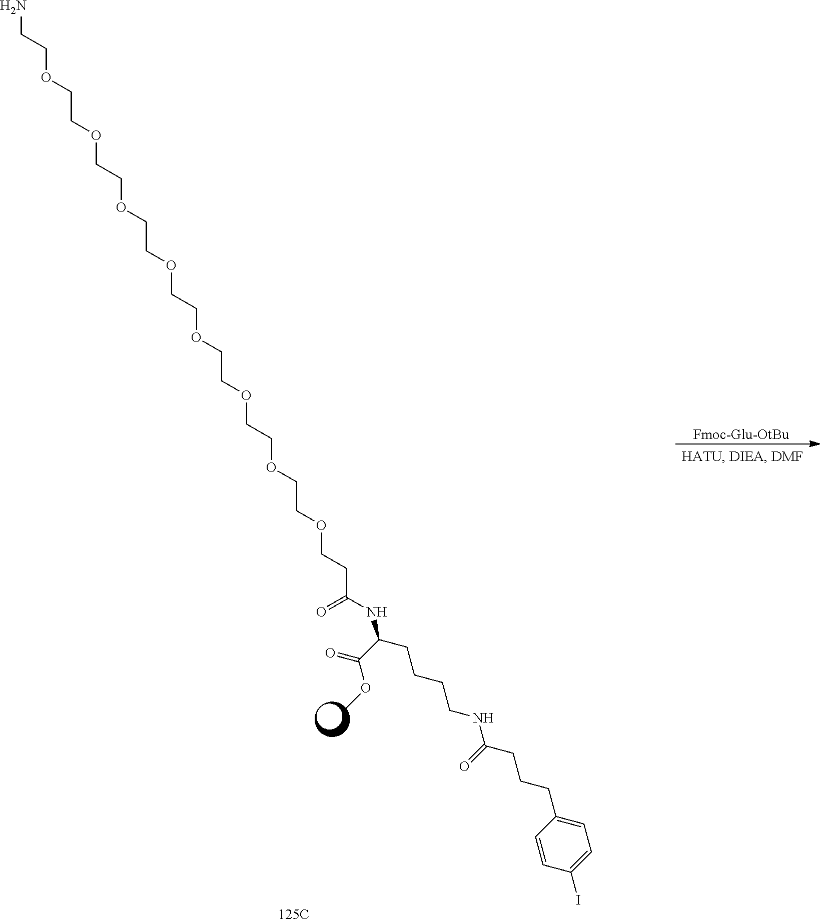 [SMILES: COC(=O)[C@H](CCCCNC(=O)CCCc1ccc(I)cc1)NC(=O)CCOCCOCCOCCOCCOCCOCCOCCOCCN ; "Fmoc-Glu-OtBu"]

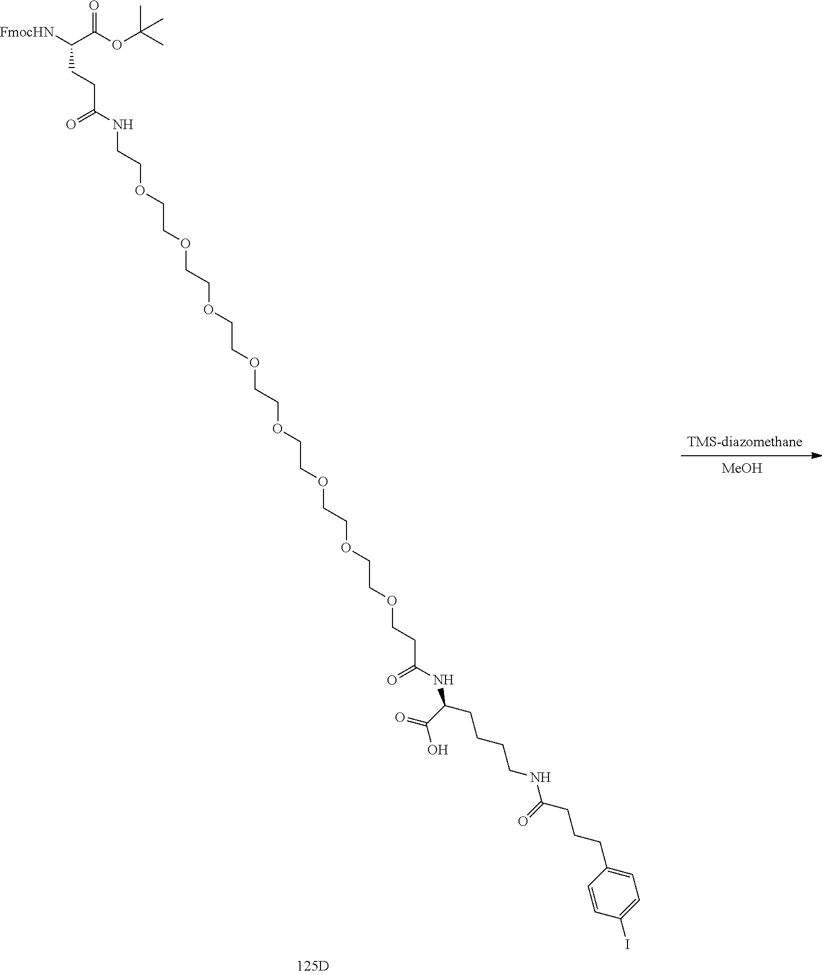

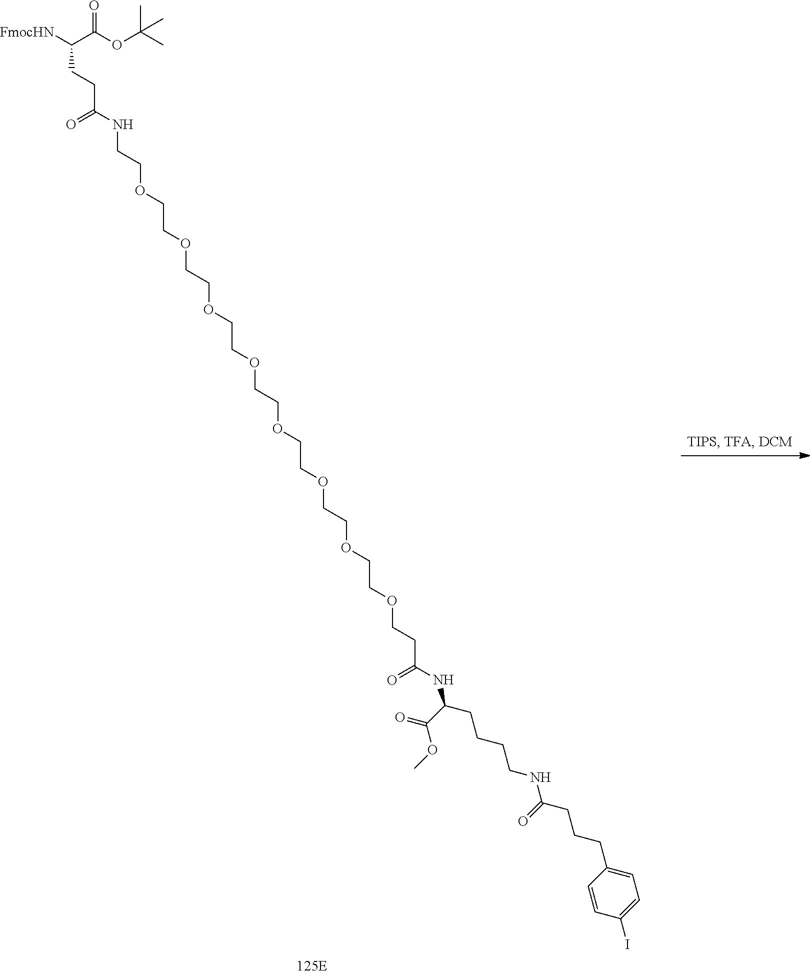

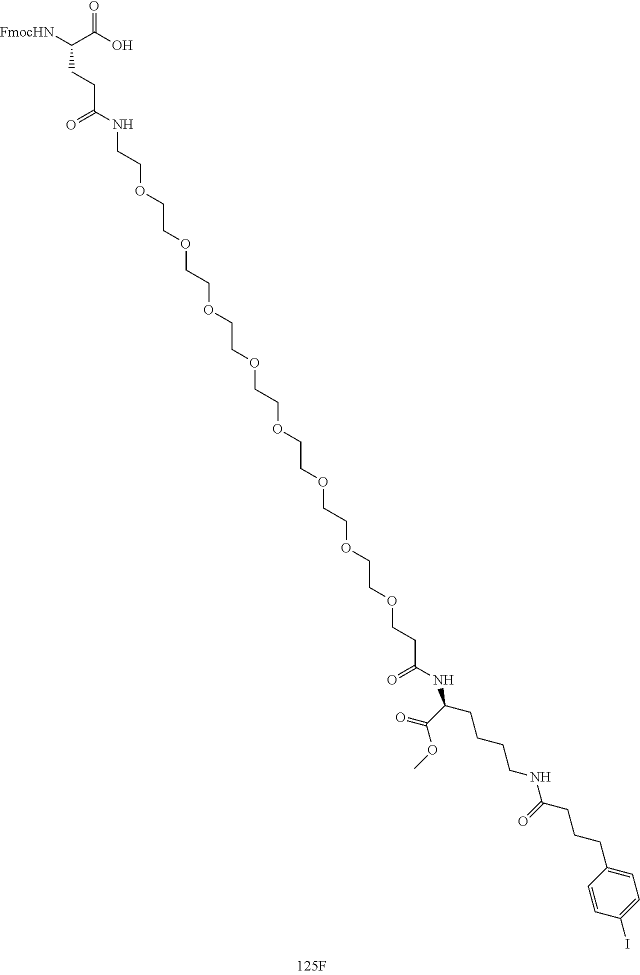

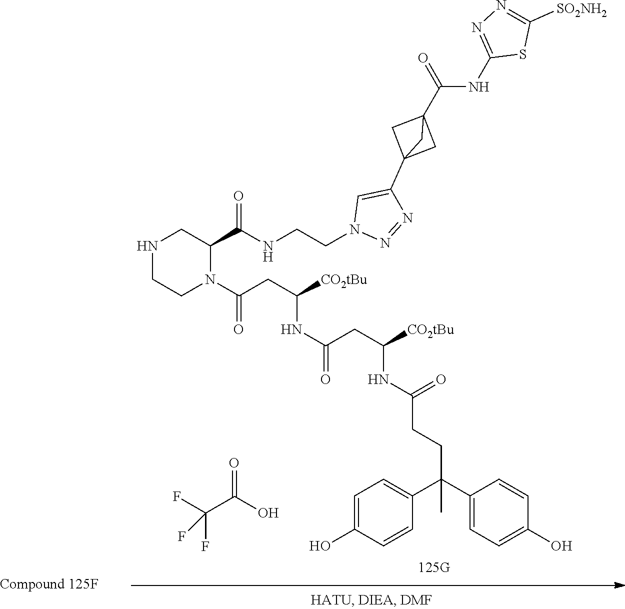 [SMILES: CC(C)(C)OC(=O)[C@H](CC(=O)N[C@@H](CC(=O)N1CCNC[C@H]1C(=O)NCCn1cc(C23CC(C(=O)Nc4nnc(S(N)(=O)=O)s4)(C2)C3)nn1)C(=O)OC(C)(C)C)NC(=O)CCC(C)(c1ccc(O)cc1)c1ccc(O)cc1 ; O=C(O)C(F)(F)F]

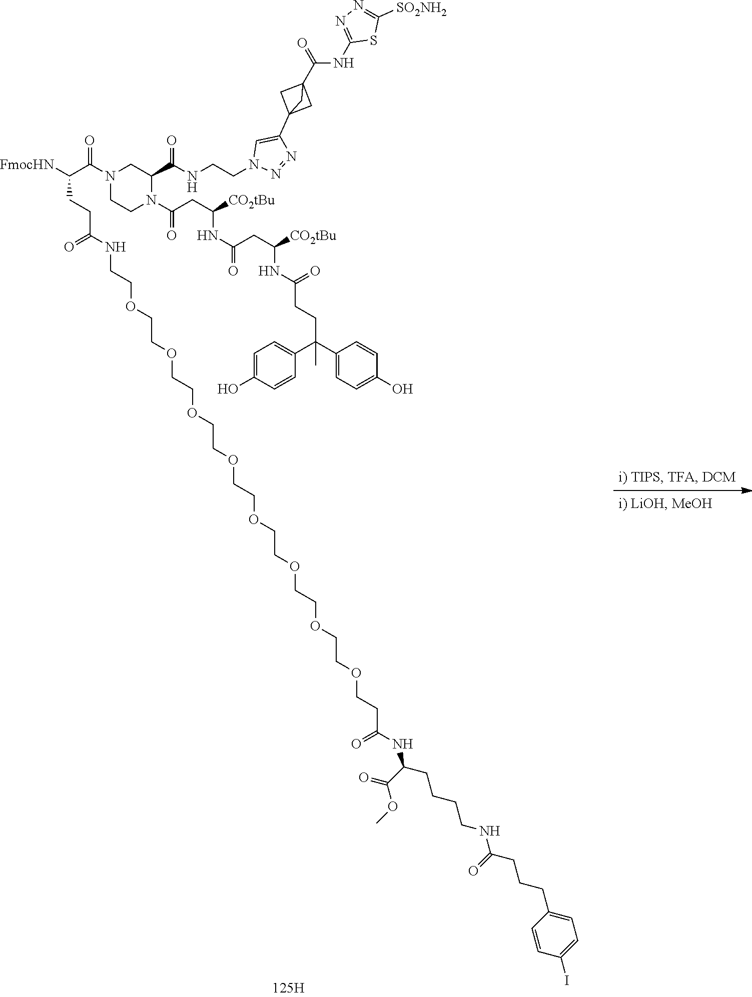

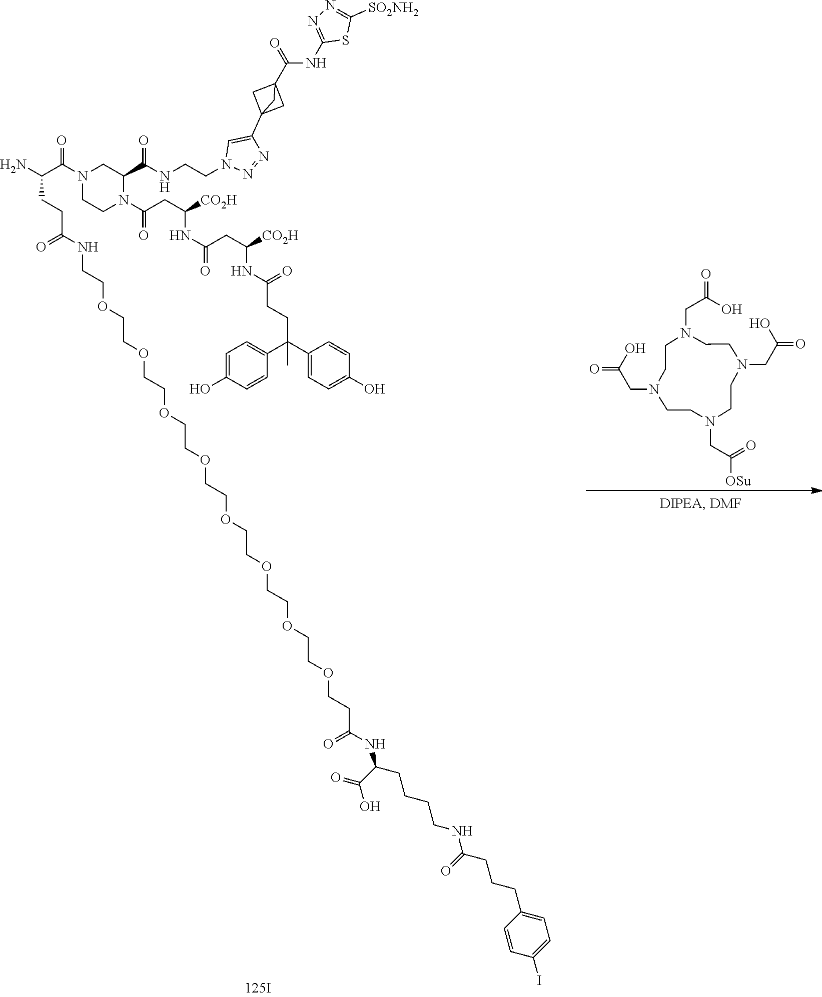

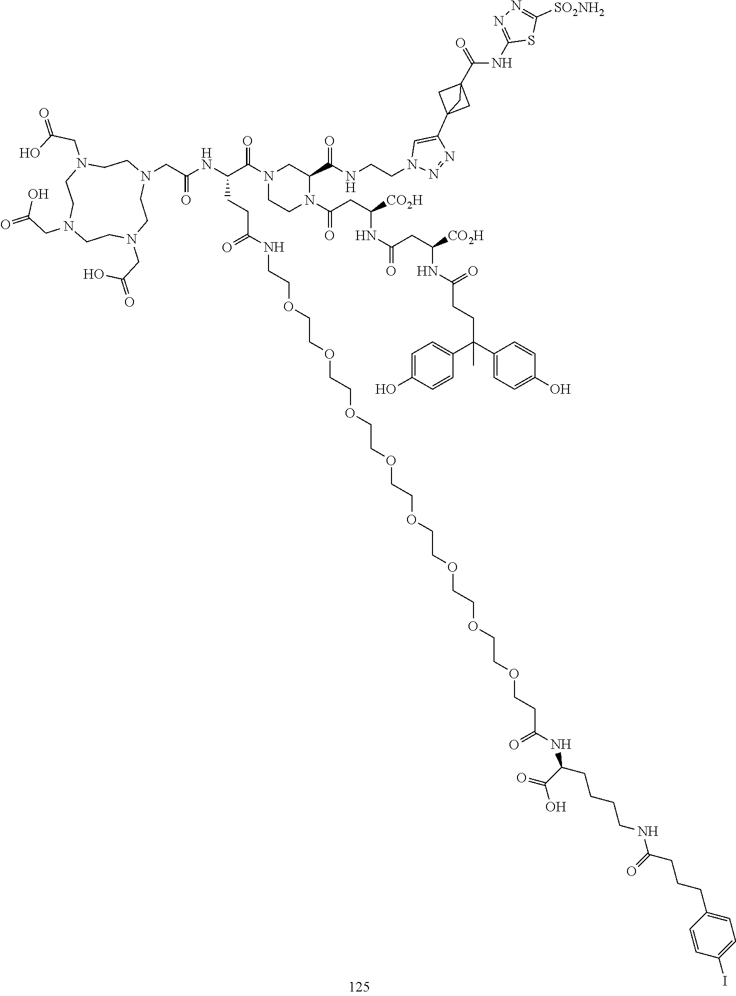 [SMILES: CC(CCC(=O)N[C@@H](CC(=O)N[C@@H](CC(=O)N1CCN(C(=O)[C@H](CCC(=O)NCCOCCOCCOCCOCCOCCOCCOCCOCCC(=O)N[C@@H](CCCCNC(=O)CCCc2ccc(I)cc2)C(=O)O)NC(=O)CN2CCN(CC(=O)O)CCN(CC(=O)O)CCN(CC(=O)O)CC2)C[C@H]1C(=O)NCCn1cc(C23CC(C(=O)Nc4nnc(S(N)(=O)=O)s4)(C2)C3)nn1)C(=O)O)C(=O)O)(c1ccc(O)cc1)c1ccc(O)cc1]

### 4.104 Preparation of Compound 125A

2-Cl-Trt-Cl resin (6.8 g, 1.0 mmol/g, 6.8 mmol, 1.7 eq) was added to a mixture of Dde-Lys (Fmoc)-OH (2.1 g, 4 mmol, 1 eq), DIEA (2.8 mL, 16 mmol, 4 eq), and DCM (3 mL) and agitated for 2 h. MeOH (3 mL) was added, the reaction mixture agitated for 30 min, filtered and the filter cake washed with 17:2:1 DCM:MeOH:DIEA (3×50 mL), DCM (3×80 mL), and DMF (2×80 mL). The reaction mixture was agitated with 20% piperidine in DMF (70 mL). The mixture was filtered, and the process repeated 5 times. The filter cake was washed with DCM (3×70 mL) and DMF (2×70 mL) to give compound 125A.

### 4.105 Preparation of Compound 125B

A solution of 4-(4-iodophenyl) butanoic acid (3.5 g, 12 mmol, 3 eq), HATU (3.0 g, 8 mmol, 2 eq), DIEA (6.3 mL, 36 mmol, 9 eq), and DMF (70 mL) was added to compound 125A (4 mmol). The mixture was agitated for 62 h and the reaction solution removed via filtration. The filter cake was washed with DMF (70 mL), DCM (3×70 mL), and DMF (2×70 mL). The reaction mixture was agitated with 2% hydrazine in DMF (75 mL). The mixture was filtered, and the process repeated 3 times. The filter cake was washed with DMF (70 mL), DCM (2×70 mL) and DMF (2×70 mL) to give compound 125B.

### 4.106 Preparation of Compound 125C

A solution of Fmoc-N-amido-PEG8-acid (5.0 g, 7.5 mmol, 1.9 eq), HATU (2.9 g, 7.6 mmol, 1.9 eq), DIEA (6.3 mL, 36 mmol, 9 eq) and DMF (70 mL) was added to compound 125B (4 mmol). The mixture was agitated for 2 h and the reaction solution removed via filtration. The filter cake was washed with DMF (70 mL), DCM (3×70 mL), and DMF (2×70 mL). The reaction was agitated with 20% piperidine in DMF (70 mL). The mixture was filtered, and this process was repeated 5 times. The filter cake was washed with DCM (2×70 mL) and DMF (2×70 mL) to give compound 125C.

### 4.107 Preparation of Compound 125D

A solution of Fmoc-Glu-OtBu (5.1 g, 12 mmol, 3 eq), HATU (3.0 g, 8 mmol, 2 eq), DIEA (6.3 mL, 36 mmol, 9 eq), and DMF (70 mL) was added to compound 125C (4 mmol). The mixture was agitated for 2 h and the reaction solution removed via filtration. The filter cake was washed with DMF (70 mL) and DCM (3×70 mL). The resin was cleaved via 5 min agitation with 20% HFP in DCM (70 mL). The mixture was filtered and this process was repeated 4 times. The filter cake was washed with DCM (70 mL). The filtrate were combined, concentrated under reduced pressure and purified by flash chromatography (EtOAc:DCM then MeOH:DCM) to give compound 125D (2.6 g, 52%) as a yellow solid; LCMS [M+H]: 1249.4; ¹H NMR (400 MHz, DMSO) δ 12.54 (s, 1H), 8.09 (d, J=7.7 Hz, 1H), 7.90 (d, J=7.4 Hz, 3H), 7.73 (h, J=6.7 Hz, 4H), 7.65-7.58 (m, 2H), 7.43 (t, J=7.5 Hz, 2H), 7.34 (t, J=7.5 Hz, 2H), 7.01 (d, J=7.8 Hz, 2H), 4.27 (dt, J=21.6, 6.6 Hz, 3H), 4.19-4.09 (m, 2H), 3.89 (t, J=7.3 Hz, 1H), 3.58 (t, J=6.7 Hz, 2H), 3.47 (s, 26H), 3.40 (t, J=6.2 Hz, 2H), 3.23-3.13 (m, 5H), 3.00 (q, J=6.7 Hz, 2H), 2.48 (s, 1H), 2.36 (p, J=7.6 Hz, 2H), 2.17 (t, J=7.7 Hz, 2H), 2.04 (t, J=7.4 Hz, 2H), 1.93 (d, J=8.3 Hz, 1H), 1.76 (q, J=7.6 Hz, 3H), 1.68 (d, J=8.0 Hz, 1H), 1.60-1.49 (m, 1H), 1.39 (s, 10H), 1.28 (d, J=7.8 Hz, 2H).

### 4.108 Preparation of Compound 125E

TMS-diazomethane (10.5 mL, 10% in hexane) was added to a solution of compound 125D (2.6 g, 2.1 mmol, 1 eq) in MeOH (10 mL) and stirred at room temperature for 5 min. TMS-diazomethane (8.0 mL, 10% in Hexane) was then added, the mixture stirred at room temperature for 5 min and concentrated under reduced pressure. The residue was purified by silica gel chromatography (MeOH:DCM) to give compound 125E (2.2 g, 85%) as a yellow solid; LCMS [(M−(Bu)+H]: 1207.3; ¹H NMR (400 MHz, DMSO) δ 8.23 (d, J=7.5 Hz, 1H), 7.90 (d, J=7.4 Hz, 3H), 7.72 (dq, J=13.9, 8.0 Hz, 4H), 7.65-7.60 (m, 2H), 7.43 (t, J=7.5 Hz, 2H), 7.34 (t, J=7.4 Hz, 2H), 7.01 (d, J=7.7 Hz, 2H), 4.35-4.09 (m, 5H), 3.88 (d, J=6.5 Hz, 1H), 3.62-3.55 (m, 5H), 3.47 (s, 27H), 3.40 (t, J=6.2 Hz, 2H), 3.23-3.16 (m, 3H), 3.00 (q, J=6.5 Hz, 2H), 2.48 (s, 1H), 2.37 (q, J=7.0 Hz, 2H), 2.17 (t, J=7.7 Hz, 2H), 2.04 (t, J=7.4 Hz, 2H), 1.93 (d, J=8.1 Hz, 1H), 1.76 (q, J=7.6 Hz, 3H), 1.65 (t, J=7.1 Hz, 1H), 1.61-1.52 (m, 1H), 1.39 (d, J=1.8 Hz, 11H), 1.27 (s, 2H).

### 4.109 Preparation of Compound 125F

Compound 125E (2.2 g, 1.8 mmol, 1 eq) was dissolved in a solution of trifluoroacetic acid (2 mL), tris(propan-2-yl)silane (2 mL), and DCM (6 mL). The mixture was stirred at room temperature overnight, concentrated, and then purified by prep-HPLC (40-65% CH₃CN in water with 0.1% FA). The material was stirred with MP-Carbonate in THF and MeOH overnight. The mixture was filtered and the filtrate concentrate to give Compound 125F (1.2 g, 56%) as a white powder; LCMS [M+H]: 1207.4; ¹H NMR (400 MHz, DMSO) δ 12.64 (s, 1H), 8.23 (d, J=7.3 Hz, 1H), 7.90 (d, J=7.4 Hz, 3H), 7.79-7.70 (m, 3H), 7.68 (d, J=8.0 Hz, 1H), 7.63 (dd, J=8.3, 2.3 Hz, 2H), 7.43 (t, J=7.4 Hz, 2H), 7.34 (t, J=7.5 Hz, 2H), 7.05-6.96 (m, 2H), 4.30-4.17 (m, 4H), 3.99-3.88 (m, 1H), 3.64-3.54 (m, 5H), 3.54-3.43 (m, 27H), 3.41-3.37 (m, 3H), 3.19 (d, J=6.0 Hz, 2H), 3.00 (d, J=6.6 Hz, 2H), 2.48 (s, 2H), 2.36 (p, J=7.5 Hz, 2H), 2.18 (t, J=7.7 Hz, 2H), 2.03 (t, J=7.4 Hz, 2H), 1.97 (s, 1H), 1.76 (q, J=7.3 Hz, 3H), 1.69-1.51 (m, 2H), 1.35 (d, J=7.4 Hz, 2H), 1.27 (s, 2H).

### 4.110 Preparation of Compound 125H

Compound 125G (56 mg, 42.6 μmol, 1 eq) was added to a solution of Compound 125F (59.6 μL, 1.4 eq), HATU (21 mg, 55.4 μmol, 1.3 eq), DIEA (23 μL, 132 μmol, 3.1 eq), and DMF (1 mL). The mixture was stirred at room temperature for 1 h and purified by prep-HPLC (40-70% CH₃CN in water with 0.1% TFA) to give compound 125H (88 mg, 90%) as a white foam; LCMS [M/2+H]: 1148.8.

### 4.111 Preparation of Compound 125I

Compound 125H (88 mg, 38.3 μmol, 1 eq) was dissolved in a solution of trifluoroacetic acid (0.4 mL), tris(propan-2-yl) silane (0.4 mL), and DCM (1.2 mL). The mixture was stirred at room temperature overnight and concentrated under reduced pressure. The crude material was diluted with THF (1.5 mL), MeOH (0.75 mL), and a 2.0 M LiOH solution (0.2 mL), the mixture heated at 50° C. for 1 h, concentrated under reduced pressure, and purified by prep-HPLC (30-50% CH₃CN in water with 0.1% TFA) to give compound 125I (48 mg, 64%) as a white powder; LCMS [M/2+H]: 974.7.

### 4.112 Preparation of Compound 125

Compound 125 was prepared from compound 125 from procedures previously depicted; LCMS [M/2+H]: 1167.7.

Compounds in table 9.18 were prepared by procedures depicted in Scheme D24.

TABLE 9

| Compound | m/z |
|---|---|
| 125.1 | [M/2 + H]: 1121.8 |

### D26. Synthesis of Compound 126

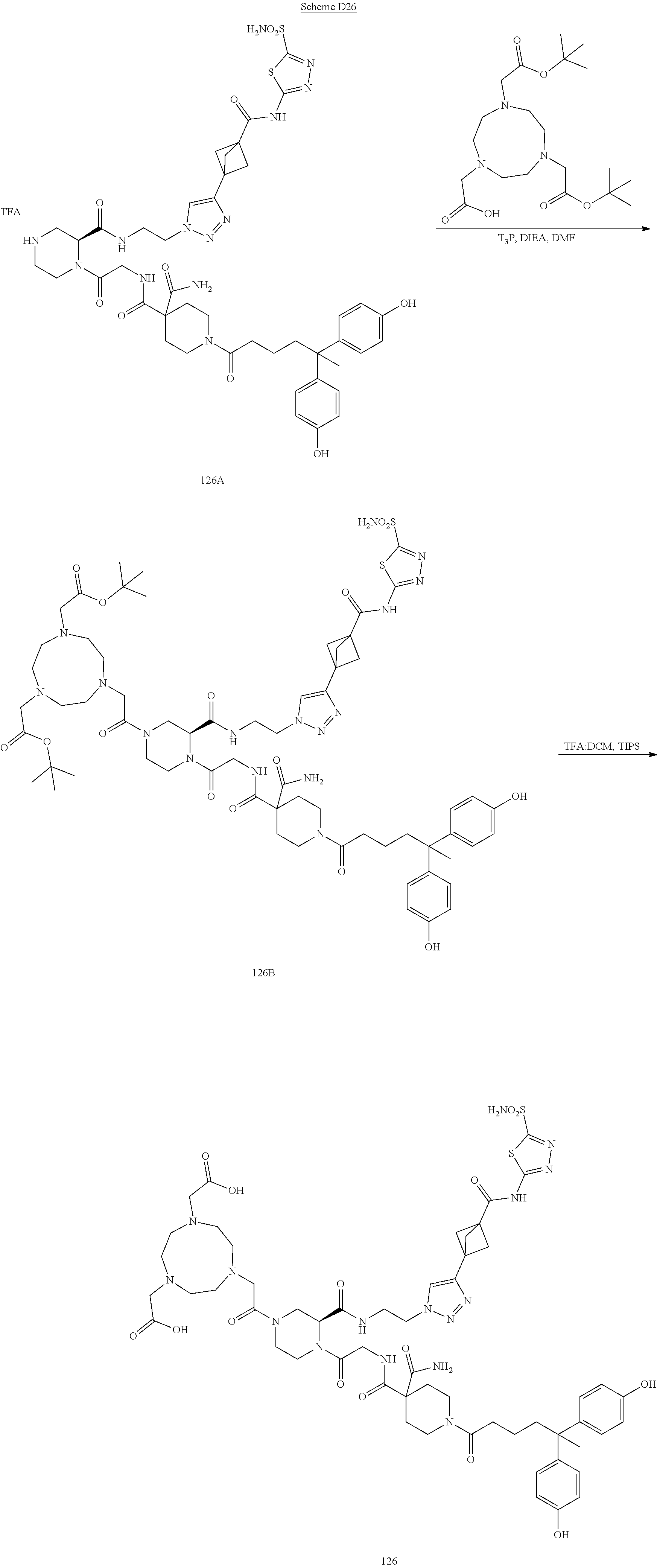

### 4.113 Preparation of Compound 126B

Propylphosphonic anhydride (50% in DMF, 270 μL, 906 μmol, 2 eq) was added to a solution of compound 126A (500 mg, 453 μmol, 1.0 eq), 2-(4,7-bis(2-(tert-butoxy)-2-oxoethyl)-1,4,7-triazonan-1-yl)acetic acid (245 mg, 589 μmol, 1.3 eq), DIEA (316 μL, 1.81 mmol, 4 eq), and DMF (2.5 mL) at 0° C. The reaction was stirred at room temperature for 15 min and purified directly by prep-HPLC (30-50% CH₃CN in water with 0.1% TFA) to give compound 126B (472 mg, 75%) as a white foam; LCMS [M/2+H]: 694.5; ¹H NMR (400 MHz, DMSO) δ 13.23 (s, 1H), 9.91 (s, 1H), 8.37 (s, 3H), 8.06 (d, J=2.6 Hz, 1H), 8.00-7.79 (m, 2H), 7.48-7.26 (m, 2H), 6.93 (d, J=8.6 Hz, 4H), 6.63 (d, J=8.4 Hz, 4H), 4.96-4.71 (m, 1H), 4.50-3.69 (m, 9H), 3.69-3.36 (m, 9H), 3.25 (d, J=12.2 Hz, 10H), 2.97-2.64 (m, 7H), 2.44 (d, J=4.6 Hz, 6H), 2.24 (d, J=7.5 Hz, 2H), 2.05-1.76 (m, 6H), 1.54-1.30 (m, 20H), 1.27-1.18 (m, 2H).

### 4.114 Preparation of Compound 126

Compound 126B (468 mg) was dissolved in trifluoroacetic acid (0.9 mL), tris(propan-2-yl)silane (0.9 mL), and DCM (2.7 mL) was added and was stirred at 40° C. for 4 h. The mixture was concentrated, basified with an NH₄OH solution, and purified by prep-HPLC (10-45% CH₃CN in water with 0.5% AcOH) to give Compound 126 (231 mg, 54%); LCMS [M/2+H]: 638.4; ¹H NMR (400 MHz, DMSO) δ 11.95 (s, 1H), 9.22 (s, 2H), 8.42-8.09 (m, 3H), 8.03-7.83 (m, 2H), 7.46 (s, 1H), 7.29 (m, 1H), 6.92 (d, J=8.6 Hz, 4H), 6.63 (d, J=8.4 Hz, 4H), 4.62 (m, 1H), 4.47-4.12 (m, 4H), 3.97 (m, 1H), 3.68 (m, 4H), 3.52 (s, 6H), 3.19 (s, 3H), 3.05 (s, 4H), 2.91 (m, 9H), 2.42 (m, 8H), 2.24 (s, 3H), 1.94 (s, 3H), 1.78 (m, 2H), 1.47 (s, 3H), 1.23 (m, 2H).

Compounds in table 9.19 were prepared by procedures depicted in Scheme D25.

TABLE 9

| Compound | m/z |
|---|---|
| 126.1 | [M/2 + H]: 638.3 |
| 126.2 | [M/2 + H]: 579.2 |
| 126.3 | [M/2 + H]: 646.8 |
| 126.4 | [M/2 + H]: 640.8 |
| 126.5 | [M/2 + H]: 574.8 |

### D27. Synthesis of Compound 127

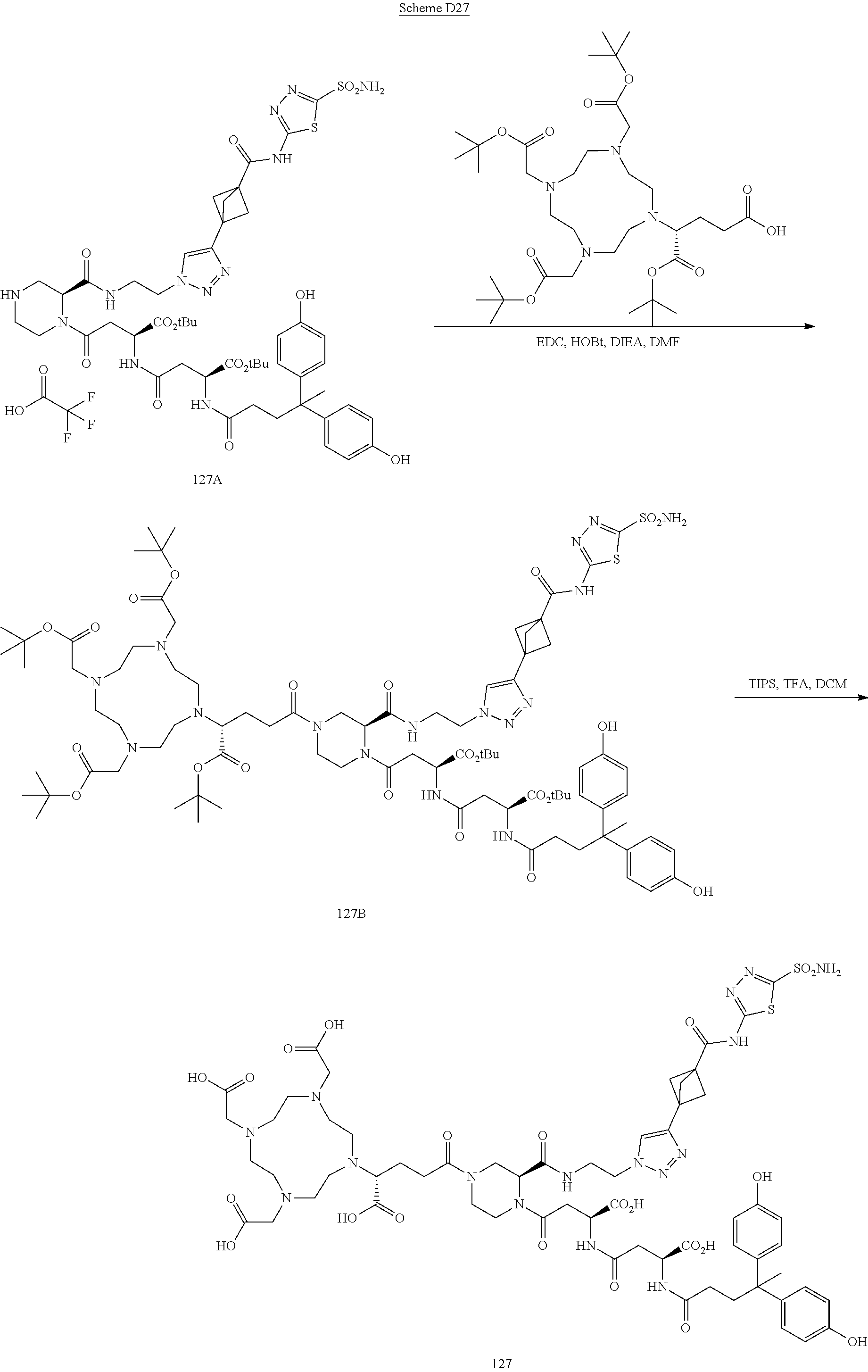

4.115 Preparation of compound 127B

Compound 127A (75 mg, 61.4 μmol, 1 eq) was added to a solution of (R)-5-(tert-butoxy)-5-oxo-4-(4,7,10-tris(2-(tert-butoxy)-2-oxoethyl)-1,4,7,10-tetraazacyclododecan-1-yl)pentanoic acid (60 mg, 85.6 μmol, 1.4 eq), EDCl (30 mg, 156 μmol, 2.6 eq), HOBt (15 mg, 111 μmol, 1.8 eq), DIEA (70 μL, 402 μmol, 6.5 eq), and DMF (1.0 mL) at 0° C. The mixture was warmed to room temperature over 2 h and purified by prep-HPLC (20-75% CH₃CN in water with 0.1% TFA) to give compound 127B (58 mg, 53%) as a white foam; LCMS [M/2+H]: 895.9.

### 4.116 Preparation of Compound 127

Compound 127B (58 mg, 32 μmol, 1 eq) was dissolved in a solution of trifluoroacetic acid (0.4 mL), tris(propan-2-yl)silane (0.4 mL), and DCM (1.2 mL). The mixture was stirred at room temperature overnight, concentrated under reduced pressure and purified by prep-HPLC (10-30% CH₃CN in water with 0.1% TFA) to Compound 127 (7.0 mg, 14%); LCMS [M/2+H]: 727.4.

Compounds in table 9.20 were prepared by procedures depicted in Scheme D26.

TABLE 9

| Compound | m/z |
|---|---|
| 127.1 | [M/2 + H]: 724.8 |
| 127.2 | [M/2 + H]: 724.8 |
| 127.3 | [M/2 + H]: 674.3 |
| 127.4 | [M/2 + H]: 732.0 |
| 127.5 | [M/2 + H]: 730.1 |
| 127.6 | [M/2 + H]: 751.2 |
| 127.7 | [M/2 + H]: 744.1 |
| 127.8 | [M/2 + H]: 743.0 |
| 127.9 | [M/2 + H]: 717.0 |

### D28. Synthesis of Compound 128

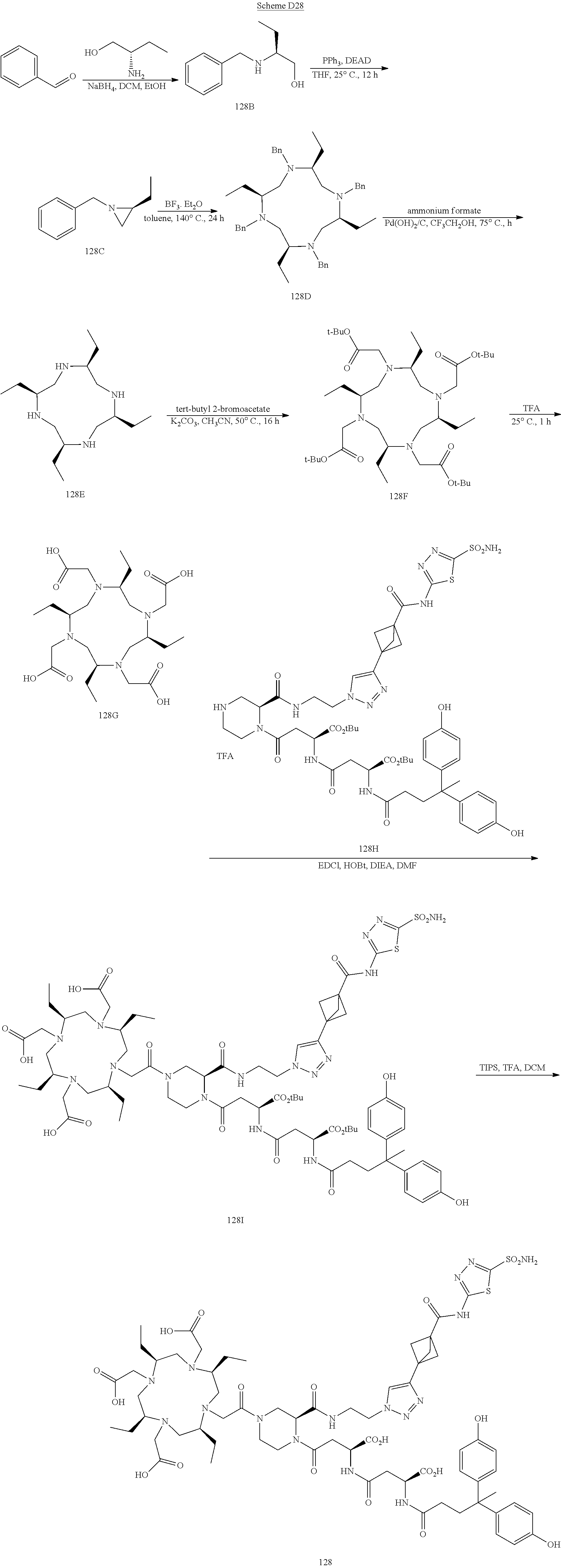

### 4.117 Preparation of Compound 128B

To a solution of (2S)-2-aminobutan-1-ol (89.4 mL, 942 mmol, 1 eq) in DCM (1000 mL) was added benzaldehyde (95 mL, 942 mmol, 1 eq) at 0° C., and the mixture stirred at 25° C. for 2 h. Na₂SO₄ was added to the mixture, the reaction filtered and the filtrate concentrated under reduced pressure. The residue was taken up in EtOH (1600 mL), NaBH₄ (40.8 g, 1.08 mol, 1.14 eq) added portion wise at 0° C. and the mixture stirred at 25° C. for 2 h. The reaction was added to a saturated aqueous solution of NH₄Cl (1000 mL) at 0° C., the residue stirred at 0° C. for 1 h and concentrated under reduced pressure. The mixture was basified with 1N NaOH to pH=9, the aqueous phase extracted with EtOAc (3×1000 mL). The combined organic layers were washed with brine (2000 mL), dried (Na₂SO₄), filtered, and concentrated under reduced pressure. The crude was suspended in EtOAc (500 mL), stirred at 20° C. for 20 min, filtered, the cake washed with EtOAc (2×300 mL) and dried under reduced pressure to give compound 128B (125 g, 68% yield) as a white solid; ¹H NMR (400 MHz, CDCl₃) δ 7.36-7.32 (m, 4H), 7.30-7.23 (m, 1H), 3.88-3.75 (m, 2H), 3.67 (dd, J=4.0, 10.6 Hz, 1H), 3.34 (dd, J=6.4, 10.8 Hz, 1H), 2.69-2.59 (m, 1H), 2.39-1.83 (m, 2H), 1.66-1.35 (m, 2H), 0.94 (t, J=7.6 Hz, 3H).

### 4.118 Preparation of Compound 128C

To a solution of compound 128B (60 g, 335 mmol, 1 eq) in THF (600 mL) was added PPh₃ (123 g, 469 mmol, 1.4 eq) and DEAD (85.2 mL, 469 mmol, 1.4 eq) at 0° C., and the mixture stirred at 25° C. for 12 h under a nitrogen atmosphere. Water (1000 mL) was added, the residue adjusted to pH=3 with 1M HCl at 0° C., extracted with EtOAc (3×500 mL), the pH of the aqueous phase adjusted to pH=8 by adding ammonia water at 0° C., and extracted with DCM (3×500 mL). The combined organics were dried (Na₂SO₄), filtered and concentrated under reduced pressure to give compound 128C (53 g, 98% yield) as a white solid; ¹H NMR (400 MHz, CDCl₃) δ 7.27 (s, 4H), 7.21-7.15 (m, 1H), 3.50-3.18 (m, 2H), 1.55 (d, J=2.8 Hz, 1H), 1.40-1.28 (m, 4H), 0.81 (t, J=7.4 Hz, 3H).

### 4.119 Preparation of Compound 128D

To a solution of compound 128C (30 g, 186 mmol, 1 eq) in toluene (420 mL) was added BF₃. Et₂O (155 μL, 3.72 mmol, 0.02 eq), and the mixture stirred under reflux at 140° C. for 24 h. The reaction was cooled, saturated sodium bicarbonate (300 mL) added, the reaction concentrated under reduced pressure and the residue purified by recrystallization in EtOAc (300 mL). The solid was washed with water (300 mL) to give crude compound 128D (8.1 g) as a colorless oil.

### 4.120 Preparation of Compound 128E

To a solution of compound 128D (8.0 g, 12.4 mmol, 1 eq) in CF₃CH₂OH (240 mL) was added Pd(OH)₂/C (5.0 g, 20% purity) and ammonium formate (10.2 g, 161 mmol, 13 eq). The reaction was heated at 75° C. for 24 h under a nitrogen atmosphere, filtered and the filtrate concentrated under reduced pressure to give crude compound 128E (4.3 g) as a colorless oil; ¹H NMR (400 MHz, CDCl₃) δ 2.75 (dd, J=2.0, 13.0 Hz, 4H), 2.47-2.30 (m, 8H), 1.64-1.49 (m, 4H), 1.25-1.14 (m, 4H), 0.87 (t, J=7.6 Hz, 12H).

### 4.121 Preparation of Compound 128F

To a solution of compound 128E (8.5 g, 29.9 mmol, 1 eq) in CH₃CN (85 mL) was added K₂CO₃ (28.9 g, 209 mmol, 7 eq) and tert-butyl 2-bromoacetate (26.5 mL, 179 mmol, 6 eq) at 0° C., and the mixture stirred at 50° C. for 16 h under a nitrogen atmosphere. The mixture was cooled to room temperature and filtered, the filtrate concentrated, the residue dissolved in 2M HCl (600 mL) and extracted with EtOAc (300 mL). The pH of the aqueous phase was adjusted to pH=8 by adding ammonia water and the aqueous layer extracted with dichloromethane (2×300 ml) to give compound 128F (9.72 g, 44% yield) as a yellow solid; ¹H NMR (400 MHz, CDCl₃) δ 3.28-3.17 (m, 4H), 3.04-2.74 (m, 10H), 2.71-2.55 (m, 3H), 2.18-2.07 (m, 5H), 1.43-1.39 (m, 36H), 1.35-1.25 (m, 3H), 0.96-0.84 (m, 16H).

### 4.122 Preparation of Compound 128G

To a solution of compound 128F (14 g, 18.9 mmol) was added TFA (700 mL) at 25° C., the mixture stirred at 25° C. for 1 h under a nitrogen atmosphere and concentrated under reduced pressure. The residue was purified by prep-HPLC (column: Phenomenex C18 250*70 mm 10u; mobile phase: [water (TFA)-ACN]; B %: 0%-30%, 20 min) to give compound 128G TFA salt (6.19 g, 34% yield, 4TFA); ¹H NMR (400 MHz, D₂O) δ 4.11 (br d, J=15.0 Hz, 3H), 3.84 (br d, J=15.6 Hz, 4H), 3.70-3.31 (m, 5H), 3.18 (br d, J=17.6 Hz, 5H), 2.91 (br d, J=10.8 Hz, 2H), 2.86-2.72 (m, 1H), 1.95 (br dd, J=7.4 Hz, 4H), 1.59-1.19 (m, 4H), 1.13-0.82 (m, 12H); and compound 128G (1.61 g, 3.12 mmol, 17% yield) as a white solid; ¹H NMR (400 MHz, D₂O) δ 4.04 (br s, 3H), 3.85 (br d, J=18.2 Hz, 4H), 3.70-3.32 (m, 5H), 3.18 (brd, J=17.6 Hz, 5H), 2.90 (br d, J=10.0 Hz, 2H), 2.86-2.74 (m, 1H), 1.95 (brd, J=5.6 Hz, 4H), 1.42 (br s, 4H), 1.11-0.85 (m, 12H).

### 4.123 Preparation of Compound 128I

Compound 128H (75 mg, 61.4 μmol, 1 eq) was added to a solution of compound 128G (35 mg, 67.7 μmol, 1.1 eq), EDCl (30 mg, 156 μmol, 2.6 eq), HOBt (15 mg, 98 μmol, 1.6 eq), DIEA (70 μL, 402 μmol, 6.5 eq), and DMF (1 mL) at 0° C. The mixture stirred at 0° C. to room temperature over 1 h and then purified by prep-HPLC (30-50% CH₃CN in water with 0.1% TFA) to give compound 128I (13 mg, 13%) as a white solid; LCMS [M/2+H]: 803.5.

### 4.124 Preparation of Compound 128

Compound 128I (13 mg, 8 μmol, 1 eq) was dissolved in a solution of trifluoroacetic acid (0.2 mL), tris(propan-2-yl)silane (0.2 mL) and DCM (0.6 mL). The mixture was stirred at room temperature overnight, concentrated under reduced pressure and purified by prep-HPLC (20-40% CH₃CN in water with 0.1% TFA) to give Compound 128 (7 mg, 54%); LCMS [M/2+H]: 747.4.

Compounds in table 9.21 were prepared by procedures depicted in Scheme D27.

TABLE 9

| Compound | m/z |
|---|---|
| 128.1 | [M/2 + H]: 745.0 |

### D29. Synthesis of Compound 129

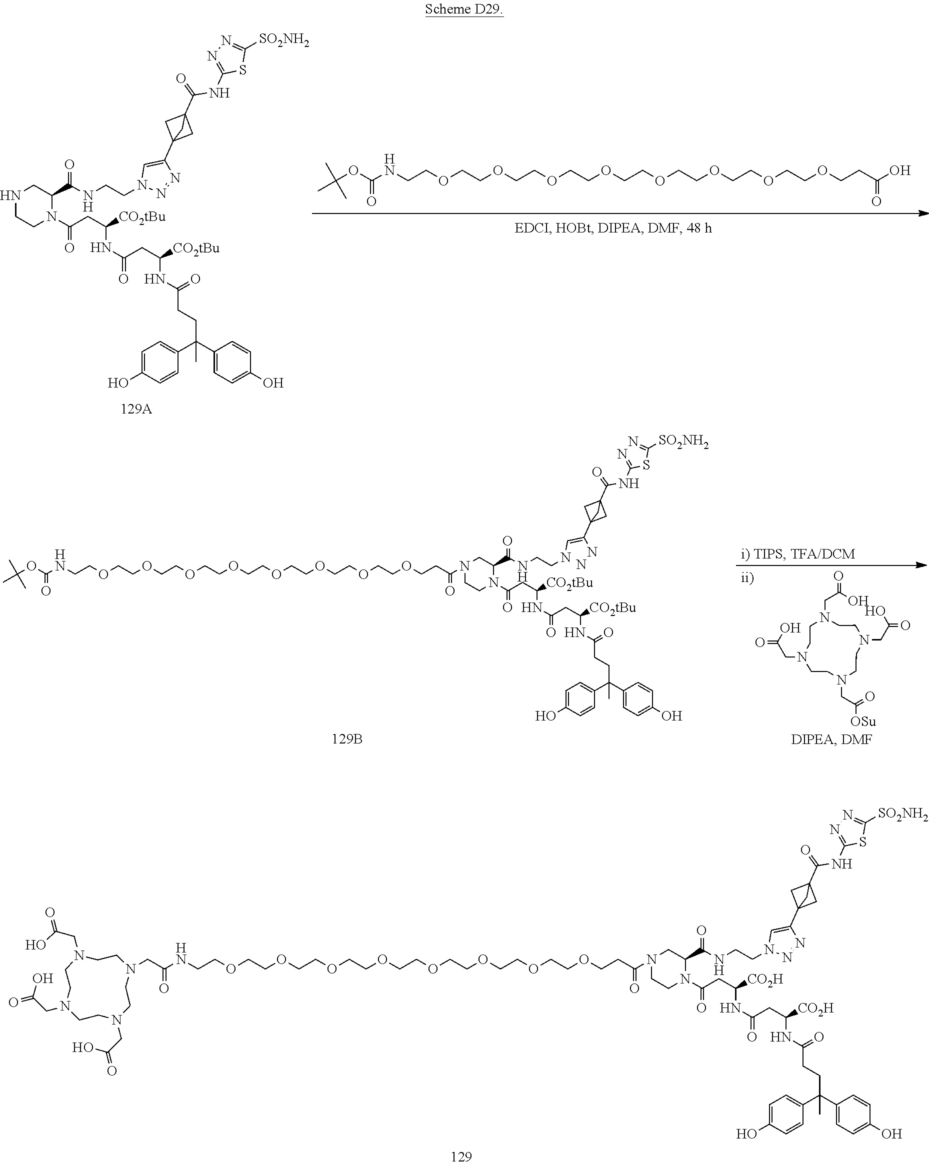

### 4.125 Preparation of Compound 129B

3-{[(Ethylimino)methylidene]amino}-N,N-dimethylpropan-1-amine (32.6 mg, 170 μmol, 2.3 eq) was added to 1-{[(tert-butoxy)carbonyl]amino}-3,6,9,12,15,18,21,24-octaoxaheptacosan-27-oic acid (44 mg, 81.2 μmol, 1.1 eq) in DMF (1.5 mL, 19.4 mmol). 1H-1,2,3-Benzotriazol-1-ol (14 mg, 103 μmol, 1.4 eq) and ethylbis(propan-2-yl)amine (0.39 mL, 222 μmol, 3 eq) were added and the reaction stirred at room temperature for 15 min. Compound 129A (82 mg, 73.9 μmol, 1 eq) was added, the reaction stirred at room temperature for 48 h and directly purified by prep-HPLC (C18, Luna 30×150 mm, 30-70% CH₃CN/H₂O+0.1% Formic acid) to give compound 129B as a white foam (26 mg, 22% yield); LCMS ([M−Boc]-[/Bu])/2+H): 738.0.

### 4.126 Preparation of Compound 129

Compound 129 was prepared from compound 129B as previously depicted; LCMS [M/2+H]: 902.8.

Compounds in table 9.22 were prepared by procedures depicted in Scheme D28.

TABLE 9

| Compound | m/z |
|---|---|
| 129.1 | [M/2 + H]: 900.5 |
| 129.2 | [M/2 + H]: 768.5 |
| 129.3 | [M/2 + H]: 751.5 |
| 129.4 | [M/2 + H]: 768.6 |
| 129.5 | [M/2 + H]: 907.7 |
| 129.6 | [M/2 + H]: 772.1 |
| 129.7 | [M/2 + H]: 765.1 |

### D30. Synthesis of Compound 130

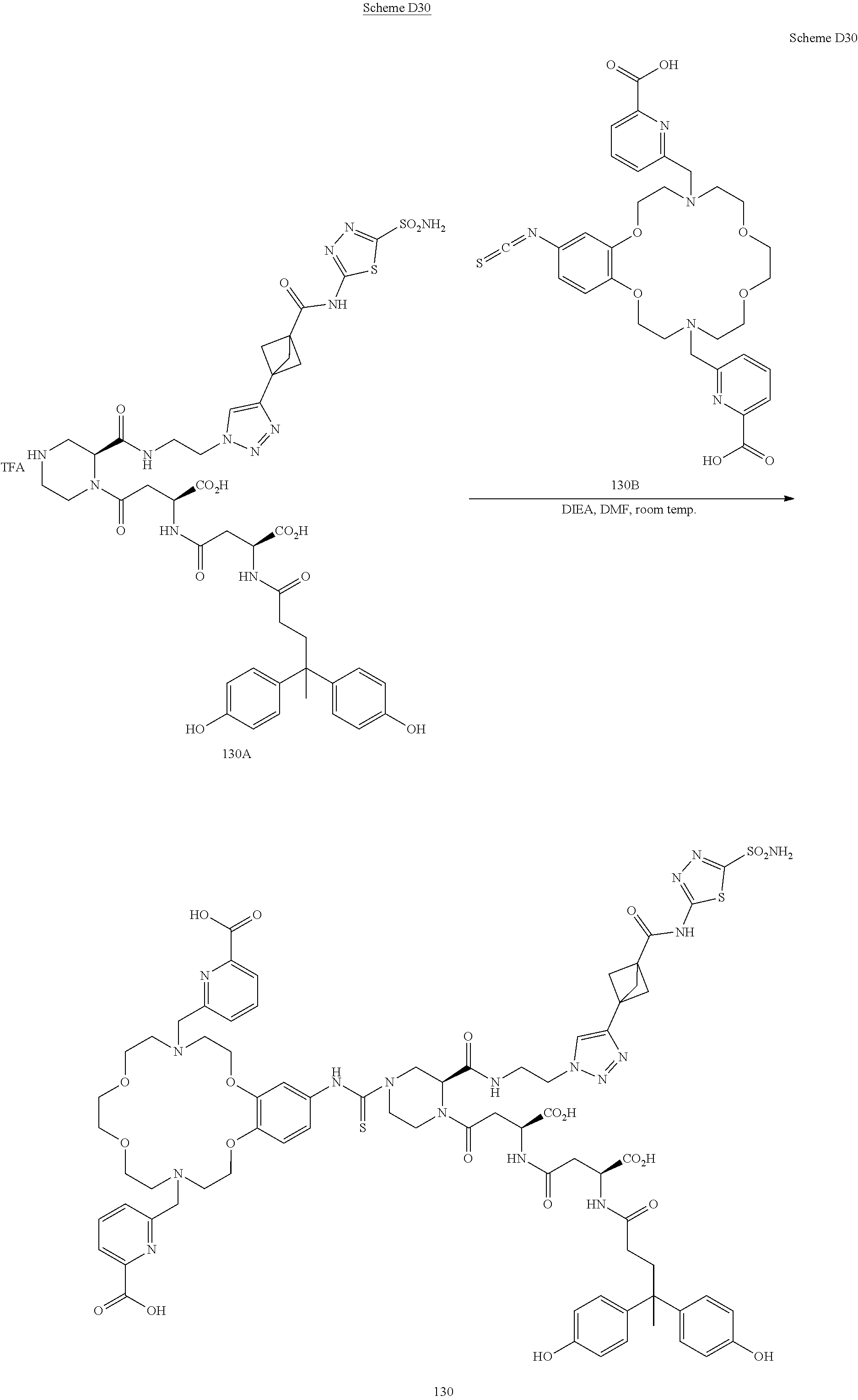 [SMILES: CC(CCC(=O)N[C@@H](CC(=O)N[C@@H](CC(=O)N1CCNC[C@H]1C(=O)NCCn1cc(C23CC(C(=O)Nc4nnc(S(N)(=O)=O)s4)(C2)C3)nn1)C(=O)O)C(=O)O)(c1ccc(O)cc1)c1ccc(O)cc1 ; O=C(O)c1cccc(CN2CCOCCOCCN(Cc3cccc(C(=O)O)n3)CCOc3cc(N=C=S)ccc3OCC2)n1 ; CC(CCC(=O)N[C@@H](CC(=O)N[C@@H](CC(=O)N1CCN(C(=S)Nc2ccc3c(c2)OCCN(Cc2cccc(C(=O)O)n2)CCOCCOCCN(Cc2cccc(C(=O)O)n2)CCO3)C[C@H]1C(=O)NCCn1cc(C23CC(C(=O)Nc4nnc(S(N)(=O)=O)s4)(C2)C3)nn1)C(=O)O)C(=O)O)(c1ccc(O)cc1)c1ccc(O)cc1]

Compound 130A (30 mg, 27 μmol) and compound 130B (35 mg, 54 μmol) were dissolved in DMF (450 μL) and DIEA (75 μL) and the reaction mixture stirred at room temperature overnight. The reaction was purified directly by prep. HPLC (10-40% ACN/H₂O gradient, TFA modifier) to give Compound 130 (17.5 mg, 40% yield); LCMS [M/2+H]: 816.2.

### D31. Synthesis of Compound 131

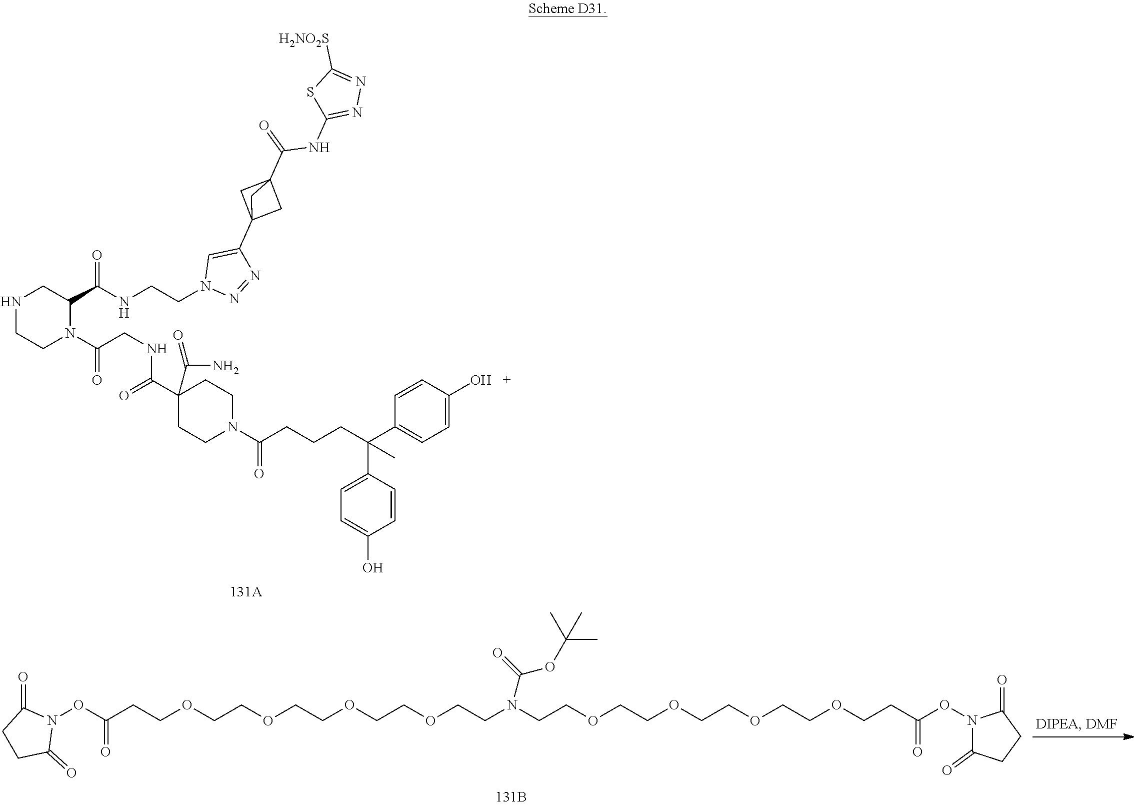

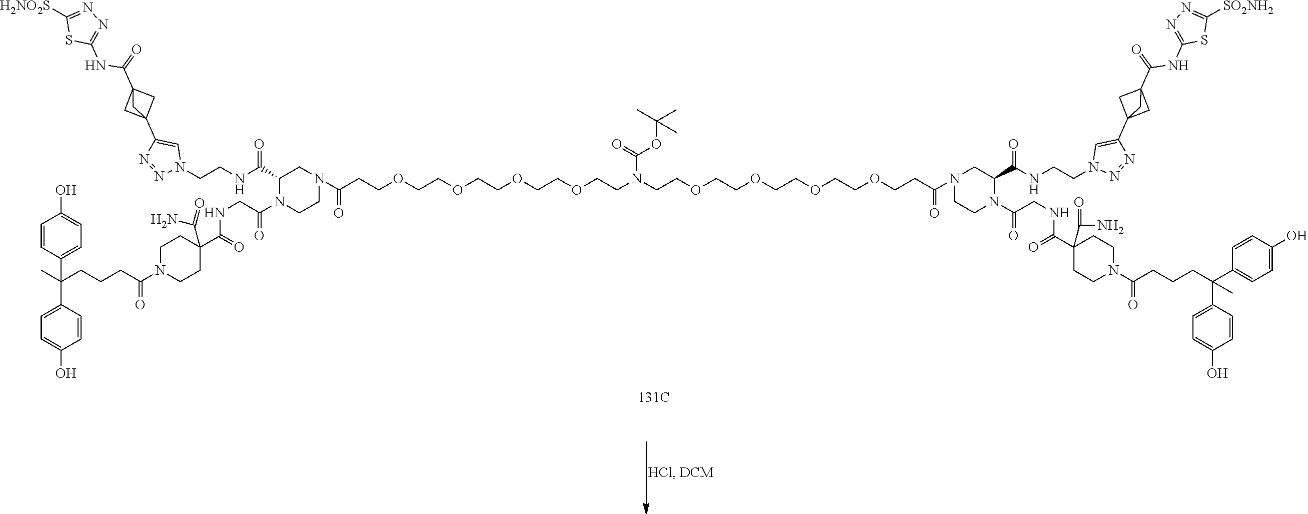 [SMILES: CC(C)(C)OC(=O)N(CCOCCOCCOCCOCCC(=O)N1CCN(C(=O)CNC(=O)C2(C(N)=O)CCN(C(=O)CCCC(C)(c3ccc(O)cc3)c3ccc(O)cc3)CC2)[C@H](C(=O)NCCn2cc(C34CC(C(=O)Nc5nnc(S(N)(=O)=O)s5)(C3)C4)nn2)C1)CCOCCOCCOCCOCCC(=O)N1CCN(C(=O)CNC(=O)C2(C(N)=O)CCN(C(=O)CCCC(C)(c3ccc(O)cc3)c3ccc(O)cc3)CC2)[C@H](C(=O)NCCn2cc(C34CC(C(=O)Nc5nnc(S(N)(=O)=O)s5)(C3)C4)nn2)C1]

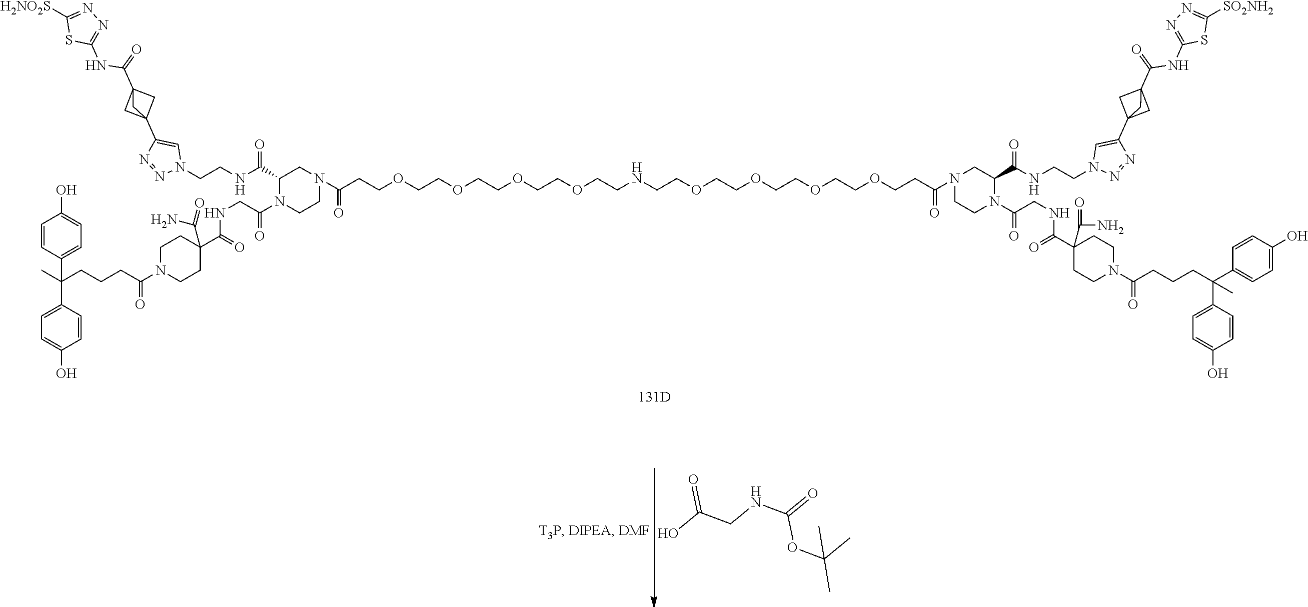 [SMILES: CC(CCCC(=O)N1CCC(C(N)=O)(C(=O)NCC(=O)N2CCN(C(=O)CCOCCOCCOCCOCCNCCOCCOCCOCCOCCC(=O)N3CCN(C(=O)CNC(=O)C4(C(N)=O)CCN(C(=O)CCCC(C)(c5ccc(O)cc5)c5ccc(O)cc5)CC4)[C@H](C(=O)NCCn4cc(C56CC(C(=O)Nc7nnc(S(N)(=O)=O)s7)(C5)C6)nn4)C3)C[C@H]2C(=O)NCCn2cc(C34CC(C(=O)Nc5nnc(S(N)(=O)=O)s5)(C3)C4)nn2)CC1)(c1ccc(O)cc1)c1ccc(O)cc1 ; CC(C)(C)OC(=O)NCC(=O)O]

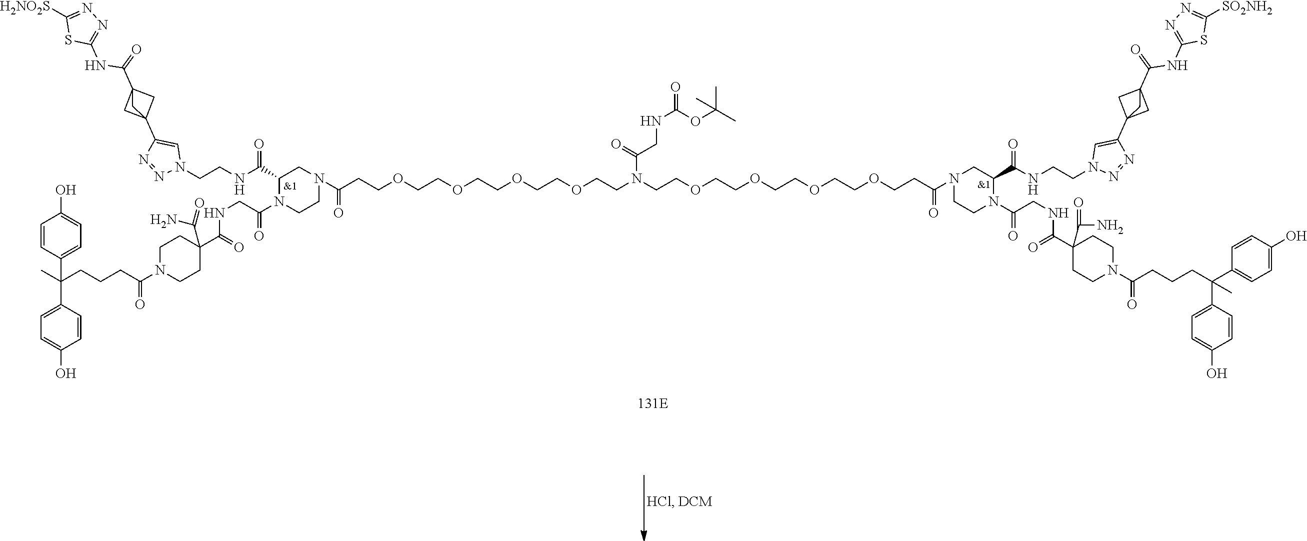 [SMILES: CC(C)(C)OC(=O)NCC(=O)N(CCOCCOCCOCCOCCC(=O)N1CCN(C(=O)CNC(=O)C2(C(N)=O)CCN(C(=O)CCCC(C)(c3ccc(O)cc3)c3ccc(O)cc3)CC2)[C@H](C(=O)NCCn2cc(C34CC(C(=O)Nc5nnc(S(N)(=O)=O)s5)(C3)C4)nn2)C1)CCOCCOCCOCCOCCC(=O)N1CCN(C(=O)CNC(=O)C2(C(N)=O)CCN(C(=O)CCCC(C)(c3ccc(O)cc3)c3ccc(O)cc3)CC2)[C@H](C(=O)NCCn2cc(C34CC(C(=O)Nc5nnc(S(N)(=O)=O)s5)(C3)C4)nn2)C1]

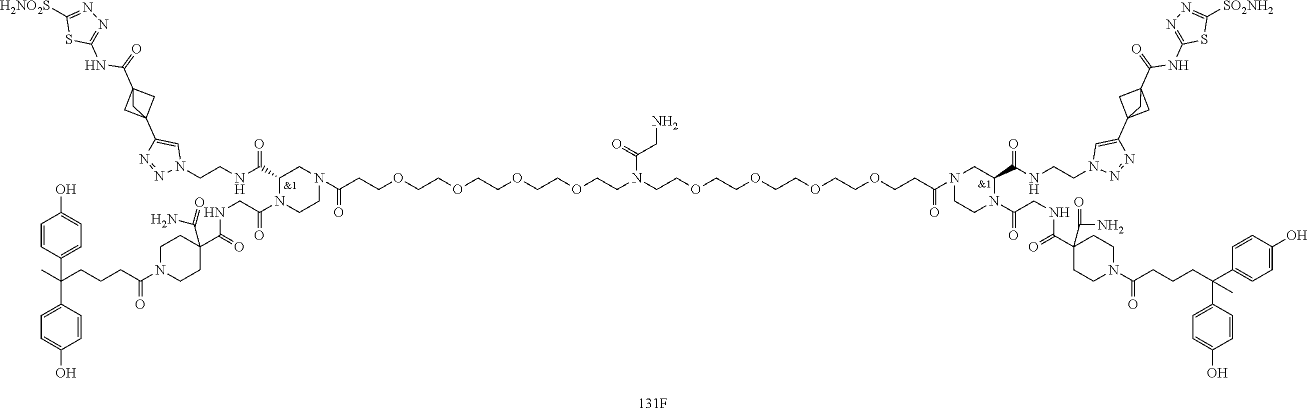 [SMILES: CC(CCCC(=O)N1CCC(C(N)=O)(C(=O)NCC(=O)N2CCN(C(=O)CCOCCOCCOCCOCCN(CCOCCOCCOCCOCCC(=O)N3CCN(C(=O)CNC(=O)C4(C(N)=O)CCN(C(=O)CCCC(C)(c5ccc(O)cc5)c5ccc(O)cc5)CC4)[C@H](C(=O)NCCn4cc(C56CC(C(=O)Nc7nnc(S(N)(=O)=O)s7)(C5)C6)nn4)C3)C(=O)CN)C[C@H]2C(=O)NCCn2cc(C34CC(C(=O)Nc5nnc(S(N)(=O)=O)s5)(C3)C4)nn2)CC1)(c1ccc(O)cc1)c1ccc(O)cc1]

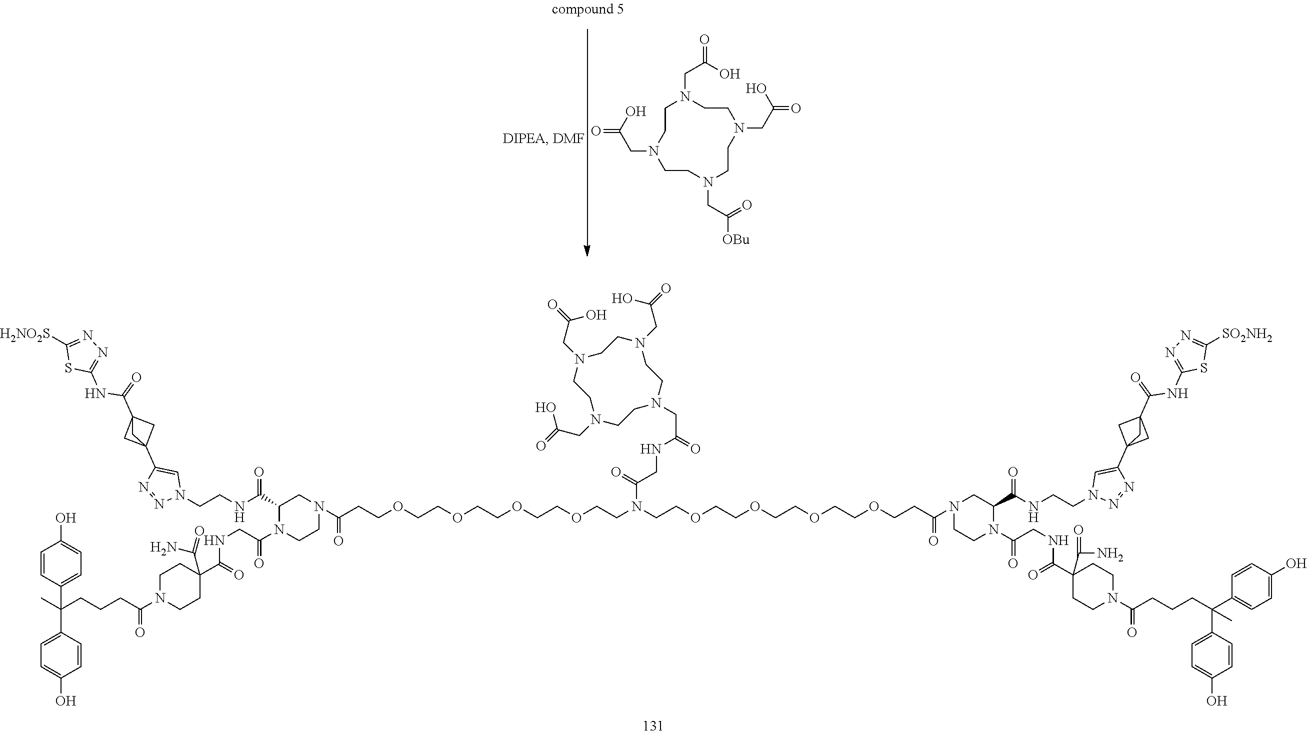

### 4.128 Preparation of Compound 131C

To a solution of bis {2-[2-(2-{2-[3-(2,5-dioxo-1-pyrrolidinyloxy)-3-oxopropoxy]ethoxy}ethoxy)ethoxy]ethyl}amino-tert-butylformylate (38 mg, 47 μmol, 1 eq) in DMF (1.5 mL) was added compound 131A (182 mg, 165 μmol, 3.5 eq) followed by ethylbis(propan-2-yl)amine (369 μL, 2.1 mmol). The resulting solution stirred at room temperature for 16 h and purified directly by prep-HPLC (C18, Luna 30×150 mm, 30-70% CH₃CN/H₂O+0.1% Trifluoroacetic acid) to give compound 131C as a white solid (38 mg, 32% yield); LCMS [M-Boc/2+H]: 1229.2.

### 4.129 Preparation of Compound 131D

4N HCl in dioxane (20 μl, 14.9 μmol, 5 eq) was added to a solution of compound 131C (38 mg, 14.9 μmol) in DCM (1 mL) and stirred at room temperature for 6 h. The reaction was concentrated under reduced pressure to give crude compound 131D (33 mg) as a white solid; LCMS [M/2+H]: 1229.2.

### 4.130 Preparation of Compound 131E

Compound 131D (33 mg, 9.0 μmol) was taken up in DMF (1.5 mL), (tert-butoxycarbonylamino) acetic acid (2.8 mg, 15.9 μmol, 1.2 eq) followed by N-ethylbis(isopropyl)amine (7.0 μL, 40 μmol, 3 eq) and 2,4,6-tripropyl-1,3,5,225,425,625-trioxatriphosphinane-2,4, 6-trione (10 μL, 33 μmol, 2.5 eq) were added dropwise. The resulting solution was stirred at room temperature for 0.5 h and purified directly by prep-HPLC (C18, Luna 30×150 mm, 25-60% CH₃CN/H₂O+ 0.1% TFA) to give compound 131E as a white solid (25 mg, 72% yield); LCMS [M−Boc/3+H]: 838.9.

### 4.131 Preparation of Compound 131F

4N HCl in dioxane (12 μl, 48 μmol, 5 eq) was added to a solution of compound 131E (25 mg, 10 μmol) in DCM (1 mL) and stirred at room temperature for 16 h. The reaction was concentrated under reduced pressure to give crude compound 131F as white solid (10 mg); LCMS [M/2+H]: 838.7.

### 4.132 Preparation of Compound 131

Compound 131 was prepared from compound 131F using procedures previously depicted. LCMS [M/3+H]: 967.8.

Compounds in table 9.23 were prepared by procedures depicted in Scheme D29.

TABLE 9

| Compound | m/z |
|---|---|
| 131.1 | [M/3 + H]: 952.2 |
| 131.2 | [M/3 + H]: 970.3 |
| 131.3 | [M + H]/3: 978.3 |
| 131.4 | [M/3 + H]: 839.2 |
| 131.5 | [M/3 + H]: 792.1 |
| 131.6 | [M/3 + H]: 977.6 |
| 131.7 | [M/3 + H]: 977.2 |

### D32. Synthesis of Compound 132

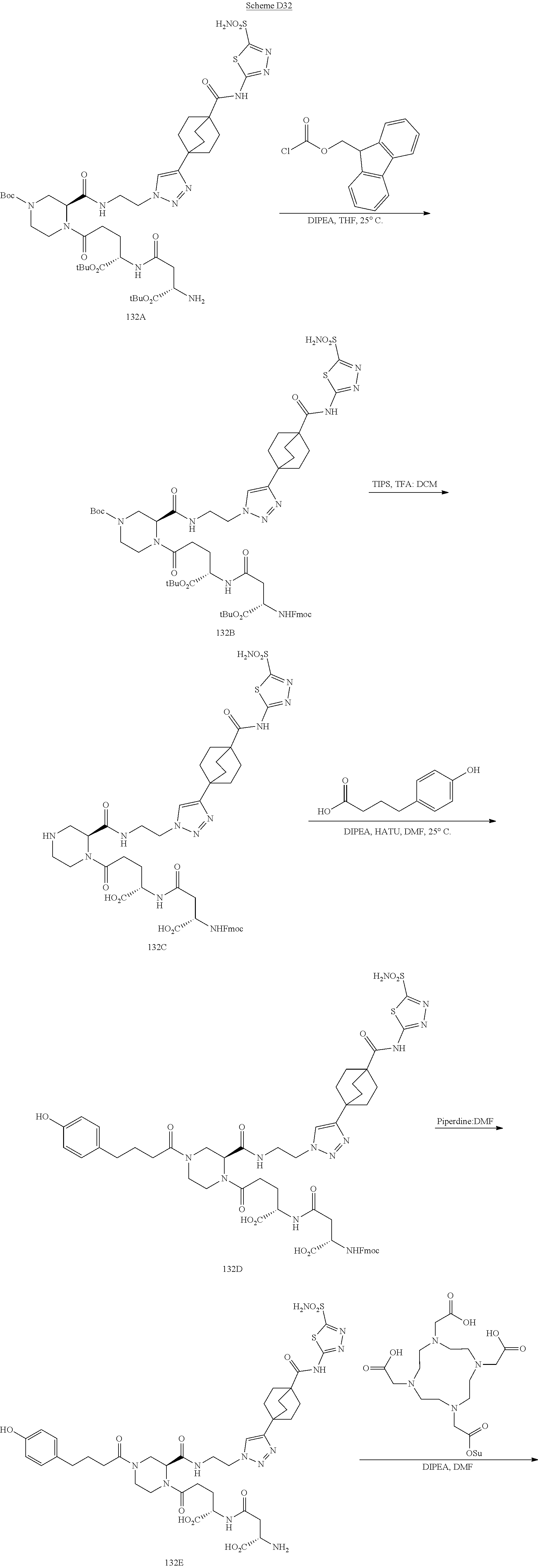

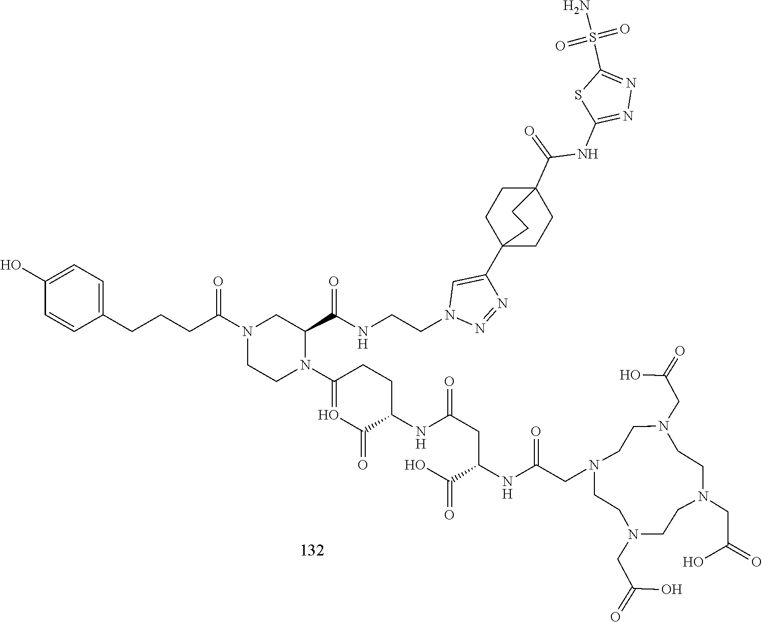

### 4.135 Preparation of Compound 132B

(9H-fluoren-9-yl)methyl carbonochloridate (27 mg, 112 μmol, 1.1 eq) and DIPEA (37 μl, 212 μmol, 2.1 eq) were added to a stirred solution of compound 132A (100 mg, 0.1 mmol, 1 eq) in THF (1 mL). The reaction was stirred at room temperature for 30 min, diluted with EtOAc, washed with water and brine, dried (Na₂SO₄), filtered and to give crude compound 132B (122 mg) that was used directly in the next reaction; [M+H]: 1217.4.

### 4.136 Preparation of Compound 132C

Tris(isopropyl)silane (0.8 mL) and TFA (0.8 mL) were added to a stirred solution of compound 132B (122 mg, 0.1 mmol, 1 eq) in DCM (0.6 mL). The reaction was stirred at room temperature for 17 h and concentrated under reduced pressure. The reaction was purified by prep-HPLC (Luna C18 30×150 mm column, 25-50% ACN/H2O+ 0.1% TFA) to give compound 132C (69 mg) as a white powder; [M+H]: 1005.4.

### 4.137 Preparation of Compound 132D

HATU (20 mg, 52.5 μmol, 1.2 eq) and DIPEA (23 μl, 131 μmol, 3 eq) were added to a stirred solution of 4-(4-hydroxyphenyl) butanoic acid (8 mg, 44 μmol, 1 eq) in DMF (1.2 mL). The reaction solution was stirred at room temperature for 20 min, compound 132C (44 mg, 43 μmol, 1 eq) added and the crude reaction stirred at room temperature for 16 h. The reaction was quenched with 1N HCl (10 mL), the aqueous layer extracted with EtOAc (2×8 mL), the combined organics washed with brine (10 mL), dried (Na₂SO₄), filtered and concentrated under reduced pressure to give crude compound 132D (38 mg, 74% yield); [M+H]: 1168.3.

### 4.138 Preparation of Compound 132E

Piperidine (5.0 μl, 48.8 μmol, 1.5 eq) was added to a stirred solution of compound 132D (38 mg, 32.6 μmol, 1 eq) in DMF (0.5 mL). The reaction was stirred at room temperature for 0.5 h and purified directly by prep-HPLC (Luna C18 30×150 mm column, 0-50% ACN/H₂O+ 0.1% TFA) to give compound 132E (20 mg, 65% yield); [M+H]: 946.3.

### 4.139 Preparation of Compound 132

Compound 132 was prepared from compound 132E using procedures previously depicted; LCMS [M/2+H]: 666.2.

### D33. Synthesis of Compound 133

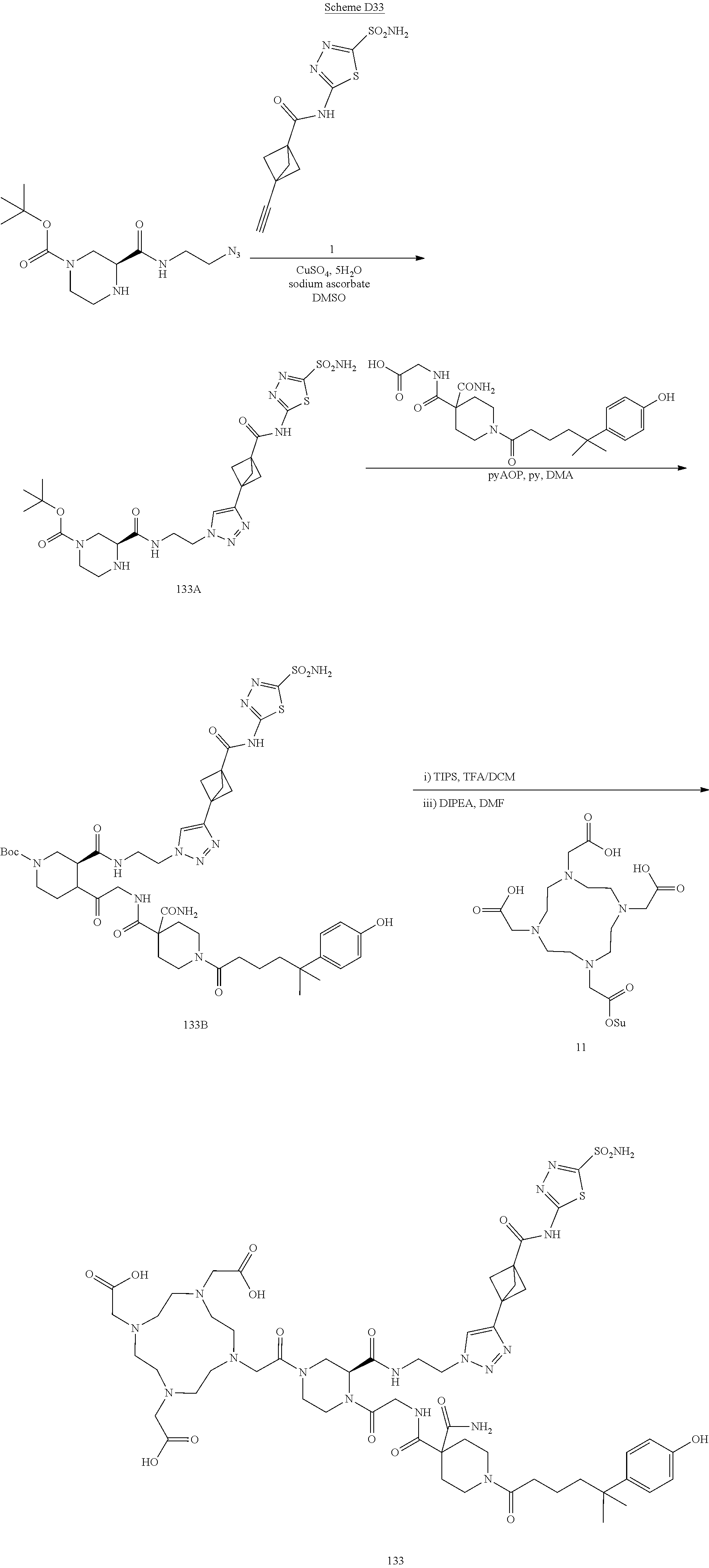

### 4.140 Preparation of Compound 133A

To a solution of tert-butyl(S)-3-((2-azidoethyl)carbamoyl)piperazine-1-carboxylate (85.0 g, 279 mmol) and compound 1 (68.3 g, 228 mmol) in DMSO (845 mL) was added CuSO₄·5H₂O (3.49 g, 13.9 mmol) and sodium ascorbate (55.4 g, 279 mmol). The mixture was stirred at 25° C. for 8 h. The reaction mixture was quenched by addition of H₂O (700 mL) at 25° C., and extracted with DCM (5×500 mL). The combined organic layers were filtered and concentrated under reduced pressure. The residue was added dropwise to H₂O (3000 mL) at 5° C. and the green solid was collected by filtration. The crude product was triturated with MeOH (750 mL) at 25° C. for 12 h to give compound 133A (132 g) as an off-white solid; LCMS [M+H]+=597.3; ¹H NMR (400 MHz, CD₃OD) δ 7.91 (s, 1H), 4.56 (m, 2H), 3.77-3.75 (m, 1H), 3.70-3.67 (m, 1H), 2.96-2.93 (m, 3H), 2.67-2.65 (m, 1H), 2.52 (s, 6H), 1.46 (s, 9H).

### 4.141 Preparation of Compound 133B

To a stirred solution of (4-carbamoyl-1-(5-(4-hydroxyphenyl)-5-methylhexanoyl) piperidine-4-carbonyl)glycine (141 mg, 0.33 mmol) in anhydrous DMA (1.5 mL), PyAOP (282 mg, 0.54 mmol) was added followed by pyridine (0.70 mL, 8.7 mmol) and the resulting heated to 40° C., Compound 133A (130 mg, 0.22 mmol) was slowly added to the solution portion wise over 5 min. The resulting solution was stirred at room temperature for 1 h and directly purified by prep-HPLC to afford compound 133B as a white solid (180 mg, 82% yield); [M+H]+=1012.5.

### 4.142 Preparation of Compound 133

Compound 133 was prepared from Compound 133B as previously depicted; LCMS [M/2+H]: 650.0.

### D34. Synthesis of Compound 134

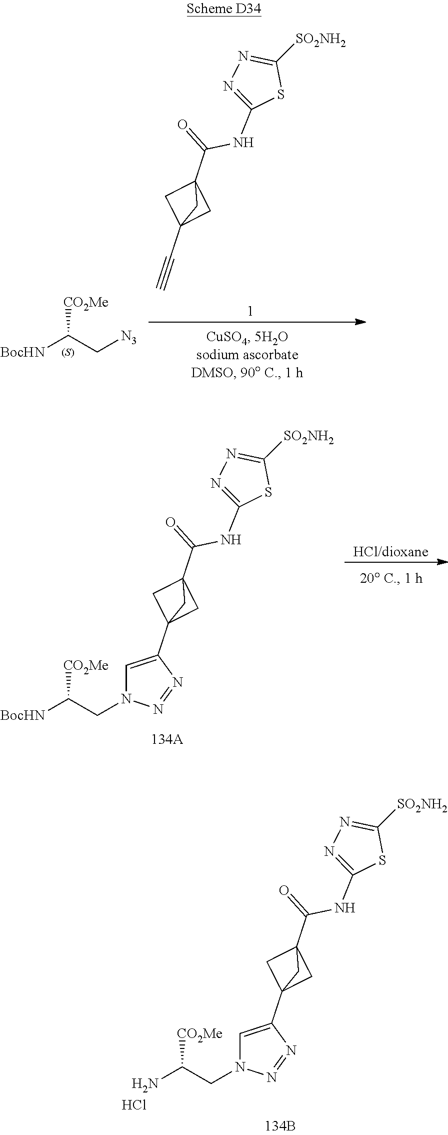

### Preparation of Compound 134A

To a solution of methyl(S)-3-azido-2-((tert-butoxycarbonyl)amino)propanoate (200 mg, 819 μmol) and compound 1 (244 mg, 819 μmol) in DMSO (2 mL) was added CuSO₄·5H₂O (41 mg, 164 μmol) and sodium ascorbate (324 mg, 1.64 mmol). The mixture was stirred at 90° C. for 1 h. The crude product was triturated with H₂O (8 mL) and NH₄Cl (2 mL) at 20° C. for 10 min, the filter cake washed with H₂O (10 mL), and the filter cake concentrated under reduced pressure to give crude compound 134A (450 mg) as a white solid; LCMS [M+H]+=543.2; ¹H NMR (400 MHz, DMSO-d₆) δ 8.49-8.05 (m, 2H), 8.02-7.87 (m, 1H), 7.43 (brd, J=8.0 Hz, 1H), 4.78-4.65 (m, 1H), 4.61-4.48 (m, 2H), 3.66 (s, 3H), 2.42 (br s, 6H), 1.33 (s, 9H).

### Preparation of Compound 134B

A solution of compound 134A (400 mg, 737 μmol) in 4M HCl in dioxane (4 mL) was stirred at 20° C. for 1 h and concentrated under reduced pressure to give crude compound 134B (500 mg, 3HCl) as a white solid; LCMS: [M−1]⁻=441.0; ¹H NMR (400 MHz, DMSO-d₆) δ 13.22 (br s, 1H), 8.92 (br s, 2H), 8.36 (s, 2H), 8.07 (s, 1H), 4.95-4.82 (m, 2H), 4.69 (br s, 1H), 3.78-3.70 (m, 3H), 2.45 (s, 6H).

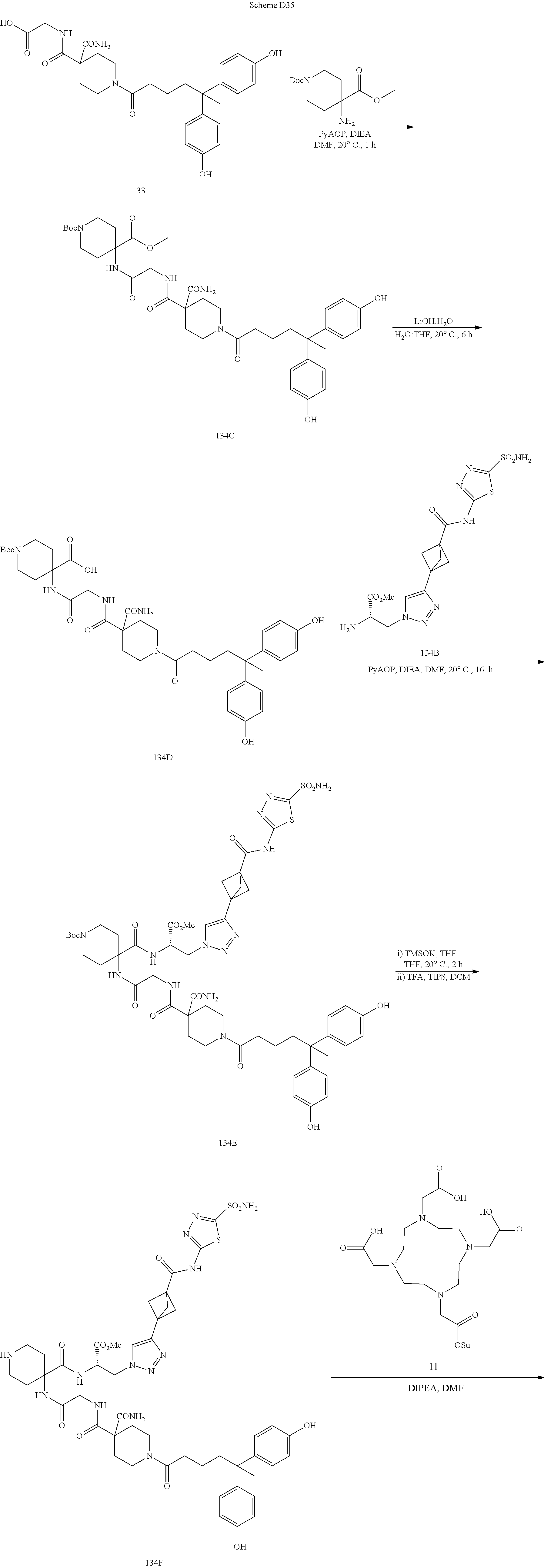

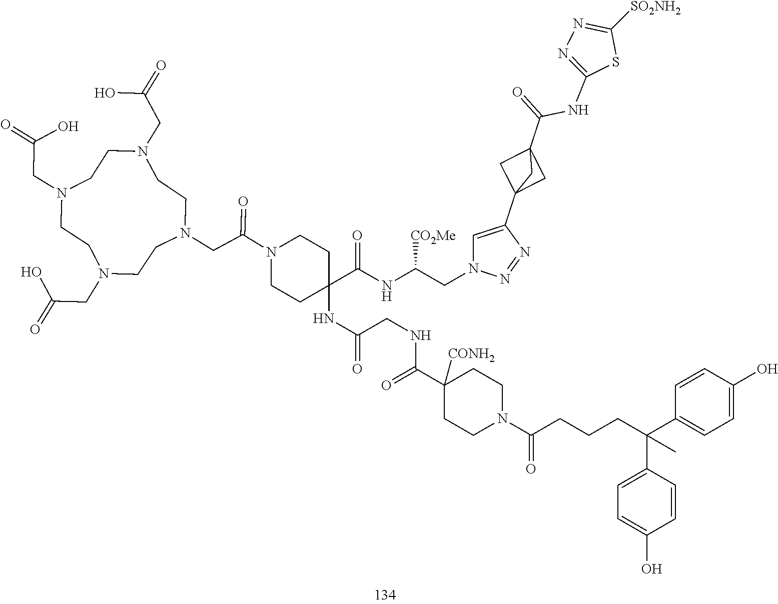

### Preparation of Compound 134C

To a solution of 1-(tert-butyl) 4-methyl 4-aminopiperidine-1,4-dicarboxylate (353 mg, 1.37 mmol) and compound 33 (700 mg, 1.37 mmol) in DMF (10 mL) was added PyAOP (785 mg, 1.51 mmol) and DIEA (715 μL, 4.11 mmol). The mixture was stirred at 20° C. for 1 h, diluted with H₂O (50 mL) and extracted with EtOAc (3×30 mL). The combined organic layers were washed with brine (3×10 mL), dried (Na₂SO₄), filtered and concentrated under reduced pressure. The crude product was purified by column chromatography (SiO₂, MeOH:DCM) to give crude compound 134C (825 mg) as a white solid:LCMS [M+H]+=752.3.

### Preparation of Compound 134D

To a solution of compound 134C (700 mg, 931 μmol) in THF (3.5 mL) and H₂O (3.5 mL) was added LiOH·H₂O (117 mg, 2.79 mmol). The mixture was stirred at 20° C. for 6 h and organics removed under reduced pressure. The reaction mixture was adjusted to pH=4, the solid separated and concentrated under reduced pressure to give compound 134D (680 mg, 99% yield) as a white solid: LCMS [M+H]+=738.6.

### Preparation of Compound 134E

To a solution of compound 134D (484 mg, 655 μmol) and compound 134B (580 mg, 1.31 mmol) in DMF (4.8 mL) was added PyAOP (376 mg, 721 μmol) and DIEA (342 μL, 1.97 mmol). The mixture was stirred at 20° C. for 16 h. The reaction mixture was concentrated under reduced pressure to give crude compound 134E (761 mg) as a yellow oil: LCMS [M+H]+=1162.7.

### Preparation of Compound 134F

To a solution of compound 134E (740 mg, 637 μmol) in THF (8 mL) was added TMSOK (408 mg, 3.18 mmol). The mixture was stirred at 20° C. for 1 h, the reaction mixture adjusted to pH=6 and concentrated under reduced pressure. The residue was taken up in DCM (4 mL), triisopropylsilane (2.58 mL, 12.5 mmol) and TFA (4 mL) added and the reaction stirred at 20° C. for 1 h. The reaction mixture was concentrated under reduced pressure and purified by prep-HPLC to give compound 134F (81 mg, 12% yield) as a white solid; LCMS [M+H]+=1048.6; ¹H NMR (400 MHz, DMSO-d₆) δ 13.19 (s, 2H), 9.12 (s, 2H), 8.54 (br d, J=3.8 Hz, 1H), 8.35 (s, 2H), 8.32-8.21 (m, 2H), 8.07 (br d, J=7.4 Hz, 1H), 7.89 (d, J=2.6 Hz, 1H), 7.37 (brd, J=12.0 Hz, 2H), 6.93 (br d, J=8.6 Hz, 4H), 6.62 (d, J=8.6 Hz, 4H), 4.81 (br d, J=10.2 Hz, 1H), 4.66-4.48 (m, 2H), 3.79 (br d, J=4.4 Hz, 3H), 3.51 (br s, 1H), 3.18 (br s, 3H), 3.08-2.84 (m, 3H), 2.42 (s, 6H), 2.31-2.15 (m, 3H), 2.02 (brd, J=11.4 Hz, 5H), 1.96-1.73 (m, 4H), 1.47 (s, 3H), 1.23 (br s, 2H).

### Preparation of Compound 134

Compound 134 was prepared from compound 134F using procedures previously depicted; LCMS [M+H]+=1434.9.

### D35. Synthesis of Compound 135

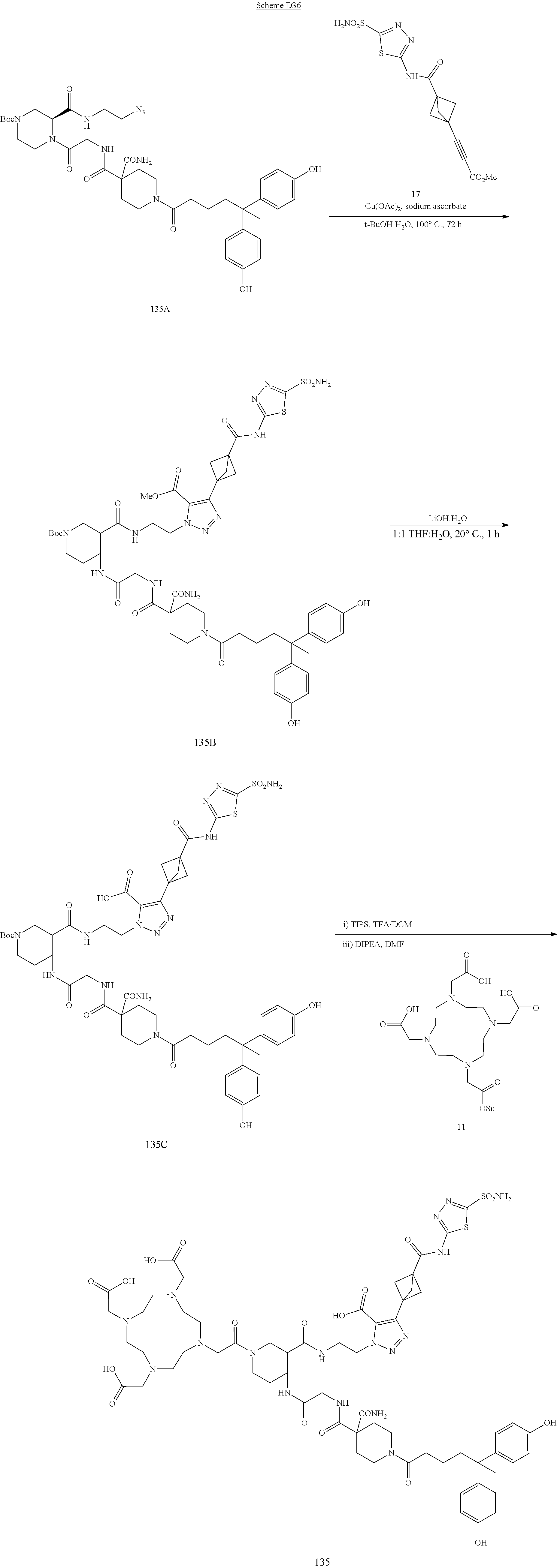

### Preparation of Compound 135B

To a solution of compound 17 (747 mg, 2.10 mmol) and compound 135A (1.3 g, 1.61 mmol) in t-BuOH (7 mL) and H₂O (0.7 mL) was added Cu(OAc)₂ (117 mg, 645 μmol) and sodium ascorbate (256 mg, 1.29 mmol) in t-BuOH (7 mL) under a nitrogen atmosphere at 20° C. The mixture was stirred at 100° C. for 72 h and concentrated under reduced pressure. The residue was purified by prep-HPLC followed by prep-SFC to separate the triazole regioisomers to give compound 135B (900 mg, 60%) as a white solid; LCMS [M+H]+=1162.4; ¹H NMR (400 MHz, DMSO-d₆) δ 12.93-13.46 (m, 1H), 9.12 (s, 2H), 8.13-8.36 (m, 3H), 8.06 (s, 1H), 7.64 (brt, J=5.8 Hz, 1H), 7.22-7.43 (m, 2H), 6.93 (d, J=8.6 Hz, 4H), 6.63 (d, J=8.6 Hz, 4H), 4.65 (br t, J=5.8 Hz, 2H), 3.94 (s, 3H), 3.70-3.85 (m, 5H), 3.57-3.68 (m, 2H), 3.42-3.56 (m, 3H), 3.07-3.14 (m, 1H), 2.82-3.03 (m, 3H), 2.24 (brt, J=7.2 Hz, 2H), 1.89-2.10 (m, 4H), 1.70-1.88 (m, 4H), 1.63 (brt, J=10.6 Hz, 2H), 1.47 (s, 3H), 1.39 (s, 9H), 1.20-1.25 (m, 2H).

### Preparation of Compound 135C

To a solution of compound 135B (350 mg, 301 μmol) in THF (2 mL) and H₂O (2 mL) was added LiOH·H₂O (51 mg, 1.20 mmol). The mixture was stirred at 20° C. for 1 h and concentrated under reduced pressure to give crude compound 135C (350 mg) as a white solid; LCMS [M+H]+=1148.5; ¹H NMR (400 MHz, DMSO-d₆) δ 12.97-13.32 (m, 1H), 9.02-9.28 (m, 2H), 8.48-8.80 (m, 1H), 8.34 (s, 2H), 7.76-8.09 (m, 1H), 7.53 (br s, 1H), 7.30 (br s, 1H), 6.93 (br d, J=8.41 Hz, 4H), 6.62 (br d, J=8.41 Hz, 4H), 4.46-4.96 (m, 1H), 3.83 (brs, 3H), 3.65 (br s, 2H), 3.02-3.20 (m, 5H), 2.89 (br d, J=6.65 Hz, 4H), 2.42 (br d, J=4.14 Hz, 2H), 2.24 (br s, 2H), 2.15 (brs, 2H), 1.74-2.01 (m, 7H), 1.67 (br s, 2H), 1.46 (br s, 3H), 1.38 (s, 9H), 1.23 (br s, 3H).

### Preparation of Compound 135

Compound 135 was prepared from compound 135C using procedures previously depicted; LCMS [M+H]⁺=1435.0.

### D36. Synthesis of Compound 136

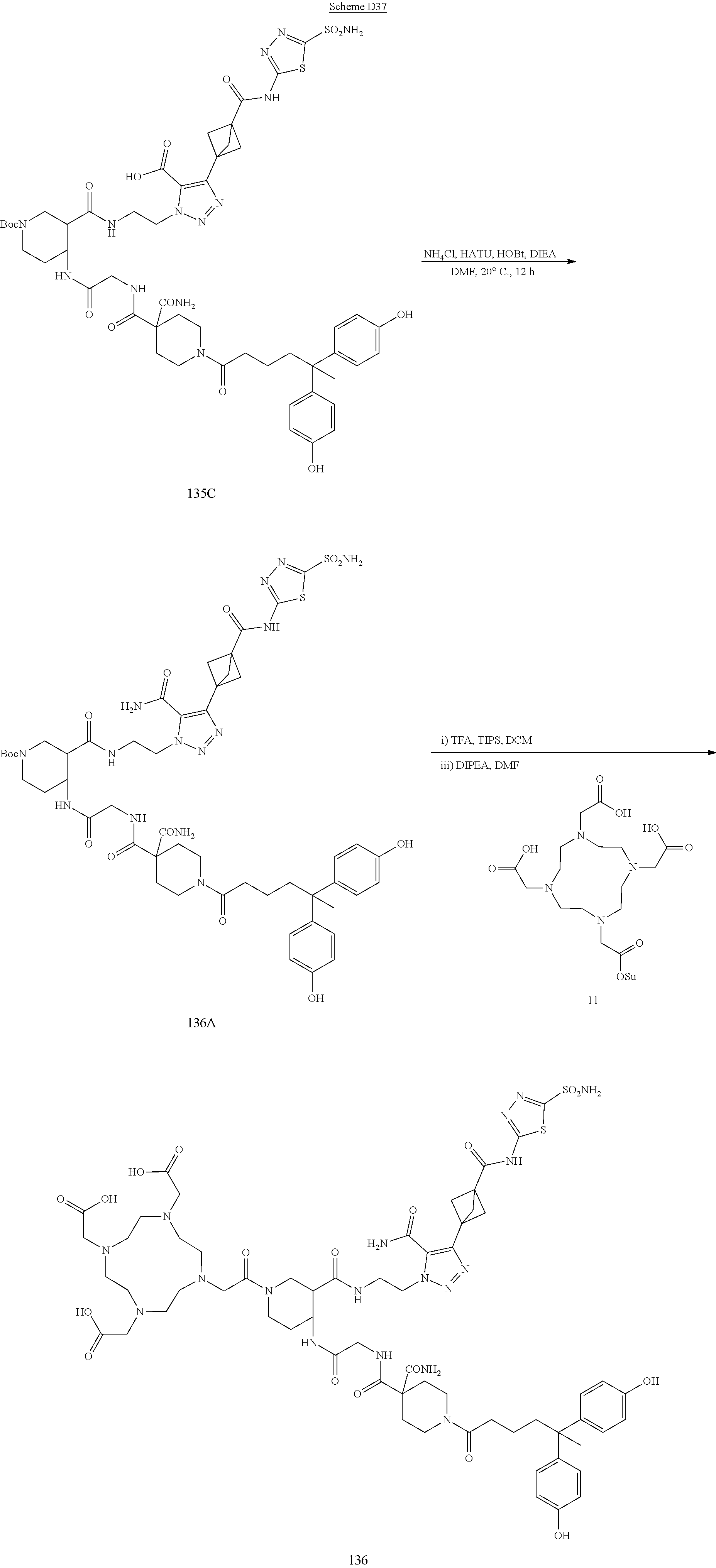

### Preparation of Compound 136A

To a solution of compound 135C (150 mg, 131 μmol) and NH₄Cl (21 mg, 392 μmol) in DMF (1.5 mL) was added DIEA (46 μL, 261 μmol), HOBt (35 mg, 261 μmol) and HATU (99 mg, 261 μmol). The mixture was stirred at 20° C. for 12 h and concentrated under reduced pressure. The crude product was purified by prep-HPLC to give compound 136A (30 mg) as a white solid; LCMS: [M+H]⁺=1147.6.

### Preparation of Compound 136

Compound 136 was prepared from compound 136A using procedures previously depicted; LCMS [M+H]+=1434.1.

### E1. Synthesis of Compound 106-Lu

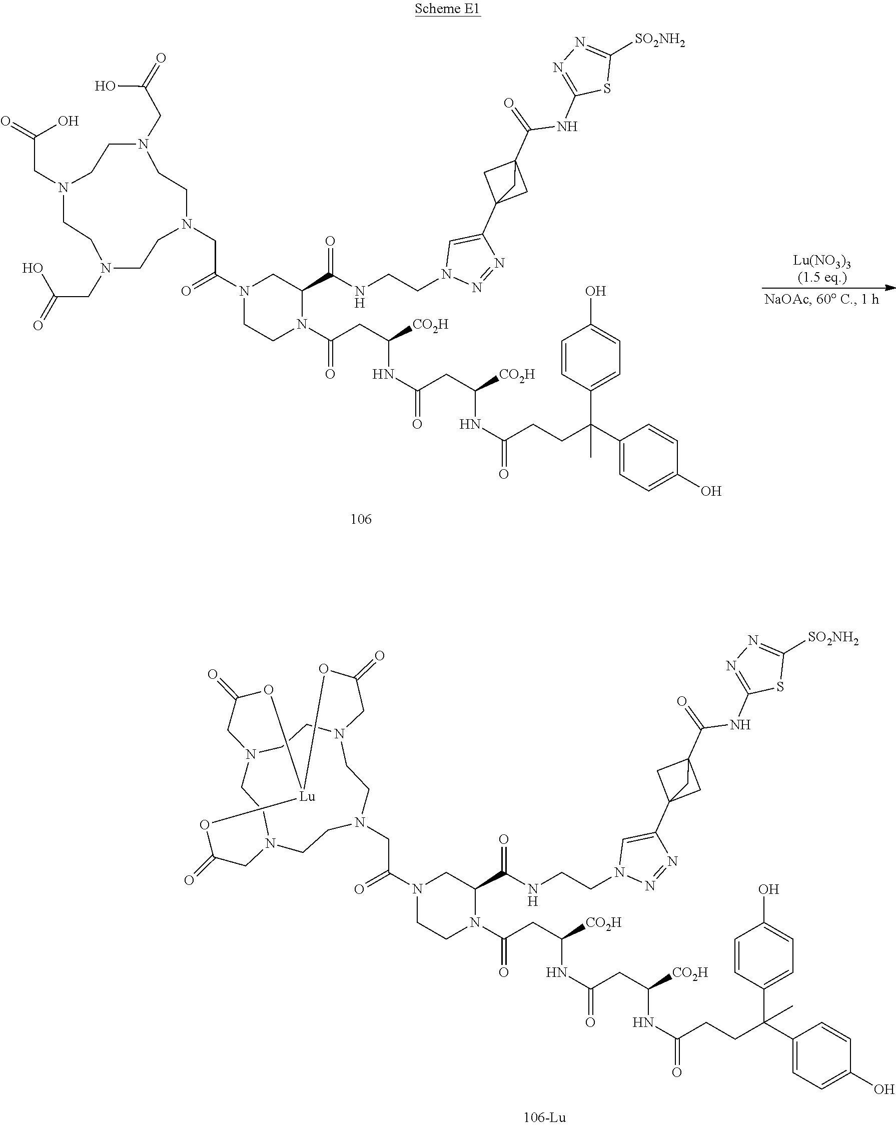

To a solution of Compound 106 (80 mg, 57.9 μmol, 1 eq) in H₂O (1 mL) was added NaOAc (16 mg, 195 μmol, 3.4 eq) and Lu(NO₃)₃ (32.9 mg, 89 μmol, 1.5 eq), the mixture stirred at 60° C. for 1 h and concentrated under reduced pressure. The residue was purified by prep-HPLC (PhenomenexC18 150*30 mm*5 μm; mobile phase: [water (TFA)-ACN]; B %: 10%-30%, 8 min) to give Compound 106-Lu (30.3 mg, 34% yield) as a white solid; LCMS [M+H]: 1553.4; ¹H NMR (400 MHz, DMSO-d₆) δ 13.66-13.16 (m, 1H), 12.86-12.42 (m, 1H), 9.39-9.02 (m, 1H), 8.59-7.61 (m, 5H), 6.91 (b r d, J=8.3 Hz, 4H), 6.64 (b r d, J=7.2 Hz, 4H), 5.07-4.65 (m, 2H), 4.58-4.35 (m, 4H), 4.31-3.91 (m, 5H), 3.43-3.28 (m, 6H), 3.24-3.06 (m, 3H), 2.71-2.69 (m, 1H), 3.04-2.69 (m, 10H), 2.69-2.53 (m, 10H), 2.47-2.40 (m, 6H), 2.35-2.31 (m, 1H), 2.16 (b r t, J=7.1 Hz, 2H), 1.84 (b r d, J=6.5 Hz, 2H), 1.44 (br s, 3H).

Compounds in table 10.1 were prepared by procedures depicted in scheme E1 using the appropriate inorganic salt; Lanthanum trichloride, gallium tris(nitrate) monohydrate or copper dichloride.

TABLE 10.1

| Compound | Structure | m/z |
|---|---|---|
| 106-La | 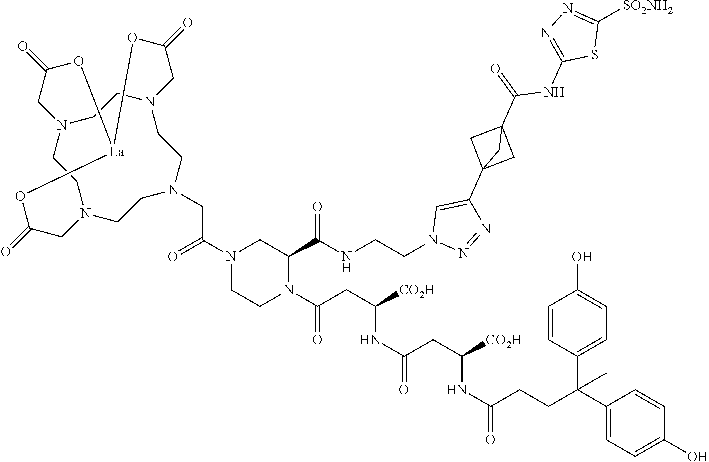 | [M/2 + H]: 759.1 |
| 106-Ga | 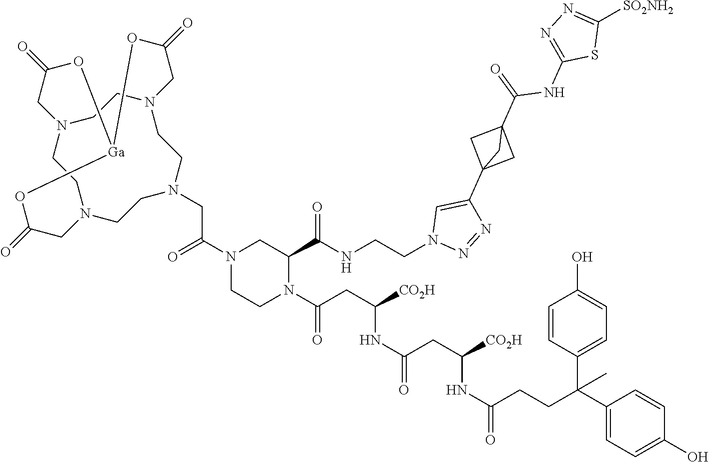 | [M/2 + H]: 724.3 |
| 106-Cu | 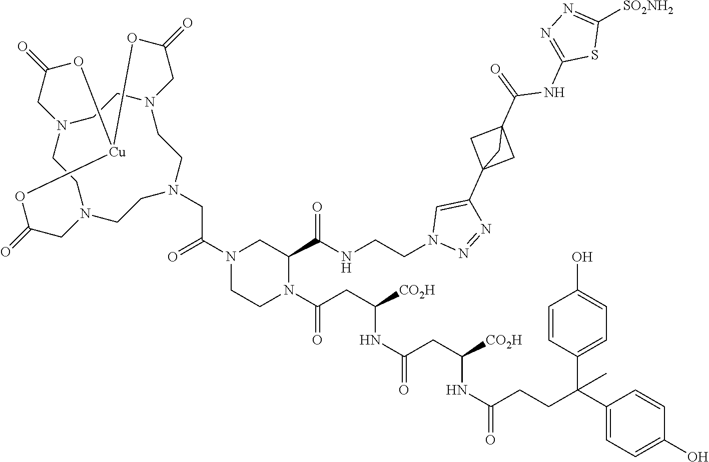 | [M/2 + H]: 721.8 |
| 126-Cu | 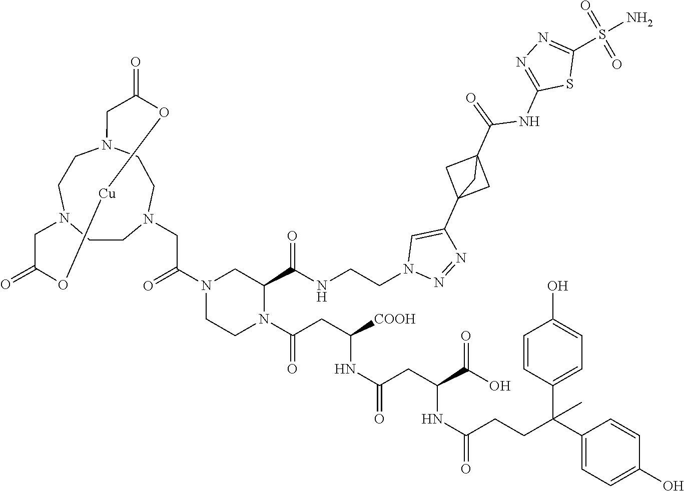 | [M/2 + H]: 671.3 |

### F1. Radiolabeling Protocols

### General Protocol for ¹⁷⁷Lu Radiolabeling of Empty-DOTA Construct

A 35 mCi aliquot of ¹⁷⁷LuCl₃ was added to the reaction vessel followed by addition of 10% ethanol in 0.4M NaOAc buffer (1.0 mL; pH=5.0) and the empty-DOTA construct (1.0 μg/μL, 32 μL). The resulting mixture was heated and mixed with a thermal mixer at 70° C. for 0.5 h. After 0.5 h, the reaction mixture was allowed to cool at room temperature for 3 min. Then the reaction mixture was diluted with 4.8 mL of a pre-mixed formulation buffer. To assess the radiochemical purity, 60 μCi of the 177Lu labeled product was injected on a rad-HPLC. At the end of the synthesis (EOS), the radiochemical purity and specific activity was determined. HPLC column: Aeris 3.6 μm PEPTIDE XB-C18 100, LC Column, 150×4.6 mm (00F-4507-E0). Flow 1.0 mL/min. HPLC column Aeris 3.6 μm PEPTIDE XB-C18 100, LC Column, 150×4.6 mm (00F-4507-E0). Solvent A was H₂O/0.05% TFA and solvent B was CH₃CN/0.05% TFA. Elution was conducted at flow rate of 1.0 mL/min with gradient from 5 to 95% B over 16 min.

### General Protocol for ²²⁵Ac Radiolabeling of Empty-DOTA Construct

A ²²⁵Ac(NO₃)₃ powder was diluted with 0.5M HCl to achieve a concentration of 10 μCi/μL. 100 μCi aliquot of the 10 μCi/μL ²²⁵Ac-chloride solution was added to the reaction vessel followed by addition of 10% ethanol in 0.4M NaOAc buffer (200 μL, pH=6.2) and 27.5 μg of the empty-DOTA construct (1.0 μg/μL, 27.5 μL). The resulting mixture was heated and mixed with a thermal mixer at 90° C. for 0.5 h. After 0.5 h, the reaction mixture was allowed to cool at room temperature for 3 min. The reaction mixture was diluted with 1.0 mL of a pre-mixed formulation buffer. The radiochemical purity was determined at 5 μCi/nmol by radio thin-layer chromatography (radio-TLC). TLC plate: Agilent Technologies Chromatography paper (Cat number A120B12); mobile phase: 50 mM DTPA, pH=5.56.

### General Protocol for ⁶⁴Cu Radiolabeling of Empty-NOTA Construct

⁶⁴CuCl₂ in 0.1M HCl was reconstituted with 0.4M NaOAc pH=6.2 to achieve a concentration of 0.2 mCi/μL. A 7.14 mCi aliquot of the ⁶⁴CuCl₂ solution was added to the reaction vessel followed by addition of 10% ethanol in 0.4M NaOAc buffer (200 μL; pH=6.2) and the empty-NOTA construct (1.0 μg/μL, 6.0 μL). The resulting mixture was heated and mixed with a thermal mixer at 50° C. for 0.5 h. After 0.5 h, the reaction mixture was allowed to cool at room temperature for 3 min. Then the reaction was diluted with 4.8 mL of a pre-mixed formulation buffer. To assess the radiochemical purity, 10 μCi of the product was injected on a rad-HPLC. At the end of the synthesis (EOS), the radiochemical purity and specific activity was determined. HPLC column: Aeris 3.6 μm PEPTIDE XB-C18 100, LC Column, 150×4.6 mm (00F-4507-E0). Solvent A was H₂O/0.05% TFA and solvent B was CH₃CN/0.05% TFA. Elution was conducted at flow rate of 1.0 mL/min with gradient from 5 to 95% B over 16 min.

G1. Surface Plasmon Resonance Assay Conditions for hCA-IX and hCA-XII

Surface Plasmon Resonance (SPR) experiments were conducted on a Biacore 8K instrument (Cytiva, Marlborough, MA) to determine the binding kinetics of the compound to recombinant human CA-IX (Acro, CA9-H82E3) and CA-XII (Creative BioMart, CA12-261H) proteins. The ligands were diluted to 10 μg/mL in PBS-P+ buffer (20 mM phosphate buffer, pH 7.4, with 2.7 mM KCl, 137 mM NaCl, and 0.05% Tween 20) and captured on Series S Streptavidin Sensor Chips (Cytiva, 29699621) to 1200-1800 response units (RU) using a flow rate of 5 μL/min and injection times of 180 seconds for human CA-IX and 75 seconds for human CA-XII. Analyte binding was evaluated using a single-cycle kinetics method over 9 analyte concentrations (100-0.39 nM) in PBS-P+ buffer with 1% DMSO using a 50 μL/min. flow rate, 120 second association time, and 1200 second dissociation time. The flow cell temperature was set to 25° C. Analyte binding curves were fit to a 1:1 binding kinetic model in Biacore Insight Evaluation software (version 4.0.8.20368) following reference flow cell subtraction and DMSO correction to derive the association and dissociation binding constants. The data is presented in table 11.

TABLE 11

| Conjugates Tested | | | | |
|---|---|---|---|---|
| Comprising a | | | | |
| compound and, | | | | |
| when indicated, a | | | | |
| chelated metal | | | | Ratio |
| | Chelated | CAIX | CAXII | (CAXII/ |
| Compound | Metal | KD | KD | CAIX) |
| 100 | Lu-175 | B | D | *** |
| 100.1 | Lu-175 | A | B | * |
| 100.2 | Lu-175 | B | D | *** |
| 100.3 | Lu-175 | A | C | ** |
| 100.4 | Lu-175 | A | C | * |
| 100.5 | Lu-175 | A | A | * |
| 100.6 | Lu-175 | A | B | ** |
| 100.7 | Lu-175 | A | B | * |
| 100.8 | Lu-175 | A | C | * |
| 100.9 | Lu-175 | A | B | * |
| 100.10 | Lu-175 | A | A | * |
| 100.11 | Lu-175 | A | A | * |
| 100.12 | Lu-175 | A | A | * |
| 100.13 | Lu-175 | A | A | * |
| 100.14 | Lu-175 | A | A | * |
| 100.15 | Lu-175 | A | A | * |
| 100.16 | Lu-175 | A | A | * |
| 100.17 | Lu-175 | A | A | * |
| 100.18 | Lu-175 | A | A | * |
| 100.19 | Lu-175 | A | A | * |
| 101 | Lu-175 | A | A | * |
| 101.1 | Lu-175 | A | C | ** |
| 101.2 | Lu-175 | A | C | *** |
| 102 | Lu-175 | B | D | ** |
| 102.1 | Lu-175 | A | D | *** |
| 102.2 | Lu-175 | C | D | * |
| 102.3 | None | A | D | *** |
| 102.4 | Lu-175 | A | C | ** |
| 102.5 | Lu-175 | A | C | *** |
| 102.6 | Lu-175 | A | D | *** |
| 102.7 | Lu-175 | A | C | *** |
| 102.8 | Lu-175 | A | B | * |
| 103 | Lu-175 | B | C | * |
| 103.1 | Lu-175 | B | C | * |
| 103.2 | Lu-175 | B | C | * |
| 103.3 | Lu-175 | C | D | * |
| 103.4 | Lu-175 | C | C | * |
| 104 | Lu-175 | B | C | * |
| 104.1 | None | A | A | * |
| 104.2 | None | A | A | * |
| 106 | Lu-175 | A | C | *** |
| 107 | Lu-175 | A | D | **** |
| 107.1 | Lu-175 | A | A | * |
| 107.2 | Lu-175 | A | A | * |
| 107.3 | Lu-175 | B | D | *** |
| 107.4 | Lu-175 | B | D | *** |
| 107.5 | Lu-175 | A | A | * |
| 107.6 | Lu-175 | A | D | **** |
| 107.7 | Lu-175 | A | B | * |
| 107.8 | Lu-175 | B | D | *** |
| 107.9 | Lu-175 | A | B | * |
| 107.10 | Lu-175 | A | A | * |
| 107.11 | Lu-175 | A | D | *** |
| 107.12 | Lu-175 | B | D | **** |
| 107.13 | Lu-175 | B | D | *** |
| 107.14 | Lu-175 | A | A | * |
| 107.15 | Lu-175 | B | D | *** |
| 107.16 | Lu-175 | B | C | ** |
| 107.17 | Lu-175 | A | B | * |
| 107.18 | Lu-175 | C | D | *** |
| 107.19 | Lu-175 | B | D | **** |
| 107.20 | Lu-175 | ND | ND | |
| 107.21 | Lu-175 | B | D | *** |
| 107.22 | Lu-175 | B | D | *** |
| 107.23 | Lu-175 | A | D | *** |
| 107.24 | Lu-175 | B | C | * |
| 107.25 | Lu-175 | A | D | **** |
| 107.26 | Lu-175 | A | D | *** |
| 107.27 | Lu-175 | A | A | *** |
| 107.28 | Lu-175 | A | A | *** |
| 107.29 | Lu-175 | A | D | **** |
| 107.30 | Lu-175 | A | B | ** |
| 107.31 | Lu-175 | B | D | **** |
| 107.32 | Lu-175 | A | D | **** |
| 107.33 | Lu-175 | A | D | **** |
| 107.34 | Lu-175 | A | D | **** |
| 107.35 | Lu-175 | A | D | *** |
| 107.36 | Lu-175 | A | D | *** |
| 107.37 | Lu-175 | D | D | *** |
| 107.38 | Lu-175 | A | D | **** |
| 107.39 | Lu-175 | A | D | *** |
| 107.40 | Lu-175 | ND | A | |
| 107.41 | Lu-175 | B | D | *** |
| 107.42 | Lu-175 | A | C | * |
| 107.43 | Lu-175 | C | D | **** |
| 107.44 | Lu-175 | A | D | **** |
| 107.45 | Lu-175 | A | C | * |
| 107.46 | Lu-175 | D | ND | |
| 107.47 | Lu-175 | B | D | **** |
| 107.48 | Lu-175 | A | B | **** |
| 107.49 | Lu-175 | A | D | **** |
| 107.50 | Lu-175 | A | D | *** |
| 107.51 | Lu-175 | B | D | **** |
| 107.52 | Lu-175 | B | D | **** |
| 107.53 | None | A | C | **** |
| 107.54 | Lu-175 | C | D | * |
| 107.55 | Lu-175 | A | C | *** |
| 107.56 | Lu-175 | B | D | **** |
| 107.57 | Lu-175 | B | D | **** |
| 107.58 | Lu-175 | A | D | **** |
| 107.59 | Lu-175 | A | D | **** |
| 107.60 | Lu-175 | A | C | ** |
| 107.61 | Lu-175 | B | D | ** |
| 108 | Lu-175 | B | D | *** |
| 108.1 | Lu-175 | C | D | *** |
| 108.2 | Lu-175 | A | D | **** |
| 108.3 | Lu-175 | C | D | **** |
| 108.4 | Lu-175 | B | D | *** |
| 108.5 | Lu-175 | C | D | *** |
| 109 | Lu-175 | A | C | ** |
| 109.1 | Lu-175 | A | C | * |
| 109.2 | Lu-175 | A | B | ** |
| 109.3 | Lu-175 | A | A | * |
| 109.4 | Lu-175 | A | C | ** |
| 109.5 | Lu-175 | B | C | ** |
| 109.6 | Lu-175 | A | C | ** |
| 109.7 | Lu-175 | A | C | * |
| 109.8 | Lu-175 | B | C | * |
| 109.9 | Lu-175 | A | B | * |
| 109.10 | Lu-175 | A | C | *** |
| 109.11 | Lu-175 | A | C | *** |
| 109.12 | Lu-175 | B | C | * |
| 109.13 | Lu-175 | A | C | *** |
| 109.14 | Lu-175 | A | C | *** |
| 109.15 | Lu-175 | A | C | ** |
| 109.16 | Lu-175 | A | C | ** |
| 109.17 | Lu-175 | C | D | * |
| 109.18 | Lu-175 | A | B | * |
| 109.19 | Lu-175 | A | B | * |
| 109.20 | Lu-175 | A | C | ** |
| 109.21 | Lu-175 | A | D | *** |
| 109.22 | Lu-175 | B | C | * |
| 109.23 | Lu-175 | C | D | * |
| 109.24 | Lu-175 | B | C | * |
| 109.25 | Lu-175 | A | C | *** |
| 109.26 | Lu-175 | B | C | * |
| 109.27 | Lu-175 | B | C | * |
| 109.28 | Lu-175 | A | B | * |
| 109.29 | Lu-175 | C | D | *** |
| 109.30 | Lu-175 | A | C | ** |
| 109.31 | Lu-175 | A | C | ** |
| 109.32 | Lu-175 | A | D | *** |
| 109.33 | Lu-175 | B | D | *** |
| 109.34 | Lu-175 | A | C | ** |
| 109.35 | Lu-175 | B | C | * |
| 109.36 | Lu-175 | B | C | ** |
| 109.37 | Lu-175 | A | C | *** |
| 109.38 | Lu-175 | A | C | ** |
| 109.39 | Lu-175 | B | C | * |
| 109.40 | Lu-175 | A | D | **** |
| 109.41 | Lu-175 | A | A | * |
| 109.42 | Lu-175 | A | A | * |
| 109.43 | Lu-175 | A | A | * |
| 109.44 | Lu-175 | A | A | * |
| 109.45 | Lu-175 | A | A | * |
| 109.46 | Lu-175 | A | A | * |
| 109.47 | Lu-175 | A | A | * |
| 109.48 | Lu-175 | A | A | * |
| 109.49 | Lu-175 | A | A | * |
| 109.50 | Lu-175 | A | A | * |
| 109.51 | Lu-175 | A | A | * |
| 109.52 | Lu-175 | A | A | * |
| 109.53 | Lu-175 | A | A | * |
| 109.54 | Lu-175 | A | A | ** |
| 109.55 | Lu-175 | A | A | * |
| 109.56 | Lu-175 | A | B | * |
| 109.57 | Lu-175 | A | A | * |
| 109.58 | Lu-175 | A | A | *** |
| 109.59 | Lu-175 | A | A | * |
| 109.60 | Lu-175 | A | B | *** |
| 109.61 | Lu-175 | B | C | * |
| 109.62 | None | A | D | *** |
| 109.63 | None | A | C | *** |
| 110 | Lu-175 | A | A | * |
| 111 | Lu-175 | C | D | *** |
| 111.1 | Lu-175 | B | D | *** |
| 111.2 | Lu-175 | C | D | *** |
| 111.3 | Lu-175 | B | D | *** |
| 111.4 | Lu-175 | C | D | *** |
| 111.5 | Lu-175 | C | D | *** |
| 111.6 | Lu-175 | C | D | *** |
| 111.7 | Lu-175 | C | D | *** |
| 111.8 | Lu-175 | C | D | *** |
| 111.9 | Lu-175 | B | D | *** |
| 111.10 | Lu-175 | C | D | *** |
| 111.11 | Lu-175 | B | D | *** |
| 111.12 | Lu-175 | C | D | *** |
| 112 | Lu-175 | C | D | *** |
| 112.1 | Lu-175 | C | D | *** |
| 112.2 | Lu-175 | A | C | * |
| 112.3 | Lu-175 | B | D | *** |
| 112.4 | Lu-175 | C | D | *** |
| 112.5 | Lu-175 | C | D | *** |
| 112.6 | Lu-175 | B | D | *** |
| 112.7 | Lu-175 | A | D | *** |
| 112.8 | Lu-175 | A | C | *** |
| 112.9 | Lu-175 | A | D | **** |
| 112.10 | Lu-175 | A | D | *** |
| 112.11 | Lu-175 | C | D | **** |
| 112.12 | Lu-175 | B | D | *** |
| 112.13 | Lu-175 | B | D | **** |
| 112.14 | Lu-175 | C | D | *** |
| 112.15 | Lu-175 | B | D | *** |
| 112.16 | Lu-175 | C | D | *** |
| 112.17 | Lu-175 | A | B | * |
| 112.18 | Lu-175 | B | D | **** |
| 112.19 | Lu-175 | A | D | *** |
| 113 | Lu-175 | C | D | **** |
| 114 | Lu-175 | B | D | *** |
| 114.1 | Lu-175 | B | D | *** |
| 114.2 | Lu-175 | B | D | *** |
| 114.3 | Lu-175 | B | D | *** |
| 115 | Lu-175 | C | D | *** |
| 116 | Lu-175 | B | C | * |
| 116.1 | Lu-175 | B | C | * |
| 116.2 | Lu-175 | B | C | * |
| 116.3 | Lu-175 | B | C | * |
| 116.4 | Lu-175 | C | D | ** |
| 117 | Lu-175 | B | D | *** |
| 117.1 | Lu-175 | A | D | **** |
| 117.2 | None | ND | ND | |
| 117.3 | Lu-175 | A | D | **** |
| 117.4 | Lu-175 | B | D | **** |
| 117.5 | Lu-175 | A | C | **** |
| 117.6 | Lu-175 | A | B | **** |
| 117.7 | Lu-175 | A | D | **** |
| 117.8 | Lu-175 | A | D | **** |
| 118 | Lu-175 | B | C | ** |
| 118.1 | Lu-175 | B | D | *** |
| 118.2 | Lu-175 | B | D | *** |
| 118.3 | Lu-175 | C | C | * |
| 118.4 | Lu-175 | B | D | *** |
| 119 | Lu-175 | C | D | ** |
| 120 | None | ND | ND | |
| 121 | Lu-175 | A | D | **** |
| 121.1 | Lu-175 | B | D | **** |
| 122 | Lu-175 | B | B | * |
| 123 | Lu-175 | B | C | * |
| 124 | Lu-175 | C | C | * |
| 125 | Lu-175 | B | D | ** |
| 125.1 | Lu-175 | C | D | *** |
| 126 | Cu (cold) | A | D | **** |
| 126.1 | Cu (cold) | A | D | **** |
| 126.2 | Cu (cold) | A | C | *** |
| 126.3 | Cu (cold) | A | D | *** |
| 126.4 | Cu (cold) | A | D | *** |
| 126.5 | Cu (cold) | A | B | *** |
| 127 | Lu-175 | A | D | *** |
| 127.1 | Lu-175 | A | D | **** |
| 127.2 | Lu-175 | A | D | **** |
| 127.3 | Cu (cold) | A | D | **** |
| 127.4 | Lu-175 | A | D | **** |
| 127.5 | Lu-175 | A | C | **** |
| 127.6 | Lu-175 | A | A | **** |
| 127.7 | Lu-175 | A | D | **** |
| 127.8 | Lu-175 | A | D | **** |
| 127.9 | Lu-175 | A | D | **** |
| 128 | Lu-175 | A | D | *** |
| 128.1 | Lu-175 | A | D | **** |
| 129 | Lu-175 | B | D | *** |
| 129.1 | Lu-175 | A | D | **** |
| 129.2 | Lu-175 | A | D | **** |
| 129.3 | Lu-175 | A | D | **** |
| 129.4 | None | A | D | **** |
| 129.5 | Lu-175 | A | D | **** |
| 129.6 | Lu-175 | A | D | **** |
| 129.7 | Lu-175 | A | D | **** |
| 130 | None | A | C | *** |
| 131 | Lu-175 | A | C | **** |
| 131.1 | None | A | A | * |
| 131.2 | None | A | A | ** |
| 131.3 | Lu-175 | A | A | **** |
| 131.4 | None | A | A | * |
| 131.5 | Lu-175 | A | A | * |
| 131.6 | Lu-175 | A | B | **** |
| 131.7 | Lu-175 | A | B | **** |
| 132 | Lu-175 | A | B | * |
| 133 | Lu-175 | A | D | **** |
| 134 | Lu-175 | A | D | **** |
| 135 | Lu-175 | A | D | **** |
| 136 | Lu-175 | A | D | **** |

| CAIX KD | CAXII KD | Ratio |
|---|---|---|
| (nM) | (nM) | (CAXII/CAIX |
| 0 < A ≤ 1 | 0 < A ≤ 1 | 1 < * < 5 |
| 1 < B ≤ 2.5 | 1 < B ≤ 2.5 | 5 < ** < 10 |
| 2.5 < C ≤ 10 | 2.5 < C ≤ 10 | 10 < *** ≤ 50 |
| 10 < D | 10 < D | 50 < **** |
| ND: | ND: | |
| not determined | not determined | |

What is claimed:

1. A conjugate of Formula (A), or a pharmaceutically acceptable salt or stereoisomer thereof: 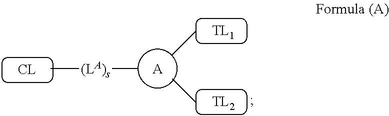
wherein:
TL₁ represents a 1^{st} targeting moiety that binds to carbonic anhydrase IX;
TL₂ represents a 2^{nd} targeting moiety that binds to carbonic anhydrase IX;
L^{A} represents a linker;
s is 0 or 1;
Ring A is an optionally substituted cycloalkyl, optionally substituted heterocycloalkyl, optionally substituted aryl, or optionally substituted heteroaryl; and
CL represents a metal chelator.

2. A conjugate of Formula (A′), or a pharmaceutically acceptable salt or stereoisomer thereof: 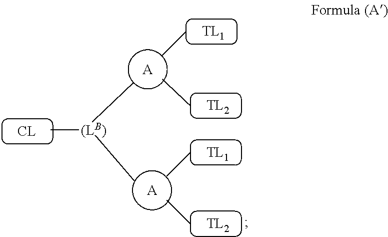
wherein:
each TL₁ independently represents a 1^{st} targeting moiety that binds to carbonic anhydrase IX;
each TL₂ independently represents a 2^{nd} targeting moiety that binds to carbonic anhydrase IX;
L^{B} represents a linker; and
each Ring A is independently an optionally substituted cycloalkyl, optionally substituted heterocycloalkyl, optionally substituted aryl, or optionally substituted heteroaryl; and
CL represents a metal chelator.

3. The conjugate of claim 1 or 2, wherein:
Ring A is cycloalkyl, heterocycloalkyl, aryl, or heteroaryl, wherein the cycloalkyl, heterocycloalkyl, aryl, and heteroaryl is optionally substituted with one or more R; and
each R is independently halogen, —CN, —OH, —SF₅, —SH, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, C₃-C₆cycloalkyl, or 3- to 6-membered heterocycloalkyl;
or two R on the same atom form an oxo.

4. The conjugate of any one of claims 1-3, wherein

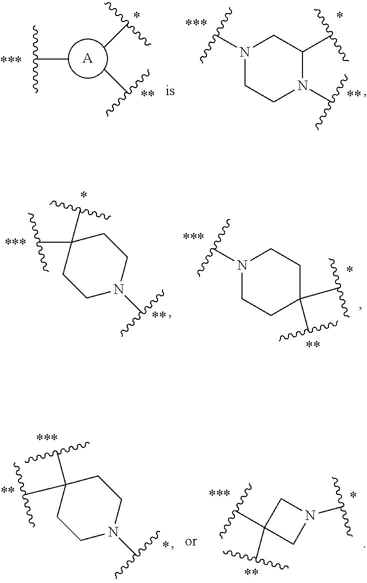

5. The conjugate of any one of claims 1-3, wherein

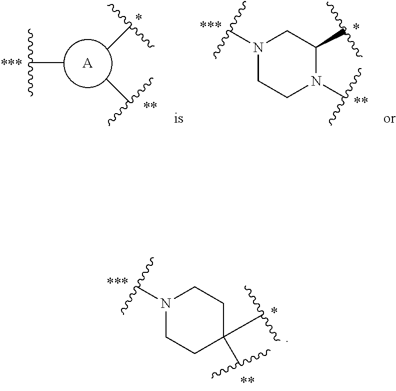

6. The conjugate of any one of claims 1-5, wherein TL₁ is of Formula (IA): 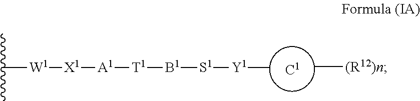
wherein:
W¹ is absent, —O—, —S—, —S(═O)—, —S(═O)₂—, —NR¹¹—, —C(═O)—, —C(═O)NR¹¹—, —NR¹¹C(═O)—, —S(═O)₂NR¹¹—, or —NR¹¹S(═O)₂;
X¹ is C₁-C₁₀alkylene optionally substituted with one or more R and wherein one, two, three, or four carbon atoms of the C₁-C₁₀alkylene are optionally replaced with —S—, —S(═O)—, —S(═O)₂—, —O—, or —NR¹¹—;
A¹ is absent, cycloalkyl, heterocycloalkyl, aryl, or heteroaryl, wherein the cycloalkyl, heterocycloalkyl, aryl, and heteroaryl is optionally substitued with one or more R;
T¹ is absent or C₁-C₃alkylene optionally substituted with one or more R and wherein one carbon atom of the C₁-C₃alkylene is optionally replaced with —C(═O)—, —S—, —S(═O)—, —S(═O)₂—, —O—, or —NR¹¹—;
B¹ is absent, C₁-C₁₀alkylene, cycloalkyl, heterocycloalkyl, aryl, or heteroaryl, wherein the alkylene, cycloalkyl, heterocycloalkyl, aryl, and heteroaryl is optionally substitued with one or more R, and wherein one, two, three, or four carbon atoms in the C₁-C₁₀alkylene are optionally replaced with —S—, —S(═O)—, —S(═O)₂—, —O—, —NR¹¹—;
S¹ is absent or C₁-C₃alkylene optionally substituted with one or more R and wherein one or two carbon atoms of the C₁-C₃alkylene are optionally replaced with —S—, —S(═O)—, —S(═O)₂—, —O—, or —NR¹¹—;
Y¹ is absent, —O—, —S—, —S(═O)—, —S(═O)₂—, —NR¹¹—, —C(═O)—, —C(═O)NR¹¹—, —NR¹¹C(═O)—, —S(═O)₂NR¹¹—, or —NR¹¹S(═O)₂;
R¹¹ is hydrogen, C₁-C₆alkyl, C₁-C₆haloalkyl, C₁-C₆hydroxyalkyl, C₁-C₆aminoalkyl, or C₁-C₆heteroalkyl;
Ring C¹ is cycloalkyl, heterocycloalkyl, aryl, or heteroaryl;
each R¹² is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
n is 0, 1, 2, 3, 4, 5, or 6;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, -L-cycloalkyl, -L-heterocycloalkyl, -L-aryl, or -L-heteroaryl, wherein each alkyl, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, -L-cycloalkyl, -L-heterocycloalkyl, -L-aryl, or -L-heteroaryl, wherein each alkyl, alkenyl, alkynyl, cycloalkyl, heterocycloalkyl, aryl, and heteroaryl is independently optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, -L-cycloalkyl, -L-heterocycloalkyl, -L-aryl, or -L-heteroaryl, wherein each alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
L is absent or C₁-C₃alkylene optionally substituted with one or more R; and
each R is independently halogen, —CN, —OH, —SF₅, —SH, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, C₃-C₆cycloalkyl, or 3- to 6-membered heterocycloalkyl;
or two R on the same atom form an oxo;
or two R on the same or different atoms are taken together to form a cycloalkyl, heterocycloalkyl, aryl, or heteroaryl.

7. The conjugate of claim 6, wherein W¹ is absent, —C(═O)—, or —C(═O)NR¹¹—.

8. The conjugate of claim 6 or 7, wherein X¹ is C₁-C₄alkylene.

9. The conjugate of claim 6, wherein

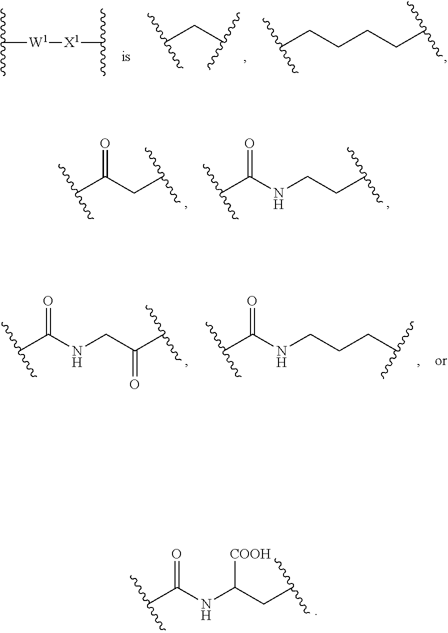

10. The conjugate of claim 6 or 9, wherein

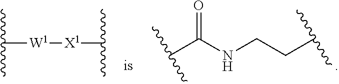

11. The conjugate of any one of claims 6-10, wherein A¹ is 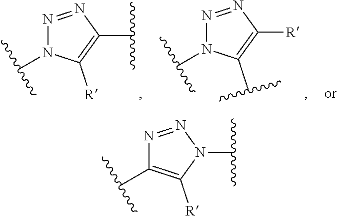
wherein R′ is hydrogen or R.

12. The conjugate of any one of claims 6-11, wherein T¹ is absent or C₁-C₃alkylene.

13. The conjugate of any one of claims 6-12, wherein B¹ is C₁-C₆alkylene.

14. The conjugate of any one of claims 6-12, wherein B¹ is cycloalkyl optionally substitued with one or more R.

15. The conjugate of any one of claims 6-12, wherein B¹ is a bicyclic cycloalkyl optionally substitued with one or more R.

16. The conjugate of any one of claims 6-12, wherein B¹ is bicyclo[1.1.1]pentanyl.

17. The conjugate of any one of claims 6-16, wherein S¹ is absent.

18. The conjugate of any one of claims 12-17 wherein

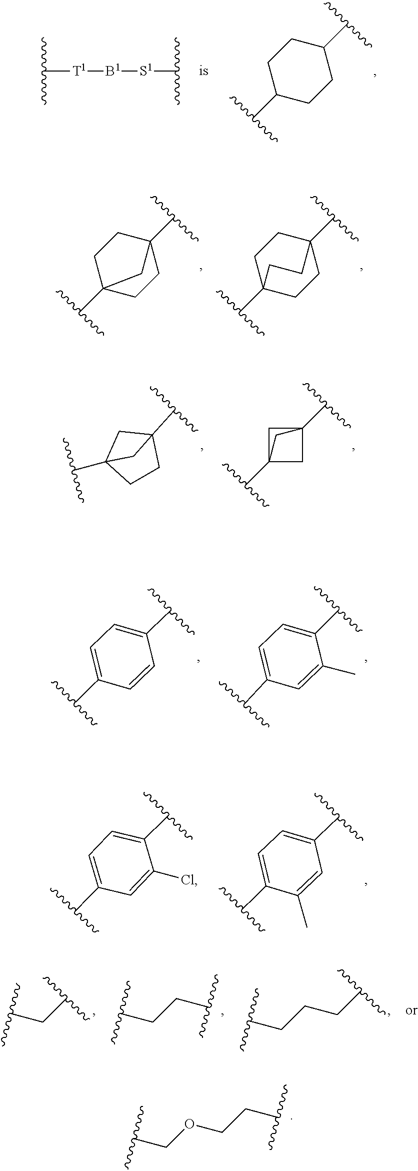

19. The conjugate of any one of claims 12-18 wherein

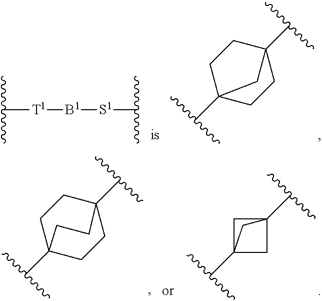

20. The conjugate of any one of claims 12-19, wherein

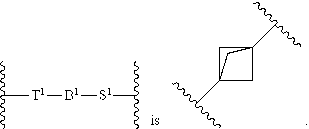

21. The conjugate of any one of claims 6-20, wherein Y¹ is absent, —O—, —S—, —S(═O)₂—, —C(═O)NR¹¹—, or —NR¹¹C(═O)—.

22. The conjugate of any one of claims 6-21, wherein Ring C¹ is heteroaryl or phenyl.

23. The conjugate of any one of claims 6-21, wherein Ring C¹ is monocyclic heteroaryl.

24. The conjugate of any one of claims 6-21, wherein Ring C¹ is bicyclic heteroaryl.

25. The conjugate of any one of claims 6-21, wherein 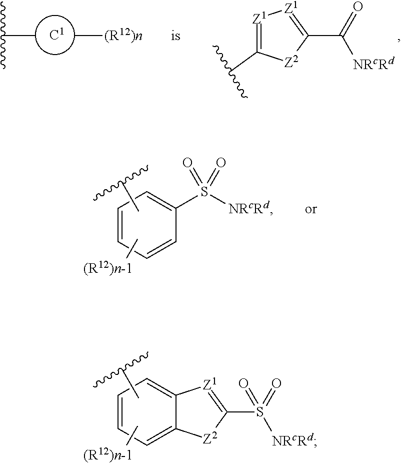
wherein each Z¹ is independently —N— or —CR¹²′—;
Z² is —O—, —S—, or —NR¹²″—;
each R¹²′ is independently hydrogen, halogen, —CN, —OH, —OR^{a}, —NR^{c}R^{d}, C₁-C₆alkyl, C₁-C₆haloalkyl, cycloalkyl, or heterocycloalkyl; and
R¹²″ is hydrogen, —S(═O)R^{a}, —S(═O)₂R^{a}, —S(═O)₂NR^{c}R^{d}, —C(═O)R^{a}, —C(═O)OR^{b}, —C(═O)NR^{c}R^{d}, C₁-C₆alkyl, C₁-C₆haloalkyl, cycloalkyl, or heterocycloalkyl.

26. The conjugate of any one of claims 6-21, wherein

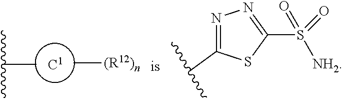

27. The conjugate of any one of claims 6-21, wherein

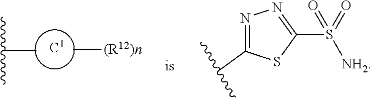

28. The conjugate of any one of claims 6-21, wherein

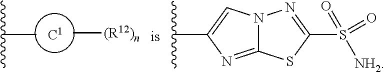

29. The conjugate of any one of claims 1-28, wherein TL₂ is of Formula (IIA): 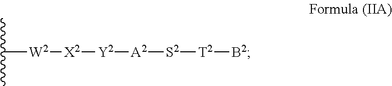
wherein:
W² is absent, —O—, —S—, —S(═O)—, —S(═O)₂—, —NR²¹—, —C(═O)—, —C(═O)NR²¹—, —NR²¹C(═O)—, —S(═O)₂NR²¹—, or —NR²¹S(═O)₂;
X² is absent or C₁-C₁₀alkylene optionally substituted with one or more R, and wherein one, two, three, or four carbon atoms of the C₁-C₁₀alkylene are optionally replaced with —S—, —S(═O)—, —S(═O)₂—, 0-O—, or —NR²¹—;
Y² is absent, —O—, —S—, —S(═O)—, —S(═O)₂—, —NR²¹—, —C(═O)—, —C(═O)NR²¹—, —NR²¹C(═O)—, —S(═O)₂NR²¹—, or —NR²¹S(═O)₂;
A² is absent, C₁-C₁₀alkylene, cycloalkyl, heterocycloalkyl, aryl, or heteroaryl, wherein the alkylene, cycloalkyl, heterocycloalkyl, aryl, and heteroaryl is optionally substitued with one or more R²²;
each R²² is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
S² is absent, —O—, —S—, —S(═O)—, —S(═O)₂—, —NR²¹—, —C(═O)—, —C(═O)NR²¹—, —NR²¹C(═O)—, —S(═O)₂NR²¹—, or —NR²¹S(═O)₂;
T² is absent or C₁-C₁₀alkylene optionally substituted with one or more R, and wherein one, two, three, or four carbon atoms of the C₁-C₁₀alkylene are optionally replaced with —S—, —S(═O)—, —S(═O)₂—, —O—, or —NR²¹—;
R²¹ is hydrogen, C₁-C₆alkyl, C₁-C₆haloalkyl, C₁-C₆hydroxyalkyl, C₁-C₆aminoalkyl, or C₁-C₆heteroalkyl;
(i) B² is absent;
or
(ii) B² is —N(R²³)₂;
each R²³ is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
or
(iii) B² is —CF₃;
or
(iv) B² is cycloalkyl, heterocycloalkyl, aryl, or heteroaryl; wherein the cycloalkyl, heterocycloalkyl, aryl, and heteroaryl is optionally substitued with one or more R²⁴;
each R²⁴ is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
or
(v) B² is 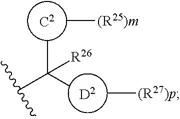
Ring C² is cycloalkyl, heterocycloalkyl, aryl, or heteroaryl;
each R²⁵ is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
m is 0, 1, 2, 3, or 4;
R²⁶ is hydrogen, C₁-C₆alkyl, C₁-C₆haloalkyl, C₁-C₆hydroxyalkyl, C₁-C₆aminoalkyl, or C₁-C₆heteroalkyl;
Ring D² is cycloalkyl, heterocycloalkyl, aryl, or heteroaryl;
each R²⁷ is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
p is 0, 1, 2, 3, or 4;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, -L-cycloalkyl, -L-heterocycloalkyl, -L-aryl, or -L-heteroaryl, wherein each alkyl, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, -L-cycloalkyl, -L-heterocycloalkyl, -L-aryl, or -L-heteroaryl, wherein each alkyl, alkenyl, alkynyl, cycloalkyl, heterocycloalkyl, aryl, and heteroaryl is independently optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, -L-cycloalkyl, -L-heterocycloalkyl, -L-aryl, or -L-heteroaryl, wherein each alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
L is absent or C₁-C₃alkylene optionally substituted with one or more R; and
each R is independently halogen, —CN, —OH, —SF₅, —SH, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, C₃-C₆cycloalkyl, or 3- to 6-membered heterocycloalkyl;
or two R on the same atom form an oxo;
or two R on the same or different atoms are taken together to form a cycloalkyl, heterocycloalkyl, aryl, or heteroaryl.

30. The conjugate of claim 29, wherein W² is absent, —C(═O)—, or —NR²¹C(═O)—.

31. The conjugate of claim 29 or 30, wherein X² is C₁-C₄alkylene optionally substituted with one or more R.

32. The conjugate of any one of claims 29-31, wherein X² is —CH₂—.

33. The conjugate of any one of claims 29-32, wherein Y² is absent, —NR²¹—, or —NR²¹C(═O)—.

34. The conjugate of any one of claims 29-33, wherein Y² is —NR²¹C(═O)—.

35. The conjugate of claim 29, wherein

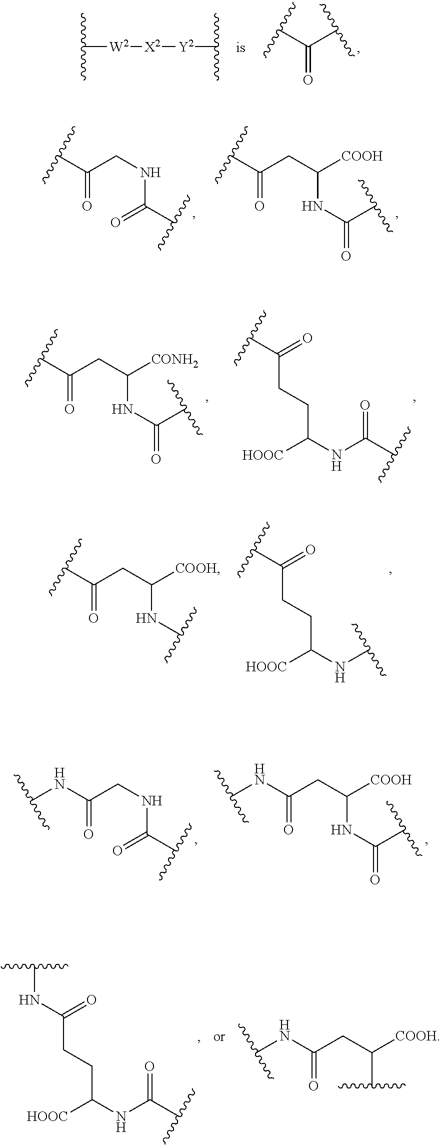

36. The conjugate of claim 29, wherein

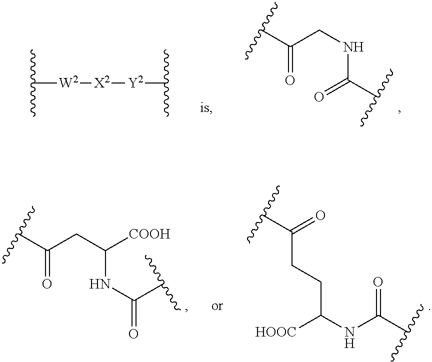

37. The conjugate of any one of claims 29-36, wherein A² is C₁-C₆alkylene optionally substitued with one or more R²².

38. The conjugate of any one of claims 29-36, wherein A² is heterocycloalkyl optionally substitued with one or more R²².

39. The conjugate of any one of claims 29-36, wherein A² is pyrrolidinyl, piperidinyl, piperazinyl, or morpholinyl, each optionally substitued with one or more R²².

40. The conjugate of any one of claims 29-36, wherein A² is piperidinyl optionally substitued with one or more R²².

41. The conjugate of any one of claims 29-36, wherein A² is 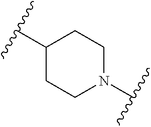 [SMILES: *C1CCN(*)CC1]
optionally substitued with one or more R²².

42. The conjugate of any one of claims 29-36, wherein A² is

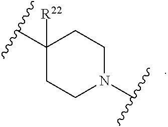

43. The conjugate of any one of claims 29-42, wherein each R²² is independently —C(═O)OR^{b} or —C(═O)NR^{c}R^{d}.

44. The conjugate of any one of claims 29-43, wherein each R²² is independently —C(═O)NR^{c}R^{d}.

45. The conjugate of any one of claims 29-44, wherein S² is absent, —NR²¹—, —C(═O)—, or —NR²¹C(═O)—.

46. The conjugate of any one of claims 29-45, wherein T² is absent or C₁-C₆alkylene optionally substituted with one or more R.

47. The conjugate of claim 45 or 46, wherein

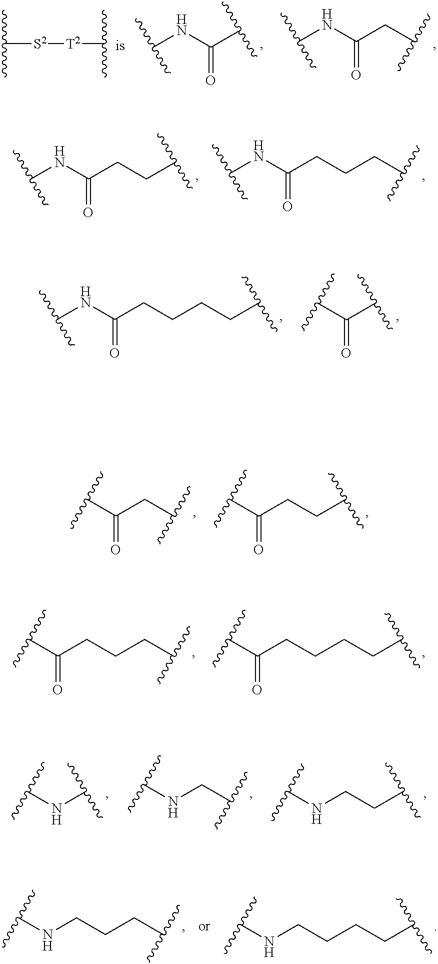

48. The conjugate of any one of claims 45-47, wherein

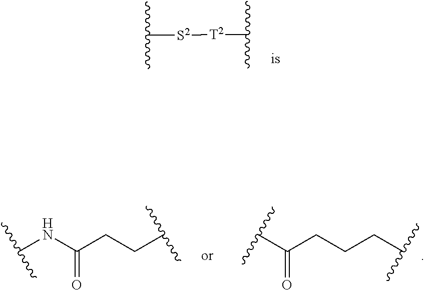

49. The conjugate of any one of claims 29-48, wherein B² is cycloalkyl, heterocycloalkyl, aryl, or heteroaryl; wherein the cycloalkyl, heterocycloalkyl, aryl, and heteroaryl is optionally substitued with one or more R²⁴.

50. The conjugate of any one of claims 29-49, wherein each R²⁴ is independently halogen, —OH, C₁-C₆alkyl, or C₁-C₆haloalkyl.

51. The conjugate of any one of claims 29-48, wherein B² is 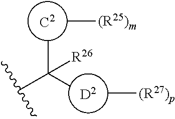
wherein:
Ring C² is cycloalkyl, heterocycloalkyl, aryl, or heteroaryl;
each R²⁵ is independently halogen, —CN, —NO₂, —OH, —ORₐ, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
m is 0, 1, 2, 3, or 4;
R²⁶ is hydrogen, C₁-C₆alkyl, C₁-C₆haloalkyl, C₁-C₆hydroxyalkyl, C₁-C₆aminoalkyl, or C₁-C₆heteroalkyl;
Ring D² is cycloalkyl, heterocycloalkyl, aryl, or heteroaryl;
each R²⁷ is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R; and
p is 0, 1, 2, 3, or 4.

52. The conjugate of any one of claims 29-48, wherein B² is

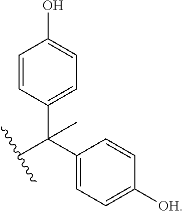 [SMILES: *C(C)(c1ccc(O)cc1)c1ccc(O)cc1]

53. A conjugate of Formula (B), or a pharmaceutically acceptable salt or stereoisomer thereof: 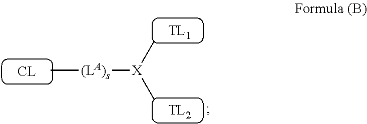
wherein:
L^{A} represents a linker;
s is 0 or 1;
CL represents a metal chelator;
X is N, optionally substituted CH, optionally substituted cycloalkyl, optionally substituted heterocycloalkyl, optionally substituted aryl, or optionally substituted heteroaryl;
TL₁ is of Formula (IB): 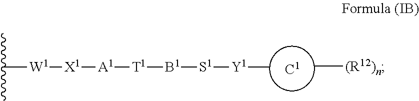
wherein:
W¹ is absent, —O—, —S—, —S(═O)—, —S(═O)₂—, —NR¹¹—, —C(═O)—, —C(═O)NR¹¹—, —NR¹¹C(═O)—, —S(═O)₂NR¹¹—, or —NR¹¹S(═O)₂;
X¹ is C₁-C₁₀alkylene optionally substituted with one or more R and wherein one, two, three, or four carbon atoms of the C₁-C₁₀alkylene are optionally replaced with —S—, —S(═O)—, —S(═O)₂—, —O—, or —NR¹¹—;
A¹ is absent, cycloalkyl, heterocycloalkyl, aryl, or heteroaryl, wherein the cycloalkyl, heterocycloalkyl, aryl, and heteroaryl is optionally substitued with one or more R;
T¹ is absent or C₁-C₃alkylene optionally substituted with one or more R and wherein one carbon atom of the C₁-C₃alkylene is optionally replaced with —C(═O)—, —S—, —S(═O)—, —S(═O)₂—, —O—, —NR¹¹—;
B¹ is cycloalkyl, heterocycloalkyl, or aryl, wherein the cycloalkyl, heterocycloalkyl, and aryl is optionally substitued with one or more R; or
B¹ is heteroaryl substituted with one or more R;
S¹ is absent or C₁-C₃alkylene optionally substituted with one or more R and wherein one or two carbon atoms of the C₁-C₃alkylene are optionally replaced with —C(═O)—, —S—, —S(═O)—, —S(═O)₂—, —O—, or —NR¹¹—;
Y¹ is absent, —O—, —S—, —S(═O)—, —S(═O)₂—, —NR¹¹—, —C(═O)—, —C(═O)NR¹¹—, —NR¹¹C(═O)—, —S(═O)₂NR¹¹—, or —NR¹¹S(═O)₂;
R¹¹ is hydrogen, C₁-C₆alkyl, C₁-C₆haloalkyl, C₁-C₆hydroxyalkyl, C₁-C₆aminoalkyl, or C₁-C₆heteroalkyl;
Ring C¹ is cycloalkyl, heterocycloalkyl, aryl, or heteroaryl;
each R¹² is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
n is 0, 1, 2, 3, 4, 5, or 6;
TL₂ is of Formula (IIB): 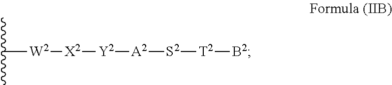
wherein:
W² is absent, —O—, —S—, —S(═O)—, —S(═O)₂—, —NR²¹—, —C(═O)—, —C(═O)NR²¹—, —NR²¹C(═O)—, —S(═O)₂NR²¹—, or —NR²¹S(═O)₂;
X² is absent or C₁-C₁₀alkylene optionally substituted with one or more R, and wherein one, two, three, or four carbon atoms of the C₁-C₁₀alkylene are optionally replaced with —S—, —S(═O)—, —S(═O)₂—, —O—, or —NR²¹—;
Y² is absent, —O—, —S—, —S(═O)—, —S(═O)₂—, —NR²¹—, —C(═O)—, —C(═O)NR²¹—, —NR²¹C(═O)—, —S(═O)₂NR²¹—, or —NR²¹S(═O)₂;
A² is absent, C₁-C₁₀alkylene, cycloalkyl, heterocycloalkyl, aryl, or heteroaryl, wherein the alkylene, cycloalkyl, heterocycloalkyl, aryl, and heteroaryl is optionally substitued with one or more R²²;
each R²² is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
S² is absent, —O—, —S—, —S(═O)—, —S(═O)₂—, —NR²¹—, —C(═O)—, —C(═O)NR²¹—, —NR²¹C(═O)—, —S(═O)₂NR²¹—, or —NR²¹S(═O)₂;
T² is absent or C₁-C₁₀alkylene optionally substituted with one or more R, and wherein one, two, three, or four carbon atoms of the C₁-C₁₀alkylene are optionally replaced with —S—, —S(═O)—, —S(═O)₂—, —O—, or —NR²¹—;
R²¹ is hydrogen, C₁-C₆alkyl, C₁-C₆haloalkyl, C₁-C₆hydroxyalkyl, C₁-C₆aminoalkyl, or C₁-C₆heteroalkyl;
(i) B² is absent;
or
(ii) B² is —N(R²³)₂;
each R²³ is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
or
(iii) B² is —CF₃;
or
(iv) B² is cycloalkyl, heterocycloalkyl, aryl, or heteroaryl; wherein the cycloalkyl, heterocycloalkyl, aryl, and heteroaryl is optionally substitued with one or more R²⁴;
each R²⁴ is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
or
(v) B² is 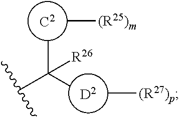
Ring C² is cycloalkyl, heterocycloalkyl, aryl, or heteroaryl;
each R²⁵ is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
m is 0, 1, 2, 3, or 4;
R²⁶ is hydrogen, C₁-C₆alkyl, C₁-C₆haloalkyl, C₁-C₆hydroxyalkyl, C₁-C₆aminoalkyl, or C₁-C₆heteroalkyl;
Ring D² is cycloalkyl, heterocycloalkyl, aryl, or heteroaryl;
each R²⁷ is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
p is 0, 1, 2, 3, or 4;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, -L-cycloalkyl, -L-heterocycloalkyl, -L-aryl, or -L-heteroaryl, wherein each alkyl, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, -L-cycloalkyl, -L-heterocycloalkyl, -L-aryl, or -L-heteroaryl, wherein each alkyl, alkenyl, alkynyl, cycloalkyl, heterocycloalkyl, aryl, and heteroaryl is independently optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, -L-cycloalkyl, -L-heterocycloalkyl, -L-aryl, or -L-heteroaryl, wherein each alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
L is absent or C₁-C₃alkylene optionally substituted with one or more R; and
each R is independently halogen, —CN, —OH, —SF₅, —SH, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, C₃-C₆cycloalkyl, or 3- to 6-membered heterocycloalkyl;
or two R on the same atom form an oxo;
or two R on the same or different atoms are taken together to form a cycloalkyl, heterocycloalkyl, aryl, or heteroaryl.

54. The conjugate of claim 53, wherein T¹ is absent or C₁-C₃alkylene.

55. The conjugate of claim 53 or 54, wherein B¹ is cycloalkyl optionally substitued with one or more R.

56. The conjugate of any one of claims 53-55, wherein B¹ is bicyclo[1.1.1]pentanyl, bicyclo[2.2.1]heptanyl, bicyclo[2.2.2]octanyl, or bicyclo[2.1.1]hexanyl.

57. The conjugate of any one of claims 53-55, wherein B¹ is bicyclo[1.1.1]pentanyl.

58. The conjugate of any one of claims 53-57, wherein S¹ is absent.

59. The conjugate of claim 53, wherein

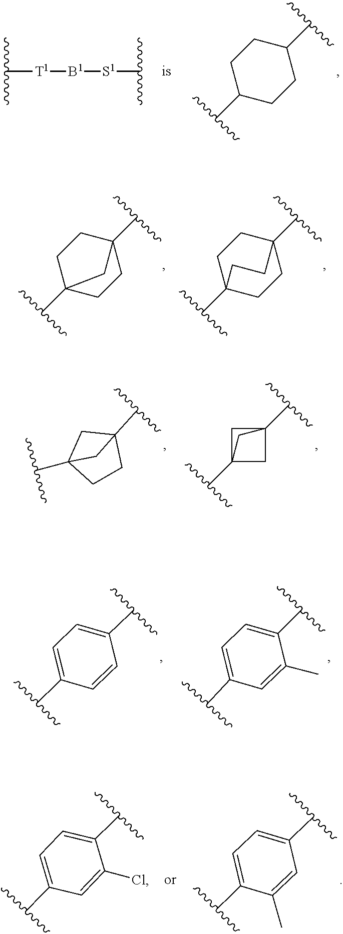

60. The conjugate of claim 53, wherein

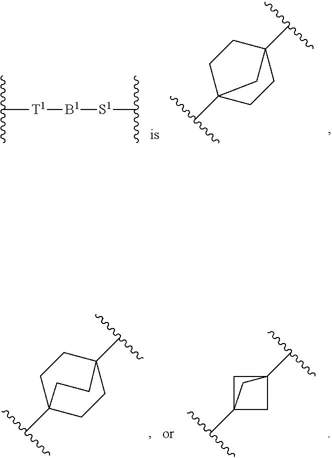

61. The conjugate of claim 53, wherein

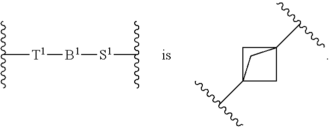

62. The conjugate of any one of claims 53-61, wherein A² is C₁-C₆alkylene optionally substitued with one or more R²².

63. The conjugate of any one of claims 53-61, wherein A² is heterocycloalkyl optionally substitued with one or more R²².

64. The conjugate of any one of claims 53-61, wherein A² is pyrrolidinyl, piperidinyl, piperazinyl, or morpholinyl, each optionally substitued with one or more R²².

65. The conjugate of any one of claims 53-61, wherein A² is piperidinyl optionally substitued with one or more R²².

66. The conjugate of any one of claims 53-61, wherein A² is 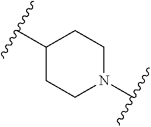 [SMILES: *C1CCN(*)CC1]
optionally substitued with one or more R²².

67. The conjugate of any one of claims 53-66, wherein each R²² is independently —C(═O)OR^{b} or —C(═O)NR^{c}R^{d}.

68. The conjugate of any one of claims 53-67, wherein each R²² is independently —C(═O)NR^{c}R^{d}.

69. A conjugate of Formula (C), or a pharmaceutically acceptable salt or stereoisomer thereof: 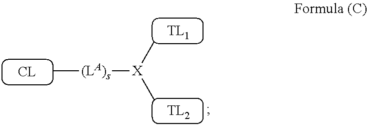
wherein:
L^{A} represents a linker;
s is 0 or 1;
CL represents a metal chelator;
X is N, optionally substituted CH, optionally substituted cycloalkyl, optionally substituted heterocycloalkyl, optionally substituted aryl, or optionally substituted heteroaryl;
TL₁ is of Formula (IC): 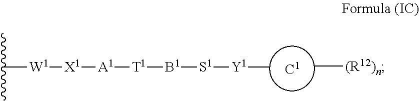
wherein:
W¹ is absent, —O—, —S—, —S(═O)—, —S(═O)₂—, —NR¹¹—, —C(═O)—, —C(═O)NR¹¹—, —NR¹¹C(═O)—, —S(═O)₂NR¹¹—, or —NR¹¹S(═O)₂;
X¹ is C₁-C₁₀alkylene optionally substituted with one or more R and wherein one, two, three, or four carbon atoms of the C₁-C₁₀alkylene are optionally replaced with —S—, —S(═O)—, —S(═O)₂—, —O—, or —NR¹¹—;
A¹ is absent, cycloalkyl, heterocycloalkyl, aryl, or heteroaryl, wherein the cycloalkyl, heterocycloalkyl, aryl, and heteroaryl is optionally substitued with one or more R;
T¹ is absent or C₁-C₃alkylene optionally substituted with one or more R and wherein one carbon atom of the C₁-C₃alkylene is optionally replaced with —C(═O)—, —S—, —S(═O)—, —S(═O)₂—, —O—, —NR¹¹—;
B¹ is absent, C₁-C₁₀alkylene, cycloalkyl, heterocycloalkyl, aryl, or heteroaryl, wherein the alkylene, cycloalkyl, heterocycloalkyl, aryl, and heteroaryl is optionally substitued with one or more R, and wherein one, two, three, or four carbon atoms in the C₁-C₁₀alkylene are optionally replaced with —S—, —S(═O)—, —S(═O)₂—, —O—, —NR¹¹—;
S¹ is absent or C₁-C₃alkylene optionally substituted with one or more R and wherein one or two carbon atoms of the C₁-C₃alkylene are optionally replaced with —S—, —S(═O)—, —S(═O)₂—, —O—, or —NR¹¹—;
Y¹ is absent, —O—, —S—, —S(═O)—, —S(═O)₂—, —NR¹¹—, —C(═O)—, —C(═O)NR¹¹—, —NR¹¹C(═O)—, —S(═O)₂NR¹¹—, or —NR¹¹S(═O)₂;
R¹¹ is hydrogen, C₁-C₆alkyl, C₁-C₆haloalkyl, C₁-C₆hydroxyalkyl, C₁-C₆aminoalkyl, or C₁-C₆heteroalkyl;
Ring C¹ is cycloalkyl, heterocycloalkyl, aryl, or heteroaryl;
each R¹² is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
n is 0, 1, 2, 3, 4, 5, or 6;
TL₂ is of Formula (IIC): 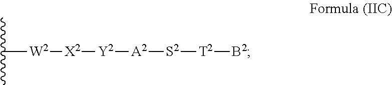
wherein:
W² is absent, —O—, —S—, —S(═O)—, —S(═O)₂—, —NR²¹—, —C(═O)—, —C(═O)NR²¹—, —NR²¹C(═O)—, —S(═O)₂NR²¹—, or —NR²¹S(═O)₂;
X² is absent or C₁-C₁₀alkylene optionally substituted with one or more R, and wherein one, two, three, or four carbon atoms of the C₁-C₁₀alkylene are optionally replaced with —S—, —S(═O)—, —S(═O)₂—, —O—, or —NR²¹—;
Y² is absent, —O—, —S—, —S(═O)—, —S(═O)₂—, —NR²¹—, —C(═O)—, —C(═O)NR²¹—, —NR²¹C(═O)—, —S(═O)₂NR²¹—, or —NR²¹S(═O)₂;
A² is cycloalkyl, heterocycloalkyl, aryl, or heteroaryl, wherein the cycloalkyl, heterocycloalkyl, aryl, and heteroaryl is optionally substitued with one or more R²²;
each R²² is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
S² is absent, —O—, —S—, —S(═O)—, —S(═O)₂—, —NR²¹—, —C(═O)—, —C(═O)NR²¹—, —NR²¹C(═O)—, —S(═O)₂NR²¹—, or —NR²¹S(═O)₂;
T² is absent or C₁-C₁₀alkylene optionally substituted with one or more R, and wherein one, two, three, or four carbon atoms of the C₁-C₁₀alkylene are optionally replaced with —S—, —S(═O)—, —S(═O)₂—, —O—, or —NR²¹—;
R²¹ is hydrogen, C₁-C₆alkyl, C₁-C₆haloalkyl, C₁-C₆hydroxyalkyl, C₁-C₆aminoalkyl, or C₁-C₆heteroalkyl;
(i) B² is absent;
or
(ii) B² is —N(R²³)₂;
each R²³ is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
wherein each alkyl, alkenyl, alkynyl, cycloalkyl, heterocycloalkyl, aryl, and heteroaryl is optionally and independently substituted with one or more R;
or
(iii) B² is —CF₃;
or
(iv) B² is cycloalkyl, heterocycloalkyl, aryl, or heteroaryl; wherein the cycloalkyl, heterocycloalkyl, aryl, and heteroaryl is optionally substitued with one or more R²⁴;
each R²⁴ is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
or
(v) B² is 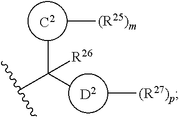
Ring C² is cycloalkyl, heterocycloalkyl, aryl, or heteroaryl;
each R²⁵ is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
m is 0, 1, 2, 3, or 4;
R²⁶ is hydrogen, C₁-C₆alkyl, C₁-C₆haloalkyl, C₁-C₆hydroxyalkyl, C₁-C₆aminoalkyl, or C₁-C₆heteroalkyl;
Ring D² is cycloalkyl, heterocycloalkyl, aryl, or heteroaryl;
each R²⁷ is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
p is 0, 1, 2, 3, or 4;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, -L-cycloalkyl, -L-heterocycloalkyl, -L-aryl, or -L-heteroaryl, wherein each alkyl, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, -L-cycloalkyl, -L-heterocycloalkyl, -L-aryl, or -L-heteroaryl, wherein each alkyl, alkenyl, alkynyl, cycloalkyl, heterocycloalkyl, aryl, and heteroaryl is independently optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, -L-cycloalkyl, -L-heterocycloalkyl, -L-aryl, or -L-heteroaryl, wherein each alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
L is absent or C₁-C₃alkylene optionally substituted with one or more R; and
each R is independently halogen, —CN, —OH, —SF₅, —SH, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, C₃-C₆cycloalkyl, or 3- to 6-membered heterocycloalkyl;
or two R on the same atom form an oxo;
or two R on the same or different atoms are taken together to form a cycloalkyl, heterocycloalkyl, aryl, or heteroaryl.

70. The conjugate of claim 69, wherein T¹ is absent.

71. The conjugate of claim 69 or 70, wherein B¹ is C₁-C₆alkylene.

72. The conjugate of any one of claims 69-71, wherein B¹ is cycloalkyl optionally substitued with one or more R.

73. The conjugate of any one of claims 69-71, wherein B¹ is a monocyclic cycloalkyl optionally substitued with one or more R.

74. The conjugate of any one of claims 69-71, wherein B¹ is a bicyclic cycloalkyl optionally substitued with one or more R.

75. The conjugate of any one of claims 69-71, wherein B¹ is bicyclo[1.1.1]pentanyl, bicyclo[2.2.1]heptanyl, bicyclo[2.2.2]octanyl, or bicyclo[2.1.1]hexanyl.

76. The conjugate of any one of claims 69-71, wherein B¹ is bicyclo[1.1.1]pentanyl.

77. The conjugate of any one of claims 69-76, wherein S¹ is absent.

78. The conjugate of claim 69, wherein

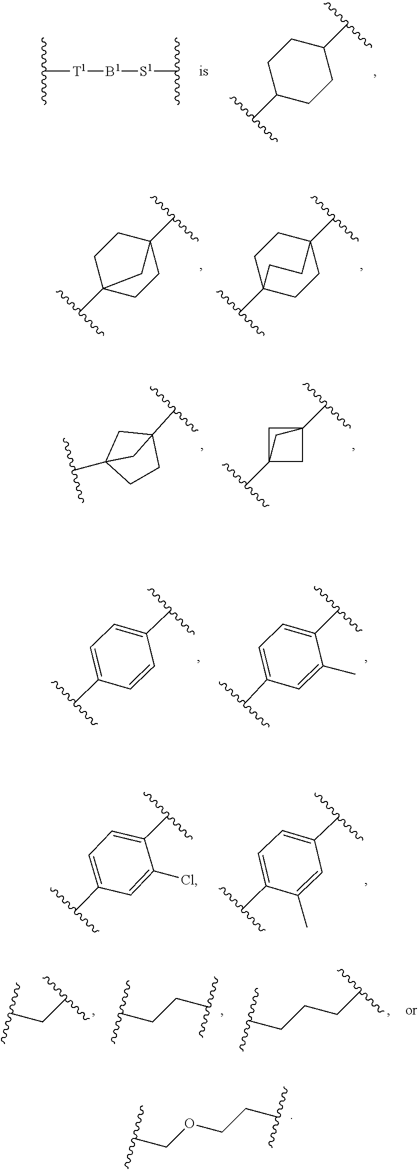

79. The conjugate of claim 69, wherein

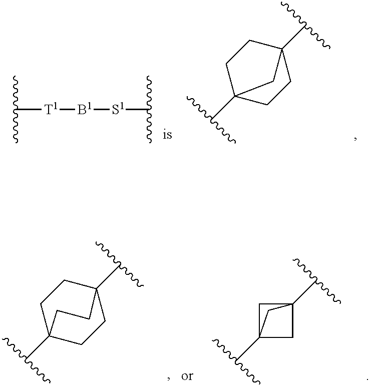

80. The conjugate of claim 69, wherein

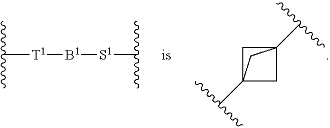

81. The conjugate of any one of claims 69-80, wherein A² is heterocycloalkyl optionally substitued with one or more R²².

82. The conjugate of any one of claims 69-80, wherein A² is pyrrolidinyl, piperidinyl, piperazinyl, or morpholinyl, each optionally substitued with one or more R²².

83. The conjugate of any one of claims 69-80, wherein A² is piperidinyl optionally substitued with one or more R²².

84. The conjugate of any one of claims 69-80, wherein A² is 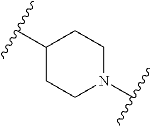 [SMILES: *C1CCN(*)CC1]
optionally substitued with one or more R²².

85. The conjugate of any one of claims 69-80, wherein A² is

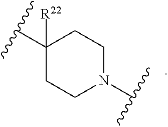

86. The conjugate of any one of claims 69-85, wherein each R²² is independently —C(═O)OR^{b} or —C(═O)NR^{c}R^{d}.

87. The conjugate of any one of claims 69-86, wherein each R²² is independently —C(═O)NR^{c}R^{d}.

88. The conjugate of any one of claims 53-87, wherein X is CH.

89. The conjugate of any one of claims 53-87, wherein X is optionally substituted heterocycloalkyl.

90. The conjugate of any one of claims 53-89, wherein W¹ is absent, —C(═O)—, or —C(═O)NR¹¹—.

91. The conjugate of any one of claims 53-90, wherein X¹ is C₁-C₄alkylene.

92. The conjugate of any one of claims 53-91, wherein

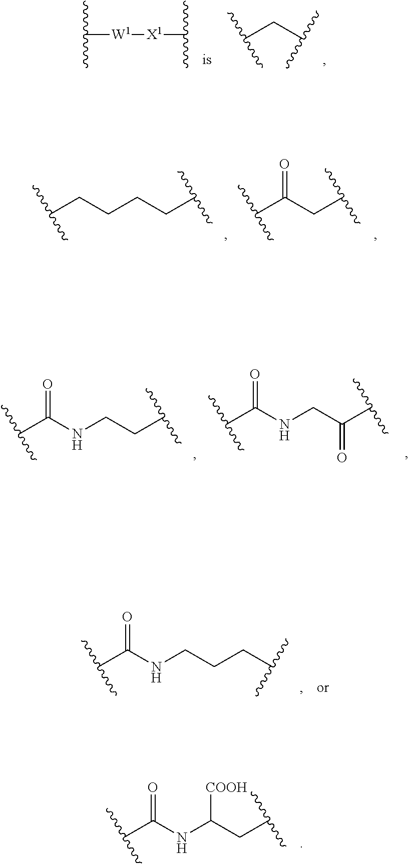

93. The conjugate of any one of claims 53-92, wherein

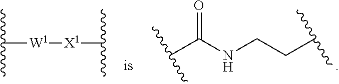

94. The conjugate of any one of claims 53-93, wherein A¹ is heteroaryl optionally substitued with one or more R.

95. The conjugate of any one of claims 53-93, wherein A¹ is triazolyl optionally substitued with one or more R.

96. The conjugate of any one of claims 53-92, wherein A¹ is 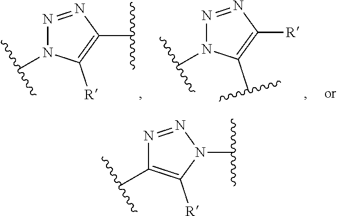
wherein R′ is hydrogen or R.

97. The conjugate of any one of claims 53-96, wherein T¹ is absent or C₁-C₃alkylene.

98. The conjugate of any one of claims 53-97, wherein Y¹ is absent, —O—, —S—, —S(═O)₂—, —C(═O)NR¹¹—, or —NR¹¹C(═O)—.

99. The conjugate of any one of claims 53-98, wherein Ring C¹ is heteroaryl or phenyl.

100. The conjugate of any one of claims 53-98, wherein Ring C¹ is monocyclic heteroaryl.

101. The conjugate of any one of claims 53-98, wherein Ring C¹ is bicyclic heteroaryl.

102. The conjugate of any one of claims 53-98, wherein 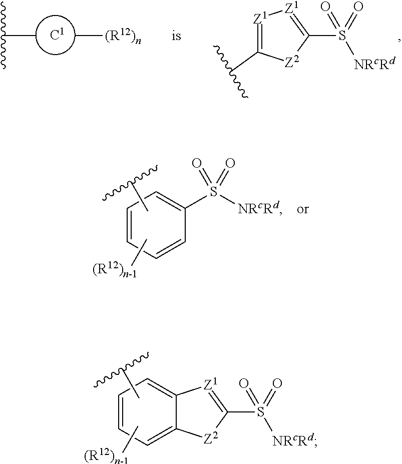
wherein each Z¹ is independently —N— or —CR¹²′—;
Z² is —O—, —S—, or —NR¹²″—;
each R¹²′ is independently hydrogen, halogen, —CN, —OH, —OR^{a}, —NR^{c}R^{d}, C₁-C₆alkyl, C₁-C₆haloalkyl, cycloalkyl, or heterocycloalkyl; and
R¹²″ is hydrogen, —S(═O)R^{a}, —S(═O)₂R^{a}, —S(═O)₂NR^{c}R^{d}, —C(═O)R^{a}, —C(═O)OR^{b}, —C(═O)NR^{c}R^{d}, C₁-C₆alkyl, C₁-C₆haloalkyl, cycloalkyl, or heterocycloalkyl.

103. The conjugate of any one of claims 53-98, wherein

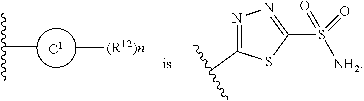

104. The conjugate of any one of claims 53-98, wherein

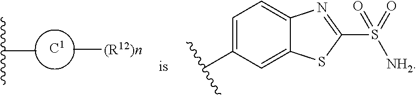

105. The conjugate of any one of claims 53-98, wherein

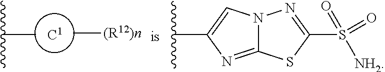

106. The conjugate of any one of claims 53-105, wherein W² is absent, —C(═O)—, or —NR²¹C(═O)—.

107. The conjugate of any one of claims 53-106, wherein X² is C₁-C₄alkylene optionally substituted with one or more R.

108. The conjugate of any one of claims 53-107, wherein X² is —CH₂—.

109. The conjugate of any one of claims 53-108, wherein Y² is absent, —NR²¹—, or —NR²¹C(═O)—.

110. The conjugate of any one of claims 53-109, wherein Y² is —NR²¹C(═O)—.

111. The conjugate of any one of claims 53-110, wherein

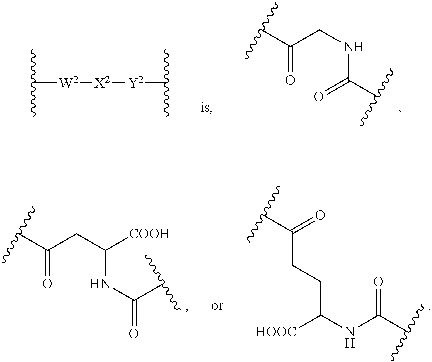

112. The conjugate of any one of claims 53-111, wherein S² is absent, —NR²¹—, —C(═O)—, or —NR²¹C(═O)—.

113. The conjugate of any one of claims 53-112, wherein T² is absent or C₁-C₆alkylene optionally substituted with one or more R.

114. The conjugate of any one of claims 53-113, wherein

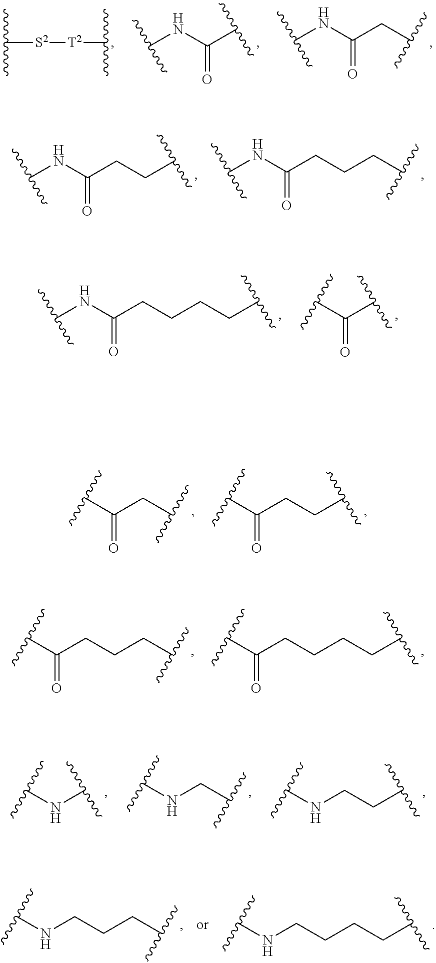

115. The conjugate of any one of claims 53-114, wherein

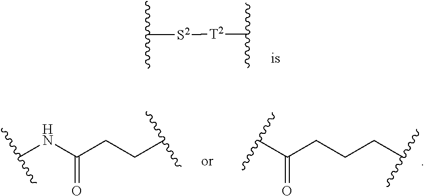

116. The conjugate of any one of claims 53-115, wherein B² is cycloalkyl, heterocycloalkyl, aryl, or heteroaryl; wherein the cycloalkyl, heterocycloalkyl, aryl, and heteroaryl is optionally substitued with one or more R²⁴.

117. The conjugate of any one of claims 53-116, wherein each R²⁴ is independently halogen, —OH, C₁-C₆alkyl, or C₁-C₆haloalkyl.

118. The conjugate of any one of claims 53-115, wherein B² is 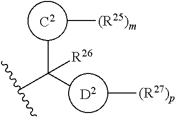
wherein:
Ring C² is cycloalkyl, heterocycloalkyl, aryl, or heteroaryl;
each R²⁵ is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R;
m is 0, 1, 2, 3, or 4;
R²⁶ is hydrogen, C₁-C₆alkyl, C₁-C₆haloalkyl, C₁-C₆hydroxyalkyl, C₁-C₆aminoalkyl, or C₁-C₆heteroalkyl;
Ring D² is cycloalkyl, heterocycloalkyl, aryl, or heteroaryl;
each R²⁷ is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally and independently substituted with one or more R; and
p is 0, 1, 2, 3, or 4.

119. The conjugate of any one of claims 53-115, wherein B² is

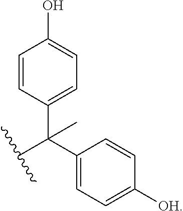 [SMILES: *C(C)(c1ccc(O)cc1)c1ccc(O)cc1]

120. The conjugate of any one of claims 1-119, wherein s is 0.

121. The conjugate of any one of claims 1-119, wherein s is 1.

122. The conjugate of any one of claims 1-119 or 121, wherein the linker L^{A} has a structure of Formula (L-1) 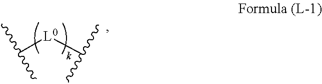
wherein:
each L⁰ is independently —O—, —NR^{L}—, —N(R^{L})₂⁺—, —OP(═O)(OR^{L})O—, —S—, —S(═O)—, —S(═O)₂—, —CH—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S)NR^{L}—, —CR^{L}═N—, —N═CR^{L}, —NR^{L}S(═O)₂—, —S(═O)₂NR^{L}—, —C(═O)NR^{L}S(═O)₂—, —S(═O)₂NR^{L}C(═O)—, cycloalkylene, heterocycloalkylene, arylene, heteroarylene, C₁-C₃₀alkylene, C₂-C₃₀alkenylene, C₂-C₃₀alkynylene, C₁-C₃₀heteroalkylene, —(C₁-C₃₀alkylene)-O—, —O—(C₁-C₃₀alkylene)-, —(C₁-C₃₀alkylene)-NR^{L}—, —NR^{L}—(C₁-C₃₀alkylene)-, —(C₁-C₃₀alkylene)-N(R^{L})₂⁺—, or —N(R^{L})₂⁺—(C₁-C₃₀alkylene)-; wherein each cycloalkylene, heterocycloalkylene, arylene, heteroarylene, alkylene, alkenylene, alkynylene, and heteroalkylene is optionally independently substituted with one or more R;
each R^{L} is independently hydrogen, C₁-C₄alkyl, C₁-C₄haloalkyl, C₁-C₄heteroalkyl, C₂-C₆alkenyl, C₂-C₆alkynyl, cycloalkyl, heterocycloalkyl, aryl, or heteroaryl;
k is 1-20; and
each R is independently halogen, —CN, —OH, —SF₅, —SH, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, C₃-C₆cycloalkyl, or 3- to 6-membered heterocycloalkyl;
or two R on the same atom form an oxo.

123. The conjugate of claim 122, wherein the linker L^{A} has a structure of Formula (L-1a), 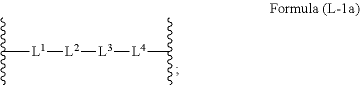
wherein:
L¹ is absent, —O—, —NR^{L}—, —N(R^{L})₂⁺—, —S—, —S(═O)—, —S(═O)₂—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}S(═O)₂—, —S(═O)₂NR^{L}—, —C(═O)NR^{L}S(═O)₂—, or —S(═O)₂NR^{L}C(═O)—;
L² is 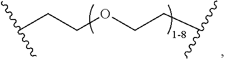
C₁-C₁₀alkylene, or heterocycloalkylene optionally substituted with one or more R;
L³ is absent or C₁-C₁₀alkylene; and
L⁴ is absent, —O—, —NR^{L}—, —N(R^{L})₂⁺—, —S—, —S(═O)—, —S(═O)₂—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}S(═O)₂—, —S(═O)₂NR^{L}—, —C(═O)NR^{L}S(═O)₂—, or —S(═O)₂NR^{L}C(═O)—.

124. The conjugate of any one of claims 1-119 or 121-123, wherein the linker L^{A} is

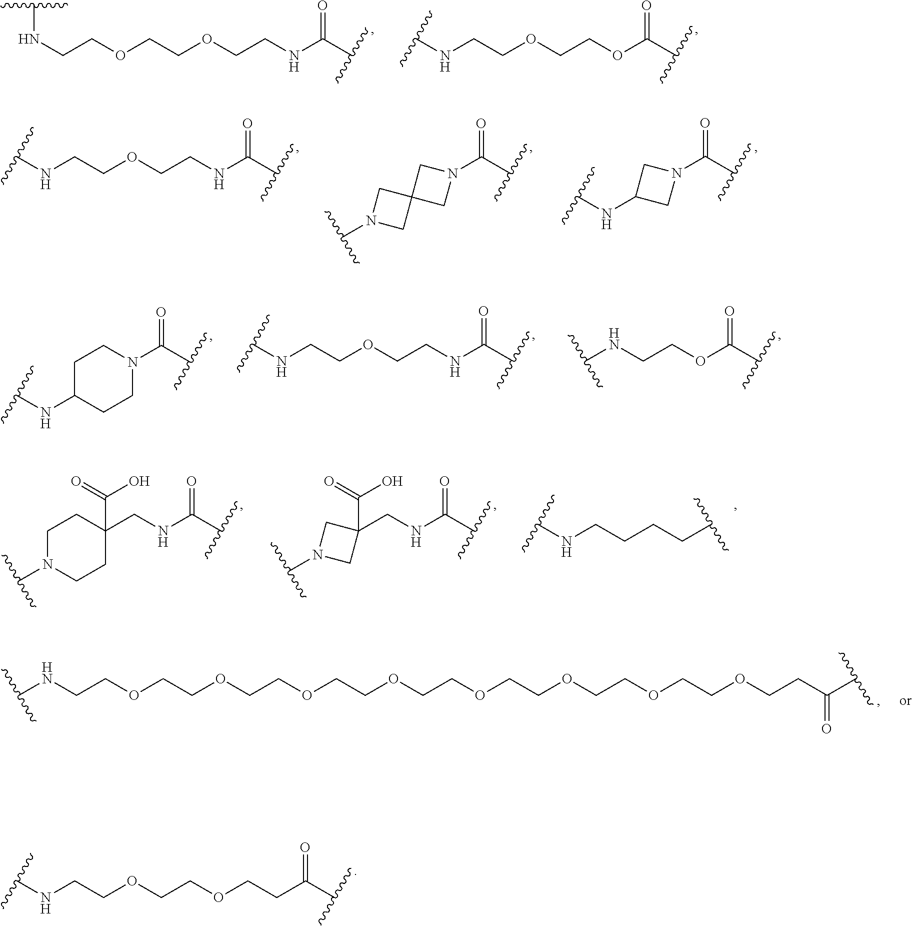

125. The conjugate of claim 124, wherein the metal chelator is

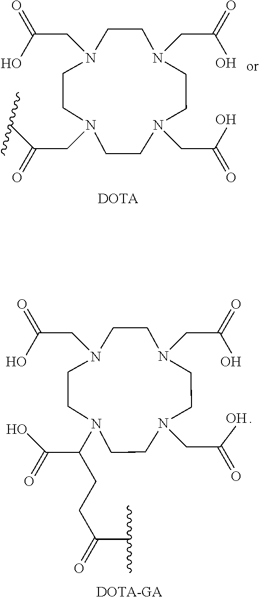

126. The conjugate of any one of claims 1-125, wherein the conjugate is found in table 1.

127. The conjugate of any one of claims 1-126, further comprising a radionuclide bound to the metal chelator to form a radiopharmaceutical conjugate.

128. The radiopharmaceutical conjugate of claim 127, wherein the radionuclide is an alpha particle-emitting radionuclide.

129. The radiopharmaceutical conjugate of claim 128, wherein the alpha particle-emitting radionuclide is Ac-225.

130. The radiopharmaceutical conjugate of claim 127, wherein the radionuclide is a beta particle-emitting radionuclide.

131. The radiopharmaceutical conjugate of claim 130, wherein the beta particle-emitting radionuclide is lutetium-177.

132. The radiopharmaceutical conjugate of claim 127, wherein the radionuclide is a positron-emitting radionuclide.

133. A conjugate having a structure of Formula (AC), (AC′), (BC), (BC′), (CC), or (CC′), wherein each of the formula is described herein.

134. A pharmaceutical composition comprising a radiopharmaceutical conjugate of any one of claims 128-133, and a pharmaceutically acceptable excipient or carrier.

135. A method of diagnosing or imaging a disease or disorder in a subject in need thereof, the method comprising administering to the subject the radiopharmaceutical conjugate of any one of claims 128-133, or the pharmaceutical composition of claim 134.

136. A method of treating a disease or disorder in a subject in need thereof, the method comprising administering to the subject the radiopharmaceutical conjugate of any one of claims 128-133, or the pharmaceutical composition of claim 134.

137. The method of claim 135 or 136, wherein the disease or disorder is a cancer.

138. The method of claim 137, wherein the cancer is hypoxic.

139. The method of any one of claim 137 or 138, wherein the cancer is cell renal cell carcinoma (ccRCC).
